(19) United States
(12) Patent Application Publication
(10) Pub. No.: US 2003/0157113 A1
(43) Pub. Date: Aug. 21, 2003

(54) Compositions and methods for treatment of neoplastic disease

(76) Inventor: David S. Terman, Pebble Beach, CA (US)

Correspondence Address: David S. Terman, P.O. Box 987, Pebble beach, CA, 93953, US

(21) Appl. No.: 09/751,708

(22) Filed: Dec. 28, 2000

Related U.S. Application Data
(60) Provisional application No. 60/173,371, filed on Dec. 28, 1999.

(51) Int. Cl. A61K 39/00; C12N 5/12

(52) U.S. Cl. 424/184.1; 435/346

(57) ABSTRACT

The present invention comprises compositions and methods for treating a tumor or neoplastic disease in a host, The methods employ conjugates comprising superantigen polypeptides, nucleic acids with other structures that preferentially bind to tumor cells and are capable of inducing apoptosis. Also provided are superantigen-glycolipid conjugates and vesicles that are loaded onto antigen presenting cells to activate both T cells and NKT cells. Cell-based vaccines comprise tumor cells engineered to express a superantigen along with glycolipids products which, when expressed, render the cells capable of eliciting an effective anti-tumor immune response in a mammal into which these cells are introduced. Included among these compositions are tumor cells, hybrid cells of tumor cells and accessory cells, preferably dendritic cells. Also provided are tumoricidal T cells and NKT cells devoid of inhibitory receptors or inhibitory signaling motifs which are hyperresponsive to the the above compositions and lipid-based tumor associated antigens that can be administered for adoptive immunotherapy of cancer and infectious diseases.

### BACKGROUND OF THE INVENTION

1. Field of the Invention

The invention relates generally to immunotherapeutic compositions and methods for treating tumors and cancer. The methods are based on the expression of superantigen (“SAg”) alone or in combination with other molecules in transfected host cells (tumor cells, accessory cells or lymphocytes). Other therapeutic methods are based on administering T cells which are activated by cells engineered to express SAg and other immunostimulatory molecules and structures.

2. Description of the Background Art

Therapy of the neoplastic diseases has largely involved the use of chemotherapeutic agents, radiation, and surgery. However, results with these measures, while beneficial in some tumors, has had only marginal effects in many patients and little or no effect in many others, while demonstrating unacceptable toxicity. Hence, there has been a quest for newer modalities to treat neoplastic diseases.

In 1980, tumoricidal effects were demonstrated in four of five patients with advanced breast cancer treated with autologous plasma that had been perfused over columns in which Staphylococcal Protein A was chemically attached to a solid surface (Terman et al., New Eng. J. Med., 305:1195 (1981)). While the initial observations of tumor killing effects with the immobilized Protein A perfusion system have been confirmed, some have obtained inconsistent results.

The explanation of these inconsistencies appears to be as follows. First, commercial Protein A is an impure preparation, as evident from polyacrylamide gel electrophoresis and radioinmiunoassays that detected Staphylococcal enterotoxins in the preparations. Second, various methods of immobilizing Protein A to solid supports have been used, sometimes resulting in loss of biological activity of the perfusion system. Third, the plasma used for perfusion over immobilized Protein A has often been stored and treated in different ways, also resulting in occasional inactivation of the system. Moreover, the substance(s) or factors responsible for the anti-tumor effect of this extremely complex perfusion system have not been previously defined. The system contained an enormous number of biologically active materials, including the Protein A itself, Staphylococcal proteases, nucleases, exotoxins, enterotoxins and leukocidin, as well as the solid support and coating materials. In addition, several anaphylatoxins were generated in plasma after contact with immobilized Protein A. Finally, it was speculated that the biological activity of the system was due to the removal from the plasma by the Protein A of immunosuppressive immune complexes that otherwise inhibit the patient's antitumor immune response.

The Staphylococcal enterotoxins that contaminate the Protein A columns are a family of extracellular products of Staphylococcal aureus that belong to a well recognized group of proteins that have common physical and chemical properties. The enterotoxins produce a number of characteristic effects in humans and animals, such as emesis, hypotension, fever, chills, and shock in primates and enhancement of gram negative endotoxic lethality in rabbits. At least some of these effects are due to the ability of these proteins to act as extremely potent T cell mitogens.

Staphylococcal enterotoxins are representative of a family of molecules known as SAgs which are the most powerful T cell mitogens known. They are capable of activating 5 to 30% or the total T cell population compared to 0.01% for conventional antigens. Moreover, the enterotoxins elicit strong polyclonal proliferation at concentrations 10³—fold lower than conventional T cell mitogens. The most potent enterotoxin, Staphylococcal enterotoxin A (SEA), has been shown to stimulate DNA synthesis in human T cells at concentrations of as low as 10⁻¹³ to 10⁻¹⁶M. Enterotoxin-activated T cells produce a variety of cytokines, including IFN, various interleukins and TNF. Enterotoxins stimulate several other cell populations involved in innate and adaptive immunity which also play a major role in anti-tumor immunity, For example, enterotoxins engage the variable region of the TCR chain on exposed face of the pleated sheet and the sides of the MHC class II molecule.

The SAg is capable of augmenting the TH-1 cytokine response by CD4+ cells while also activating NKT and NK cells. NK cell cytotoxicity is augmented by IFNγ produced by SAg activated T cells. NKT cells are known to be activated by SAgs, peptides, -galactosylceramides and lipoarabinomannans presented on CD1 receptors. Evidence points to an invariant lectin like recognition unit on the NKT cell chain as a specific ligand for galactosylceramide determinants on tumor cells. SAgs induce tumor killing in vivo when given alone or conjugated to tumor associated antibodies. They are also effective when employed ex vivo to produce tumor sensitized T cells for the adoptive therapy of MCA 205/207 tumors. SAg transfected tumor cells have shown a capacity to reduce metastatic disease in a murine mammary carcinoma model.

In addition to these common biological activities, the Staphylococcal enterotoxins share common physicochemical properties. They are heat stable, trypsin resistant, and soluble in water and salt solutions. Furthermore, the Staphylococcal enterotoxins have similar sedimentation coefficients, diffusion constants, partial specific volumes, isoelectric points, and extinction coefficients. The Staphylococcal enterotoxins have been divided into five serological types designated SEA, Staphylococcal enterotoxin B (SEB), Staphylococcal enterotoxin C (SEC), Staphylococcal enterotoxin D (SED), and Staphylococcal enterotoxin E (SEE), which exhibit striking structural similarities. The enterotoxins are composed of a single polypeptide chain of about 30 kilodaltons (kD). All staphylococcal enterotoxins have a characteristic disulfide loop near the middle of the molecule. SEA is a flat monomer consisting or 233 amino acid residues divided into two domains. Domain I comprises residues 31-116 and domain II of residues 117-233 together with the amino tail 1-30. In addition, the biologically active regions of the proteins are conserved and show a high degree of homology. One region of striking amino acid sequence homology between SEA, SEB, SEC, SED, and SEE is located immediately downstream (toward the carboxy terminus) from the cysteine located at residue 106 in SEA. This region is thought to be responsible for T cell activation. A second homologous region that begins at residue 147 and extends downstream is highly conserved. This region is believed to mediate emetic activity. The region related to emetic activity can be omitted from enterotoxins used as therapeutics.

A sequence analysis of the Staphylococcal enterotoxins with other toxins has revealed SEA, SEB, SEC, SED, Staphylococcal toxic shock-associated toxin (TSST-1 also known as SEF), and the Streptococcal exotoxins share considerable nucleic acid and amino acid sequence homology. The enterotoxins belong to a common generic group of proteins thought to be evolutionarily related.

Enterotoxins bind to MHC Class II molecules and the T cell receptor (“TCR”) in a manner quite distinct from conventional antigens. Enterotoxins engage the variable region of the TCR β chain on an exposed face of the β pleated sheet and the sides of the MHC Class II molecule, rather than engaging the groove of the Class II molecule like conventional antigens. In contrast to SEB and the SEC, which have only the capacity to bind to the MHC class II α chain, SEA, as well as SEE and SED, also interacts with the MHC class II β chain.in a zinc dependent manner T cell recognition is based on the presence of the β chain and is therefore independent of other TCR components and diversity elements. Single amino acid positions and regions important for SAg-TCR interactions have been defined. These residues are located in the vicinity of the shallow cavity formed between the two domains. The alanine substitution of amino acid residue Asn23 in SEB has demonstrated the importance of this residue in SEB/TCR interaction. This particular residue is conserved among all of the Staphylococcal enterotoxins and may constitute a common anchor position for enterotoxin interaction with TCR Vβ chains. Amino acid residues in positions 60-64 have also been shown to contribute to the TCR interaction as do the cysteine residues forming the intermolecular disulfide bridge of SEA. For SEC2 and SEC3, the key points of interaction in the Vβ chain are located in the CDR1, CDR2 and HRV4 TCR Vβ-3 chain. Hence, multiple and highly variable parts of the Vβ chain contribute to the formation of the enterotoxin binding site on the TCR. Thus far, a single and linear consensus motif in the TCR Vβ displaying a high affinity interaction with particular enterotoxins has not been identified. A significant contribution of the TCR α chain in enterotoxin-TCR recognition is acknowledged as well as MHC class II isotypes. This distinctive binding mechanism of enterotoxins which bypasses the highly variable parts of the MHC class II and TCR molecules allows them to activate a high frequency or T cells with massive lymphoproliferation, cytokine induction and cytotoxic T cell generation. These properties are shared by other proteins made by infectious agents. Together, these proteins form a well recognized group known as SAgs.

There are two general classes of SAgs. The first includes minor lymphocyte stimulating (MLS) antigens. The second class of SAgs includes mycoplasmal, viral, and bacterial proteins such as the Staphylococcal enterotoxins.

Streptococcal exotoxins. All SAgs have the following properties. T cell activation does not require antigen processing. There is no MHC restriction of responding T cells. SAgs bind to and evoke responses from all T cells expressing Vβ receptors, without requiring other TCR or diversity elements. CD4-CD8-α/β T cells and γ/δ T cells arc also capable of responding to SAgs. The SAgs induce a biochemically distinct T cell activation pathway. Thus, SAgs interact with and activate a much larger proportion of T cells than conventional antigens, causing massive lymphoproliferation, cytotoxic T cell generation, and cytokine secretion. A given SAg can activate up to 30% of resting T cells compared to 0.01% for conventional antigens. As highly representative members of this family of SAgs, the enterotoxins share these characteristics.

The present invention features the use of SAgs in association with molecules to produce tumor killing effects. The SAgs are useful in peptide form and may combine with another peptide or nucleic acid to form a conjugate. The effect of the combined molecules is synergistic. These conjugates are useful when administered as a preventative or therapeutic antitumor vaccine in tumor bearing patients. Alternatively, they may be used ex vivo to load an antigen presenting cell as a means of immunizing a T, NK or NKT cell population for use in adoptive therapy of cancer. Examples of such conjugates are complexes between: SAg and glycosylceramide; SAg and apolipoproteins (Lp(a)), SAg and oxyLDL, SAg and verotoxins, SAg and GPI-ceramide (with phytosphingosine backbone), SAg and lipopolysaccharide (LPS), SAg and peptidoglycan, SAg and mannan proteoglycan, SAg and muramic acid, SAg and phytosphingolipid, SAg and tumor peptides. Also intended are SAg and Gal conjugates and glycosylated SAgs.

The present invention features the use of SAg in association or conjugated to oxidized low density lipoproteins (oxyLDL) and apolipoproteins (e.g., lipoprotein (a) (Lp(a)). OxyLDL and its byproducts bind to receptors on sinusoidal endothelial cells in the tumor microcirculation where they induce apoptosis, increase levels of tissue factor and activated thrombin, upregulate achesion molecules and produce a prothrombotic state. Lp(a) is densely deposited in tumor microcirculation and as a competitive inhibitor of plasminogen is prothrombotic. Hence, both apolipoproteins and oxyLDL not only home to receptors on the tumor microcirculation but they also induce endothelial cell or macrophage apoptosis as well as a prothrombotic state. These local effects are amplified by the presence of the conjugated superantigen which induce a localized T cell immune and inflammatory response collectively resulting in a potent anti-tumor response.

The present invention also features the use of the SAg in association or conjugated to verotoxins. The latter molecules have the capacity to bind to galactosylceramide receptors on tumor cells and induce apoptosis. Hence, the tumor targeting and apoptosis inducing functions of the verotoxin are coupled with the T cell immune and inflammatory response induced by the SAgs to produce a potent and well localized anti-tumor response.

The present invention features the use of SAgs in association or conjugated to mono or digalactosylceramides. The latter have been isolated from human kidney, Fabry's disease kidney, marine sponge Aegelus mauritanius and is expressed in certain bacteria such as Sphingomonas paucimobilis. They have been shown to activate NKT cells and to induce anti-tumor effects in vivo against several types of tumors. The activation of NKT cells in the presence of the mono and digalactosylceramides appears to be IL-12 dependent. The biological activity of the -galactosylceramides is observed in both mono and digalactosylceramide forms and is dependent upon the presence of an anomeric configuration on the terminal galactose. The lengths of the sphingosine base and fatty acyl chains of 23 and 15 respectively also appear to be optimal for production of the anti-tumor effects. SAg is also used in association with phyosphinosine which is expressed in Saccharomyces cervevisiae membranes and vesicles.

SAgs are known to be the most powerful T cell mitogens known and have been shown to produce anti-tumor effects in several animal models. The -galactosylceramides are known to be potent inducers of NKT cell activation which have been shown to produce an anti-tumor effect in an IL-12 dependent manner. In the present invention SAgs are combined with -galactosylceramides biochemically as conjugates and genetically within a cell which expresses the newly synthesized protein-bound galactosylceramide on the a cell surface. The newly synthesized conjugates in native form or expressed in or on the cell produce a synergistic anti-tumor effect due to the activation of T cells and NKT cell populations.

Furthermore, in the present invention the SAg-galactosylceramides are expressed in tumor cells, dendritic cells (“DC”) or a hybrid cell made by fusing a tumor cell and a DC. The use of DCs or DC/tumor cell hybrids (DC/tc) to present the SAg-galactosylceramides fusion constructs or conjugates provides the optimal costimulation for activation of a tumor specific T cell population. The use of a tumor cell or a DC/tc provides in addition to costimulation, expression of the tumor antigen itself to activate anti-tumor T and NKT cell clones which are tumor specific. Hence, an optimal cell is a DC/tc which expresses SAg and SAg-anomeric galactosylceramides.

The SAg-galactosylceramide conjugates are useful in the present invention. However, there are distinct differences and advantages to producing and expressing the SAg-galactosylceramide conjugates within a cell. First, final products are quite different. One involves the enterotoxin-α-galactosylceramide in free form whereas the other involves cell associated enterotoxin-α-galactosylceramide which includes enterotoxin nucleic acids and peptides. In the cell both enterotoxins and —α-galactosylceramides are associated with numerous intracellular and membrane structures such as MHC, costimulatory and adhesion molecules, heat shock proteins, membrane glycolipids and glycosphingolipids which may improve immunogenicity and antigen presentation. They may also be transported in various vesicles and exosomes which may provide additional immunogenicity. With the addition of appropriate signals sequences and association with molecules involved in the antigen presenting pathways such as the invariant chain, TAP and LAMP molecules, the conjugates may be routed in the cell to the MHC class I, class II or CD1 receptor. Therefore, enterotoxin and -galactosylceramides produced within a cell is presented to the host's immune system in an entirely different form compared to the purified enterotoxin polypeptide.

Unlike free enterotoxin polypeptide or -galactosylceramide, SAg transfected tumor cells, DCs or DC/tc present enterotoxins to the T cell system in association and or conjugated to tumor associated antigens including mutated normal structures or fusion structures, costimulatory and adhesion molecules.. Indeed, the coadministration of SAg with tumor antigen would be expected to produce a heightened response to the tumor antigens while preventing the clonal deletion which occurs with SAg alone. Liu et al., Proc. Natl. Acad. Sci., 88: 8705-8709, (1991); McCormack et al., Proc. Natl. Acad. Sci., 91: 2086-2090, (1994); Coppola et al., Int. Immunol., 9: 1393-403, (1997). Hence, the coadministration of SAg-galactosylceramide and tumor associated antigens would induce a predictably heightened tumor specific response by the host. This prediction was borne out by the Applicant's work showing that SAg transfection of tumor cells abolished the tumorigenicity of 4T1 mammary carcinoma cells, significantly reduced the number of established metastases and prolonged survival compared to untreated controls. (Pulaski, Terman, et al., American Association of Cancer Research, April 1999 and submitted to Proc. Natl.Acad. Sci, 1999).

SAg transfected tumor cells in vivo are effective in an additional manner which does not apply to SAg polypeptide. Ingestion of apoptotic cells by DCs augments the immunogenicity of tumor cells. Fields et al., Proc. Natl. Acad. Sci., 95: 9882-9887, (1998); Albert et al., Nature, 392: 86-89, (1998). DCs are acknowledged as the premier accessory cell for antigen presentation. They have been shown to ingest apoptotic cells and nucleic acids and process them for presentation to host T cells in the context of costimulation, adhesion and MHC molecules. Akbari et al., J. Exp. Med., 189: 169-177, (1999). Therefore, following apoptosis of SAg transfected tumor cells and ingestion by DCs, SAg-encoding nucleic acid as well as tumor associated nucleic acids in the transfected cells would produce additional anti-tumor responses. Purified polypeptide enterotoxins do not share with the SAg transfectants this property of enhanced immunogenicity following ingestion and processing by DCs.

There are enormous structural and functional differences between the polypeptide enterotoxin and SAg-transfected tumor cells. The starting materials are different i.e. peptides vs nucleic acids and the product is different i.e. polypeptide vs enterotoxin transfected cell in which the SAg is may exist in nucleic acid and peptide form associated with a vast number of intracellular and membrane structures. Some of these structures may actually improve the T cell activating function of SAgs such as deoxyribonucleic acids, ribonucleic acids, tumor associated antigens, heat shock proteins, costimulatory molecules and adhesion molecules and endosomes. Cellular SAg peptides or nucleotides exist in association with tumor associated antigens, costimulants, adhesion molecules, heat shock proteins and MHC molecules, GPI-ceramides or SAg receptors (digalactosylceramides) which improve the immunogenicity of the tumor antigens. Therefore, these structural and functional differences between the polypeptide SAg and the enterotoxin transfected tumor cells clearly show that SAg transfected tumor cells have a far greater potential than the polypeptide to induce a tumor specific response.

Moreover, SAg transfected tumor cells possess an additional unique property not shared by the polypeptide SAg. SAg-transfected tumor cells display the metastatic phenotype of the tumor cells which enables them to colonize and traffic to metastatic sites in vivo. Once localized to micrometastatic sites the transfectants expressing SAg induce a potent tumor specific T cell response. In contrast, the purified polypeptide SAg unassociated with a tumor cell would have no capacity whatsoever to colonize metastatic sites.

The present invention also provides SAg-encoding nucleic acid, preferably DNA, fused with (or cotransfected with ) a nucleic acid encoding another molecule. The transfected cells include tumor cells, accessory cells e.g., DCs, tumor cell/accessory cell (e.g., DC) hybrids. The expression of molecules in addition to enterotoxins by these cells serves the following functions:

1) enhance the immunogenicity of the SAg transfected cell by providing nucleic acids encoding an additional potent immunogen. Examples would include tumor associated antigens or mutated normal antigen or fusion peptides in tumor cells, an immunogenic bacterial product such as Staphylococcal adhesin protein A, LPS, β-glucans, and peptidoglycans, costimulatory and adhesion molecules, heat shock protein, growth factor receptors such as Her/neu and tumor markers such as PSA.

2) assist in tumor killing activity by the SAg transfected cell when localized to tumor sites. by providing nucleic acids encoding the following: angiogenesis antagonists, chemoattractants such as C5a, chemokines such as RANTES, hyaluronidase and coagulase and CD44 isoforms.

3) increase the binding of immunogenic substances to the surface of the SAg transfected cell by providing nucleic acids encoding the following: CD1 receptors, CD14 receptors, SAg receptors

4) increase the production of SAg in the SAg transfected cell by providing nucleic acids encoding the following: cell cycle proteins, amplified oncogenes, and signal transduction molecules.

5) assist in trafficking of SAg to class I or class II pathway in the SAg transfected cell by providing nucleic acid encoding the following: the invariant chain, the LAMP1 proteins and TAP proteins.

6) induction of a local tumoricidal response by intratumoral injection of nucleic acids encoding the following: oxyLDL receptor and SAg receptor, chemoattractants, chemokines.

The present invention also provides for augmented tumoricidal responses by immunocytes particularly T, NK, and NKT cells. Inhibitory receptors or their tyrosine-based inhibitory motifs on T, NK, and NKT cells with specificity for lipid-based tumor associated antigens (LBTAAs) are deleted or functionally deactivated (antisense or gene knockout) which permits unopposed intacellular signaling by the activation receptors and enhanced responsiveness to LBTAAs and their respective tumors of origin. Inhibition of inhibitory receptor phosphorylases (SHP or SHIP) and/or ITIM binding sites on activation receptors (ITAM) is also contemplated as a means of augmenting the host response to LBTAAs.

### SUMMARY OF THE INVENTION

The present invention comprises a method for treating cancer in a host comprising providing conjugates, fusion proteins or naked nucleic acids of superantigen and additonal molecule(s) which produce an tumoricidal response. The addtional molecule serves the following functions: 1) to target a receptor (digalactosylceramide) expressed on tumor cells in vivo and induce tumor cell apoptosis e.g., SAg-verotoxin conjugates. 2) to target receptors expressed on tumor sinusoidal endothelium, induce apoptosis and a prothrombotic state e.g. SAg-oxyLDL conjugates and SAg-Lp(a) conjugates 3) to activate a dormant population of tumoricidal NKT cells e.g. SAg-digalactosylceramides, SAg-GPI-digalactosylceramide (phytosphingosine) complexes. 4) target receptors for integrins expressed on tumor microvasculature e.g., SAg-RGD conjugates. 5) naked DNA administered intratumorally induces tumor cell expresson in vivo of receptors for ligands which produce apoptosis and inflammation e.g, naked DNA SAg-oxyLDL receptor, SAg-LOX-1 receptor, SAg-SREC receptor.

Sickled erythrocytes are useful in the present invention since they have natural ligands for integrins expressed on tumor neovasculature which facilitates their targeting to the tumor endothelium. Sickled erythrocyte membranes acquire oxyLDL using fusigenic techniques with oxyLDL containing liposomes and apoproteins via gene transfection in the nucleated pre-reticulocyte phase. The oxyLDL and apoproteins expressed by the sickled cells facilitates targeting to oxyLDL, LOX-1 and SREC receptors present on the tumor microvasculature. These erythrocytes are also useful for carrying nucleic acids for transfection of the tumor endothelial cells in vivo. Vesicles derived from sickled erythrocytes are more rigid, prothrombotic and target the tumor microvascularture more effectively than the parent cell. They also carry oxyLDL to receptors on tumor endothelium. Likewise, vesicles, exosomes or SAg-GPI-digalctosylceramides shed from from SAg transfected tumor cells are capable of inducing potent tumoricidal responses and are useful in the present invention.

In addition, bacterial and yeast expression and phage display systems are useful for the presentation of SAg in association with other anti-tumor molecules. The yeast sec mutant or yeast display is used to produce a SAg-ceramide conjugate exhibiting a phytosphingosine in the sphingosine portion of the ceramide. This structure activates both T cell, NK cell and NKT cells. Sphingomonas paucimobilis which naturally expresses a-galactosylceramide is transfected with SAg nucleic acids which results in the shedding of SAg-a-galactosylceramide complexes which are use to produce a population of tumoricial T cells, NK cells and NKT cells. SAg phage displays with tumor localizing molecules e.g. RGD sequences are used to target SAgs to tumor microvasulature. SAg phage displays with similar tumor localizing molecules comprising tumor or tumor endothelial cell apoptosis inducing agents e.g., thrombospondin or oxyLDL are use to increase the tumoricidal response.

The present invention comprises a method for treating cancer in a host comprising providing cells transfected with a gene that express and/or secretes a SAg or T cells activated by the transfected cells to the host. The cells are transfected in vivo or in vitro. SAgs may activate T cells or NKT cells in the host. These same transfectants may be used to stimulate a population of T cells or NKT cells ex vivo which are provided to the host as tumor specific effector cells in adoptive immunotherapy. The transfected cells may be, for example, tumor cells accessory cells, DCs muscle cells, immunocytes, fibroblasts. When transfected in vitro the cells can be xenogeneic to the host, from the same species as the host or host cells.

For in vivo immunization, tumor cells are transfected with nucleic acids encoding SAgs together with a carbohydrate modifying enzyme such as galactosyl transferase to produce the Gal epitope, Staphylococcal hyaluronidase, Streptococcal capsular polysaccharide, Staphylococcal erythrogenic toxin, Staphylococcal Protein A, Staphylococcal b hemolysin, Staphylococcal coagulase, costimulants such as B7-1 and B7.2, chemoattractants and chemokines. SAgs are also cotransfected into tumor cells with gene clusters encoding the biosynthesis of highly immunogenic microbial Lipid A, membrane or capsular polysaccharides, lipoproteins and peptidoglycans. Nucleic acids are useful when transfected alone. However combinations are preferred. The cotransfection into tumor cells of the SAg-encoding nucleic acid together with the nucleic acids encoding Gal or GalCer biosynthesis is particularly useful. The cotransfection into tumor cells of the nucleic acid encoding SAg with nucleic acids encoding Staphylococcal erythrogenic toxins and hyaluronidase allows the transfected tumor cells to simulate the in vivo inflammatory activity of a Staphylococcus or leukocyte or macrophage by secreting enzymes and toxins which induce a sterile cellulitis in tumor sites.

Further provided are tumor cells transfected with nucleic acid encoding structures such as the erb/Neu gene which upon administration to the host promotes tumor cell trafficking and colonization of micrometastatic sites. Amplified oncogenes linked to SAg nucleic acids provide the locus and energy for expression or overexpression of both gene products. Thus, provided herein are tumor cells transfected with SAg-encoding nucleic acid together with nucleic acid encoding other oncogenes, amplified oncogenes and transcription factors, angiogenic factors such as angiostatin, angiogenesis receptors such as VEGF, tumor growth factors, tumor suppressors, cell cycle proteins and key proteins engaged in the antigen routing and processing pathway. In one example, the microbial SAg and erb/Neu nucleic acids are cotransfected into tumor cells. These nucleic acids may also linked to an inducible gene such as that encoding metallothionein or corticosteroid receptors. In this way, the cells are activated by exogenous delivery of corticosteroids or a heavy metal only after a suitable period of time has lapsed to allow them to localize in metastatic sites in vivo

Tumor cell transfectants are also useful ex vivo to immunize a T cell or NKT cell population producing tumor specific effector cell population for adoptive immunotherapy of cancer. These immunizing tumor cells are transfected with nucleic acids encoding SAgs and the SAg receptor. The latter transfectants are capable of binding exogenous SAg for presentation to a T cell population. In addition, tumor cells are transfected with nucleic acids encoding CD1 receptors which are capable of binding exogenous glycosylceramides and lipoarabinans free or bound to SAgs for presentations to T or NKT cells. Similarly, tumor cells are transfected with nucleic acids encoding the CD14 receptor which bind exogenous peptidoglycans and LPS's, free or bound to SAgs for presentation to T cells.

Likewise, the nucleic acids encoding the mannose receptor are transfected into tumor cells which are capable of binding a broad range of glycosylated SAgs for presentation to T cells. The present invention provides detailed methods for preparation of the SAg-glycosylceramide, SAg-LPS, SAg-peptidoglycan complexes as well as glycosylated SAgs which are loaded onto their respective receptors expressed on tumor cells, accessory cells and, in some instances, immunocytes. For ex vivo use, any prokaryotic or eukaryotic cell may be used which is transfectable with nucleic acid encoding SAgs to provide surface expression of the SAg or constructs expressed on tumor, accessory cell or immunocyte transfectants. When the transfected cells are not host tumor cells, the cells additionally express a tumor associated antigen expected to be present on the host's cancer cells.

Also provided is a tumor specific T cell, NK or NKT cell (collectively immunocyte) population which is activated by SAgs, SAgs conjugates given above or the tumor cell transfectants given above to produce a population of tumor specific effector cells useful in adoptive immunotherapy. A particularly effective method of producing a hyperresponsive immunocyte population is to delete (e.g. gene knockout) or inactivate (e.g. antisense) receptors on immunocytes or their respective immune receptor tyrosine-based inhibitory motifs (ITIMS) which inhibit cellular activation by receptors specific for lipid-based tumor associated (LBTAAs) and/or superantigens. After exposure to LBTAAs and/or superantigens, the immunocyte activation receptor response is unopposed by an inhibitory signal in which case immunocytes readily differentiate into tumor specific effector cells which are highly reactive even to weak LBTAAs.

After ex vivo stimulation, the T cells or NKT cells used for adoptive immunotherapy At should preferentially express CD44 which indicates that they are capable of trafficking and homing to tumor sites. Additionally, the T cell population used for ex vivo immunization is engineered to overexpress the TCR variable Vβ and invariant Vα sites specific for SAg and glycosylceramide binding respectively and to produce IFN by exogenous delivery of corticosteroids or a heavy metal. A particularly useful population of therapeutic tumor specific effector T cells or NKT cells which demonstrates overexpressed CD44 together with Vβ variable and Va invariant regions and high IFN production. Also provided are methods for reactivating anergic T cells in cancer patients by transfecting nucleic acids encoding the SAg receptors to produce a T cell population which may now be stimulated with exogenous SAgs.

Compositions which mimic SAgs are used in place of native SAgs for in vivo administration in order to circumvent the problem of naturally occurring SAg-specific antibodies. The SAg mimics are largely comprised of nucleotides or oligonucleotide-peptide chimeric constructs which are specific for tumor cells expressing SAg receptors (via the nucleotide) while retaining their SAg specificity for the TCR (via the peptide). The class II binding site of the SAg may optionally be eliminated or mutated to minimize SAg peptide binding to MHC class II receptors in vivo. The molecule may be composed entirely of nucleotides for which there are no naturally occurring antibodies. In addition, carriers are provided for in vivo transfection of tumors by nucleic acids encoding SAgs or other nucleic acid constructs given in Table I. Phage displayed tumor neovasculature ligands may also carry nucleic acids encoding SAgs or other constructs.

The constructs and method are used to treat any solid tumor such as carcinoma, melanoma and sarcoma or cancer of hemopoietic origin, such as lymphomas and leukemias which may or may not form solid tumors.

Unless otherwise defined, all technical and scientific terms used herein have the same meaning as commonly understood by one of ordinary skill in the art of this invention. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description and from the claims.

### BRIEF DESCRIPTION OF THE DRAWINGS

FIG. 1. Schematic diagram of the cloning of the SEB gene into the pHβ Apr1-neo vector. The coding region of the SEB gene was amplified with PCR primers. The upstream primer (SEB1) has a SalI site at its 5′end and the downstream primer (SEB2), a BamHI site. Both the pHbβ Apr1-neo vector and the amplified SEB insert were digested with SalI and BamHI, ligated and transformed into XL1-Blue competent cells. The final construct was verified by restriction enzyme and sequence analyses.

FIG. 2. Cloning of the SEB gene into the pHβ-Apr1 neo vector. Clones 1-5 contained the SEB insert (coding region 801 bp) and the pH-Apr1 neo vector (10 kb). All DNA was digested with SalI and BamHI and electrophoresed on a 1% agarose gel in 1×TAE buffer.

FIG. 3. Alignment of the published SEB coding sequence (SEQ ID NO: 1) and the newly constructed SEB gene (SEQ ID NO: 2) in pHβ-Apr1 neo vector (Clone #2). Clone #2 was sequenced with 4 primers: SEB1, 2, 3, and 4. SEB1 and 2 (SEQ ID NOS: 3-4) are the PCR primers that were used for the amplification of the SEB gene. SEB 3 (SEW ID NO: 5(TATGAAAGTTTTGTATGATGAT) and SEB 4 (SEQ ID NO: 6) (AGTGACGAGTTAGGTAATCT) are internal primers. The final sequence was confirmed by the multiple overlapping of sequences and aligned with the published SEB sequence. It is a perfect match. The start codon (ATG) and the stop codon (TGA) are underlined. The upstream and the downstream sequences are the human β-actin promoter and the SV40 polyA sequences in the pHβ-Apr1 neo vector with the addition of SalI and BamHI restriction enzyme sites.

### TABLE I

Therapeutic Constructs and Preferred Conditions of Use

I. CELLS: Tumor Cells, DCs or DC/Tumor Cell Hybrids (DC/tc)

USE: In vivo and Ex vivo

Purpose

A. In Vivo Preventative or Therapeutic Vaccine (Established Tumor)

Accomplish by transfecting or co-transfecting with nucleic acid encoding superantigen plus one or more of the following:

1. Superantigens

2. Enzyme that modifies carbohydrate to induce Gal or GalCer epitope expression

3. Functional hyaluronidase from microbial or human sources

4. Staphylococcal or streptococcal erythrogenic toxin

5. Staphylococcal protein A or a domain thereof

6. Staphylococcal hemolysin and functional microbial toxins

7. Functional microbial or human coagulase

8. Costimulatory protein

9. Chemoattractants

10. Chemokines

11. Nucleic acids encoding biosynthesis of lipopolysaccharides

12. Nucleic acids encoding biosynthesis of glycosylceramides

13. Nucleic acids encoding biosynthesis of microbial membrane or capsular lipoproteins and polysaccharides

14. Oncogenes, amplified oncogenes and transcription factors

15. Angiogenic factors and receptors

16. Tumor growth factor receptors

17. Tumor suppressor receptors

18. Cell cycle proteins

19. Heat-shock proteins, ATPases and G proteins

20. Proteins engaged in antigen processing, sorting and intracellular trafficking

21. Inducible nitric oxide synthase (iNOS)

22. apolipoproteins (e,g,. Lp(a)) transfected into tumor cells & sickled erythrocytes used for targeting tumor microvasculature

23. LDL and oxyLDL receptors (e.g., SCEP receptor) transfected into tumor cells and sickled erythrocytes & used for targeting to tumor microvasculature

B. Ex Vivo Immunization of T and/or NKT cells to Produce Tumor Specific Effector Cells (for Adoptive Immunotherapy)*

Accomplish by (i) transfecting or co-transfecting tumor or accessory cells with nucleic acid encoding the following, or (ii) providing immobilized molecules or receptors that present the following:

1. Superantigen

2. Superantigen receptor and transcription factor with bound superantigen

3. CD1 receptor binding and/or expressing superantigen-glycosyl ceramide complex

4. CD14 receptor binding or expressing superantigen-lipopolysaccharide or superantigen-peptidoglycan complex

5. Mannose receptor binding glycosylated superantigen

6. Glycophorin receptor

7. Superantigen-tumor peptide(s) complex on MHC or CD1-bearing APC in soluble or immobilized form

C. Therapeutic Molecules or Complex Applied to Transfected or Untransfected Tumor cells or Accessory Cells, or MHC class I, class II, CD1 , Superantigen receptor or CD14 receptor:

1. Superantigen (wherein cell may express Gal)

2. Glycosylated superantigen

3. Superantigen complex with

a. glycosyl ceramide

b. lipopolysaccharide

c. peptidoglycan

d. mannan proteoglycan

e. muramic acid

f. tumor peptide

g. glycosylceramides with terminal Gal(α 1-4)Gal e.g globotriosylceramide and galabiosylceramide

h. Conjugates of SAg-(Gb2 or Gb3 or Gb4)

i. Conjugates of SAg-(Gb2 or Gb3 or Gb4)-CD1

j. GPI anchored conjugates: SAg-GPI-(Gb2 or Gb3 or Gb4)

l. GPI anchored conjugates: SAg-GPI-(Gb2 or Gb3 or Gb4)-CD1

m. Conjugates of SAg polypeptide or nucleic acid with Verotoxin

n. Conjugates of SAg Polypeptide or nucleic acid with Verotoxin A or B subunit

o. Conjugates of SAg polypeptide or nucleic acid with IFNα receptor peptides homologous to verotoxin

p. Conjugates of SAg polypeptide or nucleic acid with CD19 peptides homologous to verotoxin

q. Conjugates of SAg polypeptide or nucleic acid with Arg-Gly-Asp or Asn-Gly-Arg

r. Conjugates of SAg polypeptide or nucleic acid with LDL, VLDL, HDL

s. Conjugates of SAg polypeptide or nucleic acid with Apolipoproteins (e.g., Lp(a), apoB-100, apoB-48, apoE)

t. Conjugates of SAg polypeptide or nucleic acid with oxyLDL, oxyLDL mimics, (e.g., 7β-hydroperoxycholesterol, 7β-hydroxycholesterol, 7-ketocholesterol, 5α-6α-epoxycholesterol, 7β-hydroperoxy-choles-5-en-3β-ol, 4-hydroxynonenal (4-HNE), 9-HODE, 13-HODE and cholesterol-9-HODE)

u. Conjugates of SAg polypeptide or nucleic acid with oxyLDL byproducts (e.g. lysolecithin, lysophosphatidylcholine, malondialdehyde, 4-hydroxynonenal)

v. .LDL & oxyLDL receptors (e.g., LDL oxyLDL, acetyl-LDL, VLDL, LRP, CD36, SREC, LOX-1, macrophage scavenger receptors) as polypeptide or nucleic acid alone or with SAg polypeptide or nucleic acid intratumorally

w. phytosphingosine, -GPI-phytosphinosine, —

x. tumor associated lipid antigens glycolipid, proteolipid, glycosphingolipid, sphingolipid with inositolphosphate-containing head groups, phytoglycolipids, mycoglycolipids. -GPI-sphingosines or lipids

y. sphingolipids with inositolphosphate-containing head groups having the general structure: ceramide-P-myoinositol-X with X referring to polar substituents comprising ceramide-p-inositol-mannose, inositol-1-P-(6)mannose(α 1,2inositol-1P-(1) ceramide, (inositol-P)2-ceramide, inositol-P-inositol-P-ceramide, inositol-P-inositol-P-ceramide.

z tumor associated glycan antigens consisting of peptidoglycans or glycan phosphotidyinositol (GPI) structures.

II. CELLS: Specialized Tumor Specific Effector Cells (T and/or NKT Cells)

USE: Adoptive Immunotherapy In Vivo

Purpose:

A. CD44 Expression on T cells or NKT

Accomplished by: (i) Superantigen stimulation; and/or (ii) transfection with nucleic acid encoding CD44 and/or (iii) transfection with nucleic acid encoding glycosyltransferase

B. Chimeric TCR with:

Invariant a chain site for binding GalCer and

Vβ chain site for binding superantigen

C. Dual TCR Vβ chains with sites for superantigen binding

D. T cells or NKT cells with overexpressed Vβ region specific for a given superantigen

E. T cells or NKT cells with lowered signal transduction threshold

III. MOLECULES: Superantigen mimics

USE: In Vivo Administration

A. Superantigen receptor-binding oligonucleotides

B. Superantigen oligonucleotide-peptide conjugate

Oligo nucleotide is specific for superantigen receptor on tumor cells

Peptide has deleted class II binding site and intact TCR binding site

C. Phage displayed integrin ligand on tumor neovasculature—carrier for superantigen-encoding nucleic acid.

IV. CARRIERS: for nucleic acid encoding superantigen

USE Transfection of Tumors In vivo

A. Sickled erythrocytes that target tumor neovasculature

B. Phage displayed tumor neovascular integrin and superantigen receptor carrying superantigen nucleic acids

V. CARRIERS: constructed to co-express superantigen conjugates or complexes with:

Glycosylceramide

α-Gal

Lipopolysaccharides

Peptidoglycans

USE Transfection of Tumor Cells and/or DCs and/or DC/tc's—in vivo or ex vivo.

A. Liposomes

B. Proteosomes

### DESCRIPTION OF THE PREFERRED EMBODIMENTS

The present invention provides methods and materials for treating cancer related to the polypepide or nucleic acid conjugates or fusions comprising SAg with other molecules that synergize or cooperate with SAg in the induction of an anti-tumor response. The present invention also provides materials and methods for treating cancer related to transfection of cells with nucleic acid that encode a SAg and/or another polypeptide. The cells can be transfected in vivo or in vitro. The expression of the SAg polypeptide activates host immunocytes, such as T or NKT cells.

As used in this application, T cells are defined as any class of lymphocytes that undergo maturation and differentiation in the thymus. They include, but are not limited to NK cells, NKT cells and/T cells and may be known as cytotoxic, helper or suppressor T cells or they may be defined by the expression or type of CD or TCR present. The same transfected nucleic acid molecule, or a separate nucleic acid molecule, can also encode another polypeptide such as an adhesion molecule, glycosyltransferase, glycosidase, CD44, cytokine, tumor associated antigen, costimulatory molecule, and the like. In addition, cells transfected in vitro or ex vivo with any of these nucleic acids as well as T cells activated by these transfected cells are administered directly to a cancer-bearing host. Cells transfected in vitro or ex vivo as well as cells activated ex vivo may additionally express a tumor associated antigen expected to be present on host cancer cells. Further, cells transfected with nucleic acid that encodes a SAg polypeptide is also be used as a vaccine to immunize a host against a cancer previously present in the host or a cancer that is likely to develop in the host. For example, a host can be vaccinated against a particular cancer by administering tumor cells transfected with nucleic acid encoding a SAg. Alternatively, a SAg transfected cell is used to activate a host T cell population in vitro. This activated T cell population is then administered to a host as a cancer treatment (immunotherapeutic agent). Once activated ex vivo or in vivo, these T cells are expanded with cytokine treatment such as IL-2 treatment.

Cells to be “transfected” include accessory cells, immunocytes, fibroblasts, or tumor cells. Accessory cells may include, without limitation, endothelial cells, DCs, monocytes, macrophages as well as B and T lymphocytes which can play an “accessory” as well as direct effector role in an immune response. When transfected in vitro, the cells can be xenogeneic, allogeneic to the host to provide, among other things, additional immunogenicity. Preferably, the transfected cells that are administered to a host, preferably a human, are syngeneic or autologous (or autochthonous).

Cells transfected with nucleic acid encoding a SAg may also express a tumor associated antigen that is potentially present on host cancer cells. For example, nucleic acid encoding a known tumor antigen are transfected into the SAg-containing cell, or a tumor cell that endogenously contains many different tumor antigens are transfected with SAg-encoding nucleic acid. In the latter case, additional nucleic acids encoding other polypeptides are transfected into the tumor cell. For example, nucleic acid encoding a carbohydrate modifying enzyme such as α 1,3-galactosyltransferase, adhesion molecule, costimulatory molecule such as B7-1 and B7-2, MHC class I molecule and/or MHC class II molecule are cotransfected into tumor cells together with nucleic acid encoding a SAg. SAg-encoding nucleic acid can encode a mutant, variant, and/or modified form of a SAg. These forms can be used to transfect T cells, alone or in combination with wild-type SAg-encoding acid.

In addition, tumor cells are provided with the capacity to colonize sites of metastases and the ability to locally hydrolyze surrounding tumor ground substance and neovasculature by transfection of key bacterial Staphylococcal and Streptococcal enzymes, toxins and capsular polysaccharides which confer upon the tumor cell additional tumor killing properties and immunogenicity. The transfected genes include staphylococcal hyaluronidase (tissue spreading factor), Staphylococcal erythrogenic toxin and Streptococcal capsular polysaccharide. The tumor cell may thus be capable of mimicking the tissue invasive and destructive properties of the Streptococcus and Staphylococcus as they produce a sterile cellulitis localized to tumor sites.

These methods are used to treat any solid tumor such as carcinoma, melanoma, and sarcoma, or cancers of hematopoietic origin such as leukemia and lymphomas. This invention also provides for T cells or NKT cells including γ/δ T cells which after activation by SAgs in native or mutant form or transfected into tumor cells express surface phenotypes which enhance their ability to traffic efficiently to tumor sites in vivo. Such phenotypes include CD44 and/or selective Vβ expression. In response to these SAg stimulants, the T cells produce TH1 cytokines and, in particular, IFNγand IL-2.

Further, provided are methods of overcoming the T cell unresponsiveness of cancer patients by transfection of T cells from tumor bearing host with the nucleic acids encoding the SAg receptor thus enabling these cells to be reactivated by exogenous SAg and used for adoptive immunotherapy in the same cancer patient. Provided herein are SAg oligonucleotide and oligonucleotide-peptide compositions capable of targeting and delivering SAgs to tumor sites in vivo without elimination by circulating naturally occurring SAg specific antibodies prevalent in the human cancer patients. Provided also are compositions and methods for delivery of therapeutic nucleic acid constructs to tumor sites in vivo using therapeutic genes carried by erythrocytes from patients with sickle cell anemia which have the unique capability of adhering to sites on tumor neovasculature.

1. Cancer

This invention is used to treat any type of cancer in a host at any stage of the disease. More particularly, the cancer is a solid tumor such as a carcinoma, melanoma, or sarcoma. This invention is used to treat cancers of hemopoietic origin such as leukemia or lymphoma, that involve solid tumors. A host is any animal that develops cancer and has an immune system such as mammals. Thus, humans are considered hosts within the scope of the invention.

Since the invention provides SAg-transfected cells as a vaccine, a cancer is one that a host is likely to develop based on family history or other criteria. In this case, the host is one that is susceptible to cancer.

2. Nucleic Acid

The term nucleic acid as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand.

The term isolated nucleic acid means that the nucleic acid is not immediately contiguous with both of the sequences with which it is immediately contiguous (one on the 5′ end and one on the 3′ end) in the naturally occurring genome of the organism from which it is derived. For example, an isolated nucleic acid molecule can be, without limitation, a recombinant DNA molecule of any length, provided nucleic acid sequences normally found immediately flanking that recombinant DNA molecule in a naturally occurring genome are removed or absent. Thus, an isolated nucleic acid molecule includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

Typically, regulatory elements are nucleic acid sequences that regulate the expression of other nucleic acid sequences at the level of transcription and/or translation. Thus, regulatory elements include, without limitation, promoters, operators, enhancers, ribosome binding sites, transcription termination sequences (i.e., a polyadenylation signal), and the like. In addition, regulatory elements can be, without limitation, synthetic DNA, genomic DNA, intron DNA, exon DNA, and naturally-occurring DNA as well as non-naturally-occurring DNA. It is noted that isolated nucleic acid molecules containing a regulatory element are not required to be DNA even though regulatory elements are typically DNA sequences. For example, nucleic acid molecules other than DNA, such as RNA or RNA/DNA hybrids, that produce or contain a DNA regulatory element are considered regulatory elements. Thus, recombinant retroviruses having an RNA sequence that produces a regulatory element upon synthesis into DNA by reverse transcriptase are isolated nucleic acid molecules containing a regulatory element even though the recombinant retrovirus does not contain any DNA.

3. Transfection

The term “transfection,” of a nucleic acid into a cell, as used herein is intended to include “transformation,” “transduction,” “gene transfer” and the like, as they are commonly used in the art. “Transfection” is not intended to be limited to transfer of nucleic acid into a cell by means of an infectious particle such as a retrovirus, as the term may have been used originally. Rather any form of delivery and introduction of a nucleic acid molecule, preferably DNA, into a cell, whether in the form of a plasmid, a virus, a liposome-based vector, or any other vector, so that the nucleic acid is expressed in the cell and its protein product(s) made, is included within the definition of “transfection.”

When a nucleic acid is said to “encode” a product other than a protein, this language is intended to mean that it encodes the necessary proteins/enzymes that are involved in, or required for, the synthesis of that product. For example, if a DNA molecule is said to encode LPS, it clearly encodes one or more proteins (enzymes) that are involved in the biosynthesis of LPS. If a nucleic acid is said to “encode the biosynthesis” of a structure, it means that the nucleic acid encodes the enzymes that participate in the creation of that structure. In particular for the carbohydrate structures referred to herein, the nucleic acids used in the invention are introduced into a cell that normally does not make, or makes little of, the carbohydrate structure so as to provide to that cell the genetic material for an enzyme or enzymes that generate the carbohydrate structure or modify a different carbohydrate structure to that one indicated. As a further example, DNA encoding a tumor antigen may directly encode a protein/peptide tumor antigen, or alternatively, may encode proteins or peptides that either contribute structurally to, or catalyze the synthesis of, a tumor antigen which is partly protein (e.g., lipoprotein or proteoglycan) or totally non-protein (e.g., a glycolipid).

The invention provides methods of treating cancer in a host by transfecting cells with SAg-encoding nucleic acid. Suitable host or non-host cells for transfection include, without limitation, endothelial cells, DCs, monocytes, macrophages, B cells, T cells, immunocytes, muscle cells, fibroblasts, NK cells, NKT cells (TCR ab⁺ CD4^{neg} and CD8^{neg}), γ/δ T cells and tumor cells. The terms accessory cell and antigen presenting cell (APC) can be used interchangeably and include cells having the ability to process and present antigens to T cells as well as to provide both defined and less well defined growth and differentiation factors (costimulatory signals) during an ongoing immune response.

Cells are transfected in vivo or in vitro. When transfected in vivo, the cells are of host origin. When transfected in vitro, the cells are autologous, allogeneic, or xenogeneic to the host to provide additional immunogenicity. In addition to being transfected with nucleic acid encoding a SAg, the cells are transfected with nucleic acid encoding any other polypeptide including, without limitation, α-galactosyltransferase, staphylococcal hyaluronidase and/or erythrogenic toxin, streptococcal capsular polysaccharide, CD44, tumor antigen, costimulatory molecule such as B7-1 and B7-2, adhesion molecules, MHC class I molecule and/or MHC class II molecule. Nucleic acids encoding the molecules are cotransfected with the SAgs. But for others, including but not limited to Staphylococcal hyaluronidase, erythrogenic toxin, Streptococcal capsular polysaccharide and CD44 genes, the nucleic acids encoding the SAgs are fused to other nucleic acids resulting in expression of a fusion protein. Methods for in vivo and in vitro transfection of cells are well known. For example, two books in the series Methods in Molecular Medicine published by Humana Press, Totowa, N.J., describe in vivo and in vitro transfection protocols that are adaptable to the present invention (Vaccine Protocols edited by Robinson et al., (1996) in Gene Therapy Protocols edited by Robbins et al., Humana Press, Totowa, N.J. (1997)). Transfection protocols are also discussed elsewhere ((Sambrook, J. et al., Molecular Cloning, Second Edition, Cold Springs Harbor Laboratory Press, Plainview, N.Y., (1989)). In addition, use of various vectors to target epithelial cells, use of liposomal constructs, methods of transferring nucleic acid directly into T cells, hematopoietic stem cells, and fibroblasts, methods of particle-mediated nucleic acid transfer to skin cells, and methods of liposome-mediated nucleic acid transfer to tumor cells have been described elsewhere. (Felgner, P L et al., Cationic Lipids for Intracellular Delivery of Biologically Active Molecules, U.S. Pat. No. 5,459,127, issued Oct. 17, 1995; Felgner, P L, Cationic Lipids for Intracellular Delivery of Biologically Active Molecules, U.S. Pat. No. 5,264,618, issued Nov. 23, 1993; Felgner, P L, Exogenous DNA Sequences in a Mammal, U.S. Pat. No. 5,580,859 issued Dec. 3, 1996; Felgner, P L, A Protective Immune Response in a Mammal by Injecting a DNA Sequence, U.S. Pat. No. 5,589,466 issued Dec. 31, 1996).

Further, use of ligand-based nucleic acid carriers to effect transfection of mammalian cells in vivo has been described elsewhere (Wu et al., J. Biol. Chem., 262:4429-4432 (1987); Wu et al., J. Biol. Chem., 263:14621-14624 (1988); Wu et al., J. Biol. Chem., 264:16 985-16987 (1989); Wu et al., J. Biol. Chem., 266:14338-14342 (1991); and Garrigues J et al., Am. J. Path. 142:607-622 (1993)). Briefly, plasmid DNA is conjugated to a desialylated branched carbohydrates such as orosomucoid by carbodiimide crosslinking to polylysine and targeted to asialoprotein receptors expressed on cells in the liver. In addition, enhanced nucleic acid delivery and expression can be achieved using a ligand-polylysine conjugate coupled to carbohydrate moieties on viruses that is then combined with DNA. These preparations are suitable for parenteral injection and are readily taken up by cells expressing asialoprotein receptors in the liver after which the DNA is internalized and integrated into the host genome. In addition, nucleic acid can be administered intravenously, intramuscularly, or subcutaneously to induce a response in a host.

Thus, targeting nucleic acid to the surface of particular cells is accomplished by conjugating nucleic acid to molecules that bind to a cell surface structure such as a receptor. Examples of cell surface structures that can be targeted include, without limitation, the transferrin receptor, and asialoglycoprotein receptor. The molecules that bind cell surface structures and are conjugated to nucleic acid for targeting can be, without limitation, natural ligands for the surface structure, synthetic compositions that exhibit specific binding, and antibodies directed against the surface structure. For example, a monoclonal antibody specific for a cell surface epitope such as the BR96 antibody that recognizes Le^{x} carbohydrate epitope abundantly expressed by colon, breast, ovary, and lung carcinomas can be used. Other monoclonal antibodies can include, without limitation, those that recognize growth factor receptors, transferrin receptors, IL-2 receptors, epidermal growth factor receptors, the hev oncogene, and TAPA-1 as well as any other antibody having specificity for a surface structure that can be internalized.

Liposomes containing nucleic acid are also targeted to specific cell types such that the nucleic acid is expressed. For example, nucleic acid is loaded into or attached to cationic DOTMA: doleoylphosphatidylethanolamine (DOPE) liposomes that contain exposed molecules that bind to a cell surface structure such as tumor cells or tumor microvasculature (Example 5). The molecules that bind cell surface structures and are attached to liposomes can be, without limitation, natural ligands for the surface structure, synthetic compositions that exhibit specific binding, and antibodies directed against the surface structure. Maximal transfer of nucleic acids encoding SAgs is attained by synthesizing the liposomes with an appropriate ratio of nucleic acid to lipid. In addition, these nucleic acid-containing liposomes are administered intravenously, intramuscularly, or subcutaneously to induce a response in a host.

Naked nucleic acid is also administered to a host. For example, naked pharmaceutical-grade plasmid DNA are injected into a host intramuscularly such that it is expressed by host cells (U.S. Pat. Nos. 5,589,466; 5,580,599; 5,264,618; 5,459,127; and 5,561,064). In addition, cationic lipids are used to deliver biologically active molecules, such as oligonucleotides to host cells in vivo (U.S. Pat. Nos. 5,264,618, 5,459,127, and 5,561,064). Thus, nucleic acid encoding a SAg is administered to a host in naked or cationic lipid form such that the SAg is expressed. It is noted that any nucleic acid described herein can be administered in vivo as naked DNA. Further, other methods of administering naked DNA to a host can be used such as those related to the direct injection of naked DNA for use in vaccines (Cohen et al., Science 259:1691-1692 (1993); Corr et al., J. Exp. Med. 184:1555-1560 (1996); Varmus et al., Proc. Natl. Acad. Sci. USA 81:5849-5852 (1984); and Benveniste et al., Proc. Natl. Acad. Sci. USA 83:9551-9555 (1986)).

Our previous patent applications which are hereby incorporated by reference include U.S. patent applications Ser. No. 07/416,530, filed Oct. 3, 1989, U.S. patent application Ser. No. 07/466,577, filed Jan. 17, 1990, U.S. patent application Ser. No. 07891,718, filed Jun. 1, 1992, U.S. patent application Ser. No. 08/025,144, filed Mar. 2, 1993, U.S. patent application Ser. No. 08/189,424, filed Jan. 31, 1994, U.S. patent application Ser. No. 08/491,746, filed Jun. 19, 1995, PCT applications PCT/US91/00342, and PCT/US94/02339. These applications have given comprehensive description of the SAg genes, the creation of high enterotoxin producing mutant strains as well as recombinant methods of production of SAgs. In addition, methods of treating cancer by transfecting tumor cells in vivo and in vitro with SAg nucleotides using well defined recombinant technology have been described in these applications. Subsequently, Dow et al., (J. Clin. Invest. 99: 2616-2624 (1997)) described in vitro and in vivo transfection of eukaryotic cells with SAg DNA which was capable of inducing inflammatory responses in vivo. It is noted that the SAg genes have been cloned and their sequences delineated before 1988 and methods used to transfect cells in vivo or in vitro with nucleic acids encoding polypeptides are also well known in the art.

4. Constructs

Tumor cells are transfected with various nucleic acids which are designed to increase their immunogenicity and to provide them with capacity to traffic to metastatic sites where they may initiate a potent inflammatory and immune response. Such constructs of this invention can be linear or circular nucleic acids obtained from mammals or bacteria that encode a polypeptide such as a SAg, mutant SAg, erythrogenic toxin, enzymes involved in the biosynthesis of glycosyltransferases, bacterial glycosylcerarnides, LPS's, lipoproteins, capsular or membrane polysaccharides, microbial toxins and enzymes such as hyaluronidase, collagenase, elastase, coagulase, protease, kinase, lipase. Constructs may also contain tumor associated antigens, costimulatory molecules such as B7-1 and B7-2, adhesion molecules, receptor molecules such as SAg receptors, CD1, CD14, MHC class I molecules and/or MHC class II receptors. Such constructs may also contain amplified nucleic acids associated with tumors such as oncogenes, transcription factors, angiogenesis factors and receptors, tumor growth factor receptors, chimeric receptors. The latter nucleic acids may be linked to SAg-encoding nucleic acid to produce heightened expression of the SAg. The amplified nucleic acids may include tumor tissue specific promoters and nucleic acids that direct the colonization or metastasis of tumors to selected sites in vivo.

Constructs can also contain elements that regulate and/or promote the expression of an encoded polypeptide. For example, a construct containing nucleic acid that encodes enterotoxin B (SEB) can have a strong promoter element upstream of the SEB encoding sequence. In addition, constructs can contain nucleic acid that anchors an encoded polypeptide to the cell surface after expression. For example, a construct containing nucleic acid that encodes SEB can contain a membrane-anchoring sequence such as nucleic acid that encodes a hydrophobic stretch of amino acids or a glycosylphosphatidylinositol (GPI)-anchoring motif Thus, the SAg, or other polypeptides as well, can be anchored in the plasma membrane by coupling to membrane lipids or glycolipids. These anchors can be attached to the C terminus of the polypeptide in the endoplasmic reticulum. Alternatively, a SAg known to be associated with the cell surface after expression can be used such as the mammary tumor viral (MMTV) SAg that is GPI-linked.

In one embodiment, SAgs as well as SAg receptors are engineered to remain anchored to the surface of transfected cells when the cell is to be used for immunization. Likewise, when a SAg receptor gene is transfected into anergized T cells from cancer patients, it is desirable to express the receptor on the cell surface so that they are readily recognized and activated by exogenous receptor bound SAg. In contrast, when it is desirable to use SAg transfected cells to activate T cells in vivo or ex vivo or to promote trafficking of transfected tumor cells to metastatic sites in vivo, it is suitable for the SAg to be secreted from the transfected cells.

In additional embodiments, potent tumor specific effector T or NKT cell clones are produced with overexpressed Vβ regions of their TCRs making them highly receptive to activation by exogenous SAg. Likewise CD44 genes are transfected into T cells or NKT cells making them more susceptible to expression of this epitope after SAg stimulation.

Constructs also contain a selectable marker or reporter such that transfected cells can be isolated. For example, a construct containing nucleic acid that encodes a SAg can also contain nucleic acid that encodes a polypeptide that confers resistance to a selection agent such as neomycin (also called G418), puromycin, or kanamycin.

Nucleic acid and nucleic acid constructs of the present invention are incorporated into a vector, an autonomously replicating plasmid, or a virus (e.g., a retrovirus, adenovirus, or herpes virus). Typically, these vectors, plasmids, and viruses can replicate and function independently of the cell genome or integrate into the genome. Vector, plasmid, and virus design depends on, for example, the intended use as well as the type of cell transfected. Appropriate design of a vector, plasmid, or virus for a particular use and cell type is within the level of skill in the art. In addition, a single vector, plasmid, or virus can be used to express either a single polypeptide or multiple polypeptides. It follows that a vector, plasmid, or virus that is intended to express multiple polypeptides will contain one or more operably linked regulatory elements capable of effecting and/or enhancing the expression of each encoded polypeptide.

The term “operably linked” means that two nucleic acid sequences are in a functional relationship with one another. For example, a promoter (or enhancer) is operably linked to a coding sequence if it effects (or enhances) the transcription of the coding sequence. A ribosome binding site is operably linked to a coding sequence if it is positioned to facilitate translation. Operably linked nucleic acid sequences are often contiguous, but this is not a requirement. For example, enhancers need not be contiguous with a coding sequence to enhance transcription of the coding sequence.

A vector, plasmid, or virus that directs the expression of a polypeptide such as a SAg can include other nucleic acid sequences such as, for example, nucleic acid sequences that encode a signal sequence or an amplifiable gene. Signal sequences are well known in the art and can be selected and operatively linked to a polypeptide encoding sequence such that the signal sequence directs the secretion of the polypeptide from a cell. An amplifiable gene (e.g., the dihydrofolate reductase [DHFR] gene) in an expression vector can allow for selection of host cells containing multiple copies of the transfected nucleic acid.

Standard molecular biology techniques are used to construct, propagate, and express the nucleic acid, nucleic acid constructs, vectors, plasmids, and viruses of the invention ((Sambrook, J.et al., supra; Maniatis et al., Molecular Cloning_(1988); and U.S. Pat. No. 5,364,934. For example, prokaryotic cells (e.g., E. coli, Bacillus, Pseudomonas, and other bacteria), yeast, fungal cells, insect cells, plant cells, phage, and higher eukaryotic cells such as Chinese hamster ovary cells, COS cells, and other mammalian cells can be used.

Constructs are used in vivo or ex vivo or in combination as in Example 5-7, 16-23. They are used to immunize a host by direct in vivo administration or they are used ex vivo to activate T cells or NKT cells to become tumor specific effector cells which are employed for adoptive immunotherapy of cancer by methods and models (Examples 7, 16, 19-23).

To test the anti-tumor-inducing ability of a particular construct as well as the transfected cell itself, the following general assay is performed. B16 melanoma, A20 lymphoma, host tumor cells, or any other tumor cell lines appropriate to the host (i.e., having tumor antigens expected to be present on the host tumor cells) are transfected with a given construct. Appropriate numbers of transfected cells (e.g., 10⁵-10⁷ cells) are then implanted subcutaneously into animals such as mice, rats, rabbits, or the like and 1-6 months later untransfected tumor cells are implanted. Tumor outgrowth from the untransfected tumor cells is measured and compared to control animals not given the transfected tumor cells. If tumor outgrowth is reduced or prevented, then the transfected cells are effective anti-tumor agents useful as tumor vaccines. Alternatively, 10⁵-10⁷ transfected tumor cells can be given 3-10 days after the appearance of established tumors from untransfected tumor cells. If tumor outgrowth is reduced or arrested, then the transfected cells are effective anti-tumor agents useful in treating established tumors.

To test the anti-tumor effect of SAg activated T cells, NKT cells or T cells clones overexpressing Vβ or CD44, the following general protocol is used. Lymph node cells from C57/B1 mice bearing MCA 205 or 207 sarcomas which were implanted in the adjacent inguinal region three to ten days before are extracted and placed in tissue culture. The cells are incubated with various enterotoxins for two days and then with IL-2 for an additional two to three days. The cells are then harvested and injected into syngeneic mice with established pulmonary metastases (six to twelve days after tumor injection). Three weeks later the animals are evaluated for pulmonary metastases compared to controls which receive no cells or cells that were stimulated without enterotoxins. The adoptively transferred cells may be enriched for NKT cells or T cells alone (to include γ/δ T cells) which are selectively injected into tumor bearing hosts. Likewise, they are selected for predominant expression of the CD44 phenotype during the SAg activation phase at which time the CD44 enriched population is harvested and used for adoptive immunotherapy. The dose of injected T cells, NKT cells or γ/δ T cells and/or CD44 enriched cells (which are produced by any of these T cell, NKT cell or γ/δ T cell populations) range from 10⁶ to ⁷ and are be given on a schedule of once weekly for one to four weeks.

5. Superantigens (SAgs)

SAgs are polypeptides that have the ability to stimulate large subsets of T cells. SAgs include Staphylococcal enterotoxins, Streptococcal pyrogenic exotoxins, Mycoplasma antigens, rabies antigens, mycobacteria antigens, EB viral antigens, minor lymphocyte stimulating antigen, mammary tumor virus antigen, heat shock proteins, stress peptides, clostridial and toxoplasmosis antigens and the like. Any SAg can be used as described herein, although, Staphylococcal enterotoxins such as SEA, SEB, SEC, and SED and streptococcal pyrogenic exotoxins such as toxic shock-associated toxin (TSST-1 also called SEF) are preferred.

When using enterotoxins, the region related to emetic activity can be omitted to minimize toxicity. In addition, SAgs can be derivatized to minimize toxicity. The level of toxicity may not be a concern when using SAg transfected cells to activate lymphocytes ex vivo since the lymphocytes can be rinsed of SAg polypeptide prior to administration to a host.

The nucleic acid sequences that encode SAgs are known and readily available. For example, Staphylococcal enterotoxin A (SEA)(SEQ ID NOS: 7-8), SEB (SEQ ID NOS: 9-10), SEC (SEQ ID NOS: 11-12), SED (SEQ ID NOS: 13-14), SEE (SEQ ID NOS: 15-16), TSST-1 (SEQ ID NOS: 17-18), and Streptococcal pyrogenic exotoxin (SPEA) (SEQ ID NOS: 19-20) have been cloned and can be expressed in E. coli (Betley M J and J J Mekalonos, J. Bacteriol. 170:34 (1987); Huang I Y et al., J. Biol. Chem., 0 262:7006 (1987); Betley M et al., Proc. Natl. Acad. Sci USA, 81:5179 (1984); Gaskill M E and S A Khan, J. Biol. Chem., 263:6276 (1988); Jones C L and S A Khan, J. Bacteriol., 166:29 (1986); Huang I Y and M S Bergdoll, J. Biol. Chem., 245:3518 (1970); Ranelli D M et al., Proc. Nat. Acad. Sci. USA 82:5850 (1985); Bohach G A, Infect Immun., 55:428 (1987); Bohach G A, Mol. Gen. Genet. 209:15 (1987); Couch J L et al., J. Bacteriol. 170:2954 (1988); Kreiswierth B N et al., Nature, 305:709 (1983); Cooney J et al., J. Gen. Microbiol., 134:2179 (1988); Iandolo J J, Annu. Rev. Microbiol., 43:375 (1989); and U.S. Pat. No. 5,705,151)). Additional nucleic acid sequences encoding SAgs are described elsewhere (Bohach et al., Crit. Rev. in Microbiology 17:251-272 (1990); (Kotzin, B L et al., Advances Immunology 54: 99-165 (1993)) PCR can be used to isolate SAg-encoding acid. For example, the nucleic acid encoding SEA, SEB, and TSST-1 can be isolated as described elsewhere (Dow et al., J. Clin. Invest. 99:2616-2624 (1997)). Briefly, the following primers can be used to amplify the SAg-encoding nucleic acid:

SEA forward: (SEQ ID NO: 21) GGGAATTCCATGGAGAGTCAACCAG,

SEA backward: (SEQ ID NO: 22) GCAAGCTTAACTTGTTAATAG;

SEB forward: (SEQ ID NO: 23) GGGAATTCCATGG-AGAAAAGCG,

SEB backward: (SEQ ID NO: 24) GCGGATCCTCACTTTTTCTTTG; and

TSST-1 forward: (SEQ ID NO: 25)GGGGTACCCCGAAGGAGGAAAAAAAAATGTCTACAAACGATAATATA AAG,

TSST-1 backward: (SEQ ID NO: 26) TGCTCTAGAGCATTAATTAATTTCTGCTTCTATAGTTTTTAT.

The full-length TSST-1 nucleic acid sequence is cloned into a eukaryotic expression vector (pCR3; In Vitrogen Corp., San Diego, Calif.), whereas only the sequence corresponding to the mature SEB and SEA (sequences minus the putative bacterial signal sequences) is cloned into pCR3. Removal of the SEB and SEA signal sequences increases the level of expression in transfected cells. The plasmids are grown in Escherichia coli and plasmid DNA extracted by the modified alkaline lysis method and purified on a CsC1 gradient.

Nucleic acids encoding mutant or variant SAgs are also considered nucleic acid sequences encoding SAgs within the scope of the invention. For example, a mutant SAg-encoding acid sequence is engineered such that the resulting SAg is devoid of amino acid residues, e.g., histidine, known to produce toxicity. Likewise, SAg-encoding nucleic acid is engineered to contain or lack sequences that facilitate the selective binding of SAgs to certain Vβ regions of the TCR present on T cells or to ganglioside, mannose (or other carbohydrate) receptor, certain regions of MHC class II, and/or enterotoxin receptors present on tumor cells, antigen presenting cells (APCs), and/or lymphocytes.

Nucleic acid sequences that encode a SAg are also fused, in frame, with nucleic acid that encodes another polypeptide. This larger nucleic acid is termed herein a SAg fusion gene and the resulting polypeptide product is a SAg fusion product. Nucleic acid sequences that are fused to SAg-encoding nucleic acid include, without limitation, nucleic acid sequences that encode tumor antigens, costimulatory molecules, adhesion molecules and MHC class II molecules. The superantigen fusion product is secreted by a transfected cell, expressed on the cell surface or it may remain intracellular in nucleic acid or partly processed form.

SAgs are also isolated and purified from their natural source as well as from a heterologous expression system such as E. coli. Likewise, SAg-containing polypeptides (e.g., SAg fusion products) are isolated and purified from a heterologous expression system. In addition, Staphylococcus strains producing high levels of enterotoxin have been identified and are available. For example, exposing enterotoxin-producing Staphylococcus aureus to mutagenic agents such as N-methyl-N-nitro-N-nitrosoguanidine results in a 20 fold increase in enterotoxin production over the amounts produced by the parent wild-type Staphylococcus aureus strain (Freedman M A and Howard M B J. Bacteriol., 106:289(1971)).

6. Glycosylated SAgs and SAgs Conjugated to Glycosylceramides, Lipopolysaccharides, Glycans and Lipoarabinomannans: Presentation on CD1 Receptors for Activation of T or NKT Cells and Differentiation to Tumor Specific Effector Cells

In a tumor cell or accessory cell, nucleic acid signal sequences are integrated into nucleic acids encoding the SAg molecules in order to route them to the Golgi apparatus and endoplasmic reticulum of tumor cells where they are glycosylated via appropriate glycosyltransferases (precedents from the selective transferases used to produce monogalactosylceramide in the Sphingomonas paucimobilis) to produce a proteoglycan with structural similarity to LPS, lipoteichoic acid, GalCer, α-Gal, Streptococcus capsular polysaceharide. This construct is then secreted as an immunogenic “ground substance.” Alternatively, the resulting SAg glycolipid is anchored to the membrane, expressed on the cell surface and routed specifically to CD1 receptors.

SAgs which are glycosylated by the above intracellular processes have improved capacity to bind surface structures such as mannose receptors, ganglioside receptors and CD1 receptors. Generally, the nucleic acids encoding a SAg are modified to include a signal sequence for routing to the Golgi apparatus and a core sequence which initiates glycosylation. It is important that the Vβ TCR binding region is not blocked by the added carbohydrate modifications. For example, an N-linked glycosylation site (in the sequence Asn X Ser/Thr where X is any residue except Pro) is engineered into SAg-encoding acid sequences which do not functionally interfere with TCR binding and activation. The nucleic acid encoding these signal sequences and core binding glycosylation sites of SAgs are fused to nucleic acids encoding SAg and the fusion gene used to transfect tumor cells of a host. In addition, glycosylated forms of SAgs are expressed in a heterologous eukaryotic expression system such as yeast cells or baculovirus-infected insect cells. In gram negative bacteria (such as E. coli), nucleic acids encoding SAgs are fused to nucleic acids encoding LPS's, in gram positive bacteria (such as Staphylococcus or Streptococcus), to nucleic acids encoding capsular polysaccharides and teichoic acids and in mycobacterial species to nucleic acids encoding lipoarabinan.

The gram negative bacterium Sphingomonas paucimobilis produces the monogalactosylceramide. In this bacterium, nucleic acids encoding SAgs (containing serine) are fused to nucleic acids encoding and directing the synthesis of glycosylceramides and monogalactosylceramide in particular. The resulting galactosylceramide-SAgs are powerful T cell stimulants. The same procedure is followed in bacteria which naturally produce LPS's such as E. coli, Salmonella or Klebsiella or for bacteria which naturally produce lipoarabinomannans glycans or polysaccharides containing cell walls such as Mycobacterium and Streptococcus respectively. The SAg-polysaccharide constructs bind to CD1 receptors of antigen presenting cells. They are then capable of activating NKT cells either in vivo or ex vivo to become tumor specific effector cells in response to IL-12.

SAgs are also conjugated genetically or biochemically as in Example 5 to LPS's via a natural high affinity binding site for LPS binding protein (LPB). Once bound, the SAg catalyzes the binding of LPS monomers to CD14 and CD1 receptors in a fashion similar to that of LPB. In this way, the conjugates are capable of activating T cells for use in vivo or ex vivo for adoptive immunotherapy while preserving the anti-apoptotic effect of LPS on SAg activated T cells. Examples of their preparation and use in vivo and in vitro are given in Examples 4, 7, 15, 16, 18-23.

In addition, SAgs similarly conjugated to lipoarabinomannans and glycans are integrated into lymphomonocytic cell membranes via glycosylphosphatidylinositol anchors. These SAg-lipoarabinomannan complexes are expressed or secreted by antigen presenting cells or tumor cells. They are also bound to CD1, mannose or class II receptors in which form they are used to activate T or NKT cells. These constructs are administered in vivo or they are used ex vivo to produce tumor specific effector cell populations (T cell or NKT cells) which are employed for adoptive immunotherapy of cancer (Examples 5, 15-16, 18-23).

Mannose receptor expression is upregulated by cytokines. For example, accessory cells including DCs, and tumor cells express mannose receptors on their surfaces after GM-CSF treatment. SAgs are bound to mannose receptors by transfecting cells with nucleic acids encoding SAg which also consist of nucleic acids encoding signal sequences and glycosylation sites which, in the presence of appropriate glycosyltransferases, produce mannosylated SAgs. These preferentially bind to mannose receptors. In addition, glycosylated SAgs bind to amphipathic cell surface gangliosides and glycolipids via hydrophobic interactions. These glycosylated SAgs presented in a form bound to mannose receptors are capable of activating T cells and NKT cell populations. They are used either in vivo by direct administration or ex vivo to produce a tumor specific effector cell population (T cell or NKT cells) for use in adoptive immunotherapy of cancer (Examples 4, 5, 15, 16, 18-23).

7. SAgs Conjugated to Glycosylceramides, Gangliosides and Verotoxins (VT)

Amphipathic gangliosides bound to tumor cell surfaces such as GD1, GD2, GD3, GM1, GM2, GM3, GQ1 and GT1 are capable of binding exogenous SAgs. The binding of a SAg to the surface of a tumor cell creates an immunogen on the tumor cell surface. Tumor cells transfected with nucleic acids encoding glycosyltransferases overexpress gangliosides, producing a greater surface density of ganglioside moieties available to bind exogenous SAgs. Enterotoxins bind to cell surface amphipathic gangliosides and/or glycophorins via their hydrophobic residues while preserving their T cell binding properties. SAgs are also glycosylated intracellularly by addition of a glycosylation site or by chemical conjugation of a carbohydrate moiety using methods well described in the art. In glycosylated or native form, the SAgs bind to surface ganglioside while retaining their T cell activating properties. Overexpression of the hydrophobic regions of the molecule promotes binding to the surface gangliosides (Example 5). Examples from nature of exogenous proteins that bind to cell surface gangliosides include falciparum malarial merozoite which combines with gangliosides associated with the Duffy blood group and induce long standing and durable protection and tetanus toxin which binds to surface gangliosides with highest affinity for the disialyl groups linked to inner galactosyl residues.

Enterotoxin B contains a T cell activating sequence which is chemically cross-linked or polymerized using bifunctional agents such as carbodiimide, glutaraldehyde or formaldehyde by established methods well known in the art. These polymers are then bound to gangliosides expressed on tumor cells such as GD1, GD2, GQ1,GD3 or GM1, GM2, GM3, GT1. In monomeric or polymerized form, SAgs also bind to monogalactosylceramides which are free or bound to CD1 receptors on tumor cells or antigen presenting cells via hydrophobic interactions. The monogalactosylceramide binds to hydrophobic sequences on the SAg which are expressed at multiple sites on the molecule. In one embodiment, the lauroyl group [CH3(CH)10CO] or the group [CH3(CH))13CO] is covalently added to each of the peptide's amino terminus to serve as a of the CD1 receptor. The key SAg peptide sequence such as of SEB (amino acids 225-234) which confers T cell activating properties is tandemly repeated to various lengths prior to lipid conjugation.

Hydrophobic SAg peptides(such as Trp, Tyr, Phe, Leu, and Ile) are screened for binding to glycosylceramides immobilized on CD1 receptors or via adsorption chromatography with immobilized glycosylceramide. The SAg sequences with the greatest affinity for the CD1 receptor are selected for conjugation to the glycosylceramides and LPS's. Alternatively, the SAg sequence is screened for affinity for the CD1 or MHC class II receptor using a peptide phage display library as described in Examples 4. Likewise, preformed SAg-glycosylceramide or LPS complexes are also screened for affinity for the CD1 or MHC class II receptor (Example 4). These lipopeptide complexes are then screened for T cell proliferative activity and IL-12 production. The monomeric or polymerized SAg in native or glycosylated form binds to the monoglycosylceramides or gangliosides expressed on CD1 receptors on the tumor cell surface.

Therapeutic Construct: SAg-Glycosylceramide Conjugates

SAgs have an affinity for glycosphingolipids especially those with terminal or subterminal Gal(α1-4)Gal residues. Such residues are expressed on tumor cells as Gal(α1-4)Gal(β1-4)GlcCeramide (globotriaosylceramide or Gb3) and Gal(α1-4)GalCeramide (galabiosylceramide or Gb2). Gb3 and Gb2 also known as CD77, Burkitt's lymphoma antigen, and the human blood group p^{k} antigen are the natural receptors for Shiga toxins and VT's . Shiga toxin, a 69-kDa complex of proteins comprised of five b-subunits (7 kDa each) and one a-subunit (30 kDa) has high affinity for the terminal digalactose of Gb3 or Gb2. Methods for their preparation and isolation are described in Example 41. Once bound to the tumor cell, these toxins are internalized and induce apoptosis.

The synthetic pathway for neutral glycosphingolipids in eukaryotic cells is known. Glucosylceramide (GlcCer) is the precursor of lactosylceramide (LacCer), which leads, in order, to Gb3 and globotetraosylceramide (Gb4). Different Golgi enzymes are responsible for addition of monosaccharides from nucleotide-sugar donors in each step of the pathway. Globotriaosylceramide synthase (UDP-galactose:lactosylceramide a1-4-galactosyltransferase) has been purified. In the cytoplasm, the a-subunit of the Shiga toxin or VT is processed by a trypsin-like cleavage. The “activated” 27-kDa a-subunit inactivates 60S ribosomes by depurination of a single nucleotide in 28S rRNA, rendering ribosomes incapable of carrying out peptide elongation.

The present invention provides therapeutically active soluble complexes comprising SAg and glycosphingolipids which have terminal or subterminal Gal(α 1-4)Gal residues and Shiga toxin receptors Gb3 and Gb2, (collectively referred to as “GTSG1-4”). These complexes include but are not limited to SAg -GPI-GTSG1-4 complexes, and synthetic and functional derivatives thereof. Such structures appear naturally on surfaces of certain tumor cells such as astrocytoma, Burkitt's lymphoma and ovarian carcinoma. Methods of preparing and isolating glycosylceramides and VTs are given in Examples 41 and 55. SAgs also have a demonstrable affinity for galactosylceramides containing Gal(α 1-4)Gal residues. Methods of assessing SAg binding to GTSG1-4 are provided given in Example 43. These conjugates are also shed from SAg-transfected tumor cells as binary complexes of SAg-GTSG1-4 or ternary complexes of SAg-GPI-GTSG1-4, in free form, as vesicles or as exosomes(see Sections 38 and Example 38). Methods of isolating and characterizing these shed complexes appear in Section 38 and Example 42. The complexes may also be prepared by chemical or genetic methods (Example 5). SAg-GTSG1-4 or SAg-GPI-GTSG1-4 complexes or exosomes are useful as a preventative vaccine or against established tumor. They are also useful in vivo by direct administration or ex vivo where they are loaded onto antigen presenting cells comprising CD1 or MHC receptors to activate NKT and T cells to produce tumor specific effector T or NKT cells for adoptive therapy of cancer (Examples 5, 7, 14, 15, 16, 18-23, 38).

Therapeutic Construct: Tumor Cells Expressing SAgs and Galactosylsylceramides

Additional immunogenic complexes comprising SAgs bound to tumor cells, DCs DC/tc constructs expressing surface Gb2 and Gb3 or other glycosphingolipids containing terminal Gal(α 1-4)Gal are prepared by transfecting these cells with nucleic acids encoding a SAg. The transfected cell expresses the SAg in the context of the glycosphingolipid comprising the terminal or subterminal Gal(α 1-4))Gal moiety. Alternatively, free or GPI linked glycolipids containing SAg peptides or polypeptides bind to tumor cells or accessory cells in tissue culture (Section 38). The expression of Gb3 and Gb2 on tumor cells is optionally upregulated by various cytokines, including IFNα and TNFa, before contacting the SAg

Tumor cells, accessory cells or fused tumor/accessory cells transfected with SAg which are not naturally endowed with the GalCer (optionally coupled to SAg) acquire these molecules in free or GPI-linked form from surrounding media or by transfer from liposomes or vesicles (exosomes) which express them (Section 38 and Example 5). The resulting cells, coexpress SAgs and glycosylceramides or other glycosylceramides capable of stimulating an effective T or NKT cell immune response. Multidrug resistant (MDR) tumor cells or cell lines which naturally accumulate and express intracellular glycosylceramides are useful in this invention. MDR agonists such as SDA PSC 833, a cyclosporin analogue, and fumonisin B1, a ceramide synthase inhibitor, are employed to induce ceramide accumulation in MDR cells (Example 45). Tumor cells or accessory cells which overexpress key glycosylceramides due to transfection with aα 1-2, α1-4, α1-6 glycosyltransferases (Example 38) or a natural or induced deficiency of a-galactosidase are also useful. In addition, tumor cells with high concentrations of GalCer expressed on their surface or that of accessory cells are generated by incubation with ceramides containing a 2-hydroxy fatty acid C6OH. Tumor cells selectively convert them to GalCer, galabiosylceramide and sulfatide in the trans-Golgi network where they are sorted and transported selectively to the cell surface. Methods for this selective biosynthesis of GalCer with hydroxy fatty acids are in Example 46.

These fused SAg-tumor cell/accessory cell constructs are used to activate a T or NKT cell population. They are used in vivo by direct administration or ex vivo to produce a population of tumor specific effector cells (T cells or NKT cells) for adoptive therapy of cancer (Examples 5, 7, 14, 15, 16, 18-23, 38).

SAg-VT Conjugates to Induce Tumor Cell Apoptosis

The present invention contemplates the induction of apoptosis in tumor cells expressing Gb2 and Gb3 (or other glycosphingolipids containing terminal Gal(α 1-4)Gal) by using free SAgs, conjugates and fused DNA that comprises SAg, SAg peptide or SAg-encoding DNA fused to intact VT or to VT A or B chains. Preparation of these conjugates and fusion proteins from their corresponding DNA, polypeptides or functional derivatives is provided in Examples 1 and 5. These conjugates induce apoptosis by binding to tumor cell glycosphingolipid receptors having terminal Gal(α 1-4)Gal. Methods of assessing tumor cell apoptosis are in Example 44. CD19 or IFNα peptide sequences and generic carbohydrate recognition domains which bind Gal(α 1-4)Gal structures are also useful. CD19, a B-cell restricted differentiation antigen, naturally binds to Gb3 and Gb2 on the cell surface which incudes apoptosis. CD19 has VT-like sequences in the N-terminal extracellular domain (NBRF protein data bank) that have 41%, 34% and 37% sequence identity to VT1, VT2, and VT2e B subunits, respectively. When compared to a consensus VT B sequence, the CD19 sequences show 49% identity. Binding of these peptide sequences to membrane-Gal(α 1-4)Gal containing glycolipids facilitates receptor mediated induction of apoptosis.

The IFNα receptor has a 63-kDa extracellular peptide with regions of amino acid identity to domains in the VT B subunit implicated as Gb2/Gb3 binding sites.

The preferred targets of the above conjugates on tumor cells are the naturally expressed Shiga toxin receptors Gb3 and Gb2 with a terminal-Gal(α 1-4)Gal. Astrocytomas and Burkitt's lymphomas are the preferred tumors as they naturally express glycosphingolipid receptors. However, any tumor expressing the appropriate receptor is appropriate. Tumor cells which express either engineered or natural functional derivatives, or mutants of these glycosphingolipid receptors, are also useful. Receptor expression on the target cells is optionally upregulated by cytokines such as IFNγ and TNFα. Tumor cell sensitivity to the cytotoxic effects of a VT is enhanced by administration of interleukin-1b before the addition of the conjugates. Tumor cells which do not naturally display Gb3 or Gb2 acquire these structures by transfer from free, soluble structures or liposomes which express the missing glycosphingolipid receptor (Section 38, Example 5). The reconstituted tumor cells bearing the appropriate glycolipid receptors are thus targeted for apoptosis by the above constructs and conjugates.

SAg Nucleic Acid-Verotoxin Conjugate

A preferred construct is the SAg-VT conjugate wherein the SAg is preferably in nucleic acid form (prepared according to Example 3). The VT portion of the complex binds to the tumor cell and initiates apoptosis. The VT also acts as a “vector” for transfer of the SAg nucleic acid into the cell. SAg-VT conjugates bind to the terminal-Gal(α 1-4)Gal. receptors on tumor cell surfaces and are internalized via endocytosis. The SAg nucleic acid is internalized together with the VT. The VT A chain is an RNA N-glycosidase acting on the 60S ribosomal subunit. It induces apoptosis in the tumor cell by removing an adenine base on amino acyl-transfer RNA so that peptide chain elongation is blocked. The resulting apoptotic tumor cells contain the internalized SAg nucleic acid and are then ingested by dendritic cells. The DCs are cross primed to induce an effective anti-tumor response by presenting the tumor associated antigens in the class I pathway to T cells while the SAg nucleic acid expresses SAg polypeptide. These activated DCs or DC/tc hybrids can be prepared by the methods of Examples 28-29. They are used to activate a T or NKT cell population in vivo as a preventative vaccine or by direct administration against established tumor. They are also used ex vivo to produce a population of tumor specific effector cells (T cells or NKT cells ) for adoptive therapy of cancer (Examples 5, 7, 14, 15, 16, 18-23, 28-29).

Glycosylation or lipid binding of the enterotoxin does not interfere with T cell binding and activating properties. The SAg is glycosylated by chemical or recombinant techniques described in the Examples 4. The SAg glycoprotein is the further conjugated to gangliosides in the ganglioside synthetic pathway via the presence of key signal peptides on the glyco-SAg (Example 4).

The SAg is also rerouted to the LAMP pathway, glycosylated in the Golgi apparatus and the endoplasmic reticulum and then translocated to the membrane class II receptor as a glycosylated ganglioside. Gangliosides are glycosylated to form glycosylceramides by recombinant techniques as described in the Example 4. They are also glycosylated by glycosyltransferases to form homologues which bind to hydrophobic regions of the SAg peptide. The final products namely SAg-glycosylceramides or SAg-LPS's then bind to CD1 receptors and are used to activate T cells or NKT cells. These construct are administered directly vivo or they are useful ex vivo to produce a population of tumor specific effector T cells or NKT cells for adoptive immunotherapy of cancer by protocols given in Examples 7, 15, 16, 18-23).

The present invention contemplates the fusion or coexpression within the same cell of SAg polypeptides with anomeric mono and digalactosylceramides which are expressed within a tumor cell or on the tumor cell surface. These construct could also be effectively expressed on the surface of accessory cells defined in Oxford Dictionary of Biochemistry and Molecular Biology 1997 edition as any one of various types of cell which assist in the immune response cell and includes but is not limited to DCs, fibroblasts, synoviocytes, astrocytes antigen presenting cells, neutrophils, macrophages, basophils, eosinophils, mast cells, keratinocytes and platelets, as well as fusion cells comprising accessory cells and tumor cells.

The anomeric mono and digalactosylceramides have been shown to activate NKT cells and to produce an anti-tumor response in the context of IL-12. The galactosyl ceramides have several structural requirements in order to produce anti-tumor effects. 12. Mono and digalactosylceramides require an anomeric galactose or glucose as the terminal sugar or inner sugar as for example anomeric 1,6-digalactosylceramide, -anomeric 1,2-digalactosylceramide, anomeric 1,4-digalactosylceramide, a diglycosylceramide wherein the inner sugar is an anomeric galactose or an anomeric glucose and anomeric galactosyl or anomeric glucosyl ceramide. In addition, the 3- and 4-hydoxyl groups on the phytosphingosine portion of the ceramides are preferably unsubstituted, the sphingosine base length is preferably from about 10 to about 13 carbon units and the fatty acyl chain length is preferably in the range of about 12 to about 24 for optimal anti-tumor effectiveness of the molecule.

The expression of anomeric mono- and digalactosylceramides in a cell is achieved by several methods. The first involves the transfection and amplification of nucleic acid encoding the enzymes which synthesize the anomeric 1,4-, the anomeric 1,6- or the anomeric 1,2.- mono- and digalactosylceramides such that these glycolipids are overproduced. The genes for these transferase enzymes have been cloned. Transfection of nucleic acid encoding these terminal transferases into the above cells is carried out in vivo by the methods described in Example 1.

A second method for creating cells that overexpress the foregoing glycolipids uses monensin or brefeldin which block additional glycosylation and sialylation of the -galactosylceramides, so that the mono- and digalactosylceramides accumulate in the cell.

A third approach employs cells from patients with Fabry's disease. These cells are genetically deficient in the -galactosidase so they naturally accumulate -galactosylceramides.

In a fourth technique, an -galactosidase deficiency is induced in the target cell so that -galactosylceramides accumulate.

In a fifth approach, the -galactosyltransferase is transfected into Fabry's disease cells, thereby adding to the usual accumulation due to the catabolic enzyme deficiency. Such cells should have massive accumulations of -galactosylceramides.

In a sixth approach, the desired mono- or diglycosylceramide expressed on liposome surfaces are transferred to tumor cells lacking these structures by co-culture and employment of fusion techniques given in example 5.

Nucleic acids encoding SAgs are transfected into the above cells which are overexpressing, overproducing or otherwise accumulating mono and digalactosylceramides. The Golgi apparatus (or Golgi complex) is a major site of synthesis of the foregoing glycolipids. In the present context, the SAg combines with it the mono and digalactosylceramides. From the Golgi, the SAg-galactosylceramide conjugates or complexes, with the appropriate sorting signals, are dispatched in transport vesicles to other destinations. For a SAg peptide to combine effectively with an -galactosylceramide, the peptide must first have the appropriate sorting signal which directs it to the Golgi and from there, after complexing with the glycolipid, to the cell surface. The trafficking pathway of SAg polypeptide from the ER to the Golgi does not require special signals. SAg polypeptides that enter the ER (and fold and assembles properly) will automatically be transported through the Golgi apparatus to the cell surface unless they carry signals that either detain them in an earlier compartment en route or divert them (via the Golgi apparatus) to lysosomes or secretory vesicles. The SAg-glucosylceramide conjugates are routed from the Golgi to the cell surface after acquiring a structure like a cytoplasmic tail such as phosphoinositol which assures that these molecules will be bound in the cell membrane. The conjugates may also be routed to CD1 or MHC class I receptors, or via, the class II pathway, to MHC class II receptors by associating with invariant chain or LAMP-1 signals as described in Section 8.

The mono- and digalactosylceramides are capable of stimulating NKT cells (via an invariant chain) in the presence of IL-12 to produce an anti-tumor response. SAgs are capable of stimulating a T cell-dependent anti-tumor response.

The present invention utilizes tumor cells, accessory cells or hybrid cells such as DC/tc, engineered to express SAg-galactosylceramide for anti-tumor therapy. These cells may be administered as a preventative or therapeutic vaccine (Example 29). Alternatively, they may be useful ex vivo to activate an NKT or T cell population for use in adoptive immunotherapy of cancer (Example 29).

8. SAg Targeting to Lysosomes

LAMP-1 is a transmembrane protein localized predominantly to lysosomes and late endosomes. The cytoplasmic domain of LAMP-1 contains the amino acid sequence (SEQ ID NO: 29) Tyr-Gln-Thr-Ile whose structure conforms to the Tyr-Xaa-Xaa hydrophobic amino acid motif that mediates cell membrane internalization and possibly lysosomal targeting of several surface receptors. The intracellular targeting of LAMP-1 is controlled by the (SEQ ID NO: 29) Tyr-Gln-Thr-Ile motif located at the C terminus of its cytoplasmic tail.

In the present invention, nucleic acid encoding a SAg is fused with nucleic acids encoding the transmembrane and cytoplasmic tail of LAMP-1. Nucleic acids encoding the signal peptide (N terminal) of LAMP-1 are integrated into this chimeric construct. These chimeric SAg/LAMP-1 polypeptides are targeted to endosomal and lysosomal compartments, thereby rerouting transfected SAg polypeptides into the MHC class II processing pathway. Thus, cells such as tumor cells transfected with nucleic acid encoding this modified SAg preferentially target the SAg to lysosomal compartments and are presented to T cells in the context of MHC class II. MHC class II negative tumor cells are also transfected with nucleic acid encoding MHC class II molecules. The association of SAgs with MHC class II molecules, their natural ligands on APCs, produce optimal T cell activation to the tumor. Antigen presenting cells transfected with these constructs are capable of inducing potent activation of T cells. Tumor cells, in particular, transfected with this construct are administered directly in vivo or used ex vivo to sensitize a T cell population which is useful in adoptive immunotherapy of cancer by protocols described in Example 16, 18-23).

10. SAg Receptors

It is clear that certain tissues express receptors for enterotoxins that are not MHC class II and that binding is reserved for selected enterotoxins and not others. Non MHC cell II binding has been reported for colon carcinoma, mast cells epithelial cells and B cells. In a tumor bearing patient, it is desirable for administered SAgs to target tumor cells in vivo. which naturally express enterotoxin binding sites or receptors. Natural ligands for these receptors are native enterotoxins. However, because of the existence of naturally occurring enterotoxin specific antibodies in the circulation, native enterotoxins are incapable of binding target tumor cell or T cells. The isolated receptor is used to screen and identify SAg proteins and/or nucleic acids which bind to the native or chimeric receptor. SAg constructs are produced which target the tumor via its SAg receptor while also retaining T cell activating properties. In addition, T cells or NKT cells from tumor bearing patients are anergized in the course of tumor growth and are incapable of being used as a source of T cells for ex vivo stimulation and adoptive immunotherapy. After transfecting these cells with nucleic acids encoding enterotoxin receptors, they are capable of responding to exogenous enterotoxins and are once again a source of T cells useful in adoptive immunotherapy of cancer by protocols given in Examples 8, 9, 12, 16, 18-23.

Methods for receptor isolation purification and retrieval of cDNA are given in Example 12. The nucleic acids encoding SAg receptors are transfected into cells by methods given in Example 1 Tumor cells have a natural binding site for exogenously administered SAg polypeptides. In addition, nucleic acid encoding the SAg receptors are transfected into T cells, NKT cells, or γ/δ T cells of cancer patients which have been anergized in the course of tumor growth. The expression of the SAg receptor permits these cells to proliferate and produce TH1 cytokines in response to exogenous native SAg, Hence, these autologous T cell populations are useful in adoptive immunotherapy. Likewise, accessory cells are transfected with SAg receptor genes and used ex vivo to present SAg to T cells. Further, the nucleic acid encoding the SAg receptor is transfected into T cells and fused, in frame, to the nucleic acid encoding the TCR-associated zeta chain or the IL-2 g to produce a chimeric receptor capable of generating a signal for cell proliferation and the release of TH1 cytokines after binding its natural ligand exogenous SAg.

In one embodiment, the enterotoxin receptor is immobilized as in Example 12 and used to screen oligonucleotide libraries for binding (Gold L, J. Biol. Chem. 270:13581-13584 (1995)). Avidly binding oligonucleotides are used to mimic the native enterotoxin by targeting the receptor in vivo. They are coupled to the TCR binding site of an enterotoxin peptide. In this way, the hybrid molecule is administered to the patient in a form protected from circulating enterotoxin-specific antibodies. Additionally, a nucleic acid molecule is prepared which mimics the enterotoxin in its ability to bind to the enterotoxin receptor on tumor cells and to the TCR on T cells. This nucleic acid mimicking the native enterotoxin is administered to the tumor bearing patients and is capable of targeting the enterotoxin receptor sites on tumor cell and the TCR without being eliminated by circulating enterotoxin specific antibodies as in Example 13, 18, 20-23.

11. Tumor Cells that Express SAgs and the αGal Epitope

Tumor cells are for the large part weakly antigenic and poorly recognized by the immune system. various attempts to increase the immunogenicity of tumor cells by transfection of various cytokines or histocompatibility antigens have for the most part been unsuccessful. Hyperacute rejection of xenografted organs is a very rapid and dramatic immune event often occurring within minutes of vascularization of the xenografted organs. Very recently, a major antigenic system on xenografts which is the target of this reaction has been identified as αGalβ1-3Galb β1-4GlcNAc or αGal. This epitope is expressed in the tissues of pigs, guinea pigs, rodents, dogs, and cows but has not been detected in human tissue. The present invention improves the antigenicity of tumor cells and their recognition by the immune system by providing the Gal epitope on the cell surface either alone or together with SAg expression.

The αGal epitope is expressed by endothelial cells in xenografts such as pig organs is a major antigenic target causing hyperacute organ rejection in human transplant patients. This hyperacute rejection appears to involve a complement dependent mechanism that occurs within a few minutes. An α1-3-galactosyltransferase is an enzyme capable of producing α 1-3-galactose-β1-4-N-acetylglucosamine moiety by adding a terminal galactose residue to a subterminal galactose residue via an a1-3 linkage. In addition, the α 1-3-galactosyltransferase is not expressed by human and certain primate cells. Humans contain xenoreactive natural antibodies that recognize αGal. For example, anti-Gal antibodies bind to pig endothelial cells that express the Gal epitope. These anti-Gal antibodies are naturally occurring IgM antibodies recently found to be present in large amounts in human serum. Surface expression of the αGal epitope on tumor cells is achieved by transfecting a cell with a cDNA clone encoding the α1-3-galactosyltransferase. While tumor cells are the preferred cells for transfection, other cells such as accessory cells or immunocytes are also contemplated as being within the scope of this invention.

Nucleic acids encoding α1-3-galactosyltransferase polypeptides are known (Sandrin, M S et al., Proc. Natl. Acd. Sci. USA 90: 11391-11395 (1993)). A cDNA clone encoding murine 1-3-galactosyltransferase is prepared using the known sequence of this protein and the polymerase chain reaction (PCR) technique (Dabrowski, P L et al., Transplant. Proc. 26: 1335-1337 (1994). Briefly, two oligonucleotide primers are synthesized: 5′-GAATTCAAGCTTATGATCACTATGCTTCAAG-3′, which is a sense primer that encodes the first 6 amino acids of the mature α1-3-galactosyltransferase and contains an HindIII restriction site; and 5′-GAATTCCTGCAGTCAGACATTATTCTAAC-3′, which is an anti-sense primer that encodes the last 5 amino acids of the premature 1-3-galactosyltransferase and contains an in-frame termination codon and PstI restriction site. These primers amplify a 1185 bp fragment from a C57BL/6 spleen cell cDNA library that is subsequently purified, digested with HindIII and PstI (Pharmacia LKB) restriction endonucleases, and directionally cloned into HindIII/Pst I-digested expression vector such as CDM8 vector. After verifying the correct sequence, the α1-3-galactosyltransferase-containing expression vector is transfected into heterologous cells such as COS cells to confirm activity. Activity can be confirmed by testing transfected cells for Gal expression using the IB4 lectin (Sigma) of Griffonia simplicifolia that binds to αGal residues.

In the preferred mode, cells transfected with nucleic acids encoding a SAg are co-transfected with nucleic acids that encode an -galactosyltransferase. Alternatively, nucleic acids encoding the transferase are transfected into a separate cell population which is coadministered with the SAg transfected cell population.

The SAg-encoding nucleic acid can be transfected into cells which already express Gal epitope. In addition, any cell can be transfected with the -galactosyltransferase-encoding nucleic acid. For example, αGal-negative human tumor cells or tumor cell lines such as melanoma or adenocarcinoma are transfected with nucleic acid encoding the -galactosyltransferase. Tumor cells transfected with -galactosyltransferase-encoding nucleic acid express the αGal on their surface and are rapidly rejected when administered to a host with preexisting αGal specific antibodies. Methods of transfection are given in Example 1.

Human tumor cells expressing the αGal epitope after transfection, become strongly reactive with human serum containing preexisting antibodies to the αGal epitope. Thus, an αGal-expressing tumor cell is rejected after implantation.

The ability of αGal-transfected tumor cells to induce rejection is demonstrated by implantation into severely compromised immune deficient (SCID) mice that have been reconstituted with human T and B cells and transfused with normal human plasma containing the naturally occurring human antibodies specific for the αGal epitope. In this case, tumor cells transfected with -galactosyltransferase-encoding nucleic acid is rejected while untransfected cells are not. Similarly, tumor cells transfected with -galactosyltransferase-encoding nucleic acid is rejected when implanted into species such as humans which synthesize antibodies to the αGal epitope compared to untransfected control tumor cells that are unaffected by the treatment.

For example, pretreatment with 10⁵-10⁷-galactosyltransferase transfected tumor cells subcutaneously followed by implantation of untransfected tumor cells prevents the outgrowth of untransfected malignant tumor cells. Hence, the -galactosyltransferase transfected tumor cells function as a vaccine. Further, -galactosyltransferase transfected cells implanted into animals after untransfected tumors are established induce rejection of an established untransfected tumor.

To test for the presence of αGal on a cell surface, α1-3 galactosyltransferase knockout mice that do not express the Gal antigen are used. The α1-3 galactosyltransferase knockout mice are described elsewhere (Tearle et al., Transplantation 61:13-19 (1996) and Shinkel et al., Transplantation 64:197-204 (1997)). A syngeneic tumor cell that is αGal negative such as B16 melanoma variants is transfected with nucleic acids that encode a given carbohydrate modifying enzyme. These transfected cells are then implanted into the knockout mouse that received plasma containing αGal specific antibodies. Tumors do not grow in animals containing Gal specific antibodies if the Gal epitope is expressed. Thus, hosts implanted with Gal positive tumor cells exhibit less growth than those exhibited in hosts implanted with tumor cells that are Gal negative.

αGal negative transgenic animals are prepared which are useful for testing αGal expressing tumors. To produce these animals, nucleic acids encoding αGal fucosyltransferase are transfected into αGal positive mice. The fucosyltransferase dominates the usage of substrate N-acetyllactosamine and precludes -galactosyltransferase from utilizing this substrate. The transgenic mice do not express—αGal on the cell surface. In this way, transgenic mice with the H antigen rather than the αGal antigen develop. Transgenic guinea pigs producing minimal αGal are also created in this way. These animals are used as models for testing their capacity to reject syngeneic αGal positive tumors. These systems also permit the testing of αGal specific antibodies for anti-tumor effects after they are passively infused into animals bearing αGal positive tumors.

Neuroblastoma and some melanoma cells overexpress several disialogangliosides, for example, GD2 and GD3. In the present invention, nucleic acid-encoding specific sialidases or glucosidases or neuraminidases that cleave terminal sialic acid or carbohydrate residues are transfected into cells that then express or overexpress a ganglioside with an exposed αGal epitope.

Fucosylated glycolipids such as B group antigens, Lewis blood group antigens, and L-selectin ligands are converted to the αGal epitope using the appropriate sialidases and glycosyltransferase enzymes. For example, a desialylating enzyme is introduced into B group antigen expressing cells such that the—1,3-linked galactose is exposed and now recognized by αGal antibodies. Mild acid treatment to remove the branching fucose residues on the fucosylated B antigen is used to expose the a1,3 galactose residues.

Alternatively, cells expressing the B antigen or selectin antigen are transfected with -galactosyltransferase-encoding nucleic acid that competes successfully with fucosyltransferases for N-acetyl-lactosamine substrate and preferentially expresses the αGal epitope

Nucleic acid encoding other polypeptides are also used to produce the surface expression of the Gal epitope such as nucleic acid encoding glycosidases that specifically cleave carbohydrate residues to expose the αGal epitope. Tumor cells transfected with nucleic acids encoding N-acetyl-glucosaminyl transferase show an increased tendency to metastasize and colonize new organs. These same tumor cells are cotransfected with nucleic acids encoding SAgs, Staphylococcal hyaluronidase and erythrogenic toxins as well as Streptococcal capsular polysaccharide which enables them to secrete enzymes and toxins locally inducing a potent inflammatory and immune response at metastatic sites.

Co-transfection of tumor cells with nucleic acid encoding SAg and nucleic acid encoding a galactosyltransferase, sialidase, and/or glycosyltransferase results in expression of SAg, GalCer, αGal, or other glycolipids on the cell surface. These tumor cells are used to stimulate T or NKT cells ex vivo to produce a population of tumor specific effector cells which are deployed for adoptive immunotherapy of cancer.

Mutation of the glycosyltransferase nucleic acid in tumor cells produces a specific LPS containing the Gal/Cer or the αGal which coordinated with genes for protein glycosylation produce the desired integrated SAg LPS.

13. Tumor Cells Expressing SAgs Glycosylceramides and LPS's and Their Receptors

It appears that anti-tumor responses are produced by a subpopulation of T cells known as NKT cells. These cells recognize glycosylceramides with certain specifications which are presented in the context of CD1 receptors on antigen presenting cells. They produce IL-12 mediated anti-tumor responses. Peptides of certain length with hydrophobic sequences have been shown to react with various hydrophobic regions of the CD1 receptor and produce an immune response. However, these peptides have not been implicated in an anti-tumor response. In the present invention, lipoproteins are contemplated which consist of SAg or their major bioreactive domains fused to glycosylceramides in the context of the CD1 receptor.

To make this construct, CD1 positive cells are transfected with nucleic acids encoding glycosyltransferases that result in GalCer or GlcCer expression on the cell surface and preferably in the context of the CD1 receptor. The appropriate glycosyltransferase nucleic acid is obtained from Sphingomonas paucimobilis or Agelas mauritianus which are known to express the GalCer on their cell surface. The GalCer and GlcCer moieties are recognized by NKT cell Va invariant chains in the context of CD1 receptors on antigen presenting cells. CD1 positive cells are cotransfected with nucleic acids encoding SAgs The resulting CD1 positive cells coexpress both GalCer and SAg on the cell surface or in the context of CD1. The GalCer and SAg presented simultaneously as a complex and/or separate from each other on the cell surface, in the context of CD1 produces potent activation of NKT cells due to recognition of SAg by NKT cell Vb chain and GalCer by the Va invariant chain. Such GalCer-SAg complexes are loaded onto the CD1 receptor and presented to NKT cells in this fashion. A SAg peptide capable of binding to the TCR and activating the T cell is useful for coupling to the Gal-Cer before or after it is positioned on the CD1 receptor. (See Examples 1-4, 5). CD1 positive antigen presenting cells or tumor cells bearing the SAg glycosylceramide are used to stimulate a population of NKT cells ex vivo which is then useful in adoptive immunotherapy of cancer by protocols given in Examples 7,15, 16, 18-23). They are also useful when administered directly in vivo to tumor bearing patients to produce an anti-tumor response. (See Examples 18-23).

In the present invention, nucleic acids encoding the CD1 receptor are transfected into tumor cells in vivo or ex vivo. Martin L H. et al. Proc. Natl. Acad. Sci USA 83: 9154-9158 (1986). Nucleic acids encoding the CD1 receptor are also cotransfected into tumor cells with nucleic acids encoding the SAg receptor. A tumor cell expressing a chimeric receptor comprising sequences of CD1 and SAg receptors is also produced by transfection of fusion nucleic acids encoding both receptors. The transfected tumor cell expresses either dual or chimeric receptors which bind SAg and GalCer independently or as fusion protein or conjugate. Likewise, tumor cells are transfected with nucleic acids encoding CD14, the LPS receptor, (Ferrero, E. et al., J. Immunol. 145: 331-336 (1990)) a leucine rich receptor glycoprotein found on myeloid cells with a LPS binding site between amino acids 57-64. Nucleic acids encoding CD14 are transfected into tumor cells together with nucleic acids encoding SAgs and resulting tumor cell expresses several receptors or a single chimeric receptor with preserved consensus binding sequences common to each. These tumor cell transfectants are capable of binding exogenous SAg and/or LPS and or GalCer. The resulting tumor cells with bound SAg, and/or Gal/Cer and/or LPS activate a population of T cells and/or NKT cell to produce tumor specific effector cell which are useful in the adoptive immunotherapy of cancer by methods in Example 1-7, 12, 15, 16, 18-23). The tumor cell transfectants are also administered as a vaccine or to hosts with established tumors as in Example 19-23.

Alternative splicing and utilization of cryptic splice sites generates alternative reading frames and secretory isoforms of CD1, CD14 and SAg receptors. Woolfson A. et al., Proc. Natl. Acad. Sci. USA 91: 6683-6687 (1994). These soluble receptors are immobilized on solid surfaces such as polystyrene plates or beads and bind their respective ligands e.g. GalCer and SAg. In this form, the GalCer and SAgs activate T cell or NKT cell to produce a population of tumor specific effector T cell or NKT cells useful in adoptive immunotherapy of cancer by methods given in Examples 7, 15, 16, 18-23).

14. SAg-Activated Tumor Specific T Cells, NKT Cells or γ/δ T Cells Expressing CD44 for Adoptive Immunotherapy

It is imperative that T cells, NKT which are stimulated in vivo or ex vivo by the SAg constructs given herein are capable of trafficking and homing effectively to tumor sites. CD44 expression on T cells after SAg stimulation, is an indicator of upregulated adhesive capacity which is requisite for the homing of SAgs to tumor sites. T cells or NKT cells or cells transfected with nucleic acids encoding SAg receptors i.e. tumor cells or accessory cells are stimulated by SAgs in vivo or ex vivo to express CD44. These CD44 expressing T cells are enriched and expanded and then harvested for use in adoptive therapy of cancer by protocols given in Examples 7, 15, 16, 18-23).

Transfection of cDNAs encoding soluble isoforms of CD44 into tumor cells results in the local release of soluble CD44 which inhibits the ability of endogenous cell surface CD44 to bind and internalize hyaluronate and to mediate tumor cell invasion. Mice injected with tumor cell transfected with the CD44 isoform showed not tumor metastases. Such tumor cells were shown to undergo apoptosis. These transfectants displayed a marked reduction in their ability to internalize and degrade hyaluronate. Therefore, CD44 function promotes tumor cell survival in invaded tissues possible as a result of impairing their ability to penetrate the host tissue hyaluronan barrier. In the present invention, SAg-encoding nucleic acid is co-transfected or fused to nucleic acids encoding CD44 isoforms. These transfected cells are capable of migrating to sites of metastatic tumor in tumor bearing hosts and eliciting a potent anti-tumor response. The combined apoptotic effect to the CD44 isoform with the enhanced immunogenicity of the SAg produces a powerful synergistic anti-tumor response. The nucleic acids encoding the CD44 isoform and SAg are transfected into accessory (DC)/tumor cell hybrids. In addition, to presenting tumor antigen and SAg to the immune system and inhibiting metastases, the CD44 isoform produces apoptosis of the fusion cell which in turn is ingested by DCs resulting in enhanced immunogenicity and a more potent tumoricidal response. These combined transfectants are used preferably against established tumor according to protocols in Example 19-23.

NKT cells or T cells that do not produce CD44 after SAg stimulation do so after transfection with nucleic acids encoding CD44 or transferases such as N-acetylglucosaminyl transferase III or CD44 (Sheng, Y. et al., Int. J. Cancer 73, 850-858 (1997); Nottenberg, C. et al., Proc. Natl. Acad. Sci. USA 86: 8521-8525 (1992)). The latter enzyme synthesizes bisecting N-acetylglucosamine structures on asparagine linked * oligosaccharides. Glycosylation of CD44 by these transferases produces enhanced CD44 mediated adhesion to immobilized hyaluronate. SAgs are used to activate T cells which have been transfected with nucleic acids encoding N-acetylglucosaminyltransferase III. The SAg stimulated transfectants display increased CD44-mediated adhesion. as well as lymphocyte homing and trafficking. Certain T cell, NKT cell or /T cell populations which are unable to express CD44 after SAg stimulation are transfected with nucleic acids encoding CD44 before sensitization with SAgs. These cells express CD44 after immunization with SAgs in vivo or in vitro. These additional populations of effector T cells are useful in adoptive immunotherapy of cancer by methods given in Examples 5, 7, 15, 16, 18-23.

15.Tumor Associated Antigens Include

(1) Normal structures, e.g., differentiation or tissue specific antigens,

(2) Mutated normal structures

(3) Products of alternate reading frame or fusion of several genes

(4) Chimeric products resulting from cell or gene fusion

(5) Xenogeneic antigens (“xenoantigens”)

A tumor antigen (also called “tumor associated antigen) is any antigenic structure expressed by a tumor cell. For example, tumor antigens include mutated products of various oncogenes and p53 genes that are expressed in tumor cells generally. Many tumor antigens associated with particular types of cancers are known. For example, tumor antigens associated with breast, colon, and lung cancer are known and have been cloned. Common melanoma antigens recognized by T lymphocytes have been identified and are used as immunotherapeutic antigens for treatment of melanoma. Five genes encoding different melanoma antigens have been identified. For example, MAGE1 and 3, expressed on melanoma and other tumor cells, are recognized by cytotoxic T lymphocytes (CTL) in the context of HLA-A1 (Van der Bruggen P et al., Science 254:1643 (1991) and Gauler B et al., J. Exp. Med. 179:921 (1994)). MART-1 identical to Melan-A (Kawakami et al., Proc. Natl. Acad Sci. USA 91:3515 (1994) and Coulie et al., J. Exp. Med. 180:35 (1994)); gp100 (Kawakami et al., Proc. Natl. Acad. Sci. USA 91:6458 (1994)); and tyrosinase (Brichard et al., J. Exp. Med. 178:48 (1993)) are melanocyte lineage-related antigens expressed on both melanoma and melanocytes. MART-1 and gp100 have been shown to be recognized by MHC-class I-restricted CTL in the context of HLA-A2, and tyrosinase in the context of HLA-A2 and HLA-A24 [Robbins et al., Cancer Res. 54:3126 (1995)]. An additional list of tumor antigens useful in this invention is given in Rosenberg, S A. Principles and Applications of Biologic Therapy in Cancer: Principles and Practice of Oncology DeVita, V T., Hellman, S., Rosenberg, S. A., eds, J. B. Lippincott Co. Philadelphia, Pa. 1993.

In addition tumor associated antigens are defined as including normal structures expressed in tumor cells, mutated normal structures, normal differentiation- or tissue-specific structures, products of alternate reading frames of the same genetic regions, chimeric products of several genes that originated in a parental or in a fused, hybrid cell. This also includes gene products expressed in association with MHC molecules or other surface receptors, organelles or vesicles.

Tumor cells expressing tumor-associated antigens are transfected in vivo or ex vivo with nucleic acids encoding a SAg alone or together with nucleic acids encoding other products, such as those listed in Tables I and II. These include surface antigens and receptors such as the α Gal epitope, GalCer, CD1, CD14 and SAg receptor. The transfected cells may be of host origin, or syngeneic, allogeneic or xenogeneic; the cells may be non-malignant. SAg-encoding nucleic acid may also be inserted into a mutated normal gene in a tumor cell, e.g., LDL receptor gene in a melanoma cell. The LDL receptor is expressed as a fusion product of the LDL receptor gene (chromosome 19) and a fructose transferase gene on the same chromosome. This combination results from chromosome inversion which gives rise to the fusion product probably due to recombination between the two ends of this chromosome. The expressed peptide epitope is therefore a nonsense sequence being read in the wrong direction. The three base pair mutations in the third open reading frame results in the expression of a mutant peptide. Site directed mutagenesis can be achieved by insertion of SAg-encoding nucleic acid into the mutant gene at any feasible site or by targeting insertion in place of the mutated base pairs. The resultant LDL receptor displays the SAg alone or as a chimera with the mutant sequence. Site directed mutagenesis by SAg-encoding nucleic acid may also target the b-catenin gene which in melanoma shows a single C-T mutation which results in a ser to phe substitution and the generation of the 9 amino acid mutant peptide. The SAg-encoding acid may be inserted or may substitute for any sequence in a normal non-mutated gene, a tissue-specific or differentiation-associated gene in tumor cells, or other genes expressing their products in a tumor cell. Preferably, the mutated gene product is immunogenic and recognized as a dominant epitope by the host immune system, preferably by T cells (including tumor infiltrating lymphocytes). The mutated sequence may, in contrast, be a weak immunogen which is rendered more immunogenic when presented in the context of a SAg.

In the preferred embodiment, the transfected cells are tumor cells of host origin expressing a defined tumor associated antigen such as MART-1. If the tumor antigen is not expressed or weakly expressed on the transfected cells, then the tumor cell is transfected with nucleic acids encoding an immunogenic tumor antigen such as MART-1, tyrosinase or MAGE-1 in addition to SAg and other constructs described herein.

The tumor cells may be transfected in vivo by administering nucleic acids encoding SAgs and/or the other nucleic acid constructs described above using a site directed mutagenesis approach in vivo and methods such as described in Example 1, 3, 18-23. Tumor cells may also be transfected ex vivo by methods given in Example 1-3. Ex vivo transfected tumor cells are used as vaccine or to treat established tumor by methods and protocols in Example 18-23 They are also useful ex vivo to immunize T cells or NKT cells to produce a population of tumor specific effector cells adoptive immunotherapy of cancer by methods and protocols given in Examples 7, 15, 16, 18-23.

16.Immunostimulatory Sequences

Several of constructs consist of nucleic acids encoding SAg peptides which produce anti-tumor responses by activating host TH-1 CD4+ T cells to proliferate and produce tumoricidal cytokines such as IL-1α, IL-1bβ, IL-2, IL-6, TNFα, TNFβ and IFNγ. The incorporation of the immunostimulatory sequence into the genetic construct of SAg DNA, ensures that the T cell response is skewed to produces a predominant proliferation of TH1 cells and production of a TH1 cytokine profile. Immunostimulatory sequences (ISS) consist of DNA sequences that exhibit immunogenicity. Briefly, plasmid DNA (pDNA) having short immunostimulatory DNA sequences containing a CpG dinucleotide in a particular base context were shown to be immunogenic (Tokunaga J et al., J. Natl. Cancer Inst. 72:955-962 (1984)). By synthesizing single stranded nucleotides corresponding to different regions in the Mycobacterium bovis genome, specific single stranded oligonucleotides that activate adherent splenocytes and enhanced natural killer cell activity have been identified. In addition, single stranded oligonucleotides with CpG motifs induce B cell proliferation and secretion of IL-6 and IFN (Krieg et al., Nature, 374:546 (1995)). The activation capability generally has the formula 5′-Pur-Pur-C-G-Pyr-Pyr-3′. Further, human monocytes transfected with pDNA or double stranded oligonucleotides containing ISS transcribed large amounts of IFNγ and IL-12 (Sato et al., Science 273:352-354 (1996); Zhu et al., Science 261, 209-211, (1993)) Direct gene transfer with plasmid-cationic liposome complexes resulted in lasting, generalized or tissue specific expression of the injected genetic phenotype.

In the present invention, the ISS is inserted into nucleic acid sequences of SAgs and tumor associated antigens which are used to transfect tumor cells, antigen presenting cells, accessory cells including muscle cells in vitro or in vivo by methods given in Example 1-3, 15, 16, 18-23. In all instances, the SAg stimulation of the T cell response is critical to an effective anti-tumor response of the host. The presence of the ISS ensures that the SAg nucleic acids preferentially activate the TH1 after in vivo administration of the nucleic acids encoding SAg. SAg DNA is useful ex vivo in activating T cells by direct transfection or by presentation via incubation with pretransfected antigen-presenting cells or tumor cells. The tumor specific T effector cell are then useful for adoptive therapy of cancer using protocols given in Examples 7, 15, 16, 18-23). A particularly useful method involves the intratumoral injection of nucleic acids encoding SAgs. The latter is administered in naked, plasmid or liposomal form. Once tumor inflammation is initiated (generally within 15 days after injection), the host is given T cells or NKT cells which have been immunized in vitro to the tumor by tumor cells transfected with nucleic acids encoding SAg plus additional constructs given in Tables 1 and II by methods given in Examples 7, 15 16 18-23.

17. Liposomes

Liposomes containing repeating units of the Gal epitope, GalCer, and/or SAgs are constructed and administered directly into a tumor. These elements are combined before incorporation into liposomes or they are added individually in the preparative procedure. Methods for preparation of these liposomes are given in Examples 5. These liposomes are preferentially delivered parenterally or directly into the tumor. The administration of SAgs in this manner provides a high local concentration of SAg to stimulate an anti-tumor response. These liposomes are also useful ex vivo by activating a T cell or NK T cell population which is then harvested and used for adoptive immunotherapy as described in protocols in Examples 5, 7, 15-17, 18-23).

18. Tumor Cells that Induce Cellulitis

Transfection with microbial nucleic acids that encode tissue spreading factor (hyaluronidase), erythrogenic toxins, enterotoxins; capsular polysaccharides from S. aureus and Streptococcus pyogenes, S. aureus and S. pyogenes have potent tissue invasive properties. Specifically, Staphylococcus and Streptococcus are capable of invading tissues by secreting several enzymes which lyse ground substance such as mucopolysaccharide, hyaluronic acid, or chondroitin sulfate, create local thrombosis, and initiate inflammation and edema. These enzymes consist of hyaluronidase, streptokinase, streptodornase, erythrogenic toxins as well as various enterotoxins (Example 3). In the present invention, the nucleic acid sequences encoding these potent enzymes are transfected into tumor cells, either in vitro or in vivo (Examples1-3, 6, 15, 16, 18-23). In vivo, the transfected tumor cells migrate to sites of existing metastases. The transfected tumor cells secrete the enzymes which hydrolyze the tumor ground substance and neovasculature and toxins to induce inflammation and an immune response in tumor tissue. Tumors which are encased in nests of connective tissue are eliminated by this process. The resulting increase in local vascular permeability induced by the combined effect of enzymes and toxins produces intense inflammation at tumor sites. If their administration is timed to the peak of tumor inflammation, liposomes as described herein and chemotherapy are sequestered and concentrated in the inflamed tumor bed producing an augmentation of the tumoricidal response.

A relatively low number of transfected tumor cells with the complete microbial enzymatic and toxin genetic construct would be required to induce a tumoricidal effect. The population of transfectants would then proceed to secrete these microbial enzymes locally. In addition, nucleic acid encoding these enzymes are derived from a strain of Staphylococcus or Streptococcus such as Staphylococcus epidermidis or of low or intermediate virulence.

Tumor cells are cotransfected with glycosyltransferases or treated with glycosyltransferase-inducing agents resulting in the expression of the Gal epitope and reduction in the survival time of tumor cells For example, the nucleic acids encoding the glycosyltransferase from Sphingomonas paucimobilis or Agelas mauritianus produce GalCer are transfected into tumor cells to induce the surface expression of GalCer or Gal. The tumor cells then express and/or secrete microbial agents such as SAgs, hyaluronidase and crythrogenic toxins that hydrolyze the ground substance of the tumor. By also displaying SAgs and α-Gal or Gal/Cer epitopes which activate NKT cells, T cells, and Gal specific antibodies the transfected tumor cells induce profound tumoricidal activity. These transfected tumor cells are used to activate a population of T cells to become tumor specific effector cells which are employed for the adoptive immunotherapy of cancer. See Examples 1, 2, 4-5, 7, 15, 16, 18-23.

For in vivo transfection of tumor cells, the microbial genetic nucleic acids are targeted to tumor cells as described herein (See p. 12 “Transfection”, Examples 1-3, 6, 19). Once localized in tumor sites in vivo, the tumor cell is capable of hydrolyzing surrounding stroma and, initiating thrombosis, inflammation, and increased tissue permeability. Additional microbial nucleic acid encoding proteinases, lysoproteinases, tissue spreading factors, a and b hemolysins and toxins are also transfected into tumor cells and used in accordance with this invention.

Micrometastatic disease in cancer patients is of great concern as it often goes undetected and is refractory to chemotherapeutic agents. Documented metastases in breast cancer patients is associated with a poor prognosis. The present invention contemplates that the metastatic properties of tumor cells coupled with the potent inflammatory properties of the microbial products are useful in tracking and eliminating micrometastatic disease in tumor bearing patients. Tumor cells are transfected with nucleic acid encoding polypeptides involved in metastasis. These include but are not limited to peptides that upregulate the adhesive properties of CD44 (e.g., glycosyltransferases), the c-erbB-1 encoded EGF receptor which is associated with enhanced metastases in breast carcinomas or c-erbB-2/neu encoding the p185 receptor associated with poor prognosis in breast and ovarian carcinomas. These cells with metastatic activity are programmed to traffic, home and colonize specific sites of existing metastases the tumor bearing host. Hence they have the unique property of charting the micrometastatic sites of the tumor. These tumor cells are cotransfected with microbial nucleic acids encoding the hyaluronidase, erythrogenic toxin and enterotoxins as well as the αGal. Hence, as they colonize metastatic sites, these transfectants induce a potent inflammatory and immune response. This ensures their own destruction together with the surrounding untransfected micrometastatic tumor cell population and neovasculature and stroma. Methods of preparation, administration and assessment of these transfectants in tumor bearing hosts are in Example 1-3, 18-23.

The tumor cells are also transfected with the above microbial genes on a DNA template with a tissue specific promoter in order to target the activity of these transfected tumor cells to the vital organs (and sites therein) affected by the existing metastatic tumor. For breast cancer, this would be lung, liver or brain. These organ-specific promoters ensure that the expression of the microbial products would occur in the organ(s) targeted by the tissue specific promoter

The same tumor cells are also provided with inducible promoter sequences which control the level of receptor transcription and expression. Inducible promoters suitable for use in mammalian cells include the MMTV-LTR under the control of steroid hormones and the metallothionein promoter under the control of heavy metal ions. In this case, the microbial nucleic acids are linked to steroid inducible gene sequences. Transcription is triggered when these cells are exposed to a threshold level of steroids. Hence, two to three days after administration to the host, when the above transfectants have colonized tumor metastatic sites, a bolus of corticosteroid is administered which initiates transcription of the microbial enzymes and toxins by the tumor cell transfectants and their secretion. In this fashion, the transfected tumor cells express and secrete their inflammatory products in metastatic tumor sites resulting in the elimination of metastatic disease.

19.Tumor Cells as Mimics of Virulent Bacteria: Transfection with Nucleic Acid Encoding Bacterial Invasins, Virulence Factors, and Enzymes that Degrade Extracellular Matrix

Tumor cells with a metastatic phenotype are transfected with nucleic acids encoding proteins with the capacity to invade and adhere to inflammatory cells such as macrophages (adhesins and virulence factors). These genes are inducible and controlled by operons.

SAg-encoding nucleic acid is fused in frame to nucleic acid encoding oncogenes involved in tumorigenesis and metastasis. Examples of such genes, in addition to erb/neu, erb, erbB2 and EGF (epidermal growth factor receptor) discussed above, include ras and mutated ras, erk, and mtal, 182mts1, nm23 (See Table 9.5, p181 of Franks L. M. et al., Cellular and Molecular Biology of Cancer, Oxford University Press, Oxford UK, (1997) which is incorporated by reference), as well as the laminin-integrin and the cadherin family. These genes are particularly useful because they are overexpressed in tumor cells displaying a metastatic phenotype.

Invasins

SAg-encoding nucleic acid is fused in frame or cotransfected into tumor cells with nucleic acids encoding bacterial invasins and hyaluronidases. The invasin imparts leukocyte like activity to bacteria is transfected into tumor cells which allows the tumor cells to penetrate tissues. These are exemplified by Yersinia pseudotuberculosis invasin and hyaluronidase (including its various isotypes) and also known as tissue spreading factors. The invasin gene exemplified in Y. pseudotuberculosis encodes a protein located in the outer membrane of the bacterium called invasin (Inv) and the gene is known as inv. The DNA region of the inv gene contains a open reading frame 2964 bases. This protein binds to the host cell surface by means of the C-terminal 192 residue region. Mutation by insertion of a transposon or elimination of the inv gene greatly impairs the ability of the bacterium to penetrate tissues (Schaecter M et al., Genetics of Bacteria edited by Baer G M et al., in Mechanisms of Microbial Disease Williams and Wilkins Baltimore (1993)).

The host membrane receptors for invasin belong to the integrin superfamily with a particular affinity for VLA-3, 4, 5, 6. Invasin also bind to T cell a₄β₁ which is involved in lymphocyte homing or traffic. Once bound to a phagocyte, phagocytosis is triggered and the bacterium is taken up. Nucleic acids encoding Inv are transfected into tumor cells which confers upon the tumor cell a phagocytosis triggering signal for host macrophages.

E. coli genes of the P pili or pap operon encoding adhesin proteins have been isolated from chromosomes and plasmids. The gene cluster is linked to genes for other virulence determinants such as the KI capsular polysaccharide and hemolysin. The receptor for the pili is the αGal(1-4)Gal moiety of the P blood group antigen. Examples of host cell receptors for bacterial adhesins is given in Table 7.2 of Patrick and Larkin. Pilin genes in N. meningitidis encode proteins is which the fimbriae are the N-methylphenylalanine pili. An extensive region of amino acid homology at the N-terminal end is common to a wide range of bacterial genera including Pseudomonas aeruginosa, N. gonorrhoeae, N. menigitidis, Moraxella bovis and Bacteroides nodosus. This N-terminal region is highly hydrophobic which is in contrast to the fimbriae of the Enterobacteriaceae which either have a hydrophobic region at the C-terminal end or lack a hydrophobic region altogether. Of interest is the presence of a site on SAgs which resembles the third Ig-like disulfide-bridged loop of VCAM-1 and a conserved sequence is present within the same subregion of the fifth Ig-like VCAM-1 loop. The only known receptor for the VCAM-1 is VLA-4, an adhesion molecule expressed primarily by activated T and B cells. A survey of target cell susceptibility to SEC dependent lysis shows a correlation between VLA-4 expression and susceptibility to lysis.

Hyaluronidases and Proteases

Bacteroides species produce hyaluronidase, heparanase, and chondroitin sulfatase enzymes. C. perfringens m toxin is a hyaluronidase enzyme and Bacteroides and C. perfringens produce elastase and collagenase enzyme while Porphyromonas gingivalis has a cell associated collagenase. Streptococcus pyogenes produces hyaluronidase enzymes which depolymerize their own capsules. Neuraminidases and endoglycosidases, lipases, nucleases and proteases produced by a wide variety of bacteria are also useful in this invention as capable of promoting tissue necrosis in tumor masses and/or tumor nests.

The staphylococcal invasive genome is predominantly chromosomal and the nucleic acid segments encoding the major invasive enzyme systems, permeability factors, and toxins have been isolated, cloned, and sequenced. For example, the nucleic acid sequence encoding a hyaluronidase from group A Streptococcus strain 10403 is described elsewhere (Hynes et al., Infect. Immun. 63:3015-3020 (1995)). Tumor cells transfected with nucleic acids encoding microbial invasive and inflammatory substances are preferentially used in vivo where they are programmed to traffic to metastatic sites and/or organs primarily infiltrated by the tumor. Once situated in tumor, they commence secretion of their inflammatory enzymes and toxins. Protocols for their preparation, use, and assessment are given in Examples 1-3, 18-23.

Consolidation of Bacterial Genes

The microbial nucleic acids encoding hyaluronidase, erythrogenic toxins proteases, coagulases and enterotoxins are consolidated into a chimeric construct or plasmid and transfected into tumor cells which then commence secretion of the spreading factors, pro-inflammatory and permeability inducing agents. For example, a single construct or multiple constructs contains the nucleic acid encoding polypeptides including, without limitation, enterotoxin B, hyaluronidase, streptokinase, coagulase, Staphylococcal protease and erythrogenic toxins.

Tumor cells transfected with the above microbial genes are prepared as in Example 1-3 and are used in the treatment of established and metastatic tumor or as a preventative vaccine as described in Examples 15-23.

20. Combined Expression of Different Stimulatory Molecules by Co-Transfection of Tumor Cells or Fusion of Singly Transfected Cells

Tumor cells that express two different types of exogenous molecules are produced by either cotransfection of the same cells with (a) SAg-encoding nucleic acid and (b) nucleic acid encoding a toxins or autolysin, or by fusion of tumor cells that have been singly transfected with (a) with tumor cells transfected by (b)

Tumor cells are provided which have the dual capacity to colonize metastatic tumor sites in vivo and induce inflammation. Once situated in sites of tumor metastasis, the tumor cells behave like a necrotizing bacterium or leukocyte. For example, tumor cell are transfected with nucleic acids encoding bacterial invasins to promote adhesion, “tissue spreading factor” or hyaluronidase to hydrolyze the ground substance, coagulase to induce local thrombosis and streptokinase and streptodornase. In addition, tumor cell are provided with nucleic acids encoding bacterial toxins which bind and produce autolysis and cytotoxicity for surrounding tissue and tumor cells. The tumor cells are also cotransfected with additional nucleic acids encoding SAgs. The toxin genes useful herein are amplified by providing two copies tandemly duplicated on a chromosome and linked to an amplified oncogene. Situated in tumor tissue, these transfected tumor cells release enterotoxins as well as inflammatory enzymes, immunogenic capsular lipoproteins, cell wall LPS's and cytolysins. This evokes a potent T cell and inflammatory response in tumor tissue. These inflammatory genes are inducible at the level of the operon or in some instances bacteriophage which controls their activation. Transfected tumor cells are transfected with microbial nucleic acids given above either in vitro or in vivo at tumor sites as in Example 1-3, 5, 16-23 and p.11 under “transfection”.

The S. aureus a toxin forms pores or transmembrane channels in a wide range of host cells. It is released from the bacteria during exponential growth and has a molecular mass of 33 kDa. Expression of the gene encoding the a toxin, hly, is under the control of the agr gene which coordinately controls the expression of a number of extracellular proteins, including exfoliatin toxin, toxic shock syndrome toxin, a, b, and d toxins, enterotoxin B, lipases and nucleases. The b toxin is a phospholipase which attacks a sphingomyelin in the cell membranes. The phage encoding the toxin is hlb. Exfoliatin toxin A is encoded by a chromosomally located gene eta and the gene for toxin B is etb. The eta gene is by the agr gene regulator which is a member of the histidine-protein kinase response regulator superfamily. (Patrick S et al., Immunological and Molecular Aspects of Bacterial Virulence, John Wiley and Sons New York, N.Y. 1995)

SEB binds to glycosphingolipids on cell membranes. The ganglioside binding site on SEB is overexpressed, or a myristoylation site or GPI binding site is integrated into its structure so that it is bound to the surface of the tumor cell membrane and not secreted. The SEB will preferentially bind to tumor cell expressing ganglioside tumor associated antigens and will augment the immunogenicity of these antigens. S. aureus produces a bifunctional protein autolysin of 110-kDa,(HlyA) via the atl gene that has an N-acetylmuramoyl-L-alanine amidase domain and an endo-b-N-acetylglucosaminidase domain. It undergoes proteolytic processing to generate two extracellular enzymes that are secreted. The specific secretion proteins HlyB and HlyD are 80 kDa and 54 kDa respectively. The process is directed by the hlyB and hlyD genes which are contiguous and co-expressed with the hylC and hylA genes that are required for the synthesis of protoxin and the acyl carrier protein-dependent fatty acylation that matures it to cytolytically active toxin. Hemolysin is secreted as the mature acylated form of the hlyA gene product proHlyA following the covalent attachment of a fatty acid moiety in a cytoplasmic mechanism directed by the dimeric HlyC activator, a putative acyl transferase and dependent upon the acyl carrier protein. This specific and novel HlyC-directed fatty acylation is required to target the hemolysin toxin to mammalian cell membranes prior to forming cation-selective pores and disrupting the host cell.

Bacteria such as E. coli, Bordetella pertussis, Pasteurella haemolytica, Proteus vulgaris and P. mirabilis produce genetically related toxins. Their activity is dependent on the presence of calcium ions. Characteristically, they have regions of 10 to 47 repeats within the amino acid sequence and termed repeats in toxin or RTX gene family. The repeat sequence contains the following nine amino acids; (SEQ ID NO: 34) leucine-X-glycine-glycine-X-glycine-asparagine-aspartic acid-X where X is a variable amino acid. These repeats are required for hemolytic activity. A large hydrophobic region of the hemolysin separate from the repeats, is also essential for activity and may be involved in the a interaction with the host cell membrane. The hemolysin A of E. coli apparently form pores on the target cell membrane. This requires a 20 kDa product of another gene HlyC before it becomes actively hemolytic. In E. coli, the operon for the production of the hemolysin contains four genes hlyA which codes for the structural hemolysin and hlyC which is required for activation of the HlyA . The other two genes hlyB and hlyD are involved in the transport of HlyA to the extracellular environment. Pasteurella haemolytica leukotoxin and Bordetella pertussis adenylate cyclase hemolysin have similar C-terminal sequence and associated genes analogous to those in the hly operon. (Koronakis V et al., Secretion of Hemolysin and other Proteins out of the Gram-Negative Bacterial Cell, in Ghuysen J M et al., ed, Bacterial Cell Wall, Elsevier, Amsterdam (1994)).

The Shiga toxin of Shigella dysenteriae and Shiga-like toxins of E. coli (Verotoxins) are a family of related toxins which have similar amino acid sequences and biological activities. The A subunit of Shiga toxin has a molecular mass of 31 kDa which associates with five to the 7 kDa B subunits. The A subunits is proteolytically cleaved into A1 and A2. It is the A1 fragment which is biologically active. The host cell receptor for Shiga toxin is the glycolipid Gal(α1-4)Gal(β1-4) GlcCeramide (globotriosylceramide; Gb3) and for Shiga-like toxin I (SLTI) and SLTII of E. coli is Gal(α1-3)GalCeramide (Galabiosylceramide). The binding specificity is dependent on both sugars residues and the lipid moiety. The Shiga toxin is known to inhibit protein synthesis. It is a RNA N-glycosidase enzyme whose site of action is the 60S ribosomal subunit. The toxins remove an adenine base from position 4324 on the aminoacyl-transfer RNA binding site of 28S ribosomal RNA hence preventing peptide length elongation. The effect on protein synthesis is similar to that of diphtheria toxin and Pseudomonas aeruginosa exotoxin A. The SLTI and II toxins of E. coli and encoded by lysogenic phage. Its expression is controlled by iron concentration in the growth medium by way of the fur gene and iron box repressor protein binding site. Clostridia difficile toxins A and B also bind to anomeric galactose epitopes on cell membranes and induce membrane associated enzymes and inhibit G protein activation which results in cell death. Tumor cells transfected with a galactosyltransferase genes to produce the α-Gal epitope are a susceptible to lysis by both the Shiga-like toxins and C. difficile toxin. The expression of the α-Gal epitope is enabled by the transfection of nucleic acids encoding α-Gal-transferase into tumor cells.

Listeria monocytogenes produces a hemolysin. listerolysin O (LLO), a member of the thiol-activated family of cytolysins. LLO is encoded by the gene hyl (also designated hylA and lisA). Listerolysin O toxin is a pore forming toxin which degrades the membrane of its phagocytic vacuole allowing the bacterium to escape into the host cytoplasm. This gene cloned into Bacillus subtilis enables the bacterium to grow rapidly intracellularly in the cytoplasm of a macrophage-like cell line after disrupting the phagosomal cell membrane.

Tumor cells are transfected with the above microbial nucleic acids as in Example 1-3. These transfectants are useful in vivo against established tumor and micrometastatic disease (Examples 5, 15, 16, 18-23).

21. Augmentation of Tumor Cell Immunogenicity by Bacterial Products: Transfection with Genes Encoding Bacterial Antigens or Receptors for Bacterial Products

Tumor cell are provided with augmented antigenicity by expressing fundamental patterns that are recognized by fundamental recognition units of the innate immune response. Examples are LPS's of gram negative organisms, SAgs and peptidoglycans of gram positive organisms, fungal β-glucans, bacterial glycosylceramides, and mycobacterial lipoarabinans. Numerous infectious agents with these structures cause potent immune reactions e.g. streptococcal cellulitis induced by S. pyogenes, E. coli induced sepsis and meningococcal meningitis induced by Neisseria meningitidis (SEQ ID NOS: 35-36).

The T cell system is far more adept at responding to innate pattern recognition units than to tumor associated antigens. In the present invention, tumor cells are transfected with nucleic acids encoding molecules or biosynthetic enzymes that result in structures which mimic the major immunogenic structures of bacterial antigens. This enables the tumor cells to be recognized more effectively by the T cell system. In addition, tumor cells are provided with receptors for bacterial antigens such as SAgs, LPS's (CD14), and glycosylceramides (CD1). Genes encoding bacterial antigens which produce potent immune responses are transfected into tumor cells to include bacterial membrane and cell wall constituents such as LPS's, peptidoglycans, glycosylceramides, lipoproteins, lipoarabinans and capsular polysaccharides. In addition, nucleic acids encoding the staphylococcal SAgs induce potent T cell lymphoproliferation and TH-1 cytokine production while LPS's are known to have a bystander effect on T cell proliferation The two agents synergize in their capacity to induce lethal endotoxic shock in animals.

The present invention contemplates that the optimal approach is to present the bacterial immunogen structure (for example streptococcal capsular polysaccharide) sequentially or concomitantly with a bacterial mitogenic signal (SAg). Under certain conditions, these genes are co-transfected with various bacterial invasins, toxins, autolysins and inflammatory enzymes which together with the colonizing properties of tumor metastasis genes produce a tumor cell capable of migrating to metastatic sites where it induces necrotizing cellulitis. Such genes are preferably placed under the control of inducible promoters as described herein.

These transfectants are prepared by methods in Example 1-3. They are useful against established tumors or metastatic tumor in vivo as in Example 15, 16, 18-23.

21b. Combining Expression of SAg Nucleic Acids with Nucleic Acids Encoding Enzymes that Drive the Synthesis of Bacterial LPS, Galactosylceramide or Capsular

Polysaccharide

In general, this is accomplished by co-transfection of nucleic acids each encoding one of the above products or by transfection with a fusion nucleic acid that encodes the combination.

SAg-encoding nucleic acid is fused in frame or cotransfected into tumor cells or accessory cells with nucleic acids encoding bacterial LPS's, peptidoglycans, and galactosylceramides. The preferred end products are synthesized in E. coli and N. meningitides (LPS's), Staphylococcus and Streptococcus (peptidoglycans); Sphingomonas paucimobilis (glycosylceramides).

The synthetic genome or cluster of genes for biosynthesis of these products is incorporated as a whole to include multiple and specific enzymatic transferases and trafficking proteins required for the stepwise synthesis of each of these products. Gene clusters are necessary to provide the requisite transferases for synthesis of these large molecules. For example the genes required for the biosynthesis of type 1 capsular polysaccharide of S. aureus are localized to a 14.6-kb region. Sequencing analysis of the 14.6-kb fragment revealed 13 open reading frames (ORFs). Ten genes are involved in capsule biosynthesis. CapG aligned well with consensus sequence of a family of acetyltransferases from various prokaryotic organisms suggesting that CapG may be an acetyltransferase.

The structural requirements for endotoxic activity of LPS's are as follows. (1) a b(1-6)-linked D-glucosamine disaccharide backbone; (2) biphosphorylation at positions 1 and 4′ of the disaccharide backbone; (3) a suitable number of 3-acyloxyacyl groups per disaccharide unit; and (4) acyl groups of a suitable length as indicated by Kumazawa et al., and Nakatsuka et al. Transfection with nucleic acid encoding LPS's would require the preservation of the biphosphorylation and the acyl groups between 14 and 23 to maintain optimal activity. Derivatives may contain a monosaccharide group in place of the disaccharide group.

LPS Structure

LPS consists of an outer region which is composed of polymerized di- and pentasaccharide repeating units whose compositions vary within a species or strain. The inner region is generally conserved within a single genus, and consists of a core oligosaccharide linked by the sugar 2-keto-3-deoxy-D-amino-octonate (KDO to a disaccharide backbone with attached long chain fatty acids, the lipid A. This component is responsible for much of the biological activity of the molecule. Components conferring the greatest biological and immunomodulatory activity are now known to be a glucosamine disaccharide, a bis phosphorylated lipid A and acyloxyacyl groups on the fatty acid chain. The loss of only one of these components, for example, a phosphate group reduces the activity of the molecule. LPS's from different genera of bacteria vary in their immunomodulating activity and studies of the structure have shown very subtle differences. For example, Bacteroides spp. is apparently less active in endotoxin activity than LPS from enteric bacteria. This was initially thought to be related to a modification of the of KDO in the core region with an added phosphate group. Other differences in the LPS were found when the fatty acids from E. coli and Bacteroides were compared. E. coli has six fatty acid chains or acyl groups per diglucosamine backbone each with a chain length of 12-14 carbon atoms. Included in the acyl groups is 3-hydroxytetradecanoic acid (3-OH-C14:0)which is absent in the Bacteroides strains. In contrast, Bacteroides has 4-5 fatty acids of chain length 15-17 carbons per diglucosamine and has branched 3-hydroxy fatty acids. Studies of synthetic lipids have confirmed that reduced biological activity relates to fewer fatty acids chains.

A common feature of LPS's from various species is that they are amphiphiles, with both a hydrophobic part capable of dissolving in lipid membranes and a hydrophilic part which remains in the water phase. Therefore, the first step of molecular interaction is one between the amphiphilic molecule and the mammalian cell surface either by ionic binding, hydrogen bonding or hydrophobic interaction. The bacterial molecule may be inserted into the mammalian membrane by its hydrophobic moiety or attached to membrane receptors with the hydrophilic moiety, or through charge effects or via binding to host glycoproteins and glycolipids resulting in signal transduction. Most of the immunomodulating activity of these bacterial molecules is indirect and stems from the release of host mediators. Cytokines such as IL-1, tumor necrosis factor, and IL-6 are involved. The LPS binding protein attaches to gram-negative bacteria or free LPS and mediates the attachment to macrophage membrane receptor known as CD14. The recognition of the CD14 only recognizes LPB when it is bound to LPS. The LPS-LPB complex may directly trigger TNF release or hold the complex at the cell surface so that other hosts cell surface molecules trigger TNF release. LPBs also act as opsonins. Another area where sugar residues play an important role is in cell surface glycoprotein interactions which involve protein-carbohydrate recognition. In the recirculation of and recruitment of leukocytes in the body, the carbohydrate-recognizing protein domains of glycoproteins of one cells bind specifically to the oligosaccharides of glycoconjugates on another type of cell. These recognition events control the movement of bloodborne lymphocytes into lymphoid organs. Specific recognition occurs between lymphocytes and specialized cells in the wall of blood vessels known as high endothelial venules.

Genes Encoding Lipid A Biosynthesis

LPS is generally synthesized as two separate components, the lipid A/core and the O polysaccharide, which are then ligated to give the complete LPS molecule. Three genes encode enzymes that catalyze the steps of lipid A synthesis (lpxA, lpxD and lpx B for steps 1,3 and 5) and fabZ and envA. More specifically, the enzymes that catalyze the synthesis of lipid A are thought to act in the following sequence (indicating the genes): lpx A, lpx C, lpx D, lpxB. The reactions catalyzed by the products of these genes are a given in Table 1 of Schnaitman C A et al., Microbiol. Rev. 57: 655-682 (1993).

Blocks of Genes Involved in LPS Biosynthesis

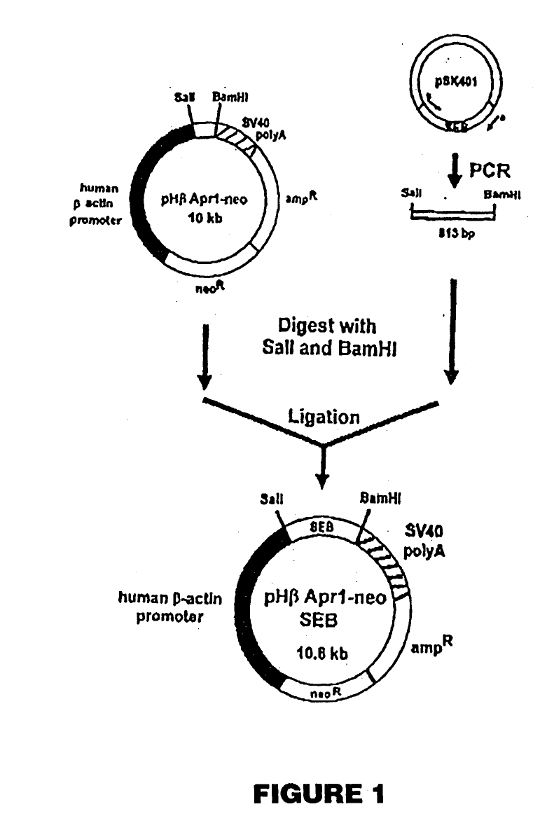

Blocks of genes involving LPS synthesis have been sequenced and analyzed. The lipid A biosynthetic pathway has been elucidated. Four of the genes in this pathway have now been identified. Three of them are located in a complete operon which also contains genes involved in. DNA and phospholipid synthesis. Genes involved in synthesis of the LPS lipid A core are given in Tables 1 and 2 and their activity at various points in the biosynthetic pathway are given in FIG. 1 of Schnaitman C A et al., Microbiological Reviews 57: 655-682 (1993). which is incorporated by reference. Therefore, it is likely that LPS biosynthetic enzymes are organized into clusters on the inner surface of the cytoplasmic membrane around a few key membrane proteins.

A cluster of assembly genes produced by various bacteria encode LPS with homologous structures. These genes have been transfected into E. coli and they induce identifiable LPS's. There are also smooth and rough LPS's which have a hierarchy of potency in terms of procoagulant activity and activation of TNF. Mutants produced which synthesized progressively less polysaccharide attached to the lipid A moiety. The presence of long chain polysaccharides attached to the lipid moiety decreased the ability to activate TNF. Rough bacteria were more effective than smooth bacteria in inducing TNF production. Fatty acids of various chain lengths can be produced including those that resemble monogalactosylceramides. Transferases for biosynthesis of galactan the LPS structure of the O antigen from Klebsiella pneumoniae have been identified as well as genes controlling the O antigen chain length.

The genes for LPS and glycosylceramide assembly also involve multiple transferases. The transfection of tumor cells involves 10 genes encoding a particular stretch of the bacterial genome. In E. coli, the 14-kilobase pair chromosomal region located between waaC (formerly rfaC) and waaA (kdtA) contains genes encoding enzymes required for the synthesis and of the type R2 core oligosaccharide in the lumen of the endoplasmic reticulum. This occurs in a stepwise fashion. The gene encoding the Haemophilus influenzae type B outer membrane protein functions as a porin and is useful in protective immunity has been cloned as a 10-kilobase Hib DNA insert and expressed in E. coli. The biosynthesis of LPS's involves genes encoding the key transferases including rfaI. The N. meningitides highly conserved surface protein conferring protection is encoded by a ORF of 525 nucleotides.

Genes Encoding Enzymes the Catalyze Core Biosynthesis

The rfa cluster includes the genes for all transferases for assembly of core. It includes three operons consists of at least 17 genes. The majority of known genes whose functions are involved exclusively in LPS core biosynthesis are located in the rfa cluster [Pradel E et al., J. Bacteriology 174: 4736-4745 (1992)]. It includes three operons. However, there are also genes such as kdsA and rfaE located outside the rfa cluster which are involved in biosynthesis of sugars unique to the core or exert direct effect on core structure. These clusters appear to have originated by the exchange of blocks of genes among ancestral organisms. There are few which code for the integral membrane proteins. The promoter for the rfa genes has been identified. Mutations have been identified known a rough mutants traced to three loci namely rfa, rfb and rfc.

The region of the E. coli chromosome encoding enzymes responsible for the synthesis of the LPS core has been cloned. This region formerly known as the rfa locus comprises 18 kb of DNA between the markers tdh and rpmBG. The genes are arranged in three different operons and the genetic organization of this locus seems to be identical in E. coli K-12 and S typhimurium.

Linkage of LPS Transcription and Toxin Secretion

In E. coli and Salmonella, a link has been found between toxin secretion and the gene regulating LPS transcription. Toxin secretion is regulated by gene expression within the hlyCABD operon. A recently identified activator of hlyCABD gene expression is the 128-kDa product of the rfaH (sfrB) gene which positively regulates transcript initiation and possibly termination in the operons encoding synthesis of LPS of E. coli and Salmonella. The discovery of a role in hlyCABD expression for the LPS (rfa) operon transcriptional activator rfaH is consistent with the role of LPS in influencing both the secretion and toxic activity of the toxin.

Genes Encoding Enzymes that Synthesize Polysaccharide Capsule and Membrane Proteins

Genes for the biosynthesis of a polysaccharide capsule are induced in Sphingomonas by overlapping DNA segments which span about 50 kbp restored the synthesis of sphingan. The polysaccharide components of LPS from B. Pertussis, H. influenzae and Bacteroides spp. will activate B-cells. The polysaccharide of Bacteroides activates B cells indirectly by first triggering the macrophage whereas the lipid A moiety triggers the B cells directly. Therefore different parts of the same molecule interact with different types of host cells. There is also evidence that immunopotentiating activity of a glycopeptide produced by mycobacteria is dependent on the saccharide residues of the molecule. The capsular polysaccharide of the Streptococcus is extremely immunogenic, consisting of glycan strands composed of regularly alternating N-acetylglucosamine and N-acetylmuramic acid residues joined through β1-1,4 glycosidic linkages and attached to crosslinked peptides by amide bonds. The capsule of strain M is composed of taurine-2-acetamido-2-deoxyfucose and 2acetamido-2-deoxy-D-galacturonic acid. The gene for this structure called cap-1 has been cloned and is used to transfect tumor cells. The nucleic acid sequences appear in Lin et al., J. Bacteriol. 176, 7005-7016 (1994).

A new 24-kDa group A streptococcal membrane protein known as streptococcal protective antigen (Spa) has been identified and is distinct from the surface M protein which evokes protective opsonizing antibodies. The Spa-encoding gene has been cloned and consists of a 636-bp 5′ fragment. (Dale, J B et al., J. Clin. Invest. 103: 1261-67 (1999)).

The present invention contemplates the use for cancer treatment of these and other bacterial antigens from staphylococci streptococci, E. coli, N. mengitides, and other genera which antigens evoke an immune response in mammals. In the preferred approach, a nucleic acid encoding such an antigenic structure is transfected and expressed in tumor cells. Methods of preparation, use and assessment of these therapeutic constructs in tumor bearing hosts are in Example 1, 2, 18-23.

SAg nucleic acids are fused in frame or cotransfected into tumor cells or accessory cells with nucleic acids encoding key transferases (gene clusters) and glycosylation sites encoding capsular membrane from Streptococcus or Neisseria menigitidis lipoprotein-LPS-phospholipid and cell wall peptidoglycans, i.e., N-acetylglucosamine (NAG) and N-acetylmuramic acid (NAM).

SAg DNA is fused in frame to DNA encoding a highly conserved outer membrane surface protein of N. meningitides known as Nspa. The Nspa gene has been cloned (Martin, D. et al., J. Exp. Med. 185: 1173-1183 (1997)). The LPS produced would be of weak to intermediate strength such as that produced by Listeria or Legionella.

Borrelia burgdorfi is the causative agent of Lyme disease. The osp genes are located at a single genetic locus on a 49 kb double-stranded DNA linear plasmid where they are organized as an operon ospAB. The amino acid sequences of OspA and OspB show a high degree of similarity and resemble prokaryotic lipoproteins. Nucleic acids encoding the ospA and ospB lipoproteins are cotransfected into tumor cells together with SAgs.

Genes Encoding Membrane Glycosylceramide Biosynthesis

Nucleic acids encoding the synthesis of the GalCer from Sphingomonas paucimobilis are transfected into tumor cells, resulting in the synthesis of GalCer by the tumor cell. (Kawahara K et al., FEBS Letters 292: 107-110, (1991) Yamazaki M et al., J. Bacteriology 178: 2676-2687 (1996) Natori T et al., Tetrahedron Letters 34: 5591-5592 (1993) Costantino V et al., Liebigs Ann. Chem. 96: 1471-1475 (1995)). Nucleic acids encoding enzymes responsible for synthesis of Neiserria meningitides LPS are transfected into tumor cells, resulting in the synthesis of LPS by the tumor cell (Steeghs L et al., Gene 190: 263-270 (1997)). These nucleic acids encoding key transferases are fused to nucleic acids encoding amplified oncogenes or transcription factors such as Bcl-2, c-myc, K ras, bcr, c-abl or NF-kB.

Genes Involved in Mycobacterial Cell Wall Biosynthesis

SAg-encoding nucleic acid is fused in frame or cotransfected into tumor cell with nucleic acids encoding the key enzymes involved in the biosynthesis of mycobacterial cell wall mycolic acid, phosphatidylinositol mannosides and lipoarabinans. A high affinity interaction of CD1b molecules with the acyl side chains of known T cell antigens such as lipoarabinomannan, phosphatidylinositol mannoside and monomycolate has been demonstrated. Hence the nucleic acid encoding the CD1 receptor are cotransfected into tumor cells together with SAg-encoding nucleic acid and nucleic acids encoding the multifunctional fatty acid and mycocerosic acid synthases involved in the biosynthesis of mycolic acid and methyl-branched fatty acids.

The multifunctional genes for mycocerosic acid synthase involved in the biosynthesis of these molecules have been isolated. In addition to the usual fatty acids found in membrane lipids, mycobacteria have a wide variety of very long-chain saturated (C18-C32) and monounsaturated (up to C26) n-fatty acids. The occurrence of a-alkyl b-hydroxy very long chain fatty acids i e., mycolic acid is a hallmark of mycobacteria and related species. Mycobacterial mycolic acids are the largest (C70-C90) with the largest-branch (C20-C25). The main chain contains one or two double bonds, cyclopropane rings, epoxy groups, methoxy groups, keto groups or methyl branches. Such acid are the major components of the cell wall, occurring mostly esterified in clusters of four on the terminal hexa-arabinofuranosyl units of the major cell wall polysaccharides called arabinogalactans. They are also found esterified to the 6 and 6′ positions of trehalose to form “cord factor”. Small amounts of mycolate are also found esterified to glycerol or sugars such as trehalose, glucose and fructose depending on the sugars present in the culture medium. Mycobacterium also contains several methyl-branched fatty acids. These include 10-methyl C18 fatty acid (tuberculostearic acid found esterified in phosphatidyl inositide mannosides), 2,4-dimethyl C14 acid and mono-, di- and trimethyl-branched C14 to C25 fatty acids found in trehalose-containing lipo-oligosaccharides, trimethyl unsaturated C27 acid (phthienoic acid), tetra-methyl-branched C28-C32 fatty acids (mycocerosic acids) and shorter homologues found in phenolic glycolipids and phthiocerol esters and multiple-methyl-branched phthilceranic acids such as heptamethyl-branched C37 acid and oxygenated multiple methyl-branched acids such as 17-hydroxy-2,4,6,8,10,12,14,16,-octamethyl C40 acid found in sulfolipids.

Genes Involved in Mycolic Acid Biosynthesis

The biosynthesis of mycolic acids involves fatty acid chain elongation, desaturation, cyclopropanation of the olefin and a Claissen-type condensation. The genes involved in cyclopropanation are cma1, cma2. The methoxymycolate series found in M. tuberculosis contains a methoxy group adjacent to the methyl branch, in addition to the cyclopropane in the proximal position. A series of four methyl transferase genes was cloned. The mm4 methylates the distal olefin.

The multifunctional fatty acid synthase (FAS) (type 1) catalyses not only the synthesis of C16 and C18 fatty acids but also elongation to produces C24 and C25 fatty acids. Cloning and sequencing of the synthase gene revealed a 8389 bp ORF. The domain organization is much like a head to tail fusion of the two yeast FAS subunits; acyl transferase (AT)-enoyl reductase (ER)-dehydratase (DH)-malonyl/palmitoyl transferase-acyl carrier protein (ACP) fused with b-ketoreductase (KR)-b-ketoacyl synthase (KS).

The MAS gene encoding mycobacterial mycocerosic acid synthase is a dimer of the FAS gene. The cloning and sequencing of the MAS gene revealed the domain organization in the following order: KS-AT-DH-ER-KR-ACP. The purified MAS shows a preference for elongation by four methylmalonyl CoA units reflecting the natural composition of mycocerosic acids. FAS and MAS are also involved in the biosynthesis of phthiocerol and phenolphthiocerol which involve elongation of preformed n-C20 fatty acyl chains or an acyl chain containing a phenol residue at the w-end. The cluster of five genes, ppsil-5 encode the multifunctional enzymes (Fernandes N D et al., Gene 170: 95-99 (1996) Mathur M et al., J. Biol. Chem. 267:19388-19395 (1992) Yuan Y. et al., Proc. Natl Acad. Sci. USA 92: 6630-6634 (1995)).

Tumor cells are cotransfected with SAg-encoding DNA and nucleic acids encoding the biosynthesis the above microbial products. The transfected tumor cells acquire significant additional immunogenicity. These cells are prepared as in Example 1-3. They are useful in vivo as a preventative or therapeutic antitumor vaccine (Examples 5 15, 16 18-23. They are also useful ex vivo to immunize T or NKT cells to produce a population of effector T or NKT cells for adoptive immunotherapy of cancer (Examples 2-5. 15, 16. 18-23).

22. SAg-Ganglioside or SAg-Galactosylceramide Complexes Formed After Transfection of Tumor Cells with DNA Encoding SAgs: Complete Bacterial Antigen System

Recognized by CD1 Receptors Capable of Inducing Anti-Tumor Effects

SAg-encoding nucleic acid transfected into tumor cells express SAg on the tumor cell surface which is bound to cell surface gangliosides which are tumor associated antigens, oncogene product such as EGF or IGF. In this way the tumor associated antigen is capable of recognition and interaction with host T cells and macrophages and of evoking a potent immune response. The SAg is also bound or associated with the CD1 receptor alone or associated with the glycosphingolipid tumor associated antigen.

SAgs have a natural affinity for glycosphingolipids on cell membranes. Enterotoxin-producing-bacteria secrete enterotoxins which in their precursor state are bound to cell membranes in dimeric form. Enterotoxin transfected tumor cells induce an anti-tumor response by expressing the tumor cell surface antigen in association with the SAg. Bound to the tumor cell membrane, the SAg may be in dimeric form associated with the ceramide lipophilic anchor domain of a glycosphingolipid tumor associated antigen. Likewise, the SAg may be associated with the carbohydrate moiety or the ganglioside which protrudes from the cell surface. It may also be secreted in monomeric or dimeric form fused to membrane associated tumor antigen, oncogene product or receptor. If the tumor associated glycosylceramide, glycoprotein antigens, or glycolipid antigen with or without SAg are presented on CD1 receptors, then NKT cells may generate the predominant T cell response. However the classical T cell system is also responsive. These constructs are produced and used as a vaccine against established tumor by protocols given in Examples 2-5, 15, 16 18-23.

23. Nucleic Acids Encoding CD1 Receptors

Nucleic acid encoding the CD1 receptor is transfected into tumor cells, resulting in expression of the CD1 receptor on the tumor cell surface. Promoters of CD1 synthesis are also useful in this invention. The human genome includes five CD1 genes (A-D) which also function in antigen presentation to T cells (Calabi, F et al., CD 1: From Structure to Function in Immunogenetics of the Major Histocompatibility Complex, Srivastava, R et al., eds, VCH publishers, New York, N.Y., 1991). In mice, two homologous proteins (mCD1.1 and 1.2) have been characterized and map to chromosome 3. The human CD1 genes are located on chromosome 1q221-q23 in the order D-A-C-E a from the centromere on a 190 kb segment of DNA. With the exception of CD1B, they are all in the same transcriptional orientation. They are evenly spaced in the complex with one exception: CD1D and CD1A are spaced two to three times farther apart than the average. The products of CD1A, -B and -C genes have been defined serologically. The products of CD1D and CD1E are unknown. They share a highly conserved exon domain which is homologous to the b2m-binding domain (a3) of MHC class I antigens. The CD1 molecules are not polymorphic and apart from CD1D, are noncovalently associated with a b2m in a TAP-independent manner. Complex alternative splicing of CD1 genes results in tissue specific forms of the protein, which can be intracellular, membrane bound, or secreted. In cells infected with mycobacteria, the CD1 molecule binds and presents a mycobacterial membrane component, mycolic acid. Surface CD1 molecules present longer peptides than those normally found on class I molecules. Whether CD1 molecules can also present peptide antigens is still unclear although this has been shown for at least one member of the CD1 family.

Tumor cells are transfected with nucleic acid encoding the CD1 receptor. Nucleic acid encoding cell wall or cell membrane associated glycosylceramides or a branched, b hydroxy long-chain fatty acids found in mycobacteria and other bacteria are cotransfected into the CD1 transfected tumor cells. The tumor cell therefore displays glycosylceramides bound to the CD1 receptor.

Using site directed mutagenesis, DNA encoding the CD1 receptor is provided along with DNA encoding a SAg binding site. This SAg binding site consists of key amino acids from the SAg receptor or from the SAg binding sites on (i) MHC class II chains or (ii) the TCR Vb region. This may consist of a glycosphingolipid sequence (sensitive to endoglycoceramidase) present on some mammalian cells.

The glycosylceramide used to bind to the CD1 receptor will have an exposed SAg binding site which is sensitive to endoglycoceramidase, an enzyme from Rhodococcus which specifically cleaves the glycosyl moiety from glycosphingolipids. Other ceramidases break up sphingolipid into fatty acids and sphingosine.

These tumor cells transfectants are prepared as in Examples 1 and 2. They are used in vivo as a preventative or therapeutic antitumor vaccine as in Example 14-16, 18-23. They are also useful ex vivo to produce a population of tumor specific T or NKT cells for adoptive immunotherapy of cancer (Example 2-5, 7, 15, 16, 18-23).

24. DNA Encoding Streptococcal M Proteins and DNA Encoding Protein A or Its Fc and VH3 IgG Binding Domains Transfected into Tumor Cells Alone or SAg DNA

The streptococcal M proteins are type-specific and act as protective or virulence factors. M protein genes are members of a larger emm-like gene family, such that many S. pyogenes strains express more than one M-like protein. DNA encoding the streptococcal M protein and DNA of the larger emm-like family are transfected into tumor cells (Kchoe M. A., “Cell-Wall Associated Proteins in Gram-Positive Bacteria,” In: Bacterial Cell Wall, Ghuysen J M et al., eds, Elsevier, Amsterdam, 1994).

In addition, DNA encoding protein A and its domains as well as DNA of the streptococcal fcrA76 gene located upstream of the emm-like gene are transfected into tumor cells individually or together to cause the expression of IgG FcR- and VH3 IgG-binding domains (Kehoe M A, supra). DNA encoding SAg is cotransfected into the same tumor cells to produce a tumor cell expressing any combination of M protein, protein A and a SAg.

Such cells are used in vivo as preventive vaccines or as therapeutic vaccines against established tumors. See Examples 1-5, 11, 15, 16, 18-23. They may also be used ex vivo to induce populations of active tumor specific effector T cells that are then used in adoptive immunotherapy See Examples 2-5, 7, 15-16, 18-23. 25.

Nucleic Acids, Bacterial Cells and Phage Displays Mimicking SAgs

Because of circulating naturally occurring antibodies in humans, native or mutated SAgs that are administered parenterally are not likely to reach the appropriate receptors on T cells or tumor cells. To solve this problem, mimic oligonucleotides are prepared—these mimic SAgs in their capacity to bind SAg receptors. Since no natural antibodies are directed to these compositions, they will not be prevented from reaching specific SAg receptors in vivo.

SAg receptors are used to screen oligonucleotides for their ability to mimic SAg binding. Useful receptors for such screening include those described herein (as expressed on tumor cells) and T cell TCR V chains. For example, pools of oligonucleotides are tested for their binding to, and affinity for, immobilized SAg receptors using nucleotide chromatography technology well known in the art. Once these high affinity binding oligonucleotides are identified, they are isolated (or, following sequencing, may be synthesized) and administered to a host.

Also included here is a bifunctional oligonucleotide-peptide chimeric molecule that binds specifically to the SAg receptor on tumor cells as well as the Vβ-36region of the TCR. Such an oligonucleotide will bind simultaneously to tumor cells and T cells (in the process of activation) to produce an anti-tumor response. An oligonucleotide-protein construct is prepared consisting of (a) a peptide sequence of enterotoxin A that binds to the TCR and (b) an oligonucleotide that binds to SAg receptor on tumor cells. The peptide portion of this construct should be devoid of MHC class II binding sites in order to minimize undesired binding of the molecule to class II structures upon administration in vivo.

In another embodiment, the nucleic acid portion of the chimeric molecule binds to the TCR while the peptide consists of a non-enterotoxin ligand that is specific for the SAg receptor on tumor cells. This construct has the advantage of lacking any binding site for natural antibodies. Yet another additional chimeric molecule consists of an oligonucleotide portion specific for the class II (or (chain and a second oligonucleotide or a peptide specific for the TCR Vβ chain.

Methods for preparing these constructs are given in Examples 5, 13. These constructs are especially useful for targeting tumors in vivo while also promoting a T cell anti-tumor response. See Examples 18-23. However, these chimeric molecules may also be used ex vivo in the production of tumor specific effector T cells capable of inducing, or effecting, an anti-tumor response when administered to a tumor bearing host. See protocols in Examples 2-5, 15, 16 18-23.

SAg and GlycosylCeramide Co-Expression

This may be accomplished using intact bacteria or phage display approaches. Since the precursors and substrates of the glycosyltransferases are not readily available in most mammalian cells, it is more convenient to induce dual expression of GalCer and SAgs in bacteria, for example Sphingomonas paucimobilis, which naturally produce GalCer. Hence, nucleic acid encoding a SAg is transfected into this bacterium together with a suitable promoter well known in the art. The bacterium produces both GalCer and SAg.

By ensuring that the SAg contains one or more glycosylation sites (by using the appropriate nucleic acid sequence), a glycosylated SAg is produced. Such a SAg binds to the glycosyl ceramide, e.g., GalCer to form a conjugate that is expressed on the bacterial surface of is secreted. In either form, such a SAg-GalCer conjugate can sensitize NKT cells to produce an anti-tumor response.

In addition, phage or plasmids encoding the appropriate transferase are transfected into low virulence Staphylococcus species which also produce enterotoxins. The bacterium acquires the capability of expressing GalCer on its surface. These bacterial constructs and compositions are used in vivo in a tumor bearing host to produce an anti-tumor response in protocols given in Examples 5, 13, 15, 16 18-23 and Detailed Description Section 19. They are also are used ex vivo to activate NKT cells or T cells to differentiate to tumor specific effector cells for use in adoptive immunotherapy of cancer by protocols in Example 1, 2, 14-16, 18-23).

Phage display technology is used to target selected SAg sequences to targets in vivo. The selected peptide is used as a binding sequences in lieu of the full-length polypeptide. This permits elimination from the construct of the antigenic portion of the SAg to which natural antibodies are directed. Cloned genes are expressed as part of phage coat proteins, for example, as fusions with the gene III protein (gIIIp) or the gene vIII protein (gVIIIp). In addition to the displayed gene product, the phage genome (of each particle) includes the gene encoding this product.

Phage display is preferably done using the filamentous phage f88-4 and comprises forming a fusion that results in the C terminus of the “selected” (i.e., inserted gene's) product and the N terminus of the phage protein gVIIp. Peptides of various enterotoxins are expressed in the phage display—most preferably peptides that bind to the SAg receptor on colon carcinoma cells. These peptides retain their capacity to bind to the TCR and to activate T cells. Also contemplated within this invention is phage display of SAg plus nucleic acid encoding synthesis of GalCer and/or the Gal epitope. DNA for synthesis of GalCer is preferably isolated from Sphingomonas paucimobilis; DNA encoding the galactosyl transferase for synthesis of Gal is preferably isolated from Klebsiella aerobacter, Serratia, E. coli and Salmonella organisms which naturally a produce and express these epitopes. The phage displays are administered in vivo and are capable of initiating a potent immune response to the tumor using the protocols described in Examples 5 and 13 and Section 19, above. These preparations are also useful for activating T cells or NKT cells ex vivo to produce a tumor specific effector cells for use in adoptive immunotherapy (Examples 2-5, 14-16, 18-23).

Viral infection of a host cell having the galactosyl transferase results in the shedding of virions that express the Gal epitope. When a host mammalian cell has been transfected with nucleic acid encoding SAg, the virus can coexpress the α-Gal epitope and the SAg on its surface. Such a viral construct is administered in vivo to achieve a therapeutic effect, or, in another embodiment, is employed ex vivo to produce tumor specific effector T or NKT cells for use in adoptive immunotherapy of cancer (Examples 2, 3, 7, 15, 16, 18-23).

26. Combining SAgs with Enterotoxin Precursors (Cell-Bound Dimers and Oligomers) and with Enterotoxin Promoters and Transcriptional Regulatory Genes

Cell-Bound SAg Dimers and Oligomers

Staphylococcal enterotoxins are present in the membrane of enterotoxin producing bacteria in dimeric form and retain potent enterotoxin-like activity when isolated from the membrane. It is in this membrane-bound form that enterotoxins are combined with tumor associated antigens or oncogene products and presented to the T cell system. The dimerization of the enterotoxins may promote clustering for more effective presentation to T cells. Indeed, dimerization or polymerization of enterotoxins or the introduction of tandem repeats of the SAg binding sites for TCR and MHC class II may be achieved by (1) site directed mutagenesis of the enterotoxin plasmid and (2) introduction of sequences for gene amplification, tandem repetition and/or recombination or by (3) introduction of enzymes for peptide chain elongation. The duplication may be at the level of the bacterial operon including its transcriptional regulators, using methods well described in the art. Modified plasmid is DNA is introduced into the target tumor cells or into accessory cells, either or both of which are useful in vivo as a preventative or therapeutic vaccine (Examples 1, 2, 15, 16, 18-23). Such genetically transformed cells may also be used ex vivo to produce effector T or NKT cells for adoptive immunotherapy (Examples 1, 2, 7, 15, 16, 18-23).

SAg agr Locus (Accessory Gene Regulator) and Other Bacterial Genes and Elements

At least 15 gene coding for potential virulence factors in S. aureus are regulated by a putative multicomponent signal transduction system encoded by the agr/hld locus. The synthesis of at least 14 exotoxins and enzymes in S. aureus is regulated by a set of transacting elements from agr. The agr gene coordinately controls the expression of exfoliatin toxin, toxic shock syndrome toxin, a, b, d toxins, enterotoxin B, lipases and nucleases (Balaban, N. et al., Proc. Natl. Acad. Sci. USA 92:1619-1623 (1995)). These proteins are members of the histidine protein kinase family of regulators and control a number of virulence determinants (Balaban supra, Novick R P, Meth Enzymol. 204: 587-637 (1991)).

Compared to wild-type, agr and hld mutants have decreased synthesis of extracellular a toxins and enzymes (such as a-, b-, and g-hemolysins, leucocidin lipase, hyaluronate lyase and proteases) while having increased synthesis of coagulase and protein A. The agr gene consists of two divergent transcriptional units driven by promoters named P2 and P3. The P2 transcript includes four open reading frames referred to as agrA, B, C, and D, all four of which are required to for the agr response.

The peptides predicted for agrA and agrC resemble the response regulators and signal transducers of the two-component bacterial signal transduction systems. The primary function of thee four genes discussed above is to activate two promoters; the P3 transcript, RNAIII, however is the actual effector of the exotoxin response. RNAIII activates transcription of secretory protein genes and represses transcriptions of surface protein genes. As a global regulatory system, agr, controls the post-exponential production of exoproteins such as toxins, hemolysins, and exoenzymes. agr is a complex polycistronic locus that encodes a two-component signal transduction pathway that activates transcription of a regulatory RNA molecule that in turn activates transcription of the exoprotein genes.

Thus, transcriptional regulation of the enterotoxin B gene as well as SED, SEC and staphylococcal capsular polysaccharide gene involves the agr product. (agr does not regulate SEA expression).

The promoter region of SEA is localized by primer extension analysis. The 5′-end of SEA mRNA is localized 86 bp upstream of the translational initiation codon. A DNA region with good agreement with canonical promoter sequences was observed beginning 8 base pairs upstream of the apparent transcriptional start site. No DNA upstream of the 35 bp region is required for transcription. Both the agr gene and the SEA promoter have been cloned (Peng, H. L. et al., J. Bacteriol. 170:4365-4372 (1988); Borst, D. W. et al., Infec. Immun. 61:5421-5425 (1993)). The xpr locus and the agr locus interact at the genotypic level; agr is autoinduced by a proteinaceous factor produced and secreted by the bacteria and is inhibited by a peptide from an exotoxin-deficient S. aureus mutant strain. The inhibitor, RIP, competes with the activator, RAP. When given as a vaccine, RIP may prove useful as a direct inhibitor of virulence.

A chromosomal locus (sar) distinct from agr, encodes a DNA-binding protein that is important in regulation, and is required for expression of S. aureus exoproteins including enterotoxin, toxic shock syndrome toxin, hemolysin and staphylokinase. Transcription of Protein A is suppressed by sar and agr.

A list of plasmids containing bacterial virulence factors useful in this invention is disclosed in Table 49, p. 223 of Patrick, S. et al., Immunological and Molecular Aspects of Bacterial Virulence, John Wiley and Son, New York, N.Y. 1995. This invention contemplates the use of the Staphylococcal enterotoxin promoters and transcription factors that activate the enterotoxin biosynthetic cycle. Several Staphylococcal promoters have been identified (Novick, supra). This invention also contemplates the use of the peptide activator RAP which induces agr as well as the peptide inhibitor RIP which induces or represses RNA III.

SAg-encoding nucleic acid is fused in-frame with Staphylococcus agr nucleic acid and introduced into tumor cells or accessory cells (or the two are cotransfected into these cells). In another embodiment, SAg-encoding nucleic acids placed under the control of an enterotoxin promoter, and this construct is introduced into tumor cells or accessory cells. The agr gene is especially useful because it can be linked to an inducible promoter such as that for corticosteroids or the metallothionein promoter, allowing it to be activated in a controlled manner by exogenous administration of the inducing to the host.

Methods for introducing the above genes into tumor cells are described in Example 1, 2, 11. The use of such cells in vivo as preventative or therapeutic vaccines are discussed in Examples 15, 16, 18-23. Use of these genetically transformed tumor cells ex vivo to induce effector T or NKT cells for adoptive immunotherapy is described in Examples 2, 3, 7, 15, 16, 18-23.

27. Combining SAg with Oncogenes, Protooncogenes, Amplified Oncogenes, Transcription Factors or Tumor Markers

In one embodiment, the nucleic acid encoding a SAg is fused in-frame to oncogene or protooncogene nucleic acid in tumor cells or accessory cells to produce a chimeric nucleic acid which is expressed in, or on the surface of, the cell. This fused gene may be rendered inducible by judicious choice of a promoter or other regulatory sequence. Preferably, such an inducible promoter is induced by a hormone or a metal. A regulatory element, such as one activated by interferon or a cytokine (e.g., Jak or a STAT), may be included in this construct.

In another embodiment, the nucleic acid encoding SAg is fused in frame to nucleic acid encoding an oncogene which can be amplified markedly. The fused construct is introduced into tumor cells or accessory cells. An amplified “unit” is initially much larger than the size of the actual gene of importance to the oncogenic event(s) (Hellems, R E, Gene Amplification in Mammalian Cells, Marcel Dekker, New York, N.Y. ). Thus a silent gene is co-amplified with one or more genes expressed on an amplicon. This is a preferred site for the inserting gene clusters wherein one gene encodes a SAg, others encode the enzymes of LPS lipid A biosynthesis, optionally together with their native promoters or operons.

Transcription Factors and Amplified Oncogenes

Oncogenes are frequently amplified in human tumors and cultured cancer cells. This is more characteristic of solid tumors and relatively rare in lymphoid malignancies. DNA amplification was first observed cytogenetically a double minute chromosomes (DMs) or homogeneously staining regions (HSRs) but today, direct DNA analysis (Southern blotting) or molecular cytogenetic methodologies, such as fluorescence in situ hybridization (FISH) and comparative genomic hybridization (CGH) can be applied. DMs are episomal forms of amplified DNA that generally lack centromeres and are unequally distributed between daughter cells at mitosis. They appear as isodiametric extrachromosomal bodies stainable with all chromatin dyes. HSRs are chromosomally integrated forms of amplified DNA. They represent either the replacement of the normal chromosome banding pattern with an extended region of homogenous staining or the insertion of such a region into an otherwise normally banded chromosome. DMs and HSRs tend to be mutually exclusive and are potentially interchangeable manifestations of the amplified DNA. Thus, DMs can potentially integrate into distant chromosomal sites to generate heritable HSR. Of 22 human tumors analyzed, 91% contained DMs only, 6.5% contained HSRs and 2.5% contained both. In solid tumors of epithelial origin, DMs and HSR were found in 40% of breast carcinomas, 17% of non small cell carcinoma of the lung, 18% of stomach and esophageal cancers and 15% of uterine carcinomas.

The overwhelming majority of oncogene amplifications in human tumors affect the Myc oncogene family. In small cell lung cancers all three members of the Myc family, c-myc, N-myc and L-myc can be involved. Myc amplification is associated with a more invasive and more metastatic phenotype. N-myc amplification is seen in neuroblastoma and is associated with the late stages and poor prognosis. The amplification units on chromosome 11q13 are seen in (a) breast cancer, (b) squamous cell carcinoma of the head and neck, lung, and esophagus and (c) bladder tumors. The amplification extends for over 1.5 megabase pairs of DNA and includes two bona fide oncogenes: FGF3 and FGF4. It also includes the Bcl-1 CCND1 (cyclin D1) as well as the EMS1 gene that encodes the human homologue of cortactin. CCND1 has a critical role in amplified DNA since its expression is increased as a consequence of amplification. The other major targets for amplification are the genes encoding the EGF receptor (ErbB1/Her1) and the related ErbB2/Her2. Both genes are amplified in breast cancer and other malignancies. ErbB2 is associated with estrogen receptor-negative breast cancers and poor prognosis.

Members of the myc gene family are activated in several human tumors as a result of DNA rearrangements through chromosomal translocations or gene amplification. When overexpressed, all myc genes complement mutant c-ras oncogenes in the transformation of primary rat embryonic cells and transform Rat 1-A cells without assistance of other oncogenes. Stimulation of cellular myc expression levels or changes in post-translational modification of myc proteins have been following exposure of cells to many growth promoting stimuli. These features suggest that the myc proteins participate in the final steps of mitogenic signal transduction. The myc proteins act as transcription factors involved in activation and/or repression of target genes. In neuroblastoma, a group whose tumors are generally near diploid or tetraploid with chromosome 1p deletion(LOH) and N-myc amplification have a generally poor response to treatment and a poor prognosis. Genomic amplification of the N-myc cellular oncogene is present in approximately 40% of cases of childhood neuroblastoma and correlates with histopathological signs of advanced disease. This genomic N-myc amplification appears to be associated with tumor progression rather than tumor initiation since early stage tumors rarely exhibit M-myc genomic amplification. Similarly the c-myc family of protooncogenes including N-myc and L-myc are amplified in small cell carcinoma of the lung.

The amplified oncogenes useful in the present invention include genes encoding transcription factors. The preferred nucleic acids for use in the present invention are c-myc, N-myc, c-abl, c-myb, c-erb, c-Ki-ras, N-ras. N-myc (amplified 5-1000 fold in neuroblastoma) is preferred. SAg-encoding nucleic acid is cotransfected into tumor cells or accessory cells with amplified oncogenes. The N-myc and L-myc genes have been cloned as c-myc homologous amplified oncogenes from human tumors. In one embodiment, SAg-encoding nucleic acid is fused in frame with nucleic acid encoding oncogenic transcription factors such as FOS, JUN. MYC, MYB and ETS. In another embodiment, such nucleic acid is cotransfected with SAg-encoding nucleic acids. Either of such constructs is introduced into tumor cells or accessory cells. Proteins that interact with FOS and JUN are given in Table 1 p.157 of Peters G et al., Oncogenes and Tumor Suppressors, Oxford University Press, Oxford UK 1997, incorporated by reference.

bcr/abl Gene

SAg-encoding nucleic acid is fused in frame or cotransfected with nucleic acids encoding the following agents and transfected into tumor cells and fused to oncogenic nucleic acids encoding chimeric proteins capable of immunizing the tumor bearing host. An ideal candidates for such fusions is the bcr-abl gene which express the bcr/abl protein in chronic myelogenous leukemia (CML). The c-abl oncogene is amplified in chronic myelogenous leukemia. Scherle P A et al., Proc. Natl. Acad. Sci. USA 87: 1908-1917 (1990) Heisterkamp N et al., Nature 344: 251-253 (1990). Abnormalities in the structure and expression of the human c-abl cellular oncogene have been associated with

Philadelphia chromosome-positive CML which is present in more than 90% of cases. This aberrant chromosome marker is generated by a reciprocal translocation between chromosomes 9 and 22 in which the c-abl oncogene is translocated from the distal end of the q arm of chromosome 9 to a relatively restricted 5-6 kb region on chromosome 22 termed the breakpoint cluster region (bcr). This translocation creates a fusion gene that is transcribed as an 8 kb bcr-abl RNA that encodes the aberrant bcr-abl fusion protein product (P210) observed in CML cells. The bcr-abl fusion product has enhanced tyrosine kinase activity compared with the normal p145 c-abl product. Abnormalities in the structure and expression of the c-abl cellular oncogene have not been described in any type of human malignancy other than CML and Ph positive acute lymphatic leukemia. Gene amplification correlates with progression of malignancy.

EGF Receptor Genes

SAg-encoding nucleic acid is fused in frame to the nucleic acids encoding the EGF receptor (EGFR) (Ulrich A et al., Nature 309: 418-421 (1984)). The EGFR is the prototype of four-member receptor family. EGFR is frequently overexpressed or mutated in several different types of human tumor. For instance, the EGFR is amplified in 20-40% of human glioblastomas and a variety of epithelial tumors including head and neck squamous cell carcinomas, breast tumors, esophageal tumors and urogenital tumors. Amplification was accompanied by overexpression of the EGFR.

The erbB2 (her2/neu) Oncogene

SAg-encoding DNA is fused in frame to DNA encoding a tumor marker such as PSA, c-erbB2(neu), her2/neu, bcl-2 and Brea-1. The principal amplified and functional genes in breast cancer are the growth factor receptor-erbB2, the nuclear transcription factor c-myc, and the genes encoding cell cycle kinase regulatory genes termed cyclin D1 and cyclin EG. Gene amplification is thought to proceed via the initial formation of extrachromosomal, self replicating units (double minute chromosomes) that become permanently incorporated into chromosomal regions where they are called homogeneously staining regions (HSRs) as described above. The human counterpart of the oncogene neu known as her2 encodes a protein of the same family as the EGFR. This family of genes has been cloned. Its products belong to a family of receptor tyrosine kinases each with a transmembrane domain, a cysteine-rich extracellular domain and an intracellular catalytic domain. They act as receptors for several peptide growth factors such as EGF, TGF( and neuregulins. The activated receptors are then able to bind to proteins containing src-homology-2 (SH2) domains. The SH2 domain proteins recognize and bind to specific phosphotyrosine-containing sequences of the activated receptor. These SH2 containing adapter molecules then trigger downstream signalling pathways, ultimately resulting in gene activation.

The erbB2 (neu/Her2) gene maps to chromosome 17p21 and codes for a 185 kDa transmembrane glycoprotein related to, but not identical to the EGF receptor (Schechter A L et al., Science 229: 976-978 (1985), Bargmann C L Nature 319: 226-230 (1986), Hung M C et al., Proc. Natl. Acad. Sci. USA 83: 261-264 (1986), Yamamoto T et al., Nature 319: 230-234 (1986)). The EGFR bears sequence homology with the erbB1 product. The erbB2 gene is activated by a point mutation which mutates amino acid residue 664 from valine to glutamic acid; this change is associated with transforming its ability. The genes are called erbB, (erbB1 , EGFr), erbB2, (neu/Her-2). erbB3(HER-3) and erb B4 (HER-4).

Amplification and overexpression of erbB2 has been found in a variety of human tumors including carcinomas of the breast, ovaries, colon, lung, liver, stomach, kidney, esophagus, salivary gland, and bladder. Genomic amplification of the neu (C-erb-2) or HER2 cellular oncogene and protein overexpression has been documented in approximately 30% of primary human breast cancers and may correlate with advanced disease and a relatively poor prognosis. More than 50% of all ductal carcinomas in situ of the large cell type express HER2. Amplification occurs in approximately 20% of invasive breast carcinomas. Thus, it is thought that HER2 amplification increases the growth rate but not the metastatic potential of tumor cells.

A third member of the EGFR family is ERBB3which is present in some human breast cancers with high expression correlating with lymph node metastases. Overexpression of

ERBB3 has been observed in epidermoid carcinoma of the larynx and esophageal carcinoma. ERBB4, a fourth member of the EGFR family, was overexpressed in a human mammary tumor cell line.Fisk et al. (J. Exp. Med. 181: 2109-2117 (1995)) described an immunodominant epitope of HER/neu that is recognized by ovarian tumor-specific cytotoxic T lymphocytes. This epitope is useful in this invention. Failure of coexpression of a heterodimeric partner or coinduction of a suppressor phosphatase would explain the lack of immunogenicity of c-erbB2 in mice in nude mice.

Additional oncogenes, protooncogenes and tumor markers which would be candidates for the fusion in accordance with this invention are w PSA, c-erb B2(neu), Her2/neu, bcl-2, Brca-1. Viral and non-viral oncogenes and protooncogenes which are overexpressed in tumor cells are shown in Table 9.2 and 9.1, p. 171-172 of Franks et al., supra). The functions of the various oncogenes is shown in Table 9.6, p.186, of Franks et al.

IGF Receptor Genes

SAg-encoding nucleic acid is fused in frame with nucleic acids encoding insulin-like growth factor (IGF) receptors (IGFRs)and transfected into tumor cells. The IGFR gene is a tyrosine kinase containing transmembrane protein that plays an important role in cell growth control. There is a single IGF1 receptor gene with a complete coding sequence contained in 21 exons (Abbott A M et al., J. Biol. Chem. 267: 10759-10763 (1992); Scott J et al., Nature 317: 260-262 (1985); Liu J et al., Cell 75: 59-63 (1993)).

IGF1R is expressed at high levels in breast cancer, and amplification of the IGF1R gene has been observed. IGFs play a significant auxiliary role in tumor growth by suppression of apoptosis. The apoptotic effect overexpressed myc is overcome by IGFs. Thus, IGFs facilitate tumor growth by suppression of apoptosis.

Fibroblast Growth Factor (FGF) Receptor Genes

SAg-encoding nucleic acid is fused in frame to nucleic acids encoding fibroblast growth factors receptors (FGFs) and transfected into tumor cells. FGF receptor are also be important for the vascularization of certain types of tumors. The expression of FGF1 has been shown to be associated with a switch to an angiogenic phenotype during the development of a fibrosarcoma. Overexpression of FGF receptor by certain tumors may also contribute to their growth. FGF receptors have been shown to be amplified in some breast cancers.

Platelet Derived Growth Factor (PDGF) Receptor Genes

SAg-encoding nucleic acid is fused in frame to nucleic acids encoding additional tumor growth factors which are produced or overexpressed and transfected into tumor cells or accessory cells. Growth factors include those in the tyrosine kinase receptor families such as Platelet Derived Growth Factor A and B family (PDGF). PDGF A and B receptors are amplified in malignant glioblastomas in the malignant cells themselves or the stromal cells (Fleming T P et al., Cancer Res. 52: 4550-4556 (1992);Kumabe T et al., Oncogene 7: 627-632 (1992)). The nerve growth factor (NGF), stem cell factor receptor (kit), colony stimulating factor-1 receptor (fms), neurotropin receptor family, transforming growth factor b family, the WNT family, angiogenic receptors

Other Amplified Oncogenes

SAg-encoding nucleic acid is also fused to nucleic acid encoding the tyrosine protein kinases which are both membrane associated and transmembrane as described in Table 9.4, p. 179 of Franks et al., supra. Additional chromosomal regions which are amplified in greater than 40% of cases included the 8q24 locus of the c-myc(O) gene, the 11q13 locus of the cyclin D (O), int2 (O), EMS-1 (O), BCL-1 (O), FGF-4 (O) GST (M), MEN1 (S) genes, the 17q21 locus of the RARa (S), RARg (S), ERBAa (S), BRCA1 (S), NM23 (S), estradiol 17B dehydrogenase (S) ERG2 (O), HOX2, NGFR (O), WNT3 (O) and the 20q13 locus.

Nucleic acid encoding SAg is fused or cotransfected into tumor cells with nucleic acid encoding the above oncogenes, amplified oncogenes and protooncogenes, transcription factors and growth factor receptors. These transfectants are prepared as in Examples 1. They are useful in vivo as a preventative or therapeutic vaccine (Examples 15, 16, 18-23). They are also useful ex vivo for inducing tumor specific effector cells for adoptive immunotherapy (Examples 2-5, 7, 15, 16 18-23).

28. Combining SAg with Angiogenic Receptors and Growth Factor Receptors

SAg-encoding nucleic acid is cotransfected or fused in frame to nucleic acid encoding an angiogenic receptor such as VEGF and transfected into tumor cells. SAg nucleic acid is also fused to or cotransfected with nucleic acid encoding other angiogenic receptors such as V integrin, other integrins, cadherins or selectins and introduced into tumor cells or accessory cells. SAg-encoding nucleic acid is also cotransfected into tumor cells or accessory cells with nucleic acids encoding angiogenic proteins such as VEGF.

VEGF is produced by tumor cells and stroma, and its expression correlates with the degree of vascularization and grade of malignancy. VEGF receptors, termed KDR and flt, are expressed mainly by the tumor endothelium. Higher levels of VEGF are found in metastatic than in non-metastatic colon cancers (Tischer E et al., J. Biol. Chem. 266: 11947-11954 (1991). VEGF is especially useful here because it is overexpressed in tumor cells at an early stage of tumorigenesis. The promoter of the VEGF gene lacks a TATA box, but has six GC boxes for transcription factor SP-1 binding and also a site for AP-1 and AP-2 binding. The expression of the gene is modulated by several growth factors such as EGF. In some cell types VEGF expression is regulated by IL-1, FGF, PDGF. A common element, mediation of protein kinase C in the regulation of VEGF, has been suggested. VEGF is expressed as a disulfide linked dimer. Long and short forms are generated by alternative splicing and are matrix bound or released, respectively. As a result of its specific effects on endothelial cell migration and proliferation, VEGF is a very potent and specific promoter of angiogenesis. Two well characterized families of angiogenic factors act by binding to tyrosine kinase receptors that have two or three immunoglobulin-like domains, and VEGF binds to two related receptors with seven immunoglobulin-like extracellular domains.

The TRKA oncogene codes for a receptor for nerve growth factor (NGF). The TRKA gene has been found fused to genes that code for proteins that form dimers in cells leading to the synthesis of a constitutively dimerized and active tyrosine kinase. TRKA may have a tumor suppressor function since its expression in neuroblastoma correlated inversely with n-myc gene amplification. Coexpression of mRNA for TRKA and the low affinity NGF receptor in neuroblastoma correlated with a favorable prognosis. Nucleic acid encoding SAg is fused to nucleic acid encoding the above angiogenic factors or receptors and introduced into tumor cells; alternatively, the two nucleic acids are used to cotransfected tumor cells. These transfectants are prepared as in Example 1. They are useful in vivo as a preventative or therapeutic antitumor vaccine (Examples 15, 16, 18-23). They are also useful ex vivo for inducing tumor specific effector cells for adoptive immunotherapy of cancer (Examples 2-5, 7, 15.16 18-23).

29. Combination of SAg with Cell Cycle Protein

SAg-encoding nucleic acid is fused in frame to nucleic acid encoding a cell cycle protein such as a cyclin which is overexpressed in tumor cells. Examples of these cell cycle proteins which are preferred for such fusions are Cyclins A, B, D1, E. These proteins are generally complexed to kinases or transcription factors at critical checkpoints in the cell cycle. The cyclins, CDKs and their inhibitors are shown in Table 1. p193 of Peters G et al., supra.

In another embodiment, nucleic acid encoding SAg is cotransfected into tumor cells with nucleic acid encoding a cell cycle protein as above. These transfectants are prepared as in Examples 1. They are useful in vivo as a preventative or therapeutic antitumor vaccines (Examples 15, 16, 18-23). They are also useful ex vivo for inducing tumor a specific effector cells for adoptive immunotherapy of cancer (Examples 2-5, 7, 15.16 18-23).

30. Combining SAg with Tumor Suppressor Genes, p53 or Developmental Genes

SAg-encoding nucleic acid is fused in frame with tumor suppressor gene DNA and the fused nucleic acid is introduced into tumor cells or accessory cells. Alternatively, the two nucleic acids are used to cotransfected these cells. Examples of such tumor suppressor genes are shown in Table 9.7 p.187 of Franks L M et al., supra Examples of mutated tumor suppressor genes include the APC and MCC genes and their isoforms, the DCC gene in colon cancer, the BRCA1 tumor suppressor gene in breast cancer and the DPC gene in pancreatic cancer. The p53 gene and its mutations are also useful in this embodiment. A list of p53 responsive elements and associated proteins useful in this invention is given in Tables 1 and 2 pp. 267-269 of Peters G et al., supra.

In another embodiment, nucleic acid of developmental genes is used in place of tumor suppressor or p53 genes. Examples of such developmental or differentiation genes are wnt and fwt genes.

Transfectants are prepared as in Examples 1. They are useful in vivo as a preventative or therapeutic antitumor vaccine according to Examples 15, 16, 18-23). They are also useful ex vivo for inducing tumor specific effector cells for adoptive immunotherapy of cancer (Examples 2-5, 7, 15, 16, 18-23).

31. Combining SAg with Cell Surface Glycoproteins or Their Receptors

SAg-encoding nucleic acid is fused in frame with a nucleic acid encoding a cell surface glycoprotein and or its receptor and the fused nucleic acid is introduced into tumor cells or accessory cells. Alternatively, the two nucleic acids are used to cotransfected these cells. Examples of these glycoproteins or receptors include integrins, vitronectin receptors, laminin receptors, cadherins, tenascin and CD44 and isoforms, VCAM-1, P-Selectins, E-Selectin, NCAM and MCAM. Transfectants are prepared as in Example 1. They are useful in vivo as a preventative or therapeutic antitumor vaccine according to Examples 15, 16, 18-23). They are also useful ex vivo for inducing tumor specific effector cells for adoptive immunotherapy of cancer (Examples 2-5, 7, 15, 16 18-23).

32. Combining SAg with Cytokines and Chemokines

SAg-encoding nucleic acid is fused in frame with nucleic acid encoding a cytokines and chemokines, and the fused nucleic acid is introduced into tumor cells or accessory cells. Alternatively, the two nucleic acids are used to cotransfected these cells. Examples of chemokines and cytokines that are useful herein include RANTES, IL-5, IL-7, IL-12, IL-13, IFNγ, TNFα and TNF β. Chemokines are small (typically 6-10 kDa) peptides that have been divided into two classes designated C—C and CXC based on the sequence of the first two cysteine residues. The two families exhibit preferences for different target cell types: C—C chemokines act primarily on macrophages.

Chemokine gene expression is induced by the action of other growth factors and cytokines and are actively expressed in solid tumors showing inflammatory involvement and macrophage or neutrophil invasion. Chemokines of the C—X—C class containing the amino acid sequence motif ELR have demonstrable angiogenic activity which can be inhibited by C—X—C chemokines lacking the ELR motif. Therefore chemokine expression by either tumor cells themselves or elicited from stromal cells by the action tumor-derived growth factors, have the potential to regulate tumor growth by modulation of angiogenesis. G-CSF is a growth factor for granulocyte precursors, and IL-2 is a growth factor for T cells.

Nucleic acids encoding SAgs are fused or cotransfected into tumor cells with nucleic acids encoding the above cytokines, chemokines and chemoattractants. The transfectants are prepared as in Example 1. They are useful in vivo as a preventative or therapeutic antitumor vaccine according to Examples 15, 16, 18-23). They are also useful ex vivo for inducing tumor specific effector cells for adoptive immunotherapy of cancer (Examples 2-5, 7, 15,16 18-23

33. Combining SAg with Transcription Factors AP-1 and NFkA

Transcription factor genes may act as oncogenes. The jun family of transcription factors bind specifically to AP-1 sites which confer the effects of potent tumor promoting phorbol esters on responsive genes and specifically bind to c-jun homodimers or c-jun/c-fos heterodimers. v-rel encodes members of the NF-kB family of transcription factors. Transforming oncogenes such as v-ets and v-myb also encode transcription factors.

The T cell signaling system responding to SAgs activates the JAK, TNF (TRAF), IL-2 and IL-12 pathway probably via NFkA activation. LPS has a T cell stimulating effect and may fuse with SAg to produce additional stimulation or epitope expansion. The NFA nucleic acids are fused to a promoter which activates sequences encoding the SAg receptor or the sequences encoding the key Vb domains binding SAgs or regions in the Vb receptor which are activated by the SAgs.

SAg-encoding nucleic acid is fused in frame with nucleic acids encoding a transcription factor such as those above. Transfectants are prepared as in Example 1. These transfectants are prepared as in Example 1. They are used in vivo as a preventative or therapeutic antitumor vaccine according to Examples 15, 16, 18-23). They are also used ex vivo for inducing tumor specific effector cells for adoptive immunotherapy of cancer (Examples 2-5, 7, 15, 16 18-23).)

34. SAgs Augment the Immunostimulatory Effects of Tumor Associated Peptides, Binary and Ternary Complexes

Bacterial SAg are presented to T cells via the MHC class II molecule by multiple low affinity attachments, resulting in stimulation of the T cell with very low concentrations of antigen. SAgs augment the presentation of antigenic peptides to T cells without sterically interfering with each other's ability to bind and activate the TCR. These augmenting peptides are incorporated into the SAg structure. SAgs may also bind to binary or ternary complexes of tumor peptide-MHC class I or tumor peptide-MHC class II complexes, either in solution or affixed to a TCR or the surface of an APC.

In one embodiment, the SAg is first bound to APCs or T cells followed by addition of complexes between MHC class I or class II and tumor peptide. Alternatively, the SAg may first bind to either cell-bound, soluble or immobilized MHC class I or class II molecules, after which the tumor peptide is added. This trimolecular complex is then presented to the T cell via the TCR.

In another embodiment, SAg is first bound to an APC or to a TCR Vβ chain on an NKT cell. Following this, CD1-glycosylceramide complexes are added and allowed to bind to NKT cell TCR Vβ chain. SAg may be bound to first to CD1-glycosylceramide complexes in soluble form, affixed to CD1+ cells or NKT cells via the TCR. SAgs may be bound to CD1 complexes with glycosylceramide or a glycosphingolipid (with a βconserved SAg binding site) in solution or when fixed to CD1+ cells or NKT cells. Alternatively, SAgs are bound to ternary complexes consisting of CD1-glycosylceramide affixed to the NKT cell TCR or bound to CD1-glycosylceramide on APCs, in solution or immobilized, before it has affixed to the NKT TCR. SAg is alternatively bound to binary complexes of (a) CD1-glycosylceramide, (b) CD1-glycosphingolipid, (c) CD14-LPS or (d) MHC-tumor peptide complexes that have either a SAg receptor sequence or a TCR Vb SAg-binding sequence.

The complexes described above are used in vivo as preventative or therapeutic antitumor vaccines according to Examples 4, 15, 16, 18-23. They are also used ex vivo for inducing tumor specific effector cells that are then taken for adoptive immunotherapy of cancer. (See Examples 2-5, 7, 14, 15, 16 18-23).

35. SAgs Combined with Products of Antigen Processing Pathways

A chimeric gene is prepared consisting of SAg-encoding nucleic acid fused in frame to nucleic acids encoding (a) the endoplasmic reticulum (ER) translocation signal peptide, (b) transmembrane domain, and (c) lysosomal targeting domain of LAMP-1. LAMP-1 is a type 1 transmembrane protein localized predominantly to lysosomes and late endosomes. The cytoplasmic domain of LAMP-1 contains the Tyr-Gln-Thr-Ile sequence that mediates the targeting of LAMP-1 into the endosomal and lysosomal compartments. The specific targeting of the SAg to the endosomal and lysosomal compartments allows SAg peptides to complex with MHC class II molecules and enhance presentation.

The MHC class I presentation pathway operates on a three level system. At one level there is protein machinery dedicated to peptide manufacture—the proteosome complex. The selective peptide transporters deliver antigens into the ER. The class I molecules themselves exhibit variable affinities for peptides. Genes clustered in the region of the class II gene encode proteosome and transporter. SAg peptides are transported into the ER—primarily through a transmembrane “tube” consisting of two polypeptide chains called TAP-1 (SEQ ID NOS: 40-41) and TAP-2 (transporter associated with antigen processing). In mammals, genes encoding TAP-1, TAP-2 and two proteosome polypeptides are all located within the class II region of the MHC.

The class I pathway starts in the cytosol where proteins produced inside the cell are degraded by the multicatalytic proteosome complex. The peptide products are translocated into the ER by the TAP proteins. In the lumen of the ER, the peptides bind the class I protein groove while the latter are complexed with the chaperone p88, b2m and TAP. After securing a peptide in its binding groove, the class I complex is released from TAP and transported through the Golgi apparatus to the cell surface. TAP genes are closely linked to the LMP2 (SEQ ID NOS: 38-39) and LMP7 in the class II MHC gene cluster and belong to a family of molecules involved in ATP-dependent membrane translocation known as the ABC (ATP-binding cassette) transporters. TAP1 and TAP2 function as a heterodimer each subunit having over 500 amino acids each with two hydrophobic domains, six membrane spanning regions and a cytosolic ATP binding motif. Both TAP1 and TAP2 subunits are required for peptide binding and translocation. TAP1 appears to be uniquely involved in the interactions with class I/β₂M dimers at the luminal membrane of the ER where it interacts with the membrane proximal region of the α 3 domain of class I-, β₂M complexes prior to peptide loading. Interaction between class I and TAP is crucial for efficient peptide loading. Antigen presentation is mediated by an additional factor, tapasin. TAP also binds β₂M independently of class I heavy chain, perhaps facilitating rapid assembly of class I peptide -binding complexes. TAP heterodimer may show a preference for amphipathic molecules as T cell antigenic determinants are often seen clustered around sequences where amphipathic helical structures are predicted. TAP prefers peptides 8-10 residues in length but may transport peptides ranging from 7-40 residues.

Invariant chains are transmembrane glycoproteins found in intracellular compartments in association with class II molecules. Multimers consisting of three class I α βdimers and three invariant chains assemble rapidly in the ER and travel across Golgi bodies to the trans-Golgi network that intersects with the endocytic pathway, where class II molecules reside for about 1-3 hr before transit to the cell surface for display to T cells. Alternative splicing of the invariant transcripts produces two isoforms p31 and p41 both of which can operate to assist folding of class II dimers, direct the passage of class II from the ER through an exocytic pathway, and block loading of peptide until peptide sampling can occur as exocytic-endocytic pathways intersect. A four residue targeting signal at the N-terminus of the invariant chain that is essential for intracellular transport to endosomal compartments. The C-terminus and the transmembrane region or the invariant chain are also necessary for sorting of class II-invariant chain complexes to the endosome. p41 appears to regulate the production of a stable 12-kDa SLIP-class II complex capable of enhancing SAg presentation.

SAg-encoding nucleic acid is fused in frame with nucleic acid encoding a protein involved in the antigen processing pathway such as the invariant chain or TAP which facilitates the expression of the SAg in the context of MHC class I and II, respectively. Tumor cells, accessory cells and hybrids thereof are transfected with fused SAg-invariant chain DNA as in Examples 1 and 5. They are used in vivo as a preventative or therapeutic antitumor vaccine according to Examples 15, 16, 18-23. They are also used ex vivo for inducing tumor specific effector cells for adoptive immunotherapy of cancer (Examples 2-5, 7, 15, 16 18-23).)

SAg polypeptide post translationally is fused or associated with additional molecules such as mono and diglycosylceramides, including but not limited to—anomeric mono- and digalactosylceramides GalCer, α-Gal, glycosylated and prenylated SAgs. These constructs translocate with the appropriate trafficking molecule e.g., invariant chain, TAP, LMP, to selected surface receptor such as MHC class I, MHC class II or CD1. These transfectants are prepared as in Example 1. They are useful in vivo as a preventative or therapeutic antitumor vaccine according to Examples 15, 16, 18-23. They are also useful ex vivo for inducing tumor specific effector cells for adoptive immunotherapy of cancer (Examples 2-5, 7, 15.16 18-23).

36. SAgs Combined with Signal Transduction Molecules or Heat Shock Proteins (HSPs)

SAg-encoding nucleic acid is fused in frame to (or cotransfected with) a nucleic acid encoding “signal transduction molecules” such as Ras, JAK 1 and STAT-1a and heat shock proteins HSP-60, HSP-70, HSP-90a, HSP-90b, Cox-2 as well as heterotrimeric G proteins and ATPases. The genes for Staphylococcal HSP-70 useful in this invention have been cloned (Ohta, T et al., J. Bacteriology 176: 4779-4783, (1994)). As used herein, SAg polypeptides are ligated to any of above structures at the peptide or nucleic acid level. Preferred proteins for this embodiment are G proteins, ATPases and HSPs. Chemical conjugation is carried out by conventional methods, e.g., use of preferred heterobifunctional crosslinkers. Alternatively, conjugates are produced genetically as fusion proteins by conventional methods. In yet another embodiment, the conjugates are created by permitting natural binding of the components to each other without chemical modification. Any of the foregoing conjugates or fusion proteins may be used when incorporated into vesicles or exosomes secreted from a cell. See Example 36 for methods and protocols.

SAg-encoding nucleic acid is fused in frame (or cotransfected) with nucleic acid encoding a signal transduction protein or HSP. Transfectants are prepared as in Example 1. They are used in vivo as a preventative or therapeutic antitumor vaccine according to Examples 15, 16, 18-23. They are also used ex vivo for inducing tumor specific effector cells for adoptive immunotherapy of cancer (Examples 2-5, 7, 15, 16 18-23). The peptide or polypeptide conjugates are also useful for the same purposes.

37. SAgs with Specialized Sites for C-terminal GPI Anchoring, Glycosylation, Sulfation, N-Myristoylation, Phosphorylation, Hydroxylation N-Methylation, Signal Peptide Binding, LPS Binding, HSP Binding, Chemokine Binding and Prenylation

SAg-encoding nucleic acid is fused in frame to nucleic acids encoding the above “specialized sites” and transfected into tumor cells or accessory cells The structures of these sites is given in Table 3, p. 48 of Rocker RBI et al., J. Nutrition 123: 977-990 (1993).

Tumor or accessory cells express SAgs in a variety of fashions after post-translational modification (Wilkins, M R. et al., Proteome Research: New Frontiers in Functional Genomics Springer. Berlin, Germany (1997)). For example, myristoylated SAg will a bind to surface lipids and will be minimally secreted. In glycosylated form, the SAg will be routed to the class II pathway and appear bound on the cell surface. When bound to invariant chain, the SAg will be routed to the class II receptor.

Nucleic acids encoding proteins that active in post-translational modification of SAgs are fused in frame to nucleic acid encoding SAgs. These posttranslational modifiable sites include, but are not limited to, a C-terminal GPI anchor, glycosylation site, palmitoylation site, myristoylation or prenylation site, N-methylation site, hydroxylation site, phosphorylation site, sulfation site, signal peptidase site, carboxylation site and prenylation sites.

The incorporation of many membrane proteins into the lipid environment is based on sequences of largely hydrophobic amino acids that can form membrane spanning domains. However, a large number of membrane associated proteins do not display hydrophobic elements in their primary sequences. The capacity for membrane association in these cases is often provided by covalent attachment (either cotranslationally or post translationally) of lipid groups to the polypeptide chain. Acylation of proteins by addition of C14 myristic acid to an N-terminal gly residue or addition of C16 palmitic acid by thioester linkage to cysteine residues is in a variety of positions in SAgs. Paimitoylation of SAgs is not restricted to thioester linkage and may occur through oxyester linkages to serine and threonine residues. Furthermore, thioester linkage of fatty acyl groups to proteins is not restricted to palmitate. Longer chain fatty acids such as stearic acid (C18) and arachidonic acid (C20) are also produced. The addition of palmitoyl and/or myristoyl groups with varying lengths confers additional and sufficient binding energy for hydrophobic binding of proteins to receptors, membranes or lipid bilayers. The attachment of palmitate is sufficient whereas the attachment of myristate is insufficient in isolation. Palmitoylation thus provides a means for membrane anchorage of SAgs and can allow effective concentration of an enzyme or other regulatory proteins at the membrane.

Glycosylated SAg is better capable of binding to oligosaccharide receptors on blood vessels, inflammatory cells or immunocytes. Signal peptide sequences permit the SAg to be routed to various cell surface receptors. Prenylation is important in the membrane attachment and protein-protein interactions of SAgs and oncogene activation. Prenylation, or post translational enzymatic addition of prenyl, geranyl, farnesyl or geranylgeranyl, involves reactions of a prenyl diphosphate with a cysteinyl sulfhydryl group near the C terminus of the protein to give a prenyl-S-Cys moiety. Characteristically the Cys-ali-ali-Xaa sequence (“ali” is an aliphatic amino acid; Xaa is any amino acid) is recognized by the transferase that catalyzes the reaction. When Xaa is serine, alanine or methionine, the protein is farnesylated; when Xaa is leucine, it is geranylgeranylated. Farnesylation of the protooncogene p21^{ras} is integral both for its membrane association and transforming activity. Farnesylated proteins mediate the induction by IL-1b of NOS whereas a geranylgeranylated proteins repress this induction.

Nucleic acids encoding HSPs, along with their promoters, are fused in-frame (or cotransfected) with SAg nucleic acid. These include but are not limited to two recently discovered HSP genes, orf37 and orf 35 in Staphylococcus aureus that are upstream and downstream of grpE(hsp20), dnaK(hsp70) and dnaJ(hsp40) in the following sequence: orf37—hsp20—hsp70—hsp40—orf35. The promoters are located upstream of orf37 and upstream of hsp40. These fused proteins are useful as preventative or therapeutic antitumor vaccines according to Examples 15, 16, 18-23. They are also useful ex vivo for producing a population of anti-tumor T cells, NKT cells or NK cells for adoptive immunotherapy of cancer (Examples 2-5, 7, 15, 16, 18-23).

Most eukaryotic cells are decorated with chemical groups such as phosphates, methyls, sugars, or lipids during or after their translation from mRNA. These extra groups have various functions, often serving as switches or localization signals. One lipid modification is protein prenylation in which a 15-carbon farnesyl or 20-carbon geranylgeranyl group is attached to the protein's —COOH terminus followed by other modifications (proteolysis, methylation, and palmitoylation). Most prenylated proteins are members of signal transduction cascades. For example, the -subunits of heterotrimeric guanosine triphosphate (GTP)-binding proteins (G proteins) and virtually all members of the Ras superfamily of proteins. Farnesylation of H, K, N-Ras is essential for the ability of oncogenic mutants of these proteins to transform cells. 30% of established tumor cell lines contain mutationally activated Ras proteins. FTase inhibitors shrink tumors in animals to an undetectable size with no significant toxicity after weeks or months of exposure. Farnesylation is a prerequisite for palmitoylation. Palmitoylation of H-Ras occurs only in the plasma membrane by a putative membrane-bound palmitoyl transferase. Farnesylation may bring a finite amount of H-Ras to all cell membranes, at which point and palmitoylation is required to trap it in the plasma membrane. H-Ras palmitoylation like G protein-subunit palmitoylation, is reversible and may regulate signal transduction. COOH terminal proteolysis of prenylated proteins and methylation are required for palmitoylation, membrane binding and Ras function. Prenyl protein specific protease and methyltransferase like Ftase may be good targets for drugs that prevent oncogenesis.

Common N terminal additions are fatty acid acylations and glycosylations which provide polypeptide chains with short “lipophilic handles” or recognition sites that serve to facilitate their vectoral transport or compartmentalization are common N-terminal additions. For example, myristic acid in the form of myristyl CoA serves as a substrate for specific N-terminal acylations that are important in anchoring proteins to endoplasmic membranes. The most common C-terminal modifications are amidations, acylations, polyadenylations and the enzymatic additions of tyrosyl residues. Similarly the C-terminal acylation process is complex. Prenylation occurs at Cys residues is often associated with proteins that end in (SEQ ID NO: 163) Cys-Val-Ile-Ala. The reaction sequence involves (1) a first prenylation (addition of a farnesyl moiety to Cys) followed by (2) cleavage of the Ala, Ile and Val residues and (3) the carboxymethylation of the resulting C-terminal prenylated cysteine. In addition to providing a membrane anchor, this modification often is essential to function of oncogenes such as Ras.

Two separate and well characterized pathways for carbohydrate addition: the N-linked dolichol pyrophosphate mediated pathways and the O-linked pathways that utilize UDP sugars as substrates and hydroxylated amino acid side chains as sites for attachments. Side chains amino phosphorylation of specified proteins usually at tyrosyl or serinyl residues as a way of causing cascade-like amplifications in a metabolic system. Methylation and methyl additions can also serve as novel on-off switches for metabolic processes. The targeted amino acids or methyl additions are lysine, histidine and arginine. In prokaryotes, reversible methylations of aspartyl and glutamyl side chains can occur. The best example is carboxymethylation of glutamate which is associated with bacterial chemotaxis and is elaborated by the opening and closing of membrane ion channels upon methylation and demethylation. Post translational modifications can lead to crosslinking and stabilization of protein matrices. Amino acids such as L-lysine, L-glutamine, L-cysteine and L-tyrosine are utilized extensively as sources for protein cross-linking. Examples include the extracellular matrix cross linking of collagen and elastin and the stabilization of keratin-derived matrices and tubulin by -glutamyl lysine crosslinks.

In bacteria the majority of proteins that form durable wall associations possess either distinctive N-terminal signals (lipoproteins) or more commonly distinctive C terminal wall associating signals although a number of wall associated proteins possess neither of these types of signals. A number of wall-associated proteins in gram-positive bacteria are anchored to the external surface of the cytoplasmic membrane via a covalently attached lipid moiety. Both gram-negative and gram-positive lipoproteins possess similar distinctive N-terminal signal sequences which contain a tetrapeptide consensus at the cleavage site consisting of Leu-X—Y-Cys where X and Y are predominantly small neutral residues and signal and signal peptidase cleavage occurs between Y and Cys. This sequence directs either co- or post translational modifications involving transfer of glycerol from phosphatidylglycerol to the +1 Cys, followed by the transfer of fatty acids from phospholipid to the glyceryl-prelipoprotein to produce a diglyceride-prelipoprotein. The C terminal end of a large number of Gram positive wall-associated proteins share common structural features that are required to localize these proteins in the cells wall. These C-terminal structures include a number of distinct features. At the extreme C-terminus there is a stretch of 15-22 hydrophobic residues, followed by a short tail of e predominantly charged amino acids. Immediately upstream from this hydrophobic/charged-tail domain, there is a highly conserved (SEQ ID NO: 164) Leu-Pro-X-Thr-Gly-X (LPXTGX) motif which is usually preceded by a sequence containing a high proportion of regularly spaced prolines. GPI anchors have not been identified on bacterial cell surface proteins. But the strong conservation of the SEQ ID NO: 164 LPXTGX motifs and of a hydrophobic/charged tail residue-helical domain are common structural features that are required to localize these proteins in the cell. Protein A is covalently coupled to the cell wall whereas of the proteins are not. Non-covalent interactions may occur in some proteins holding it in the cell wall while cross-linking occurs around proline rich region to form peptidoglycans. Hydrogen or water binding sites can be created by hydroxylation reactions, e.g., hydroxylation of proline in collagen provides sites for intrachain hydrogen and H2O bonding.

SAg-encoding nucleic acid is transfected into cells together with coding regions to permit the above post translational modifications which contribute to the production of an immunogenic tumor cell, accessory cell (preferably a DC) or a tumor cell/accessory cell hybrid. Such nucleic acids encoding the sites for post-translational modifications of SAgs are useful in the structural modification, translocation, cell surface binding and association with key energy-producing and signal transduction molecules and receptors. The cells expressing the products of these post-translational modifications arc useful as a preventative or therapeutic anti-tumor vaccine according to Examples 15, 16, 18-23). They are also useful ex vivo for producing a population of anti-tumor T cells, NKT cells or NK cell for adoptive immunotherapy of cancer (Examples 2-5, 7, 15, 16, 18-23).

38.SAgs and SAg Proteomes for Enhanced Immunogenicity, Specificity and Intracellular Trafficking of Soluble or Cell-Bound Binary or Ternary Complexes

SAgs with genetically engineered binding sites are provided in order to enhance their coupling to bioreactive complexes, peptides and LPS's and galactosylceramides. SAgs with a glycosylation other glycosylceramide binding site bind to glycosylceramide-CD1 or glycosylceramide-CD1 complexes alone in soluble or immobilized form, or cell bound after binding to a receptor on a T cell or NKT cell. SAgs are also provided with an LPS binding site for binding to soluble, immobilized or cell bound LPS-CD14 complexes.

SAgs are provided with a glycosphingolipid or glycosylceramide site by which they can bind to CD1-glycosylceramide or CD1-glycosphingolipid complexes present in soluble, immobilized form or affixed to CD1+ cells or NKT cells. Glycosylated SAgs are bound to CD1-glycosylceramide complexes in soluble form or fixed to CD1+ cells or NKT cells. SAgs are also provided with an overexpressed site for MHC class I molecules, to increase the effectiveness of binding to MHC class I-tumor peptide antigen complexes or TCR-bound MHC class I-tumor peptide complexes.

SAgs are engineered with repeating peptides which bind to the Vb chain to increase clustering. SAgs with an “overexpressed” (in terms of number) SAg receptor site binds to tumor cells expressing SAg receptors. SAgs possess a site for binding HSPs which are useful in immunizing normal or anergic T cells in a tumor patient. SAgs bind to T cell antagonist MHC-tumor peptide complexes converting the binary complex to a ternary complex with T cell agonist activity. Anergic T cells are activated by these ternary complexes.

SAgs are prepared with an overexpressed site for binding glycosphingolipids or glycosylceramides. These complexes are loaded onto CD1 receptors of antigen presenting cells and presented to the tumor bearing host either in vivo or ex vivo (Examples 4, 5, 7). SAgs with a myristoylation site will bind to bacterial glycolipids such as lipoarabinan or a mycolic acids The binary complex is then loaded onto APCs expressing CD1 receptors. These cells are then used in vivo (Example 14, 15, 16, 18-23) to produce a tumoricidal response. Alternatively, they are used ex vivo to produce tumor specific effector T or NKT cells for adoptive immunotherapy (Examples 2, 7, 14, 15, 16, 18-23).

A SAgs may also be prepared with signal sequences for protein sorting and intracellular trafficking. Signal sequences comprise short stretches of amino acids located at the N terminus of a protein, the C terminus or in the middle of the peptide chain. The physical properties of these sequences e.g., their polarity or charge. Signal regions are three dimensional domains on the surface of a protein made up of different fragments of the same peptide chain or by different chains altogether. Structural signals are recognized and bound by receptors located on the membranes of organelles. Signal sequences also serve as recognition sites for enzymes which modify the proteins altering their properties and bring about a change in their fate. Once they have fulfilled their function, some of the signal sequences are removed by sequence specific hydrolases. Signal peptides fused to SAgs guide them to the secretory or exocytosis pathway, or to proteins localized to the endoplasmic reticulum, lysosomes, mitochondria, nucleus, peroxisomes or secretory vesicles.

GPI-SAg-Ceramide or GPI-SAg-CD1-Ceramide Complexes Expressed on Tumor Cells, Antigen Presenting Cells, Yeast Displays, Sec Yeast Mutants, APC/tc Hybrid and Shed as Exosomes

Cells expressing, overexpressing or shedding GPI proteins are prepared so that they comprise covalently- or noncovalently bound mono- or diglycosylceramides with terminal or subterminal α1-2, α1-4 or α1-6 configurations and SAg protein or peptide moieties.

The synthetic pathway involves transfection of SAg DNA into a tumor cell or accessory cell or a hybrid thereof. The SAg protein is translated in a precursor form consisting of a receptor-coding region sandwiched between amino and carboxy-terminal sequence signals. In the endoplasmic reticulum, the signal peptides are cleaved and a GPI anchor comprising a glycosylceramide optionally bonded to a phytosphingosine chain is attached at a specific site designated w. Further post-translational modifications are made in the Golgi before trafficking to the outer leaflet of the plasma membrane. Once GPI-SAg molecules arrives at the cell surface, they may remain entirely mobile within the lipid bilayer or may associate within membrane subdomains.

GPI-SAgs are released from the cell surface into the extracellular milieu. They leave the cell surface as SAg-glycan-lipid complexes, as SAg-glycan complexes or as free SAgs devoid of a GPI anchor. GPI-SAgs released from intact cell are also released free of their lipid moiety, hence their designation as LIP(−) GPI-SAgs, whereas those presumably released with an intact lipid moiety are termed LIP(+) GPI-SAgs. The lipid free moieties are more hydrophilic and therefore soluble in an aqueous environment, whereas the intact lipid-glycan-protein complexes travel in more hydrophobic environments. In the absence of detergents, the released or “shed” LIP(+)-GPI-SAgs in vivo are vesicles with clearly defined lipid bilayers or as hydrophobic aggregates lacking a bilayer morphology. These shed vesicles, often referred to as exosomes, contain many LIP(+) GPI-SAgs. The shedding process itself appears to depend on GPI-proteins, because vesiculation is reduced by 50-90% in cells lacking GPI proteins.

Shedding is enhanced by treating the tumor cells with 20 mM retinoic acid. In addition high concentrations of glycosphingolipids on the tumor cell surface are generated by selective transport from the site of synthesis to the cell surface. Provision of ceramide containing the a2-hydroxy fatty acid C6OH results in (1) conversion to galactosylceramide, galabiosylceramide and sulfatide and (2) sorting in the trans-Golgi network to the tumor cell surface. GPI-SAgs remain biologically active after being released from the outer leaflet of cell membranes. LIP(+) GPI proteins may also transit to adjacent membranes where they associate with the exogenous membranes by incorporating themselves into the lipid bilayer in addition to binding to surface receptors.

Additionally, superantigen or oxyLDL receptor nucleic acids are transfected into yeast sec mutant. The yeast sec mutant,6-4, contains a temperature senstive mutation in a gene product required for the transport of secetory vesicles for the trans-Golgi network to the plasma membrane. Gene expression is initiated by an inducible promoter concomitant results in the arrest of vesicle fusion and the insertion of SAg or LDL receptor protein in the plasma membrane. Thus gene expression begins at the same time that secretory vesicles become unable to fuse with the plasma membrane, ensuring that the desired gene products accumulate in the membranes of these vesicles. The purification of these vesicles is rapid and simple, thereby facilitating the subsequent characterization of the desired gene product. Because the Sec6 protein is known to be involved only in the fusion of these vesicles with the plasma membrane, translocation and processing of proteins in the endoplasmic reticulum and processing in the Golgi are largey unaffected by the Sec6 mutation. The transfected superantigen or LDL nucleic acid (plasmid) is expressed as superantigen polypeptide or oxyLDL receptor polypeptide in vesicles in association with yeast GPI-lipid membrane structures. The lipid portion of the SAg-GPI-lipid complex comprises a ceramide with a C26 dihydroxy sphingosine or phytosphingosine configuration which is essential for activating NKT cells. The resulting SAg-GPI-phytosphingosine vesicles have the capacity to activate T cells via the superantigen and NKT cells via the phytosphingosine and thus produce a potent anti-tumor effect. Administered preferably by direct administration into the tumor the oxyLDL receptors induce an excessive accumuation of endogenous or exogenously administered oxyLDL and LDL at the tumor site. The deposited oxyLDL induces apoptosis and foam cell formation in tumor cells and tumor microvascular enodthelial cells resulting in potent tumoricidal response. Optionally, SAg-GPI-phytosphingosine are expressed on these vesicles together with vesicles expressing oxyLDL receptor-GPI-phytosphingosine or oxy LDL receptors. Tumor associated peptides and polypeptides, tumor apoptosis inducing peptides and polypeptides including but not limited to thrombospondin, angiogenesis inhibitor peptides and polypeptides including but not limited to angiostatin or VEGF are also useful fused or conjugated to SAgs in the same sec mutant or coadministered with the SAg-sec mutant in a separate sec mutant. Vesicles containing all of the above constructs including but not limited to SAg-GPI-phytosphingosine or oxyLDL receptor-GPI-phytosphingosine are prepared and isolated according the method of Coury L A et al, Methods in Enzymology 306: 169-186 (1999) and as in Examples 4, 5, 7, 42, 50-51.

All of the constructs given above are administerd preferentially by direct intratumoral injection as given in Example 20. They are useful in vivo as a preventative or therapeutic antitumor vaccine according to Examples 2, 7 14-16, 18-23, 36. They are also useful ex vivo to produce a population of T or NKT cells for adoptive immunotherapy of cancer (Examples 2-5, 7, 15, 16, 18-23).

A yeast cell display system is also used to present SAg to T cells, NKT cells or NK cells in the context of phytosphingosine groups present in the yeast cell membrane. The yeast displays are prepared by the method of (Cho B K et al., J. Immunologic Methods, 220: 179-188 (1998) and used to acitvate T cells, NKcells and NKT cells. The yeast presentation has the advantage of presenting as many as 10⁵ proteins per cell at the yeast surface potentially allowing multivalent interactions to occur between the yeast and target cell. The superantigen gene is transfected into the S. cervevisiae. Individual colonies of the transfected yeast are grown overnight in the Trp-media and harvested in the log phase. Murine splenocytes (10⁵) are incubated with varying number of yeast cells that bear the superantigen. After 20 h in culture, cells are harvested, washed in PBS and screened for activation markers. In this yeast display system constructs including but not limited to SAg-phytosphingosine or SAg-GPI-phytosphingosine, SAg-lipid or lipoprotein conjugates are presented to T cells and NKT cells to produce a population of T cells, NKT cells or NK cells useful for the adoptive therapy of cancer under protocols given in Example 2, 7, 14, 15, 16, 18-23. They are also useful as a vaccine or against established tumor as given in Examples 14, 15, 16, 18-23.

39.Effector T Cells: Methods of Lowering Activation Threshold for Activation by SAg

Tumor peptide MHC complexes are insufficient to activate T or NKT and may even induce antagonism or anergy. SAgs added to the complexes are useful to overcome activating T or NKT cells and overcoming the anergy common in tumor-bearing hosts. To enhance responsiveness to tumor-peptide-MHC-SAg complexes and to overcome anergy, it is desirable to reduce the threshold for signal transduction in an effector T or NKT cell population. To accomplish this, nucleic acids encoding SAg-specific TCR Vβ regions are transfected into T or NKT cells to duplicate or otherwise induce overexpression. In addition, measures are taken to alter signal transduction by dimerizing the tyrosine kinase receptors or deleting the inhibitory region of the TCR.

Most SAgs show selective binding to well defined segments of the Vβ chain of the TCR. The TCR genes are clustered on chromosome 7 and include 75-100 V, 2D, 13 J, and 2 Cβ genes. The entire 685-kb human locus has been sequenced, the longest contiguous subfamilies that exhibit >75% sequence identity at the DNA level. The human TCR locus is on chromosome 14 and consists of 42 V genes, 61 J genes and 1 Cβ gene. The TCR β chain genes are on chromosome 6 and consist of approximately 23V, 2D, 12J, and 2C gene segments. The 2 Cβ genes form clusters with upstream Dβ and Jβ segments: Cβ1 rearranges only with Dβ1/Jβ1 genes whereas Cbβ2 rearranges with both Dβ and Jβ segments. Similarly, functional Vβ genes appear to rearrange to both J clusters in a random fashion. The b chain transcripts of antigen-specific T cell clones appear to contain little length variation and harbor conserved N additions.

A mechanism for achieving diversity in variable (antigen-specific) regions of the TCR involves the random addition of nucleotides inserted at junctional positions during the joining of VβDβJ segments. It is at this position that nucleic acids encoding the major Vb binding site for a specific SAgs are inserted. This overexpression allows for more selective recognition of SAg and a lower activation threshold by a SAg that selectively binds at that site.

Nucleic acid encoding SAg receptor is amplified and transfected into T cells to overexpress the SAg receptor on the cell surface These T cells bind SAgs, and this is linked to appropriate signal transduction pathways that deliver a mitogenic signal to the T cell. One method of increasing T or NKT cell reactivity to a SAg is to increase the density of their SAg receptors. Even in the absence of ligand, the equilibrium is shifted from monomeric inactive receptors to dimeric or oligomeric active receptors. Concomitant expression of the corresponding ligand reinforces the signal. Increased numbers of receptors occur after increased transcriptional activation of, or amplification of, the SAg receptor gene. Amplification is the preferred method.

The SAg receptor may also be mutated so that it engages in ligand-independent dimerization. Examples of such mutations are addition or loss of a cysteine residue in the extracellular domain causing formation of dimeric and disulfide bonded and activated receptors. In addition it is possible to dimerize tyrosine kinases by fusing a tyrosine kinase catalytic domain to a protein which is a functional dimer. These fusion partners are able to form homodimers. Such a fusion protein results in dimerization of kinase domains which allows their autophosphorylation and activation. Interaction with receptors in a manner which promotes dimerization of two different receptors is another method to enhance receptor reactivity. The kinase domain of a receptor may be mutated to increase catalytic activity or alter substrate specificity. Such mutations expand quantitatively and qualitatively the repertoires of substrates in the target cells and thereby shift the balance towards activation and transformation. Mutations in regions involved in negative regulation of receptor function also contribute to the transforming properties. Loss of regions in the C terminus that are regulatory serine phosphorylation or autophosphorylation sites also contributes to excessive receptor activity.

Effector cells as discussed above are prepared as in Examples 4, 5, 7. They may also be used in vivo as tumor specific effector (T or NKT) cells for the adoptive immunotherapy of cancer (Examples 2-5, 7, 15, 16, 18-23).

40.SAg Nucleic Acids Fused of Cotransfected into Tumor Cell with Nucleic Acids Encoding Inducible Nitric Oxide Synthase (iNOS)

SAg-encoding nucleic acid is fused in frame (or cotransfected) with nucleic acid encoding inducible nitric oxide synthase which produces nitric oxide (NO). NO is derived from terminal guanido-nitrogen of L-arginine which is catalyzed by the constitutive or inducible nitric oxide synthase (iNOS). NO is pleiotropic and is a major cytotoxic mediator secreted by activated endothelial cells and macrophages. Production of NO is associated with apoptosis of tumorigenic cells and with a bystander effect on surrounding non-NO producing tumor cells (bystander effect). Non metastatic tumor cells show high levels of iNOS activity and NO, whereas metastatic cells do not. There is an inverse relation between production of endogenous NO and the tumor cells survivability. In the present invention, tumor cells transfected with SAg-encoding nucleic acid are cotransfected with nucleic acids encoding iNOS. The gene for iNOS has been cloned and characterized by Xie Q et al., Science 256: 225-228 (1992). Tumor cells cotransfected with nucleic acids encoding SAgs and iNOS demonstrate augmented immunogenicity via the expression of SAg as well as enhanced auto- and bystander tumoricidal capacity via NO production.

After administration to a patient and colonization of metastatic sites, the transfectants induce a powerful local and systemic tumoricidal effect. The presence of NO allows the transfectants to die naturally via auto-apoptosis within a finite period (usually 72 hours) after administration thus minimizing the risk of inducing active metastatic disease. These tumor cell transfectants may also be made to express oncogenes associated with the metastatic phenotype to promote localization of the cells to tumor sites in vivo. The cells may be further transformed by nucleic acid encoding angiostatin or other angiogenesis inhibitors for additional tumoricidal potency. The transfectant are prepared by methods in Example 1-3 and used as a preventative or therapeutic antitumor vaccine by methods in Example 15, 16, 18-23).

41.DCs, Other Accessory Cells and DC/tc Hybrids Expressing and/or Secreting SAg

Accessory' cells are necessary to generate primary antibody responses in culture. Of the various types of accessory cells, DCs are the most effective APC. DCs are a preferred accessory cell. However, the invention is not confined to DCs. Any other accessory cell type may be used in place of DCs. In particular, accessory cells are defined in Oxford's Dictionary of Biochemistry and Molecular Biology as including fibroblasts, synoviocytes, macrophages, B cells, Langerhans cells and any other cell type which assists in producing an immune response of any kind.

DCs have exceptional capability to capture antigens, process and present antigenic peptides, migrate to lymphoid organs, and induce primary immune responses of both CD8+ and CD4+ T cells. The ability of DCs to act as potent APC in the induction of T cell responses is attributed to the high expression of MHC molecules and adhesion and/or costimulatory molecules as well as the cells' capacity for to producing cytokines essential for the activation and proliferation of the T cells.

The number of molecules of antigen-MHC complex on tumor (and infected) cells is typically small (100 per cell), and are recognized by rare T-cell clones (at a frequency 1/100,000) via a TCR that has a low affinity (1 M). In vitro or in vivo, only a few DCs are necessary to provoke a strong T-cell response. In the mixed leukocyte reaction, one DC was sufficient to stimulate 100-3,000 T cells. MHC products and MHC-peptide complexes are 10-100 times higher on DCs than on other APCs such as B cells and monocytes. Mature DCs resist the suppressive effect of IL-10, but synthesize high levels of IL-12 that enhances both innate (NK cell) and acquired (B and T cell) immunity. DCs also express many accessory molecules that interact with various molecules or receptors on T cells to enhance adhesion and signalling (co-stimulation): examples of such pairs are LFA-3/CD58, ICAM-1/CD54, B7-2/CD86. Tumor cells that express the B7 gene elicit CTLs against otherwise silent, subdominant tumor antigens. All these properties of DCs (MHC expression, CD1 expression, secretion of IL-12 and the expression of co-stimulatory molecules) are upregulated within a day of exposure to many stresses and “dangers” including microbial products.

Infected cells and tumors frequently lack the costimulatory molecules that drive clonal expansion of T cells, the production of cytokines, and T cell development into killer cells. Located in most tissues, DCs overcome challenges by capturing and processing antigens, and displaying large amounts of MHC-peptide complexes on their surface. They upregulate their co-stimulatory molecules and migrate to lymphoid organs, the spleen and draining lymph nodes, where they activate antigen-specific T cells. AU of these activities of DCs can be induced by infectious agents and inflammatory products, so that DCs appear to function as “mobile sentinels” that not only bring antigens to T cells but also stimulate those T cells in the induction of immunity.

DCs are present in most tissues in a so-called “immature” state, unable to stimulate T cells. Although these DCs lack the requisite accessory signals for T-cell activation, such as CD40, CD54 and CD86, they are well equipped to capture antigens, a key event in the induction of immunity; the antigen is then able to induce full maturation and mobilization of the DCs. Terminally-differentiated or mature DCs can readily prime T cells Once activated by DCs, these T cells can complete the immune response by interacting with B cells for antibody formation, macrophages for cytokine release, and target cells resulting in lysis. Thus, immature DCs first handle antigens and then, as mature DCs a day or more later, they potently stimulate T cells.

DCs stimulate CTLs, which express the accessory molecule CD8 and interact with MHC class I bearing cells, to proliferate vigorously. In the presence of mature DCs and of IL-12, CD4-expressing T-helper cells turn into interferon gamma (IFN)-producing TH-1 cells. IFN activates the antimicrobial activities of macrophages and, together with IL-12, promotes the differentiation of T cells into killer cells (CTL). The capacity of DCs to produce IL-12 and stimulate TH-1 cells leads to microbial resistance. Through IL-4, DCs induce T cells to differentiate into TH-2 cells which secrete IL-5 and IL-4, activate eosinophils and help B cells generate an antibody response, respectively. DCs respond to T cells as well. CD40 and the newly described TRANCE/RANK receptor on DCs are ligated by the TNF (tumor-necrosis factor) family of proteins expressed on activated and memory T cells; this leads to increased DC survival and, in the case of CD40, upregulation of CD80 and CD86, secretion of IL-2 and release of chemokines such as IL-8 and MIP-la and b

Immature DCs capture antigen (and particles and microbes in general) by phagocytosis. They then form large pinocytic vesicles in which extracellular fluid and solutes are sampled, a process called macropinocytosis. Finally, they express receptors that mediate adsorptive endocytosis, including C-type lectin receptors like the macrophage mannose receptor and DEC-205, as well as Fc, located in most tissues, and Fc receptors. Macropinocytosis and receptor-mediated antigen uptake make antigen presentation so efficient that picomolar and nanomolar concentrations of antigen suffice, much less than the micromolar levels typically employed by other APCs. However, once the DC has captured an antigen, which also provides signals to mature, its ability to capture antigens rapidly declines, and the cell begins to assemble antigen-MHC class II complexes.

An antigen enters the endocytic pathway of the DC. DCs produce large amounts of MHC class II-peptide complexes due to specialized, MHC class II-rich compartments (MIICs) that abound in immature DCs. MIICs are late-endosomal structures that contain the HLA-DM or H-2M products, which enhance and perform editing functions in the binding of peptide to MHC class II. During maturation of DCs, MIICs convert to non-lysosomal vesicles that discharge their MHC peptide complexes to the surface.

To generate cytotoxic killer cells, able to eliminate infected cells, and attack tumor cells and transplanted foreign cells, DCs must present peptides (complexed generally to MHC class I proteins) to CD8+ T cells. Display of peptide-loaded MHC class I complexes on e the DC surface follows translocation by a peptide transporter from the cytosol to the ER, where complexing occurs and then to the surface.

Human DCs are characterized by a pattern of surface markers and have the phenotype CD1a+, CD3^{neg}, CD4^{neg}, CD8^{neg} CD20^{neg}, CD40+ CD86+ in the human. The murine phenotype is and CD3^{neg} CD4^{neg}, CD28^{neg}, CD8-B220^{neg}, CD40+, CD80+ and CD36+.

Maturation of DCs is required for the initiation of an immune response. Microbial products including whole bacteria and the bacterial cell-wall component LPS and inflammatory mediators such as IL-1, GM-CSF and TNF, stimulate DC maturation, whereas IL10 blocks it. Ceramide, which is induced by maturation signals, shuts down antigen uptake by the DC. Mature DCs express high levels of the NFkB family of transcriptional control proteins (RelA/p65, RelB, RelC, p50, p52) which regulate the expression of many gene encoding immune and inflammatory proteins. Signalling through the TNF-receptor family, for example TNF-R (CD-120a/b), CD40, and TRANCE/RANK, results in activation of NFkB. Therefore, to induce an immune response through activation of DCs, a pathogen or antigen may have to mobilize the signal transduction pathways of the TNF-R family and TNF-R-associated factors (TRAFs).

One explanation for the failure of the immune system to eradicate most immunogenic tumors is the lack of tumor antigen presentation by DCs in vivo. Several strategies using tumor antigen-charged DCs as vaccines for cancer immunotherapy have been developed. Immunization with DCs pulsed with purified tumor-associated peptides or proteins has been shown to be a powerful method of priming tumor-reactive T cells and inducing host protective and therapeutic antitumor immunity in mice and man. However, such a clinical approach is currently limited due to the paucity of identified human tumor rejection antigens. The polymorphism of the HLA system has also made it difficult to identify tumor-associated peptides as cancer vaccines. In human melanoma, a class of tumor-associated proteins has been identified. However, it is unclear which antigen is the best choice for effective tumor rejection in vivo or how effective any such antigen may be. Thus, immunization with defined tumor antigens is currently limited to a small number of cancers in which candidate antigens have been identified. Anichini et al, J. Immunol. 156:208-217 (1996), showed that the majority of CTL present in HLA-A2.1+ melanoma patients were not directed to the known tumor antigens, Melan-A/Mart-1, tyrosinase, gp100 or MAGE-3. Therefore, immunization with other, yet unidentified, antigens would be more effective in eliciting tumor immunity in these patients. Johnston et al., J. Exp. Med. 183:791-800 (1996) demonstrated that the enhanced immunogenicity of tumor cells engineered to express the B7-1 gene was a result of expansion of the antigenic repertoire of the tumor. This implies that vaccination with multiple tumor antigens may be superior to use of a single dominant epitope. Indeed, in situations where a tumor-associated antigen remains unidentified, a novel approach is needed for presentation of that antigen by a professional APC.

An alternative approach, not encumbered by these limitations, is to use unfractionated tumor peptides or tumor proteins as a source of tumor antigens. Two studies have shown that administration to mice of APC (from the spleen) or epidermal Langerhans cells pulsed with tumor fragments resulted in protective immunity against tumor challenge. Zitvogel et al., J. Exp. Med. 183:87-97 (1996) showed that vaccination of mice with bone marrow-derived DC pulsed with unfractionated tumor peptides reduced the growth of subcutaneously established, weakly immunogenic tumors. Thus, immunization with multiple tumor antigens may be superior to use of a single dominant epitope.

One approach to overcome the possible drawbacks of unfractionated tumor antigens is to use mRNA from tumor cells as a “source” of antigen. mRNA can be amplified from a very small number of cells, permitting the generation of sufficient amounts of antigen from minute amounts of tumor tissue Moreover, tumor-specific mRNA can be enriched by subtractive hybridization to remove RNA that is common to normal tissue. This increases the levels of the relevant tumor-specific antigen(s) that can be achieved, and hence, the potency of the vaccine. More importantly, this approach reduces the concentration of nonspecific antigens or, possibly, self-antigens, thereby lessening the potential for autoimmunity. Pulsing DCs with RNA is known to be effective in empowering them to induce CTL responses and tumor immunity.

The fusion of tumor cells with DCs is another approach to generate a hybrid vaccine that has both potent antigen processing/presenting power along with the endogenous expression of multiple tumor antigens. Such a hybrid cell would be more effective in inducing antitumor immunity. Gong et al., Proc. Natl. Acad. Sci USA 26:6279-6283 (1998), demonstrated that fusion of a relatively immunogenic mouse tumor, MC38 carcinoma, with syngeneic DCs resulted in a vaccine that induced (1) T cell protective immunity against tumor challenge and (2) rejection of an established tumor. Wang et al., J. Immunol. 161:5516-5524 (1998) used the poorly immunogenic B16 (B16.F10) melanoma which does not express MHC and costimulatory molecules. Immunization with irradiated B16 tumor cells failed to induce systemic immunity or elicit functional tumor-reactive T cells. RMA-S is a Rauscher MuLV-induced T cell lymphoma originating in a C57BL/6 (“B6”) mouse that is genetically defective in TAP, and thus, does not process endogenous antigens for binding to MHC. Fusion of DCs with syngeneic tumor cells generated hybrid cells that expressed both DC-associated accessory molecules important for antigen presentation and tumor-derived antigens. The DC/tc hybrids were processed and presented tumor-associated antigens and elicited tumor-reactive CTLs. Vaccination of B6 mice with B16/DC hybrid cells induced partial protective immunity against tumor challenge. Immunization with B 1 6/DC or RMA-S/DC hybrid cell vaccines primed lymph node (LN) T cells, which, after expansion ex vivo, were active in adoptive immunotherapy. The transfer of such vaccine-primed, expanded T cells into tumor-bearing mice reduced the number of established B16 pulmonary metastases and, in the case of RMA-S/DC, effectively eradicated disseminated FBL-3 tumor.

The present invention includes a hybrid cell made from fusion of a tumor cell and a DC cell further transformed or transfected with a SAg. Nucleic acids encoding SAgs may be introduced into either the tumor cells or the DCs prior to fusion as in Example 1, 2, 3, 25, 26. This fused cells are prepared as in Example 24, 25 and their phenotype established by the retention of DC characteristics, tumor cell antigens and the expression of SAg (Example 25). By virtue of these multiple features, this SAg-expressing DC/tc has the unique capacity activate maximally an anti-tumor immune response.

SAg stimulation is known to activate CD4+ and CD8+ T cells to recognize and lyse tumors specifically both in vitro and in vivo. The DC component of the hybrid cell provides optimal tumor antigen presentation due to its enormous surface area together with natural expression of costimulatory molecules B7.1, B7,2, adhesion molecules ICAM-1 and ICAM-3, MHC class I and class II and CD1 receptors. B7.1, in particular, provides a basis for expanding the epitope recognition spectrum from dominant to subdominant epitopes. The expressed SAg confers upon the hybrid cell an augmented capacity to activate various classes of cells that mediate both innate and “acquired” or adaptive immunity, including CD4+ and CD8+ T cells, NK cells and NKT. The SAg also contributes to generation of TH-1 cytokines by this class of T helper cells which contributes to an optimal anti-tumor response. The DC/tc hybrid that expresses and/or secretes SAg is abbreviated herein as an “S/D/t” cell and combines the potent activating properties of SAg with the specialized (tumor) antigen presenting capacity of the DC and the tumor antigens provided endogenously by the tumor cell partner. This S/D/t cell thus consolidates in a single cell the capacity to unleash and amplify the full weight of the host immune response specifically against a selected array of tumor associated antigens.

The present invention also includes the additional introduction, into the S/D/t cell of with additional nucleic acids. In one embodiment, the additional nucleic acid encodes the particular galactosyltransferase enzyme that catalyze the synthesis of the “heterograft epitope” α Gal. In another embodiment, the additional nucleic acid encodes enzymes that synthesize galactosylceramide which is the “natural” epitope recognized by the invariant chain of NKT cells.

To summarize the foregoing section, the present invention includes DCs, other accessory cells or hybrid DC/tc, each transformed to express SAgs as described in Examples 1 and 3. The transformed (or transfected) hybrid cell, the S/D/t cell, expresses (1) the major accessory molecules of DCs cells (such as CD40, CD80 and CD86, MHC class I and II and CD1); (2) tumor associated epitopes provided by the tumor cell fusion partner; and (3) SAg either membrane bound, secreted or both which activates T cells, NK cells and NKT cells to produce a specific or selective tumoricidal response.

While the tumor S/D/t cells are preferred, SAg-transfected DCs or other accessory cells are also effective in inducing antitumor responses. These are used as a preventative or therapeutic antitumor vaccine, or ex vivo to stimulate a population of T cells, NK cells or NKT cells for adoptive therapy of cancer (Examples 29).

42.DCs Expressing SAg and Tumor Associated Antigens—Production by Processing of Apoptotic Tumor Cells or Tumor Cell Lysates

DCs expressing SAg and tumor associated antigens are prepared without cell fusion (Example 28). Apoptotic, SAg transfected tumor cells are prepared by first transfecting tumor cells with SAg (Example 1) and then inducing apoptosis by irradiation or other methods well known in the art (Example 28). DCs express aᵥβ₅-binding integrins and secrete thrombospondin which ligates vitronectin expressed on the surface of the apoptotic tumor cell. DC surface CD36 binds to its natural ligand, sequestrin, also expressed on apoptotic tumor cells. The apoptotic SAg-expressing tumor cells are phagocytosed and processed by DCs under conditions described in Example 28.

In another embodiment, lysates of tumor cells optionally expressing SAg are also used as above. Tumor cells are first transformed to express SAg and then lysed (Example 28. These lysates are “fed” top DCs as in Example 28. DCs treated in this way can now present tumor associated antigens along with SAg to the immune system. Alternatively, DCs are first transformed to express SAg, and these cells are allowed to phagocytose or process apoptotic tumor cells or lysates. Optionally the tumor cells may have been previously genetically modified with nucleic acids so that they synthesize β-1,3-glucans, LPS, peptidoglycan or Gal Cer.

The resulting SAg-expressing DC, after phagocytosing apoptotic tumor cells or lysates, expresses MHC class II, costimulatory molecules CD 40, CD80 and CD86, together with SAg and tumor associated antigen. The additional expression of SAg in this system permits more potent activation of T cells, NKT cells and NK cells which recognize the tumor associated antigens expressed on the DC surface in the context of MHC and costimulatory molecules.

In another embodiment, tumor cells are fused to mammalian cells including but not limited to proximal tubular epithelial cells, other kidney cells including the Madin-Darby canine kidney (MDCK) cell line which express an abundance of alpha anomeric digalactosylceramides which are natural superantigen receptors. In an additional embodiment, tumor cells are fused to cells including but not limited to amphibian intestinal cells which express a high level of phytosphingosine (see Example 25 for cell fusion methodology). The resulting hybrid cells express tumor associated antigens and either galactosylceramides or phytosphingosine. Alternatively, exogenous galactosylceramide or phytosphingosine from the cell membranes of the above kidney or amphibian cells are incorporated into intact tumor cells by methods given in Section 38 and Example 5. These hybrid tumor cells or tumor cells with newly acquired membrane glyco- or phytolipids (TCGP) are further transfected with superantigen nucleic acids to produce hybrid tumor cells or TCGP expressing superantigens, tumor associated antigens, galactosylceramides and/or phytosphingosine (Example 1). These hybrid tumor cells or TCGP are potent activators of T cell, NK cell and NKT cells

The DCs, hybrid tumor cells, or TCGP given above are used in a preventative or therapeutic antitumor vaccine (Example 29) or ex vivo to activate T cells, NKT cells or NK cells for the adoptive immunotherapy (Example 29).

43.DCs Expressing or Secreting SAg Cotransfected with a Tumor Associated Antigen or “String of Beads” Tumor Antigens

When a dominant tumor associated antigen (protein) is known, nucleic acid encoding such an antigen are used to transform DCs which already express or secrete SAg (Example 35). Antigens identified by “SELEX” technology which consists of nucleic acids encoding tumor antigens from distinct structural and functional categories of human tumor associated antigens, including mutants, differentiation variants, splice variants, amplified/overexpressed antigens or retroviral antigens may be used. Nucleic acids encoding tumor antigens used to transfect SAg-expressing DCs or DC/tc hybrids. This invention contemplates transfecting with individual nucleic acids encoding a single antigen, or multiples as in a “string of beads” carried by adenoviral or other vectors known in the art (Example 35). Nucleic acids encoding a “string of beads” or tumor associated antigens identified by SERAX may be fused in frame (or cotransfected with) SAg-encoding nucleic acid into DCs or DC/tc. These SAg- and tumor antigen-expressing DCs or DC/tc hybrids are used as a preventative or therapeutic antitumor vaccines (Example 29) or as stimulators ex vivo of T cells, NKT cells or NK cells for adoptive immunotherapy (Example 29).

Furthermore, nucleic acids encoding proteins listed in Tables I, II, IV and V, for example, angiostatin, protein A, erb/Neu and HSPs, staphylococcal collagen adhesin, are introduced into and expressed in tumor cells or DCs that express or secrete SAg, or into S/D/t cells. These cells that coexpressing the proteins and peptides of Tables I, II, IV and V together with SAg are useful as preventative or therapeutic antitumor vaccines (Example 29) or as stimulators ex vivo that activate T cells, NKT cells or NK cells for adoptive immunotherapy (Example 29).

44.Naked DNA or RNA Obtained from the Various Cells Described Above that Express and/or Secrete SAg

DNA containing the CpG backbone is extracted from tumor cells or DCs that express/secrete SAgs or S/D/t cells (Example 30-34). The preferred source of DNA or RNA is the S/D/t cells DCs or tumor cells expressing SAg are also useful. Alternatively, the DNA or RNA can be obtained from DCs, tumor cells or DC/tc into which SAgs were introduced by the cells having phagocytosed SAg-transformed apoptotic tumor cells or tumor cell lysates.

The extracted DNA or RNA is used as a naked DNA or RNA preventative or therapeutic vaccine (Examples 30-34). Alternatively, this nucleic acid material may be used ex vivo to activate T cell, NKT cells or NK cells adoptive immunotherapy (Example 1, 31 , 33). This extracted DNA or RNA may be used in an initial step of inducing immune reactivity in regional lymph nodes of tumor bearing subjects. After this “priming,” T cells, NKT cells and/or NK cells are harvested from these lymph nodes, expanded in culture in the presence of additional SAg, SAg-expressing DC or tumor cells, or S/D/t cells to generate a T cell, NKT cell or NK cell population for adoptive immunotherapy (Examples 29). DNA or RNA for immunization may also be obtained from the various cells described above that express SAg, and which additionally express or several Staphylococcal adhesins, b-glucans, LPS, peptidoglycans, teichoic acids, mannose, mannan, protein A and/or their respective binding proteins.

Also useful for naked nucleic acid immunization are bacterial or insect nucleic acids (with CpG motifs) which encode enzymes that catalyze the biosynthesis of β-1,3-glucans, LPS, peptidoglycan, α-Gal, GalCer, teichoic acids, mannan or mannose. Also useful are bacterial or insect nucleic acids that encode the binding proteins for the above carbohydrate-based molecules, glycoprotein lectins that bind the carbohydrate structures, or protein A. Such nucleic acids are used to co-immunize along with SAg expressing DCs or tumor cells or S/D/t cells. Such combined vaccine preparations are used as a preventative or therapeutic antitumor vaccines (Examples 29, 30). Alternatively, they may be used to initiate adoptive T cell therapy by priming regional lymph nodes T cells which are harvested, expanded in vitro by stimulation with S/D/t cells, accompanied by, or followed with IL-2. The tumor antigen-sensitized T cells are reinfused into subjects as described in Example 29.

45.Exosomes Derived from (1) SAg-Expressing Tumor Cells (2) SAg Expressing-DCs (3) S/D/t Cells or (4) DC/tc Hybrid Cells

MHC-peptide complexes accumulate in endosomes and lysosomes, which compartments contain MHC class II-enriched internal vesicles that are released outside the cell following direct fusion of the external endosomal membrane with the plasma membrane. These vesicles, termed “exosomes” are capable of stimulating CD4+ T cell clones in vitro. In addition, tumor peptide-pulsed DC-derived exosomes prime specific CTLs in vivo leading to a T cell-dependent eradication or suppressed growth of established murine tumors. In the present invention, the exosomes which have SAgs in addition to tumor associated antigens and MHC class I and class II molecules are prepared. Such preparations are significantly more potent in their ability to induce shrinkage of established tumors and prevent tumor outgrowth.

Exosomes are prepared from (1) tumor cells or DCs which have been transfected with SAgs (2) S/D/t cells, (3) DCs or hybrid DC/tc which have phagocytosed SAg-expressing apoptotic tumor cells or tumor cell lysates (Example 36). In the above hybrids, either the DC or tumor cell may be transfected with SAg-encoding nucleic acid prior to fusion. The resulting exosomes express MHC class I and class II molecules, SAgs and tumor associated antigen. In order to ensure the routing of the transforming SAg to exosomes, the SAg-encoding nucleic acid should include a sorting signal to localize the SAg to the exosome. These cells may be pulsed with tumor associated antigens shortly before isolation of their exosomes. The isolated exosomes are used as preventative or therapeutic antitumor vaccines (Example 36) or as stimulators ex vivo that activate T cells, NKT cells or NK cells for adoptive immunotherapy (Example 36). These various exosome preparations are extremely effective inducers of anti-tumor responses.

46.Cell Surface Display of Recombinant SAg and Tumor Associated Antigens in Bacteria

Heterologous proteins and various carbohydrate-containing moieties, displayed on the 11 surface of bacterial cells often act as major antigenic systems that stimulate anti-tumor immunity. Such antigens include GalCer, α Gal, β1,3-glucans, LPS, peptidoglycans, teichoic acids and mannan. These structures will be referred to below collectively as “anti-tumor motifs.” These structures are created by the action of enzymes encoded by a number of bacterial and fungal genes. For example, Sphingomonas paucimobilis expresses GalCer, or Klebsiella aerobacter expresses -Gal and LPS, and Cryptococcus expresses b1,3-glucan. Because not all the genes responsible for the biosynthesis of these molecules have not been identified, it is difficult to isolate them and introduce them into mammalian cells. These structures are, however, biosynthesized in abundance by bacteria. Immunization with live recombinant bacteria induces both local and systemic immune responses suggesting that gram-positive bacteria might constitute potential live bacterial vaccine delivery systems. The surface molecules of gram-positive bacteria seem to be more permissive for the insertion of extended sequences of foreign proteins than are gram-negative bacteria, in which both translocation through the cytoplasmic membrane and correct integration into the outer membrane are required for proper surface exposure.

In the present invention, different bacterial surface display systems are used to express natural anti-tumor motifs for developing live bacterial vaccine vehicles. SAgs are provided to bacteria which do not naturally biosynthesize them so that they are expressed together with natural anti-tumor motifs made in the bacteria. These bacteria are then used as preventive or therapeutic antitumor vaccines (Example 28).

Sphingomonas paucimobilis bacteria express GalCer which can activate the Vα 14 invariant chain expressed by NKT cells. These cells recognizes the galactosylceramide epitope. NK cells, using their NKP1-1 receptors, recognize carbohydrate units such as β1,3-glucan expressed widely on fungi. NK cells are activated directly by SAgs. Further proliferation is induced by interferon produced by T cells in response to the SAg. Humans have natural antibodies specific for the aGal epitope. This epitope is constitutively expressed on several bacteria including Klebsiella aerobacter and E. coli.

Coexpression of SAg with the above anti-tumor motifs in recombinant bacteria or fungi provides potent signals to activate NKT cells, T cells and NK cells and to induce production of TH-1 cytokines. The adhesion molecule VCAM-1 expressed by some SAgs such as enterotoxin C contributes to the process by costimulation. Therefore, the SAg expressing bacteria (whether natural or transformed) are capable of activating all of the major cell types involved in the anti-tumor response.

In the present approach, the preferred SAg is SEB. SEB is introduced for surface display into S. carnosus. E. coli-staphylococcus shuttle vectors are constructed by taking advantage of (1) the promoter signal sequence and propeptide region from the lipase gene construct derived from S. hyicus and (2) the cell surface attachment part of staphylococcal protein A. A 198-amino-acid region, designated ABP (albumin binding protein), is expressed adjacent to the cell wall to increase accessibility to the surface-displayed target peptides. Staphylococcal enterotoxin B is introduced between the lipase propeptide and the ABP region and the surface exposure of the three different regions are tested separately with different assays.

These recombinant bacteria are useful as a preventative or therapeutic antitumor vaccine (Example 28) or as stimulators ex vivo that activate T cells, NKT cells or NK cells for adoptive immunotherapy (Example 28).

47.Introduction of Staphylococcal Collagen Binding Adhesins into DCs, Tumor Cells or S/D/t Cells

Nucleic acids encoding SAgs are transfected into these various cells, as described above, together with nucleic acids encoding Staphylococcal collagen adhesin. Mice immunized with a recombinant fragment of the collagen adhesin were protected against Staphylococcus aureus-mediated septic death. Sera from S. aureus-immunized mice promoted phagocytic uptake (opsonized) and enhanced intracellular killing of the bacteria compared to sera from control mice.

The collagen binding adhesin is isolated from S. aureus strain Cowan. Sequencing of the cloned corresponding gene cna revealed a 133-kDa polypeptide (close to that of 135 kDa reported for the native protein). This protein is proposed to consist of a signal sequence (S) followed by a large nonrepetitive region (A). Immediately following the A region are three consecutive repeats of a 167 amino acid long unit (B1, B2, B3). A cell wall (W) region consisting of 64 amino acid proline-and lysine-rich domain is followed by stretch of hydrophobic amino acids (M), presumably constituting the cell membrane spanning region. Finally, the C-terminus (C) is made up of a few positively charged amino acids. This model structure is used as the starting point to identify the collagen binding domain. The ligand binding site is localized within the 135-kDa S. aureus collagen adhesin. The collagen binding domain is localized to a 168 amino acid long segment [CBD (151-318)] within the N-terminal portion of the adhesin.

Using biospecific interaction analysis, bovine collagen was found to contain eight binding sites for CBD (151-318), two of which were high affinity and six low affinity. The deduced amino acid sequence of the ligand binding domain of the collagen adhesin is presented. Subsequently a discrete collagen-binding domain within the collagen adhesin was identified and localized to a region between amino acids Asp209 and Tyr233. The FDA strain 574 of S. aureus encodes a 1185 amino acid collagen adhesin. The complete nucleotide sequence of the cna gene as well as a schematic model of the collagen adhesin have been published. The overall structure resembles that of other gram positive surface structures. The lysine and proline rich hydrophilic region which follows the repeated domains resembles a structure in protein A, staphylococcal fibronectin receptor and streptococcal protein G and M proteins. Also present is the hexapeptide LPKTGM which is similar to the consensus sequence LPXTGE which is conserved among other gram positive surface proteins. The hydrophilic region is thought to mediate the binding of the protein to the cell wall. The presence of hydrophobic amino acids which may traverse the membrane followed by a C-terminal cluster of positively charged residues, possibly located on the cytoplasmic side of the membrane, is characteristic of staphylococcal cell surface proteins.

In the collagen adhesin, a 29 amino acid signal peptide at the N-terminus is followed by a large nonrepetitive A domain, and the highly homologous domains B1, B2 and B3 (probably a result of a series of stepwise gene duplication events). Collagen binding receptors have been found on other species of bacteria such as the 75×adhesin of uropathogenic E. coli. Type 3 fimbrias from pathogenic enteric bacteria, some species of oral streptococci, Streptococcus pyogenes, Yersinia and Treponema pallidium have all been reported to bind various forms of collagen. Thus the collagen binding appears to be a common modality used by pathogenic bacteria of a diverse group to adhere selectively to host tissues and form a focus of infection.

Nucleic acids encoding staphylococcus collagen adhesin are introduced into SAg-expressing tumor cells or DCs, or S/D/t cells. The cells co-expressing the staphylococcal collagen adhesin with SAgs are useful as a preventative or therapeutic antitumor vaccines (Example 28) or as stimulators ex vivo that activate T cells, NKT cells or NK cells for adoptive immunotherapy (Example 28).

48.Co-Expression of Anti-Tumor Motifs or Their Binding Proteins with SAg

Tumor cells or DCs expressing SAgs, or S/D/t cells, are transformed with nucleic acids encoding enzymes that catalyze the biosynthesis of anti-tumor motifs, including the α Gal epitope, the GalCer epitope, β-1,3-glucans, LPS, peptidoglycan, teichoic acids or a protein or peptide such as Staphylococcal adhesins, protein A, and/or the binding proteins for the above motifs or proteins. Transformation may be achieve using bacterial plasmids or nucleic acids integrated into an appropriate viral vector. These antigenic structures are fundamental units recognized in the primitive host defense mechanisms (“innate immunity”) of invertebrates, but also evoke responses in mamalian immune systems via the TOLL and NFkB systems.

DNA encoding the galactosyltransferase that synthesizes the saccharide structure containing the a α Gal epitope, and gene clusters encoding the biosynthetic pathway for LPS are described in Schnaitman C A, et al., Microbiol. Rev. 57: 655-682 (1993). DNA is extracted from bacteria which biosynthesize these molecules and used to transfect DCs, tumor cells, or S/D/t cells For creation of the GalCer structure, the source of DNA is Sphingomonas paucimobilis organisms. Nucleic acids encoding the pathways for biosynthesis of β-1,3-glucans, peptidoglycans, and protein A have been cloned from insects and Staphylococcus aureus, respectively. These nucleic acids are cloned into suitable expression vectors and introduced into the target cells. Resulting S/D/t cells thus express SAg as well as the anti-tumor motif structure.

S/D/t cells that co-express Gal can interact with and stimulate NKT cells through the Vα 14 invariant chain which naturally recognizes the -galactosylceramide epitope. NK cells, via their NKP1-1 receptors, will recognize carbohydrate units such as β-1,3-glucans on the S/D/t cells. The co-expressed SAg induces further NKT cell expansion. The SAg is also capable of inducing massive proliferation of conventional T cells which can be further promoted by the co-expression of B7-1, B7-2 and ICAM-1 which are normally expressed on DCs. VCAM-1, expressed by some SAgs such as enterotoxin C, also is capable of contributing to this stimulation. As indicated above, NK cells are activated directly or indirectly by T-cell derived interferon.

The S/D/t cells (as well as tumor cells or DCs expressing SAg) that also express one or more of the anti-tumor motifs are capable of activating all of the major cell types involved in anti-tumor immunity: T cells specific for peptides, NKT cells reactive with lipoproteins and glycosylceramides and NK cells that recognize for oligosaccharides. These cells are useful as preventative or therapeutic antitumor vaccines (Examples 29) or as stimulators ex vivo that activate T cells, NKT cells or NK cells for adoptive immunotherapy (Example 29).

49.SAgs Combined with Low Density Lipoproteins (LDL), Oxidized LDL (oxy LDL) Oxidized LDL Mimics and Apolipoproteins

In the present invention, low density lipoproteins (collectively LDL) intermediate density LDL (IDL), chylomicrons, very low density lipoproteins (VLDL), oxidized LDL (oxyLDL), oxyLDL mimics as well as and apolipoproteins including but not limited to apolipoprotein (a), B100 and E4 are conjugated to superantigens and are useful as anti-cancer agents alone.

LDLs, oxyLDLs and apolipoproteins are physically trapped or bind to receptors expressed in the dense network of randomly branching blood vessels and sinusoids of the tumor neovasculature and have the capacity to deposit or bind to LDL receptors on the tumor endothelium and to scavenger recepors on macrophages OxyLDL or apolipoproteins bound to tumor endotheium or macrophages they induce apoptosis or they promote inflammation by activating vascular cells and macrophages to generate cytokines, chemoattractants and tissue factor.

Superantigens in nucleic acid or polypeptide form are conjugated to the lipoproteins and amplify the inflammatory effect of the lipoproteins by inducing apoptosis of endothelial cells, upregulating endothelial cell integrins, adhesins and procoagulant activity while activating macrophages and immunocytes. Any tumor which is neovascularized is eligible for this therapy. These conjugates therefore have the advantages of localizing to disseminated and neovascularized tumor, inducing apoptosis and initiating a powerful anti-tumor response.

Lipoproteins

Lipoproteins are globular particles of high molecular weight that transport nonpolar lipids (primarily triglycerides and cholesterol esters) through the plasma. Lipoproteins have been classified on the basis of their densities into five major classes: chylomicrons, very low density lipoproteins (VLDL), intermediate-density lipoproteins (IDL), low-density lipoproteins (LDL), and high-density lipoproteins (HDL). The physical-chemical characteristics of the major lipoprotein classes are presented in Table The core of the spherical lipoprotein particle is composed of two nonpolar lipids hydrophobic lipids, triglyceride and cholesteryl ester, which are present in different lipoproteins in varying amounts. This hydrophobic core accounts for most of the mass of the particle, and consists of triglycerides and cholesterol esters in varying proportions. Surrounding the core is a polar surface coat of phospholipids that stabilize the lipoprotein particle so that it can remain in solution in the plasma. Variable amounts of unesterified cholesterol are interdigitated with the phospholipids of the surface coat. In addition to phospholipid, the polar coat contains small amounts of unesterified cholesterol. Each lipoprotein particle also contains specific proteins (termed apoproteins) that are exposed at the surface and extend into the core. The apoproteins bind specific enzymes or receptors on tumor microvascular cells.

Chylomicrons

Chylomicrons are large lipoprotein particles formed within intestinal epithelial cells from dietary triglycerides and cholesterol which are secreted into the intestinal lymph and pass into the general circulation where they adhere to LDL receptors on the tumor microcapillaries. Chylomicron remnants are removed by both LDL receptors and LDL-receptor related protein/alpha2-macroglobulin receptor (LRP). While bound to these endothelial surfaces, the chylomicrons are exposed to the enzyme lipoprotein lipase. The chylomicrons contain an apoprotein, apoprotein CII, that activates the lipase, liberating free fatty acids and monoglycerides.

Very Low Density Lipoprotein (VLDL)

Very low density lipoprotein (VLDL)are triglyceride rich particles which are secreted from the liver into the bloodstream after conversion of carbohydrate to glycerol-esterified fatty acids to form triglycerides. VLDL particles are relatively large, carry 5 to 10 times more triglycerides than cholesteryl esters, and contain a form of apoprotein B, designated B 100, that differs from the apoprotein B48 of chylomicrons. The VLDL particles are transported to LDL receptors on tumor microcapillaries, where they interact with the same lipoprotein lipase enzyme that catabolizes chylomicrons. VLDL also binds to the VLDL receptor via apolipoprotein E and lipoprotein lipase. Both apolipoprotein E and lipoprotein lipase are constituents of chylomicron remnants which are a physiological ligand for the VLDL receptor.

Plasma Apolipoproteins

Plasma apolipoproteins have a central role in plasma lipid transport. Central to the functions of all apolipoproteins (apo) is specialized regions termed amphiphathic helices which have the ability to bind phospholipids. The amphiphatic helices in apoA-I, apoA-II and apoC-III comprise multiple repeats of 22 amino acids or 22-mer periodicity each consisting of a tandem array of two 11-mers which tend to begin or end with a proline.

The characteristic spatial arrangement of the hydrophobic and hydrophilic amino acids e within the amphipathic helices is that the hydrophobic face is intercalated between the fatty acyl chains of phospholipids and the hydrophilic face is located close to the polar head groups of phospholipids. Such an orientation allows the interaction of protein domains with lipoprotein-modifying enzymes and cellular receptors that control the catabolism of lipoproteins (Lp) and their removal from the circulation. The major apolipoproteins useful in the present superantigen-apolipoprotein conjugates are as follows:

Apolipoprotein (a)

Apolipoprotein (a) (Lp (a)) is made by hepatocytes and is secreted into plasma where it forms a covalent linkage by a single interchain disulfide bond to a unique multikringle glycoprotein, with apo B 100 of LDL to form lipoprotein(a). called aploliprotein(a). Protein apo (a) has structural similarities to plasminogen and consists of multiple bent repeats of amino acid sequences. Apolipoprotein (a) exists in polymorphs distinguished by molecular weights. The molecular basis for the size variation of apo[a] is primarily due to multiple apo[a] alleles that differ in the number of kringle type 2 (plasminogen kringle type 4) repeats. Minor variability in apo(a) size might be due to differences in glycosylation, as carbohydrates make up 25-40% of the apo (a) weight.

A close structural similarity exists between apo(a) and plasminogen a protease zymogen whose active form cleaves fibrin to dissolve blood clots, is activated by tissue and urokinase plasminogen activators via cleavage at a specific arginine residue. Indeed, in-vitro and ex-vivo studies have shown that apo(a) binds to immobilized fibrin (fibrinogen), to the plasminogen receptor on endothelial cells and competes with tissue plasminogen activator in converting plasminogen to plasmin. Lipoprotein(a) also competes with plasminogen for its high-affinity binding sites in endothelium, platelets, and macrophages. Because of structural homology with plasminogen apo(a)I competively inhibits fibrin-dependent activation of plasminogen to plasmin and plasmin-mediated activation of cytokine transforming growth factor-b. Hence, Lp(a) is capable of interfering with the fibrinolytic process by acting as a procoagulant. The colocalization of apo(a) with fibrin (fibrinogen) in the arterial wall further suggests that Lp(a) is thrombogenic.

Lp(a) is a poor ligand for the LDL receptor and is consequently taken up and degraded by unregulated mechanisms, leading to tissue accumulation. Lp(a) is targeted to uptake by macrophages, presumably through the scavenger-receptor pathway. Owing to the lower B-carotene content, Lp(a) may be more easily oxidized than LDL. Oxidized Lp(a) such as Lp(a) modified by malondialdehyde, a product generated in vivo from aggregated platelets, is avidly taken up by monocyte-macrophages. through the scavenger-receptor pathway. Lp(a) accumulates in either the arterial wall and in vein grafts, respectively suggesting that Lp(a) can also traverse the endothelium of arterial vessels and reach the intima by non-receptor-mediated mechanisms and that this transport process is influenced by the density/size of Lp(a). There, Lp(a) can form complexes with such tissue-matrix components as proteoglycans, glycosaminoglycans, and collagen as well as fibrin. The magnitude of the transfer of Lp(a) from the plasma compartment to the arterial wall is larger when plasma Lp(a) levels are elevated because of a gradient effect or because of a possible direct action of Lp(a) on arterial permeability.

Apolipoprotein B

Apolipoprotein B occurs in two forms termed apoB-100 and apoB-48. In humans apoB-48 is produced only by the intestine and apolipoprotein B-100 originates from the liver. Apolipoprotein B-100, which contains 4536 amino acid residues, is the major apolipoprotein of VLDL, IDL, Lp(a) and is the sole apolipoprotein of LDL. ApoB-48 consists of the amino-terminal half of apoB-100, contains 2152 amino acid residues and is devoid of binding domain for the LDL receptor.

Apolipoprotein E4

Apolipoprotein (apo) E is a 34-kCa protein coded for by a gene on chromosome 19 and plays a prominent role in the transport and metabolism of plasma cholesterol and triglyceride through its ablity to interact with the low density lipoprotein (LDL) receptor and the LDL receptor related protein (LRP). Apolipoprotein E (apoE) is a 34-kda protein component of lipoproteins that mediates their binding to the low density lipoprotein (LDL) receptor and to the LDL receptor-related protein (LRP). Apolipoprotein E is a major apolipoprotein in the nervous system, where it is thought to redistribute lipoprotein cholesterol among the neurons and their supporting cells and to maintain cholesterol homeostasis. Apart from this function, apoE in the peripheral nervous system functions in the redistribution of lipids during regeneration.

Oxidized LDL

LDL is also rapidly transported across an intact endothelium and becomes trapped in the three-dimensional cage work of fibers and fibrils secreted by the artery wall cells. This concentration-dependent process does not require receptor-mediated endocytosis. LDL entrapped in arteries or bound to receptors on endothelium or the tumor microcirculation undergoes diverse enzymic and chemical modifications. It can also be introduced into the cell a variety of lipophilic invaders such as lipid peroxidation products and cholesterol oxides that may irreversibly modify cellular functions. The early oxidative modification of the trapped LDL in vivo occurs before monocytes are recruited and results in the oxidization of lipids in LDL with little change in apoB.

Monocytes recruited to the lesion, are converted into macrophages and the LDL lipids are further oxidized. Once the LDL contains fatty acid lipid peroxides, there follows (especially in the presence of metal ions) a rapid propagation that amplifies dramatically the number of free radicals and leads to extensive fragmentation of the fatty acid chains with the generation of a broad spectrum of oxysterols, shorter-chain aldehydes (e.g., malondialdehyde and 4-hydroxynonenal) some of which involve the covalent binding of short-chain substituents to the amino groups of lysine residues in apoprotein B (and possibly to other portions of the apoprotein B molecule) masking lysine 6-amino groups. Acetyl LDL and scavenger receptors recognize modifications effected by chemical acetylation and highly oxidized LDL.

Incubation of LDL with endothelial cells, smooth muscle cells, and macrophages in vitro induces oxidation of polyunsaturated fatty acids. Lipid peroxides formed fragment fatty acyl chains and attach covalently to apoB or fragments thereof, thereby rendering the modified particles competent for endocytosis by the scavenger receptor. LDL particles also undergo peroxidation of polyunsaturated fatty acids which produces oxidative modification and conversion of LDL lecithin to lysolecithin.

Modification of LDL with malondialdehyde, a product of arachidonic acid metabolism or oxidation of LDL leads to foam cell formation. Unlike native LDL, oxidized LDL is mitogenic or induces apoptosis in arterial endothelial and smooth muscle cells. It also induces endothelial cells and monocytes to express high levels of tissue factor and plasminogen activator inhibitor. Levels of P-selectin are increased intracellularly and are released by oxy-LDL which can also directly stimulate PDGF production in endothelial cells. Oxidized LDL also induce the expression of endothelin, to inhibit the expression of nitric oxide synthase, and to inhibit the resulting vasodilation. Platelet accumulation and local increases in thromboxane A, serotonin, ADP, platelet activating factor, and activated thrombin, together with a local reduction in prostacyclin further contribute to a procoagulant state.

Another stable end product of cellular oxidative modification of LDL is lysophosphatidylcholine, which is generated by phospholipase A2 hydrolysis. This lipid selectively induces the expression of adhesion molecules for monocytes, vascular cell adhesion molecule-1 (VCAM-1), and ICAM-1 in cultured human arterial endothelial cells. TNF-a activation is a prerequisite for the observed lysophosphatidylcholine induction of VCAM-1. Lysophosphatidylcholine also induces monocyte chemotaxis, arrests macrophage migration and induces macrophage proliferation through SR-A-mediated internalization of modified lipoprotein. Finally, lysophosphatidylcholine induces gene expression for smooth muscle/fibroblast growth factors, the A and B chains of PDGF, and heparin-binding epidermal growth factor-like protein in cultured endothelial cells.

Oxy LDL Mimics

The cytotoxic effects of highly oxidized LDL are mimicked by higher concentrations of oxysteroid. particularly 7β-hydroperoxycholesterol. 7β-hydroxycholesterol, 7-ketocholesterol and 5α-6α-epoxycholesterol. These oxysterols can induce apoptosis in a variety of cells. Of these end products, 7β-hydroperoxy-choles-5-en-3B-ol has been identified as the primary cytotoxin in highly oxidized LDL. This molecule accounts for approximately 90% of the cytotoxicicy of lipids extracted from highly oxidized LDL in vitro. Fatty acid hydroperoxides and aldehydes found in oxidized LDL also alter intracellular functions. For example, 4-hydroxynonenal (4-HNE). a component of oxidized LDL, induces binding of the coagulation protein, Factor Xa to endothelial cells. In addition, oxidized LDL and mm-LDL can significantly induce the release of IL-1 from macrophages. Saponified Cu²+-oxidized LDL and mm-LDL have been shown to contain 9-HODE, 13-HODEB, and cholesterol-9-HODE, which increase IL-1 release from macrophages. 4-HNE also causes a variety of effects on monocytes, including stimulation of monocyte migration through induction of chemoattractant proteins and initiation of apoptosis

Mildly Oxidized LDL (mm-LDL)

Mildly oxidized LDL (mm-LDL) induces elevated levels of cAMP by a G protein-mediated mechanism and induces inflammatory molecules both by increasing the rates of gene transcription and by stabilizIng the mRNA for these genes. Exposure of the arterial wall to (mm-LDL) or biologically active products of lipid peroxidation results in binding to the LDL-R. mm-LDL also induces monocytes to bind to endothelial cells, and induces changes which affect monocyte binding, tethering, activation, and attachment. mm-LDL also induces an inflammatory phenotype in endothelial cells and proinflammatory cytokines accompanied by increase the levels of the transcription factor, NF kB, which has been linked to the expression of a variety of adhesion molecules. In particular, lysophosphosphatidylcholine, a product of LDL oxidation, has been shown to be a chemoattractant for monocytes and T-lymphocytes, to induce the adhesion molecules VCAM-1 and ICAM-1, and to increase levels of PDGF and heparin-binding epidermal growth factor mRNA in endothelial cells and smooth muscle cells. Increases in ICAM-1 expression lead to enhanced monocyte adhesion to the vessel wall.

Moreover, mm-LDL induces endothelial cells to produce the potent monocyte activators a monocyte chemoattractant protein 1 (MCP-1) and monocyte colony stimulating factor (M-CSF). Macrophage Class A scavenger receptors and CD36, a Class B scavenger receptor are up-regulated by M-CSF. Once bound to specific scavenger receptors, mm-LDL can initiate cell signaling events in vascular cells stimulating phosphoinositide metabolism and calcium flux as well as stimulate phospholipase E1 activity through a tyrosine kinase-dependent mechanism independent of protein kinase C. This induces the release of phosphatidic acid or arachidonic acid for eicosanoid production in the vessel a wall. A portion of this activity may be mediated by the Class A scavenger receptor ligands which stimulate macrophage urokinase expression and IL-1 production a growth factor for smooth muscle cells.

The biological properties of the lipids in mildly oxidized LDL differ from those induced by the lipids in highly oxidized LDL. For example, the expression of tissue factor by endothelial cells is induced by mildly oxidized LDL but not by highly oxidized LDL. The lipids in highly oxidized LDL are cytotoxic, whereas the lipids in mildly oxidized LDL are not. Mildly oxidized LDL induced the activation of the NFkB-like transcription factor and the increase in the appearance of specific oxidized phospholipids. With continued oxidation, highly oxidized LDL such as lysophosphatidylcholine and oxidized sterols are produced with different biological activity as given above.

The ability of mm-LDL to induce monocyte adherence to endothelial cells is mimicked by three polar bioactive lipids isolated from mm-LDL as well as oxidized 1-palmitoyl-2-arachidonyl-sn-glycerophosphocholine. The molecular structure of two bioactive lipids were identified

1-palmitoyl-2-(5-oxovaleryl)-sn-glycero-3-phosphocholine (m/t 594.3) and 1-palmitoyl-2-glutaryl-sn-glycero-3-phosphocholine (m/t 610.2). The third lipid (m/t 831) has tentatively been described as an arachidonic acid-containing phospholipid containing three or four oxygen molecules, potentially forming a conjugated triene structure characteristic of leukotrienes. The latter serves as a substrate for paraoxonase, and those with fragmentation products such as 5-oxyvalerate at the sn-2 position may represent substrates for PAF acetylhydroxylase.

Glycated LDL

Glycated LDL is recognized less well by the LDL receptor, but is taken up more rapidly by macrophages. Very prolonged exposure of LDL to high concentrations of glucose leads to glucose-mediated cross-linking and the generation of advanced glycosylation end products, which macrophages recognize in a specific saturable fashion.

Artificial Complexes of LDL

Artificial complexes of LDL formed by incubation with fibronectin, heparin, and fibrillar collagen are also candidates, and the uptake there appears to be through recognition of the fibronectin. Complexes of LDL with itself are taken up more rapidly than native LDL via the LDL receptor. After incubation with neutrophils LDL is taken up more rapidly by macrophages. This is attributable to the dimerization of LDL by the action of secreted neutrophil elastase on native LDL

Apoprotein Genes

The genes for the major apoproteins associated with the lipoproteins have been cloned. These include apolipoprotein (a) (McLean J W Nature 330: 132-137 (1987)), apolipoprotein B-100 (Chen S H J. Biol Chem. 261: 12918-12921 (1986)), apolipoprotein E4 provided by Drs. S Lauer and J. Taylor. Lp(a) has been cloned from cDNA libraries constructed from human liver mRNA (McLean J W Nature 330: 132-137 (1987)). Complete sequence analysis of a 14 000-base-pair (bp) DNA copy of apo(a) mRNA showed many exact or nearly exact repeats of a 342-bp sequence occurred. Indeed, most of the mRNA consist of 22 tandem exact repeats and 15 modified repeats. Apolipoprotein (a) belongs to a gene family that includes genes encoding clotting factors, structural proteins, and growth factors. Domains shared by these proteins are protease-like domains, kringle units, calcium binding domains, and epidermal growth factor precursor domains.

In the present invention, superantigens are ligated to the major classes of lipoproteins in human plasma including LDL, IDL, HDL, VLDL, chylomicons and remnants containing apoproteins and mm-LDL, oxy LDL isosterols, inositols, lysophosphatidlycholine, synthetic mimics of LDL activity and oxyLDL byproducts by methods given in Example 47 Because of their unique capacity to adhere to tumor microvasculature and evoke an apoptotic/inflammatory/prothrombotic response, the lipoprotein structures preferred for ligation to SAg include but are not limited to Lp(a), LpB-1000 or B-47, oxyLDL, oxyLDL byproducts, oxyLDL mimics and IDL.

The lipoproteins used for conjugation are prepared as in Examples 48-49. The superantigens used for conjugation are preferentially in nucleic acid or phage form but may also be in peptide, polypeptide nucleic acid or phage display form. They are coupled to the various LDL, oxyLDL or apolipoproteins via methods given in Examples 3, 5, 47. Alternatively, SAg are incorporated or bound or conjugated to a vesicular, exosomal structures shed from normal, tumor or sickled cells expressing LDL, oxy LDL, oxyLDL mimics or apolioproteins. Superantigens are also integrated into liposomal structures prepared to express natural or synthetic LDL, oxyLDL, apolipoprotens or oxyLDL mimics as described in Section 45 and Examples 3, 5, 6, 36, 42. Optionally, integrin ligand sequences such as RGD are added to facilitate the localization of the conjugates to the tumor microvasculature binding to the aᵥb₃ integrin and aᵥb₅ integrin which are expressed therein (see Example 6). These superantigen-lipoprotein conjugates are physically trapped in the dense network of randomly branching blood vessels of the tumor microcirculation and also bind to LDL or scavenger receptors expressed in the tumor neovasculature.

Constructs consisting of naked Sag nucleic acids containing CpG backbone fused to a apoprotein nucleic acids alone or incorporated into liposomes are prepared as in Example 3, 6, 14, 30-31 and delivered to the tumor sites in vivo as in Examples 14, 30-31.

These constructs are useful in vivo as a therapeutic antitumor vaccines according to Examples 14, 15, 16, 18-23. They are also useful ex vivo for producing a population of tumor specific effector T or NKT cells for adoptive immunotherapy of cancer (Examples 2-5, 7, 15, 16, 18-23).

Tumor Cells or Sickled Erythrocytes and Vesicles Expressing SAg and Apolipoproteins

Superantigen nucleic acids are fused in frame to nucleic acids encoding apoproteins including but not limited to apoproteins Lp(a), B-48 and 100 and E3 and transfected into tumor cells in vivo to produce tumor cells expressing superantigens and apoproteins. These tumor cells are recognized by apoprotein receptors in tumor microvasculature. Tumor cells are also transfected ex vivo with the identical nucleic acid constructs. A RGD a sequence is added to promote deposition in the tumor microvasculature which are useful. These tumor cell transfectants expressing Sag, apoprotein and RGD bind to apoprotein receptors and integrins respectively expressed in tumor microvasculature wherein they initiate a potent and localized anti-tumor response.

Superantigen nucleic acids together with nucleic acids encoding either apo(a), apoB and apoE4 are also transfected into nucleated sickled erythrocytes (e.g., proerythroblast or a normoblast phase) by methods given in Examples 1 and 6. The integrin ligand RGD nucleic acids are transfected into tumor cells or sickled cells to facilitate the localization of the transfected tumor cells and sickled cells to integrins expressed in the tumor neovasculature in vivo (see Example 6). Alternatively, the sickled erythrocytes or tumor cells acquire the apolipoprotein or oxyLDL by coculture with liposomes which express the apolipoprotein or oxyLDL (see Section 7 & Example 5).

These tumor cells or sickle cell transfectants are adminstered parenterally and are capable of trafficking to tumor microvascuature wherein they bind to apolipoprotein and scavenger receptors on endothelial cells and macrophages. The transfectants are phagocytosed by macrophages cells and induce endothelial cell apoptosis. SAgs expressed on the tumor cells and sickle cells also induce a local T cell inflammatory anti-tumor response which envelops the neighboring tumor cells.

These tumor cell and sickle cell constructs are prepared by methods given in Examples 1 & 6 and are useful in vivo against primary and/or metastatic tumors according to Examples 14, 15, 16, 18-23.

Tumor Cells & Endothelial Transfected in Vivo with SAg and Lipoprotein Receptors or Oxidized Lipoprotein Receptors

The genes encoding the LDL oxyLDL, VLDL, LRP, CD36, SREC and LOX-1receptors as well as macrophage scavenger receptors, expressed on endothelial cells and macrophages and have been cloned. Nucleic acids encoding receptors for various apolipoproteins including but not limited to the LDL or apo a, apoB or apo E receptor, CD36 receptor, LRP receptor, macrophage scavenger receptor, endothelial cell oxyLDL receptor (LOX-1) and endothelial cell scavenger cell receptor (SREC) alone or together with nucleic acids encoding superantigens are injected directly into tumor sites. The same nucleic acids are transfected into tumor cells in vivo. Transfection of these receptors into tumor cells and tumor microvascular endothelial cells results in the expression of the LDL receptor protein with high affinity binding specificity for LDL oxyLDL and Lp(a). Exposure of the transfected tumor cells or endothelial cells to exogenously introduced oxidized LDL (especially sterol and lysocholinephosphatidic acid) induces tumor endothelial cell apoptosis analogous to that seen in endothelial cell after exposure to oxyLDL. The transfected tumor cells internalize and degrade the oxyLDL and because they, like macrophages, have no means of down regulating the scavenger receptor are transformed to “foam cells” and undergo apoptosis.

LDL Receptor (LDL-R)

The high affnity receptor for LDL known as the apoB receptor or the LDL receptor (LDL-R) found on tumor microvascular cells as well as hepatic cells and macrophages binds LDL, VLDL and chylomicron remnants via their associated apoproteins. Apolipoprotein B-100 gene has been cloned (Chen S H J. Biol Chem. 261: 12918-12921 (1986)). The LDL gene is more than 45 kilobases in length and contains 18 exons. Thirteen of the 18 exons encode protein sequences that are homologous to sequences in other proteins: five of these exons encode a sequence similar to one in the C9 component of complement; three exons encode a sequence similar to a repeat sequence in the precursor for epidermal growth factor (EGF) and in three proteins of the blood clotting system (factor IX, factor X, and protein C); and five other exons encode nonrepeated sequences that are shared only with the EGF precursor. Tβ LDL receptor appears to be a mosaic protein built up of exons shared with different proteins, and it therefore belongs to several supergene families (Sudhof T C et al., Science 228: 815-22 (1985)).

Regulation of LDL-R expression occurs primarily at the transcriptional level and is controlled by levels of free cholesterol in the cell. Inflammatory mediators such as growth factors and cytokines can promote the binding and uptake of LDL. These mediators include PDGF, TGF-β basic fibroblast growth factor, TNFα, and IL-1. Some of these mediators, such as TNF-α and IL-1, affect transcriptional regulation of the LDL-R gene at the level of the promoter.

VLDL Receptor

The VLDL receptor has been described as a new member of the LDL receptor supergene family that specifically binds VLDL and chylomicron remnants via apolipoprotein E and lipoprotein lipase. Both apolipoprotein E and lipoprotein lipase are constituents of chylomicron remnants, and a physiological ligand for the VLDL receptor (Niemeier A et al., J. Lipid Res. 37: 1733-42 (1996)).

LRP Receptor

The alpha 2-macroglobulin receptor or lipoprotein receptor-related protein (LRP) (LRP) is a cell-surface glycoprotein of 4525 amino acids that functions as a multifinctional receptor which binds and rapidly internalizes several plasma proteins. These include alpha 2-macroglobulin-protease complexes, free plasminogen activators as well as plasminogen activators complexed with their inhibitors, and beta-migrating very low density lipoproteins complexed with either apolipoprotein E or lipoprotein lipase tissue and urokinase-type plasminogen activators, plasminogen activator inhibitor-1, lipoprotein lipase, and lactoferrin. The active receptor protein is derived from a 600-kDa precursor, encoded by a 15-kb mRNA, cloned and sequenced in human, mouse, and chicken. The entire human gene (LRP1) coding for A2MR/LRP has been cloned. The gene covers about 92 kb and a total of 89 exons, varying in size from 65 bases (exon 86) to 925 bases (exon 89) have been identified. The introns vary from 82 bases (intron 53) to about 8 kb (intron 6). In the introns, 3 complete and 4 partial Alu sequences have been identified. Interexon PCR from exon 43 to 45 yielded a fragment of 2.5 kb. Attempts to subclone this fragment yielded inserts ranging between 0.8 and 1.6 kb. Sequencing of 3 subclones with different-size inserts revealed a complex repetitive element with a different size in each subclone. In the mouse LRP gene this intron is much smaller, and no repetitive sequence was observed. In 18 unrelated individuals no difference in size was observed when analyzed by interexon PCR (Van leuven, F et al., Genomics 24: 78-89 (1994))

The LRP receptor is mainly responsible for the binding and internalization of chylomicron remnants as well as apoE-containing HDL. ApoE-containing lipoproteins are taken up and degraded by receptor-mediated endocytosis. Apolipoprotein E3- and apoE4-containing lipoproteins have a similar binding affinity and cause a similar degree of lipoprotein internalization via the LDL-R and the LRP. LRP can mediate the degradation of tissue factor pathway inhibitor (TFPI), a Kunitz-type plasma serine protease inhibitor that regulates tissue factor-induced blood coagulation

The 3 9-kDa receptor-associated protein (RAP) associates with the multifinctional low density lipoprotein (LDL) receptor-related protein (LRP) and thereby prevents the binding of all known ligands, including alpha 2-macroglobulin and chylomicron remnants. RAP is predominantly localized in the endoplasmic reticulum and functions as a chaperone or escort protein in the biosynthesis or intracellular transport of LRP. RAP promotes the expression of functional LRP in vivo and stabilizes LRP within the secretory pathway.

Macrophage Scavenger Receptors

Scavenger receptors mediate the endocytosis of chemically modified lipoproteins, such as acetylated low density lipoprotein (Ac-LDL) and oxyLDL. Functional MSR are trimers of two C-terminally different subunits that contain six functional domains. The MSR gene has been cloned in an 80-kilobase human and localized to band p22 on chromosome 8 by fluorescent in situ hybridization and by genetic linkage using three common restriction fragment length polymorphisms. The human MSR gene consists of 11 exons, and two types of mRNAs are generated by alternative splicing from exon 8 to either exon 9 (type II) or to exons 10 and 11 (type I). The promoter has a 23-base pair inverted repeat with homology to the T cell element. Exon 1 encodes the S-untranslated region followed by a 1 2-kilobase intron which separates the transcription initiation and the translation initiation sites. Exon 2 encodes a cytoplasmic domain, exon 3, a transmembrane domain, exons 4 and 5, an alpha-helical coiled-coil, and exons 6-8, a collagen-like domain. The position of the gap in the coiled coil structure corresponds to the junction of exons 4 and 5 The human MSR gene consists of a Macrophage scavenger receptors (MSR) mediate the binding, internalization, and processing of a wide range of negatively charged macromolecules. Functional MSR are trimers of two C-terminally different subunits that contain six functional domains. The MSR gene has been cloned in an 80-kilobase human and localized to band p22 on chromosome 8 by fluorescent in situ hybridization and by genetic linkage using three common restriction fragment length polymorphisms. The human MSR gene consists of 11 exons, and two types of mRNAs are generated by alternative splicing from exon 8 to either exon 9 (type II) or to exons 10 and 11 (type I). The promoter has a 23-base pair inverted repeat with homology to the T cell element. Exon 1 encodes the S-untranslated region followed by a 1 2-kilobase intron which separates the transcription initiation and the translation initiation sites. Exon 2 encodes a cytoplasmic domain, exon 3, a transmembrane domain, exons 4 and 5, an alpha-helical coiled-coil, and exons 6-8, a collagen-like domain. The position of the gap in the coiled coil structure corresponds to the junction of exons 4 and 5. The human MSR gene consists of a mosaic of exons that encodes the functional domains. Furthermore, the specific arrangement of exons played a role in determining the structural characteristics of functional domains (Emi M et al., J. Biol. Chem. 268: 2120-5 (1993)).

Scavenger receptors on tumor endothelium and stroma bind oxidized LDL, apoptotic cells, and anionic phospholipids. Class A receptors, includes the type I and II macrophage scavenger receptors (SR-M and SR-MI). They are found predominantly on macrophages and activated smooth muscle cells. SR-M and SR-MI are homotrimeric membrane proteins, which are derived from alternatively spliced mRNA products of a single gene. Ligands for class A receptors include acetylated LDL, oxidized LDL, fucoidan, and carrageenan. The second class, Class B scavenger receptors, includes CD36 and SR-E1, which are found in adipose tissue, lung, liver, and macrophages.

Acetyl LDL Receptor

Acetyl LDL receptor or the scavenger receptor, is distinct from the LDL receptor and does not recognize native LDL. It has been found on tumor microvascular cells as well as monocyte/macrophages, Kupfer's cells, and endothelial cells, particularly the sinusoidal endothelial cells in the liver. The same receptor also recognizes other chemically modified forms of LDL, including acetoacetyl LDL and malondialdehyde-conjugated LDL. The acetyl LDL receptor binds OXLDL LDL modified by incubation with cultured endothelial cells. LDL incubated with cultured endothelial cells for 12 to 18 hours, undergoes a physical and chemical changes and the resulting endothelial cell-modified form of LDL is taken up by cultured macrophages 10 times more rapidly than native LDL. Thus, all three the major cell types in the artery wall can convert LDL to a form recognized by the acetyl LDL receptor.

CD36 Receptor

CD36, a multigland glycoprotein structurally related to SR-BI and CLA-1 found on monocytes, endothelial cells is a high affinity receptor for the native lipoproteins HDL, LDL, VLDL and for OXLDL and AcLDL. The CD36 gene has been cloned (Endemann G et al., J. Biol. Chem. 268:11811-6 (1993)).

Endothelial Receptors for OxyLDL: The LOX-1 Receptor (C-Type Lectin Receptor)& Scavenger Receptor Expressed by Endothelial Cells (SREC)

Endothelial dysfunction or activation elicited by oxidatively modified low-density lipoprotein (Ox-LDL) is characterized by intimal thickening and lipid deposition in the arteries. Ox-LDL and its lipid constituents impair endothelial production of nitric oxide, and induce the endothelial expression of leukocyte adhesion molecules and smooth-muscle growth factors. Vascular endothelial cells in culture and in vivo internalize and a degrade Ox-LDL through a putative receptor-mediated pathway that does not involve macrophage scavenger receptors.

LOX-1 Receptor

LOX- 1, a novel receptor for oxy-LDL, is a membrane protein that belongs structurally to the C-type lectin family, and is expressed in vivo in vascular endothelium and vascular-rich organs. The LOX-1 receptor from vascular endothelial cells has been cloned a (Hoshikawa H et al., Biochem. Biophys. Res. Commun. 245: 841-6 (1998)). Mouse LOX-1 is composed of 363 amino acids with a C-type lectin domain type II membrane protein structure and triple repeats of the sequence in the extracellular “Neck domain,” which is unlike human and bovine LOX-1. LOX-1 binds oxidized LDL with two classes of binding affinity in the presence of serum. The binding component with the higher affinity showed the lowest value of Kd among the known receptors for oxidized LDL. With respect to ligand specificity, LOX-1 is a receptor for oxy-LDL but not for Ac-LDL and recognizes a protein moiety of oxy-LDL with a ligand specificity that is distinct from other receptors for oxy-LDL, including class A and B scavenger receptors.

Scavenger Receptor Expressed by Endothelial Cells (SREC),

The primary structure of the SREC molecule has no significant homology to other types of scavenger receptors, including the LOX-1 receptor. The cDNA encodes a protein of 830 amino acids with a calculated molecular mass of 85, 735 Da (mature peptide). The cloned has an N-terminal extracellular domain with five epidermal growth factor-like cysteine pattern signatures and long C-terminal cytoplasmic domain (391 amino acids) composed of a Ser/Pro-rich region followed by a Gly-rich region (Adachi H et al., J. Biol. Chem. 272:31217-20 (1997)

The SREC mediates the binding and degradation of acetoacetylated (AcAc) and acetylated (Ac) low density lipoproteins (LDL). Isolated sinusoidal endothelial cells from the rat liver show saturable, high affinity binding of AcAc LDL and degrade AcAc LDL 10 times more effectively than aortic endothelial cells. Specific sinusoidal endothelial cells bearing the SREC not the macrophages of the reticuloendothelial system, are primarily responsible for the removal of these modified lipoproteins from the circulation in vivo. For this reason, the SREC receptor and the LOX-1 receptors are preferred for use in transfecting tumor cells tumor endothelium in vivo.

Polypeptide or naked DNA encoding receptors for LDL oxyLDL, VLDL, LRP, CD36, SREC, LOX-1 and macrophage scavenger receptor (collectively o-LDL receptors) are used individually or together with SAg polypeptidee or naked DNA containing the CpG backbone are prepared as in Examples 1, 2, 3, 30-31. Alternatively, SAg are incorporated or bound or conjugated to vesicular or exosomal structures shed from cells expressing the LDL, oxy LDL receptors. Superantigens are also incorporated into liposomal structures which express natural or synthetic LDL, oxyLDL receptors as described in Section 45 and Examples 3, 5, 6, 36, 42. All of these constructs are administered in vivo by any route but preferably by intratumoral injection as in Examples 2, 6, 14, 30-31. Once localized, and expressing o-LDL receptor(s) in tumor sites in vivo, lipoprotein preparation(s) containing their respective ligands are administered to the host. These LDL, oxyLDL or lipoproteins are non toxic to the host generally but upon binding to a dense population of receptors in the tumor induce apoptosis of tumor cells and endothelial cells expressing the receptors and initiate a well localized anti-tumor response. The presence of the SAg at the same site amplifies the immune and inflammatory anti-tumor effect. The advantage of this system is the minimal toxicity to the host since the o-LDL receptors are of host origin and the lipid infusions consist of substances which are indigenous to the host. These constructs are useful in vivo against primary or metastatic tumors according to Examples 14, 15, 16, 18-23.

50. SAg Combined with Tumor Viruses (Nucleic Acid or Peptide Forms)

SAgs are chemically conjugated to HPV-E6 or 7 human papilloma virus tumor antigens by methods given in Examples 3. Alternatively, the naked nucleotides containing immunostimuulatory sequence of the superantigen and the HPV-E6 or E7 are prepared individually or as a fusion nucleotide or protein as in Examples 5, 30, 31. Alternatively, the the SAg-HPV fusion gene is transfected into tumor cells as given in Example 1. In this case, the virus serves as the vector for tranfecting the cells with the superantigen nucleic acids. The superantigen-HPV-E6 or E7 conjugates, fusion proteins, naked DNA fusions or tumor cells expressing the superantigens and HPV are used as preventative or therapeutic vaccines under protocols given in Examples 14, 15, 16, 18-23, 30, 31. Further, SAg and HPV-E6 or E7 transfected tumor cells are subjected to irradiation or other apoptosis inducing agents or stimuli arter which the apoptotic tumor cell transfectants are presented to dendritic cells ex vivo which ingest the apoptotic tumor cells. of In the dendritic cells, the viral antigens and superantigen undergo cross priming to the class I pathway and these dendritic cells are then harvested and administered to the tumor bearing host as given in Examples 26-28. The DNA and RNA from these SAg and HPV- E6 or E7 transfected tumor cells or dendritic cells is extracted and utilized for in vivo therapy as in Examples 30-34. While the HPV-E7 is exemplified herein, the method is applicable to other viruses which are known to be associated or etiopathogenic in the malignant state including but not limited to adenovirus, EB virus, herpesvirus, hepatitis B, cytomegalovirus and Kaposi's sarcoma herpesvirus.

51. Augmented Immune Response to Cancer and Infectious Diseases: Deletion or Inactivation of Immunocyte Inhibitory Receptors and Immunoreceptor Tyrosine Based Inhibitory Motifs (ITIMs)

Many lipid-based tumor associated antigens consist of lipids, glycolipids, gangliosides, sphingolipids and lipopeptides (collectively LBTAAs) which are weak immunogens and fail to evoke an effective tumoricidal immune response. The same may be said for lipid based antigens associated with infectious organisms. For example, tumor gangliosides shed from the cell or on tumor cell surface are actually capable of suppressing T cell function. The present invention provides inhibitory receptors and their inhibitory motifs which recognize 1) lipid-based tumor associated antigens (LBTAAs), 2) lipid-based infectious disease associated antigens (LBIDAs) and 3) superantigens or self molecules associated with superantigens (SSMAS). Deletion or inactivation of these inhibitory receptors or their inhibitory motifs by pharmacologic or genetic methods results in an enhanced cellular response to tumors or infectious disease assocociated organisms. The inhibitory receptors specific for LBTAA, LBIDA and SSMAS are abbreviated as IRTAA, IRIDA and IRSAG respectively.

In the present invention, IRTAA, IRIDA and IRSAG on T cells, NK cells and NKT cells, which inhibit responses to lipid antigens presented in the context of their natural antigen presenting receptor e.g., CD1 or a suitable surrogate are deactivated or eliminated prior to exposure to specific LBTAA, LBIDA and SSMAS. The inhibitory receptors recognize and respond to the same lipid-based TAAs and self (e.g., CD1or MHC) as the activation receptors or they respond with an inhibitory signal to the activation receptor only after the activation receptor has been engaged. The immunocyte populations bearing such inhibitory receptors include but are not limited to T cells, NK cells or NKT cells. After exposure to LBTAA, the immunocyte population devoid of an IRTAA response becomes tumoricidally activated due to the unopposed stimulation or the activation receptors. This is of particular advantage in the case of weakly immunogenic LBTAAs which bind to inhibitory receptors on immunocytes. Likewise, after exposure to LBIDAs the immunocyte population devoid of IRIDAs becomes cytolytic for the infectious organism. Similarly, if a LBTAA or LBIDA is associated, genetically fused or conjugated to a SSMAS, after exposure to SSMAS and LBTAA or LBIDA the immunocyte population devoid of the IRSAG (plus IRTAA or IRIDA) becomes hyperresponsive to the LBTAA and LBIDA.

Deactivation or deletion of inhibitory receptors in T cells is carried out both ex vivo and in vivo prior to exposure to the LBTAAs by methods given in Examples 51, 52. Such measures include but are not limited to the use of specific antibody or antibody fragments directed to the inhibitory receptor(s), gene knockout by homologous recombination or exposure to an anti-sense nucleotide. Deletion of these inhibitory receptors leads to a significant enhancement of immunocyte activation in response to lipid-based TAAs and superantigens. In particular, these immunocytes with deleted Inhibitory receptors are rendered capable of responding to subdominant and dominant lipid-based TAAs on tumor cells, differentiating into Cytotoxic lymphocytes e.g., CTLs and secreting tumoricidal cytokines.

The IRTAAs and IRIDAs are found in T cells, NK cells and NKT cells fall into two structural types (1) Type I integral membrane proteins belonging to the IgG superfamily (2) Type II integral membrane proteins in the C type lectin superfamily expressed as disulfide linked dimers either as homodimers or heterodimers. Once engaged, they deactivate signals originating from the TCR CD1 activation receptor.

The present invention contemplates deletion or inactivation of the IRTAA, IRIDA, IRSAG or immune receptor tyrosine based motifs (ITIMS) These receptors induce inhibitory effects because they retain ITIM in their cytoplasmic domains which is phosphorylated and recruits SHP-1 or tyrosine phosphorylase to dephosphorylate molecules in the activation cascade. If the inhibitory receptor is engaged, it has a dominant effect and blocks cell activation of cytotoxicity and cytokine secretion.

When stimulated, the IRTAA, IRIDA, IRSAG and their respective ITIMs inhibit cellular activation by receptors specific for LBTAAs. The IRTAAs and IRIDAs on a/b TCRs confer specificity for LBTAAs and LBIDAs respectively optionally in the context of CD1 isoform. Sequence analysis of a panel of CD1-restricted, lipid-specific inhibitory TCRs reveals the incorporation of template-independent N nucleotides that encode diverse sequences and frequent charged basic residues at the V(D)J junctions. The TCR CDR3 loops containing charged residues project between the CD1 a-helices, contacting the lipid antigen hydrophilic head moieties as well as adjacent CD1 residues in a manner that explains antigen specificity and CD1 restriction.

The IRTAAs respond specifically to LBTAAs which include fatty acids, ceramides, glycolipids, sphingolipids, glycosphingolipids, phtosphingolipids, gangliosides, lipopeptides. IRIDAs recognize LBIDAs derived from bacteria, mycobacteria, parasites, fungi, protozoans or plants and respond by producing an effective immunocyte response. These antigens comprise sphingolipids, glycopeptides, phytoglycolipids, mycoglycolipids, lipoarabinans, mycolic acids, Braun's lipopeptide, inositolphosphorylceramides and plant phosphatidylinositol. Sphingolipids with inositolphosphate-containing head groups showing the general structure of ceramide-P-myoinositol-X with X referring to polar substituents consisting of ceramide-p-inositol-mannose, inositol-1-P-(6)mannose(al ,2inositol-1P-(1)ceramide, (inositol-P)2-ceramide, inositol-P-inositol-P-ceramide, inositol-P-inositol-P-ceramide are also useful. These structures are useful in native form or naturally conjugated to GPI.

Augmented Immune Response to Tumors and Infectious Diseases: Dual Inactivation or Deletion of IRTAAs or IRIDAs and SSMASs and Their ITIMs

The present invention contemplates the dual deletion or inactivation in the same immunocyte of the IRSAGs or their ITIMs and IRTAAs or IRIDAs and their respective ITIMs. Deletion or inactivation of these receptors in an immunocyte population results in augmented immunocyte responses to tumors or infectious agents after exposure to LBTAAs or LBIDAs together with SSMAS. The LBTAAs or LBIDAs are also effective when conjugated or fused to SSMAS or

free superantigen. The receptors inhibitory of superantigen activation are the killer cell inhibitory receptors (KIRs) which are a family of structurally related cell surface molecules that are expressed on subsets of human NK and T cells predominantly CD28+memory cells. These IRSAGs bind to polymorphic class I HLA-B and HLA-C alleles on a superantigen presenting cell and are functional in inhibiting cytotoxicity and cytokine production by effector T cells in response to superantigen. The p58 and p70 KIR molecules appear to recognize public epitopes formed by polymorphisms at the C-terminal portion of the a-helix of HLA-B and HLA-C alleles. In mice, KIRs recognize H-2 class I molecules and inhibit NK cell mediated cytotoxicity. KIRs are present on NK cells, subsets of T lymphocytes, including both CD4+ and CD8+ T cells.

All of the KIRs with a long cytoplasmic tail possess two immune receptor tyrosine-based inhibitory motif (ITIM) sequences (YXXL) separated by 26-28 amino acids. These motifs, which are present in other inhibitory receptors have been shown to be critical for the inhibitory signals generated by MR upon binding to a class I ligand. In their cytoplasmic domains these receptors retain ITIMs which generate a negative signal by recruiting and activating Src homology region 2 domain protein tyrosine phosphatases,

SHP-1 and SHP-2;which dephosphorylates the molecules in the activation cascade and therefore

counters the stimulatory effects of protein tyrosine kinases associated with activation pathways. The balance of the activation and inhibitory receptors for IgG and the balance between these two responses determines the cell's activities. However, engagement of the inhibitory receptor has a dominant effect and blocks the cell activation KIRs inhibit the lytic function of CTLs activated by bacterial superantigens and regulate other T cell responses since disruption of KIR recognition of self-class I molecules produces significant increases in T cell cytokine production in response to superantigen stimulation.

Deletion or Inactivation of ITIMs or Their ITAMs

Inhibitory receptors, contain immunoreceptor tyrosine based inhibitory motifs (ITIMs) in their cytoplasmic tails. In the present invention, deletion or inactivation of the ITIMs of IRTAAs, IRIDAs or IRSAGs results in augmented activation of immunocytes in response to their respective LBTAAs, LBIDAs and superantigens.

Several inhibitory receptors reside in families that have similar characteristics. These receptors are inert when self-aggregated but are able to abolish cellular signals when coligated to stimulatory receptors. Their cytoplasmic domains contain one or more ITIMs, defined by the six-amino acid sequence (SEQ ID NO: 46) (ILV)xYxx(LV). ITIM sequences are phosphorylated on receptor coligation to create a binding site for Src-homology 2 (SH2) domain-containing cytoplasmic factors that can transmit the inhibitory signal intracellularly. When phosphorylated these receptors recruit SHP-1 or tyrosine phosphorylase which dephosphorylates molecules in the activation cascade. Several families of inhibitory receptors with specificity for MHC class I molecules have been identified, all of which contain ITIM sequences in their cytoplasmic tails family.

The inhibitory receptors contain an activating receptor counterpart with a highly homologous extracellular domain and a short cytoplasmic portion that lacks signaling capacity. The transmembrane domains of these activation receptors are characterized by the presence of a charged amino acid, a hallmark of receptors that associate with accessory subunits containing the immune receptor activation motif (ITAM). ITIM-bearing receptors commonly inhibit activating signals triggered by receptors that contain the ITAM in their cytoplasmic tails, such as the BCRs, TCRs, and FeRs. Cell activation mediated by ITAM-containing receptors involves the phosphorylation and activation of e several tyrosine kinases and subsequent activation of phospholipase Cg and phosphatidylinositol 3-kinase (PLCg and P13-K), together leading to the production of phosphoinositol messengers and a sustained increase in cytoplasmic Ca²+. If the inhibitory receptor is engaged it has a dominant effect and blocks the cell activation of cytotoxicity and cytokine production. The present invention contemplates the blockade or deletion of the ITAM portion of the activation receptor as a means of blocking the inhibitory signal thus rendering the immunocyte hyperresponsive to LBTAA, LBIDA or SSMAS.

Inhibitory receptors use two different pathways to terminate cell activation depending on the type of molecule that is recruited to the phosphorylated ITIM sequences: 1) protein dephosphorylation mediated by tyrosine phosphatases SHIP and/or SH-2 which abrogate the most proximal events in the activation cascade resulting in the abolition of Ca²⁺ mobilization and 2) an inhibitory signal is generated via phosphoinositol phosphatase SHIP which hydrolyzes phosphoinositol messengers and does not affect proximal events triggered by the activating receptor, such as the activation of kinases, receptor phosphorylation, or Ca²+ release from intracellular stores. Rather, it specifically impedes extracellular Ca²+ influx and therefore blocks a sustained increase in cytoplasmic Ca²+. The KIR signal is dependent on SHP-I and not SHIP. while the FcgRII signal is dependent on the phosphoinositol phosphatase SHIP and not the tyrosine phosphatase SHP-1. Three molecules recruited by inhibitory receptors, SHP-1, SHP-2 and SHIP, are associated with antigen, Fc, growth factor, and cytokine receptors and their absence results in augmentation of cell activation and proliferation. They do not function through recruitment to inhibitory receptors, but rather via association with stimulatory receptors to set thresholds or to prevent unsolicited cell activation. The dissociation of antigen from its binding to molecules recruited by SHP-1, SHP-2 and SHIP would lead to an abortive negative signal and augmented immunocyte reactivity to TAA and superantigens.

Human KIRs contain two ITIM sequences in their cytoplasmic domain. Each of these ITIMs. in its phosphorylated form, can bind in vitro to SHP-1 and SH2 but not to SHIP. SHP-1 prefers the sequence VxYxxL. The N-terminal ITIM (SEQ ID NO: 165) (VTYAQL) binds to both of the SHP-1 SH2domains, and it does it more efficiently than the C-terminal ITIM (SEQ ID NO: 166) (IVYELL) . The N-terminal ITIM has heen found to be sufficient for the inhibitory signal in deletion studies.

Functional inactivation of ITIMs (or their SH-2, or SHIP binding sites) of IRTAA. IRIDA and IRSAG using pharmacologic agents, specific (intracellular) antibodies and gene knockouts in immunocytes are envisioned as a means of providing a population of immunocytes hyperresponsive to TAA and superantigens.

The inhibitory receptors on immunocytes are deleted or inactivated in vivo or ex vivo before exposure to lipid-based TAAs. or superantigens. In vivo inactivation of IRTAAs, IRIDAs, IRSAGs is accomplished via administration of an anitsense molecule which inactivates the relevant inhibitory ITIM or related signaling sequences. These immunocytes are activated in vivo by exposure to 1) LBTAA expressed on endogenous tumor or by exogenous purified LBTAAs 2) LBIDAs derived from infectious disease associated organisms expressed on endogenous organisms or by exogenous purified LBIDAs 3) exogenous superantigen administration. LBTAAs, LBIDAs or SSMAS are useful in vivo as vaccines or against established cancer or infectious disease as given in Example 54. Alternatively, immunocyte IRTAAs, IRIDAs, IRSAGs or their respective ITIMs are deleted or inactivated ex vivo using methods given in Examples 51, 52 after which these cells are exposed to LBTAAs, LBIDAs or SSMAS as given in Examples 53, 54. When it is desirable to augment the immunocyte response to LBTAA or LBIDAs and SSMAS. these cells are exposed to LBTAA or LBIDAs and SSMAS simultaneously or sequentially as described in Example 54. In order to prevent uncontrolled T cell activation and autoimmune responses by the immunocytes depleted of their inhibitory receptors in vivo, immunocytes with deactivated or deleted inhibitory receptors are transduced ex vivo with the HSV thymidine kinase gene rendering them susceptible to killing by gancyclivir in vivo as described in Example 56.

The present invention encompasses IRTAA, IRIDA, IRSAG represented in any receptor families as long as they retain their functional properties which when deleted or inactivated by methods given in Examples 51, 52 produce similar augmented responses to LBTAAs, LBIDAs and SSMAS as in Examples 53, 54.

52. Deletion of LBTAA, LBIDA & SSMAS Motifs which Selectively Bind and Activate IRLBTs, IRLAs and IRSAs

The structural motifs in the LBTAA, NTLB molecules and superantigens which selectively bind to IRLBTs, IRLAs or IRSAs and generate an inhibitory signal are identified. These molecules termed antagonist motifs, are deleted from the LBTAA, LBIDA and superantigens molecules so that remaining agonist motifs selectively bind and stimulate the immunocyte activating receptors (e.g. via their their receptors or ITAMs) without activating the dominant inhibitory receptor or their ITIMs. This results in enhanced signaling and activation by the immunocyte population in response to weakly immunogenic LBTAAs, LBIDAs and SSMAS or free superantigens. Alternatively, the molecules in the enzymatic chain which produce activation of the inhibitory receptor following exposure to LBTAAs, LBIDAs or superantigen e.g. SHP-1, SHIP are functionally and reversibly deactivated (e.g.,intracellular antibodies ) or deleted (e.g., gene knockout) leading to unopposed signaling by the activation receptor and hyperresponsiveness to LBTAAss, LBIDAs and SSMAS or superantigens. Alternatively, the functional sites on the ITAM sequence of the activating receptor (which are activated by ITIMs) are blocked or inactivated pharmacologically (e.g. intracellular anitbodies or anti-sense) or genetically deleted (ITAM sequence knockout) as given in Examples 51-52.

53. Immunocytes Deleted of Nucleic Acids Encoding IRLBT or IRSAG or Their ITIMs and Fas Genes

The present invention envisions an immunocyte not only depleted of IRLBTs and IRSAGs but also devoid of Fas ligand receptors. Such an immunocyte is not only hyperresponsive to LBTAAs and SSMAS or free superantigens but also unable to undergo apoptosis in response to tumor cells which secrete Fas ligand. Therefore, when used in adoptive transfer, these immunocytes continue to display their tumoricidal properties while also penetrating tumor tissue without undergoing apoptosis by Fas ligand secreting tumor cells. The deletion or functional inactivation of the Fas gene in immunocytes and T cells, NK cells and NKT cells in particular is accomplished ex vivo by homologous recombination or anti-sense as given in Examples 51 and 52. The immunocytes are useful for adoptive immunotherapy of cancer (Examples 2-5, 7, 15, 16 18-23, 54).

54. Genes Encoding IRTAA, IRIDA IRSAG, ITIMS & ITAMs

Genes encoding the inhibitory receptors and their ITIMS are cloned using techniques well defined in the art. Their size and chromosomal location are determined using well developed techniques in the field. It is predicted that they represent a 40 base pair segment. In the present invention, genes encoding IRTAAs, IRIDAs, IRSAGs and their ITIMs, or signaling sequences in immunocytes are deleted or inactivated in vivo or ex vivo by methods given in Examples 51 and 52. These cells are then exposed to IRTAA, IRIDA, IRSAG in vivo or ex vivo Example 53, 54. The ex vivo treated immunocytes are useful for adoptive immunotherapy of cancer and infectious disease (Examples 2-5, 7, 15, 16 18-23, 53, 54).

55. Therapeutic Composition Comprising Superantigen or SSMAS Conjugated to LBTAAs and LBIDAs

In the present invention, superantigens are conjugated to LBTAA which include fatty acids, ceramides, glycolipids, sphingolipids, glycosphingolipids, gangliosides, lipopeptides. Superantigens are also conjugated to LBIDAs, glycan and peptidoglycan antigens derived from bacteria, mycobacteria, parasite, fungi or plants comprising sphingolipids, glycopeptides, peptidoglycans and teichoic acids, phytoglycolipids, mycoglycolipids, lipoarabinan, mycolic acids, Braun's lipopeptide, inositolphosphorylceramides and plant phosphatidylinositol. Sphingolipids with inositolphosphate-containing head groups showing the general structure of ceramide-P-myoinositol-X with X referring to polar substituents consisting of ceramide-p-inositol-mannose, inositol-1-P-(6)mannose(a1,2inositol-1P-(1)ceramide, (inositol-P)2-ceramide, inositol-P-inositol-P-ceramide, inositol-P-inositol-P-ceramide are also useful. These constructs are used in native form or they are further conjugated to GPI structures. They are also isolated from shed membranes, exosomes, or vesicles of the native organism. These lipids are extracted and purified as given in Example 55. SAg-LBTAA or SAg-LBIDA conjugates are prepared by methods given in Examples 4-5. For immunization, these constructs are used alone or they are loaded onto CD1 receptors in soluble form or on the surface of APCs as given in Example 5. These molecules especially with attached GPI are fused to cellular membranes such as tumor cells or erythrocytes by methods given in Example 5. In this form, they are used activate T cells, NKT cells or NK cells. These constructs are useful in vivo as a therapeutic antitumor vaccines according to Examples 14, 15, 16, 18-23. They are also useful ex vivo for producing a population tumoricidal T cells, NK cells or NKT cells for adoptive immunotherapy of cancer (Examples 2-5, 7, 15, 16, 18-23). They are used ex vivo to activate immunocytes in which IRTAA, IRIDA or IRSAG are deleted (knockout) or functionally deactivated (anti-sense-treated)as given in Example 53, 54. These hyperresponsive immunocytes are then infused into the host under protocols given in Examples 15, 16, 21, 23, 53, 54. Conjugates consisting of SAg and LBIDAs derved from fungal, parasitic or mycobacterial sources are also useful for the treatment of infectious diseases such as tuberculosis, leishmaniasis, trypanosomiasis as given in Example 53. They are also useful ex vivo for activating a population of immunocytes with deleted (via gene knockout) or functionally inactivated (antisense) IRIDAs specific for bacterial, fungal, parasitic or mycobacterial antigens for use in adoptive immunotherapy of infectious disease (Examples 51, 52, 53).

56. Superantigen Conjugated to Thrombospondin I and Type 1 Repeat Peptides of Thrombospondin and Other Molecules Inducing Apoptosis of Endothelial Cells.

In the present invention, superantigens are conjugated to molecules which induce apoptosis of endothelial cells. Superantigens are known to induce tissue inflammation and in the absence of effector cells or their mediators are capable of inducing endothelial cell injury. They are conjugated to thrombospondin 1 and type 1 repeat peptides of thrombospondin which also induce apoptosis of tumor neovascular endothelial cells. The native sequence KRFKQDGGWSHWSPWSSC (SEQ ID NO: 65)or the modified sequence which lacks the TGF-B activating sequence KRAKAAGGWSHWSPWSSC (SEQ ID NO: 66)equally stimulated DNA fragmentation. The basic residues and the WSXW motif are both required for optimal activity. The thrombospondin and/or the superantigen in the conjugate may be used in nucleic acid form. The superantigen polypeptide or nucleic acid in the conjugate is capable of evoking an inflammatory response in the tumor microvasculature while the thrombospondin induces apoptosis of tumor endothelial cells. Conjugations of polypeptides and/or nucleic acids are carried out by methods given in Example 4 and 5. The conjugates are prepared by chemical crosslinking using homo or heterobifunctional crosslinking agents, carbodiimide, cyanoborohydride or glutaraldehyde as given in Hermanson G. Bioconjugate Technology Academic Press, New York, N.Y. (1996). They are also prepared as fusion proteins or phage displays according to protocols given in Examples 5 and 6. They are also useful in nucleic acid form as a chimeric SAg-thrombospondin nucleic acid construct since the CD36 gene has been cloned (Wyler B et al., Thormb Hermost 70: 5001505 (1993); Armsella AL et al., J. Biol. Chem. 269: 18985-18991 (1994). The conjugates are useful as a preventative or therapeutic anti-tumor vaccine according to Examples 15, 16, 18-23. They are also used ex vivo to produce tumor specific effector cells for adoptive immunotherapy of cancer (Examples 2-5, 7, 15, 16, 18-23).

Having now generally described the invention, the same will be more readily understood through reference to the following examples which are provided by way of illustration, and are not intended to be limiting of the present invention, unless specified.

57. Removal of SE-Specific Antibodies with Anti-Idiotype Antibodies

It is well established that naturally occurring antibodies specific for SEs are present in a large percentage of human patients. During clinical trials using a fusion protein of SEA conjugated to a tumor specific antibody, antibodies specific for SEA appeared in the serum. The titer of these antibodies often rose with treatment and their appearance correlated with increased toxicity. These antibodies also interfere with the ability of the SE conjugates to induce tumoricidal effects by binding to the T cell activating epitopes of SE in the conjugates precluding the conjugate from stimulating a tumoricidal T cell response. The antibody-bound SE conjugates as large immune complexes are also readily taken up by reticuloendothelial cells and therefore diverted from targeting the tumor.

To solve this problem, antibodies specific for the idiotypic region of anti-SE antibodies are prepared by methods given in Example 57. The anti-idiotype is preferably of the Ab2□ reticuloendothelial cells which is an internal image anti-idiotype and has the capacity to mimic the antigen used to generate the SE-specific antibody. The Ab2□ antibody recognizes an Id within the anitgen binding site, a characteristic similar to Ab2□ but fails to exhibit biological mimicry of the antigen. This particular subset of anti-idiotype antibodies would be the most desirable in the present case as it would not further activate the host anti-SE response, however the others could be useful as well. The anti-Ids could also be prepared in hybridoma or genetic form and used in vivo (via transfection of autologuous antibody producing cells or implantable chambers) to provide a continuous amount of anti-id for the duration of use of the SE-conjugates).

The anti-Ids could be prepared and administered as less immunogenic FAB, FAB₂′, or Fv fragments and humanized to avoid alloimmunization. In the case of SEB, the dominant B cell epitope is the C-terminal region (aa 225-234) which may be used to isolate the major SE specific antibodies. Anti-SE idiotypic antibodies are detected and characterized as given in Example 57. The amount of anti-idiotype antibody administered would be sufficient to neutralize all the circulating SE specific antibodies. Doses would range from 100 ng to 1000 mg and could be predicted based on in vitro neutralization tests determining the amount of antibody required to bind all of the antigen in a small volume of serum. When the the level of these antibodies is undetectable, the SE-tumor specific antibody conjugate is administered in doses effective to kill the tumor. This could range from 2-15 ng/kg-1000 ug/kg of SE in the conjugate. Devoid of SE specific antibodies to neutralize T cell activating activating activity and divert the SE conjugate from the tumor site, the SE conjugate is able to target the tumor in vivo and initiate a tumoricidal response. The preferred route of injection is intravenous but other parenteral routes such as intraperitoneal, intrathecal and intratumoral may be useful as well. The preferred method of administration is via infusion although injection, sustained release formulations and microinfusion or osmotic pumps may also be useful.

58. Functional Derivatives Proteins of Peptides

All of the protein and nucleic acid compositions given herein are intended to encompass functional derivatives. Similarly, Staphylococcal enterotoxins or superantigens are intended to encompass functional derivatives of a particular superantigen or enterotoxin.

By “functional derivative” is meant a “fragment,” “variant,” “homologue,” “analogue,” or “chemical derivative”, which terms are defined below. A functional derivative retains at least a portion of the function of the native protein monomer which permits its utility in accordance with the present invention.

A “fragment” refers to any shorter peptide. A “variant” of refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from a different species, which shares a minimum amount of structure and thereby function with the reference protein. Homologues, as used herein, typically share about 50% sequence similarity at the DNA level or about 18% sequence similarity in the amnino acid sequence.

An “analogue” refers to a non-natural molecule substantially similar to either the entire molecule or a fragment thereof

A “chemical derivative” contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

The recognition that the biologically active regions of the enterotoxins, for example, are substantially structurally homologous enables predicting the sequence of synthetic peptides which exhibit similar biological effects in accordance with this invention (Johnson, L. P. et al., Mol. Gen. Genet. 203:354-356 (1886).

A common method for evaluating sequence homology, and more importantly, for identifying statistically significant similarities of the proteins, peptides and nucleic acids given herein is by Monte Carlo analysis using an algorithm written by Lipman and Pearson to obtain a Z value. According to this analysis, Z>6 indicates probable significance, and Z>10 is considered to be statistically significant (Pearson, W. R et. al., Proc. Natl Acad Sci. USA, 85:2444-2448 (1988); Lipman, D. J. et al, Science 227:1435-1441 (1985))., Synthetic peptides corresponding to the compositions and enterotoxins, are characterized in that they are substantially homologous in amino acid sequence to an enterotoxin with statistically significant (Z>6) sequence homology and similarity to include alignment of cysteine residues and similar hydropathy profiles.

1. Variants

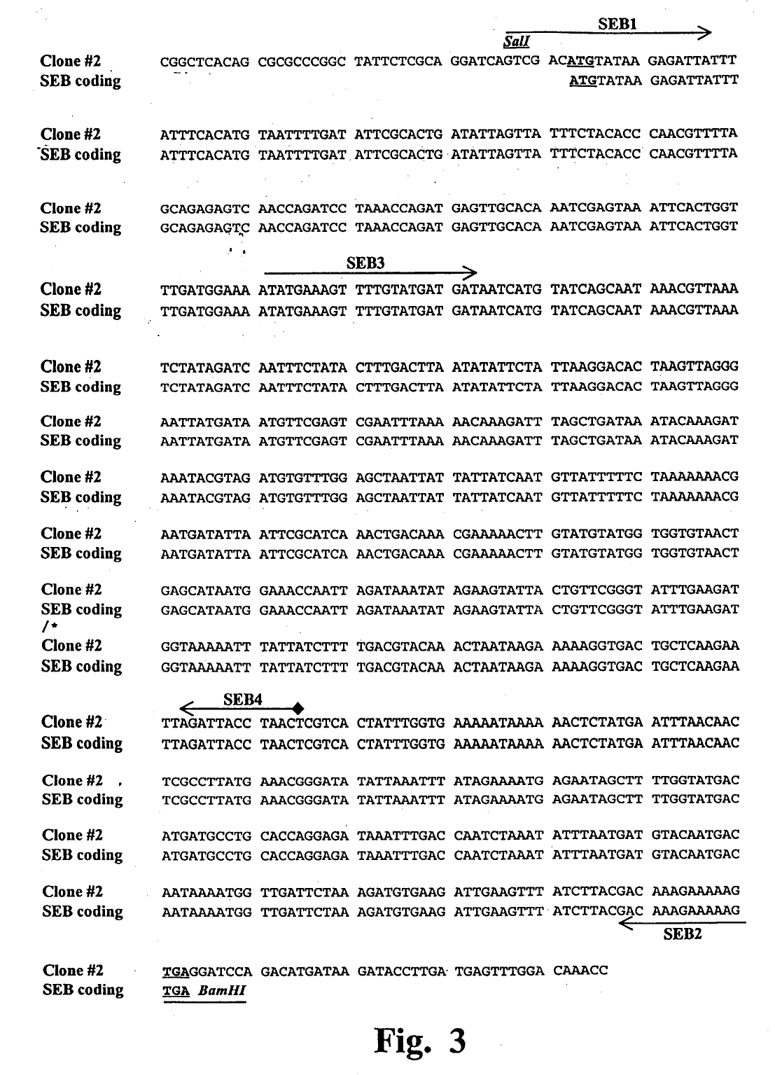

One group of variants are those in which at least one amino acid residue in the peptide molecule, and preferably, only one, has been removed and a different residue inserted in its place. For a detailed description of protein chemistry and structure, see Schulz, G. E.˜Principles of Protein Structure Springer-Verlag, New York, 1978, and Creighton, T. E., Proteins:Structure and Molecular Properties, W. H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et aL (supra) and FIGS. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);

2. Polar, negatively charged residues and their aniides: Asp, Asn, Glu, Gln;

3. Polar, positively charged residues: His, kg, Lys;

4. Large aliphatic, nonpolar residues: Met, Leu, fle, Val (Cys); and

5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

Substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, fle, Phe, Val or Ala; (c) substitution of a Cys residue for (or by) any other residue; (d) substitution of a residue having an electropositive side chain, e.g., Lys, Arg or His, for (or by) a residue having an electronegative charge, e.g., Glu or Asp; or (e) substitution of a residue having a bulky side chain, e.g., Phe, for (or by) a residue not having such a side chain, e.g., Gly.

Most deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay or biological assay as described herein. Modifications of such proteins or peptide properties as redox or thermal stability, hydrophobicity, susceptibility to proteolytic degradation or the tendency to aggregate with carriers or into multimers are assayed by methods well known to the ordinarily skilled artisan.

In the present invention, functional derivatives of proteins, peptides, enterotoxins or other related toxins and nucleic acids include synthetic polypeptides and nucleic acids characterized by substantial structural homology to enterotoxin A, enterotoxin B and Streptococcal pyrogenic exotoxins with statistically significant sequence homology and similarity (e.g, Z>6 in the Lipman and Pearson algorithm in Monte Carlo analysis (see above)).

2. Chemical Derivatives

Covalent modifications of the monomeric or polymeric forms of protein or peptide fragments thereof, of enterotoxins or peptide fragments thereof, or both are included herein. Such modifications may be introduced into the molecule by reacting targeted amino acid residues of the protein or peptide with an organic derivatizing agent that is capable of reacting with selected side chains or terminal residues. This may be accomplished before or after polymerization.

Cysteinyl residues most commonly are reacted with α-haloacetates (and corresponding amines), such as 2-chloroacetic acid or chloroacetamide, to give carboxymethyl or carboxyamidomethyl derivatives. Cysteinyl residues also are derivatized by reaction with bromotrifluoroacetone, a-bromo-(5-imidozoyl)propionic acid, chloroacetyl phosphate, Nalkylmaleimides, 3-nitro-2-pyridyl disulfide, metl)yl 2-pyridyl disulfide, p-chloromercuribenzoate, 2-chloromercuri-4- nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate; pyridoxal phosphate; pyridoxal; chloroborohydride; trinitrobenzenesulfonic acid; 0-methylisourea; 2,4 pentanedione; and transaminase-catalyzed reaction with glyoxylate.

Arginyl residues are modified by reaction with one or several conventional reagents, among them phenylglyoxal, 2,3- butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form 0-acetyl tyrosyl species and 3-nitro derivatives, respectively.

Carboxyl side groups (aspartyl or glutamyl) are selectively modified by reaction with carbodiimides as noted above. Aspartyl and glutamyl residues are converted to asparaginyl and glutaminyl residues by reaction with ammonium ions.

Glutaininyl and asparaginyl residues may be deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecule Properties, W. H. Freeman & Co., San Francisco, pp. 79-86 (1983)), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein and the like. Moieties capable of mediating such effects are disclosed, for example, in Remington's Pharmaceutical Sciences, 16th ed., Mack Publishing Co., Easton, Pa. (1980).

### EXAMPLE 1

Preparation of Plasmids for Making DNA Templates for any Gene of Interest and the Process Transfection

Mammalian oncogenes, and genes for oncogenic transcription factors, angiogenic factors, growth factor receptors and amplicons as well as bacterial and SAg plasmids and DNA are prepared as described in the text references. When necessary, they are modified to forms suitable for transfection into mammalian tumor cells or accessory cells using methods well described in the art. (Old R W et al, Principles of Gene Manipulation, 5th Ed., Blackwell 1994).

As a representative SAg, enterotoxin B plasmid DNA is prepared by the method of Jones C L et al., J. Bacteriology 166 29-33 (1986) and Ranelli et al., Proc. Natl. Acad Sci. USA 82:5850-5854 (1985) using the CsCl-ethidium bromide density gradient centrifugation of cleared lysates as described (Clewell, D B et al., Proc. Natl. Acad. Sci. USA 62-1159-1166 (1969)). S. aureus chromosomal DNA was isolated as described by Betley M et al., Proc. Natl. Acad. Sci. USA 81: 5179-5183 (1984). E. coli HB101 was transformed with plasmid DNA by the CaCl2 procedure of Morrison D A et al., Meth. EnzymoL 68:326-331 (1979). Restriction digests were analyzed by 1% agarose and 5% acrylamide gel electrophoresis using Tris/Borate/EDTA buffer as described in Greene P J et al., Methods Mol. Biol. 7: 87-111 (1974). Additional methods for isolation and cloning of specific bacterial and mammalian plasmid DNA useful in tumor or accessory cell transfection are cited in references given previously in the text or in Snyder L et al., Molecular Genetics of Bacteria, ASM Press, Washington, D.C.(1997); Peters et al., supra; Franks et al., supra.

Suitable template DNA for production of nRNA encoding a desired polypeptide may be prepared using standard recombinant DNA methodology as described in Ausubel F et al. Short Protocols in Molecular Biology 3rd Ed. John Wiley, New York, N.Y. (1995). There are numerous available cloning vectors and any cDNA containing an initiation codon can be introduced into the selected plasmid and mRNA can be prepared from the resulting template DNA. The plasmid can be cut with an appropriate restriction enzyme to insert any desired cDNA coding for a polypeptide of interest. For example the readily available cloning vector pSP64T can be used after linearization and transcription with SP6 RNA polymerase. Smaller sequence may be inserted into the Hind III/EcoTI fragment with T4 ligase. Resulting plasmids are screened for orientation and transformed into E. coli. These plasmids are adapted to receive any gene of interest at a unique BgIII restriction site which is placed between the two Xenopus b-globin sequences.

Subcloning of SEB into pHβ-Apr-1-Neo Expression Vector

The Staphylococcal enterotoxin B (SEB) gene has been subcloned into pHβ-Apr-1-neo expression vector. The final construct contained only the coding sequence of SEB and conferred resistance to ampicillin and G-418.

Materials and Methods

PCR:

1.The following two primers are designed and made at Life Technologies, Inc.:

Primer SEB1: total 24 bp (SEQ ID NO: 3) 5′ to 3′ GGC.GTC.GAC.ATG.TAT.AAG.AGA.TTA

Sa/I Site

Primer SEB2: total 24 bp (SEQ ID NO: 4) 5′ to 3′ GCC.GGA.TCC.TCA.CTT.TTT.CTT.TGT

BamHI Site

Both primers were dissolved in filter-sterilized ddH20 to a final concentration of 20 mM (stock solution).

2.The volume (in ml) of reagents for each PCR reaction is listed below:

| Reagent | Exp. 1 | Exp. 2 | Exp. 3 | Exp. 4 | Exp. 5 |
|---|---|---|---|---|---|
| ddH2O | 76 | 72 | 67 | 49 | 59 |
| 10 X PCR buffer | 10 | 10 | 10 | 10 | 10 |
| 10 X dNTP | 10 | 10 | 10 | 10 | 10 |
| (2 mM stock) | | | | | |
| Primer SEB1 | 1 | 5 | 1 | 10 | 10 |
| (20 mM stock) | | | | | |
| Primer SEB2 | 1 | 1 | 1 | 10 | 10 |
| (20 mM stock) | | | | | |
| SEB Template | 1 | 1 | 10 | 10 | 0 |
| (50 mg stock) | | | | | |
| Pfu Turbo Enz | 1 | 1 | 1 | 1 | 1 |
| Final Volume | 100 | 100 | 100 | 100 | 100 |

3.The following cycling parameters were applied:

95° C. 1 minute 1 cycle initial denature

95° C. 45 seconds denature

52° C. 1 minute 20 cycles anneal

72° C. 1 minute extension

72° C. 1 minute 1 cycle final extension

4° C. hold

4.To verify that the PCR reactions yielded the correct size fragment, 10 ml of the reaction mixture was electrophoresed on a 1% agarose gel in 1×TAE buffer.

Vector

1. The pHβ-Apr-1-neo expression vector was spotted the vector on a filter paper. See FIG. 1

2. To recover the DNA, the circle was cut out and added to 100 ml of H2O to allow rehydration for 5 minutes. After a brief centrifugation, the supernatant was used to transform E. coli XL1Blue (Stratagene), and selected by ampiciuin (final concentration 100 mg/ml).

3. To verify that the vector is correct, 4 ampR clones were randomly selected and the clones were cultured in LB amp media. DNA was isolated and digested with SalI, BamHI (single digest) and EcoRI/HindIII (double digest). The digested products were electrophoresed on a 1% agarose gel in 1×TAE buffer. The profile of the restriction digest confirmed that the vector is correct.

Cloning and Verification

1. The correct PCR fragments in experiments 2, 3, and 4 were pooled and gel-purified. A portion of the fragments was digested with restriction enzymes SalI and BamHI, and was ligated into the digested pHβ-Apr-1-neo expression vector. The ligation products were transformed into E. coli XL1Blue (Stratagene). Insert containing clones were selected by ampicillin.

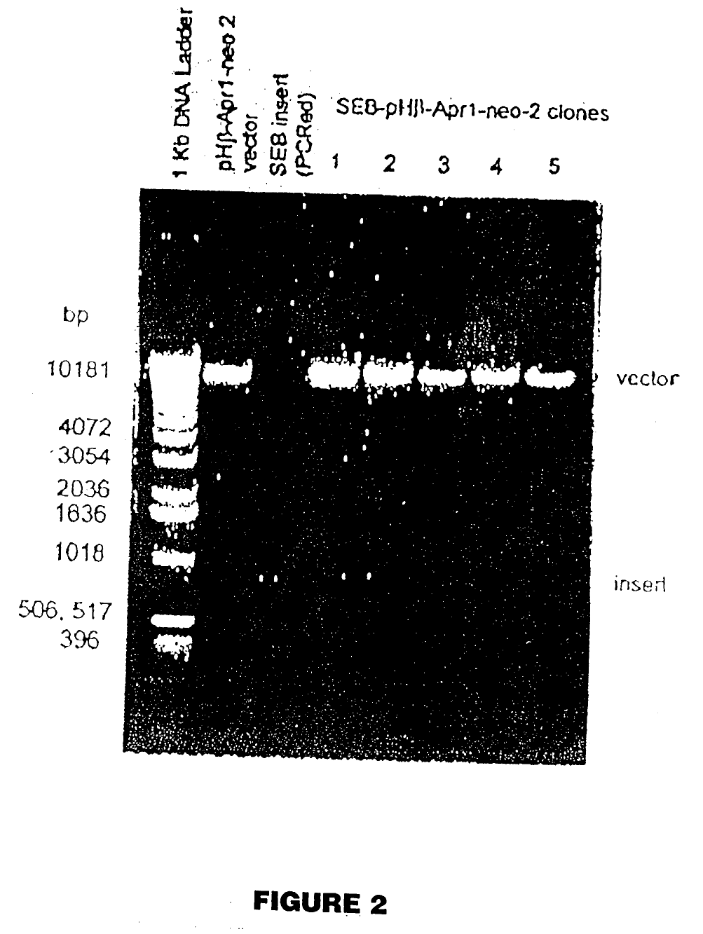

2. Ten ampicillin resistant clones were randomly selected, cultured in 5 ml of LB amp media, and their plasmid DNA was isolated. Insert containing clones (SEB construct) were verified by digesting the DNA with SalI and BamHI restriction endonucleases and electrophoresis at 0.8% agarose gel. (FIG. 2)

3. One of the SEB constructs (clone #2) was verified by sequencing and aligned with the published SEB sequence (FIG. 3).

Purified DNA templates from bacteria and human cells are prepared for introduction of plasmid into human and bacterial cells by additional methods given in Ausubel F et al., supra. The plasmid DNA is grown up in E. coli in ampicillin containing LB medium

The cells were then pelleted by spining a 5000 rpm for 10 min. at 5000 rpm., resuspended in cold TE pH 8.0, centrifuged again for 10 minutes. at 5000 rpm., resuspended in a solution of 50 mM glucose, 25 mM Tris-Cl pH 8.0, 10 mM EDTA and 40 mg/ml lysozyme. After incubation for 5-10 min. with occasional inversion, 0.2 N NaOH containing 1% SDS was added, followed after 10 minutes at 0° C. with 3 M potassium acetate and 2 M acetic acid. After 10 more minutes, the material was again centrifuged a 6000 rpm, and the supernatant was removed with a pipet. The pellet was then mixed into 0.6 vol. isopropanol (−20° C.), mixed, and stored at −20° C. for 15 minutes. The material was then centrifuged again at 10,000 rpm for 20 min., this time in an HB4 singing bucket rotor apparatus after which the supernatant was removed and the pellet was washed in 70% EtOH and dried at room temperature. Next, the pellet was resuspended in 3.5 ml TE, followed by addition of 3.4 g CsCl and 350 ml of 5 mg/ml EtBr. The resulting material was placed in a quick seal tube, filled to the top with mineral oil. The tube was spun for 3.5 hours at 80,000 rpm in a VTi80 centrifuge. The band was removed and the material was centrifuged again making up the volume with 0.95 g CsCl/ml and 0.1 ml or 5 mg/ml EtBr/ml in TE. The EtBr was then extracted with an equal volume of TE saturated N-Butanol after adding 3 volumes of TE to the band. Next, 2.5 vol. EtOH was added, and the material was precipitated at −20° C. for 2 hours. The resultant DNA precipitate is used as a DNA template.

Transfection of B16F10 Melanoma Cells

G418 sensitivity: B16F10 melanoma cells (B16s) were first tested for sensitivity to G418 which will be used as the selectable marker. At 400 ug/mL G418, B16s did not survive, while 200 and 300 ug/mL allowed some survival.

Transfection:

Lipofectamine was used to produce stably transfected B16s. The conditions for transfection were those described protocol provided by Life Technologies. B16s were plated at 4×105 cells/well in 6 well plates, using Murine Complete Medium (MCM) described in Report 2. Cells were cultured overnight. Optimal density is 50-80% confluent and is usually achieved by 18-24 after seeding at 1-3×10⁵ cells/well. DNA sources consisted of SEB-G418 resistance containing vector, vector DNA with G418 resistance gene only, and control DNA from PSK401 (no G418 resistance marker). DNA concentrations were determined for the SEB containing and control vectors.

| DNA source | A260 | DNA (ug/ml) |
|---|---|---|
| SEB | 0.09 | 0.45 |
| Vector only | 0.13 | 0.65 |
| PSK 401 | 0.15 | 0.75 |

Lipofectamine solutions and DNA solutions were prepared in 12×75 mm tubes, using OPTI-MEM (Life Technologies 31985). DNA solutions contained approximately 2 ug in 100 μL OPTI-MEM; the LIPOFECTAMINE Reagent was diluted by adding 6 or 12 uL to OPTI-MEM at a final volume of 100 uL. The solutions were mixed and held at room temperature for 30 minutes. Specific DNA and Lipofectamine conditions were as follows:

Plated cells were rinsed once with 2 ml/well OPTI-MEM. To the above tubes, 0.8 mL OPTI-MEM. This mixture was then overlayed onto the washed cell monolayers according to the above well designations. Cells were incubated for 5 hours at 37° C. in 5% CO2. Murine Complete Medium with 20% FBS but no antibiotics was then added at 1 ml/well. Cultures were refed with standard MCM, at 3 mL/well, after 24 hours. Three days after transfection, cells from each transfection condition were subcultured by splitting the total cell suspension 90:10 into 150 mm plates (one plate received 90% of the cell suspension, the other received the remaining 10%).

G418 Selection

All plates were refed at 6 days after transfection with medium containing 400 ug/ml G418. Plates were refed every 2 to 3 days with G418 containing medium until day 17 after transfection. No growth was observed in wells 1-4 as expected. Plates initiated with 90% of the cell suspension and showing growth were harvested, frozen, and stored at −80° C.

Primary Subcloning

Ten colonies were selected from each well for wells 5, 7, 9, and 11. Subcloning was accomplished by the use of cloning cylinders as follows: After seating the cylinder, medium was aspirated and the isolated colony was washed once with 100 ul of warmed trypsin-EDTA. This was aspirated and replaced with fresh tyrpsin-EDTA. After incubation at 37° C. for 2 minutes, the cells were recovered by trituration and transferred to a tube containing 1 ml MCM, then replated by addition of 20 ul of cell suspension to 15 mL MCM with G418 in 150 mm plates. The remaining cell suspension was plated into 24 well plates, 4 wells/clone and all plates were maintained at 37° C., 5% CO₂. The 6 well plates were used to assess SEB expression on the cell surface as desribed under Detection of positive clones.

Secondary and Tertiary Subcloning and Preparation of Frozen Stocks

These and all subsequent procedures were performed by me. Secondary subdloning was performed as above at 7 days after initiation of primary subclones. One colony/plate was selected for further subcloning (a total of 40 colonies) The cell suspension was prepared in a total volume of 1 mL; 100 ul was replated into 100 mm plates containing 10 ml MCM with G418. The remaining cell suspension was plated in 96 well plates at 100/well, 2 replicates for assay. The 96 well plate was used for detection of intracellular expression of SEB desribed under Detection of positive clones.

Primary subcloning plates were cultured one additional day, then harvested, frozen, and stored at −80° C. These frozen stocks are designated primary subclones. Secondary subclones were refed after 4 days. Of 40 secondary clones, 36 regrew. Tertiary subcloning was performed after 8 days and frozen stocks of secondary clones were prepared after 9 days. Tertiary clones were refed after 3 days in culture and subcultured after 7 days in culture. Plates were harvested, cells were resuspended in a total of 1 mL, and replated by addition of 100 ul of the cell suspension to 100 mm plates with 15 ml MCM or 100 u/well in a 96 well plate. Frozen stocks of tertiary clones were prepared.

Generation of Conditioned Medium for Assay of Supernatents

After 7 days, 100 mm plates of tertiary clones were again replated. This time, cell counts were performed and 4.5×10⁵ cells were plated in 12 well plates, one well/clone. The remaining cell suspension was frozen and stored at −80° C. After 4 days in culture, supematents were harvested, stored at 4° C., and the cells were replated into 100 mm plates. Supematents were obtained from the 100 mm plates after 7 days in culture. See Detection of positive clones. Frozen stocks were also generated from these plates.

Development of ELISA with HRP Rabbit Anti-SEB.

Final ELISA conditions were as follows:

| | |
|---|---|
| Assay Plate | Pro Bind (Falcon # 3915) |
| Capture | Rabbit anti-SEB (Toxin Technologies # LBI202), 10 |
| Antibody | in PBS, 50 μL/well, 1 hr, RT |
| ug/mL | |
| Wash | 3X with 0.1% casein, 0.1% Tween 20 in PBS |
| Blocking | 1% casein in PBS, 250 μL/well, overnight, 4oC |
| Antigen | Supernatant used neat or SEB diluted in PBS, 50 |
| | μl/well, 2 hr, RT |
| Wash | As above |
| Primary Ab | HRP Rabbit anti-SEB (Toxin Technologies # LBC202), |
| | 1/300 in block buffer, 50 μL/well, 2 hr, RT |
| Substrate | OPD, 2.5 mg/ml in citrate buffer, pH 5.0, 0.03% H₂O₂, |
| | 100 μl/well, 15 min, RT |
| Stop | 4 M H₂SO₄, 100 μl/well |
| Read-out | OD 490 nm |

Results: SEB produced a dose response curve (linear range 60 fg-60 pg/mL) and the background was very low. Vector only clones produced only background signals. One SEB transfected clone produced a strong signal, three produced moderate signals, and one other produced a weak but definite signal.

OD 490 nm

| | SEB+ | | | Vector only | | |
|---|---|---|---|---|---|---|
| | 1 | 2 | mean | 1 | 2 | mean |
| 9.1 | 0.097 | 0.112 | 0.104 | 0.079 | 0.102 | 0.091 |
| 9.2 | 0.127 | 0.123 | 0.125 | 0.081 | 0.076 | 0.078 |
| 9.3 | 0.109 | 0.104 | 0.106 | 0.087 | 0.070 | 0.079 |
| 9.4 | 0.444 | 0.393 | 0.418 | 0.077 | 0.077 | 0.077 |
| 9.5 | 0.163 | 0.087 | 0.125 | 0.075 | 0.074 | 0.074 |
| 9.6 | 0.516 | 0.522 | 0.519 | 0.066 | 0.064 | 0.065 |
| 9.7 | 0.087 | 0.091 | 0.089 | 0.096 | 0.084 | 0.090 |
| 9.8 | 0.386 | 0.450 | 0.418 | 0.080 | 0.071 | 0.075 |
| 9.9 | 0.137 | 0.122 | 0.130 | 0.071 | 0.070 | 0.071 |
| 11.1 | 0.083 | 0.075 | 0.079 | 0.068 | 0.078 | 0.073 |
| 11.2 | 1.847 | 1.802 | 1.824 | 0.063 | 0.076 | 0.070 |
| 11.3 | 0.071 | 0.077 | 0.074 | 0.076 | 0.074 | 0.075 |
| 11.4 | 0.087 | 0.084 | 0.086 | 0.083 | 0.085 | 0.084 |
| 11.5 | 0.161 | 0.220 | 0.191 | 0.092 | 0.086 | 0.089 |
| 11.8 | 0.221 | 0.100 | 0.160 | 0.080 | 0.081 | 0.080 |
| 11.9 | 0.080 | 0.091 | 0.085 | 0.077 | 0.072 | 0.074 |
| 11.10 | 0.290 | 0.254 | 0.272 | 0.081 | 0.112 | 0.097 |
| 11.10 | 0.268 | 0.263 | 0.265 | 0.093 | 0.114 | 0.103 |

Based on the the SEB standard curve, the following concentrations were derived.

| Clone number (pg/ml) | SEB |
|---|---|
| 11.2 | 4.146 |
| 9.6 | 0.152 |
| 9.4 | 0.118 |
| 9.8 | 0.118 |
| 11.10 | 0.081 |

Cells are transfected ex vivo or in vivo and implanted in a cancer-bearing host. These transfected cells are also used to stimulate host lymphocytes ex vivo. Once activated, the lymphocytes are administered to the host. The ex vivo or in vitro introduction of DNA into cells is accomplished by methods that (1) form DNA precipitates which are internalized by the target cell; (2) create DNA-containing complexes with charge characteristics that are compatible with DNA uptake by a target cell; or (3) result in the transient formation of pores in the plasma membrane of a target cell exposed to an electric pulse (these pores are of sufficient size to allow DNA to enter the target cell).

Generally, two factors determine the method used: the duration of expression required (i.e., transient versus stable expression) and the type of cell to be transfected. The specific details of exemplary procedures are described herein. Transitions are carried out by well established methods including calcium phosphate precipitations, DEAE Dextran transfection, and electroporation.

Calcium Phosphate Precipitation

A commonly used ex vivo and in vitro method to transfer DNA into recipient cells involves the co-precipitation of the DNA of interest with calcium phosphate. With this technique, DNA enters the cell in sufficient quantities such that the treated cells are transformed with relatively high frequency. Using a variety of cell types, transfection efficiencies of up to 10-3 have been obtained. This is the method of choice for the generation of stable transfectants.

Variations of the basic technique have been developed. If the transfection involves the transfer of plasmid DNA, then high molecular weight genomic DNA isolated from a defined cell or tissue source can be included. The addition of such DNA, called carrier DNA, often increases the efficiency of transfection by the plasmid DNA. Upon arrival of the plasmid DNA/carrier DNA/calcium phosphate co-precipitate to the nucleus of the treated cell, the plasmid DNA integrates into the carrier DNA, often in the tandem array, and this assembly of plasmid and carrier DNA, called a transgenome, subsequently integrates into the chromosome of the host cell.

Another procedural option is the addition of a chemical shock step to the transfection protocol. Either dimethylsulfoxide or glycerol are appropriate. The optimal concentrations and lengths of treatment vary according to cell type. The use of these agents dramatically affect cell viability and can be optimized as described elsewhere [Chen and Okayama, Mol. Cell. Biol. 7:2745 (1987)]. Specifically, incubation of cells with the co-precipitate is optimal at 35° C. in 2-4% CO2 for 15-24 hours. In addition, circular DNA is more active than linear DNA and a finer precipitate is obtained when the DNA concentration is between 20-30 mg/ml in the precipitation mix.

It is noted that incubator temperature, CO2 concentration, and DNA concentration can be varied to obtain the desired result. In addition, the temperature and CO2 concentrations described below are not optimal for cell growth and should be maintained only temporarily.

Method

Day 1: 1.3×10⁶ cells are seeded per 100-mm dish. Cells are about 75% confluent when used to seed the dishes.

Day 2: A large calcium phosphate cocktail mixture to transfect many plates simultaneously is prepared. This protocol is given for 1 ml (or 1×100-mm dish equivalent) of solution. These amounts are scaled up as necessary, allowing for an appropriate amount of sample-transfer errors. Adherence to sterile technique is critical. Sterile reagents, tips, and tubes are used.

1. Add 1-20 g DNA (1 mg/ml in sterile TE, 10 mM Tris-HCl 1 mM EDTA pH 7.05) to 0.45 ml sterile H2O. Note: First “sterilize” DNA by ethanol precipitation with NaCl (0.1 M final aqueous concentration) and 2×volume 200% ethanol.

2. Add 0.5 ml 2×HEPES buffered saline. Mix well.

3. Add 50 ml of 2.5 M CaCl₂, vortex immediately.

4. Allow the DNA mixture to sit undisturbed for 15-30 minutes at room temperature.

5. Add 1 ml ofthe DNA transfection cocktail directly to the medium in the 100-mm dish (plated with cells on day 1).

6. Incubate the dishes containing the DNA precipitate for 16 hours at 37° C. Remove the media containing the precipitate and add fresh complete growth media.

7. Allow the cells to incubate for 24 hours. Post-incubation, the cultures can be split for subsequent selection. Split cultures 1:5; however, to isolate individual colonies for further analysis, split cultures 1:10 and 1:100.

DEAE Dextran Transfection

Typically, DEAE dextran transfection is used to transiently transfect cells in culture. This method is highly efficient and the DNA/DEAE dextran mixture used for transfection is relatively easy to prepare. For example, this method yields transfection efficiencies of as high as 80 percent. DNA introduced into cells with this method, however, appears to undergo mutations at a higher rate than that observed with calcium phosphate-mediated transfection.

Method

Briefly, a DEAE dextran mixture is prepared and the DNA sample of interest is added, mixed, and then transferred to the cells in culture.

Day 1: Cells are seeded at a concentration of 2×10⁴ cells/cm2 in a total volume of 2 ml/well (1.92×10⁵ cells/well of a six-well cluster dish). Cells should be about 75% confluent when used to seed the dishes.

Day 2: Resuspend 0.5 ml DEAE Dextran in Tris-buffered saline (TBS).

Final DEAE Dextran concentration should be about 0.04%. Observe cell monolayers microscopically. Cells should appear about 60-70% confluent and well distributed. Bring all reagents to room temperature. Aspirate off growth media and wash monolayer once with 3 ml of phosphate buffered saline (PBS), followed by one wash with 3 ml of TBS. Aspirate off TBS solution and add 100-125 ml of the appropriate DNA/DEAE-Dextran/TBS mixture to the wells. Incubate dishes at room temperature inside a laminar flow hood. Rock the dishes every 5 minutes for 1 hour, making sure the DNA solution covers the cells. After the 1-hour incubation period, aspirate off the DNA solution and wash once with 3 ml of TBS followed by 3 ml of PBS. Remove the PBS solution by aspiration and replace with 2 ml of complete growth media containing 100 M chloroquine. Incubate the dishes in an incubator set at 37° C. and 5% CO2 for 4 hours. Remove the media containing chloroquine and replace with 2-3 ml of complete growth media (no chloroquine). Incubate the transfected cells for 1-3 days, after which the cells will be ready for analysis. The exact incubation period depends on the intent of the transfection. Optimal expression typically occurs at 3days post-transfection.

Electroporation

Electroporation is a process whereby cells in suspension are mixed with the DNA to be transferred. This cell/DNA mixture is subsequently exposed to a high-voltage electric field. This creates pores in the membranes of treated cells that are large enough to allow the passage of macromolecules such as DNA into the cells. Such DNA molecules are ultimately transported to the nucleus and a subset of these molecules are integrated into the host genome. The reclosing of the membrane pores is both time and temperature dependent and thus is delayed by incubation at 0° C., thereby increasing the probability that the molecule of interest will enter the cell.

Electroporation appears to work on virtually every cell type. With this technique, the efficiency of nucleic acid transfer is high for both transient transfection and stable transfection. One important technical difference between electroporation and other competing technologies is that the number of input cells required for electroporation is considerably higher.

Method

1. Harvest exponentially growing cells such as tumor cells or accessory cells by trypsinization, pellet, and wash twice with electroporation buffer (Kriegler, M. Gene Transfer and Expression, W. H. Freeman and Co., New York, N.Y. (1991)).

2. Resuspend cells in electroporation buffer at a concentration of 2-20×10⁶ cells/ml in an electroporation cuvette.

3. Add 5-25 mg of DNA that has been linearized to the cell suspension

4. Insert or connect the electroporation electrode according to the manufacturer's instructions and subject cell/DNA mixture to an electric field (pulse).

5. Return cell/DNA mixture to ice and incubate for 5 minutes.

6. Plate cells in non-selective medium Biochemical selection may be carried out 24-48 hours later.

Lipofectamine

In vitro cell transfections can be done in 12-well plates, using 3.0 g plasmid DNA and Lipofectamine (GIBCO BRL), at 37° C. for 4 hours. After transfection, the cells are cultured in 2.0 ml complete medium for 48 hours and the cells are harvested. The cells are then washed in PBS. Stably transfected Chinese hamster ovary (CHO) and B16 lines are isolated by selection in 1.0 mg/ml G418 (GIBCO BRL). Cells are grown and passaged in medium containing G418 for 3-4 weeks Mock transfected cell lines (cells transfected with vector only) are used as controls.

Viral Vectors

Recombinant viral vectors containing the nucleic acid of interest can also be used to introduce nucleic acid into a cell ex vivo or in vitro. It is noted that viral vectors are also used to transfect cells in vivo. These viral vectors can be DNA viruses such as herpesviruses, adenoviruses, and vaccinia viruses or RNA viruses such as retroviruses. The method and materials required to produce and use these viral vectors ex vivo, in vitro, and in vivo are commonly known in the art and are used in the invention described herein (Sambrook, J.et al., supra).

Selection

Regardless of the method used to transfect a particular cell type, stably transfected cells are identified as follows. The DNA of interest contains a selectable marker. Typically, a selectable marker encodes a polypeptide that confers drug resistance and the DNA containing this resistance conferring nucleic acid is transfected into the recipient cell. Post transfection, the treated cells are allowed to grow for a period of time (24-48) hours to allow for efficient expression of the selectable marker. After an appropriate incubation time, transfected cells are treated with media containing the concentration of drug appropriate for the selective survival and expansion of the transfected and now drug resistant cells.

Many drug as well as non-drug selection methods are known in the art and can be used in the invention described herein. For example, a detailed description of currently available drug selection strategies is provided in Kriegler M., Gene Transfer and Expression, A Laboratory Manual, W. H. Freeman and Co. New York, N.Y. pp.103-107 (1991).

General Method

Sixteen hours after transfection, the transfected/infected cells are fed with fresh, non-selective media. Twenty-four to forty-eight hours later, the cultures are split to a 1:5 or greater dilution and plated in drug-containing media. It is noted that cells are not placed in drug-containing media immediately after transfection in order to allow a sufficient amount of time for the drug resistance nucleic acid to be expressed and thus confer the drug resistant phenotype. Cell cultures are re-fed with drug-containing media every three days, at which time cultures are examined under a microscope to determine the efficiency of drug selection.

Site-Directed Mutagenesis by Polymerase Chain Reaction:

Introduction of Restriction Endonuclease Sites by PCR

PCR is the preferred method for introducing any desired sequence change into the DNA. The basic protocol is as follows:

Materials

DNA sample to be mutagenized, pUC19 plasmid b vector or similar high-copy number plasmid having M13 flanking primer.

500 ng/ml (100 pM/ml) flanking sequence primers incorporating the restriction enzyme site.

TE buffer

10× amplification buffer

2 mM 4dNTP mix

500 ng/ml (100 pM/ml) M13 flanking sequence primers: forward (NEB) and reverse (NEB)

5 U/ml Taq DNA polymerase

Buffered phenol

100% ethanol

Appropriate restriction endonucleases

500ml microcentrifuge tube

Automated thermal cycler

1. Subclone DNA to be mutagenized into high-copy number vector using restriction sites flanking the area to be mutated.

2. Prepare template DNA by plasmid miniprep. Resuspend 100 ng in TE buffer to 1 ng/ml final.

3. Synthesize oligonucleotide primers and purify by denaturing polyacrylaride gel electrophoresis. Resuspend oligonucleotides in 500 1 TE buffer. Determine absorbance at A260 and adjust to 500 ng/ml.

4. Combine the following in each of two 500 1 microcentrifuge tubes, adding oligonucleotides 1 and 2 to separate tubes:

10 ml (10 ng) template DNA

10 ml 10× amplification buffer

10 ml 2 mM 4dNTP mix

1 ml (500 ng) oligonucleotide 1 or 2 (100 pM final)

1 ml (500 ng) appropriate M 13 flanking sequence primer, forward or reverse (100 pM final).

H₂O to 99.5 ml

0.5 ml Taq DNA polymerase (5 U/ml)

Overlay reaction with 100 ml mineral oil.

5. Carry out PCR in an automated thermal cycler for 20 to 25 cycles under the following conditions:

45 sec 93° C.

2 min 50° C.

2 min 72° C.

After last cycle, extend for an additional 10 min at 72° C.

6. Analyze 4 1 by nondenaturing agarose or occurrence gel electrophoresis to verify that the amplification has yielded the predicted product.

7. Remove mineral oil and extract once with chloroform to remove remaining oil. Extract with buffered phenol and concentrate by precipitation with 100% ethanol.

8. Digest half the amplified DNA with the restriction endonucleases for the flanking and introduced sites. Purify digested fragments on a low gelling/melting agarose gel.

9. Ligate and subclone both fragments into an appropriately digested vector to obtain a recombinant plasmid containing a single DNA fragment incorporating the new restriction site.

10. Transform plasmid into E. coli. Prepare DNA by plasmid miniprep.

11. Analyze amplified fragment portion of plasmid by DNA sequencing to confirm the addition of the mutation.

Introduction of Point Mutation by PCR:

Materials

DNA sample to be mutagenized

Oligonucleotide primers incorporating the point mutation

Klenow fragment of E. coli DNA polymerase I

Appropriate restriction endonuclease

Procedure

1. Prepare template DNA (steps 1 and 2 of Basic Protocol).

2. Synthesize and purify oligonucleotide primers (3 and 4).

3. Amplify template DNA (steps 4 and 5 of Basic Protocol 1). After final extension step, add 5 U Klenow fragment and incubate 15 min at 30° C.).

4. Analyze and process reaction (steps 6 and 7 of Basic Protocol).

5. Digest half the amplified fragments with the restriction endonucleases for the flanking sequences. Purify digested fragments on a low gelling/melting agarose gel.

6. Subclone the two amplified fragments into an appropriately digested vector by blunt-end ligation.

7. Carry out steps 10 and 11 of Basic Protocol.

Introduction of a Point Mutation by Sequential PCR

Steps

1. Prepare the template DNA (steps 1 and 2 of Basic Protocol 1).

2. Synthesize and purify the oligosaccharide primers (5 and 6).

3. Amplity the template and generate blunt-end fragments (step 3 of Basic Protocol).

4. Purify fragments by nondenaturing agarose gel electrophoresis. Resuspend in TE buffer at 1 ng/ml.

5. Combine the following in 500 ml microcentrifuge tube:

10 ml (10 ng) each amplified fragment

1 ml ( 500 ng) each flanking sequence primer (each 100 pM final)

10 ml 10× amplification buffer

10 ml 2 mM 4dNTP mix

0.5 ml Taq DNA polymerase (5 U/ml)

Overlay with 100 ml mineral oil.

6. Carry out PCR for 20 to 25 cycles (step 5 of Basic Protocol 1). Analyze and process the reaction mix (steps 6 and 7 of Basic Protocol 1).

7. Digest cDNA fragment with appropriate restriction endonuclease for the flanking sites. Purify fragment on a low gelling/melting agarose gel. Subclone into an appropriately digested vector.

8. Carry out steps 10 and 11, Basic Protocol 1.

Genomic Targeting and Genetic Conversion in Cancer Therapy

A number of cellular transformations are due, in large part, to a single base mutation that alters the function of the expressed protein. Alterations in the DNA sequence of a gene involved in cell proliferation can have a significant effect on the viability of particular cells. Thus, the capacity to modulate the base sequence of such a gene would be a useful tool for cancer therapeutics. An experimental strategy that centers around site-specific DNA base mutation or correction using a unique chimeric oligonucleotide has been developed. This chimeric molecule has demonstrated higher recombinogenic activities than identical oligonucleotides containing only DNA residues, both in vitro and in vivo. The chimeric molecule is designed to hybridize to a target site within the genome and induce a single base mismatch at the residue targeted for mutation. The DNA structure created at this site is recognized by the host cell's repair system which mediates the correction reaction. For example, the bcr-abl fusion gene, the product of a translocation between human chromosomes 9 and 22, and the cause of chronic myelogenous leukemia (CML) can be targeted for gene correction. Fusion genes or mutations which abound in cancer cells are excellent targets for correction especially if (1) they are unique and are recognized by the immune system as dominant or subdominant epitopes, (2) they are a single copy target; (3) the DNA sequence of the fusion gene or mutation is unique. The goal of such experiments is to knock-out the fusion gene by changing an amino acid codon into a stop codon through a chimeric directed DNA repair system.

Targeted Gene Correction of Episomal DNA in Mammalian Cells Mediated by a Chimeric RNA/DNA Oliponucleotide

An experimental strategy to facilitate correction of single-base mutations of episomal targets in mammalian cells has been developed. The method utilizes a chimeric oligonucleotide composed of a contiguous stretch of RNA and DNA residues in a duplex conformation with double hairpin caps on the ends. The RNA/DNA sequence is designed to align with the sequence of the mutant locus and to contain the desired nucleotide change. Activity of the chimeric molecule in targeted correction is used in a with the aim of correcting a point mutation in the gene encoding the human liver/bone/kidney alkaline phosphatase. When the chimeric molecule is introduced into cells containing the mutant gene on an extrachromosomal plasmid, correction of the point mutation is accomplished with a frequency approaching 30%. These results extend the usefulness of the oligonucleotide-based gene targeting approaches by increasing specific targeting frequency.

The site directed mutagenesis is used to carry out using the chimeric DNA/RNA structure which enables the construct to target tumor cells in vivo and in vitro. Such targeting structures include target seeking moieties and can in principle be any structure that is able to bind to a cell surface structure or that binds via biospecific affinity. The target seeking moiety is primarily a disease specific structure selected among hormones, antibodies, growth factors. The biospecific affinity counterpart may include interleukins (especially interleukin-2) antibodies (full length antibody, Fab, F(ab′2, Fv, single chain antibody and any other antigen binding antibody fragments (such as Fab) directed to a cells surface epitope or more preferably towards the binding epitope for the a specific antibody. They may also include polypeptides binding to the constant domains of immunoglobulins (e.g., protein A and G and L), lectins, streptavidin, biotin etc. The term antibodies comprises monoclonal as well as polyclonal preparations. The targeting moiety may also be directed toward unique structures on more or less healthy cells that regulate or control the development of a disease, or ligands for specific receptors on tumor cells). The targeting structure may be a nucleic acid, lipid or carbohydrate and variations thereof which target receptors on the diseased cell. The targeting is not confined to diseased cells but may include additional normal cells as well.

Synthesis and Purification of Oligonucleotides

The chimeric oligonucleotides are synthesized on a 0.2-mol scale by using the 1000 Å-wide-pore CPG on the ABI 394 DNA/RNA synthesizer. The exocyclic amine groups of DNA phosphoramidites (Applied Biosystems) are protected with benzoyl for adenosine and cytidine and isobutyryl for guanosine. The 2′-O-methyl RNA phosphoramidites (Glen Research, Sterling, Va.) are protected with a phenoxyacetyl group for adenosine, dimethylformamide for guanosine and an isobutyryl group for cytidine. After the synthesis is complete, the base-protecting groups are removed by heating in ethanol/concentrated ammonium hydroxide, 1:3 (vol/vol), for 20 h at 55° C. The crude oligonucleotides are purified by polyacrylamide gel electrophoresis. The entire oligonucleotide sample is mixed with 7 M urea/10% (vol/vol) glycerol. heated to 70° C., and loaded on a 10% polyacrylarnide gel containing 7 M urea. After gel electrophoresis, DNA is visualized by UV shadowing, dissected from the gel, crushed, and eluted overnight in TE buffer (10 mM Tris-HCl/1 mM EDTA, pH 7.5) with shaking. The eluent containing gel pieces are centrifuged through 0.45-um (pore size) spin filter (Millipore) and precipitated with ethanol. Samples are further desalted with a G-25 spin column (Boerhinger Mannheim) and greater than 95% of the purified oligonucleotides are found to be full length.

Transient Transfection and Measurements of Activity

CHO cells were maintained in Dulbecco's modified Eagle's medium (DMEM) (BRL) containing 10% (vol/vol) fetal bovine serum (FBS; BRL). Transient transfection is carried out by addition of 10 g of the plasmid with 10 g of Lipofectin in 1 ml of Optimem

(BRL) to 2×10⁵CHO cells in a 6-well plate. After 6 h. various amounts of oligonucleotide is mixed with 10 g of Lipofectin in 1 ml of Optimem and added to each well. After 18 h, the medium is aspirated and 2 ml of DMEM containing 10% FBS was added to each well. Histochemical staining was carried out (19), 24 h after transfection of the oligonucleotide. Spectrophotometric measurements are carried out by the ELISA amplification system (BRL). Transfection is carried out in triplicate in a 96-well plate. The amounts of reagents and cells are 10% of that used for the 6-well plate. Cells were washed three times with 0.15M NaCl and lysed in 100 ml of buffer containing 10 mM NaCl, 0.5 Nonidet P-40, 3 mM MgCl2, and 10 mM Tris-HCl (pH 7.5), 24 h after transfection with chimeric olgonucleotides. A fraction of cell lysates (20 ml) incubated with 50 1 of ELISA substrate and 50ml of ELISA amplifier (BRL), the reaction is stopped by addition of 50 ml of 0.3 M H2S04 after 5 min of incubation with amplifier. The extent of reaction is carried out within the linear range of the detection method. The absorbance is read by an ELISA plate reader (BRL) at a wavelength of 490 nm.

Hirt DNA Isolation, Colony Hybridization, and Direct DNA Sequencing of PCR Fragments

The cells are harvested for vector DNA isolation by a modified alkaline lysis procedure, 24 h after transfection with the chimeric oligonucleotide. Hirt DNA is transformed into Escherichia coli DH5a cells (BRL). Colonies from Hirt DNA are screened for specific hybridization for each probe designed to distinguish the point mutation. Colonies were grown on ampicillin plates, lifted onto nitrocellulose filter paper in duplicates, and processed for colony hybridization. The blots were hybridized to ³²P-end-labeled oligonucleotide probes at 37° C. in a solution containing 5×Denhardt's solution, 1% SDS, 2×SSC, and denatured salmon sperm DNA (100 mg/ml). Blots were washed at 52° C. in TMAC solution (3.0 M teramethylammonium chloride/50 mM Tris-HCl, pH 8.0/2 mM EDTA/0.1% SDS). Plasmid DNA was made from 20 colonies shown to hybridize to either of the probes by using the Qiagen miniprep kit (Chatsworth. Calif.). Several hundred bases flanking key positions of each plasmid are sequenced in both directions by automatic sequencing (ABI 373A, Applied Biosystems). A 192-bp PCR-amplified fragment are generated by Vent polymerase (New England Biolabs. Mass.), utilizing primers corresponding to positions of the known cDNA flanking position. The fragment is gel-purified and subjected to automatic DNA sequencing (ABI 373A, Applied Biosystems).

Oligonucleotide Synthesis

Chimeric RNA/DNA oligonucleotides for both transcribed and nontranscribed factor IX were synthesized by Applied Biosystems, Inc. (Foster City, Calif.) as previously described. The oligonucleotides are prepared with DNA and 2-O-methyl RNA phosphoraridite nucleoside monomers on an ABI 394 DNA/RNA synthesizer, purified by HPLC and quantified by UV absorbance. More than 95% of the purified oligonucleotides are determined to be full length.

Cell Isolation and Transfections

Cells are isolated, by a two-step collagenase perfusion as previously described. The purified cells are plated on Primaria plates (Becton Dickinson, Franklin Lakes, N.J.) at a density of 4×10⁶ cells per 35-mm dish and maintained in supplemented William's E medium. Eighteen hours after plating, the cells are washed and transfected with the chimeric molecules complexed to polyethylenimine (PEI). A pH 7.0, 10 mM stock solution of PEI (800 kDa) (Fluka Chemical Corp., Ronkonkoma, N.Y.) is prepared. Briefly, the chimeric oligonucleotides are complexed with 10 mM PEI at 9 equivalents of PEI nitrogen per chimeric phosphate in 100 1 of 0.15 M NaCl and transfected in 1 ml of medium at final concentrations of 150, 300 or 450 nM. PEI is lactosylated by coupling lactose to 30% of the nitrogen amines using sodium cyanoborobydride (Sigma Chemical Company, St. Louis, Mo.). Cells are also transfected 1 with 100 1 of 0.15 M NaCl containing the lactosylated 800-kDa and 25-kDa PEI chimeric complexes (Sigma) at final concentrations of 90, 180 or 270 nM. After 18 h, an additional 2 ml of medium is added to the transfected cultures for the remaining 6 or 30 h of incubation. Vehicle control transfections utilize the same amount of PEI, but substituted an equal volume of 10 mM Tris-HCl, pH 7.6, for the oligonucleotides.

DNA/RNA Isolation and Cloning

The cells were harvested by scraping 48 h after transfection. Genomic DNA larger than 100-150 base pairs was isolated using the highly pure PCR template preparation kit (Boehringer Mannheim, Indianapolis, Id.). RNA was isolated using RNAzoI 8 (Tel-Test, Inc., Friendswood, Tex.), according to the manufacturer's protocol. PCR amplification of a fragment of the gene in question gene is performed with 300 ng of the isolated DNA from either the primary cell culture.

The primers were designed (Oligos Etc., Wilsonville, Oreg.) corresponding to nucleotides to cDNA to be corrected (ref. 25). Primer annealing is carried out at 59° C., and the samples are amplified for 30 cycles using Expand Hi-fidelity polymerase (Boehringer Mannheim). To rule out PCR artifacts, 300 ng of control DNA is incubated with 0.5, 1.0 and 1.5 g of the oligonucleotide before the PCR-amplification reaction. Additionally, 1.0 g of the chimeric alone is used as the “template” for the PCR amplification.

RT-PCR amplification is done utilizing the Titian one tube RT-PCR system (Boehringer Mannheim) according to the manufacturer's protocol and by using the same primers as those used for the DNA PCR amplification. To rule out DNA contamination, the RNA samples are treated with RQ1 DNase-free RNase (Promega Corp., Madison, Wis.) and RT-PCR negative controls of RNased RNA samples were performed in parallel with the RT-PCR reaction. Each of the PCR reactions is ligated into the TA cloning vector pCR 2.1 (Invitrogen, San Diego, Calif.) and transformed into frozen competent E. coli.

Nuclear Uptake of the Chimeric Molecules Nuclear localization of fluorescently labeled chimeric oligonucleotides was determined in the isolated cells. For in vivo studies, 250 1 saline containing 75 g of fluorescently labeled chimeric oligonucleotides complexed to PEI is injected directly into the exposed caudate lobe. The animals are killed 24 h post injection, the tumor targeted is removed, bisected longitudinally, embedded using OCT and frozen cryosections were cut ˜10 pm thick, fixed, processed and examined using a MRC1000 confocal microscope (Bio-Rad, Inc., Hercules, Calif.).

In Vivo Delivery of the Chimeric Oligonucleotides

Vehicle controls and lactosylated 25-kDa PEI at a ratio of 6 equivalents of PEI nitrogen per chimeric phosphate are prepared in 300 1 of 5% dextrose. The aliquots are administered either as a single dose of 100 g or divided doses of 150 g and 200 g on consecutive days. Five days post injection, tumor tissue is removed for DNA and RNA isolation. DNA is isolated. RNA is isolated for RT-PCR amplification of the same region as the genomic DNA using RNAexol and RNAmate (Intermountain Scientific Corp., Kaysville, Utah) according to the manufacturer's protocol.

Colony Hybridization and Sequencing

Eighteen to 20 h after plating, the colonies were lifted onto MSI MagnaGraph nylon filters (Micron Separations, Inc., Westboro, Mass.), replicated and processed for hybridization according to the manufacturer's recommendation. The filters were hybridized for 24 h with 32P-end-labeled oligonucleotide probes (Life Technologies, Inc., Gaithersburg, Md.), where the underlined nucleotide is the target of mutagenesis. Hybridizations are performed at 37° C., and the filters are processed following hybridization for autoradiography. Plasmid DNA isolated from colonies identified as hybridizing with the 32P-labeled probes is subjected to automatic sequencing using the forward and reverse primers, as well as gene specific primer corresponding to nucleotides of the normal gene.

### EXAMPLE 2

Cells Transfected with Nucleic Acids Encoding SAgs

Cultured VX-2 carcinoma cells were shown to retain their tumorigenic activity after implantation into New Zealand white rabbits. Progressive tumor outgrowth was observed over a 3 week period. Nucleic acid encoding SEB isolated and characterized by Gaskill et al, J. Biol. Chem. 263:6276 (1988) and Ranelli et al., Proc. Natl Acad. Sci. USA 82:5850 (1985) were used to transfect tissue cultured VX-2 carcinoma cells using transfection methodology described in Example 1. Transfectants were selected using G418 and the survival of SEB-transfected VX-2 carcinoma cells was observed. In additional experiments, attempts were made to transfect murine 205 and 207 tumor cells with nucleic acid encoding SEB(the kind gift from Dr. Saleem Khan) and Streptococcal pyrogenic exotoxin A (the kind gift of Dr. Joseph Ferretti). Successful transfection of murine MCA 205 and B16 cells by nucleic acids encoding SEA and SEC2 was achieved shortly thereafter by integrating the SAg DNA into several retroviral vectors (MFG NEO) containing a growth hormone leader sequence under the control of a chick B-actin promoter (Krause J C et al., J. Hematotherapy 6: 41-51 (1997)). In addition, murine tumors MCA 205 fibrosarcoma cells and a spontaneous mammary carcinoma cells were successfully transfected with nucleic acids encoding SEB (provided by Dr. Saleem Khan) using the b-actin promoter. Transfected mammary carcinoma cells induced T cell proliferation in vitro. To demonstrate the anti-tumor capacity of tumor cells transfected with nucleic acid encoding a SAg, these transfectants were injected i.p. into syngeneic hosts with established mammary carcinomas. These transfectants demonstrated a capacity to reduce micrometastases of wild type mammary tumor in vivo assessed in a clonogenic lung metastases assay. The anti-tumor effect produced by the SEB transfectants was enhanced significantly by the co-administration of tumor cells transfected with nucleic acids encoding the costimulating molecule B7-1.

### EXAMPLE 3

Naked SAg DNA and Cells Co-Transfected with SAg DNA and with Additional Nucleic Acid Encoding Anti-Tumor Motifs or Products

Nucleic acids encoding a SAg are injected in naked or plasmid form into a host with cancer as a means of activating T cells and initiating an anti-tumor response. They may also be used as a vaccine to prevent the occurrence or recurrence of tumor in a host. Under circumstances where it is desirable to activate CD4 cells to produce a TH-1 cytokine response the nucleic acid construct used to transfect cells contains immunostimulatory sequences such as unmethylated CpG sequences. Nucleic acids encoding SAgs may be co transfected into tumor cells together with nucleic acid encoding other constituents capable of promoting an anti-tumor response. A list of possible components of nucleic acid constructs for direct administration and/or transfection of tumor cells which are administered to the host is presented in Table II.

The nucleic acid construct or constructs are administered to a host intramuscularly, intradermally, systemically, parenterally, intratumorally, orally or locally (in the vicinity of the tumor). Alternatively, the construct is administered via a catheter or other devices known in the art into the tumor vasculature supplying all or part of a tumor. When the construct is injected systemically, the nucleic acid construct is directed to the tumor using an anti-tumor antibody or ligand specific for a tumor receptor or receptor on the tumor neovasculature or stroma. The antibody or ligand or other targeting structures are conjugated to the SAg nucleic acid construct in order to facilitate the introduction of the construct into tumor cells. Nucleic acid/polypeptide complexes or nucleic acid/viral complexes are used to target a specific receptor on the tumor vasculature or stroma.

Table II—Nucleic Acid Constructs and Cells

SAg-encoding DNA is used alone or together with DNA encoding other cell surface moieties useful in generating antitumor immunity. Genes or their products are shown in column 1, source information is shown in column 3, preferred cells to be transformed, transfected or transduced with the DNA are shown in column 2. All of references are incorporated by reference in their entirety.

| Gene or Gene Product | Cells transformed | Reference or Source |
|---|---|---|
| 1. SAg (SEQ ID NOS: 67-68) | Tumor | [See text] |
| 2. Enterotoxin (SEQ ID NOS: 7-16) | Tumor | [See text] |
| 3. SAg receptor (SEQ ID NOS: 67-68) | Tumor | [See text] |
| 4. Enterotoxin receptor | Tumor | [See text] |
| 5. CD1 receptor(s) (SEQ ID NOS: 69-70) | Tumor | Martin, LH et al, Proc. |
| | | Natl. Acad. Sci. |
| | | 83: 9154-9158 (1986) |
| 6. CD14 receptor (SEQ ID NOS: 71-72) | Tumor | Ferrero, E et al., J. Immunol. |
| | | 145: 331-336 (1990) |
| 7. CD44 encoding nucleic acids (SEQ ID NO: 73) | T or NKT | Nottenburg, C et al. Proc. |
| | | Natl. Acad. Sci. 66: 8521-8525 |
| | | (1992) |
| 8. Carbohydrate modifying enzymes (SEQ ID NO: 74) | Tumor, T or NKT | Sheng, Y et al. Int. J. |
| | | Cancer 73: 850-858 (1997) |
| 9. TCR Vβ chain (SEQ ID NOS: 75-76) | Tumor | Tillinghast, JP et al, Science 233: |
| | | 879-883 (1986) |
| 10. Staph/Strep hyaluronidase(SEQ ID NOS: 77-78) | Tumor | Hynes WL et al., Infect. Immun., |
| | | 63: 3015-3020 (1995) |
| 11. Staph/Strep erythrogenic toxin(SEQ ID NOS: 79-80) | Tumor | McShan WM, et al., Adv. Exp. |
| | | Med. Biol. 418: 971-973 (1997) |
| 12. Staphylococcal β-hemolysin(SEQ ID NOS: 81-82) | Tumor | Projan SJ et al., Nucleic Acid Res. |
| | | 3305-3309 (1989) |
| 13. Strep capsular polysaccharide(SEQ ID NOS: 83-84) | Tumor | Lin, WS et al., J.Bacteriol. 176: |
| | | 7005-7016 (1994) |
| 14. Staph staphylocoagulase(SEQ ID NOS: 85-86) | Tumor | Kaida S. et al., J.Biochemistry |
| | | 102: 1177-1186 (1987) |
| 15. Staph Protein A (SEQ ID NOS: 87-88) | Tumor | Shuttleworth, HL et al., Gene 58: |
| | | 283-295 (1987) |
| 16. Staph Protein A domain D(SEQ ID NOS: 89-90) | Tumor | Roben, PW et al., J. |
| | | Immunol. 154: 6347-6445 |
| | | (1995) |
| 17. Staph Protein A Domain B(SEQ ID NO: 91) | Tumor | Gouda, H et al., Biochemistry, 31: |
| | | 9665-9672 (1992) |
| 18. Immunostimulatory protein | Tumor, T or NKT | Tokunaga, T et al., |
| | | Microbiol. Immunol. 36: 55-66, |
| | | (1992) |
| 19. Costimulatory protein | Tumor | Entage, PC et al., J.Immunol. |
| | | 60: 2531-2538 (1998) |
| 20. SAg-mimicking nucleic acid | T or NKT | |
| 21. Glycophorin (SEQ ID NOS: 92-93) | Tumor | Siebert, PD. et al., Proc. Natl. |
| | | Acad. Sci. USA 83 |
| | | 1665-1669 (1986) |
| 22. Mannose receptor (SEQ ID: NOS: 94-95) | Tumor | Kim SJ. et al., Genomics 14: |
| | | 721-727 (1992) |
| 23. Angiostatin (SEQ ID: NO: 96) | Tumor | Cao, Y. et al., J.Clin. Invest |
| | | 101: 1055-1063 (1998) |
| 24. Chemoattractant (SEQ ID NOS: 97-98) | Tumor | Ames, RS. et al., J. Biol. |
| | | Chem. 271: 20231-20234 |
| | | (1996) |
| 25. Chemokine (SEQ ID NOS: 99-100) | Tumor | Nagira, M et al., J. Biol. |
| | | Chem. 272: 19518-19524 |
| | | (1997) |
| 26. Transcription factor (SEQ ID NO: 101 | Tumor, T or NKT | Schwab M et al, Mol. Cell |
| | | Biol. 6: 2752-2758 (1986) |
| 27. Transcription factor-binding | Tumor, T or NKT | |
| nucleic acid | | |
| 28. SAg/peptide conjugate | Tumor | |
| 29. Glyco-SAg | Tumor | |
| 30. Staph. global regulator gene agr (SEQ ID NOS: 102-104) | Tumor | Balaban, N. et al, Proc. Natl. |
| | | Acad. Sci. USA 92: |
| | | 1619-1623 (1995) |
| 31. Lipid A biosynthetic(SEQ ID NOS: 105-112) | Tumor | Schnaitman CA et al., |
| genes lpxA-D | | Microbiological Reviews 57: |
| | | 655-682 (1993) |
| 32. Mycobacterial mycolic acid(SEQ ID NOS: 113-114) | Tumor | Fernandes ND et al., Gene |
| biosynthetic genes | | 170: 95-99 (1996); Mathur M |
| | | et al., J.Biol. Chem. |
| | | 267:19388-19395 (1992) |
| 33. c-abl oncogene amplified in(SEQ ID NOS: 115-116) | Tumor | Scherle PA et al., |
| chronic myel. Leukemia | | Proc. Natl. Acad. Sci. USA |
| | | 87: 1908 (1990); |
| | | Heisterkamp N et. al., Nature |
| | | 344: 251-253 (1990) |
| 34. erbB2 (HER2/neu) oncogene(SEQ ID NOS: 117-118) | Tumor | Schechter AL et al., Science |
| | | 229: 976 (1985); Bargmann |
| | | CL Nature 319: 226 (1986); |
| | | Hung MC et al., Proc. Natl. |
| | | Acad Sci. 83: 261 (1986); |
| | | Yamamoto T et al., Nature |
| | | 319: 230 (1986) |
| 35. IGF-1 receptor gene(SEQ ID NOS: 1189-120) | Tumor | Abbott AM et al., J. Biol. |
| | | Chem. 267: 10759-10763 |
| | | (1992); Scott J et al., Nature |
| | | 317: 260-262 (1985); Liu J et |
| | | al., Cell 75: 59-63 (1993) |
| 36. VEGF (SEQ ID NOS: 121-122) | Tumor | Tischer E et al., J. Biol. |
| | | Chem. 266: 11947-11954 |
| | | (1991) |
| 37. Strep emm-like gene family | Tumor | Kehoe MA, In: Cell-Wall |
| | | Associated Proteins in |
| | | Gram-Positive Bacteria in |
| | | Bacterial Cell Wall, Ghuysen |
| | | JM et al., eds, Elsevier, |
| | | Amsterdam, 1994 |
| 38. iNOS (SEQ ID NOS: 123-124) | Tumor | Xie QW et al., Science 256: |
| | | 225-228 (1992) |
| 39. Apolipoproteins (e.g., Lp(a), | Tumor | [See Text] |
| apoB-100, apoB-48, apoE) (SEQ ID NOS: 125-130) | | |
| 40. LDL & oxyLDL receptors | Tumor | [See Text] |
| (e.g., LDL oxyLDL, acetyl-LDL, | | |
| VLDL, LRP, CD36, SREC, LOX-1, | | |
| macrophage scavenger receptors) (SEQ ID NOS: 131-142) | | |

Chemical Conjugation of SAg Nucleic Acids to VTs, Apolipoproteins, HPV Epitopes or Other Polypeptides/Proteins Listed in Tables I and II.

The following section describes actual physical conjugates between poly- or oligonucleotides and peptides or proteins. SAg nucleic acid conjugates are prepared by chemical modification of nucleic acids at specific sites within individual nucleotides or within oligonucleotides such that a protein can be bound to a DNA or RNA polymer.

Derivatization may be accomplished through discrete sites on the available bases, sugars, or phosphate groups to create primary amines, sulfhydryls, carboxylates or phenolates. The chemical modification of nucleic acids can encompass several strategies. The initial derivatization may be the addition of a spacer arm to a particular reactive group on the nucleotide structure. Such a spacer typically contains a terminal functional group, such as an amine, that can be used to couple another molecule. The spacer may be used to react with a cross-linking agent, such as a heterobifunctional compound that can facilitate the conjugation of a protein or another molecule to the modified nucleotide.

If enzymatic methods are used to incorporate a small spacer into an oligonucleotide, subsequent chemical conjugation steps still are needed to add the protein moiety. In some cases, if an oligonucleotide contains the appropriate functional group, a protein may be directly coupled using chemical methods. Many of the chemical derivatization methods employed in these strategies involve the use of an activation step that produces a reactive intermediary. The activated species then can be used to couple a molecule containing a nucleophile, typically a primary amine.

A preferred method is to amidate the 5′ PO4 of the oligonucleotide with EDC and then couple cystamine to the 5′ amidated oligonucleotide. EDC will add an amide to the oligonucleotide to form a phosphoramidate linkage. After the addition of cystamine the disulfide is reduced with an agent such as dithiothreitol (DTT) to produce a free 5′ sulfhydryl. The derivatized oligonucleotide is then coupled to a protein chain (e.g., a verotoxin A or B chain) that has been activated with a heterobifunctional cross-linker such as succinimidyl 4(N-maleimidomethyl)cyclohexane 1-carboxylate (SMCC) which reacts with the amines on the protein which then react with the sulfhydryls on the derivatized oligonucleotide. N-succinimidyl S-actylthioacetate (SATA) is useful for adding a free thiol or sulfhydryl group to a molecule lacking this moiety. With this modification, “protected” sulfhydryl is formed which may be stored indefinitely in this protected state.

When needed, the acetyl group on the protected sulfhydryl is removed to reveal the sulfhydryl for conjugation to another molecule. A heterobifunctional agent such as SMCC or N-Succinimidyl 3-(2-pyridylthio)propionate (SPDP) may be directly added to the amidated oligonucleotide phosphate group to produce a free sulfhydryl unit for reactivity with the protein or peptide.

Chemical Conjugation of Polypeptides/Proteins to SAg DNA Via Carbodiimide Reaction with the 5′-Phosphates (Phosphoramidate Formation)

The water-soluble carbodiimide EDC, rapidly reacts with a carboxylate or phosphate to form an active complex able to couple with a primary amine-containing compound. The carbodiimide activates an alkyl phosphate group to a highly reactive phosphodiester intermediate. Diamine spacer molecules or amine-containing peptides then may react with this active species to form a stable phosphoramidate bond. Alternatively, bis-hydrazide compounds may be coupled to DNA using this protocol to yield a terminal hydrazide functional group able to react with aldehyde-containing molecules (Ghosh et. al., 1989). These methods permit specific labeling of SAg DNA only at the 5′ end. The following protocol describes the modification of SAg DNA or RNA oligonucleotides at their 5′-phosphate ends with a bis-hydrazide compound, such as adipic acid dihydrazide or carbohydrazide. A similar procedure for coupling the diamine compound cystamine is described below.

Protocol

1. Weigh out 1.25 mg of the carbodiimide 1-ethyl-3-(3-dimethylamino-propyl)carbodiimide hydrochloride (EDC) into a microfuge tube.

2. Add 7.5 ml of SAg RNA or DNA that has 5′ phosphate groups. The concentration of the oligonucleotide should be 7.5-15 nmol or a total of about 57-115.5 mg. Also immediately add 5 ml of 0.25 M bis-hydrazide compound dissolved in 0.1 M imidazole, pH 6.

3. Mix (e.g., by vortexing) and centrifuge in a microfuge for 5 min at maximal rpm.

4. Add an additional 20 ml of 0.1 M imidazole, pH 6. Mix and allow to react for 30 mm at room temperature.

5. Purify the hydrazide-labeled oligonucleotide by gel filtration on Sephadex G-25 using 10 mM sodium phosphate, 0.15 M NaCl, 10 mM EDTA, pH 7.2. The oligonucleotide now may be conjugated with an aldehyde-containing molecule.

Sulfhydryl Modification of SAg DNA

Creating a sulfhydryl group on SAg DNA allows conjugation reactions to be done with sulfhydryl-reactive heterobifunctional cross-linkers providing increased control over the derivatization process. Proteins are activated with a cross-lining agent containing an amine-reactive and a sulfhydryl-reactive end, such as SPDP, leaving the sulfhydryl-reactive portion free to couple with the modified DNA molecule. Having a sulfhydryl group on the SAg DNA directs the coupling reaction to discrete sites on the nucleotide strand, thus better preserving hybridization ability in the final conjugate. In addition, heterobifunctional cross-linkers of this type allow two- or three-step conjugation procedures which result in better yield of the desired conjugate than do homobifunctional reagents.

Cystamine Modification of 5′ Phosphate Groups on Superantigen Nucleotides Using EDC

SAg DNA or RNA is modified with cystamine at the 5′ phosphate groups using the carbodiimide reaction described above. In some procedures, the reaction is carried out in a two-step process by first forming a reactive phosphorylnimdazolide by EDC conjugation in an imidazole buffer. Next, cystamine is reacted with the activated oligonucleotide, causing the imidazole to be replaced by the amine and creating a phosphoramidate linkage. Reduction of the cystamine-labeled oligonucleotide using a disulfide reducing agent releases 2-mercaptoethylamine and creates a thiol group.

Protocol

1. Weigh out 1.25 mg of the carbodiimide 1-ethyl-3-(3-dimethylamino-propyl)carbodiimide hydrochloride (EDC) into a microfuge tube.

2. Add 7.5 ml of SAg RNA or DNA that has 5′ phosphate groups. The concentration of the oligonucleotide should be 7.5-15 nmol or a total of about 57-115.5 mg. Also immediately add 5 ml of 0.25 M cystamine in 0.1 M imidazole, pH 6.

3. Mix (e.g., by vortexing) and centrifuge in a microfuge for 5 min at maximal rpm.

4. Add an additional 20 ml of 0.1 M imidazole, pH 6. Mix and allow to react for 30 mm at room temperature.

5. For reduction of the cystamine disulfides, add 20 ml of 1 M DTT and incubate at room temperature for 15 mm. This will release 2-mercaptoethylamine from the cystamine modification site and create the free sulfhydryl on the 5′ terminus of the oligonucleotide.

6. Purify the SH-labeled oligo by gel filtration on Sephadex G-25 using 10 mM sodium phosphate, 0.15 M NaCl, 10 mM EDTA, pH 7.2. The oligonucleotide now may be used to conjugate with an activated protein containing a sulfhydryl-reactive group.

SPDP Modification of Amines on Superantigen Nucleotides

SAg DNA that has been modified with an amine-terminal spacer arm may be thiolated to contain a sulfhydryl residue. Theoretically, any amine-reactive thiolation reagent may be used to convert an amino group on a SAg DNA molecule into a thiol. A preferred reagent both for cross-linking and for thiolation reactions is the heterobifunctional reagent SPDP. The NHS ester end of SPDP reacts with primary amine groups to produce stable amide bonds. The other end of the cross-linker contains a thiol-reactive pyridyldisulfide group that also can be reduced with DTT to create a free sulfhydryl. The reaction of a 5′-diamine-modified SAg DNA oligonucleotide with SPDP proceeds under mildly alkaline conditions (optimal pH 7-9) yields the pyridyldisulfide-activated intermediate. This derivative can be used to couple directly with sulfhydryl-containing compounds, or it may be converted into a free sulfhydryl for coupling to thiol-reactive compounds. In an alternative approach, 2,2′-dipyridyldisulfide is used to create reactive pyridyldisulfide groups on a reduced 5′-cystamine-labeled SAg oligonucleotide. This derivative then can be used to couple with sulfhydryl-containing molecules, forming a disulfide bond. Reduction of the pyridyldisulfide end after SPDP modification releases the pyridine-2-thione leaving group and generates a terminal-SH group.

Protocol

1. Dissolve the amine-modified SAg oligonucleotide to be thiolated in 250 ml of 50 mM sodium phosphate, pH 7.5.

2. Dissolve SPDP at a concentration of 6.2 mg/ml in DMSO to make a 20 mM stock solution. Alternatively, LC-SPDP may be used and dissolved at a concentration of 8.5 mg/ml in DMSO (also makes a 20 mM solution). If the water-soluble Sulfo-LC-SPDP is used, a stock solution in water may be prepared just prior to addition of an aliquot to the thiolation reaction. In this case, prepare a 10 mM solution of Sulfo-LC-SPDP by dissolving 5.2 mg/ml in water. Since an aqueous solution of the cross-linker will degrade by hydrolysis of the sulfo-NHS ester, it should be used quickly.

3. Add 50 ml of the SPDP (or LC-SPDP) solution to the SAg oligonucleotide solution. Add 100 ml of the Sulfo-LC-SPDP solution, if the water-soluble cross-linker is used. Mix.

4. Allow to react for 1 h at room temperature.

5. Remove excess reagents from the modified SAg oligonucleotide by gel filtration. The modified oligonucleotide now may be used to conjugate with a sulfhydryl-containing molecule, or it may be reduced to create a thiol for conjugation with sulfhydryl-reactive molecules.

6. To release the pyridine-2-thione leaving group and form the free sulfhydryl, add 20 ml of 1M DTT and incubate at room temperature for 15 mm. If present in sufficient quantity, the release of pyridine-2-thione is followed by its characteristic absorbance at 343 nm (e=8.08×10³ M⁻¹ cm⁻¹). For many oligonucleotide modification applications, however, the leaving group will be present in too low a concentration to be detectable.

7. Purify the thiolated oligonucleotide from excess DTT by dialysis or gel filtration using 50 mM sodium phosphate, 1 mM EDTA, pH 7.2. The modified oligonucleotide should be used immediately in a conjugation reaction to prevent sulfhydryl oxidation and formation of disulfide cross-links.

N-succinimidyl S-actylthioacetate (SATA ) Modification of Amines on Superantigen DNA Nucleotides

SAg oligonucleotides containing amine groups introduced by enzymatic or chemical means may be modified with SATA to produce protected sulfhydryl derivatives. The NHS (N-hydroxysuccinimide) ester end of SATA reacts with a primary amine to form a stable amide bond. After modification, the acetyl protecting group can be removed as needed by treatment with hydroxylamine under mildly alkaline conditions. The result is terminal sulfhydryl groups that can be used for subsequent labeling with thiol-reactive probes or activated-protein derivatives.

Protocol

1. Dissolve the amine-modified SAg oligonucleotide to be thiolated in 250 ml of 50 mM sodium phosphate, pH 8.

2. Dissolve SATA in DMF at a concentration of 8 mg/ml.

3. Add 250 ml of the SATA solution to the oligo solution. Mix.

4. React for 3 h at 37° C.

5. Remove excess reagents by gel filtration.

6. To deprotect the thioacetyl group, add 100 ml of 50 mM hydroxylamine hydrochloride, 2.5 mM EDTA, pH 7.5, and react for 2 h.

7. The sulfhydryl-containing oligonucleotide may be used immediately to conjugate with a sulfhydryl-reactive label, or it can be purified from excess hydroxylamine by gel filtration.

Conjugation of a Polypeptide to SAg DNA

As indicated, the DNA molecule must be modified to contain one or more suitable reactive groups, such as nucleophiles like amines or sulfhydryls. The modifications that employ enzymatic or chemical methods can result in random incorporation of modification sites or can be directed exclusively to one end of the DNA molecule, e.g., 5′ phosphate coupling.

Some of the more common procedures for preparing DNA-polypeptide conjugates are given below.

Polyieptide (e.g., VT) Conjugation to Cystamine-Modified SAg DNA Using Amine- and Sulfhydryl-Reactive Heterobifunctional Cross-Linkers

Cystamine groups are added to the 5′ phosphate of SAg DNA as described above. Once a sulfhydryl-modified DNA has been prepared, the following protocol may be used. The protein is activated with SPDP. Reacting the SAgic DNA probe in excess allows easy separation of uncoupled SAg oligonucleotide from conjugated molecules.

Protocol

1. Dissolve a 5′-sulfhydryl-modified SAg oligonucleotide in water or 10 mM EDTA at a concentration of 0.05-25 mg/ml. Calculate the total nanomoles of oligonucleotide present based on its molecular weight.

2. Add 0.15M NaCl, 10 mM EDTA, pH 7.2. Add the oligonucleotide solution to the activated protein in a 10-fold molar excess.

3. React at room temperature for 30 mm with gentle mixing.

4. The protein-DNA conjugate is purified away from excess SAg oligonucleotide by dialysis or gel filtration, or through the use of centrifugal concentrators. Centricon-30 concentrators (Amicon) that have a molecular weight cutoff of 30,000 are also used to remove unreacted oligonucleotides. Since the polypeptide molecular weight is approximately 140,000 and the conjugate is even higher, a relatively small DNA oligomer will pass through the membranes of these units while the conjugate will not. To purify the prepared conjugate using Centricon-30s, add 2 ml of the phosphate buffer from step 2 to one concentrator unit, then add the reaction mixture to the buffer and mix. Centrifuge at 1000 g for 15 mm or until the retentate volume is about 50 ml. Add another 2 ml of buffer and centrifuge again until the retentate is 50 ml. Invert the Centricon-30 unit and centrifuge to collect the retentate in the collection tube provided by the manufacturer.

Administration of Peptide—DNA (pDNA), Naked DNA, or Protein or Peptide Conjugates

Naked DNA, pDNA, nucleic acid-peptide or -polypeptide conjugates or genetic fusion products are administered parenterally (for example, iv, ip, im, subcutaneously, intrathecally, intratumoral, rectally, transcutaneously) or orally. Administration may also be by a gene gun using a 1 ml syringe and a 28 gauge needle. The nucleic acid is administered intradermally or intramuscularly in a total volume of 100 ml. A Tyne applicator is used to deliver doses of 1-1000 mg of DNA at 3× weekly intervals. SAg-encoding nucleic acid is injected directly into the tumor. The nucleic acid either contains or does not contain immunostimulatory sequences that induce activation of T cells and skew the response toward production of TH1 cytokines. For example, if nucleic acids encoding a tumor associated antigen are used then the nucleic acids are engineered to incorporate ISS sequences in order to fully activate a TH1 response. Likewise, if nucleic acid encoding a tumor associated antigen is cotransfected with nucleic acid encoding a SAg, then one of the nucleic acid constructs is engineered to contain an ISS.

Viral DNA, nucleic acid expression cassettes or plasmids or bacteriophages encoding the constructs given in Table II may be used for in vivo immunization in place of naked DNA. Viruses may also acquire the α-Gal epitope after transfection into tumor cells which contain the a-galactosyltransferase enzyme either naturally or via transfection. The virus must possess the intact N-acetyllactosamine substrate for the galactosyl-transferase in order to express the α-Gal. The viruses shedding from these cells will express the aGal epitope. The virus also contains peptide sequences for SAg and tumor associated antigen acquired from the tumor cells which were previously transfected with nucleic acids encoding SAg and tumor antigen. The shed virus may also express staphylococcal or streptococcal hyaluronidase and capsular polysaccharide sequences obtained from host tumor cell or accessory cells previously transfected with nucleic acids encoding these genes. The shed virus expressing α-Gal, SAg, hyaluronidase and capsular polysaccharide is capable of initiating a potent tumoricidal response when administered to hosts with established tumors or when used as a tumor vaccine against potential tumors.

These constructs are also used as vaccines. Further, the nucleic acid construct is pre-processed ex vivo in muscle cells before selective delivery into host tumor tissue. Cationic liposomes or other liposomes or drug carriers well known in the art are used as vehicles to deliver the nucleic acids in vivo.

The transfection process is also carried out ex vivo. Nucleic acids encoding SAgs together with the nucleic acid constructs given in Table II are transfected into tumor cells of all types and antigen presenting cells such as MHC class I and class II as well as APCs expressing CD1 and mannose receptors. These include but are not limited to DCs, immunocytes, monocytes, macrophages, and fibroblasts. SAg is transfected alone or together with one or more of the above constructs given in Table II. The transfected cell expresses/secretes preferentially a SAg plus an immunogenic oncogene product, anti-angiogenesis factor, glycosylceramide, LPS or α-Gal. The transfectants present their gene products on cell surface receptors such as conventional MHC molecules for SAgs or in the case of the glycosylceramides or LPS on a CD-1 or mannose receptor. (APC). Glycosylated SAgs show preference for presentation on mannose receptors

### EXAMPLE 4

SAgs, Tumor Antigens, Glycosylceramides, LPS's, Binary and Ternary Complexes Applied to MHC Class I, Class II, CD1 or Mannose Receptors

The above molecules and all of the conjugates given in section 55 are applied to antigen presenting receptors as given below. CD1 represents a family of non-polymorphic antigen presenting molecules unlinked to the MHC molecules expressed by most professional APCs. The NKT cells that recognize CD1 presented antigens express NKR-P1, Ly49 receptors, an invariant chain and a Vβ8.2 variable region. With respect to these receptors, they share identity and their natural ligands with NK cells. Specifically, CD1 binds peptides with extended NH2 and COOH termini flanking the core binding motif. Long peptides (greater than 8 to 10 amino acids) with amino acid residues at their hydrophobic binding sites and greatly restricted anchors are preferred. This recognition of CD1-presented antigens depends on the type and distribution of sugar residues. Mycobacterial cell wall antigens namely mycolic acids and lipoarabinomannan also bind to CD1. Recently several glycosylceramides, in particular, monogalactosyl ceramides GalCer) were shown to bind to CD1 and to activate NKT cells Specifically, CD1 molecules are capable of presenting mannosides with 1,2 linkages and a phosphatidylinositol unit. CD1 bound antigens are recognized by NKT cells α/βTCR TCR positive; CD4 and CD8 negative). For instance, NKT cells are activated by a lipoarabinomannan (LAM) presented on CD1 receptors and become cytolytic while producing abundant INF.

In the present invention, a SAg bound to a monogalactosylceramide such as GalCer is loaded onto CD1 or MHC class I or II receptors expressed by APCs. The CD1 or MHC receptors are in soluble or immobilized form produced by methods well described in the art. According to this invention, CD1 receptors present SAg polypeptides complexed with GalCer lipids or oligosaccharides to T cell and/or NKT cell population which recognize the conjugates and commence differentiation to tumor specific effector cells. These ligands are be loaded on the CD1 receptor sequentially, simultaneously or as a preformed conjugates. Alternatively, they are positioned on the CD1 receptor after internal processing of their nucleic acid counterparts in the antigen presenting cells. These cells are then harvested and used for adoptive immunotherapy (Examples 7, 15, 16. 18-23).

These complexes are also useful in vivo as a preventative or therapeutic antitumor vaccine (Example 14, 15, 16, 18-23).

SAgs and tumor associated antigen (TAA) are loaded sequentially on to class II receptors of antigen presenting cells. Alternatively, preformed complexes of tumor associated antigen and SAg are loaded onto MHC class II receptors. The SAg may be in the native or glycosylated form. The tumor associated antigen is also fused genetically to the b chain of the MHC class II receptor. A SAg is added once the TAA is expressed bound to the MHC class II. The sequence may also be reversed so that a SAg is genetically processed and bound to the b chain after which the TAA is added. Consensus or repeating nucleic acid sequences shared by a tumor associated antigen and a SAg are cloned into a single sequence and transfected into APCs which display the consensus peptide in the context of the class II receptor. Methodology for production of the fusion genes is well described in the art. (See Ausubel. F M et al., supra; Sambrook, J et al., supra) T cells or NKT cells are activated after exposure to SAg and TAA producing an expanded tumor specific T cell effector population which is useful in adoptive immunotherapy of cancer (Examples 7, 15. 16, 18-23).

Antigen presenting cells in this system are chosen from a group consisting of DCs, fibroblasts, macrophages, and lymphocytes, but other professional APCs or any other cell transfectants, phage displays or liposomes expressing the class I or class II receptors are also used. Alternatively, a tumor associated antigen is bound to an APC that is pharmacologically or genetically inhibited from antigen processing. SAg is added and the complex of SAg and protein bound to class II is then presented to a T cell population to produce a tumor specific effector cell population for use in adoptive immunotherapy of cancer as in Example 15, 16, 18-23). These complexes are also useful in vivo as a preventative or therapeutic antitumor vaccine (Example 14, 15, 16, 18-23).

Soluble SAg MHC class II proteins with covalently bound single peptides are produced using a baculovirus system to express in insect cells two murine class II molecules with peptides attached by a linker to the N terminus of their b-chains (Kozono H. et al., Nature 369: 151-154 (1994)). The resulting peptide is engaged by the peptide binding groove of the secreted MHC molecule and this complex is recognized by T cells bearing receptors specific for the combination. In this method, the approximately 100 bp fragment encoding the SAg and a flexible linker with an embedded thrombin cleavage site is introduced in frame by the PCR just after the third codon of the b1 domain. This assures a recognizable leader peptide cleavage site and flexible link between the C-terminus of the foreign peptide bound in the cleft of the MHC molecule and the N terminus of the b1 domain of SAg amino acids. Soluble complexes consisting of receptors and various SAg are prepared in this way and are used to activate T cells for use in adoptive immunotherapy. Similarly, preparations consisting of MHC class I receptors, CD1 or mannose receptors complexed with SAgs, glycosylceramides or LPS's are produced which are useful in activating T cells or NKT cells for adoptive immunotherapy of cancer in protocols given in Examples 7, 15, 16, 18-23). These complexes are also useful in vivo as a preventative or therapeutic antitumor vaccine (Example 14, 15, 16, 18-23).

To produce complexes composed of SAgs with class I or II MHC or soluble DR a or b (lacking the transmembrane domain) and TCR heterodimer, a soluble human TCR heterodimer which has specificity for various tumor associated antigens bound to the human class I or II MHC molecules or human soluble CD1 molecules is used. A typical system for preparing ternary SAg-tumor peptide-MHC or ternaryCD1-glycosylceramide (preferably GalCer)-SAg complexes capable of triggering T cells or NKT cells is as follows. CD1, DR-1 or HLA-A2 restricted tumor antigen specific T cell or NKT cell clones are used although primary unsensitized T or NKT cells may be used as well. The DR-1 and HLA-A2 homozygous Epstein-Barr virus-transformed B cell line LG-2 or DCs expressing CD1 receptors are used as APCs either live or fixed in 0.5% paraformaldehyde for 20 minutes. LG-2 and DCs (2.67×10⁵ per ml) in RPMI 1640 with 1% fetal bovine serum are pulsed with tumor antigen and glycosylceramide respectively for 2 hours at 37° C. and then washed in RPMI 1640/1% fetal bovine serum to remove unbound antigen. SAg is added for 2 hours at 37° C. Pulsed APCs (4×10⁴ per well) are co cultured with resting T cells or NKT cells (2×10⁴ per well) in round-bottom microtiter plates in RPMI 1640/5% human serum. Twenty four hours later, the cells are harvested. The APCs are separated and the T cells or NKT cells may be optionally expanded further with IL.-2 Optionally, complexes comprising soluble recombinant DRa or b chain with bound superantigen are presented to the T cell or NKT cells which are then expanded with IL-2. These cells are then harvested and used for adoptive immunotherapy (Examples 7, 15, 16. 18-23). The APC containing the complexes are also useful in vivo as a preventative or therapeutic antitumor vaccine (Example 14, 15, 16, 18-23). Also useful for tumor therapy are the complexes LIP⁺ GPI-SAg (from Section 38), either free or in the form of vesicles or exosomes comprising SAg-GalCer complexes or SAg-tumor peptide (including but not limited to normal mutated structures). The ternary complexes of SAg-GalCer-heat shock protein and tumor peptide-heat shock protein are also useful, These complexes may be in or soluble or immobilized form, attached to a CD1 or MHC or as part of a vesicle or exosome. the complexes are also useful in vivo as a preventative or therapeutic antitumor vaccine (Example 14, 15, 16, 18-23).

The tumor associated antigen or SAg-tumor associated antigen complex is conjugated to oxidized mannan (polymannose) by methods described by Apostolopoulos, V et al., Proc. Natl. Acad. Sci. USA 92: 10128-10132 (1995) which is then loaded onto mannose receptors of antigen presenting cells for stimulation of a T cell anti-tumor response. Alternatively, the SAg (optionally conjugated to tumor peptides)-mannan conjugate is administered to tumor bearing hosts by methods in Example 15, 16, 18-23).

The SAg alone or conjugated to a tumor associated antigen is recognized by the mannose receptor on macrophages. This requires a glycosylated SAg which is recognized by the mannose receptor on macrophages. A native or glycosylated tumor associated antigen-SAg conjugate or a consensus peptide of both polypeptides is presented to mannose receptors expressed on antigen presenting cells which are exposed to a T cell or NKT cell population to produce a tumor specific effector cells by methods in Example 15, 16, 18-23). These complexes are also useful in vivo as a preventative or therapeutic antitumor vaccine (Example 14, 15, 16, 18-23). They are also used ex vivo to produce a population of tumor specific effector T or NKT cells for the adoptive immunotherapy cancer by methods and protocols given in Examples 7, 15, 16, 18-23 and 36.

The mannose receptor delivers the complex to the late endosomal and lysosomal vesicles and the MHC class II loading compartment where the antigen is loaded onto CD1b molecules. The C1b molecule is endocytosed at the plasma membrane in coated pits and vesicle structures, transits to early endosomes and is then delivered to the MHC class II antigen loading compartment. The endosomal localization motif on the tail of the CD1b molecule is essential for antigen trafficking of CD1b through the lysosomal compartment required for loading of antigen into CD1b and its ultimate transport to the membrane. The antigen binding groove of CD1 is deeper and narrower than the MHC class I molecule groove containing a hydrophobic binding site which accommodates the lipid portion of the molecules such as lipoarabinomannan or GalCer and the SAg-LPS constructs given herein. APCs expressing the above constructs are exposed to NKT cell populations which recognize the antigens in the context of the CD1 receptor. If carried out ex vivo this results in the formation of tumor specific effector NKT cells which are used for adoptive immunotherapy by protocols given in Examples 7, 15, 16, 18-23).

### EXAMPLE 5

SAg Conjugation to Glycosylceramides, Gangliosides, LPS's, Glycans, Peptidoglycans Phytosphingolipids, Lipoproteins, oxyLDL and Lipoarabinomannans

All of the SAg-lipid conjugates given above and in section 55 are prepared as follows. Selection of the SAg peptide to be used for coupling is governed by several criteria. In practice, a 10-15 residue peptide is selected. For SAgs, the sites chosen for coupling are those presumed not to be vitally involved in T cell binding and activation. In most SAgs, these sites are broadly distributed throughout the molecule. They are available at flexible regions of the protein and on reverse turns or loop structures. C termini are more mobile than the rest of the molecule and frequently exposed on the protein surface. This region is accessible to be coupled to another ligand especially using m-maleimidobenzoyl-N-hydroxysuccinimide ester (MBS) via a Cys residue that has been added to the N terminus of the peptide. By coupling the peptide via its N-terminal end, the peptide is exposed in a fashion similar to that found in the native antigen. Additional criteria for selection of the coupling site such as exposed hydrophilic regions, secondary structure, hydropathicity profiles, and probability of helix formation may not be useful. However, care is take not to disrupt predicted polysaccharide attachment sites, most notably the sequence Asn-X-Ser or Asn-X-Thr, which predicts the presence of Asn-linked polysaccharide moieties. In addition to location of transmembrane regions, Asn-linked glycosylation sites and sites of signal sequence cleavage are all important. After due consideration, the C using 7-15 residues terminus is preferred and is modified to accommodate MBS. This procedure requires a free sulfhydryl group on the synthetic peptide and free amino groups on the ligand. Therefore, to use this method, it is necessary to add a Cys residue to the C or N terminus of the peptide.

Biochemical Conjugation Methods:

SAgs are conjugated to polysaccharide containing structures using several methods well described in the art (Hermanson, G T Bioconjugate Techniques Academic Press, San Diego, Calif. 1996). Two methods are given here one utilizing the isolated complex carbohydrate obtained from the purified ganglioside which is then chemically conjugated to SAg and in another method wherein the ganglioside and SAg are both incorporated into a liposomal membrane. Either method is used to produce complexes which are included within the scope of this invention. However they are by no means exhaustive of all the techniques which could be employed to conjugate human tumor antigens to SAg molecules. Other conjugation strategies may be utilized to produce an immunologically active complex as described by this invention. (See Offord, R E. in Protein Engineering ed. A R Rees, Oxford, 1992)

Direct Conjugation of Ganglioside, LPS or Peptidoglycan to SAg Molecules

1. Ganglioside or LPS antigens are purified and are then dissolved in aqueous solution at pH 6.0 at a concentration of 1.0 mM/ml

2. Endoglycoceramidase from Rhodococcus (Genzyme) is added to the ganglioside solution to a level of 5 milliunits. The solution is incubated overnight at 37° C. with gentle agitation. The endoglycoceramidase specifically cleaves at the ceramide-polysaccharide bond liberating ceramide and clipping off the complex carbohydrate making up the ganglioside

3. The polysaccharide is isolated by HPLC size exclusion chromatography or by ultrafiltration

4. SAg is dissolved in 1M sodium phosphate, 0.15 M NaCl, pH 7.5, at a concentration of 1 mg/ml. The purified polysaccharide antigen is added to this solution to a concentration of at least 1 mM/ml.

5. In a fume hood, 20 microliters of 5 M sodium cyanoborohydride solution in 1 M NaOH (Aldrich) is added to each ml of the SAg solution.

6. The reaction is mixed gently and incubated at room temperature for 72 hours or 4° C. for 1 week. This reaction reductively aminates the reducing end of the polysaccharide (at the point it was cleaved by the endoglycoceramidase) to the amine groups on the SAg protein creating stable conjugate coupled through a secondary amine linkage. The degree of polysaccharide coupling can be controlled by limiting the time of reaction.

7. Remove unreacted carbohydrate and cyanoborohydride by gel filtration on Sephadex G-25 or by dialysis.

In a second method, SAg-GalCer, SAg-GalCer-CD1, SAg-glycosphingolipid, or SAg-glycosphingolipid-CD1 complexes or SAg conjugates given in secton 55 are produced which have the added benefit of presenting the glycosylceramide in a polyvalent array which is important for high affinity binding to complementary receptors. They retain nearly all of their original structure including most of the ceramide moiety and the entire oligosaccharide chain. The principle of preparation derived from Mahoney, J A et al., Meth. Enzymol 242: 17-27 (1994) is as follows. The fatty acid amide is hydrolyzed from the intact ganglioside converting it to its lyso form which has a unique primary amine at the 2-position of sphingosine. The lysoganglioside is treated with a bifunctional cross-linking reagent, succinimidyl 4(N-maleimidomethyl)cyclohexane 1-carboxylate (SMCC), which forms an amide bond to the 2-position of sphingosine and results in a sulfhydryl-reactive maleimidyl moiety attached through a linker arm, to the original position of the fatty acid amide on the ceramide portion of the ganglioside. The SAg protein is treated with a reagent, N-succinimidyl S-acetylthioacetate (SATA), which converts the lysine e-amino groups to acetylated sulfhydryls. Subsequent treatment with hydroxylamine reveals the desired free sulfhydryls. Treatment of sulfhydryl-derivatized SAg with maleimidyl derivatized ganglioside results in a stable thioester linkage between the ganglioside and the protein. The final product is chromatographically purified and characterized by protein and carbohydrate analysis. The SAg-GalCer or SAg-glycosphingolipid complex is then loaded onto a soluble CD1 receptor.

LPS's and peptidoglycans are conjugated to SAg by methods well described in the art. The most convenient and preferred method to target specifically the polysaccharides on the protein is through mild sodium periodate oxidation. Periodate cleaves adjacent hydroxyl groups in sugar residues to create highly reactive aldehyde functional groups. The generated aldehydes are used to in coupling reactions with amine or hydrazide containing molecules to form covalent linkages. Amines react with formyl groups under reductive amination conditions using a suitable reducing agent such as sodium cyanoborohydride. The result of the reaction is a stable secondary amine linkage. Hydrazides spontaneously react with aldehydes to form hydrazone linkages, although the addition of a reducing agent greatly increases the efficiency of the reaction and the stability of the bond. (See Hermanson, G T. Bioconjugate Techniques, Academic Press, San Diego Calif. 1996).

Production of Liposomes Displaying Glycolipid or Apolipoprotein or oxyLDL-SAg Complexes

Liposomes composed of the highly immunogenic constructs described herein are prepared. They may include lipoproteins such as SAgs coupled to Gal, GalCer, SAg-glycosphingolipid, SAg-glycosylceramides, SAg-phytosphingolipids, SAg-mycosphingolipid and SAg-lipid conjugates given in section 55. Liposomes comprising SAgs conjugated to apolipoproteins or oxyLDL receptors are useful for targeting endothelial or macrophage oxyLDL receptors in tumor microvasculature. Other SAg conjugates e.g., SAg-glycosphingolipid, SAg-glycosylceramides, SAg-phytosphingolipids, SAg-mycosphingolipid are useful in immunizing T cells, NK cells and NKT cells. Cationic liposomes are also useful as a means of transferring the nucleic acid constructs of this invention to tumor tissue. GalCer (a monogalactosylceramide) comprises the major portion of the liposome. The most effective lengths of fatty acyl chain and sphingosine (or ceramide) base are C26 and C18 respectively and a phytosphingosine backbone. Sphingolipids lend structural advantages to the integrity of liposomal membranes and have prolonged duration in vivo. The Gal carbohydrate epitope is linked to liposomes via the amphipathic properties of the surface sphingolipids. The Gal is converted to a glycolipid with a sphingosine backbone possessing a hydrophobic fatty acid tail that embeds them into membrane bilayers. The hydrophilic carbohydrate ends of these amphipathic molecules can interact with molecules dissolved in the surrounding environment. Sphingosine glycolipids consisting of lactosylceramide, GalGal(1-3)Gal(1-4)GlcNAc—R) or glycosphingolipids with terminal Gal(α 1-4)Gal are prepared in a manner similar to that of sphingolipids. All methods of preparation of liposomes have several steps in common: (1) dissolution of the lipid mixture in an organic solvent, (2) dispersion in an aqueous phase, and (3) fractionation to isolate the correct liposomal population.

In the first stage, the desired mix of lipid components is dissolved in organic solvent (usually chloroform:methanol (2:1 by volume) to create a homogenous mixture. This mixture includes any phospholipid derivatized to contain reactive groups as well as other lipids used to form and stabilize the bulk of the liposomal structure. The correct ratio of lipid constituents to form stable liposomes is important A reliable liposomal composition for encapsulating aqueous substances contains molar ratios of lecithin:cholesterol:negatively charged phospholipid (e.g., phosphatidyl glycerol) of 0.9:1:0.1. Apolipoproteins (e.g., LP(a)) or oxyLDL (e.g., 7β-hydroperoxycholesterol or 7β-hydroperoxy-choles-5-en-3β-ol) can substitute for cholesterol in the preparation of the liposomes. In general, to maintain membrane stability, the PE derivative should not exceed a concentration ratio of about 1-10 mol PE per 100 mol of total lipid. Once the desired mixture of lipid components is dissolved and homogenized in organic solvent, several techniques are used to disperse the liposomes in aqueous solution. These methods are broadly classified as (1) mechanical dispersion, (2) detergent-assisted solubilization, and (3) solvent-mediated dispersion. With mechanical dispersion to form vesicles, the lipid solution is dried to remove all traces of organic solvent prior to dispersion in aqueous media. The dispersion process is key to producing liposomal membranes of the correct morphology. Methods utilized include simple shaking, high pressure emulsification, sonication, extrusion through small-pores membranes and various freeze-thaw techniques. Detergent-assisted solubilization is also used to bring the lipid more effectively into the aqueous phase for dispersion. Triton X, alkyl glycosides or bile salts such as sodium deoxycholate are employed. Other modalities or dispersion include the steps of dissolving phospholipids and other lipid to be part of the liposomal membrane in ethanol. This ethanolic solution is then rapidly injected into an aqueous solution of 0.16 M KCl using a Hamilton syringe resulting in a maximum concentration of no more than 7.5% ethanol. Using this method, single bilayer liposomes of about 25-nm diameter are produced. To remove the excess aqueous components that are not encapsulated during the vesicle formation, gel filtration using Sephadex G-50 or dialysis is employed. To fractionate the liposome population according to size, gel filtration is carried out using a column of Sepharose 2B or 4B

SAgs are conjugated to the GalCer or glycosphingolipids with terminal Gal(α 1-4)Gal, apolipoproteins, LDL or oxyLDL or LDL receptors before incorporation into the liposomal membrane or they may be incorporated into the membrane during the preparation of the liposomal membrane. Likewise, the SAg is conjugated to GalCer or glycosphingolipids with terminal Gal(α 1-4)Gal at the glycolipid's polar head region by methods well known in the art including using heterobifunctional crosslinkers or periodate oxidation techniques. Alternatively, after the GalCer or glycosphingolipids with terminal Gal(α 1-4)Gal is incorporated into the membrane, the liposomes are derivatized for further binding to the SAg proteins using the sodium periodate which oxidizes the ceramide's free hydroxyl to an aldehyde which is further modified by reductive amination. Using the phosphatidylethanolamine of the lipid in the liposome, SAgs are coupled to the liposome using various bifunctional agents including carbodiimide, glutaraldehyde, dimethyl suberimidate, periodate oxidation followed by reductive amination, N-succinimidyl 3-(2-pyridyldithio)propionate (SPDP), succinimidyl-4-(p-maleimidophenyl)butyrate (SMPB), iodoacetate, succinimidyl-4-(N-maleimidomethyl)cyclohexane-1-carboxylate (SMCC).

Two general methods are used to prepare immunogenic (i) SAg-GalCer, (ii) GalCerGal, (iii) GalCerGal-SAg and (iv) SAg-glycosphingolipid complexes: The molecules (1) are dissolved in solution and encapsulated within the vesicle construction, or (2) covalently coupled to phospholipid constituents in the lipids using standard cross-linking chemical reactions. Covalent coupling of SAg to liposomes is done through the head groups using various phospholipid derivatives and cross-linking chemical reactions. These are done via the PE molecules. Simple encapsulation is also a viable technique as described in Hermanson (supra).

A sample method using periodate oxidation and reductive amination is given below.

1. A 5 mg/ml liposome suspension is prepared in 20 mM sodium phosphate 0.15 M NaCl, pH 7.4. containing, on a molar ratio basis as mixture of phosphatidyl choline: cholesterol:phosphatidyl glycerol of 8:10:1. Other liposome compositions may be used, for example methods without cholesterol, as long as a periodate-oxidizable component containing vicinal hydroxyls (e.g., phosphatidyl glycerol) is present. Any method of liposome formation may be used that is common to those skilled in the art including mechanical dispersion.

2. Sodium periodate is dissolved to a concentration of 0.6 M by adding 128 mg/ml of water. 200 ml of this stock periodate solution is added to each mol of the liposome suspension with stirring.

3. React for 30 min. at room temperature in the dark.

4. The oxidized liposomes are dialyzed against 20 mM sodium borate, 0.15 M NaCl, pH 8.4, to remove unreacted periodate. This buffer is ideal for the subsequent coupling reaction. Chromatographic purification using a column of Sephadex G50 is also done. The periodate-oxidized liposomes are used immediately to couple with SAg molecules or they may be stored in a lyophilized state in the presence of sorbitol for later use.

5. SAg is added to the periodate oxidized liposome solution to obtain a 1 mg/ml concentration.

6. In a fume hood, add 20 ml of 5 M sodium cyanoborohydride solution in 1 M NaOH (Aldrich) to each ml of the SAg solution.

7. The reaction is mixed gently and incubated at room temperature for 6 hours.

8. Excess SAg and cyanoborohydride are removed by size exclusion chromatography on a column of Sephadex G-50 or by dialysis using a membrane with a molecular weight cutoff of 100,000 daltons.

9. Ganglioside antigens isolated by the method described previously are incorporated into SAg-containing liposomes by detergent dialysis. An amount of ganglioside is added representing twice the amount of phosphatidyl glycerol (on a molar basis) originally added to form the liposome (prior to periodate oxidation). To this solution, concentrated sodium deoxycholate is added to obtain a final concentration of 0.7% (w/w) and mixed thoroughly using a Vortex mixer. Finally, the liposome suspension is dialyzed against PBS, pH 7.5. A sample of the encapsulation technique is given in Hermanson, supra.

An additional method for preparation of liposomes containing GalCer or glycosphingolipids with terminal Gal(α 1-4)Gal is as follows: The donor liposomes consist of liver phosphatidylcholine, dicetyl phosphate, cholesterol, 3-(Man1-3Man-sn-1,2diacylglycerol) and galactosylceramide. These are mixed in various percentages to permit optimal expression of the galactosylceramide. Constituent lipids in chloroform-methanol are mixed and dried under a stream of nitrogen. Buffer consisting of 0.15M NaCl, 10 MM sodium Phosphate, pH 7.4, 1 mM dithiothreitol, 0.02% NaN3 is added to the dried lipids at a volume of 1 ml per 0.9 mmol of lipid phosphorus in the donor liposomes. After a 30-min incubation at 25° C., the lipids are dispersed into the buffer by sonication with a Bransom sonifier for 30 min under nitrogen at 0° C. The liposome suspension is used the same day after centrifugation at 1500 g for 30 min to remove any undispersed lipid and titanium fragments released from the sonication probe.

Liposomes used for transfer of nucleic acid constructs given herein have unique structures as described below. A cationic liposome composed of dimyristyloxypropyl-3-dimethyl-hydroxyl ammonium (DMRIE) with DOPE has allowed up to 100 fold higher concentrations of lipid and DNA to be administered in vivo with minimal toxicity. Improved transfection techniques have been observed with the DMRIE/DOPE of two to seven fold. The prototype cationic lipid for gene transfer is DOTMA (N[1-(2,3-dioleyloxy)propyl]-N,N,N-tri-methylammonium chloride) which is mixed with a equimolar amount of DOPE (dioleoyl phosphatidylethanolamine). The lipid DOTMA/DOPE comprise the cationic liposome known a Lipofectin. For human studies, two different cationic liposomes formulations are used. The first includes DC-cholesterol (3β[N—(N′N′-dimethylaminoethane)-carbamoyl]cholesterol) mixed with DOPE. DC-cholesterol/DOPE is low concentrations has proven to reduce toxicity to cells in vitro, is metabolized in vivo, and has provided successful gene transfer into malignant tumors in humans (See Example 17 for use in humans).

Genetic Fusion of SAgs to LPS's

N-linked glycosylation occurs exclusively in the ER, where Glc3Man9GlcNAc2 is added to Asn residues present in the sequence Asn X Ser/Thr (X, any residue except Pro). To produce a glycosylation site on a SAg capable of binding a LPS, recombinant vaccinia virus expressing SAg is produced with Gln149 or Asn149 directed to the ER by appendage of NH2-terminal ER insertion, The SAg is directed to the secretory pathway using signal sequence from IFNβ. Recombinant vaccinia viruses(rVVs) expressing TAP and SAg nucleoprotein are used. The full length SAg gene modified by standard molecular genetic methods to encode glycosylation sites is inserted into the thymidine kinase locus of vaccinia viruses (VVs) by homologous recombination as described using the pSX11 plasmid to express foreign proteins under the control of the VV p7.5 early/late promoter. SAg nucleoprotein is directed to the secretory pathway using the signal sequence from IFNβ. The SAg coding sequences of all of the rVVs are verified by sequencing PCR-amplified copies of full-length NP genes isolated from the rVV. The resulting SAg-LPS or SAg-lipoprotein complexes are used to immunize a population of T or NKT effector cells for use in the adoptive immunotherapy of cancer (Examples 2, 5, 7 15, 16, 18-23). They may be preloaded onto CD1 or MHC Class I or II receptors on APCs as described below in the course of ex vivo immunization. These complexes may also be used in vivo as a preventative or therapeutic antitumor vaccine as in Example 14, 15, 16, 18-23).

Preparation of Fusion Proteins

Preferred fusion proteins comprise SAgs linked to other proteins or peptides such as VTs or their A and B subunits, IFNα receptors, CD19 peptides or carbohydrate recognition units which are designed to target the SAg to glycosphingolipid receptors on tumor cells or αᵥβ₃ ligand Arg-Gly-Asp or αᵥβ₅ ligand Asn-Gly-Arg in vivo or in vitro. These fusion proteins induce apoptosis of the tumor cells. The fusion proteins are produced by conventional methods in a variety of cells using a variety of vectors such as phage 1 regulatory sequences. Techniques are well established for producing fusion proteins that include the lacZ protein(β-galactosidase), trpE protein, glutathione-S-transferase, and thioredoxin. Expression in E. coli is most conventional but baculoviral expression systems are also useful. Fusion proteins are produced in bacteria by placing a strong, regulated promoter and an efficient ribosome-binding site upstream of the cloned gene. Exemplified below is a procedure using a representative lacZ vector. However, it should be recognized that other vectors well known in the art would be useful. Plasmids encoding the above proteins are prepared as previously described.

Construction of Expression Plasmids and Detection of Fusion Proteins

1. The appropriate pUR (or pEX or pMR100) vector is ligated in-frame to cDNA fragments to be expressed as fusion partners using the above plasmids to create an in-frame fusion. cDNA encoding the verotoxins may be obtained from Dr. G. Lingwood, University of Toronto; murine p31 Ii are from Dr. R. Germain, National Institutes of Health and J. Miller, University of Chicago.

2. Bacteria of the following strains are transformed: E. coli K12 71/18 or JM1O3 with pUR vectors, M5219 with pEX vectors or LG9O for pMR100 vectors. The cells are plated on LB medium containing ampicillin (100 mg/ml) and incubated overnight at 37° C. (or 30° C. in the case of the pEX vector). MacConkey lactose indicator plates should be used for pMR100.

3. Individual colonies are tested for the presence of the desired insert by plasmid minipreps.

If most of the colonies can be assumed to contain a cDNA (because directional cloning or a dephosphorylated vector was used in step 1), they can be screened for protein production in parallel (see step 4b). If not, clones that contain a cDNA, as determined by plasmid minipreps, can be screened for protein expression later. cDNA inserts into a pMR100 plasmid can be detected readily as red colonies on the MacConkey lactose indicator plates.

4. Colonies are screened as follows for expression of the fusion protein.

a. Grow small cultures from 5-10 colonies in LB medium containing ampicillin (100 mg/ml). Incubate overnight at 37° C. (or at 30° C. for pEX).

b. Inoculate 5 ml of LB medium containing ampicillin (100 mg/ml) with 50 ml of each overnight culture. Incubate for 2 hours at 37° C. (or at 30° C. for pEX) with aeration. Remove 1 ml of uninduced culture, place it in a microfuge tube, and process as described in steps d and e. If screening for protein production is being done in parallel, prepare plasmid minipreps from 1-ml aliquots of the overnight cultures.

c. Induce each culture as follows: For pUR or pMR100 vectors, add isopropylthio-b-D-galactoside (IPTG) to a final concentration of 1 nM and continue incubation at 37° C. with aeration. For pEX vectors, transfer the culture to 40° C. and continue incubating with aeration.

d. At various time points during the incubation (i.e., 1, 2, 3, and 4 hours), transfer 1 ml of each culture to a microfuge tube, and centrifuge at 12,000 g for 1 minute at room temperature in a microfuge. Remove the supernatant by aspiration. The kinetics of induction varies with different proteins, so it is necessary to determine the time at which the maximum amount of product is produced.

e. Resuspend each pellet in 100 ml of 1×SDS gel-loading buffer, heat to 100° C. for 3 minutes, and then centrifuge at 12,000 g for 1 minute at room temperature. Load 15 ml of each suspension on a 6% SDS polyacrylamide gel. Use suspensions of cells containing the vector alone as a control. (For pEX and ORF vectors, also use b-galactosidase as a control.) The fusion protein should appear as a novel band migrating more slowly than the intense b-galactosidase band in the control. It is not uncommon for a protein the size of b-galactosidase to be present along with the fusion protein.

Composition of 1×SDS Gel-Loading Buffer

50 mM Tris Cl (pH 6.8)

100 mM dithiothreitol (DTT)

2% SDS (electrophoresis grade)

0.1% bromophenol blue

10% glycerol

1×SDS gel-loading buffer lacking dithiothreitol can be stored at room temperature. Dithiothreitol should then be added, just before the buffer is used, from a 1 M stock.

Loading of SAg-LPS, SAg-Lipoprotein, SAg-Lipid, SAg-Glycolipid or SAg-Phytolipid Conjugates onto CD1 or MHC Receptors

For loading of SAg LPS or SAg-lipoprotein SAg-lipid, SAg-glycolipid SAg-phytolipid conjugates or SAg-lipid complexes from section 55 onto CD1 receptors, recombinant soluble CD1-2M complexes in Drosophila melanogaster cells are used to screen a random peptide phage display library(RPPDL). The absence of peptide-loading machinery in D. melanogaster cells results in the expression of class 1 molecules that are properly folded and functionally competent but essentially devoid of bound peptide. This approach has been shown to be useful in defining peptide binding motifs for classical and nonclassical MHC Class I and Class II molecules. (Jackson et al, Proc. Natl. Acad Sci. 89:1217-1224, 1992; Hammer et al, J. Exp. Med 175,1007-1012, 1992; Hammer et al, Cell 74, 197-201, 1993). Each clone of SAg-lipoprotein contains a random 22-amino acid sequence at the mature NH2 terminus of the gene VIII (filamentous coat protein of the M13 bacteriophage). Recombinant soluble mCD1 is engineered with a C-terminal hemagglutinin (HA) tag, an epitope derived from the influenza HA protein. In this way, the mCD1-phage complexes are identified with a HA tag specific antibody. For immunizing usage, isolated receptor or antigen presenting cells of various types which express CD1 or MHC class II molecule pretreated with formaldehyde may be used for loading the SAg-LPS or SAg-lipoprotein complexes. These APCs with bound complexes are then used to immunize T cells or NKT cells for use in adoptive immunotherapy of cancer (Examples 2, 7, 15, 16 18-23).

Incorporation of Exogenous Lipid e.g. Glycolipid, Phytosphingosine, Apolipoprotein or oxyLDL into Cells by Fusion with Liposomes

Liposomes

To prepare glycolipid, phytosphingosine, apolipoprotein, oxyLDL or receptor containing liposomes, 400 mg of galabiosylceramide (Gb2) globotriosylceramide (Gb3), globotetraosylceramide (Gb4), galactosylceramide (GalCer),glucosylceramide (GlcCer), phytoshpingosine, oxyLDL or apolipoprotein are dried with 200 mg of phosphatidylethanolamine (PE) and 200 mg of phosphatidylserine (PS) under a stream of nitrogen gas. 400 ml of sterile isotonic PBS, pH 7.4, is added to the lipid, and the mixture is sonicated using a water bath sonicator for 30 minutes. Liposome preparations are used immediately.

To incorporate exogenous glycolipid into cells, tumor cells in late logarithmic growth phase, sickled erythrocytes or vesicles (1.6×10⁷ cells) are washed twice with PBS to remove serum proteins and then suspended in serum-free RPMI 1640 medium at 4×10⁶ cells/ml. The cells are incubated in the presence of the liposomes (or PBS for controls) prepared as above with rotary shaking (100 rpm) at 37° C. for 1 hr., washed twice (5 min. 800×g) with PBS. and incubated for 18-24 hr at 37° C. in the presence of medium supplemented with 10% fetal calf serum prior to use.

Membrane & Vesicle Transfers

Exogenous lipids e.g., galactosylceramides from proximal tubular epithelial cells, MDCK canine renal cells or phytosphinosine from amphibian cells are incorporated into dendritic cells or tumor cells or dendritic cell (accessory cell)/tumor cell fusions as follows:

Preparation of Plasma Membrane

Plasma membrane fractions are prepared and analyzed as described (Monneron A et al., J Cell Biol. 77: 211-231 (1978)). Only the lightest fractions (band 1 at the interface between buffer and 22.5% sucrose and band 2 at the interface between 22.5% and 35% sucrose), containing pure plasma membrane vesicles, are collected and pooled. From 10¹⁰ cells, 0.8-1.0 mg of protein is recovered in the combined fraction. The plasma membrane enzyme markers 5′-nucleotidase (EC 3.1.3.5) and alkaline phosphatase (EC 3.1.3.1) are enriched 30-fold and 70-fold, respectively, over the homogenate; no activity of the cytoplasmic enzyme lactate dehydrogenase are found. No traces of RNA or DNA contamination are found. The membranes obtained are either immediately frozen in aliquots at −70° C. or used at once.

Reconstituted Vesicles

Three types of vesicles are prepared as follows: (i) reconstitution of solubilized Sendai virus envelopes (V vesicles), (ii) reconstitution of solubilized plasma membranes (PM vesicles), and (iii) coreconstitution of solubilized Sendai virus envelope and plasma membranes (VPM vesicles). Sendai virus (20 mg of protein), solubilized in 2 ml of resolution buffer containing 2% Triton X-100 for 1 hr at room temperature, is centrifuged (1 hr. 100,000×g) to yield a clear supernatant (1.5 mg of protein per ml) containing mainly the two envelope glycoproteins, neuraminidase/hemagglutinin glycoprotein (NH) and fusion glycoprotein (F). For preparation of VPM vesicles, freshly solubilized plasma membranes (1 mg of protein per 0.3 ml of resolution buffer containing 1% Triton X-100, 20 min, 4° C.) are mixed with the solubilized viral envelopes (1 mg of protein per 0.7 ml of resolution buffer) and dialyzed in Spectrapor membrane-2 against 1000-fold excess of reconstitution buffer containing 1.5 g of wet Bio-beads SM-2 per liter [for efficient removal of the detergent]. PM and V vesicles are prepared by the same procedure except that one of the components was omitted and replaced by resolution buffer alone. Dialysis is continued for 96 hr at 4° C. until the Triton X-100 concentration dropped below 0.02%. The dialyzed solutions are centrifuged at 100.000×g for 3 hr at 4° C. to obtain reconstituted vesicles. The pellet is suspended in 0.3-0.5 ml of resolution buffer, divided into small aliquots, and frozen at −70° C. Each aliquot is thawed only once for fusion experiments.

Fusion of Reconstituted Vesicles with Tumor Cells

Tumor cells (10⁶ cells) are incubated with 5-30 mg of protein of reconstituted vesicles in 1 ml of fusion solution. After 60 mm at 4° C. with occasional shaking, during which the Sendai virus bound to the cells, the cells are collected by centrifugation, resuspended in 1 ml of fusion solution containing 5 mM Ca²+, and incubated at 37° C. for 30 mm for the fusion process. The cells are then washed with phosphate-buffered saline (pH 7.4) and suspended in RPMI-1640 medium containing 5% (vol/vol) heat-inactivated fetal calf serum

Assessment of Incorporation

Tumor cells are analyzed for uptake of the various glycolipids, apolipoproteins, oxyLDL, phytosphingosine, glycosylceramides by use of direct staining with specific antisera, binding by lectin receptors and they are tested in vitro for activation of T cells, NK cells, NKT cells using proliferative assays and induction of cytokines IL-2, IL-4, IL-12 and IFN-g by methods well established in the art.

### EXAMPLE 6

Targeting SAg Nucleic Acids Phage Display Systems and Polypeptides to Tumor Sites

Parenterally administered nucleic acid is targeted to a particular cell population as follows. Nucleic acid is attached to a desialylated galactose moiety that targets asialo-orosomucoid receptors in liver cells. Nucleic acid is attached to other ligands such as transferrin and TAP-1 as well as antibodies to surface structures such as the Le^{y} receptor. These ligands and antibodies bind to surface structures and are internalized. Thus, the attached nucleic acid is delivered to a cell of choice.

Sickled Erythrocytes as Gene Carriers

Erythrocytes from patients with sickle cell anemia contain a high percentage of SS hemoglobin which under conditions of deoxygenation aggregate followed by the growth and alignment of fibers transforming the cell into a classic sickle shape. Retardation of the transit time of sickled erythrocytes results in vaso-occlusion. SS red blood cells have an adherent surface and attach more readily than normal cells to monolayers of cultured tumor endothelial cells. Reticulocytes from patients with SS disease have on their surface the integrin complex α₄bβ₁ which binds to both fibronectin and VCAM-1, a molecule expressed on the surface of tumor endothelial cells particularly after activation by inflammatory cytokines such as TNF, interleukins and lipid-mediated agonists (prostacyclins). Activated tumor endothelial cells are typically procoagulant. Similar molecules are upregulated on the neovasculature of tumors. In addition, upregulation of the adhesive and hemostatic properties of tumor endothelial cells are induced by viruses, such as herpes virus and Sendai virus. Sickled erythrocytes lack structural malleability and aggregate in the small tortuous microvasculature and sinusoids of tumors. In addition, the relative hypoxemia of the interior of tumors induces aggregation of sickled erythrocytes in tumor microvasculature. Hence, sickled erythrocytes with their proclivity to aggregate and bind to the tumor endothelium are ideal carriers of therapeutic genes to tumor cells.

Red blood cell mediated transfection is used to introduce various nucleic acids into the sickled erythrocytes. The extremely plastic structure of the erythrocyte and the ability to remove its cytoplasmic contents and reseal the plasma membranes enable the entrapment of different macromolecules within the so-called hemoglobin free “ghost.” Combining these ghosts and a fusogen such as polyethylene glycol has permitted the introduction of a variety of macromolecules into mammalian cells (Wiberg, F C et al, Nucleic Acid Res. 11: 7287-7289 (1983); Wiberg, F C et al., Mol. Cell. Biol. 6: 653-658 (1986); Wiberg, F C et al., Exp. Cell. Res. 173: 218-227 (1987). Both transient and stable expression of introduced DNA are achieved by this method. Sickled cells can also be transfected with a nucleic acid of choice e.g., apolipoproteins, RGD in the nucleated prereticulocyte phase (e.g.proerythroblast or normoblast stage) by methods given in Example 1. Sickled erythrocytes transfected with nucleic acids encoding a SAg and/or carbohydrate modifying enzyme to induce expression of the a α-Gal epitope, apolipoproteins, RGD and/or any construct described herein. Nucleic acids encoding additional polypeptides alone or together with SAg as described in Tables I and II to including but not limited to angiostatin, apolipoproteins, RGD, streptococcal or staphylococcal hyaluronidase, chemokines, chemoattractants and Staphylococcal protein A are transfected into and expressed by sickled erythrocytes. These sicled cell transfectants are administered parenterally and localize to tumor neovascular endothelial sites where they induce a anti-tumor response. The methods of in vivo transfection of tumor cells are given in the Examples 17. Protocols for use of these transfectants in the induction of anti-tumor immune response are described in Examples 14, 15, 16, 18-23, 31

Vesicles from Sickled Erythrocytes

Vesicles from sickled erythrocytes are shed from the parent cells. The contain membrane phospholipids which are similar to the parent cells but are depleted of spectrin. They also demonstrate that a shortened Russell's viper venom clotting time by 55% to 70% of control values and become more rigid under acid pH conditions. Rigid sickle cell vesicles induce hypercoagulability, are unable to pass through the splenic circulation from which they are rapidly removed. Sickled erythrocytes are transfected in the nucleated prereticulocyte phase with superantigen and apolipoprotein nucleic acids as well as RGD nucleic acids. Nucleic acids encoding additional polypeptides alone or together with SAg as described in Tables I and II are transfected into and expressed by sickled erythrocytes. Any of the the immature or mature sickled erythrocytes and their shed vesicles expressing the molecules given in Tables I and II are capable of localizing to tumor microvascular sites where they bind to apolipoprotein receptors and induce an anti-tumor effect. Because of their adhesive and hypercoagulable properties as well as their rigid structure, these sickled cell vesicles expressing superantigen and apolipoproteins are especially useful for targeting the tumor microvascular endothelium and producing a prothrombotic, inflammatory anti tumor effect. Sickled erythrocytes and their vesicles are capable of acquiring oxyLDL via fusion with oxyLDL containing liposomes as in Example 5. The resulting sickle cell or liposome expresses oxyLDL alone or together with SAg. Binding of oxyLDL to the SREC receptor on tumor microvascular endothelial cells induces apoptosis and simultaneous superantigen deposition produces a potent T cell anti-tumor effect.

Vesicles are prepared and isolated as follows: Blood is obtained from patients with homozygous sickle cell anaemia. The PCV range is 20-30%, reticulocyte range is 8-27%, fetal hemoglobin range is 25-13% and endogenous level of ISCs is 2-8%. Blood is collected in heparin and the red cells are separated by centrifugation and washed three times with 09% saline. Cells are incubated at 37° C. and 10% PCV in Krebs-Ringer solutions in which the normal bicarbonate buffer is replaced by 20 mM Hepes-NaOH buffer and which contains either 1 mM CaCl2 or 1 mM EGTA. All solutions contain penicillin (200 u/mI) and streptomycin sulphate (100 ug/mI). Control samples of normal erythrocytes are incubated in parallel with the sickle cells. Incubations of 10 ml aliquots are conducted in either 100% N2 or in room air for various periods in a shaking water bath (100 oscillations per mm). N2 overlaying is obtained by allowing specimens to equilibrate for 45 mm in a sealed glove box (Gallenkamp) which was flushed with 100% N2. Residual oxygen tension in the sealed box was less than 1 mmHg. The percentage of irreversibly sickled cells is determined by counting. 1000 cells after oxygenation in room air for 30 mm and fixation in buffered saline (130 mM Cl, 20 mM sodium phosphate, pH 74) containing 2% glutaraldehyde. Cells whose length is greater than twice the width and which possessed one or more pointed extremities under oxygenated conditions are considered to be irreversibly sickled. After various periods of incubation, cells are sedimented at 500 g for 5 mm and microvesicles) are isolated from the supernatant solution by centrifugation at 15,000 g for 15 mm. The microvesicles form a firm bright red pellet sometimes overlain by a pink, flocculent pellet of ghosts (in those cases where lysis was evident) which is removed by aspiration. Quantitation of microvesicles is achieved by resuspension of the red pellet in 1 ml of 05% Triton X100 followed by measurement of the optical density of the clear solution at 550 nm. Optical density measurements at 550 nm give results that are relatively the same as measurements of phospholipid and cholesterol content in the microvesicles. Cell lysis is determined by measurement of the optical density at 550 nm of the clear supernatant solution remaining after sedimentation of the microvesicles. Larger samples of microvesicles for biochemical and morphological analysis are prepared from both sickle and normal cells following incubation of up to 100 ml of cell suspension at 37° C. for 24 h in the absence or presence of Ca²⁺. Ghosts are prepared from sickle cells after various periods of incubation. The cells are lysed and the ghosts washed in 10 mM Tris HCl buffer, pH 73, containing 02 mM EGTA.

These vesicles are useful as a preventative or therapeutic vaccine as in Examples 15, 16, 18-23, 36.

Phage Displayed SAgs

Phages displaying or free tumor homing peptides ligands such as the tripeptides Arg-Gly-Asp and Asn-Gly-Arg which tripeptides bind to the integrins avb3 and avb5, respectively, that are located on tumor microvasculature, are conjugated to (1) a SAg peptide, (2) naked DNA encoding a SAg peptide or (3) phage displaying a SAg peptide. These constructs are prepared as in Examples 3 and 5 and are further described in Jackson R H. et al., In: Protein Engineering: A Practical Approach, A. R. Rees et al. (eds), pp. 277-301, Oxford Press, London, 1992. Similarly tumor cells or sickled cells transfected with and expressing SAgs and other molecules given in Tables I and II are also transfected with nucleic acids encoding RGD which facilitates their localization to tumor microvasculature. These conjugates or transfectants are administered i.v. and localize to the tripeptides' integrin receptors situated on the tumor microvasculature. Neovascular endothelial cells to which these constructs have been targeted are transfected by SAg-encoding DNA so that they express or secrete SAgs locally. This induces potent local T cell activation and engender a tumoricidal immune response. Protocols for use of such conjugates, i.e., (1) naked SAg DNA conjugated to the integrin-binding peptides or (2) naked SAg DNA conjugated to phage that display the integrin-binding peptides, and transfectants in the induction of anti-tumor immune response are described in Examples 7, 15, 16, 18-23, 31

Nucleic Acid and Nucleoprotein SAg Mimics

SAgs are often incapable of homing to tumor cells expressing SAg receptors in vivo because of the existence of naturally occurring SAg-specific antibodies and the affinity of SAgs for class II receptors on a wide variety of cells. To solve this problem, DNA chromatography is used to identify oligonucleotides instead of SAg peptides that bind to SAg receptors which are naturally expressed on tumor cells. The SAg receptor-specific oligonucleotides are conjugated to a SAg peptide with a functional TCR or NKT cell binding site. Oligonucleotides are also substituted for peptides in the SAg molecule which bind to MHC class receptors and naturally occurring SAg-specific antibodies. These conjugates are used to target SAgs to tumor cells in vivo that either endogenously express a SAg receptor or are pre-transfected with nucleic acid encoding a SAg receptor. These peptide-oligonucleotide complexes are prepared by chemical conjugation methods well known in the art. Such receptor specific oligonucleotides may have several fold greater affinity for the SAg receptor compared to the native SAg. While these peptide-oligonucleotide complexes are used predominantly in vivo to target tumor cells bearing SAg receptors, they are also used ex vivo to stimulate T cells to become tumor specific effector cell which are useful for adoptive immunotherapy of cancer (Example 7, 15, 16, 18-23).

In appropriate recombinant bacteria, nucleic acids encoding the SAg receptor binding site expressed on tumor cells are fused to nucleic acids encoding SAgs. The resultant SAg polypeptide construct consists of the amino acid sequence of a SAg and its SAg receptor binding site (which is overexpressed if desired). The SAg with its expressed or overexpressed SAg binding site is useful in targeting tumor cells expressing SAg a receptors after administration to a tumor-bearing host.

In a related construct, the nucleic acid encoding a SAg with an overexpressed SAg receptor specific binding site is fused to the nucleic acid encoding a native or chimeric SAg with its binding site for naturally occurring antibodies and its MHC class II binding site removed, mutated or replaced by peptides from another SAg against which there are no known naturally occurring antibodies. The TCR binding and activating region of this molecule is conserved. This resulting SAg polypeptide molecule binds to SAg receptors on tumor cells but also retains its capacity to activate the TCR. It is administered parenterally or orally to a tumor bearing host (orally to a colon carcinoma patient) and will effectively target tumor cells with SAg receptors (such as colon carcinoma cells) without being diverted by naturally occurring antibodies or class II receptor bearing cells present in whole blood. As such, this construct is useful in producing an anti-tumor effect when administered to a tumor bearing host as in Example 18-23).

Using DNA chromatography techniques, nucleic acid specific for SAg receptors on tumor cells are identified. These nucleotides are conjugated to SAg polypeptides which are optionally devoid of class II binding sites and naturally occurring antibody binding sites but with conserved TCR binding and activating sites. These constructs are useful in targeting tumor cells bearing SAg receptors in vivo while retaining SAg amino acid sequences specific for the TCR which are capable of producing a tumor specific T cell population effective in adoptive immunotherapy of cancer. The selected amino acid sequences are deleted, replaced or added to the SAg molecules using molecular cloning and site directed mutagenesis techniques well established in the art.

### EXAMPLE 7

General Ex Vivo Immunization Methods to Produce Tumor Specific Effector Cells for Adoptive Immunotherapy of Cancer

Several days (3 to 60 days) after intratumoral immunization with a nucleic acid construct described herein, tumor draining lymph nodes are removed and placed in tissue culture. These cells are further expanded in vitro with SAg polypeptide for 2-4 days and/or IL-2 in vitro for a total of 3-15 days. These T cells are then harvested and reinfused into the host. T effector cells produced after in vivo immunization with nucleic acid encoding a SAg are expected to display potent anti-tumor activity.

Cells transfected ex vivo, are administered to the host wherein they activate lymphocytes in a number of ways. In one embodiment, the initial step involves in vivo immunization of hosts using various transfectants and constructs as described in Table II. The transfected cells are introduced into the host tumor, a nearby region, subcutaneously in close proximity to regional lymph nodes, or the lymph nodes draining the tumor. Transfected cells types, constructs and agents used in this step are given in Table II. Tumor cells are irradiated or treated with mitomycin C after transfection with nucleic acid encoding a SAg and/or another polypeptide so that polypeptides are expressed and fixed on the cell surface and the tumor cells do not proliferate when administered to the host.

In another embodiment, the initial step involves in vivo immunization of the tumor bearing host with transfectants, constructs and cells as described in Table III. These agents are administered in close proximity to the regional lymph nodes with or without a bacterial adjuvant such as bacillus Calmette-Guerin (BCG) or Corynebacterium parvum. The lymph node cells are harvested 10 days later and tissue cultured for further in vitro immunization/stimulation with SAg or SAg expressing cells that, optionally, coexpress a tumor associated antigen, costimulatory molecule or antigen presenting molecule.

Cryopreserved autologous tumor cells for subsequent tumor vaccination and culture are obtained from patients. Fresh resected tumors are dissociated under sterile conditions into single cell suspensions by mechanically mincing tumor into 5-mm3 pieces followed by enzymatic digestion. Generally, 1 gm of tumor is digested in a minimum volume of 40 ml of an enzyme mixture consisting of Hank's balanced salt solution (HBSS) containing 2.5 units/ml of hyaluronidase type V, 0.5 mg/ml of collagenase type IV, and 0.05 mg/ml of deoxyribonuclease type I (all commercially available from Sigma Chemical Co.; St. Louis, Mo.). The digestion is performed at room temperature with constant stirring in a trypsinizing flask for 2 to 6 hours.

The resulting cell suspension is filtered through a layer of No.100 nylon mesh (Nytek: TETKO, Inc.; Briarcliff Manor, N.Y.) and cryopreserved in 90% human AB serum (GIBCO; Grand Island, N.Y.) plus 10% dimethyl sulfoxide (Sigma) at −178° C. in liquid nitrogen for subsequent immunization and culture.

Tumor cells are used in native form, with dinitrophenyl (DNP) or other haptens conjugated to them and then irradiated or treated with cytostatic drugs prior to use. Optionally, the tumor cells are transfected with nucleic acid encoding a SAg, and/or tumor associated antigen, and/or antigen presenting molecule, and/or costimulatory molecule, and/or adhesion molecule, and/or xenogeneic antigen, and/or carbohydrate modifying enzyme. The nucleic acid is introduced by methods given previously. The cells are then irradiated to a dose of 25 Gy or treated or with cytostatic drugs, viable cells counted by trypan blue exclusion and the cells resuspended so that a volume of 0.2 to 0.4 ml contains 1-2×10⁷ with or without⁷ colony forming units of fresh frozen TICE BCG.

Patients are vaccinated intradermally (i.d.) at two sites approximately 10 cm from superficial inguinal lymph nodes. If necessary, axillary lymph nodes are used. Lymph node regions with previous dissections or clinical evidence of tumor are avoided.

Accessory cells including DCs, fibroblasts, endothelial cells, monocytes, and macrophages are used after transfection with nucleic acid encoding a tumor associated antigen, and/or SAg, and/or xenogeneic antigen, and/or carbohydrate modifying enzyme.

If desired, these accessory cells or APCs are transfected with recombinant viral vectors containing nucleic acid the encode a SAg, and/or tumor associated antigen, and/or costimulatory molecule, and/or antigen presenting molecule, and/or costimulatory molecule, and/or adhesion molecule, and/or xenogeneic antigen. These cells need not be irradiated prior to administration. These cells are administered using the same cell numbers given above with or without BCG.

Alternatively, patients are vaccinated with various tumor associated antigens and other agents as described in Table II. The agents are bound to MHC class I, class II or CD1 receptors or to cells expressing these receptors. They are also given alone in doses ranging from 0.1 to 10 mg emulsified in various adjuvants well described in the art. A vaccination course includes up to 6 inoculations of the above agents at 1-3 week intervals.

Table III

Single Step in vivo Immunization of Tumor Bearing Hosts with SAg Nucleic Acids Alone, Combined with Nucleic Acid Encoding Other Peptides and SAg Nucleic acids Conjugated to Polypeptides or Liposomes

I. Intratumoral injection of nucleic acid

1. Direct injection of SAg nucleic acids into tumor.

2. Direct i.v. or intra-arterial injection of SAg nucleic acids into tumor microvasculature.

a. SAg nucleic acids conjugated to a polypeptide ligand specific for a tumor cell, tumor stromal cell, tumor microvascular or neovascular cell receptors

b. Nucleic acid within liposomes containing a monoclonal antibody.

3. Recombinant viruses containing nucleic acid.

a. Inactivate the virus in the host with gancyclovir

II. After in vivo immunization (3-14 days), harvest regional lymph nodes and place in tissue culture.

III. Activate and expand lymphocytes.

1. Treat with SAg for 2 days.

2. Treat with IL-2 for 3 days.

IV. Inject tumor specific effector T cells into host.

Regional lymph node cells draining tumor sites, lymphoid cells obtained after the above priming, peripheral blood T cells, and tumor infiltrating lymphocytes (TILs) are suitable sources of T cells that are activated to function as effector cells (T cells activated against the cancer cells). T cells are obtained from tumor infiltrating lymphocytes either before or after tumor vaccine immunization in vivo by the methods described herein. Approximately 10 days after in vivo immunization, an enlarged draining lymph node is removed and cultured. An immunized lymph node used herein is exemplary. A single cell suspension of lymph node cells is obtained by mechanical dissociation. Briefly, lymph nodes are minced into 2 mm³ pieces in cold HBSS with a scalpel. The fragments are then pressed through a stainless steel mesh with a glass syringe plunger. The resultant cell suspension is filtered through nylon mesh and washed in HBSS. Cultures are established in 300-ml culture bags (Livecell Flasks; Fenwal, Deerfield, Ill.) with 200 to 250 ml of culture medium (CM: RPMI 1640 with 10% human AB serum, 2 mM fresh L-glutamine, 1 mM sodium pyruvate, 100 mg/ml of streptomycin, and 50 mg/ml of gentamicin all from GIBCO; Grand Island, N.Y.), containing 1-2×10⁵ lymph node cells/ml and 1-4×10⁵ irradiated (60 Gy) tumor cells/ml. Optionally, the lymph node cells are further separated into populations CD4+CD8+ T cells, NKT cells and /+ T cells. Some SAg complexes are presented bound to MHC class II receptors and some such as SAg-LPS complexes or SAg-glycosylceramide complexes are presented bound to CD1 receptors either free or on APC cell surfaces.

After 24 hours, various SAgs or SAg transfected cell types (STCT) given in Table III are added in doses of 10⁵ to 10⁷ cells for 8-72 hours. The cells are harvested and used for in vivo administration at this point. Specific cell populations are selected such as those having a particular TCR V profile or expressing CD44 using magnetic beads or other separation techniques well known in the art. Optionally, the SAg activated T cells are expanded. Recombinant IL-2 (Cetus, Emeryville, Calif.: provided by Cancer Treatment Evaluation Program, National Cancer Institute) is added at the initiation of the cultures at a concentration of 600 IU/ml (1 Cetus unit=6 IU of IL-2). Culture bags are incubated at 37° C. in humidified 5% CO2. Cell counts from aliquots obtained from random bags are followed to observe lymphoid cell proliferation. Lymph node cells are harvested when cells reached maximal density, usually after a total of 5-7 days in culture followed by IL-2 at 24 IU/ml for 3 days. These intervals are shortened depending on the cell viability, CD44 expression, or V expression or other conditions that adversely affect survival, viability, or therapeutic success.

TABLE IV
Two Step in vivo/in vitro Methods and Agents
for Producing Tumor Specific Effector T cells

A. In vivo immunization with SAg transfected tumor cells, accessory
cells, or virus.
1. Tumor cells transfected with:
a. Nucleic acid encoding a SAg
b. Nucleic acid encoding a tumor associated antigen
c. Nucleic acid encoding a carbohydrate modifying enzyme
2. Accessory cells transfected with:
a. Nucleic acid encoding a SAg
b. Nucleic acid encoding a tumor associated antigen
c. Nucleic acid encoding a carbohydrate modifying enzyme
d. Nucleic acid encoding an MHC molecule
3. Recombinant viruses containing:
a. Nucleic acid encoding a SAg
b. Nucleic acid encoding a tumor associated antigen
c. Nucleic acid encoding a carbohydrate modifying enzyme
d. Nucleic acid encoding an MHC molecule
B. *In vivo immunization with:
1. Irradiated tumor cells.
2. Tumor associated antigens.
3. Irradiated tumor cells conjugated with DNP.
4. Tumor associated antigen/SAg conjugate or fusion polypeptides.
5. Naked nucleic or plasmid or phage displayed nucleic acid encoding
a SAg or attached to liposomes or albumin microspheres.
6. Naked or plasmid or phage displayed nucleic acid encoding a
SAg/tumor associated antigen polypeptide conjugate.
7. Tumor cells or accessory cells transfected with nucleic acids
encoding structures given in Table I Group IA, (pages 5 and 6)
GM-CSF, IL-2 and other cytokines. (Berns, AJM. et al., Human Gene
Therapy 6: 347-368 (1995).
8. Tumor cells transfected with nucleic acids encoding chemokines
(T and NKT cell chemoattractants) and granulocyte chemoattractants
(C3a, C5a, MAP).
9. SAg naked DNA fused or in mixture with DNA or structures
non-transfected given in Table 1 IA B and C (pages 5 and 6)
*In vivo immunization may be by various routes, e.g.,, i.d., i.m.,
or as organoids or in adjuvants proximate to regional lymph nodes
e.g., inguinal lymph nodes. For tumor peptide genes an ISS is useful
as is cotransfection of MHC class I genes. For SAg and tumor
associated antigen genes, the ISS is useful.
C. Lymphoid cells from draining lymph nodes are harvested 3-21
days later and placed in tissue culture for further stimulation.
They are divided into T cell, NKT cell and / T cell populations.
Alternatively, T cells, NKT cells and / T cells are obtained from
the peripheral blood and also placed in tissue culture for further
stimulation.
D. In vitro stimulation of T or NKT cell populations to produce
tumor specific effector cells as described in “C” is carried
out with STCT (SAg transfected cell types) or with constructs alone
or applied to appropriate receptors on APCs. MHC class II APCs are
used for presentation of SAg constructs. APCs expressing mannose,
or CD1 or CD14 receptors are used for presentation of glycosylated
SAg, SAg-LPS complexes, SAg-peptidoglycan complexes or SAg-glyco-
sylceramide complexes. Isolated MHC class I, class II, mannose,
CD1 or CD14 receptors immobilized on solid supports such as
polystyrene plates may be used in place of APCs methods well known
in the art. In this form they bind corresponding ligands in the
constructs given above for presentation to T cells or NKT cells.
STCT include tumor cells, accessory cells, antigen presenting cells,
prokaryotic cells, autologous, allogeneic or xenogeneic cells lines
and viruses. Accessory cells include the following: DCs, monocytes,
macrophages, endothelial cells, fibroblasts and NK cells. These
cells are transfected with nucleic acids encoding SAgs in
combination with the nucleic acids given below. These nucleic
acids may include the ISS sequence; SAg genes may be used with or
without the ISS sequence.

Antibodies or Fab fragments having specificity for CTLA-4 are added with or without IL-2 at any point to expand the T cell population and avert apoptosis. The cells are washed once at the end of STCT incubation and before the addition of IL-2 and/or anti-CTLA-4 antibodies.

TABLE V
Ex vivo Modes of Antigen Presentation to T Cells or
NKT Cells to Produce Tumor Specific Effector Cells

A. Tumor Cells, Accessory Cells, Accessory Cell/Tumor Cell Hybrids, e.g., DC/Tumor Cell) Transfected
with:
1. SAg-encoding nucleic acid
2. SAg-encoding nucleic acid and tumor associated antigen nucleic acids (to include arrays of tumor
associated epitopes)
3. SAg nucleic acid and MHC class I or II nucleic acids.
4. SAg-encoding nucleic acid and co-stimulatory nucleic acids.
5. SAg-encoding nucleic acid and adhesion molecule nucleic acids.
6. SAg-encoding nucleic acid and a-galactosyltransferase synthetic nucleic acids or xenogeneic species
specific nucleic acids.
7. SAg-encoding nucleic acid and chemoattractant nucleic acids
8. SAg-encoding nucleic acid and glycosylceramide synthesis nucleic acids
9. SAg nucleic acid and lipopolysaccharide synthesis nucleic acids
10. SAg-encoding nucleic acid and microbial lipoprotein or polysaccharide or peptidoglycan membrane or
capsular synthesis nucleic acids
11. SAg-encoding nucleic acid and SAg receptor nucleic acids
12. SAg-encoding nucleic acid and CD1 receptor synthesis nucleic acids
13. SAg-encoding nucleic acid and CD14 receptor synthesis nucleic acids
14. SAg-encoding nucleic acid and SAg promoter and/or global regulator nucleic acids
15. SAg-encoding nucleic acid and oncogene and/or transcription factor nucleic acids
16. SAg-encoding nucleic acid and angiogenesis factor or receptor nucleic acids
17. SAg-encoding nucleic acid and growth factor receptor nucleic acids
18. SAg-encoding nucleic acid and cell cycle protein nucleic acids
19. SAg-encoding nucleic acid and heat shock protein nucleic acids
20. SAg-encoding nucleic acid and chemokine nucleic acids
21. SAg-encoding nucleic acid and cytokine nucleic acids
22. SAg-encoding nucleic acid and tumor suppressor nucleic acids
23. SAg-encoding nucleic acid and antigen processing and trafficking nucleic acids
B. Additional in vitro Stimulatory Agents (preferred receptor)
1. Tumor peptides (Class I or Class II)
2. Tumor peptide-SAg conjugates or fusion proteins (Class I or Class II).
3. Lipopolysaccharide-SAg conjugate (Class II or CD14)
a. arabinose
b. mycolic acid
c. teichoic acid
d. muramic acid (Staphylococcal cell wall glycoprotein)
e. mannan proteoglycans
f. chondroitin-sulfate
4. Glycosylated SAgs. (Class II or mannose)
5. SAg-glycosylceramide conjugates (class II or CD1)
a. GalCer conjugate
b. Gal conjugate
6. SAg-proteosome conjugates
7. SAg or glycosylated SAg or SAg-glycosylceramide conjugates or SAg-lipopolysaccharide or SAg-
peptidoglycan conjugates coupled to proteosomes
8. SAg or glycosylated SAg or SAg-glycosylceramide conjugates or SAg-lipopolysaccharide conjugates or
SAg-peptidoglycan conjugates expressed on or coupled to liposomes
9. Conjugates having having a Superantigen component (polypeptide or nucleic acid) and a partner that is
either a single component or a conjugate of 2 or more components (protein, carbohydrate, lipid DNA) as
indicated below.

| | |
|---|---|
| Superantigen (Protein or DNA) | Partner (Single Component or Conjugate) |
| 1. | DNA coding sequence |
| 2. | Polypeptide |
| 3. | Nucleic acid |
| 4. | Tumor associated Peptide |
| 5. | Tumor Antigen-MHC protein |
| 6. | LPS |
| 7. | Lipoarabinomannan |
| 8. | Ganglioside |
| 9. | Glycosphingolipid |
| 10. | Ganglioside-CD1 receptor |
| 11. | Glycosphingolipid-CD1 receptor |
| 12. | Glycosylceramide (e.g., Gal-Cer) |
| 13. | GalCer-CD1 receptor |
| 14. | Gal |
| 15. | Arg-Gly-Asp or Asn-Gly-Arg |
| 16. | iNOS |
| 17. | Gb2 or Gb3 or Gb4 |
| 18. | (Gb2 or Gb3 or Gb4)-CD1 receptor |
| 19. | -GPI-(Gb2 or Gb3 or Gb4) |
| 20. | -GPI-(Gb2 or Gb3 or Gb4)-CD1 receptor__{—} |
| 21. | Verotoxin |
| 22. | Verotoxin A or B Subunit_{—} |
| 23. | IFNa receptor peptide homologous to VT |
| 24. | CD19 peptide homologous to VT |
| 25. | LDL, VLDL, HDL, IDL |
| 26. | Apolipoproteins (e.g., Lp(a), apoB-100, apoB-48, apoE) |
| | OxyLDL, oxyLDL mimics, (e.g., 7β-hydroperoxycholesterol, |
| | 7β-hydroxycholesterol, 7-ketocholesterol, 5α-6α- |
| | epoxycholesterol, 7β-hydroperoxy-choles-5-en-3β-ol, 4- |
| | hydroxynonenal (4-HNE), 9-HODE, 13-HODE and |
| | cholesterol-9-HODE) |
| 28. | OxyLDL by products (e.g. lysolecithin, |
| | lysophosphatidylcholine, malondialdehyde, 4- |
| | hydroxynonenal) |
| 29. | LDL & oxyLDL receptors (e.g., LDL oxyLDL, acetyl-LDL, |
| | VLDL, LRP, CD36, SREC, LOX-1, macrophage scavenger |
| | receptors) |
| 30. | phytosphingosine, -GPI-phytosphinosine |
| 31. | tumor associated lipid antigens |
| 32. | glycolipid, proteolipid, glycosphingolipid, sphingolipid |
| | with inositolphosphate-containing head groups, |
| | phytoglycolipids, mycoglycolipids, -GPI-sphinosines or |
| | GPI-lipids |
| 33. | sphingolipids with inositolphosphate-containing head |
| | groups having the general structure: |
| | ceramide-P-myoinositol-X with X referring to polar |
| | substituents comprising ceramide-p-inositol-mannose, |
| | inositol-1-P-(6)mannose(α 1,2inositol-1P-(1)ceramide, |
| | (inositol-P)2-ceramide, inositol-P-inositol-P-ceramide, |
| | inositol-P-inositol-P-ceramide. |
| 34. | tumor associated glycan antigens consisting of |
| | peptidoglycans or glycan phosphotidyinositol (GPI) |
| | structures |

C. STCT or SAg-tumor peptide conjugates are incubated with in vivo immunized T cells or NKT cells for 2-4
days and then with IL-2 for 2-5 days.
D. The tumor specific effector cells are then harvested and injected in doses of 10¹⁰-10¹² every 3-7 days for
1-6 treatments.
E. Viruses are transfected into tumor cells, accessory cells, antigen presenting cells, allogeneic or xenogeneic
cells. They are pre-programmed with DNA for SAgs alone or in combination with genes given in D. They
may also utilize the host genome to
produce a new gene product as for example the host -galactosyltransferase. Viruses may include the
following:
1. Adenoviruses.
2. Vaccinia virus.
3. Equine encephalitis virus.
4. Influenza virus.
F. In an additional method, tumor associated antigens are bound to MHC class I positive cells and used to
activate T cells. SAg-lipopolysaccharide complexes and SAg-glycosylceramide complexes are bound to
CD1 or class II receptors on APCs. In addition, SAg-lipopolysaccharide complexes or SAg-
glycosylceramide complexes are presented bound to class II positive APCs. Alternatively, unbound tumor
associated antigen/SAg conjugates or fusion products are added at a 0.1 to 200 mg/ml dose for 2 days. This
is followed by STCT incubation or by native or mutant SAg treatment for 2 days.

For comparative analysis, peripheral blood lymphocytes (PBL) are obtained from patients the same day as the lymph node harvest. PBL are isolated by Ficoll-Hypaque gradients from 60 ml of heparinized blood samples. The PBL are placed in culture utilizing 24-well tissue culture plates at the same cell density as lymph node cells. PBL are harvested at maximal cell density and characterized by phenotype analysis and cytotoxicity. T cells, NKT cells, and NK cells are isolated by well known methods described in the art (Colligan, J E et al., eds, Current Protocols in Immunology, John Wiley, New York, 1996).

PBL are separated by Ficoll/hypaque sedimentation. Cells are recovered from the interface, washed in PBS, and pelleted. Peripheral blood mononuclear cells enriched for MHC class I molecules or MHC class II molecules are used to bind tumor associated antigens or tumor associated antigen/SAg conjugates for in vitro or in vivo immunization.

Cryopreserved groups of autologous PBMCs are thawed, washed twice in PBS, resuspended at 5 to 8×10⁶ cells/ml in CM and pulsed with 1 mg/ml peptide in 15 ml conical tubes (5 ml/tube) for 3 hours at 37° C. These PBMC stimulators are then irradiated at 3000 rads, washed once in PBS, and added to the responder cells at responder stimulator ratios ranging between 1:3 and 1:19.

Tumor infiltrating lymphocytes are isolated from fresh surgical biopsies. Briefly, tumor tissues are minced into 1-mm3 pieces that are then dissociated into single cell suspensions in Dulbecco's modified minimum essential medium (Gibco; Grand Island, N.Y.) supplemented with 10% heat-inactivated human AB serum (NABI, Miami, Fla.), 0.05% collagenase (type 4; Sigma Chemical Co., St. Louis, Mo.), and 0.002% DNase (type 1; Sigma) on a magnetic stirrer for 1 hour. Subsequently, the tissue digests are washed and passed through a nylon mesh and tumor infiltrating lymphocytes and tumor cells are separated on discontinuous (75%/100%) Ficoll/Hypaque gradients.

Lymph node lymphocytes are obtained by mechanical dissociation of tissues, followed by washing in medium and centrifugation on Ficoll/Hypaque gradient [Newell K A, et al., Proc. Natl. Acad. Sci. USA, 88:1074 (1991)]. Cryopreserved suspensions of tumor cells/tumor infiltrating lymphocytes are defrosted, washed, and separated by allowing tumor cells to adhere to the surface of plastic wells. The recovered non-adherent tumor infiltrating lymphocytes are transferred to 6-well plates and cultured in serum-free AJM-V medium (Gibco) supplemented with 6,000 U/ml of IL-2 (Cetus-Chiron, Emeryville, Calif.) for 8 days. Tumor cells are cultured as adherent monolayers in Dulbecco's modified Eagle's medium (DMEM, Gibco) supplemented with 10% (v/v) of fetal calf serum. Any activated lymphocytes can be used in the method given above. In a preferred embodiment, lymphocytes expressing a predominant TCR V phenotype in tumor tissue or peripheral blood before or after treatment are isolated and expanded by standard procedures.

Antibodies to various TCR Vβ subsets are immobilized on inert solid supports and incubated with blood cells and/or tissue cells to include bone marrow and peripheral blood or lymphoid tissue cells and tumor infiltrating lymphocytes. The bound T cells are eluted with various buffers. Suitable biocompatible inert supports include polystyrene, polyacrylamide, nylon, silica, and charcoal as well as others known in the art. The supports are derivatized for covalent binding of antibodies with agents well known in the art including heterobifunctional compounds, carbodiimide, and glutaraldehyde. The enriched population of Vβ-bearing T cells is then used for in vitro immunization with a SAg in native or mutant form capable of activating the dominant TCR V bearing lymphocytes. IL-2 is used to further expand the cell population as described above.

Effector lymphocytes obtained after in vivo sensitization are stimulated in vitro with tumor associated antigens bound to irradiated PBMC (which act as stimulator cells) for 8-72 hours. DCs, macrophages, or other class I-bearing cells are used to present the tumor associated antigens. The T cells are then analyzed for TCR Vβ and/or CD44 expression. An STCT expressing a SAg is then added to the culture (1 picogram to 10 microgram). If a given V predominance is noted after antigen stimulation, then an STCT or SAg known for its ability to specifically stimulate that Vβ subset is selected for use in activation. Culture proceeds for 18-72 hours. The TCR Vβ and CD44 profile of stimulated T cells are then rechecked. IL-2 (12-25 IU) and/or anti-CTLA-4 antibodies are added for an additional 8-72 hours after which the cells are harvested for use. The optimal timing of STCT introduction after tumor antigen stimulation is between 3 and 14 days.

Antigen-presenting cells (APCs) of all kinds such as DCs, B cells or macrophages with appropriate MHC class II molecule binding sites for soluble SAgs are used or the SAgs are presented alone or in immobilized form without APCs. Optionally, STCTs are used without APCs. Before IL-2 administration, effector cells are re-stimulated weekly by washing and replating in 24 well plates at a concentration of 2.5×10⁵ cells/ml in CM. This is continued for 3-10 cycles until enough cells are available for IL-2 expansion. T cells are cloned 7 days after the several cycles of stimulation in 96-well round bottom plates at 0.3 cells/well with 5×10⁴ stimulator tumor antigen-PBMC, SAg, or STCT and 25-50U recombinant IL-2 in a volume of 200 ml.

For long term growth, clones are transferred to 24 well plates and 1×10⁶ cells/well and stimulated weekly with SAg or STCT plus optimally 5×10⁵ tumor associated antigen-PBMC and 25-50U/ml of IL-2. After clones grow to greater than 2×10⁶ cells, the clones are maintained by culturing with STCT only for 48 hours, washing to remove STCT, and replating in fresh media for 5-7 days with 25-50 U/ml IL-2.

The initial incubation is with the selected tumor associated antigen such as MART-1 for 1-3 days with the latter reagents followed by Vβ profiling and re-stimulation with SAg by methods given above. The MART-1 is presented attached to HLA-A1⁺ cells of PBMC. Cytotoxic activity is tested after the first and/or second rounds of sequential stimulation with tumor associated antigen and SAg given below.

The tumor-specific effector T cell population is immortalized as tumor specific T cell hybridomas. These hybridomas are generated by immunization in vitro of human T cells as described herein. The expanded T cells are then fused to a thymoma and cloned by limiting dilution or other methods well known in the art. Cells are cultured in complete tumor medium composed of Eagle's minimal essential medium supplemented with 10 mM 2-mercaptoethanol, 10% fetal calf serum, 10% Mishell-Dutton Nutrient cocktail, 100 U/ml penicillin G, and 200 mg/ml streptomycin sulfate. Other well known culture media can also be used.

For SAg immunization in vitro, various antigen presenting cells are used including MHC class II-positive T cells as well as those expressing CD1. Purified MHC class II or CD1 molecules alone or immobilized are substituted for APCs in some cases. Moreover, T cells are activated by some SAgs without APCs when presented to T cells in immobilized form or in the presence of various cytokines such as IL-1, IL-2, IL-4, or IL-6 or xenogeneic antigens. Various costimulants such as B7-1 and B7-2, adhesion molecules such as ICAM-1 and VCAM-1, or GalCer are used together with SAgs and MHC class II positive APCs or immobilized MHC class II peptides to augment the T cell or NKT cell response.

Tumor associated antigen immunization is also involved in the binding of peptides to MHC class I bearing APCs of multiple origins. Various cytokines including, but not limited to, IL-1, IL-2, IL-4, IL-12, or LPS are used in vitro or in vivo to expand the antigen specific clone of T cells and avert the development of T cell anergy.

Specialized Forms of Tumor Specific Effector Cells and Hybridomas

Tumor specific T or NKT cells with TCR Vβ and/or CD44 selectivity are produced by transfecting uncommitted stem cells with nucleic acids encoding particular TCR Vβ chains. Likewise, a T cell clone overexpressing CD44 is produced by transfecting T cells with nucleic acids encoding CD44. A hybridoma expressing a tumor associated antigen with a dominant TCR Vβ phenotype or CD44 expression is produced in this way. Such a T cell hybridoma or cell line is stimulated exogenously by a SAg or a SAg mutant with a TCR V or CD44 selectivity corresponding to that expressed predominantly by the T cell hybridoma. The result is a clone of tumor specific T cells capable of being expanded by exposure to SAg in vitro or in vivo.

CD44 expression is induced in a T cell, NKT cell or TCR γ/δ T cell population after activation in vitro or in vivo with SAgs alone or together with any of the T or NKT cell stimulating constructs and methods described herein. The in vivo and in vitro activation steps and immunization protocols are given in Examples 7, 15, 16. 18-23. The CD44 positive T cell population exhibits upregulated primary adhesion properties and is capable of effectively trafficking and homing to tumor cells in vivo and particularly to sites of SAg (in native or nucleic acid form) injection i.e. tumor Nucleic acids encoding CD44 or a carbohydrate modifying agent will induce CD44 expression on the T cell surface. A preferred in vivo method of use involves intratumoral injection of SAg DNA into tumor sites which induces expression of CD44 on T cells resulting in enhanced T cell trafficking to the site of SAg administration.

T cells are genetically engineered to overexpress CD44 after SAg stimulation. This is accomplished by transfection of T cells or NKT cells with nucleic acid encoding CD44 as well as nucleic acids encoding glycosyltransferases. This results in the overexpression of CD44 upregulation of the adhesive properties of CD44. Such CD44 enriched clones are harvested after SAg stimulation, enriched, and administered for adoptive immunotherapy of cancer (Examples 6, 7, 15, 16, 18-23).

Additionally, T or NKT cell clones or hybridomas are produced which express a chimeric TCR consisting of an invariant chain with specificity for GalCer and a chain that binds a SAg. The Vβ region which is specific for the SAg is overexpressed on the TCR permitting greater responsiveness to exogenous SAg. This chimeric TCR recognizes and is stimulated by an exogenous SAg with a TCR Vβ selectivity corresponding to the predominant TCR Vβ phenotype of the T or NKT cell. Such T or NKT cell lines are cloned and hybridomas produced by methods well known in the art.(Current Protocols in Immunology, pp. 7.21.-7.21.9 John Wiley, New York, 1991) The expanded clone of tumor specific T cells produced in this way is useful for adoptive immunotherapy of cancer by methods given in Examples 7. 15, 16, 18-23.

T cell clones are produced due to asynchronous TCR Vβ locus rearrangements at low but significant frequency in which both TCR Vβ segments are part of two functional TCRs. Such clones are produced from uncommitted stem cells in which nucleic acid encoding two chains are transfected, one having specificity for a tumor associated antigen and another having SAg specificity. Hence, a clone of T cells with dual Vβ TCR expression is produced which is capable of reacting with a tumor specific and a SAg. This clone is expanded by binding either or both ligands. These expanded clones consisting of tumor specific effector T cells are used for adoptive immunotherapy of cancer by protocols given in Examples 7, 15, 16, 18-23).

T cells or NKT cells clones or hybridomas expressing TCR Vα and Vβ chains with specificity for GalCer and SAg, respectively, are produced by fusion of NKT cell DNA encoding the GalCer and SAg receptors with DNA from an appropriate thymoma. This GalCer receptor and SAg receptors are expressed on the and chain of the TCR, respectively. Upon exposure to GalCer or SAg, these cells are further activated to express CD44 which enhances their homing and adhesive properties. NKT or T cells expressing high levels of IFN, GM-CSF, and IL-10 are selected and cloned. The clone of T cells producing IFNγ and expressing GalCer, SAg and CD44 is then expanded and immortalized. With its properties of tumor recognition, SAg and glycosylceramide activation, IFNγ production and effective in vivo trafficking, this T or NKT effector cell population is preferred for adoptive immunotherapy of cancer by methods given in Examples 7, 15, 16, 18-23).

Additional measures to avert apoptosis and augment proliferation capacity in SAg activated T cells include the use of anti-CD28 antibodies and inhibition of CTLA-4 on T cells. CTLA-4 on T cells is blocked by specific antibodies or fragments. Alternatively, a T cell clone is used in which CTLA-4 is genetically deleted. When stimulated by SAg, these cells proliferate to a greater extent compared to SAg alone. Cell populations in which CTLA-4 is deleted or blocked are selected to have a predominant V bearing lymphocyte population that is activated after in vivo or ex vivo tumor associated antigen stimulation. After CTLA-4 deletion or blockade, the appropriate SAg with V selectivity is chosen to expand this population. To avert uncontrolled proliferation in vivo, the thymidine kinase gene of the HSV is co-transfected to enable elimination of these cells in vivo if desired.

Measures to produce an effector T cell population with an overexpressed TCR Vβ and/or Vβ chains specific for a given SAg involve the transfection of nucleic acids encoding the desired Vβ or Vβ regions into T cells as in Example 1. To lower the activation threshold of the T cell or NKT cells to SAg or SAg-tumor peptide-MHC or CD1, the T cell or NKT cells are transfected with nucleic acid encoding a tyrosine kinase or other signal transduction initiating molecules which can dimerize in the membrane with the TCR tyrosine kinases thereby lowering the threshold for activating the signal transduction pathway. The deletion of the signal transduction inhibitory region of the TCR to produce sustained signal transduction is done by site directed mutagenesis as in Example 24.

### EXAMPLE 8

Prevention of Anergy in T or NKT Tumor Specific Effector Cells

The SAg stimulated tumor specific effector T cells used for adoptive immunotherapy of cancer may not function when infused unless measures are taken to prevent T cell anergy or activation-induced cell death (AICD) by interdicting the Fas mediated pathway. The Fas ligand (FasL) has been identified as a type II transmembrane polypeptide of the TNF family. These two related receptor-ligand systems signal apoptosis through closely related but distinct pathways. T cell phenotypes that have diminished expression of Fas or FasL show delayed anergy induction and shortened periods of non-reactivity compared to Fas-expressing cells. Activation-induced cell death (AICD) induced by SAgs in vitro or in vivo is averted using Fas-deficient T cells, including those with down-regulated Fas or FasL receptors as well as those with masked or blocked Fas receptors. A Fas-IgG fusion protein is added during the SAg activation phase to prevent AICD or anergy induction. Measures such as those above (or by treating with anti-CTLA-4 antibodies or activation of CD28 before, during, or after STCT stimulation) protect T cells from anergy or AICD. In this way, these manipulations prolong T cell survival in vitro and enhance tumoricidal activity in vivo after the T cells are activated by tumor associated antigen plus SAg or tumor associated antigen-SAg conjugates in vitro.

SAg nucleotide alone or fused to tumor peptide nucleotide may be further fused with an antisense nucleotide capable of inhibiting the apoptosis pathway.

When expressed in T cells, this combination of genes would promote the generation of tumor specific effector T cells which would be resistant to AICD. Oligonucleotide antisense molecules that inhibit key steps leading to apoptosis may be fused to SAg DNA in order to prevent the T cells from undergoing AICD. SAg DNA may also be fused with the multi-drug resistance (MDR) gene to make the T cells refractory to chemotherapeutic agents and sensitive to anti-apoptosis drugs. Certain drugs or radiation may be used together with SAg DNA for additive or synergistic inhibition of the apoptosis pathway in the doubly or multiply transfected T cells.

SAg DNA may also be linked operatively to promoter genes such as those inducible by corticosteroids or heavy metals (e.g., the metallothionein promoter) and regulatory DNA sequences that act as T cell on/off sensors responsive to exogenous cytokines, inflammatory stimuli and changing external conditions such as oxygen tension and pH. A particular advantage of SAg DNA is that its expression will promote Vβ receptor downregulation and internalization so that these receptors are unavailable to exogenous SAg. SAg DNA is modified in several ways to introduce protein binding sites for key transcriptional elements which may inhibit apoptosis. Insertion of such sites at the bending domains of SAg oligonucleotides renders them capable of inducing key TH-1 cytokines and cell proliferation while averting AICD. SAg DNA is also capable of reversing the T cell anergy and signaling defect which may be localized to the chain in cancer patients. This is accomplished by providing transcriptional binding sites on the SAg DNA which bypass the conventional chain activating signals and the pathway to IFNγ and IL-2 production. In the same way, SAg DNA also bypasses the defective signal by activating a complex that contains STAT-1 which binds a GAS-like palindromic sequence located in the IFNγ response region of the FcRI gene. Such anergy in T cells may also be reversed by alternate cytoplasmic tails that are activated by SAg binding to the TCR Vβ and Vβ chains. Moreover, nucleic acid encoding Protein A and especially domain D (that binds to the Ig VH3 region) may be fused to SAg DNA in order to bring about activation of the IL-2 and IFNγ genes that resulting in T cell proliferation and IFN production coupled with up-regulated surface receptors for the Ig VH3 domain.

Anergy in SAg-activated tumor-specific T or NKT effector cells (or hybridomas) is known to be averted by in vitro or in vivo co-administration of IL-2, IL-1, LPS and tumor specific peptides specifically interfere with SAg driven anergy.

Methods and doses for use of these agents with SAg activated T or NKT cells are given in Examples 7, 15, 16, 18-23. Tumor specific T or NKT effector cells or hybridomas prepared by various methods described above are administered according to the adoptive therapy protocol of Examples 7, 15, 16, 18-23 (the preferred method). The experimental tumor models and human cancers for which the anti-cancer efficacy of these cells can be demonstrated are provided in Example 16.

### EXAMPLE 9

Reactivation of Anergized Tumor-Specific T or NKT Cells by SAg and SAg Receptors

Preferred tumor-specific effector cells for adoptive immunotherapy of cancer are autologous T cells. However, in the course of tumor growth, T cells become anergized to the host's own tumor and are incapable of an adequate immune response to the tumor. Dampened TCR-triggered responses are caused by suppression of effector molecules that couple cell surface receptors to early and late intracellular signaling events. For example, basal and induced tyrosine phosphorylation of many signaling proteins is reduced due to deficits at multiple points, including the inositol phosphatase pathway. This down-regulates cytokine production and decreases nuclear transcription factors of TH1 helper cells.

Two functionally distinct signal transduction pathways are coupled to the TCR. Native or mutant SAgs activate anergic T cells via an alternate pathway without the conventional increases in Ca++ mobilization or detectable phosphatidylinositol hydrolysis that follow ligation of the TCR by peptide/MHC complexes. Native, mutant or derivatized SAgs are administered to stimulate anergized T and/or NKT cells to become tumor-specific effector cells now fully reactive against tumors. Such cells are used also in adoptive immunotherapy of cancer as described in Examples 7, 15, 16, 18-23. Nucleic acid constructs comprising DNA encoding SAg and SAg receptor are provided to reverse T cell anergy in cancer patients.

Anergic T (or NKT) cells transfected with DNA encoding a SAg receptor express the receptor on the cell surface. Binding of exogenous SAg to this receptor generates T cell activating signals, so that the activated T cells can be used for adoptive immunotherapy.

DNA encoding a SAg peptide is transfected into cancer patients' anergized T and/or NKT cells. These DNA constructs also contain the ISS (described above). The T and/or NKT cell transfectants have revitalized proliferative activity when stimulated by tumor-specific antigen and exogenous SAg. The cells are used for adoptive immunotherapy of cancer (Examples 7, 15, 16, 18-23).

Anergic T cells from cancer patients are transfected in vivo or in vitro, resulting in a population of tumor reactive effector T cells in vivo or ex vivo. The ex vivo transfected T cells are used for adoptive immune therapy as described in Example 15, 16, 18-23. In the case where SAg receptor is expressed by transfected T cells, these cells are activated by locally or systemically by SAgs to result in tumor-specific effector cells.

Additional manipulations that assist in restoring responsiveness to anergic T cells include removal of the (T or NKT) cells from the immunosuppressive microenvironment and transfer into tissue culture for a short period before stimulation with SAg. Furthermore, defective signaling in patient T or NKT cells may be reconstituted by transfection with DNA encoding CD3-2 or fyn that is either in a single construct with, or cotransfected with, DNA encoding SAg and/or SAg receptor. Now, surface activation of the SAg receptor triggers CD3-signaling and T cell proliferation.

SAg activation of a T cell surface ganglioside (such as GD3) is also used to reverse T cell anergy in cancer patients. SAg coupled to GalCer, lipopolysaccharides or proteosomes are even more effective in activating such anergized T cells. Coordinate activation of CD69 with phorbol esters in combination with SAg stimulation also reverses T cell anergy.

### EXAMPLE 10

Tumor Specific Effector T or NKT Cells as Lymphoid Organoids

Tumor specific T and/or NKT effector cells (or hybridomas with such cells) are prepared ex vivo in the form of a lymphoid organoid and implanted into tumor-bearing hosts. The organoid consists of the tumor specific lymphocytes either activated by SAgs, transfected to express SAg alone or in combination with the other proteins or anti-tumor moieties described herein. The cells are encased in semi-permeable membranes that allow for their progressive entry into the blood and lymphatics after implantation into the host. Such organoids are implanted preferentially at sites adjacent to lymphatics or blood vessels that drain organs or regions of known tumor involvement. However, they may also be implanted subcutaneously, intraperitoneally in addition to intra-tumorally or adjacent to a tumor site. The advantage of the organoid is that it continuously provides proliferating tumor-specific effector cells that recognize traffic to tumor sites in a physiological manner. This approach avoids negative selection, functional deficiencies and storage problems associated with long term cultured cells.

Organoids are encased in macrocapsules, sheaths, rods, discs, or spherical dispersions or microcapsules. Microcapsules are made of hydrogels such as polysaccharide alginate that are optionally coated with polyanions and again with alginate. Macrocapsule and vascular devices consists of acrylonitrile-vinyl chloride copolymers or cellulose nitrate membranes. In one approach, scaffolds composed of synthetic polymers serve as cell transplant devices. The polymers are degradable or non-degradable materials that disappear from the body after they perform their function to obviate concerns about long-term biocompatibility.

These devices serve as structural and functional tissue units by the transplanted cells. The open system implants are designed so that the polymer scaffold guides cell organization and growth and allows diffusion of nutrients and cells. The cell polymer matrix is pre-vascularized or becomes vascularized as the cell mass expands after implantation. Vascularization is induced naturally by the host or artificially by secretion of angiogenic factors from host cells. Optionally, the angiogenic proteins are genetically engineered into the host T cells in vitro before implantation or in vivo before or after implantation.

To maintain or facilitate targeting of the cells to tumors or involved organs, the lymphocytes are transfected with DNA encoding polypeptides that enhance homing and trafficking ability to the sites of tumor burden (e.g., brain, liver, lung). The organoid lymphocytes express no CTLA so that they may proliferate (in vitro and in vivo) without the need for exogenous IL2. Alternatively, cells are transformed to express herpes simplex virus thymidine kinase, making them susceptible to killing by gancyclovir. This curtails uncontrolled proliferation caused by the CTLA-4 deletion (or inhibition). Exogenous control of antitumor activity is achieved through the use of inducible promoters, such as those responsive corticosteroids or metals.

### EXAMPLE 11

Tumor Specific Effector Cells or Tumor Cells Expressing Protein A, Protein A Domains and/or Angiostatin

It is desirable to express Fe receptors (FcR) or Ig VH3 domains on tumor cells to promote binding by immunoglobulins and enhance damage by antibody dependent cellular cytotoxicity. By introducing Staphylococcal Protein A, or its domains A-D into tumor cells which overexpress FcR and VH3 the tumor cells bind immunoglobulins (including those withaGal specificity). Signaling of T cells occurs via high affinity binding to FcR (FcRI) of protein A-IgG complexes; such binding bypasses the CD3-blockade in tumor bearing patients. These transfected tumor cells are useful as a vaccine. Likewise, nucleic acids encoding protein A and its domains A-D are transfected into partially or fully anergized T or NKT cells of cancer patients. Exogenous immunoglobulins stimulate the generation of tumor-specific effector T or NKT cells which are used in adoptive immunotherapy (Examples 7, 15, 16, 18-23).

DNA encoding Staphylococcal protein A and its domain D are co-transfected into these tumor cells resulting in the joint surface expression of: (1) protein A and FcR to which it binds and/or (2) domain D and Ig VH3 to which it binds.

When DNA encoding protein A or domain D, fused to a signal sequences that route and anchor the protein A peptide to the tumor cell surface, is introduced into tumor cells, such tumor cells are excellent targets for parenterally administered SAg polypeptides (particularly those for which no natural antibodies exist). Tumor cells expressing protein A and domain D and also expressing FcRs on the cell surface, have heightened sensitivity to complement mediated lysis.

Tumor cells cotransfected to express protein A and Gal (by introduction of the appropriate glycosyltransferase) are capable of reacting with natural anti-Gal antibodies, Ig Fc fragments and Ig VH3 domains, which stimulate an enhanced tumoricidal response.

Angiostatin is a circulating angiogenesis inhibitor which is 38-kDa internal fragment of (mouse) plasminogen that contains the first four disulfide-linked kringle domains. In vivo, angiostatin suppresses neovascularization in several traditional assays (chick chorioallantoic membrane assay and mouse corneal assay). Proteases released by tumor cells cleave circulating plasminogen to generate angiostatin. Metalloelastase produced by tumor infiltrating macrophages generated angiostatin production by murine Lewis lung carcinoma. In the present invention, nucleic acid encoding angiostatin (Cao Y et al., J. Clin. Invest. 101: 1055-1063, (1998)) are cotransfected into tumor cells with nucleic acid encoding SAg (as in Example 1). The tumor cell cotransfectants express and secrete SAg and angiostatin. Such cells are used directly as a preventative vaccine (Example 8) or as a therapeutic vaccine to treat established tumor including micrometastases. Methods for using these cells in vivo are in Examples 7, 12, 16, 18-23.

In addition, tumor cells are cotransfected to express angiostatin and protein A (and/or its domains). Any nucleic acid construct shown in Table I may also be used in combination to transfect tumor cells together with protein A, its domains and angiostatin.

### EXAMPLE 12

SAg Receptor

Colon carcinoma is used as the tissue source for the SEB receptor. Mixtures of different detergents at low concentrations are used. The protocol for screening detergents for solubilization of MAChRs is readily adaptable to other receptor types. The membranes are suspended at 5-10 pH 7.5, 20 mM Tris-HCl, pH 7.5, or 20 mM sodium phosphate, pH 7.0-7.5. For screening purposes it is unnecessary to add complex proteolysis inhibitor cocktails. The presence of EDTA (1 mM) to inhibit calcium-activated proteases and of PMSF or benzamidine (0.1 mM) to inhibit serine proteases is sufficient. Mg2⁺⁺ (2 mM) is added. The membranes are prelabelled with a radioligand in the presence and absence of a suitable unlabelled ligand to determine the total and non-specific binding. Non-specific binding is subtracted from total binding to obtain the specific binding. A high enough concentration of labeled ligand to saturate the binding site(10×Kd) is used, so that the binding capacity is measured. The unlabelled ligand is used at a concentration of 1000×Kd. The normal criteria for specific binding must be fulfilled. The incubation is sufficient to reach equilibrium. Prelabelled membrane suspension (0.5 ml) is added to a series of centrifuge tubes a 4° C. An equal volume of detergent solution in the same buffer is added to obtain a series of different final detergent concentrations, e.g., 0, 0.1, 0.2, 0.5, 1.0, 2.0% w/v. The tubes are mixed and incubated for 60 min. at 4° C. Solubilization is assisted by stirring or mixing, e.g., with a rotating-wheel end-over-end mixer. The tubes are centrifuged for 30-60 min at 100,000×g for 60 min. For screening, a lower speed spin, e.g., 10,000×g for 5 min (such as in a microfuge) is acceptable. Supernatant, 0.2 ml, is applied to a 2 ml column of Sephadex G50 equilibrated with the selected detergent at 0.1%. When the sample has run in, 2×0.2 ml of detergent-buffer is applied and then the void volume fraction is eluted with 0.5 ml of detergent buffer. This procedure is carried out, the remaining material is removed, and 10 ml of aqueous scintillation cocktail is added and the radioactivity counted. Sephadex G50 is substituted for G50F for hydrophilic ligands, which do not partition into detergent micelles. This gives a more rapid separation. The recovery of specifically bound ligand is calculated in absolute terms:

bound ligand=(dpm(total)−dpm(non-spec.)×5/(2220×spec. .act) pmol/ml

An aliquot of the pellets is resuspended and counted to calculate recovery of unsolubilized receptors. The concentration of protein in the solubilized supernatant is measured, for example, by measuring UV absorbance at 280 nm against a detergent-buffer blank. (If necessary, the supernatant is diluted to get the absorbance on scale.) Protein concentration in the solution is approximately equal to the absorbance at 280 nm. Alternatively, the Lowry method is used. The above steps are repeated without first prelabeling the receptors in the membrane. Instead, the solubilized supernatant is incubated in the absence and presence of labeled ligand. Again, concentration of the labeled ligand is used that saturates the binding sites. Incubation is carried out for 2 h at 4° C., and the binding is assayed by gel filtration as above. The pellet is resuspended and assayed for residual binding to check overall recovery. The molecular size of the receptors in solubilized preparations is estimated by a combination of gel filtration chromatography and sucrose density gradient centrifugation in H₂O and D₂O. Affinity chromatography is the principal method use for purification of all of the receptors, combined with gel permeation HPLC, and ion exchange. SDS PAGE is carried out on the final product. Affinity chromatography is carried out using immobilized SEB, and the column is eluted with acid buffer or different concentrations and ionic strengths of eluting buffer.

Determination of Amino-Acid and Oligonucleotide Sequences of SAg Receptors

Receptor material is eluted from the SDS-PAGE, and the N-terminal amino acid sequence is determined. When free amino termini are not available, the purified receptor material must be subjected to partial hydrolysis. The specific cleavage of peptide bonds is performed with endoproteases, such as V8 protease or trypsin, or with chemicals such as cyanogen bromide(CNBR). The resulting peptides are separated by SDS-PAGE when they are over residues or by reverse phase HPLC. The peptides thus analyzed are subjected to amino-acid sequence analysis with a gas phase or solid phase sequencer.

Antibodies are raised against the peptides. and the resultant antibodies used to confirm that the peptide is a part of the receptor by immunoprecipitation or Western blot.

To determine the full sequence of the receptor gene, oligodeoxynucleotide probes synthesized on the basis of peptide sequences are used to screen an appropriate cDNA library. Either a mixture of relatively short oligonucleotides with all possible sequences or a relatively short oligonucleotides with a sequence based on codon usage frequency is used. Genomic libraries as well as cDNA libraries are screened to obtain genes for receptors and to deduce their amino acid sequence. The amino acid sequence deduced from the nucleotide sequence is compared to the known sequences of other receptors. Among the useful structural information derived from the sequence analysis is the hydropathy profile. The presence of hydrophobic domains with a length of approximately 20 amino acids residues suggests that the regions are transmembrane segments. Genomic or cDNA clones ligated into expression vectors are used to transform suitable cell lines.

Alternatively, mRNA transcribed from these clones is injected into recipient cells such as Xenopus oocytes. The expression of receptors in these cells is confirmed by measuring ligand binding, reactivity of cell homogenates or membrane preparations with antibodies or the responses induced by receptor agonists in recipient cells. The direct function of the receptors is elucidated by reconstituting purified receptors in phospholipid vesicles with or without other components.

An additional method is based on the isolation of cDNA or genomic clones for receptors without using purified receptors. The structure of receptors and cellular responses to them is examined using these clones. Substantial amounts of receptor material is produced from these clones. Monoclonal antibodies to the SAg receptors are used to screen clones for receptors derived from cDNA libraries constructed with expression vectors.

Transfection of SAg receptor involves the ligation of the receptor gene into an appropriate expression vector, transformation of a suitable bacterial host, and isolation of an individual bacterial colony containing the plasmid vector. The plasmid DNA is harvested from the lysed bacteria. The preferred method of purification of plasmid DNA for use in transfections involves Triton-lysozyme equilibrium gradient. The cells to be used in transfection are maintained in the log phase of growth at all times. The calcium phosphate method is useful and efficient means for introduction of cloned genes in plasmid vectors into mammalian cells as described earlier in this document is preferred. However, the other methods given are useful as well. A partial list of plasmid vectors and promoters suitable for transfection of cultured mammalian cell is given in Fraser, C. M., Expression of Receptor Genes in Cultured Cells in Receptor Biochemistry, A Practical Approach, Hulme, E. C., ed., Oxford University Press, pp. 263-275, 1993.

### EXAMPLE 13

Avoiding Interference with SAg-Specific Antibodies

Naturally antibodies are found in mammals that are specific for the SAg molecule (e.g., a Staph enterotoxin). Such antibodies bind and interfere with the SAg expressed and secreted by transfected cells. Such antibodies also hinder therapeutic action of SAg infused directly (as native protein, peptide or fusion protein).

It is desirable to neutralize or otherwise remove such before the cells of this invention are administered to a subject. One way to achieve this is to pre-treat the subject with antiidiotypic antibodies specific for the variable region of SAg-specific antibodies. Another way is to infuse SAg peptides that represent the major immunogenic portions of the overall protein. Alternatively, SAg is immobilized to a solid support by covalent bonding and the blood or plasma is perfused extracorporeally through a device containing the immobilized protein, thereby removing the antibodies by immunoadsorption. In another approach, SAg-expressing cells (prokaryotic or eukaryotic) preferably of host origin, or phage displays, are encapsulated and used as immunoadsorbents to binds circulating SAg-specific antibodies. An organoid containing these adsorbing cells is positioned subcutaneously or placed into the circulation via catheter and then removed once the adsorption process is complete. Alginate encapsulated cells expressing SAg are preferred but other known modes of cell encapsulation may be used. Liposomes with surface-bound SAg are another form of immunoabsorbent that are employed either as an organoid or by direct injection.

Induction of Immunological Tolerance

The induction of tolerance to epitopes of the SAg molecule which induce a humoral antibody response would be desirable. The portion of the SEA molecule which binds to natural antibodies is the linear sequence of residues 232-262. Immune tolerance is induced using this sequence by the method of Dintzis et al., Proc. Natl. Acad. Sci. 89: 1113-1117 (1992). in which low molecular weight peptide arrays are administered to patients with circulating antibodies to enterotoxins. The peptides are delivered a parenterally or orally once weekly in doses of 1-500 mg/kg for three to six weeks after which there is a reduction and disappearance of circulating antibody specific for the tolerogen.

After one or more of the foregoing treatments, native SAg or SAg conjugated to a monoclonal tumor-specific antibody and administered to the host can now localize to tumor sites without diversion by circulating SAg-specific antibodies.

Phage Displayed SAgs

Phage display technology may also be used neutralize circulating anti-enterotoxin antibodies. The SAg and/or SAg receptor is expressed at the surface of bacteriophage as a fusion protein with the gene VIII protein (gVIIIp). This phage-displayed SAg fusion protein retains the properties of the natural protein. For this invention, the filamentous phage vector f88-4 which forms a fusion protein between the C terminus of the inserted gene product and the N terminus of gVIIIp is used. The phage expressing SEA is injected intravenously into patients that have natural antibodies to SEA. The amount of phage (transducing units) required to neutralize the circulating pool of antibodies is predetermined by antigen binding inhibition assay. The number of transducing units required to neutralize the pool of circulating SEA specific antibodies is administered intravenously. Shortly after this injection, the host is ready for treatment with active SEA which is no longer hindered from finding its “target,” i.e., enterotoxin receptors expressed by tumor cells or T cells.

SEA clone pKH-X35 is employed. PCR with Vent Polymerase 9NEB is used to mutate the 5′- and 3′-ends of the SEA gene for cloning into f88-4. The construct is as follows. The 5′ oligonucleotide used is (SEQ ID NO: 145) 5′-CTCCAAGCTTTGVCCAGCGAGAAAAGCGAAG-3′. Two 3′ oligonucleotide primers are used. For the construct with the five amino acid linker between SEA and gVIIIp (SEA L), the primer (SEQ ID NO: 146) 5′-GCCTCCTGCAGATCCACCGCCTCCGGATGT-ATATAAATATATATC-3′ and for the non-linker version (SEA-P); (SEQ ID NO: 147) 5′-GCCTCCTGCAGATGTATATAAATATATATC-3′ are used. The two SEA PCR products are cut with HindIII and PstI and cloned into f88-4. They are transformed by electroporation into E. coli strain DH5a and sequenced. Phage are produced by growing the transformed bacteria overnight in 0.5L of broth with 20 mg/ml tetracycline. The culture is pelleted twice (800×g for 15 min) and the phage precipitated out of the cleared supernatant by the addition of 0.15 vols. of PEG/NaCl solution (17% PEG 8000, 19% NaCl in water). After incubation at 4° C. for 2 hours, the phage are resuspended in TBS and sterile-filtered through a 0.22-m membrane. Phage are selected by the micropanning technique and by cell binding. Binding to antibody is assessed by attaching mAb to the surface of 96-well ELISA plates, blocking with 1% BSA, incubating with 100 mg/ml of SEA or PBS as a control and then incubating with the various phage preparations for >2 hours at 4° C. The phage is then eluted with 0.1 M HCl pH 2 ( adjusted with glycine) for 10 minutes, neutralized and used to infect starved E. coli MC10161 F′ Kan. The infected bacteria are then spread on tetracycline (20 mg/ml) LB agar plates. After overnight culture tetracycline resistant colonies are counted representing the number of transducing units (TU) recovered. To determine the number of SEA-bearing phage among the tetracycline-resistant colonies, colony blotting is performed by standard techniques probing with a ³²P-labeled SEA probe. An antibody based variant of this technique is involves probing with a rabbit anti-SEA serum as for Western blots.

Chimeric Enterotoxins

Likewise, hybrid or chimeric SAgs that are non-immunogenic are used to stimulate cells. When these molecules are injected into hosts that have natural antibodies, they are not rapidly eliminated from the circulation. Such chimeric molecules lacking the binding site for natural antibodies preserve the T cell mitogenic and cytokine-inducing properties of the native SAg. A peptide sequence from another SAg to which antibodies do not exist is substituted using genetic or biochemical methods well known in the art. This is particularly useful in the case of enterotoxins such as SEB or SEA to which a large percentage of humans have naturally occurring circulating antibodies. The antibody binding region of these molecules near the C terminal regions is delineated. The substitution of the antibody binding sequences in SEA or SEB for sequences from SEE or SED to which a very small number of humans have circulating antibodies markedly enhances the tumor killing efficacy of the injected chimeric enterotoxins. A hybrid molecule consisting of a 26 amino acid peptide corresponding to the N-terminal portion of SEA, the loop structure of SEA, a conserved mid-molecular sequence of SEA and SEB, and a C terminal sequence of SEB was synthesized in collaboration with Multi-Peptide Systems, La Jolla, Calif. Peptides were prepared using a variation of Merrifield's original solid phase procedure in conjunction with simultaneous multiple peptide synthesis using t-Boc chemistries. Peptides were cleaved from the resins using simultaneous liquid HF cleavage. The cleared peptides were then extracted with acetic acid and ethyl ether and lyophilized. Reverse phase HPLC analysis and mass spectral analysis revealed a single major peak with the molecular weight corresponding closely to theoretical.

Synthetic SAgs

Amino acid sequences of SEA and SEB known to be involved in the interaction with the TCR and MHC class II molecules are retained. The loop structure of SEA is retained because it is devoid of histidine moieties that are associated with the emetic response. Residues 1-10 of the N-terminal region of SEA are retained because they have MHC class II binding activity. The loop structure of SEA is retained because it and associated disulfide linkages are considered to be important for T lymphocyte mitogenicity, stabilization of the molecule, and resistance to in vivo degradation. A conserved sequence in the central portion of SEA and SEB adjacent to the disulfide loop (amino acids 107-114) was retained. Histidine moieties are deleted from the molecule because of their association with the emetic response.

Synthesis Procedure

The preparation of all peptides was carried out using a variation of Merrifield's original solid phase procedures in conjunction with the method of Simultaneous Multiple Peptide Synthesis using t-Boc chemistries (Merrifield RBI, J. Amer. Chem. Soc. 85:2149-2154 (1963)); Houghten R A, Proc. Natl. Acad. Sci. USA 82:5131-5135 (1985); and Houghten R A et al., Intact. J. Peptide Protein Res. 27:673-678 (1985)).

4-methylbenzhydrylamine (mBHA) and phenylacetamidomethyl (PAM) resins were purchased from Advanced Chemtech (Louisville, Ky.) and Bachem (Torrance, Calif.), respectively. All of the amino acids contained the t-butyloxycarbonyl (t-Boc)-amino protecting group and were purchased from Bachem. The side chain protecting groups included benzyl (threonine, serine and glutamic acid), chlorobenzyloxycarbonyl (lysine), bromobenzyloxycarbonyl (tyrosine), cyclohexyl (aspartic acid), p-toluene sulfonyl (arginine), formyl (tryptophan), methyl benzyl (cysteine), and dinitrophenyl or benzyloxycarbonyl (histidine). Cysteine with the HF stable acetamidomethyl (ACM) protecting group was used, upon request, for internal cysteines. Each lot of amino acid derivative was tested by melting point analysis. Reagent grade methylene chloride (CH2Cl2), isopropanol (IPA), and dimethylformamide (DMF) were obtained from Fisher Scientific (Tustin, Calif.). diisopropylcarbodiimide (DIPCDI) and diisopropylethylamine (DIEA) were purchased from Chem Impex (Wood Dale, Ill.). Trifluoroacetic acid was purchased from Halocarbon (Hackensack, N.J.).

The appropriate resin, mBHA for C-terminal amides and PAM for C-terminal acids, was weighed with a Mettler AE 240 balance (Highstown, N.J.) into separate polypropylene mesh (74 mm) packets which had been pre-sealed on 3 of 4 sides using a TSW TISH-300 Impulse Sealer (San Diego Bag and Supply; San Diego, Calif.). Each packet was also pre-labeled with a reference code using a KOH I NOOR Rapidograph pen with graphite based ink to allow them to be easily identified during resin addition and during the synthesis process. Each packet was then carefully sealed completely to make sure there would be no resin leakage. All the resin containing packets (up to 150) were then placed in a common Nalgene bottle. Enough CH2Cl2 to cover all the packets was then added to the bottle, which was then capped and vigorously shaken for 30 seconds on an Eberbach Shaker (Fisher Scientific; Tustin, Calif.) to wash and swell the resin. The CH2Cl2 solution was then removed. All subsequent steps involved the addition of enough solvent to cover all the packets and vigorous shaking to ensure adequate solvent transfer. The N—t-Boc was removed by acidolysis using a solution of 55% TFA in CH2Cl2 for 30 minutes, leaving the TFA salt of the a-amino group. The TFA wash solution was then removed. The packets were then washed for 1 min with CH2Cl2 (2×), IPA (2×) and CH2Cl2 (2×) to squeeze out excess TFA and to prepare for neutralization. The TFA salt was neutralized by washing the packets three times with 5% DIEA in CH2Cl2 for two minutes each. This was followed by two washes with CH2Cl2 to remove excess base.

The resin packets were then removed from the common Nalgene bottle and sorted according to computer generated checklists in preparation for coupling. This was double checked to ensure the packets were added to the correct amino acid solution. The packets were then added to bottles containing the appropriate 0.2 M amino acid in CH2Cl2 and/or DMF depending on solubility. These solutions were also prepared using computer generated information. An equal volume of 0.2 M DIPCDI was then added to activate the coupling reaction. The bottles were then shaken for one hour to ensure complete coupling. At completion, the reaction solution was discarded and the packets were washed with DMF for 1 min to remove excess amino acid and the by-product, diisopropylurea. A final CH2Cl2 wash as then used to remove DMF. The packets were then removed from their individual coupling bottles and placed back into the common Nalgene bottle.

The peptides were then completed by repeating the same procedure while substituting for the appropriate amino acid at the coupling juncture. The packets were then taken through a final acidolysis along with subsequent CH2Cl2, IPA and CH2Cl2 washes to leave the peptides in the TFA salt form. The packets were then dried in preparation for the next process.

Final side chain deprotection and cleavage of the anchored peptide from the resin was achieved through simultaneous liquid HF cleavage (Houghten R A et al., supra. Gaseous N2, HF, and argon were acquired from Air Products (San Diego, Calif.). Anisole was purchased from Aldrich Chemical Co. (Milwaukee, Wis.). Acetic acid (HOAc) and ethyl ether were purchased from Fisher Scientific (Tustin, Calif.). Each packet along with a Teflon coated stir bar was placed into an individual reaction vessel of a multi-vessel hydrogen fluoride apparatus (Multiple Peptide Systems; San Diego, Calif.). An amount of anisole equaling 7.5% of the expected volume of HF was then added to act as a carbonium ion scavenger. The reaction tubes were lubricated with vacuum grease at the point where each contacts the apparatus and sealed onto the HF system. The system was then purged with N2 while cooling the reaction vessels to −70° C. using an acetone/dry ice bath. HF (g) was condensed to the desired level and temperature elevated to −10° C. using ice and water. The reaction was allowed to proceed for 90 minutes with the temperature slowly rising from −10° C. to 0° C. HF was removed using a strong flow of N2 for 90 minutes followed by the use of aspirator vacuum for 60 minutes while maintaining the temperature at 0° C. The reaction vessels were then removed from the apparatus and capped. The residual anisole was removed with two ethyl ether washes. The peptide was then extracted with two 10% HOAc washes. A 50 ml sample of the crude peptide was taken and run on an analytical Beckman 338 Gradient HPLC System (Palo Alto, Calif.) using a Vydac C18 column to profile the initial purity of the compound. The crude peptide was then lyophilized twice on a Virtis Freezemobile 24 Lyophilizer, weighed and stored under argon.

Analytical RP-HPLC was used to determine the homogeneity and approximate elution conditions of the peptides produced. HPLC grade acetonitrile (ACN) was purchased from Fisher Scientific (Tustin, Calif.). HPLC grade TFA was obtained from Pierce Chemicals (Rockford, Ill.). RP-HPLC analysis was carried out on a Beckman 338 Gradient HPLC system (Palo Alto, Calif.) equipped with a BioRad AS-100 autosampler and a Shimadzu CR4A integrator. The column used for all analyses this quarter was a Vydac C-18 column (4.6×250 mm). The solvent system used was 0.05% aqueous TFA (A) and 0.05% TFA in ACN (B) with a flow rate of 1 ml/min. Absorbance was measured at 215 nm. Most peptides were analyzed using the following special gradient; 5.60% (B) in 28 minutes. Hydrophobic peptides were analyzed using the following special gradient: 5-40% (B) in 9 minutes, 40-90% (B) for 10 additional minutes, 95% (B) for the last 9 minutes.

Analytical data was reviewed. The product peak was identified and marked based upon knowledge of common impurities and the use of predicted HPLC retention times.

Peptides that did not meet normal purity requirements for crude material were purified using preparative RP-HPLC techniques. HPLC grade acetonitrile (CAN) was purchased from Fisher Scientific (Tustin, Calif.). HPLC grade TFA was obtained from Pierce Chemicals (Rockford, Ill.). Purification was carried out on a Waters Delta Prep 3000 with a Preparative Waters Prep Pak Module Radial Compression C18 column (5 cm×25 cm, 10-20 m). The solvent system used was 0.05% aqueous TFA (A) and 0.05% TFA in ACN (B). The crude peptides were solubilized in an HOAc/H20 mixture and injected onto the column with 0.25% to 0.50% ACN per minute linear gradient. The absorbance was measured at 230 nm and 40 ml fractions were collected upon elution with an ISO Fraction Collector (Lincoln, Nebr.). The preparative profile was reviewed and selected fractions were analyzed by analytical RP-HPLC. The analytical data was reviewed and fractions were combined and lyophilized. The lyophilized material was weighed, sampled for a final analytical RP-HPLC analysis and stored under argon in powder form. This process was repeated if the purity level attained was not sufficient. Mass spectral analysis was used to determine the molecular weight of the peptides produced. 95% ethanol was purchased from Fisher Scientific (Tustin, Calif.). HPLC grade TFA was obtained from Pierce Chemicals (Rockford, Ill.). Nitrocellulose matrices (targets) were purchased from Applied Biosystems (Foster City, Calif.).

The samples were solubilized in a 1:1 solution of 95% ethanol and 0.1% TFA (aqueous). The samples were applied to a nitrocellulose matrix (Target). The mass spectra were obtained using an ABI Bio-Ion 20 Mass Spectrometer (Foster City, Calif.). The apparatus makes use of plasma desorption ionization via a Cf252 source. The ionized molecules are then analyzed via time-of flight. An accelerating voltage of 15,000 V is used to accelerate the particles.

The Protocol for Intramolecular Disulfide Bridge:

Dissolve crude peptide (300-500 mg) in 200 ml of deoxygenated water and adjust the pH to 8.5 using NH4OH 28%=Solution A. Note: If the peptide is not very soluble in water, some MeOH can be added.

Dissolve 0.5 g K3Fe(CN)6 in 200 ml of deoxygenated water and adjust the pH to 8.5 using NH4OH 28%=Solution B. Note: 0.5 g K3Fe(CN)6 is an average value for 500 mg of a 10 mer peptide. The excess of K3Fe(CN)6 should be approximately 3×. It can be adjusted.

Solution A is then dropped slowly into solution B over a 2 hour period. The mixture is then allowed to react, for an additional 1 hour with stirring. The pH is then adjusted to 4.0-4.5 with 10% ACTH. This solution is injected directly into a preparative RP-HPLC. The major peak is then collected. This “pseudo dilution” technique favors the intramolecular disulfide. Therefore, the major peak is the cyclic product.

The chimeric enterotoxin molecule was tested in normal rabbits and rabbits with established VX2 carcinoma. It was administered intravenously and peripherally with adjuvant. The chimeric molecule (1 mg/ml) was diluted initially in 1 ml of sterile H2O. When the solution was clear, 9 ml of normal saline was added. The solution was filtered through a 0.45 m filter and stored in 0.5-1 ml aliquots. Dosage ranged from 2.6-5.0 mg/kg and was described over 3 minutes via the lateral ear vein in a volume of 0.05 ml diluted further in 1.0 ml of 0.15 M NaCl:

The i.v.. line was then washed with 3 ml of 0.15M NaCl.

In two animals, the temperature rose only 0.3F over the ensuing 24 hours and there was no discernible toxicity over the ensuing 14 days of observation. One animal was described a second dose of the chimeric molecule in pluronic acid triblock adjuvant. This was described in a dose of 8.5 mg subcutaneously in each thigh with a total dose 5 mg/kg. The pluronic acid triblock preparation was prepared as follows: 4.23 cc PBS; 0.017 cc Tween; 0.05 cc Squalene; and 0.25 cc Pluronic. The PBS and Tween were mixed first then squalene was added followed by pluronic acid. The total mixture was vortexed for 3-4 minutes. Two ml of above plus 0.34 ml of the chimeric protein (34 mg) plus 1.66 cc PBS were added to the mixture. The mixture was vortexed vigorously for 1-2 minutes. One ml was injected into each thigh (total vol. injected was 0.17 ml or 17 mg protein or 5 mg/kg).

For nearly 5 weeks after injection, no adverse effects were noted. The tumor showed slow, but progressive growth over this period of time. To date, the chimeric enterotoxin molecule appears to be safe in animals and no untoward side effects were demonstrated. The adjuvant used for these studies was the pluronic acid triblock copolymer which has been used to boost the immune response to various antigens in animal models and which is under testing at this point in humans with hepatitis and herpes simplex infections. Other adjuvants including those prepared in water and oil emulsion and aluminum hydroxide to administer various SAgs in vivo to tumor bearing rabbits were also used.

Additionally, enterotoxins such as SEE, SED, SEC, and TSST-1 are used to prepare hybrid molecules containing amino acid sequences and homologous to the enterotoxin family of molecules. To this extent, mammary tumor virus sequences, heat shock proteins, stress peptides, Mycoplasma and mycobacterial antigens, and minor lymphocyte stimulating loci bearing tumoricidal structural homology to the enterotoxin family are useful as anti-tumor agents. Hybrid enterotoxins and other sequences homologous to the native enterotoxins are immobilized or polymerized genetically or biochemically to produce the repeating units and stoichiometry required for (a) binding of accessory cells to T lymphocytes and (b) activation of T lymphocytes.

### EXAMPLE 14

Pharmaceutical Compositions and Their Manufacture

The pharmaceutical compositions may be in the form of a lyophilized particulate material, a sterile or aseptically produced solution, a tablet, an ampoule, etc. Vehicles such as water (preferably buffered to a physiological pH such as PBS or other inert solid or liquid material may be present. In general, the compositions are prepared by being mixed with or dissolved in, bound to or otherwise combined with one of more water-insoluble or water-soluble aqueous or non aqueous vehicles, if necessary together with suitable additives and adjuvants. It is imperative that the vehicles and conditions shall not adversely affect the activity of the conjugate. Water as such is comprised within the expression vehicles.

A suitable therapeutic composition is used in the treatment of cancer of any kind including but not limited to carcinomas, sarcomas, lymphomas, leukemias and comprises a combination of:

(1) a recombinant DNA molecule encoding SAg in combination with, preferably fused with, another recombinant DNA sequence encoding another protein;

(2) a recombinant DNA molecule encoding SAg-in combination with another peptide or polypeptide; or

(3) a recombinant DNA molecule encoding a protein other than a SAg in combination with a SAg peptide or polypeptide.

These compositions that may comprise more than one components are administered together or sequentially and they may be combined (separately or together) with a delivery vehicle, preferably liposomes as disclosed herein.

Upon entering its intended or targeted cells, the therapeutic composition leads to the production of SAg and a second protein that may result in (a) apoptosis of the cancer cell and (b) with or without such apoptosis, the activation of effector cells of the immune system, including any or all of the following: cytotoxic T cells, NKT cells, NK cells, T helper cells and macrophages. The present therapeutic compositions are useful for the treatment of cancers, both primary tumors and tumor metastases.

Use of the present therapeutic composition overcomes the disadvantages of traditional treatments for metastatic cancer. For example. compositions of the present invention can target dispersed metastatic cancer cells that cannot be treated using surgery. In addition, administration of such compositions is not accompanied by the harmful side effects of conventional chemotherapy and radiotherapy.

A therapeutic composition also comprises a pharmaceutically acceptable carrier defined as any substance suitable as a vehicle for delivering a nucleic acid molecule (alone or in some combination with a protein) to a suitable in vivo or in vitro site. Preferred carriers are capable of maintaining DNA in a form that is capable of entering the target cell and being expressed by the cell.

Preferred carriers include: (1) those that transport, but do not specifically target a nucleic acid molecule to a cell (referred to herein as “non-targeting carriers”); and (2) those that deliver a nucleic acid molecule to a specific site in an animal or a specific cell (“targeting carriers”). Examples of non,targeting carriers are water, phosphate buffered saline (PBS), Ringer's solution, dextrose solution, serum-containing solutions, Hank's balanced salt solution, other aqueous, physiologically balanced solutions, oils, esters and glycols. Aqueous carriers can contain suitable additional substances which enhance chemical stability and isotonicity, such as sodium acetate, sodium chloride, sodium lactate, potassium chloride, calcium chloride, and other substances used to produce phosphate buffer, Tris buffer, and bicarbonate buffer and preservatives, such as thimerosal, m- and o-cresol, formalin and benzyl alcohol.

Preferred substances for aerosol delivery include surfactant substances such as esters or partial esters of fatty acids containing from about 6-22 carbon atoms. Examples are esters of caproic, octanoic. lauric, palmitic, stearic, linoleic, linolenic, olesteric, and oleic acids.

Other carriers can include metal particles (e.g., colloidal gold particles) for use with, for example, a biolistic gun through the skin.

Therapeutic compositions of the present invention can be sterilized by conventional methods and may be lyophilized.

The compositions of the present invention are delivered using a delivery vehicle that can be modified to target a particular site in a subject. Suitable targeting agents include ligands capable of selectively (i.e., specifically) binding to another molecule at a particular site. Examples are antibodies, antigens, receptors and receptor ligands. For example, an antibody specific for an antigen on the surface of a cancer cell can be placed on the outer surface of a liposome delivery vehicle to target the liposome to the cancer cell. By manipulating the chemical formulation of the lipid portion of a liposome preparation, it is possible to modulate its extracellular or intracellular targeting. For example, the charge of the lipid bilayer of a liposome surface can be varied chemically to promote fusion with cells having particular charge characteristics. Preferred liposomes comprise a compound that targets the liposome to a tumor cell, such as a ligand on the outer surface of the liposome that binds a molecule on the tumor cell surface.

Although the DNA constructs of the present invention can be administered in naked form, a liposome is a preferred vehicle for delivery in vivo. A liposome can remain stable in an animal for a sufficient amount of time, at least about 30 minutes, more preferably for at least about 1 hour and even more preferably for at least about 24 hours, to deliver a nucleic acid molecule to a desired site.

A liposome of the present invention comprises a lipid composition that can fuse with the plasma membrane of the targeted cell to deliver the encapsulated nucleic acid molecule into a cell. Preferably, the liposomes' transfection efficiency is about 0.5 mg DNA per 16 nmol of liposome delivered to about 10⁶ cells, more preferably about 1.0 mg DNA per 16 nmol of liposome delivered to about 10⁶ cells, and even more preferably about 2.0 mg DNA per 16 nmol of liposome delivered to about 10⁶ cells.

For use in the present invention, any liposome that is used in art-recognized gene delivery methods is appropriate. Preferred liposomes have a polycationic lipid composition and/or a cholesterol backbone conjugated to polyethylene glycol.

Complexing a liposome with nucleic acids for uses described herein is achieved using conventional methods. A suitable concentration of DNA to be added to a liposome preparation a concentration that is effective for delivering a sufficient amount of DNA molecules to a cell so that the cell can produce sufficient SAg and/or a other transduced protein to induce tumoricidal activity or to stimulate or regulate effector cells in a desired manner. Preferably, between about 0.1 mg and 10 mg of DNA is combined with about 8 nmol liposomes; more preferably, between about 0.5 mg and 5 mg of DNA is used even more preferably, about 1.0 mg of DNA is combined with about 8 nmol liposomes.

Another preferred delivery system is the sickled erythrocyte containing the nucleic acids of choice a given in Example 6. The sickled erythorcytes undergo ABO and RH phenotyping to select compatible cells for delivery. The cells are delivered intravenously or intrarterially in a blood vessel perfusing a specific tumor site or organ e.g. carotid artery, portal vein, femoral artery etc. over the same amount of time required for the infusion of a conventional blood transfusion. The quantity of cells to be administered in any one treatment would range from one tenth to one half of a full unit of blood. The treatments are generally given every three days for a total of twelve treaments. However, the treatment schedule is flexible and may be given for a longer of shorter duration depending upon the patients response.

Another preferred delivery vehicle is a recombinant virus particle, for example, in the form of a vaccine. A recombinant virus vaccine of the present invention includes the DNA encoding the therapeutic composition packaged in a viral coat that allows entrance of the transducing DNA into a cell and its expression. A number of recombinant virus particles can be used, for example, alphaviruses, poxviruses, adenoviruses, herpesviruses, arena virus and retroviruses.

Also useful as a delivery vehicle is a “recombinant cell vaccine,” preferably tumor vaccines, in which allogeneic (though histocompatible) or autologous tumor cells are transfected with a DNA preparation encoding the therapeutic proteins or peptides to be expressed. The cells are preferably irradiated and then administered to a patient by any of a number of known injection routes.

The therapeutic compositions that are administered by “tumor cell vaccine,” includes the recombinant molecules without carrier. Treatment with tumor cell vaccines is useful for primary or localized tumors as well as metastases. When used to treat metastatic cancer, which includes prevention of further metastatic disease, as well as, the cure existing metastatic disease,

As used herein, the term “treating” a disease includes alleviating the disease or any of its symptoms and/or preventing the development of a secondary disease resulting from the occurrence of the initial disease.

An “effective treatment protocol” includes a suitable and effective dose of an agent being administered to a subject, given by a suitable route and mode of administration to achieve its intended effect in treating a disease.

Effective doses and modes of administration for a given disease can be determined by conventional methods and include, for example, determining survival rates, side effects (i.e., toxicity) and qualitative or quantitative, objective or subjective, evaluation of disease progression or regression. In particular, the effectiveness of a dose regimen and mode of administration of a therapeutic composition of the present invention to treat cancer can be determined by assessing response rates. A “response rate” is defined as the percentage of treated subjects that responds with either partial or complete remission. Remission can be determined by, for example, measuring tumor size or by microscopic examination of a tissue sample for the presence of cancer cells.

In the treatment of cancer, a suitable single dose can vary depending upon the specific type of cancer and whether the cancer is a primary tumor or a metastatic form. One of skill in the art can test doses of a therapeutic composition suitable for direct injection to determine appropriate single doses for systemic administration, taking into account the usual subject parameters such as size and weight. An effective anti-tumor single dose of a therapeutic recombinant DNA molecule or combination thereof is an amount sufficient amount to result in reduction, and preferably elimination, of the tumor after the DNA molecule or combination has transfected cells at or near the tumor site.

A preferred single dose of SAg-encoding DNA molecule or fusion product thereof is an amount that, when transfected into a target cell population, leads to the production of SAg in an amount, per transfected cell, ranging from about 250 femtograms (fg) to about 1 mg, preferably from about 500 fg to about 500 pg and more preferably from about 1 pg to about 100 pg.

When the SAg-encoding DNA is combined with a second DNA molecule encoding a second protein product, an effective single dose of a the second DNA molecule is an amount that when transfected into a target cell population leads to the production of the second protein product in an amount, per transfected cell, ranging from about 10 fg to about 1 mg, more preferably from about 100 fg to about 750 pg.

An effective cancer-treating single dose of SAg-encoding DNA and a second DNA molecule encoding a second protein when administered to a subject using a non-targeting carrier, is an amount capable of reducing, and preferably eliminating, the primary or metastatic tumor following transfection by the recombinant molecules of cells at or near the tumor site. A preferred single dose of such a therapeutic composition is from about 100 mg to about 4 mg of total recombinant DNA, more preferably from about 200 mg to about 2 mg, most preferably from about 200 mg to about 800 mg of total recombinant molecules.

A preferred single dose of liposome-complexed, SAg-encoding DNA, is from about 100 mg of total DNA per 800 nmol of liposome to about 4 mg of total DNA molecules per 32 mmol of liposome, more preferably from about 200 mg per 1.6 mmol of liposome to about 3 mg of total recombinant DNA per 24 mmol of liposome., and even more preferably from about 400 mg per 3.2 mmol of liposome to about 2 mg per 16 mmol of liposome.

One of skill in the art recognizes that the number of doses required depends upon the extent of disease and the response of an individual to treatment. Thus, according to this invention, an effective number of doses includes any number required to cause regression of primary or metastatic disease.

A preferred treatment protocol comprises monthly administrations of single doses (as described above) for up to about 1 year. An effective number of doses (per individual) of a SAg-encoding DNA molecule and a second DNA molecule encoding a second protein, when administered in a non-targeting carrier or when complexed with liposomes, is from about 1 to about 10 dosings, preferably from about 2 to about 8 dosings, and even more preferably from about 3 to about 5 dosings. Preferably, such dosings are administered about once every 2 weeks until signs of remission appear, followed by about once a month until the disease is gone.

The therapeutic compositions can be administered by any of a variety of modes and routes, including but not limited to, local administration into a site in the subject animal, which site contains abnormal cells to be destroyed. An example is the local injection within the area of a tumor or a lesion. Another example is systemic administration.

Therapeutic compositions that are best delivered by local administration include recombinant DNA molecules

(a) in a non-targeting carrier (e.g., “naked” DNA molecules as taught in Wolff K et al., 1990, Science 247, 1465-1468); and

(b) complexed to a delivery vehicle.

Suitable delivery vehicles for local administration include liposomes, and may further comprise ligands that target the vehicle to a particular site.

A preferred mode of local administration is by direct injection. Direct injection techniques are particularly useful for injecting the composition into a cellular or tissue mass such as a tumor mass or a granuloma mass that has been induced by a pathogen. Thus, the present recombinant DNA molecule complexed with a delivery vehicle is preferably injected directly into, or locally in the area of, a tumor mass or a single cancer cell.

The present composition may also be administered in or around a surgical wound. For example, a patient undergoes surgery to remove a tumor. Upon removal of the tumor, the therapeutic composition is coated on the surface of tissue inside the wound or injected into areas of tissue inside the wound. Such local administration will treat cancer cells that were not successfully removed by the surgical procedure, as well as prevent recurrence of the primary tumor or development of a secondary tumor in the surgical area.

Therapeutic compositions that are best delivered by systemic administration include recombinant DNA molecules complexed to a tumor binding ligand or a ligand that binds to the tumor vasculature or stroma. Examples are antibodies, antigens, receptor, receptor ligand or a targeted delivery vehicle as disclosed herein. These delivery vehicles may be liposomes into which are incorporated targeting ligands, preferably ligands that targeting the vehicle to the site of tumor cells or another type of lesion. For cancer treatment, ligands that selectively bind to cancer cells, or to cells within the area of a cancer cell, are preferred. Systemic administration is used to treat primary or localized tumors and, in particular, tumor metastases wherein the cancer cells are dispersed. Systemic administration is advantageous when targeting cancer in organs, especially those difficult to reach for direct injection, (e.g., heart, spleen, lung or liver).

Preferred modes and routes of systemic administration include intravenous injection and aerosol, oral and percutaneous (topical) delivery. Intravenous injection methods and aerosol delivery are performed conventionally. Oral delivery is achieved preferably by complexing the therapeutic composition to a carrier capable of withstanding degradation by digestive enzymes in the subject's digestive system. Examples of such carriers, includes plastic capsules or tablets as are known in the art. For topical delivery, the therapeutic composition is mixed with a lipophilic reagent (e.g., DMSO) that can pass into the skin.

The therapeutic compositions and methods of the present invention are intended for animals, preferably mammals and birds, in particular house pets, farm animals and zoo animals as these terms are generally understood. By “farm animals” are intended animals that are eaten or those that produce useful products (e.g., wool-producing sheep). Examples of preferred animal subjects to be treated are dogs, cats, sheep, cattle, horses and pigs. The present compositions and methods are effective in inbred and outbred animal species. Most preferably, the animal is a human.

Another component useful in combination with the therapeutic nucleic acids of this invention is an adjuvant suited for use with a nucleic acid-based vaccine. Examples of adjuvant-containing compositions include

1) SAg-encoding DNA and a second DNA encoding a recombinant protein; or

2) SAg-encoding DNA combined with another peptide or polypeptide; or

3) DNA encoding a second recombinant protein and a SAg peptide or polypeptide.

As indicated above, effective doses of a SAg-encoding DNA combined with a second DNA molecule, or a vaccine nucleic acid molecule are determined conventionally by those skilled in the art. One measure of an effective dose is that produces a sufficient amount of SAg and second protein to stimulate effector cell immunity in a manner that enhances the effectiveness of the vaccine. Adjuvants of the present invention are particularly suited for use in humans because many traditional adjuvants (e.g., Freund's adjuvant and other bacterial cell wall components) are toxic whereas others are relatively ineffective (e.g., aluminum-based salts and calcium-based salts).

### EXAMPLE 15

General Procedures for In Vivo and Ex Vivo Sensitization to Produce Tumor Specific Effector Cells for Adoptive Immunotherapy

Tumor growth is initiated by subcutaneous inoculation of mice on both flanks with 1.5×10⁶ tumor cells suspended in 0.05 ml of HBSS. After 9-12 days of tumor growth (approximately 8 mm in diameter), tumor-draining inguinal LN are removed sterilely. Lymphocyte suspensions are prepared by teasing LN with needles followed by pressing with the blunt end of a 10-ml plastic syringe in HBSS. Tumor draining LN cells are stimulated in vitro in a two-step procedure. Briefly, 4×10⁶ LN cells in 2 ml of complete medium (CM) containing the SAg constructs are incubated in a well of 24-well plates at 37° C. in a 5% CO2 atmosphere for 2 days. CM consisted of RPMI 1640 medium supplemented with 10% heat-inactivated FCS, 0.1 mM nonessential amino acids, 1 mM sodium pyruvate, 2 mM freshly prepared L-glutamine. 100 mg/ml streptomycin, 100 U/ml penicillin, a 50 mg/ml gentamycin, 0.55 mg/ml fungizone (all from GIBCO, Grand Island, N.Y.) and 5×10⁻⁵ M 2-mercaptoethanol (Sigma). The cells were harvested, then washed and further cultured a 3×10⁵/well in 2 ml of CM with IL-2. After 3-day incubation in IL-2, the cells are collected and counted to determine the degree of proliferation. Finally, the cells are suspended in appropriate media for flow cytometric analysis, evaluation of cytotoxicity and lymphokine secretion, or for adoptive immunotherapy.

### EXAMPLE 16

General Adoptive Immunotherapy Protocol

Mice are injected with 2 to 3×10⁵ syngeneic tumor cells suspended in 1 ml of HBSS to initiate pulmonary metastases. On day 3, activated cells are given i.v. at numbers indicated generally 10⁶-10⁷. In some instances, mice are also treated with 15,000 U IL2 in 0.5 ml HBSS twice daily for 4 consecutive days to promote the in vivo function and survival of the activated cells. On day 20 or 21, all mice are randomized, killed and metastatic tumor nodules on the surface of the lungs enumerated as previously described. If pulmonary metastases exceeded 250, this number is arbitrarily assigned for statistical analysis. The significance of differences in metastases numbers between experimental group is determined by the Wilcoxon rank sum test. Two sided p values of <0.1 are considered significant. Each experimental group consists of at least five mice and no animal was excluded from the statistical evaluation.

For testing SAg-glycosylceramide complexes and SAg lipopolysaccharide complexes, additional models are used to assess the dependence of the antitumor effect on NKT cells. Natural killer T cells (NKT) lymphocytes express an invariant TCR encoded by the Vα 14 and Ja281 gene segments. Mice with a deletion of Ja281 exclusively lack Vα 14. The Vα 14 NKT cell-deficient mice no longer mediate IL-12 induced rejection of tumors.

Also generated are transgenic mice lacking recombination activating gene (RAG) which preferentially generate Vα 14 NKT cells but block the development of other lymphocyte lineages, including NK, B, and T cells. These mice are termed Vα 14 NKT mice. J281+/+(wild type), J281−/−(deleted of V14) and RAG−/−V14 tgV8.2 tg (deleted of NK, T and B cells but preferentially generate Vα 14 NKT cells) mice are injected

(a) with 2×10⁶ B16 or FBL-3 (erythroleukemia) cells in the spleen to induce liver metastasis,

(b) intravenously with 3×10⁵ B16 or 2×10⁶ LLC (Lewis lung carcinoma) cells for pulmonary metastases or

(c) subcutaneously with 2×10⁶ B16 cells (melanoma) for subcutaneous tumor growth on day 0.

SAg conjugates or fusion proteins are injected in doses of 0.1 to 50 mg on days 3, 5, 7, and 9 after the day of tumor implantation. Control animals are injected with PBS on the same schedule. On day 14, the mice are killed and either metastatic nodules counted or GM3 melanoma antigens measured by radioimmunoassay as previously described. For subcutaneous tumor growth, injection of IL-12 or PBS is initiated on day 5, and the mice are treated five times per week. The diameters of tumors are measured daily with calipers. The sizes of the tumor are expressed as the products of the longest diameter times the shortest diameter (in mm²).

### EXAMPLE 17

Preparation and Administration of DNA Liposome Complexes

A representative protocol for administration of DNA-liposome complexes is as follows: DNA liposome complexes are mixed immediately prior to injection by adding 0.1 ml of lactated Ringer's solution into a sterile vial of plasmid DNA (20 mg/ml; 0.1 ml). An aliquot of this solution (0.1 ml) is added at room temperature to 0.1 ml of 150 mM (dioleoyl phosphatidylethanolamine/3β[N—(N′,N′-dimethylaminoethane)-carbamoyl]cholesterol) liposome in lactated Ringer's solution in a separate sterile vial. The DNA and liposome vials are prepared in accordance with FDA guidelines and quality control procedures. After incubation for 15 minutes at room temperature, an additional 0.5 ml of sterile lactated Ringer's solution is added to the vial and mixed. The DNA liposome solution (0.2 ml) is injected into the patient's tumor nodule under sterile conditions at the bedside after administration of local anesthesia (1% lidocaine) using a 22-gauge needle. For catheter delivery, the DNA liposome solution (0.6 ml) is delivered into the artery using percutaneous delivery. Additional protocols for administration of DNA liposomal constructs are given in Nabel, G J, Methods for Liposome-Mediated Gene Transfer to Tumor Cells in vivo, in: Methods in Molecular Medicine, Gene Therapy Protocols, Robbins P ed. Humana Press, Totowa N.J. (1996). Cationic liposomes for delivery of DNA construct to the tumor endothelium are prepared by the method of Thurston et al., J. Clin Invest., 101: 1401-1413, (1998).

### EXAMPLE 18

General Procedures for Administering Constructs in Human Tumor Models and Human Patients

The constructs described herein are tested for therapeutic efficacy in several well established rodent models which are considered to be highly representative as described in “Protocols for Screening Chemical Agents and Natural Products Against Animal Tumors and Other Biological Systems (Third Edition)”, Cancer Chemother. Reports, Part 3, 3: 1-112, which is hereby incorporated by reference in its entirety. Additional tumor models of carcinoma and sarcoma originating from primary sites and prepared as established tumors at primary and/or metastatic sites are utilized to test further the efficacy of the constructs.

### EXAMPLE 19

General Procedures for Administering Tumor Cells or Sickled Erythrocytes Transduced with SAgs and SAg-Activated T or NKT Cells in Human Tumor Models and Human Patients

A. Tumor Cells Transduced with SAg Nucleic Acids Alone or Cotransfected with Oncogenes or Nucleic Acids Encoding Potent Immunogens and Bacterial Products

In a representative protocol, using the B16 melanoma or A20 lymphoma or other models given above, 10⁵-10⁷ transfected tumor cells are implanted subcutaneously and 1-6 months later 10⁵-10⁷ untransfected tumor cells, are implanted. In the case of tumor cells cotransfected with several therapeutic nucleic acids, controls are established consisting of groups transfected with only one of the nucleic acids. These single transfectants are administered on the same schedule as the cotransfectants and assessed for capacity to prevent or reverse tumor growth compared to positive controls receiving tumor alone. The animals receiving the SAg transfected tumor cells show no evidence of growth of the wild type tumor and prolonged survival compared to the controls in which there is 100% appearance of the tumors. The differences are statistically significant.

SAg transfected tumor cells are also used to treat established tumors as follows. Transfected tumor cells, 10⁵-10⁷ are given 3-10 days after the appearance of established tumors. Results show statistically significant arrest of tumor growth, prolongation of survival in treated animals compared to untreated controls.

B. SAP Activated Effector T or NKT Cells

Effector T or NKT cells are generated as described elsewhere and are infused intravenously in doses of 10⁶-10⁸ into syngeneic hosts that have pulmonary metastatic lesions established by injecting tumor cells intravenously 3 to 12 days earlier. Twenty days later, the animals are sacrificed and pulmonary metastases measured in treated animals compared to untreated controls. Results show statistically significant reduction in total number of pulmonary nodules and prolonged survival in the treated group compared to untreated controls.

### EXAMPLE 20

General Test Evaluation Procedures for Constructs and SAg Activated Effector T or NKT Cells

I. General Test Evaluation Procedures

A. Calculation of Mean Survival Time

Mean survival time is calculated according to the following formula:

$$\text{Mean survival time (days)} = \frac{S + AS ( A - 1 ) - ( B + 1 ) N T}{S ( A - 1 ) - N T}$$

Definitions:

Day: Day on which deaths are no longer considered due to drug toxicity. Example: with treatment starting on Day 1 for survival systems (such as L1210, P388, B16, 3LL, and W256):

Day A: Day 6.

Day B: Day beyond which control group survivors are considered “no-takes.”

Example: with treatment starting on Day 1 for survival systems (such as L1210, P388, and W256), Day B-Day 18. For B16, transplanted AKR, and 3LL survival systems, Day B is to be established.

S: If there are “no-takes” in the treated group, S is the sum from Day A through Day B. If there are no “no-takes” in the treated group, S is the sum of daily survivors from Day A onward.

S(A−1): Number of survivors at the end of Day (A−1).

Example: for 3LE21, S(A−1)=number of survivors on Day 5.

NT: Number of “no-takes” according to the criteria given in Protocols 7.300 and 11.103.

B. T/C Computed for all Treated Groups

T/C is the ratio (expressed as a percent) of the mean survival time of the treated group divided by the mean survival time of the control group. Treated group animals surviving beyond Day B, according to the chart below, are eliminated from calculations:

| No. of survivors in | Percent of “no-takes” | |
|---|---|---|
| treated group beyond Day B | in control group | Conclusion |
| 1 | Any percent | “no-take” |
| 2 | <10 | drug inhibition |
| | 10 | “no-takes” |
| 3 | <15 | drug inhibition |
| | 15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

Calculation of Median Survival Time

Median Survival Time is defined as the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of Median Survival Time From Survivors

If the total number of animals including survivors (N) is even, the median survival time (days) (X+Y)/2, where X is the earlier day when the number of survivors is N/2, and Y is the earliest day when the number of survivors (N/2)−1. If N is odd, the median survival time (days) is X.

D. Computation of Median Survival Time From Mortality Distribution

If the total number of animals including survivors (N) is even, the median survival time (days) (X+Y)/2, where X is the earliest day when the cumulative number of deaths is N/2, and Y is the earliest day when the cumulative number of deaths is (N/2)+1. If N is odd, the median survival time (days) is X.

Cures and “No-Takes”: “Cures” and “no-takes” in systems evaluated by median survival time are based upon the day of evaluation. On the day of evaluation any survivor not considered a “no-take” is recorded as a “cure.” Survivors on day of evaluation are recorded as “cures” or “no-takes,” but not eliminated from the calculation of the median survival time.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$\text{Tumor weight (mg)} = \frac{length ( mm ) \times {( width [ mm ] )}^{2}}{2} \text{Or}$$

$$\frac{L \times {( W )}^{2}}{2}$$

The reporting of tumor weights calculated in this way is acceptable inasmuch as the assumptions result in as much accuracy as the experimental method warrants.

F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight From Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of small | Percent of | |
|---|---|---|
| tumors in | “no-takes” in | |
| treated group | control group | Action |
| 17 | Any percent | no-take; not used in |
| | | calculations |
| 18-39 | <10 | drug inhibition; use in |
| | | calculations |
| | 10 | no-takes; not used in |
| | | calculations |
| 40 | <15 | drug inhibition; use in |
| | | calculations |
| | 15 | Code all nontoxictests |
| | | “33” |

Positive control compounds are not considered to have “no-takes” regardless of the number of “no-takes” in the control group. Thus, the tumor weights of all surviving animals are used in the calculation of T/C for the positive control. T/C are computed for all treated groups having more than 65% survivors. The T/C is the ratio (expressed as a percent) of the mean tumor weight for treated animals divided by the mean tumor weight for control animals. SDs of the mean control tumor weight are computed the factors in a table designed to estimate SD using the estimating factor for SD given the range (difference between highest and lowest observation). Biometrik Tables for Statisticians (Pearson E S, and Hartley H G, eds.) Cambridge Press, vol. 1, table 22, p. 165.

II. Specific Tumor Models

A. Lymphoid Leukemia L1210

Summary: Ascitic fluid from donor mouse is transferred into recipient BDF1 or CDF1 mice. Treatment begins 24 hours after implant. Results are expressed as a percentage of control survival time. Under normal conditions, the inoculum site for primary screening is i.p., the composition being tested is administered i.p., and the parameter is mean survival time. Origin of tumor line: induced in 1948 in spleen and lymph nodes of mice by painting skin with MCA. J Natl Cancer Inst. 13:1328, 1953.

Animals

Propagation: DBA/2 mice (or BDF1 or CDF1 for one generation).

Testing: BDF1 (C57BL/6×DBA/2) or CDF1 (BALB/c x DBA/2) mice.

Weight: Within a 3-g weight range, with a minimum weight of 18 g for males and 17 g for females.

Sex: One sex used for all test and control animals in one experiment.

Experiment Size: Six animals per test group.

Control Groups: Number of animals varies according to number of test groups.

Tumor Transfer

Inject i.p., 0.1 ml of diluted ascitic fluid containing 10⁵ cells.

Time of Transfer for Propagation: Day 6 or 7.

Time of Transfer for Testing: Day 6 or 7.

Testing Schedule

Day 0: Implant tumor. Prepare materials. Run positive control in every odd-numbered experiment. Record survivors daily.

Day 1: Weigh and randomize animals. Begin treatment with therapeutic composition.

Typically, mice receive 1 ug of the test composition in 0.5 ml saline. Controls receive saline alone. The treatment is given as one dose per week. Any surviving mice are sacrificed after 4 weeks of therapy.

Day 5: Weigh animals and record.

Day 20: If there are no survivors except those treated with positive control compound, evaluate study.

Day 30: Kill all survivors and evaluate experiment.

Quality Control

Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%

Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

B. Lymphocytic Leukemia P388

Summary: Ascitic fluid from donor mouse is implanted in recipient BDF1 or CDF1 mice. Treatment begins 24 hours after implant. Results are expressed as a percentage of control survival time. Under normal conditions, the inoculum site for primary screening is ip, the composition being tested is administered ip daily for 9 days, and the parameter is median survival time. Origin of tumor line: induced in 1955 in a DBA/2 mouse by painting with MCA. Scientific Proceedings, Pathologists and Bacteriologists 33:603, 1957.

Animals

Propagation: DBA/2 mice (or BDF1 or CDF1 for one generation)

Testing: BDF1 (C57BL/6×DBA/2) or CDF1 (BALB/c×DBA/2) mice.

Weight: Within a 3-g weight range, with a minimum weight of 18 g for males and 17 g for females.

Sex: One sex used for all test and control animals in one experiment.

Experiment Size: Six animals per test group.

Control Groups: Number of animals varies according to number of test groups.

Tumor Transfer

Implant: Inject ip

Size of Implant: 0.1 ml diluted ascitic fluid containing 10⁶ cells.

Time of Transfer for Propagation: Day 7.

Time of Transfer for Testing: Day 6 or 7.

Testing Schedule

Day 0: Implant tumor. Prepare materials. Run positive control in every odd-numbered experiment. Record survivors daily.

Day 1: Weigh and randomize animals. Begin treatment with therapeutic composition.

Typically, mice receive 1 ug of the composition being tested in 0.5 ml saline. Controls receive saline alone. The treatment is given as one dose per week. Any surviving mice are sacrificed after 4 weeks of therapy.

Day 5: Weigh animals and record.

Day 20: If there are no survivors except those treated with positive control compound, evaluate experiment.

Day 30: Kill all survivors and evaluate experiment.

Quality Control

Acceptable median survival time is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a synthetic must have two multi-dose assays (each performed at a different laboratory) that produce a T/C 125%; a natural product must have two different samples that produce a T/C 125% in multi-dose assays.

C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The composition being tested is administered ip, and the parameter is mean survival time. Origin of tumor fine: arose spontaneously in 1954 on the skin at the base of the ear in a C57BL/6 mouse. Handbook on Genetically Standardized Jax Mice. Roscoe B. Jackson Memorial Laboratory, Bar Harbor, Maine, 1962. See also Ann NY Acad Sci 100, Parts 1 and 2, 1963.

Animals

Propagation: C57BL/6 mice.

Testing: BDF1 (C57BL/6×DBA/2) mice.

Weight: Within a 3-g weight range, with a minimum weight of 18 g for males and 17 g for females.

Sex: One sex used for all test and control animals in one experiment.

Experiment Size: Ten animals per test group. For control groups, the number of animals varies according to number of test groups.

Tumor Transfer

Propagation: Implant fragment sc by trochar or 12-gauge needle or tumor homogenate (see below) every 10-14 days into axillary region with puncture in inguinal region.

Testing: Excise sc tumor on Day 10-14.

Homogenate: Mix 1 g or tumor with 10 ml of cold balanced salt solution and homogenize, and implant 0.5 ml of this tumor homogenate ip or sc.

Fragment: A 25-mg fragment may be implanted sc.

Testing Schedule

Day 0: Implant tumor. Prepare materials. Run positive control in every odd-numbered experiment. Record survivors daily.

Day 1: Weigh and randomize animals. Begin treatment with therapeutic composition.

Typically, mice receive 1 mg of the composition being tested in 0.5 ml saline. Controls receive saline alone. The treatment is given as one dose per week. Any surviving mice are sacrificed 8 weeks of therapy.

Day 5: Weigh animals and record.

Day 60: Kill all survivors and evaluate experiment.

Quality Control

Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

Evaluation

Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a therapeutic composition should have two multi-dose assays that produce a T/C 125%.

Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. The treatment is given as one dose per week. Mice sacrificed after 4 weeks of therapy, the lungs are removed and metastases are enumerated.

C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. The composition being tested is administered ip daily for 11 days and the results are expressed as a percentage of the control.

Origin of tumor line: arose spontaneously in 1951 as carcinoma of the lung in a C57BL/6 mouse. Cancer Res 15:39, 1955. See, also Malave, I. et al., J. Nat'l. Canc. Inst. 62:83-88 (1979).

Animals

Propagation: C57BL/6 mice.

Testing: BDF1 mice or C3H.

Weight: Within a 3-g weight range, with a minimum weight of 18 g for males and 17 g for females.

Sex: One sex used for all test and control animals in one experiment.

Experiment Size: Six animals per test group for sc implant, or ten for im implant. For control groups, the number of animals varies according to number of test groups.

Tumor Transfer

Implant: Inject cells im in hind leg or implant fragment sc in axillary region with puncture in inguinal region.

Time of Transfer for Propagation: Days 12-14.

Time of Transfer for Testing: Days 12-14.

Testing Schedule

Day 0: Implant tumor. Prepare materials. Run positive control in every odd-numbered experiment. Record survivors daily.

Day 1: Weigh and randomize animals. Begin treatment with therapeutic composition.

Typically, mice receive lug of the composition being tested in 0.5 ml saline. Controls receive saline alone. The treatment is given as one dose per week. Any surviving mice are sacrificed after 4 weeks of therapy.

Day 5: Weigh animals and record.

Final Day: Kill all survivors and evaluate experiment.

Quality Control

Acceptable im tumor weight on Day 12 is 500-2500 mg. Acceptable im tumor median survival time is 18-28 days. Positive control compound is cyclophosphamide: 20 mg/kg/injection, qd, Days 1-11. Check control deaths, no takes, etc.

Evaluation

Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity a synthetic must have two multi-dose assays (each performed at a different laboratory); a natural product must have two different samples.

D. 3LL Lewis Lung Carcinoma Metastasis Model

This model has been utilized by a number of investigators. See, for example, Gorelik, E. et al., J. Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J. E. et al., J. Nat'l. Canc. Inst. 69:975-980 (1982); Hilgard, P. et al., Br. J.Cancer 35:78-86(1977)).

Mice: male C57BL/6 mice, 2-3 months old.

Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days.

Typically, mice receive lug of the composition being tested in 0.5 ml saline. Controls receive saline alone. The treatment is given as one or two doses per week. In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M. L. et al., J. Lab. Clin. Med. 89:217-228 (1977). Ten days following tumor amputation, 25 mg of FdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 1×10⁶ 3LL cells. Amputation of tumors produced following inoculation of 1×10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴−1×10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

E. Walker Carcinosarcoma 256

Summary: Tumor may be implanted sc in the axillary region as a 2-6 mm fragment, im in the thigh as a 0.2-ml inoculum of tumor homogenate containing 10⁶ viable cells, or ip as a 0.1-ml suspension containing 10⁶ viable cells. Treatment of the composition being tested is usually ip. Origin of tumor line: arose spontaneously in 1928 in the region of the mammary gland of a pregnant albino rat. J Natl Cancer Inst 13:1356, 1953.

Animals

Propagation: Random-bred albino Sprague-Dawley rats.

Testing: Fischer 344 rats or random-bred albino rats.

Weight Range: 50-70 g (maximum of 10-g weight range within each experiment).

Sex: One sex used for all test and control animals in one experiment.

Experiment Size: Six animals per test group. For control groups, the number of animals varies according to number of test groups.

Time of Tumor Transfer

Time of Transfer for Propagation: Day 7 for im or ip implant; Days 11-13 for sc implant.

Time of Transfer for Testing: Day 7 for im or ip implant; Days 11-13 for sc implant.

Tumor Transfer

Sc fragment implant is by trochar or 12-gauge needle into axillary region with puncture in inguinal area. Im implant is with 0.2 ml of tumor homogenate (containing 10⁶ viable cells) into the thigh. Ip implant is with 0.1 ml of suspension (containing 10⁶ viable cells) into the ip cavity.

Testing Schedule

Prepare and administer compositions under test on days, weigh animals, and evaluate test on the days listed in the following tables.

| | Prepare | Administer | Weigh | |
|---|---|---|---|---|
| Test system | drug | drug | animals | Evaluate |
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

Day 0: Implant tumor. Prepare materials. Run positive control in every odd-numbered experiment. Record survivors daily.

Day 1: Weigh and randomize animals.

Final Day: Kill all survivors and evaluate experiment.

Quality Control

Acceptable im tumor weight or survival time for the above three test systems: 5WA16: 3-12 g. 5WA12: 3-12 g. 5WA31 or 5WA21: 5-9 days.

Evaluation

Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity a therapeutic agent must have activity in two multi-dose assays.

F. A20 Lymphoma

10⁶ murine A20 lymphoma cells in 0.3 ml saline are injected subcutaneously in Balb/c mice. The mice are treated intravenously with 1 g of the composition being tested in 0.5 ml saline. Controls receive saline alone. The treatment is given as one dose per week. Tumor growth is monitored daily by physical measurement of tumor size and calculation of total tumor volume. After 4 weeks of therapy the mice are sacrificed.

Treatment Regimens and Results (Constructs)

For determining efficacy in the tumor models described above the general categories of therapeutic constructs used are given below. For all of the classes of conjugates listed below, the SAg component can be prepared as either a DNA encoding SAg or as the SAg polypeptide itself. In either form the SAg DNA or protein may be conjugated to additional molecules, either nucleic acid or polypeptides. Operationally, for therapeutic use in vivo or ex vivo, these conjugates may be prepared by chemical coupling or by recombinant means (whichever is appropriate) and conjugated to a tumor-targeting structure or incorporated into a vehicle (e.g., liposomes) that themselves comprise a tumor targeting structure(s). Again, examples of such targeting structures include, but are not limited to, an antibody, antigen, receptor or receptor ligand. Methods are disclosed in Examples 1, 3, 4, 5, 6, 7, 14, 17, 18, 30-32.

1. SAg Nucleic Acid Constructs including Phage Displays and SAg Transfected Bacterial Cells

2. Glycosylated SAgs

3. Chimeric SAgs

Conjugates having a Superantigen component (polypeptide or nucleic acid) and a partner that is either a single component or a conjugate of 2 or more components (protein, carbohydrate, lipid or DNA) as indicated below.

| Superantigen (Protein or DNA) | Partner (Single Component or Conjugate) |
|---|---|
| 4. | DNA coding sequence |
| 5. | Polypeptide |
| 6. | Nucleic acid |
| 7. | Tumor associated Peptide |
| 8. | Tumor Antigen-MHC protein |
| 9. | LPS |
| 10. | Lipoarabinomannan |
| 11. | Ganglioside |
| 12. | Glycosphingolipid |
| 13. | Ganglioside-CD1 receptor |
| 14. | Glycosphingolipid-CD1 receptor |
| 15. | Glycosylceramide (e.g., Gal-Cer) |
| 16. | GalCer-CD1 receptor |
| 17. | Gal |
| 18. | Arg-Gly-Asp or Asn-Gly-Arg |
| 19 | iNOS |
| 20. | Gb2 or Gb3 or Gb4 |
| 21. | (Gb2 or Gb3 or Gb4)-CD1 receptor |
| 22. | -GPI-(Gb2 or Gb3 or Gb4) |
| 23. | -GPI-(Gb2 or Gb3 or Gb4)-CD1 receptor |
| 24. | Verotoxin |
| 25. | Verotoxin A or B Subunit_{—} |
| 26. | IFNα receptor peptide homologous to VT |
| 27. | CD19 peptide homologous to VT |
| 28. | LDL, VLDL, HDL, IDL |
| 29. | Apolipoproteins (e.g., Lp(a), apoB-100, apoB-48, apoE) |
| 30. | OxyLDL, oxyLDL mimics, (e.g., 7β-hydroperoxycholesterol, |
| | 7β-hydroxycholesterol, 7-ketocholesterol, 5α-6α- |
| | epoxycholesterol, 7β-hydroperoxy-choles-5-en-3β-ol, 4- |
| | hydroxynonenal (4-HNE), 9-HODE, 13-HODE and |
| | cholesterol-9-HODE) |
| 31. | OxyLDL by products (e.g. lysolecithin, |
| | lysophosphatidylcholine, malondialdehyde, 4- |
| | hydroxynonenal) |
| 32. | LDL & oxyLDL receptors (e.g., LDL oxyLDL, acetyl-LDL, |
| | VLDL, LRP, CD36, SREC, LOX-1, macrophage scavenger |
| | receptors) |
| 33. | phytosphingosine, -GPI-phytosphingosine |
| 34. | tumor associated lipid antigens |
| 35. | glycolipid, proteolipid, glycosphingolipid, sphingolipid |
| | with inositolphosphate -containing head groups, |
| | phytoglycolipids, mycoglycolipids, -GPI-sphingosines, |
| | -GPI-lipids |
| 36. | sphingolipids with inositolphosphate-containing head |
| | groups having the general structure: |
| | ceramide-P-myoinositol-X with X referring to polar |
| | substituents comprising ceramide-p-inositol-mannose, |
| | inositol-1-P-(6)mannose(a1,2 inositol-1P-(1)ceramide, |
| | (inositol-P)2-ceramide, inositol-P-inositol-P-ceramide, |
| | inositol-P-inositol-P-ceramide. |
| 37. | tumor associated glycan antigens consisting of |
| | peptidoglycans or glycan phosphotidyinositol (GPI) |
| | structures |

Vaccine Use

For use as a vaccine, the constructs are administered subcutaneously, intramuscularly intradermally or intraperitoneally in doses ranging from 50 to 500 ng in various vehicles such as Freund's adjuvant, aluminum hydroxide, pluruonic acid triblock and liposomes as described in the art. Doses may be repeated every 10 days. Tumors are implanted after the last dose. A control group does not receive the vaccine.

Use in Established Tumors

For proteins or nucleic acid constructs, treatment consists of injecting animals iv or ip with 50, 500 1000 or 5,000 ng of in 0.1-0.5 ml of normal saline. Unless indicated otherwise above, treatments are given one to three times per week for two to five weeks. Phage displays, yeast displays and vesilcle, SAg-bacterial or viral constructs or SAg vesicles are administered as 10⁹ transducing units (TU) and irradiated bacterial cells, yeast cells as 10⁵-10⁶ cells iv into the tail vein one to three times per week for two to five weeks or directly into tumor in 30-75% or the iv doses on the same schedule. Exosomes or vesicles, harvested from transfected, transformed or fusion tumor cells or sickled cells or mutant yeast are given i.v. into the tail vein in a dose of 0.25-1 g per animal one to three times per week for two to five weeks. The results shown in Table VI are for each composition and dose tested. The results are statistically significant by the Wilcoxon rank sum test.

Treatment regimens for SAg activated effector T or NKT cells are in Example 16, 18, 19. The preferred animal model for evaluation of the adoptively transferred T or NKT effector cells is the MCA 205/207 fibrosarcoma with pulmonary metastases (Shu S. et al., J. Immunol. 152: 1277-1288 (1994)). The other models given in Example 20 are also suitable for evaluation of the therapeutic effectiveness of the effector T cells.

TABLE VI

| Tumor Model | Parameter | % of Control Response |
|---|---|---|
| L1210 | Mean survival time | >130% |
| P388 | Mean survival time | >130% |
| B16 | Mean survival time | >130% |
| B16 metastasis | Median number of metastases | <70% |
| 3LL | Mean survival time | >130% |
| | Mean tumor weight | <40% |
| 3LL metastasis | Median survival time | >130% |
| | Mean lung weight | <60 |
| | Median number of metastases | <60% |
| | Median volume of metastases | <60% |
| | Medial volume of metastases | <60% |
| | Median uptake of IdUrd | <60% |
| Walker carcinoma | Median survival time | >130% |
| | Mean tumor weight | <40% |
| A20 | Mean survival time | >130% |
| | Mean tumor volume | <40% |

### EXAMPLE 22

Antitumor Effects of Therapeutic Constructs and Effector T, NKT Cells or Sickled Erythrocytes in Human Patients

All patients treated have histologically confirmed malignant disease including carcinomas, sarcomas, melanomas, lymphomas and leukemia and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4(widespread metastases). Patient history is obtained and physical examination performed along with conventional tests of cardiovascular and pulmonary function and appropriate radiologic procedures. Histopathology is obtained to verify malignant disease.

### EXAMPLE 23

Treatment Procedures

Constructs (or Preparations)

Doses of the constructs are determined as described above using, inter alia, appropriate animal models of tumors. Two classes of therapeutic compositions are administered namely SAg proteins or SAg conjugates (nucleic acids or peptides-polypeptides), SAg phage displays, SAg yeast displays, SAg bacterial cell displays, as described above for animal models.

A treatment consists of injecting the patient with 0.5-500 mg of Construct intravenously in 200 ml of normal saline over a one hour period. Treatments are given 3×/week for a total of 12 treatments. Patients with stable or regressing disease are treated beyond the 12^{th} treatment. Treatment is given on either an outpatient or inpatient basis as needed.

Effector T or NKT Cells

Eligible patients are treated with tumor antigens such as irradiated tumor cells or GM-CSF transduced tumor cells injected approximately 10 centimeters from a draining lymph node site. Ten days post injection, draining lymph nodes are obtained in a limited surgical procedure at the site draining the injection. The lymph nodes are converted to a single cell suspension of lymphocytes and these are incubated with various SAg preparations for two days followed by Il -2 for an additional 72 hours. These lymphocytes now called effector T cells or NKT cell are used for adoptive immunotherapy.

Effector T or NKT cells harvested by centrifugation at 500×g for 15 min and the cell pellets are pooled. After washing the cells in HBSS, the cell are resuspended in 200 ml of normal saline containing 5% human serum albumin and 450,000 IU of IL-2 for transfer. Each recipient will receive four escalating doses or 33 million, 100 million, 330 million and 1 billion cells per square meter of body surface area each given one week apart. Cells are infused through a subclavian central venous catheter over a 30-minute interval. IL-2 administration i.v. is commenced immediately after completion of cell infusion at a dose and schedule of 180,000 IU/ml every 8 h. for 5 days. All patients receive indomethacin (50 mg P.O.) every 8 h, acetaminophen (650 mg P.O.) every 6 h. and ranitidine (150 mg P.O.) every 12 h while receiving IL-2 in order to reduce febrile and gastric side effects. As controls, a cohort of patients is treated with the in vivo tumor vaccination step and IL-2 without the tumor effector cells. Patients will be followed for clinical response every 4 weeks for 2 months with repeat radiological examinations.

Abbreviated Exemplary Human Protocol: Sequential Administration of GM-CSF Transduced Tumor Cells In vivo and SAg Activated NKT and T Cells Ex Vivo in Patients with Metastatic Renal Cell Carcinoma and Melanoma

In Vivo Phase: Immunization with GM-CSF Transduced Tumor Cells

Day 1: GM CSF transfected tumor cells (renal carcinoma/melanoma) are injected as given in Phase I GM-CSF Gene Transduction Protocol [Human Gene Therapy 6: 347-368, (1995)]

Day 7-10: Lymph Nodes draining the GM-CSF transfected tumor cell sites are removed and placed in tissue culture OR patients are pheresed and their peripheral blood T cells and NKT cells collected for further treatment in tissue culture as described below.

Ex Vivo Phase: Immunization with SAg

1. The T cells are obtained from either lymph nodes draining GM-CSF transduced tumor cell immunization or peripheral blood and subdivided into CD4+CD8+ (T cell) and CD4−CD8− (NKT cell) populations.

2. SAg enterotoxin B is added to cultures of the NKT and T cell populations for 48 hours.

3. The NKT cells and T cells are further expanded for an additional 72 hours (optional).

SAg Activated NKT and/or T Cell Administration

1. The CD4+CD8+ (T cell) and CD4−CD8− (NKT) populations are harvested for injection into patients.

2. T cells or NKT cells are administered with a mean 1011 cells per patient.

Assessment:

1. T cells phenotypes for NKT cell markers, V expression, CD44, CD62 are carried out on lymph node and peripheral blood T cells or NKT cells immediately after their removal and at various intervals after ex vivo SAg stimulation and expansion.

2. Tumor and DTH assessment are as described in the Phase I Protocol on GM-CSF Transduction [Human Gene Therapy 6: 347-368 (1995)].

Patient Evaluation

Assessment of response of the tumor to the therapy is made once per week during therapy and 30 days thereafter. Depending on the response to treatment, side effects, and the health status of the patient, treatment is terminated or prolonged from the standard protocol given above. Tumor response criteria are those established by the International Union Against Cancer and are listed in Table VII.

TABLE VII

| RESPONSE | DEFINITION |
|---|---|
| Complete remission (CR) | Disappearance of all evidence of disease |
| Partial remission (PR) | >50% decrease in the product of the two greatest |
| | perpendicular tumor diameters; no new lesions |
| Less than partial remission (<PR) | 25-50% decrease in tumor size, stable for at least |
| | 1 month |
| Stable disease | <25% reduction in tumor size; no progression or new |
| | lesions |
| Progression | >25% increase in size of any one measured lesion or |
| | appearance of new lesions despite stabilization or |
| | remission of disease in other measured sites |

The efficacy of the therapy in a population is evaluated using conventional statistical methods including, for example, the Chi Square test or Fisher's exact test. Long-term changes in and short term changes in measurements can be evaluated separately.

Results

One hundred and fifty patients are treated. The results are summarized in Table VIII. Positive tumor responses are observed in 80% of the patients as follows:

TABLE VIII
All Patients

| Response | No. | % |
|---|---|---|
| PR | 20 | 66% |
| <PR | 10 | 33% |

| Tumor Types | Response | % of Patients |
|---|---|---|
| Breast Adenocarcinoma | PR + <PR | 80% |
| Gastrointestinal Carcinoma | PR + <PR | 75% |
| Lung Carcinoma | PR + <PR | 75% |
| Prostate Carcinoma | PR + <PR | 75% |
| Lymphoma/Leukemia | PR + <PR | 75% |
| Head and Neck Cancer | PR + <PR | 75% |
| Renal and Bladder Cancer | PR + <PR | 75% |
| Melanoma | PR + <PR | 75% |

### EXAMPLE 24

Preparation of DCs

Splenocytes obtained from naive C57BL/6 females are treated with ammonium chloride Tris buffer for 3 min at 37° C. to deplete red blood cells. Splenocytes (3 ml) at 2×10⁷ cells/ml are layered over 2 ml metrizamide gradient column (Nycomed Pharma AS, Oslo, Norway; analytical grade, 14.5 g added to 100 ml PBS, pH 7.0) and centrifuged at 600 g for 10 mm. The DC-enriched fraction from the interface is further enriched by adherence for 90 mm. Adherent cells (mostly DC and a few contaminating macrophages) are retrieved by gentle scraping and subjected to a second round of adherence at 37° C. for 90 min to deplete the contaminating macrophages. Non-adherent cells are pooled as splenic DC, and by FACS® analysis are ˜80-85% DC (stainwith mAb 33D1), 1-2% macrophages (stain with mAb F4/80), 10% T cells, and <5% B cells. The pellet is resuspended and enriched for macrophages by two rounds of adherence at 37° C. for 90 mm each. More than 80% of the adherent population is identified as macrophages by FACS® analysis with 5% lymphocytes and <5% DC. B cells are separated from the non-adherent population (B and T cells) by panning on anti-Ig-coated plates. The separated cell population which is comprised of >80% T lymphocytes by FACS analysis is used as responder T cells

Generation of Bone Marrow-Derived DCs

Erythrocyte depleted mouse bone marrow cells from flushed marrow cavities are cultured in CM with 10 ng/ml GM-CSF and 10 ng/ml IL-4 at 1×10⁶ cells/ml. On day 7, DCs are harvested by gentle pipetting and are enriched by 14.5% (by weight) metrizamide (Sigma) CM gradients. The low density interface containing the DC is collected by gentle a pipette aspiration. The floating DCs express CD11b, CD11c, CD86, DEC205, MHC class I and II and CD40. They are negative or low for CD3 and B220 expression.

DC Cultures

Mouse BM-DCs are prepared in CM with IL-4 and GM-CSF (1000 IU/ml each). The DC arc washed twice with CM, enumerated purity >90% by positive coexpression of MHC class II, CD40, CD80, CD86, and CD11c by fluorescence-activated cell sorter (ACS)], and cultured in CM with added cytokines for further studies. Human-monocyte-derived DCs are obtained from the adherent fraction of mononuclear cells of healthy volunteers and are incubated 7-8 days in AIMV containing L-Glu, antibiotics and rhIL-4 and rhGM-CSF (1000 IU/ml each, Schering Plough, Kenilworth, N.J., USA). After 8 days in culture, the loosely adherent or floating cells show typical dendritic morphology, express high levels of MHC class I and II molecules, CD40 and CD86; most are positive for CD1a and CD11c but low or negative for CD2, CD3, CD14, CD19 and CD83.

### EXAMPLE 25

Preparation of DC/Tumor Cells Hybrids (DC/tc)

DCs derived from BM culture are fused with tumor cells at a 3:1 (DC:tumor cell) ratio using polyethylene glycol (PEG; MW=1450)/DMSO solution (Sigma). In brief, tumor cells are cultured in CM supplemented with 20% FCS and 1×OPI solution (oxaloacetate, pyruvate, and insulin; Sigma) for 4-6 h before fusion. Tumor cells and DCs are then mixed and washed with serum-free medium. After removing the medium, I ml of PEG is added to the cell pellet while resuspending the cells by stirring for 2 min. An additional 10 ml of serum-free medium is added to the cell suspension over the next 3 min. with continued stirring. The cells are centrifuged at 400×g for 5 mm. The cells are resuspended with 20% FCS CM and cultured for 24 h before staining or being used as targets or vaccines. Fusion preparations of DCs with B16 or RMA-S are termed B16/DC and RMA-S/DC, respectively.

Phenotype Staining of Fused Hybrid Cells

B16, RMA-S, DCs, and their fused hybrids are analyzed by staining with FITC- or PE-conjugated mAbs (PharMingen) against MHC antigens (D^{b}, K^{b}, IA^{b}) adhesion and costimulatory molecules (B7.1, ICAM-1) and lymphocyte antigens (Thy-1.2, SmIg) at 4° C. for 45 min. DCs were identified by labeling with mAb against CD11c (N418). B16, B16/DC or B16/B16 fused cells are stained with mAb against AKV Env gp85 protein (M562, provided by Dr. Masaru Taniguchi, Chiba University, Tokyo, Japan) as a B16 tumor-specific marker. RMA-S and RMA-S/DC fused cells are stained with Thy-1.2 or mAb against the R-MuLV-encoded Gag p12 protein (584, provided by Dr. Bruce Chesebro, National Institute of Allergy and Infectious Diseases, Hamilton, Mo.) as RMA-S tumor-derived markers. The method for labeling cells with TRITC (rhodamine) is described. Briefly, cells are resuspended in RPMI 1640 at 1×10⁶ cells/ml and incubated with TR1TC (0.5 g/ml) in 37° C. for 45 mm. The labeled cells are washed three times and used for fusion studies. The phenotypes of fresh and cultured LN T cells is determined by FACS analysis following staining with FITC- or PE-conjugated mAbs against Thy-1.2, Lyt-2, and L3T4 (PharMingen). All cells are washed twice with HBSS and fixed with 0.2% parafornaldehyde. Fluorescence intensity and positive cell percentage were measured on a FACScan flow microfluorometer (Becton Dickinson, Sunnyvale, Calif.).

Additional Fusion Methods

Murine (CS 7BL16) MC38 adenocarcinoma cells are stably transfected with the DF3/MUC1 cDNA (MC38/MUC1). MC38, MC38/MUC1 and the syngeneic MB49 bladder cancer cells are maintained in DMEM supplemented with 10% heat-inactivated fetal calf serum (FCS), 2 mM glutamine, 100 U/ml penicillin and 100 mg/ml streptomycin. DCs are obtained as described from bone marrow culture with certain modifications. Briefly, bone marrow is flushed from long bones, and red cells are lysed with ammonium chloride. Lymphocytes, granulocytes and Ia⁺ cells are depleted from the bone marrow cells by incubation with the following mAbs: (1) 2.43, anti-CD8 (TIB 210; American Type Culture Collection, Rockville, Md.); (2) GK1.5, anti-CD4 (TIB 207); (3) RA3-3A1/6.1, anti B220/CD45R (TIB 146); (4) B21-2, anti-Ia (TIB 229); and (5) RB6-8C5, anti-Gr-1 (PharMingen, San Diego, Calif.) and then rabbit complement. The cells are plated in six-well culture plates in RPMI 1640 medium supplemented with 5% heat-inactivated FCS, 50 M 2-mercaptoethanol, 1 mM HEPES (pH 7.4), 2 mM glutamine, 10 U/ml penicillin, 100 mg/ml streptomycin and 500 U/ml recombinant murine GM-CSF (Boehringer Mannheim, Indianapolis, Ind.). At day 7 of culture, nonadherent and loosely adherent cells are collected and replated in 100-mm petri dishes (10⁶ cells/mil; 8 ml/dish). The nonadherent cells are washed away after 30 mm of incubation, and GM-CSF in RPMI medium is added to the adherent cells. After 18 h, the nonadherent cell population is removed for fusion with MC38/MUC1 or MC38. Fusion is carried out with 50% PEG in Dulbecco's PBS without Ca²⁺ or Mg²⁺ at pH 7.4. The fused cells are plated in 24-well culture plates in the presence of HAT medium (Sigma) for 10-14 days. HAT slows proliferation of MC38/MUC1 and MC38, but not the fused cells. MC38/MUC1 and MC38 cells grow firmly attached to the tissue culture flask, while the fused cells are dislodged by gentle pipetting.

Flow Cytometry

Cells are washed with PBS and incubated with mAb DF3 (anti-MUC1), mAb M1/42/3.9.8 (anti-MHC class I), mAb M5/114 (anti-MHC class II), mAb 16-1OAl (anti-B7-1), mAb GL1 (anti-B7-2) or mAb 3E, (anti-ICAM-1) for 30 mm on ice. After washing with PBS, the appropriate fluorescein isothiocyanate (FITC)-conjugated anti-hamster, -rat and -mouse lgG is added for another 30 mm on ice. Samples are then washed, fixed and analyzed in a FACScan (Becton Dickinson, Mountain View, Calif.).

Fusion of SAg Transfected Tumor Cells with Dendritic Cells

Preparation of Dendritic Cells

Dendritic cells were generated from mouse bone marrow cultures of B6 mouse origin, following the protocol provided by W. Storkus, et a!. Bone marrow cells were prepared from the femurs of four 8 week old normal C576L/6 (denoted B6, H 2^{b} ) mice.

1. For all steps, a complete medium consisting of RPMI 1640 media supplemented with 10% heat-inactivated FCS 0.1 mM NEAA 1 mM sodium pyruvate, 50 mM 2-mercaptoethanol, 50 mM HEPES, 2 mM glutamine, 100 U/ml penicillin, and 100 U/ml streptomycin was used.

2. Following removal of both femurs from each of four mice, cells were extruded by use of a 3 cc syringe filled with complete medium and 25 G needle. Clumps were removed by passing the cell suspension through a Cell Strainer (Falcon 2350). The cell suspension was then centrifuged at 600×g for 5 minutes at room temperature (all further centrifugations were performed in this manner unless otherwise indicated).

3. Red blood cells were lysed by resuspending the cells in 4 ml of Red Blood Cell Lysing Solution (Sigma R7757) and incubating the tube on ice for 2 minutes. After neutralizing the ammonium chloride action with complete medium, the cells were centrifuged to pellet them.

4. Granulocytes and leukocytes were depleted from the bone marrow cells by incubation in 2 ml of a cocktail of monoclonal antibodies from Pharmingen, formulated to contain no azide and have low endotoxin levels. The cocktail consisted of 5 mg/ml of the following monoclonal antibodies (1) 2.43, anti-CDS, Pharmingen 01050D (2) GK 15, anti-CD4, Pharmingen 094200 (3) RA3-3A1/6.1, anti-B220/CD45R, Pharmingen 01120D (4) anti GRI, Pharmingen 01210D. Antibodies were diluted in complete medium, cells were then resuspended in the cocktail and incubated for one hour on ice. After diluting the cell suspension to 30 ml with complete medium, the cells were centrifuged to pellet them.

5. Cells were then resuspended in 12 ml of a 1:8 dilution of low endotoxin rabbit complement (Accurate Chemicals, ACL-3051) which had first been reconstituted in sterile water. The cell suspension was incubated in complement for 30 minutes in a 37° C. water bath. Cells were diluted in complete medium and washed twice by centrifugation before resuspending them in 5 ml of complete medium for counting.

6. The cell concentration was adjusted to 3.3×10⁵ cells/ml in complete medium containing 10 ng/ml IL-4 (Sigma) and 10 ng/ml recombinant murine GM-CSF (Boehringer Mannhaim) and plated at 3 ml/well in 6 well culture plates.

7. Cultures were refed on day 5 by removal and replacement of half the medium. This step was repeated as needed.

8. At day 6 of culture, an aliquot of non-adherent and loosely adherent cells was collected and stained with antibody for N418 antigen (CD11c), a dendritic cell marker, using phycoerythrin (PE) labeled antibody. PE labeled antibody to an irrelevant antigen (TNP) served as the negative control.

9. Cultures were maintained until they were used for fusion with tumor cells.

10. Staining and FACs analysis revealed approximately 75% of the population to be CD11c positive.

Fusion of Dendritic Cells with SEB Transfected B16 F10 Melanoma Cells

One SEB positive clone (clone 11.2) and one vector containing clone (clone 7.5) were maintained in complete medium supplemented with 50 mg/ml G418 and 0.5 mg/ml fungizone.

1. Fusion of dendritic cells with the transtected B16F10 tumor cells, were carried out using preparations from seven and fourteen day cultures of dendritic cells.

2. Fusion of the dendritic cells and tumor cells (either SEB transfected or control vector) was carried out at a 3:1 ratio with PEG (1450 mwt) DMSO (Sigma P7306). Mock fusion consisted of mixing dendritic cells with SEB-transfected cells in the same ratio as used for fusion without PEG-DMSO. In a secpmd experiment, fusion of the dendritic cells and the tumor cells was carried out at a 5:1 ratio.

3. Four to 6 hours before fusion, tumor cells were cultured in complete medium (see dendritic cell preparation) supplemented with 20% FCS and 1×OPI solution (oxaloacetate, pyruvate, and insulin, Sigma). Tumor cells and dendritic cells were mixed and washed three times with serum-free medium, After removing the medium, 1 ml of PEG was added to the cell pellet while resuspending the cells by stirring for 2 min. An additional 10 ml of serum-free medium was added to the cell suspension over the next three minutes with continued stirring. The cells were centrifuged at 400×g for 5 min. and resuspended with complete medium 20% FCS. Cell suspensions were cultured in bulk cultures.

4. In order to eliminate the melanoma cells remaining in the bulk culture, differential adherence subculturing was performed. Cultures were “fractionated” into nonadherent (transfer of the upper half the culture medium with floating cells), loosely adherent (transfer of cells resuspended by gentle pipeting), and adherent (cells requiring trypsinization to recover). Cells transferred to fresh cultures were refed with complete medium containing 20% FCS. Subculturing was performed every 2-3 days or as needed for approximately two weeks.

5. Analysis of dendritic cell-melanoma cell fusions to detect CD11c positive cells was performed on cultures approximately 2 weeks after fusion. Cells were plated at 2×1O⁴ cells/well in four well slide cultures. After three days in cultures, cells were stained with either antibody to N418 antigen (CD11c), a dendritic cell marker, using phycoerythrin (PE) labeled antibody or PE labeled antibody to an irrelevant antigen (TNP) as the negative control. After washing with PBS, the cells were incubated for 30 minutes on ice in PBS with 10% FBS and Fc-Block (Pharmingen 01241D). Five uL/well of the appropriate antibody was added to the wells and the staining was carried out by incubating the slides for 30 minutes on ice. After three washes in PBS, cells were fixed with 2% paraformaldehyde. The remaining cells were frozen for permanent storage.

### EXAMPLE 26

Transfection of Hybrid DC/tc's with SAg DNA or RNA In Vivo and In Vitro

Methods of transfection of SAg-encoding nucleic acid into tumor cell are disclosed in the Examples 1, 32. The same methods are used for transfection of DCs or DC/tc hybrids.

### EXAMPLE 27

Preparation of DCs which have Phagocytosed SAg-Transfected Tumor Cell Lysates or Apoptotic Tumor Cells

PBMCs, DCs, macrophages, and T cells are prepared as follows. In brief, peripheral blood is obtained from normal donors in heparinized syringes and PBMCs are isolated by sedimentation over Ficoll-Hypaque (Amersham Pharmacia Biotech, Piscataway, N.J.). T cell-enriched and T cell-depleted fractions are prepared by resetting with neuraminidase-treated sheep red blood cells. Immature DCs are prepared from the T cell-depleted fraction by culturing cells in the presence of GM-CSF and IL-4 for 7 d. 1,000 U/ml of GM-CSF (Immunex Corp., Seattle, Wash.) and 500-1,000 U/ml of IL-4 (Schering-Plough Corp., Kenilworth, N.J.) are added to the cultures on days 0, 2, and 4. To generate mature DCs, the cultures are transferred to fresh wells on day 7 and MCM is added for an additional 3-4 d. At day 7, >95% of the cells are CD14−,CD83−, HLA-DR^{lo} DCs. On days 10-11, 80-100% of the cells are of the mature CD14−, CD83+, HLA-DR^{hi} phenotype. FACSort® (Becton Dickinson, San Jose, Calif.) is used to generate highly pure populations of immature and mature DCs, based on their CD83− and CD83+phenotypes, respectively. Macrophages are isolated from T cell-depleted fractions by plastic adherence for 1 h. After 24 h, cells are removed from the plates and placed in Teflon beakers for 3-9 d. T cells are further purified from the T cell-enriched fraction by removing contaminating monocytes, NK cells, and B cells.

### EXAMPLE 28

Induction of Apoptotic Death and Phagocytosis of Apoptotic Tumor Cells or SAg-Transfected Tumor Cells by DCs

Monocytes are infected with influenza virus in serum-free RPMI. These cells undergo viral-induced apoptotic death within 6-8 h. Cell death is confirmed using the Early Apoptosis Detection kit (Kayima Biomedical Co., Seattle, Wash.). As previously described, cells are stained with Annexin V-FITC (Ann V) and propidium iodide (P1). Early apoptosis is defined by Ann V+/PI staining as determined by FACScan® (Becton Dickinson). Five to eight h after infection, monocytes first externalize PS on the outer leaflet of their cell membrane, as detected with Ann V. By 8-10 h, these cells are TUNEL (Tdt-mediated dUTP-biotin nick-end labeling) positive. It is not until 24-36 h that the majority of the monocyte population included trypan blue into the cytoplasm, an indicator of secondary necrosis. HeLa cells are triggered to undergo apoptosis using a 60 UV lamp (Derma Control Inc.), calibrated to provide 2 mJ/cm²/s.

Induction and Detection of Apoptosis

Monocytes are infected with influenza virus in serum-free RPMI. Cell death is assayed using the Early Apoptosis Detection kit (Kayima Biomedical). Briefly, cells are stained with Annexin V-FITC (Ann V) and propidium iodide (P1). Early apoptosis is 14 defined by Ann V+/PI− staining as determined by FACScan (Becton Dickinson). Cells from the 293 cell line are triggered to undergo apoptosis using a 60 UVB amp (Derma Control Inc.), calibrated to provide 2 mJcm⁻²s⁻¹.

Phagocytosis of Apoptotic Cells

Monocytes and HeLa cells are dyed red using PKH26-GL (Sigma Biosciences, St. Louis, Mo.), and induced to undergo apoptosis by influenza infection and UV irradiation, respectively. After 6-8 h, allowing time for the cells to undergo apoptosis, they are cocultured with phagocytic cells that were dyed green using PKH67-GL (Sigma Biosciences), at a ratio of 1:1. Macrophages are used 3-6 d after isolation from peripheral blood; immature DCs are used on days 6-7 of culture; and mature DCs are used on days 10-11. Where direct comparison of cells is needed, cells are prepared from the same donor on different days. In blocking experiments, the immature DCs are preincubated in the presence of 50 mg/ml of various mAbs for 30 mm before the establishment of cocultures. After 451 20 mm, FACScan® analysis is performed and double positive cells were enumerated.

Coculture of DCs with Apoptotic Cells

Monocytes from HLA-A2.1-donors are infected with live or heat-inactivated influenza virus. Live influenza virus (Spafas Inc.) is added at a final concentration of 250 HAU ml-1 (MOI of 0.5) for 1 h at 37° C. Virus is heat-inactivated by treatment for 30 min at 56° C. before use. After washing, cells are added to 24-well plates in varying doses. After 1 h, contaminating non-adherent cells are removed and fresh media is added. Following a 10 h incubation at 37° C., 3.3×10³ uninfected DCs and 1×10⁶ T cells are added to the wells.

Antigen Pulsing of DC

Day 7 DC are incubated with freeze-thawed tumor lysates at a ratio of three tumor cell equivalent to one DC (i.e., 3:1) in CM. After 18 hr of incubation, DC are harvested, irradiated with rad (Gamma Cell 1000; Nordion, Kanata, Canada), washed twice in Hank's balanced salt solution (GIBCO), and in Hank's balanced salt solution.

### EXAMPLE 29

Treatment of Tumor Bearing Animals with SAg-Transfected or SAg-Expressing DCs, Accessory Cells or S/D/t Cells: Vaccination Protocols and Treatment of Established Tumor

Immunotherapy

C57BL/6 mice are immunized once with irradiated, S/D/t cells (2×10⁶ cells/mouse) 10-14 d post-immunization mice are challenged with 2×10⁷ live tumor cell subcutaneously in the scapular region. Mice are monitored on a regular basis for tumor growth and size. Mice with tumor sizes >3.5 cm were killed. All survivors were killed 40 d post-challenge.

P10.9-B 16 Melanoma Model

Mice are injected intra-footpad with 2×10⁵ F10.9 cells. Legs are amputated when the local tumor in the footpad is 7-8 mm in diameter. Post-amputation mortality is less than 5%. 2 d post-amputation mice are immunized intraperitoneally with S/D/t cells followed by weekly vaccinations twice, for a total of three vaccinations. Mice are killed based on the metastatic death in the non-immunized or control groups (28-32 d post-amputation). Metastatic loads are assayed by weighing the lungs.

S/D/t cells: In Vivo Immunization and Tumor Challenge

B6 or BALB/c mice are immunized s.c. in the right flank with 1×10⁶ MCA-207 or 1×10⁶ S/D/t cells, respectively, twice at 7-day intervals. Mice then are rechallenged 7 days after the last immunization with a lethal dose of 1×10⁵ MCA-207 (for B6 mice) or 3×10⁵ MT-901 (for BALB/c mice) viable tumor cells by s.c. injections into the left flank. The size of the tumors is assessed in a blinded, coded fashion twice weekly and recorded as tumor area (in square mm) by measuring the largest perpendicular diameters with calipers. Data are reported as the average tumor area SEM (five or more mice per group).

Vaccination Protocol

B6 mice are s.c. immunized twice in a 2-wk interval with 10⁶ irradiated (15,000 rad) B16, B16 mixed with DCs (1/1: unfractionated cells from overnight culture), or S/D/t cells or recombinant formalin fixed bacteria (10⁶-10⁸). Ten days following the final immunization, each group of mice is injected s.c. with varying doses (10⁴, 10⁵, or 10⁶ cells/mouse) of viable B16. Tumor growth and survival time of each group of mice are recorded. The size of the tumor in each mouse is measured in two perpendicular dimensions with a Vernier caliper twice weekly after tumor challenge. Tumor incidence is considered positive when the average diameters of the tumor exceeded 3 mm.

In Vivo Immunization for Treatment of Pulmonary Metastases

B6 or BALB/c mice receive 1.5×10⁵ MCA-207 or 2×10⁵ MT-901 viable tumor cells, respectively, i.v. in the lateral tail vein to establish pulmonary metastases, as described. The mice then are immunized s.c. with, respectively, 1×10⁶ MCA-207 tumor lysate-pulsed S/D/t cells three times on days 3, 7, and 11 or 1×10⁶ MT-901 tumor lysate-pulsed S/D/t cells twice on days 3 and 7 after tumor injection and are killed on days 14 and 17, respectively. Pulmonary metastases are enumerated on day 15 (MCA-207) or 14 (MT-901). Data are reported as the mean number of metastases ±SEM (five or more mice per group).

In Vitro Activation of LN T Cells

B6 mice are immunized s.c. twice in a 2-wk interval on the flanks with 2×10⁶ (10⁶/side) irradiated (15,000 rad) tumor, S/D/t cell preparation, or tumor mixed with DCs (1/1) suspended in 0.1 ml of HBSS. One week after the final immunization, inguinal LNs from each group of mice are harvested. LN cells from each group of mice are activated and expended in culture using anti-CD3 plus IL-2. In brief, LN cells (3-4×10⁶ cells/well) are activated on 24-well plates coated with anti-CD3 mAb (145-2C11) and incubated at 37° C. for 2 days. Alternatively, S/D/t cells (10⁴-10⁵/well) or exosomes (3-5 g) or recombinant bacteria (10⁶-10⁸/well) are incubated with the LN cells for 2 days and optionally with low dose IL-2 for an additional 2 days. The activated cells are suspended at 1-2×10⁵ cells/ml in CM containing IL-2 (4 U/ml) and incubated in gas-permeable culture bags (Baxter Healthcare, Deerfield, Ill.) for an additional 3 days. The derived LN T cells are harvested and used as effector cells for adoptive immunotherapy.

Adoptive Immunotherapy Models

For therapy of B 16 pulmonary metastases. B6 mice are injected i.v. with l05 live B 16 tumor cells in 1 ml of PBS to initiate pulmonary metastases. Three days after tumor inoculation, mice are randomly divided into several groups to receive treatments by i.v. injection of 5×10⁷ cultured LN T cells suspended in 1 ml of PBS. On day 21 after tumor inoculation, mice from each group are killed, and lungs are insufflated with Fekete's solution. Lung metastases are counted. In some experiments. tumor-bearing mice are i.p. administered IL-2 (15,000 U. twice/day for 5 days) following the adoptive transfer of cultured LN T cells. For therapy of FBL-3 tumor. B6 mice are inoculated i.p. with 5×10⁶ viable FBL-3 tumor cells on day 0. By day 5, the tumor is disseminated, and mice are treated with cyclophosphamide (CY) at a dose of 180 mg/kg followed in 6 h by i.p. injection of cultured LN T cells (5×10⁷ cells/mouse) suspended in 0.5 ml of PBS. The tumor growth and the survival time of each group of mice are monitored and recorded on a regular basis

Induction of Anti-Tumor Activity by FC/MUC1.

Groups of 1 mice are immunized twice at 14-day intervals by subcutaneous injection of 3×10⁵ DCs (0) or S/D/t cells represented by FC/MUC1cells. PBS is injected as a control (0). After 14 days, mice are challenged subcutaneously with 2.5×10⁵ MC38/MUC1 cells. Tumors of 3 mm in diameter are scored as positive.

Immunization with FC/MUC1 for Prevention and Treatment of Pulmonary Metastases

Groups of 10 mice are injected twice with S/D/t cells represented by FC/MUC1cells or PBS and then challenged after 14 days with intravenous administration of 1×10⁶ MC38/MUC1 cells. The mice are killed 28 days after challenge. Pulmonary metastases are enumerated after staining the lungs with India ink. Groups of 10 mice are injected intravenously with 1×10⁶ MC38/MUC1 or MC38 cells. The mice are immunized with 1×10⁶ S/D/t cells representing FC/MUC1 cells or FC/MC38 at 4 and 18 days after tumor challenge and then killed after an additional 10 days. Pulmonary metastases are enumerated for each mouse.

Protection Assays

C57BL/6 mice are immunized with the indicated antigen-gene construct. Animals are challenged with tumors and evaluated for tumor survival as described. Briefly, 7 days after the final immunization (day 0), immunized animals are challenged by intradermal injection in the mid-flanks bilaterally with melanoma cells (2×10⁴) at two times the dose lethal to 50% of the animals tested (LD50). Survival is recorded as the percentage of surviving animals. Melanoma cells for injection are washed three times in PBS. Injected cells were greater than 95% viable by trypan blue exclusion. All experiments include five mice per group and were repeated at least three times. Mice that became moribund were killed according to animal care guidelines

### EXAMPLE 30

DNA or RNA from SAg Transfected Tumor Cells, SAg Transfected DCs and SAg Transfected DC/tc Hybrids for In Vivo Vaccination and Transfection of Naive DCs to Produce a DC Expressing SAgs and Tumor Associated Antigens

Plasmid DNA Vector

1. Genes from SAg Transfected Tumor Cells, SAg transfected DCs and S/D/t cells are cloned by PCR to contain a partial or entire coding region. In most cases, it is desirable to not include any sequence 5′ to the ATG or 3′ to the termination codon. PCR primers are designed to contain a restriction site, such as BglII or BamHI.

2. The PCR fragments are separated from unreacted oligomers and template and then the fragment is cut with an excess of BglII for at least 5 h. The DNA is Phenol extracted and ethanol-precipitated. The purified cut fragment is resuspended in TE, pH 8.0 and ligated. to BglII-digested V1J, which has been gel-purified and dephosphorylated with calf intestinal alkaline phosphatase (CLAP), phenol-extracted, ethanol-precipitated, and resuspended in TB, pH 8.0. A 6:1 molar ratio of insert:vector in the ligation reaction is used.

3. Competent E. coli cells (e.g., DH5, DH5a) are transformed with the ligation reaction, plated on L-ampicillin plates and grown overnight at 37° C. Colonies are screened by hybridization of plate lifts to kinase-labeled PCR primer. Several hybridization-positive colonies are selected and grown in overnight cultures for miniprep purification.

4. Miniprep DNAs, are prepared and cut with the appropriate restriction enzymes to determine correct orientation of the gene in the vector. At least three DNAs with the gene in the correct orientation are selected to confirm by sequencing across the ligation junctions. Sequencing primers are designed from the vector sequence. Each primer is 30-50 bp from the restriction site (BglII in the example), so that 10-20 bases within the vector can be read as well as 150-200 bases within the gene. This amount of sequence verifies orientation and give a reasonable estimate of the quality of the PCR-generated gene.

5. DNA preparations that have been sequence-verified 1/1000 in TB, pH 8.0, are diluted and use to retransform competent E. coli. Three isolated colonies from the transformation plates are grown overnight at 37° C., and used to make a −70° C. cell stock by adding 0.8 ml fresh overnight growth to 0.2 ml sterile 80% (v/v) glycerol, mixing well, and freezing on dry ice. The −70° C. stocks are used to isolate plasmid DNA from remaining cells by miniprep procedures. Miniprep DNA is cut again with the appropriate restriction enzymes, and visualized on a gel to verify the construct. All subsequent growth of cells for plasmid production are made from the −70° C. frozen stock. All constructs are tested in vitro to validate their ability to express the desired gene product. Plasmids purified by column (Wizard preps, Promega, Madison, Wis.) or by cesium chloride banding are used to transfect tissue-culture cells transiently. Protein expression is detected by immunoblot. This check not only verifies expression but can validate the size and immunoreactivity of the gene product.

Characterization of Plasmid DNA Vectors

All constructs are tested in vitro to validate their ability to express the desired gene product. Plasmids purified by column (Wizard preps, Promega, Madison. Wis.) or by cesium chloride banding are used to transfect tissue-culture cells transiently. Protein expression is detected by immunoblot. This check not only verifies expression but can validate the size and immunoreactivity of the gene product.

Cell Growth and Transfection

1. DC /tumor cell hybrids, at 0.8-1.5×10⁶ cells/100 mm plate in Dulbecco's Modified Eagle's Medium (DMEM) supplemented with 10% heat-inactivated fetal bovine serum, 20 mM HEPES, 4 mM L-glutamine, and 100 mg/ml each of penicillin and streptomycin, and incubate at 37° C. in 5% CO2 for 18 h.

2. The construct to be tested is cotransfected with 10 mg/plate and 10 g of VIJ-CAT using a calcium phosphate procedure or other methods given in Example 1.

3. Five hours after transfection, the cells are shocked in 15% (v/v) glycerol in PBS, pH 7.2, for 2.5 mm.

4. Cultures are harvested 72 h after transfection by washing the plates twice with 10 ml of cold PBS, pH 7.2, then adding 5 ml of cold TEN buffer and scraping.

5. Pellet cells and use immediately or store at −70° C. for subsequent analysis.

Immunoblot Analysis

1. Cell pellets are lysed in Single Detergent Lysis Buffer, and sonicate on ice (2-15 s bursts) to reduce viscosity.

2. Cell debris is removed by sedimentation and determine soluble protein concentrations of the supernatants by the Bradford method.

3. Equal loadings of soluble cell protein per lane are run on SDS-polyacrylamide gel and transfer the proteins to Immobilon P (Millipore, Bedford, Mass.) membrane.

4. Western blots are incubated overnight with an appropriate dilution of the antibody to the gene product being tested, followed by a 1.5-h reaction with a 1:1000 dilution of peroxidase-conjugated secondary antibody. Develop blots using the ECL kit (Amersham, Arlington Heights, Ill.).

Large-Scale DNA Preparations

1. Expression vectors are grown in E. coli strain DH5 with vigorous aeration in 500 ml growth medium/1-L shake flask. V1J constructs are grown overnight to saturation.

2. Cells are harvested and lysed by a modification of the alkaline SDS procedure. The modification consists of increasing the volumes threefold for cell lysis and DNA extraction.

3. DNA is purified by double banding on CsC1/ethidium bromide gradients.

4. The ethidium bromide is removed by 1-butanol extraction, and the resulting DNA is extracted with phenol/chloroform and precipitated with ethanol.

5. DNA in TE for transfections is resuspended and in 0.9% NaCl for injection into mice.

6. The concentration and purity of each DNA preparation is determined by A 260/280 readings. The 260/280 ratios are >1.8.

7. DNA is stored in small aliquots at −20° C.

### EXAMPLE 31

DNA Immunization In Vivo

1. Animals are housed in an American Association for the Accreditation of Laboratory Animal Care (AAALAC) accredited facility or other national facility and cared for in accordance with the Guide for the Care and Use of Laboratory Animals. Prior to bleeding, or administration of anesthetic or inoculation, animals are in good physical condition and free from stress.

2. For administration of DNA vaccines, animals are anesthetized by ip injection of a solution containing ketamine and xylazine (50 and 20 mg/g body wt, respectively) in a total volume of 0.3 ml of saline. Alternatively, transiently immobilize mice for a sufficient period of time to administer an im injection by allowing inhalation of metophane. Larger animals, such as ferrets or nonhuman primates, are anesthetized using ketamine (30 mg/kg)/xylazine (2 mg/kg)/atropine (1 mg/kg) or ketamine (10 mg/kg), respectively.

3. Fully anesthetized animals are prepared for injection by flooding and swabbing the injection site with ethanol (70%). This provides sterilization and, for small animals, such as mice, facilitates visualization of the muscle groups. To visualize small muscles further, fur around the injection site is shaved followed by ethanol swabbing, or a short incision can be made to permit direct observation of the muscle. In the latter case, the incision is sutured after inoculation.

4. DNA vaccines are administered in saline solution alone or together with a facilitator that induces muscle generation or regeneration. Facilitators are used in animals that may not necessarily be used in humans. For mice, volumes of up to about 50 mL are injected into each quadriceps muscle using a disposable insulin syringe equipped with a 27-gauge needle and having a capacity of 0.3 ml.

5. DNA vaccines are also administered using particle bombardment technology. Plasmid DNA is coated onto gold beads and propelled directly into tissue. Genetic immunization is accomplished by biolistic bombardment using methods similar to those recently described. Briefly, DNA-coated gold particles are prepared by combining 50 mg of 0.95 um gold beads and 100 l of 0.1 M spermidine and sonicating for 5 s. Plasmid DNA (100 mg) and CaCl (200 ml) are added sequentially to the beads spinning in a vortex; mixer. This mixture is allowed to precipitate at room temperature for 5-10 mm. The bead preparation is then centrifuged (10,000 r.p.m. for 30 s) and washed 3 times in cold ethanol before resuspension in 7 ml of ethanol to give a final concentration of 7 mg gold per milliliter. The solution is then loaded into Tefzel tubing (Agracetus, Middleton, Wis.) and allowed to settle for 5 mm. The ethanol is removed and the beads are attached to the side of the tubing by rotation at 20 r.p.m. for 30 s and N2 dried. The dried tubing lined with beads is then cut into 0.5-inch sections and stored for use with desiccant in parafilm-sealed vials. Animals are vaccinated by delivery of two shots (each shot consisted of 0.5 m4j gold beads in 0.5 inch of tubing) to the shaved abdominal region using the Accell gene delivery device (Agracetus) at a discharge pressure of 400 p.s.i. This delivers approximately 1.00 mg/DNA per shot. Animals are immunized with various plasmids In some experiments, particles, are coated with the pGREEN LANTERN-1 plasmid (Gibco BRL, Gaithersburg, Md.), which contains the “humanized” reporter gene encoding GFP from the Aequorecia victoria jellyfish. This gene encodes a naturally fluorescent protein requiring no substrates for visualization.

Formulation of DNA Vaccine:

Saline is the preferred solvent. However, plasmid DNA may also be administered in various other buffer formulations and cationic lipid formulations. Facilitators include anesthetics, such as bupivacaine, and toxins, which are used in conjunction with DNA vaccines. Conventional delivery vehicles are used which facilitate internalization of DNA by cells, protect DNA from digestion by extracellular nucleases, or effect a slow release of DNA; adjuvants are coadministered to provide an additional stimulus for the immune system.

Dosage and Injection Regimen:

DNA vaccines are effective across a broad dosage range. Protective efficacy is achieved with submicrogram amounts of DNA. With respect to humoral immune responses against HA, there is a direct correlation between magnitude of antibody responses and dose of DNA between 10 ng and at least 100 g. However, perhaps owing to viscosity of the solution and/or distribution of the inoculum in the muscle, administration of DNA at concentrations in excess of 2-4 mg/ml results in decreased immunogenicity with some antigens. Therefore, in mice, doses in excess of 200 g are not practical by im injection. The number of injections also directly correlates with magnitude of immune responses (up to at least three). For the influenza model in mice, we have found that three injections given at 3-wk intervals yield optimal protection. It is likely, however, that dosing and regimen will need to be optimized for each gene and challenge model.

Site of Injection:

Injection of plasmid DNA into muscle cells is far superior to other cell types in their capacity to internalize DNA and/or express reporter proteins in vivo. However, immune responses also have been generated after id and iv routes of DNA injection. In addition, particle bombardment of DNA results in the transfection of dermal and epidermal cells leading to the generation of immune responses. The relative effectiveness of these different routes of delivery has yet to be tested rigorously. However, direct im injection generates a protective immune responses at doses (100 ng to 1 g) and is preferred in the range used by particle bombardment.

### EXAMPLE 32

Pulsing DCs with RNA from SAg Producing Bacteria or S/D/t Cells

Total RNA is isolated from SAg producing bacteria or S/D/t cells by standard methods. Pulsing DCs with RNA from SAg producing bacteria, S/D/t cells or SAg transfected tumor cells is performed in serum-free Opti-MEM medium (GIBCO BRL) for tumor extracts with the following modification RNA (25 g in 250 l Opti-MEM medium) and DOTAP (50 g in 250 l Opti-MEM medium) are mixed in 12×75 mm polystyrene tubes at room temperature for 20 mm. The complex is added to the DCs (25×10⁶ cells/ml) and incubated 37° C. in a water bath with occasional agitation for 25 mm. The cells are washed twice and resuspended in PBS (10⁵ RNA pulsed DCs in 500 1 PBS/mouse) for intraperitoneal immunizations. PBS, B16 extract from 10⁵ cells in PBS, or DCs prepared as described above are injected intraperitoneally in a volume of 500 1.

### EXAMPLE 33

PolyA-Cellular RNA from S/D/t Cells or DCs Transfected with SAg:

Preparation and Immunization Protocols

Total RNA is isolated from actively S/D/t cells given above as follows. Briefly, 10⁷ cells are lysed in 1 ml of guanidinium isothiocyanate (CT) buffer (4 M guanidinium isothiocyanate, 25 mM sodium citrate, pH 7.0; 0.5% sarcosyl, 20 mM EDTA, 0.1M 2-mercaptoethanol). Samples are vortexed followed by sequential addition of 100 l 3M sodium acetate, 1 ml water saturated phenol and 200 l chloroform/isoamyl alcohol (49:1). Suspensions are vortexed and placed on ice for 15 mm. The tubes are centrifuged at 10,000 g, 4° C. for 20 min and the supernatant is carefully transferred to a fresh tube. An equal volume of isopropanol is added and the samples are placed at −20° C. for at least 1 h. RNA is pelleted by centrifugation as above. The pellet is resuspended in 300 1 GT buffer which is then transferred to a microcentrifuge tube. RNA is re-precipitated by adding an equal volume of isopropanol and placing the tube at −20° C. for at least 1 h. Tubes are microcentrifuged at high speed at 4° C. for 20 mm. Supernatants are decanted and pellets are washed once with 70% ethanol. Pellets are allowed to dry at RT and then resuspended in TB (10 mM Tris-HCl, 1 mM EDTA, pH 7.4). Possible contaminating DNA is removed by incubating RNA in 10 mM MgCl2, 1 mM DTT and 50 U/ml RNase free DNase (Boehringer-Mannheim, Indianapolis, Ind.) for 15 min at 37° C. The solution is adjusted to 10 mM Tris, 10 mM EDTA, 0.5% SDS and 1 mg/ml Pronase (Boehringer-Mannheim) followed by incubation at 37° C. for 30 mm. Samples are extracted once with phenol-chloroform and once with chloroform, and RNA was then re-precipitated in isopropanol at −20° C. After centrifugation the pellets are washed with 70% ethanol, air dried, and resuspended in sterile water. Total RNA is quantitated by measuring OD at 260 and 280 nm. OD 260/280 ratios are typically 1.65-2.0. RNA is stored at −70° C. PolyA+ RNA is either isolated from total RNA using Oligotex (Qiagen, Chatsworth, Calif.) or directly from tissue culture cells using the Messenger RNA Isolation kit (Stratagene, La Jolla, Calif.) as per manufacturer's protocols.

Production of In Vitro Transcribed RNA The 1.9-kb EcoR1 fragment containing the coding region and 3′ un-translated region is cloned into the EcoR1 site of pGEM4Z (Promega, Madison, Wis.). Clones containing the insert in both the sense and anti-sense orientations are isolated and large scale plasmid preparations are made using Maxi Prep Kits (Qiagen). Plasmids are linearized with BamH1 for use as templates for in vitro transcription. Transcription is carried out at 37° C. for 34 h using the 5P6 MEGAscript In vitro Transcription Kit (Ambion, Austin, Tex.) per manufacturer's protocol and adjusting the GTP concentration to 1.5 mM and including 6 mM m7G(5′)ppp(5′)G cap analogue (Ambion). Template DNA is digested with RNase free DNase I and RNA is recovered by phenol/chloroform and chloroform extraction followed by isopropanol precipitation. RNA is pelleted by microcentrifugation and the pellet is washed once with 70% ethanol. The pellet is air-dried and resuspended in sterile water. RNA is incubated for 30 mm at 30° C. in 20 mM Tris-HCl, pH 7.0, 50 mM KCl, 0.7 mM MnC12, 0.2 mM EDTA, 100 mg/ ml acetylated BSA, 10% glycerol, 1 mM ATP and 5,000 U/ml yeast poly(A) polymerase (United States Biochemical, Cleveland, Ohio). The capped, polyadenylated RNA is recovered by phenol/chloroform and chloroform extraction followed by isopropanol precipitation. RNA is pelleted by microcentrifugation and the pellet is washed once with 70% ethanol. The pellet is air-dried and resuspended in sterile water. RNA is quantitated by measuring OD at 260 and 280 nm and stored at −70° C.

Pulsing of Antigen-Presenting Cells, Accessory Cells DCs, Tumor Cells or DC/Tumor Cell Hybrids with RNA Derived from S/D/t Cells

Pulsing of cells with RNA is routinely performed in serum-free Opti-MEM medium (GIBCO BRL). Cells are washed twice in Opti-MEM medium. Cells are resuspended in Opti-MEM medium at 25×10⁶ cells/nil and added to 15 ml polypropylene tubes (Falcon). The cationic lipid, DOTAP, (Boehringer Mannheim) is used to deliver RNA into cells. RNA (in 250-500 1 Opti-MEM medium) and DOTAP (in 250-500 p.1 Opti-MEM medium) are mixed in 12×75-mm polystyrene tubes at room temperature (RT) for 20 mm. The amount of polyA+RNA or IVT RNA used is 5 g and the amount of total RNA used is 25 g. The RNA to DOTAP ratio is 1:2. The complex is added to the APC (2-5×10⁶ cells) in a total volume of2 ml and incubated at 37° C. in a water-bath with occasional agitation for 2-4 h.

### Example 34

In vivo Immunization with RNA Derived from “S/D/t Cells” or SAg-Transfected Tumor Cells

Preparation of mRNA for Transfection

DNA is linearized downstream of the poly A tail with a 5-fold excess of PstI. The linearized DNA is then purified with two phenol/chloroform extractions, followed by two chloroform extractions. DNA is then precipitated with NaOAc (0.3M) and 2 volumes of EtOH. The pellet is resuspended at about 1 mg/ml in DEP-treated deionized water. A transcription buffer is prepared, comprising 400 mM Tris. HCl (pH 8.0), 80 mM MgCl2, 50 mM DTT, and 40 mM spermidine. The following materials are added in order to one volume of DEP-treated water at room temperature: 1 volume T7 transcription buffer; rATP, rCTP, and rUTP to 1 mM concentration; rGTP to 0.5 mM concentration; 7 g(5′)ppp(5′)G cap analog (New England Biolabs, Beverly, Mass.) to 0.5 mM concentration; the linearized DNA template to 0.5 mg/ml concentration; RNAsin (Promega, Madison, Wis.) to 2000 U/ml concentration; and T7 RNA polymerase (N.E. Biolabs) to 4000 U/ml concentration.This mixture is incubated for 1 hour at 37° C. The successful transcription reaction is indicated by increasing cloudiness of the reaction mixture.

Following generation of the mRNA, 2U RQ1 DNAse (Promega) per microgram of DNA template used is added and was permitted to digest the template for 15 minutes. Then, the RNA is extracted twice with chloroform/phenol and twice with chloroform. The supernatant is precipitated with 0.3M NaOAc in 2 volumes of EtOH, and the pellet isresuspended in 100 mu 1 DEP-treated deionized water per 500 1 transcription product. This solution is passed over an RNAse-free Sephadex G50 column (Boehringer Mannheim #100 411) . The resultant mRNA is sufficiently pure to be used in transfection of vertebrates in vivo.

mRNA Vaccination In Vivo

A liposomal formulation containing mRNA coding for the SAg/tumor associated antigen protein prepared and is inserted into the plasmid pXBG in A volume of 200 1 of a formulation is prepared containing 200 mg/ml of S/D/t cell-derived mRNA and 500 mg/ml 1:1 DOTAP/PE in 10% sucrose is injected into the tail vein of mice 3 times in one day. At about 12 to 14 h after the last injection, a segment of muscle is removed from the injection site, and prepared as a cell lysate according to Example 7. The S/D/t cell-derived specific protein is identified in the lysate.

Severe combined immunodeficient (SCID) mice (Molecular Biology Institute, (MBI), La Jolla, Calif.) were reconstituted with adult human peripheral blood lymphocytes by injection into the peritoneal cavity according to the method of Mosier (Mosier et al., Nature 335:256 (1988)). The mice were maintained in a P3 level animal containment facility in sealed glove boxes. mRNA coding for the S/D/t cell-derived proteins is prepared by obtaining the S/D/t cell gene in the form of a plasmid removing the gene from the plasmid; inserting the gene into the pXBG plasmid for transcription; and purifying the transcription product S/D/t cell-derived mRNA. The S/D/t cells mRNA is then incorporated into a formulation and 200 1 tail vein injections of a 10% sucrose solution containing 200 mg/ml S/D/t cell RNA and 500 mg/ml 1:1 DOTAP:DOPE (in RNA/liposome complex form) were performed daily on experimental animals, while control animals were likewise injected with RNA/liposome complexes containing 200 mg/ml yeast tRNA and 500 mg/ml 1:1 DOTAP/DOPE liposomes. At 2, 4 and 8 weeks post injection, biopsy specimens are obtained from injected lymphoid organs and prepared for immunohistochemistry.

A volume of 200 1 of the formulation, containing 200 mg/ml mRNA from S/D/t cells, and 500 mg/ml 1:1 DOTAP:DOPE in 10% sucrose is injected into the tail vein of the human stem cell-containing SCID mice 3 times in one day. Following immunization, the mice are challenged by tumor inoculation.

The full-length sequence for the cDNA of the S/D/t-derived gene is obtained and ligated to BgIII linkers and then digested with BgIII. The modified fragment is inserted into the BgIII site of pXBG. S/D/t-derived protein is transcribed and purified mRNA is incorporated into a formulation. Balb 3T3 mice are injected directly in the tail vein with 200 1 of this formulation, containing 200 mg/ml of S/D/t-derived mRNA, and 500 mg/ml DOTAP in 10% sucrose.

### Example 35

Preparation of “String of Beads” Tumor Antigens for Transfection of SAg-Transfected DCs, Other Accessory Cells or Tumor Cells

Generation of rAd

All cell lines were maintained in Iscove's modified Dulbecco's medium (IMDM) (Scromed, Berlin) supplemented with 4% fetal calf serum (FlyClone), penicillin (110 international units/ml; Brocades Pharma, Leiderdorp, The Netherlands). and 2-mercaptoethanol (20 mM) at 37° C. in a 5% CO2 atmosphere. The adenoviral vector construction adapter plasmid pMad5 is derived from plasmid pMLPI0 as follows. pMLPI0-lin is constructed by insertion of a synthetic DNA fragment with unique sites for the restriction endonucleases MluI, SplI, SnaBI, Spel, Asull, and MunI into the Hindlll site of pMLP10. Subsequently, the adenovirus BglII fragment spanning nucleotides 3328 8914 of the AdS genome is inserted into the Mun1 site of pMLP-lin. Finally, the Sal1-BamHI fragment is deleted to inactivate the tetracycline resistance gene, resulting in plasmid pMad5. A mini-gene cassette vector, pMad5-0. is generated by ligation of the annealed and phosphorylated double-stranded oligonucleotides 1a/b and 2a/b into the Mlul and Spel sites of pMad5. This cloning step leads to elimination of the original MluI and SpeI sites and to creation of a small ORF, which essentially consists of a start codon, the sequence SEOKLISEEDLNN, a human c-Myc-derived sequence, which is recognized by mAb 9E10 and a stop codon. A small “stuffer” sequence. flanked by newly generated MluI and SpeI sites, is present between the start codon and the c-Myc sequence.

pMad5-1 and -2, each of which harbor a multi-epitope encoding minigene. are constructed by unidirectional cloning of the following double-stranded oligonucleotides into pMad5-0, which had been cleaved with MluI and SpeI. pMad5-I. After each cloning step, the sequence of the inserts is verified by DNA sequencing. Expression of these minigenes is driven by the Ad5 major late promoter, which in this configuration is linked to the AdS immediate early enhancer, resulting in immediate early expression of the minigenes. rAds are generated through in vivo homologous recombination in the Ad5E1-transformed helper cell line 911 between plasmid pJMI7. containing the sequence of the AdS mutant d1309, and either of the plasmids pMad5-1 or pMad5-2. 911 cells are transfected with 10 g of plasmid pJMI7 in combination with 10 g of either pMad5-1 or pMad5-2. The rAds are plaque-purified three times, after which the clonal rAds are propagated in 911 cells, purified by double cesium chloride density gradient centrifugation. and extensively dialyzed. The presence of replication-competent adenoviruses is routinely checked by infection of Hep-G2 cells. The viral stocks were stored in aliquots with 10% glycerol at −80° C. and titered by plaque assay using 911 cells.

Further Transfection of SAg-Transfected DCs, Accessory Cells or Tumor Cells

In short, 100 ng of plasmid DNA encoding Ad5LI, HPV 16 E7, murine p53 or the influenza-matrix protein are transfected into 1×10⁴ SAg-transfected DCs, accessory cells or tumor cells. The transfected cells are incubated in 100 ml of IMDM containing 8% fetal calf serum for 48 h at 37° C., after which 1500-500 CTL ??? in 25 ml of IMDM containing 50 Cetus units (=300 international units) of recombinant interleukin-2 (Cetus) are added. After 24 h, the supernatant is collected, and its tumor necrosis factor (TNF) content is determined by measuring its cytotoxic effect on WEHI-164 clone 13 cells.

### Example 36

Production of Exosomes from DCs Expressing SAg and Tumor Associated Antigens and Normal Hepatocytes.

Exosome Isolation

SAgs or tumor associated antigens are transfected into tumor cells, DCs, or DC/tc hybrids by methods disclosed herein.. The SAg-encoding nucleic acid is provided with sorting sequences which route the translated protein to the endoplasmic reticulum and thereupon to secretory vesicles or exosomes. Alternatively, tumor cells, DCs or DC/tc are incubated 18-20 hours with tumor peptides or SAgs. DCs supernatants are harvested, centrifuged (at 4° C.) at 300 g for 20 mm and then at 10,000 g for 30 min (to eliminate cell debris). Exosomes are then pelleted at 100,000 g for one hour, and washed once in a large volume of PBS (over 100-fold the final volume of resuspension of the exosomes). The protein concentrations in exosome preparations is measured by Bradford assay (BioRad). The slightly acidic pH transiently induced by the acid peptide elution increases the amounts of exosomes produced by DCs. Three to five g of exosomes are routinely obtained from 5-10×10⁵ DCs in 18-20 hours. Exosomes containing LDL, oxyLDL, apolipoproteins, LDL receptors and oxyLDL receptors are obtained from normal hepatocytes by a method similar to that described above for dendritic cells and sickled erythrocytes as in Example 6.

Mice and Tumor Cell Lines for Exosome Trials

DBA/2J (H-2^{d}) and BALB/c (H-2^{d}) female mice 6-8 weeks of age are raised in pathogen-free conditions. P815 (H-2^{d}) is a methylcholanthrene induced mastocytoma, syngeneic with DBA/2. TS/A (H-2^{d}) is a spontaneously-arising undifferentiated mammary adenocarcinoma, syngeneic with BALB/c. All tumor cell lines are maintained in RPMI 1 640 supplemented with 10% endotoxin-free fetal calf serum (Gibco BRL), 2mM L-Glutamine, 100 U/ml penicillin, 100 mg/ml streptomycin, essential amino acids and pyruvate.

Experimental Mouse Models for Exosome Trials

Twice the minimal tumorigenic dose of tumor cells (5×10⁵ P815, 10⁵ TS/A) is inoculated intradermally in the upper right flank of DBA/2 and BALB/c mice, respectively. Animals with established tumors at days 3-4 for TS/A, or days 8-10 for P815, are immunized with a single intradermal injection of 3-5 g of exosomes per mouse in the lower ipsilateral flank. The tumor size is monitored biweekly and mice are sacrificed when bearing ulcerated or huge tumor burdens. All experiments are performed two to three times using individual treatment groups of five mice per group.

### Example 37

Bacterial Constructs for the Expression of SAgs Linked to Galactosylceramides, a-Gal Epitope, Peptidoglycans, Lipopolysaccharides and b1,3-Glucans

Nucleic acids encoding SAgs may be transfected into bacteria which naturally synthesize and express fundamental recognition units for innate immunity. Some of these moieties such as monogalactosylceramides and a-galactosylceramides are potent immunogens and induce anti-tumor activity. The addition of the SAg and a dominant tumor associated epitope coexpressed with these natural bacterial constructs and administered to a tumor bearing host would promote a potent tumor specific response. The system described uses S. carnosus as a model bacterial system to express a SAg peptide and dominant tumor epitope.

Expression Vectors for Surface Display

The shuttle vector constructed pSPPmABPXM consists of the following parts: (i) the origin of replication for E. coli and the 13-lactamase gene giving ampicillin resistance for transformed E. coli cells; (ii) the origin of replication for phage f1; (iii) the origin of replication from S. aureus and the chloramphenicol acetyltransferase gene for staphylococcus expression; (iv) the promoter, signal sequence, and propeptide sequences from the S. hyicus lipase gene construct, optimized for expression in S. carnosus; (v) a multicloning site containing three unique recognition sites for restriction endonucleases; (vi) a gene fragment encoding a serum ABP from streptococcal protein G; and (vii) gene fragments encoding the cell wall-anchoring regions X and M from staphylococcal protein A.

As a model system, the surface display of SAg staphylococcal enterotoxin B. substitutes for place the 80 amino acid malaria peptide M3 from falciparum blood stage antigen Pf155/RESA.. A plasmid vector, pSPPM3ABPXM is constructed, in which a gene fragment encoding SEB instead of M3 is introduced between the propeptide region and the ABP sequence of plasmid pSPPmABPXM. An oligonucleotide linker (5′AGCTTGGCTGTTCCGCCATGGCTCGAG-3′ with complementary sequence) is inserted into the HindIII site of plasmid pSZZmpISX thus creating additional NcoI and XhoI recognition sites downstream of the HindIII site in the resulting vector, pSZZmpI8XhoXM. A gene fragment encoding a 198-amino-acid ABP from the serum albumin binding region of streptococcal protein G is generated by a PCR (primers 5′-CCGAATTCAAGCTTAGATGCTCTAGCAAAAGCCAAG-3′ and 5′-CCCCTGCAGTTAGGATCCCTCGAGAGGTAAAATTTCATC-3′ respectively) with plasmid pSPGI as template sequenced in plasmid pRIT28 by solid-phase DNA sequencing and HindIII-XhoI subcloned in frame downstream of the mpl8 multilinker of pSZZmp18XhoXM. yielding plasmid pSZZmpI8ABPXM. An M3-encoding gene fragment was BamHI-HindIII subcloned from plasmid pRIT28EM3DAStop into pSZZmpI8ABPXM, yielding plasmid pSZZM3ABPXM. Plasmid pLipPS17 is constructed from pLipPSlk the introduction of a BsmI recognition site in the beginning of the lipase signal sequence. a Bc/I site at the end of the signal sequence and a BglII site at the end of the propeptide-encoding region by site-directed in vitro mutagenesis. A gene fragment constituting almost the entire S. carnosus vector pLipPSI except for a fragment encoding the C terminus of the propeptide and the majority of the mature lipase from S. hyicus is isolated by SalI-HindIII digestion and ligated to the E. Co/i plasmid pRIT28. which had previously been cut with the same restriction endonucleases. The resulting plasmid. designated pSDLip. contained the origin of replication for both E. coli and S. aureus. To restore the C-terminal region of the lipase propeptide, a gene fragment encoding the C-terminal part is generated by PCR amplification with the oligonucleotides 5-CCGAATTCTCGAGGCTCCTAAAGAAAATAC-3′ and 5′-CCAAGCTTGGATCCTGCGCAGATCTTGGTGTTGGTTTTTTG-3′ as upstream and downstream primers. respectively, with plasmid pLipPS17 as template. This amplification introduced upstream EcoRI and XhoI sites and downstream FspI. BamHI. and HindIII recognition sequences by noncomplementary sequences in the PCR primers. The gene fragment encoding the C-terminal propeptide region was EcoRI-HindIII subcloned to pRIT28 to verify a correct sequence by solid-phase DNA sequencing and thereafter XhoI-BamHI transferred to SalI-BamHI-restricted pSDLip.

The resulting plasmid. pSPP is HindIII restricted, filled in with Klenow polymerase. and religated to yield plasmid pSPPDHind. which encodes the signal peptide and the complete propeptide of the S. hyicus lipase with transcription from a promoter region suitable for overproduction in S. carnosus.

### Example 38

Gene Transfer for Expression of an Mono or Digalactosylceramide by Transfection with a Cosmid Genomic Library Prepared from a Cell Line in which the Specific Glycosylceramide is Highly Expressed

The deliberate transfer of mono or digalactosylceramide expression in tumor cells is achieved by transfection with a cosmid DNA library prepared from Fabry's cells in which the mono or digalactosylceramide is highly expressed. This model demonstrates a general method for transferring glycosyltransferase genes and other factors necessary for the expression of glycosphingolipid antigens. The recipient tumor cells contain mono or digalactosylceramide and the direct precursor, lactosylceramide. The transfected cells express mono or digalactosylceramide detected both chemically and immunologically and contained human DNA detected by an Alti sequence probe.

Cells and antibodies: Fabry's cells or normal cells with an a-galactosidase deficiency and tumor cells including but not limited to neuroblastoma cells are used. Anti-galactosyl ceramide monoclonal antibody is prepared. Total DNA is prepared from Fabry's cells is excised by MboI and ligated by Bam HI-treated cosmid vector PCV 108, which has the SV40 promotor fused to the neomycin phosphotransferase gene. The target DNA for cosmid cloning is purified by gel electrophoresis between 30-40 KB size. In vitro packaging is made with an extract of lysogenic bacteria and propagated in E. coli. as described elsewhere.

Transfection and Selection of Galactosylceramide Expression: Cosmid library DNAs are transfected into various cells using the calcium phosphate DNA precipitation technique with the addition of a glycerol shock after a 6 hour incubation. Galactosylceramide selection is started 2 days later at 400 mg/ml concentration. The expression of galactosylceramide in the original Fabry's cell and the transfected tumor cells was determined by cytofluorometry (FACS II), in which FITC-conjugated anti-mono or digalactosylceramide antibody is used. Glycolipids in transfected cells are analyzed after cells were extracted in chloroform-methanol (2:1 and 1:1 v/v). The neutral glycolipid fraction is prepared by an acetylation procedure. The glycolipid profile is confirmed on HPTLC, followed by immunostaining with anti- mono or digalactosylceramide antibody.

### Example 39

Staphylococcal Collagen Binding Adhesin Nucleic Acids Transfected into SAg Transfected Tumor Cells, SAg Transfected DCs or Accessory Cells and S/D/t Cells

Collagen gene fragments from S. aureus strain FDA 574 are overexpressed in E. coli using the vector pQE-30 (QIAGEN inc. Chatworth, Calif.). Recombinant proteins expressed from this vector contain an NH2-terminal tail of six histidine residues. The gene named cna encoding a S.aureus collagen adhesin is isolated from a S. aureus genomic library cloned and sequenced. The cna gene encodes a 1185 amino acid polypeptide. The deduced amino acid sequence reveals several structural characteristics similar to previously described Gram-positive bacterial cell surface proteins.

Plasmids expressing cna gene fragments are produced as follows. Recombinant S. aureus collagen adhesin fragments are overexpressed in E. coli using three different prokaryotic expression systems. The amino terminus including the entire A domain is amplified from S. sureus FDA 574 chromosomal DNA using PCR together with primers CNA 20 and CNA 21. The amplified 1.6-kb cna gene fragment is cleaved with EcoRI and PstI, gel purified and ligated to the prokaryotic expression vector pKK223-3 obtained from Pharmacia LKB Biotechnology to create plasmid pKK1.5. Expression vector pKK223-3 contains an IPTG-inducible tac promoter adjacent to a consnesus Shine-Dalgarno ribosomal binding site. However, this vector lacks an initiation codon; therefore, the DNA to be expressed must contain an appropriate start codon. In order to express an internal cna fragment, a DNA linker sequence containing an ATG start codon is synthesized. Two partially complementary ologonucletides, JPI (5′AATTACCATGGAATTCCTGCA-3′) and JP2 (5′-TGGTACCTTAAGG-3′), are heated to 70° C. and slowly cooled to allow annealing. Once annealed, the double-stranded linker is phosphorylated by the addition of ATP and T4 polynucleotide kinase. The DNA linker contained EcoRI and PstI restriction sites at the 5′- and 3′-termini, respectively. These sites are used to insert the linker onto pKK223-3. A 2.9-kb EcoRI/PstI DNA fragment, originally isolated from lambdaGT11 clone pCOL11 was ligated to vector pKK223-3 to create plasmid pKK2.9. The collagen adhesin fragment encoded by pKK2.9 contains three repeated domains (B1, B2, and B3), the carboxyl terminus and downstream sequences.

The plamid containing the collagen adhesin is transfected into DTES by methods in Example 1 and 3 and expression of the transduced gene is monitored by Immunoblots (Example 33).

### Example 40

Transfection of Nucleic Acids Encoding SAgs in Combination with Nucleic Acids the Promote Apoptosis Induction or Predispose to Apoptosis

SAgs expressed in apoptotic tumor cells or tumor cell/DC hybrids are ingested by DCs which present them to the immune system in more which evokes a potent immune response to the tumor associated antigens. The apoptotic cell is also one which is overexpresses a GalCer such as one with a natural or acquired a-galactosidase deficiency or from a patient with Fabry's Disease. The apoptotic stimulus can be produced by concordant influenzal infection, radiation or chemotherapy. In addition, it may be inducible by an exogenous source such as TNF if the cell is predisposed by transfection * of an potent inhibitor of NF-kB such as a modified form of IkBa. Additional stimuli to apoptosis are provided by numerous well established activators (caspase 9) or initiators (caspase 8) of the caspase system or the CD95 TNFR network. Having undergone apoptosis, the SAg transfected, GalCer overproducing cell is now ingested by DCs which are cross-primed to present the tumor antigens and the GalCer in the context of SAg stimulation resulting in a potent antitumor response. Methods and protocols for SAg transfection are given in Example 1 and for priming of DCs in Example 27-28 The apoptotic transfectants are used as a preventative or therapeutic antitumor vaccine by protocols in Example 15, 16, 18-23 and 29. They are also useful ex vivo to a population of tumor specific effector T cell or NKT cells for use in the adoptive immunotherapy of cancer (Examples 2-5, 7, 15, 16, 18-23, 29).

### Example 41

Preparation and Isolation of Glycosphingolipids and Verotoxins Galabiosylceramide, Globotrioslceramides and Globotetraosylceramide

Globotrioslceramides (GB3) and globotetraosylceramide (Gb4) are purified from human renal tissue. Briefly, the chloroform/methanol tissue extract is first applied on a Bio-Sil A (Bio-Rad) silica column in chloroform. The column is extensively washed with chloroform, and neutral glycolipids are eluted with acetone/methanol,9:1 (vol/vol). The neutral glycolipid fraction is then applied on a second Bio-Sil A column in chloroform/methanol, 98:2 (vol/vol). Glycolipids are then resolved with a linear solvent gradient comprising equal weights of chloroform/methanol 15:1 (vol/vol), to chloroform/methanol, 4:1 (vol/vol). Galabiosylceramide (Gb2) or Gal(α1-4)Gal ceramide from marine sponge may be obtained, for example, from Dr T. Matsubara (Department of Chemistry, Kinki University, Kowakae. Japan).

VTs and Subunits

A simple method for purifying E. coli H30 verocytotoxin is as follows. The toxin, released from the cells by exposure to polymyxin B, is subjected to differential ammonium sulfate precipitation and sequential chromatography on hydroxylapatite, chromatofocussing, Cibachron blue, and Sephadex G-100 columns. The purified toxin, 39 kDa by gel filtration and having a pI of 6.72, resolves as a band which migrates at 32 kDa and another band of less than 14 kDa which migrates with the buffer front on reducing SDS-PAGE. The purified preparation is relatively heat-stable, and has a specific activity of 3×10⁹ CD50 units/mg protein in Vero sells, and LD50 values of 0.2, 9.0, and 40 g protein/kg in rabbits, rats, and mice, respectively. Antiserum to the toxin specifically neutralizes H 30 VT, Shiga toxin, and VT activity from some clinical isolates of VT⁺ E. coli but not that from a porcine edema disease strain.

Verocytotoxin 2 (VT2) is purified from E. coli strain E32511 using, as starting material, cells harvested from a Penassay broth culture incubated for 6 h at 37° C. in the presence of mitomycin C (0.2 mg /mil). A crude extract of VT2, is obtained by polymyxin B treatment of cell pellets, is purified using differential ammonium sulphate precipitation, and sequential column chromatography. The purified toxin is estimated to have a pI of 6.5 by chromatofocusing and a molecular weight of 42 kDa by gel filtration; it has a specific activity of 1.39×10⁶ CD50 units/mg protein in Vero cells, and resolves as a major band of Mr 35 kDa and another band of <14 kDa which migrates with the buffer front on reducing SDS-PAGE. The purified toxin is not neutralized by VT1 antisera, and antisera prepared to this toxin in rabbits did not neutralize VT1, but completely neutralized the activity of the homologous toxin.

Recombinant Methods of Preparing VT's and Subunits

Recombinant VT1 is purified from pJLB28. VT2 from R82. and VT2c from E32511. The recombinant E. coli strain pJLB28 is used as a source of VT1 B subunit. High yields of the toxins or subunits (10-15 mg/3 liters of broth culture) are purified by a method involving polymyxin B extraction, ultrafiltration, hydroxylapatite chromatography, chromatofocusing, and Cibacron Blue chromatography. VT2 is purified by virtually the same method from an E. coli clinical isolate, strain E32511. The cistron encoding the B subunit of E. coli Shiga-like toxin I (SLT-I) is cloned under control of the tac promoter in the expression vector pKK223-3 and the SLT-I B subunit is expressed constitutively in a wild-type background and inducibly in a lacI^{q} background. E. coli TB 1 lac pro rpsL ara thi f80d LacZ D M15 hsdR is obtained from Bethesda Research Laboratories (Gaithersburg, Md.). E. coli JMlO1 D lacc pro supE thi (F′ traD36 IacZ A MIS pro AB IacP) is obtained from Dr. J. D. Friesen (Department of Medical Genetics, University of Toronto, Toronto, Ontario, Canada). Plasmids pTZ18R and pKK223-3 are obtained from Pharmacia. Plasmid pJLB5 consists of a 3.0 kb KpnI fragment of bacteriophage H 19B DNA cloned in the KpnI site of pUC18. To construct plasmid pJLB34, pJLB5 is cut at the BglII site and digested with nuclease Bal31. The ends are filled with Klenow fragment and dNTPs. The fragment remaining after deletion is cleaved with EcoRI, and the piece carrying the SLT-I B cistron is purified by agarose-gel electrophoresis. The fragment is recovered from the gel and cloned into pUC18 cut with EcoRI and HindII. The EcoR1-HindIII fragment is cloned in M13mp18 and its nucleotide sequence is determined. The B cistron coding sequence is recovered from pJLB34 as a 1.1 kb Pst1 fragment and was then cloned in the PstI fragment and was the cloned in the PstI site of the polylinker of pKK223-3. Clones with the correct orientation of insertion relative to the tac promoter are identified by restriction-endonuclease analysis. One plasmid with the orientation is selected and designated pJLB120. pJLB120 is transformed into E. coli TB1 for constitutive expression and into E. coli JM101 for inducible expression. Bacteria are grown in L-broth or brain heart infusion broth (Difco Laboratories, Detroit, Mich.) supplemented as necessary with carbenicillin at 50 mg/ml and IPTG (Bethesda Research Laboratories) at 1 mM.

Expression of Toxins

For E. coli JM 101 (pJLB 120), an overnight culture is used to inoculate fresh L-broth supplemented with carbenicillin (50 pg/ml) and was grown to mid-exponential phase (A600=0.3-0.6) at 37° C. with shaking at 300 rev./min. IPTG is added to a final concentration of 1 mM. and incubation is continued with aeration. For E. coli TB I (pJLB120), an overnight culture is used to inoculate fresh L-broth supplemented with carbenicillin (50 mg/ml), and this is grown for 12-18 h at 37° C., with shaking at 300 rev./min. In both cases the culture is harvested and the pellet is washed once with PBS (0.15M-NaCl/10 mM sodium phosphate buffer, pH 7.4) before extraction.

Polymyxin B Extraction of Toxins

The washed pellet is resuspended in PBS containing 0.1 mg/ml polymyxin B in one-quarter of the original culture volume and extracted as previously described. For purification, 18 h cultures of E. Coli TBI (pJLB 120) are extracted with polymyxin B, and the extracts are concentrated 10-fold using a stirred-cell Amicon concentrator with a Ym-5 membrane (Amicon Corp., Danvers, Mass., USA).

Quantification of Toxins

Periplasmic extracts of VT-producing clones, prepared by polymyxin B extraction, are diluted as required and filtered onto nitrocellulose paper in a slot-blot apparatus (Bio-Rad Laboratories). VT is detected by using MAb 1 3C4 according to the Western-blot procedure described above. Blots are scanned with a Molecular Dynamics model 300A computing densitometer. VT is quantified by comparison with a standard curve generated with purified B subunit protein.

Purification of Toxins

The concentrated polymyxin B extract are dialysed overnight against 50 mM-Tris/HCl buffer, pH 7.4, and then applied to a DEAE-Sephacel column (1 cm×20 cm) equilibrated with 1 mM-Tris/HCL buffer, pH 7.4. Bound material is eluted by using a linear gradient of 0-1M-NaCl in 50 mM-Tris/HCl buffer, pH 7.4, and 5 ml fractions are collected. Fractions containing VT are identified, pooled and concentrated with Centriprep-3 concentrators (Amicon Corp.). This pool is dialyzed overnight against 25 mM-imidazole/HCl buffer, pH 7.4, and is applied to a column (1.5 cm×20cm) of Polybuffer exchangcr 94 (Pharmacia) equilibrated with the same buffer. Elution is carried out with a degassed solution of Polybuffer 74 (Pharmacia) diluted 1:8 with distilled water and adjusted to pH 4.0 with HCl (11column volumes). Fractions (5 ml) are collected, and the B subunit positive fractions are pooled and concentrated with Centriprep-3 (Amicon). Ampholytes are removed by Sephadex G-50 gel-filtration.

HPLC Purification of Toxins

Approximately 1 mg (in 1 ml) of purified toxin or subunit is injected into a TSK-G2000SW HPLC gel filtration column previously equilibrated with 50 mM Tris-buffered saline (TBS), pH 7.4, flow rate of 1.0 ml/mm. Peaks, measured by absorbance at 1=280 nm, arecollected.

Toxin Subunit Separation

1 mg of toxin subunit is concentrated to 30-50 ml using a Centricon 30 concentrator (Amicon). 1 ml of a subunit dissociating solution (6 M urea, 0.1 M NaCl, 0.1 M propionic acid, pH 4, is added dropwise, and the toxin is incubated without stirring at 4° C. for 1 h. The solution is then separated by HPLC gel filtration (as above) after previous column equilibration with the dissociating solution. Peaks, measured by absorbance at 1=280 nm, are collected.

### Example 42

Gangliosides Shed from Tumor Cells: Isolation from Tumor Cell Supernatants Collection of Tumor Cell Supernatant

Tumor cells are cultured in 25 ml of no serum-low protein medium (NSLP) in an 80 cm² flask for 1-5 days. Cells are harvested by centrifugation at 400 g for 10 mm, and the supernatant is concentrated 10-fold at 4° C. in an Amicon stirred cell with a 10-kDa cutoff ultrafilter. Concentrated supernatant and NSLP concentrated under the same conditions are stored at −20° C., and passed through a 0.1 -um sterile membrane filter.

Metabolic Labeling of Gangliosides in Tumor Cell Supernatant

Tumor cells (1×10⁵/ml) are cultured in 10 ml of NSLP for 2 days. After three washes with fresh medium, cells are transferred into 10 ml of NSLP containing 1 mCi/ml D-[1-¹⁴C]GlcNH2-HCl (50 mCi/mmol (ICN Biomedicals, St. Laurent, Quebec, Canada) and 1 mCi/ml of D-[1-¹⁴C]Gal (56 mCi/mmol; Amersham) to label gangliosides. After 24 hr, cells are washed with medium three times to remove unincorporated sugars, then cultured for an additional 24-48 hr in fresh medium, before harvesting by centrifugation at 400 g. Radioactivity in the tumor cell supernatant and cells is quantitated by liquid scintillation counting. The supernatant is clarified by centrifugation at 15,000 g for 10 mm, then concentrated 10-fold using a Speedvac concentrator, before being analyzed by gel filtration chromatography.

Gel Filtration Chromatography of ¹⁴C-Labeled Tumor Cell Supernatant on Sepharose 2B-300

Concentrated ¹⁴C-labeled tumor cell supernatant is chromatographed on a Sepharose 2B-300 column (5 ml bed volume; Sigma Chemical Go, St Louis, Mo.), equilibrated with Tris-buffered saline (TBS; 50 mM Tris-HCl in 0.15 M NaCl, pH 7.4). The column is eluted at a flow rate of 0.2 ml/min at 22° C., and 200 ml fractions are collected and counted for ¹⁴C. Dipalmitoylphosphatidylcholine liposomes and sodium azide are used as standards to calibrate the void and included volume of the column, respectively.

Gel Filtration FPLC of'P-Labeled Tumor Cell Supernatant on Superose

FPLC is carried out on a Superose 6 column (1×30 cm; Pharmacia, Dorval, Quebec, Canada) linked to a Gilson HPLC system and a Gilson iliB ultraviolet flow detector. The column is calibrated with a series of standard proteins of known molecular mass, ranging from b-galactosidase (465 kDa) to b-lactoglobulin (36.8 kDa) (Pharmacia, High Molecular Weight Gel Filtration Calibration kit). The void volume and included volume are determined using Blue Dextran (2000 kDa) and sodium azide, respectively. ³H-Labeled bovine brain gangliosides and [¹⁴C]Gal dissolved in NSLP or TBS are also used as standards. Concentrated YAC-1 supernatant is eluted through the column at 22° C. with TBS at a flow rate of 0.5 ml/min. Fractions (0.5 ml) are collected and counted for ¹⁴C.

### Example 43

Assessment of SAg and VT Binding to Glycosphingolipids by TLC Overlay

Glycolipids (dissolved in chloroform/methanol (2:1 v/v), are applied to a TLC plate and separated in choroform/methanol/water (65:25:4, v/v). Toxin binding is determined using known methods. Briefly, after separation of the glycolipids, the plate is air dried, incubated overnight at 37° C. in a solution of 1% (m/v) gelatin in 50 mM Tris/HCL, 150 mM NaCl, pH 7.4 (buffer A). The plate is washed in buffer A and incubated successively with VT 1 (0.07 mg/ml in buffer A) followed by monoclonal antibody PHI (1.5 mg/ml in buffer A), and finally with goat antimouse IgG horseradish peroxidase conjugate (diluted 1:2000 in buffer A). Toxin binding is visualized using 4-chloro-1-naphthol. An equivalent plate is run and treated with 3% (m/v) orcinol spray in 3 M H2SO4 to visualize carbohydrate and ensure equal concentrations.

Alternate Microtitre Plate Binding Assay

Quantification of toxin binding to various glycoconjugates is performed using published methods. A methanolic solution [100 pl containing glycolipid (300 nmol). phosphatidylcholine (0.5 mg) and cholesterol (0.25 mg)] is added to microplate wells and the methanol is allowed to evaporate overnight at room temperature. The wells are blocked with 2% (m/v) BSA in buffer A (200 ml/well) for 2 h at room temperature and subsequently washed once with buffer A containing 0.1% BSA (BSA/buffer A). 100 ml aliquots of dilutions of [¹²⁵I]-VT-1 in BSA/buffer A are added to the wells and incubated for 2 h at room temperature. The wells are washed five times with BSA/buffer A. excised and the radioactivity is measured in a g counter. Scatchard analysis was performed using the LIGAND program.

### Example 44

Methods of Induction and Assessment of Apoptosis & Inhibition of Protein Synthesis

Tumor cells (5×10⁵ cells/ml) arecultivated at 37° C. in 96-well round-bottomed microtiter plates (Becton Dickinson) in 200 ml leucine-depleted RPMI (Eurobio, France) containing 1 mCi of [³H] leucine. with or without 10 ng/ml VT. After 18 hrs. cells are harvested on class fiber filters, and radioactivity incorporated in proteins measured in a scintillation counter.

Ultrastructural Analysis of VT-Treated Astrocvtoma Cells

Cells are cultivated on a transferable 9 mm cylcopore membrane (0.45 mm pore size. Falcon) to form a confluent monolayer and are incubated at 37° C. with VTI (10 ng/ml). Cells are fixed at room temperature by addition of 1.6% glutaraldehyde to the wells and then incubated in 0.066 M Sorensen buffer (pH 7.4) containing 1.5% glutaraldehyde for 1h at 4° C. After 2 h of washing with 0.1 M phosphate buffer, cells are post-fixed in 2% osmium tetroxide in the same buffer. After dehydration in graded ethanols and propylene oxide, Epon embedding, thin sectioning and uranyl-lead counterstaining on grids are performed. Thin sections are examined in a Philips EM 400 electron microscope and ultrastructural features of apoptosis are analyzed

Flow Cytometry

Apoptosis of astrocytoma cells, incubated with 10 ng/ml of VT1 for 24-36 hrs in the presence of 10% bovine fetal serum is analyzed on an Epics Profile Analyzer (Coulter Electronics. Pathology. University of Toronto) according to known procedures. After treatment, cells are trypsinized and the 200×g centrifuged cell pellet is suspended in 1 ml of hypotonic fluorochrome solution of 50 mg/ml propidium iodide (Sigma) and stained for 30 min at 4° C. To remove RNA prior to staining. cells are treated with 100 ml of 200 mg/ml solution of DNase-free RNase A at 37° C. for 30 min. Cell cycle distribution is determined using manual gating. Flow cytrometric quantitation of apoptotic cells within the propidium iodide-stained population is performed as described. Debris and dead cells are excluded on the basis of their forward and side light-scattering properties. Astrocytoma cells grown simultaneously in the absence of VT1 serve as controls.

DNA Fragmentation Assays Cells:

Tumor cells are incubated in RPMI 1640 medium alone or in the presence of intact VT or VT-B. After 18-h culture, cells are counted and viability assessed by trypan blue exclusion. Cells are then centrifuged and washed twice with saline buffer. The pellets are lysed by incubation for 1 h at 50° C. in 10 mM EDTA, 200 mM NaCl, 0.1 mg/ml proteinase K, 0.5% (w/v) SDS, and 50 Mm Tris-HCL, pH 8. The DNA is extracted with phenol, chloroform:isoamylalcohol (24:1), and then ethanol precipitated. Unfragmented DNA is discarded, and 0.1 volume of 3 M sodium acetate, pH 7.2, is added to the supenatant which is left at −80° C. overnight. The precipitate containing fragmented DNA is centrifuged (1300 g, 30 mm) and dried under vacuum. DNA derived from 5×10⁶ cells is then resuspended in 20 ml RNAse buffer containing 0.5 mg/ml DNAse-free RNAse (Sigma), 15 mM NaCl, and 10 mM Tris-HCL, pH 7.5. and incubated at 50° C. for 1 h; Electrophoresis is carried out at 70V in 2% agarose gel containing 0.1 mg/ml ethidium bromide in a buffer containing 2 mM EDTA, 80 mM Tris-phosphate. pH 8. After electrophoresis. gels are examined under UV. Phage DNA from bacteriophage l and f digested by HindIII and HaeIII, respectively, provide molecular weight standards.

Nuclear Staining with Propidium Iodide

SF-539 cells grown on the cover slips overnight are incubated at 37° C. with VT-12B subunit (50 mg/ml) for 1.5 hrs or 10 hrs and fixed (with 1% parafornaldehyde for 3 minutes). permeabilized with 0.1% Triton X in 100 mM PBS for5 min, and stained with 5 mg/ml propidium iodide (Sigma). After extensive wash with 50 mM PBS, the fixed cells are mounted with DABCO (1,4-diazabicyclo-octane (Sigma), and nuclear staining is observed under incident UV illumination.

Proliferation Assay

Approximately 1-5×10⁴ cells are added to 24-well plates and incubated in a-MEM in 5% CO2 at 37° C. After 24 hr, the growth medium is replaced with medium containing various concentrations of the holotoxin VT1 (0.0.1.5.50, 100 ng/ml). The treated astrocytoma cell lines and endothelial cells are trypsinized and counted at intervals throughout the growth curve. Cell viability is assessed by trypan blue dye exclusion. Cell counts are plotted against time for the various concentrations of VT 1 and B subunit. For each time point analyzed, the wells are set-up in triplicate. For selected cell lines, the B subunit of VT1, VT2, and VT2c is added alone to the astrocytoma cells at same concentrations listed above. A single dose of VT1. VT2. and VT2c is added to confluent astrocytoma cells in microplate wells. Cell survival at 72 hr is monitored by staining with 0.1% crystal violet, and measuring the optical density at 590 nm using a Dynatek microtiter plate reader.

### Example 45

Multidrug Resistant Cells: Culture and Preparation

MCF-7-wt and MCF-7-AdR (adriamycin-resistant) cells are obtained from Drs. K. H.

Cowan and M. B. Goldsmith, National Cancer Institute. Cells are maintained in RPMI 1640 medium containing 10% FBS (v/v), 50 units/ml penicillin, 50 mg/ml streptomycin, and 584 mg/liter L-glutamine. KB-3-1 human oral epidermoid carcinoma cells (parent, * drug-sensitive) and KB-V-1 cells (highly MDR) and subclones are obtained from the National Cancer Institute). Cells are grown in high glucose (4.5 g/liter) Dulbecco's modified Eagle's medium containing 10% FBS and other components described above. The KB-V-1 cell line is maintained with vinblastine (1.0 mg/mi) in the medium. NIH:OVCAR-3 cells (human ovarian adenocarcinoma, drug-resistant) are obtained from the American Type Culture Collection and grown in RPMI 1640 medium containing insulin (10 mg/ml), 10% PBS, and other components listed above. All cells are cultured in a humidified, 6.5% CO2 atmosphere, tissue culture incubator. Cells are subcultured once a week using 0.05% trypsin and 0.53 mM EDTA solution.

Lipid Mass Analysis

Cell lipids are analyzed by TLC separation and charring of the chromatogram. Briefly, total cellular lipids are extracted and equal aliquots (by weight) from each sample are spotted on TLC plates. Plates are developed in the desired solvent system (see below), air-dried for 1 h, and sprayed using a 35% solution of sulfuric acid in water (v/v). The lipids are charred by heating in an oven at 180° C. for 30 mm, and resulting black bands are visualized.

Cell Radiolabeling and Analysis of Sphingolipids

MCF-7 cells grown in medium containing 10% FBS, are switched to serum-free medium containing 0.1% fatty acid-free BSA. Cell lipids are radiolabeled by incubating cells with [³H]serine (2.0 mCi/ml), [³H]palmitic acid (1.0 mCi/ml, or [³H]galactose 1.0 mCi/ml) for the indicated times. In some instances, cells are radiolabeled in medium containing 5% PBS. Cells are then rinsed twice with PBS, and 2 ml of ice-cold methanol containing 2% acetic acid is added. The cells are scraped free, transferred to glass test tubes (13×100 mm), and lipids are extracted by the addition of chloroform (2 ml) followed by water (2 ml). The resulting organic lower phase is evaporated under a stream of nitrogen. Lipids are resuspended in 100 ml of chloroform'methanol (1:1, v/v) and aliquots are applied to TLC plates. When using [³H]galactose, radiolabeled cells are washed twice with PBS, transferred to glass tubes with methanol (2 ml, and glucosylceramides and gangliosides (2.5 mg of each) are added to aid recovery. Lipids are extracted by the addition of water (2 ml; and 2 ml of chloroform (three times consecutively). The pooled organic lower phase is treated as above. Lipid analysis is carried out by various TLC separations using solvent system I, chloroform/methanol/ammonium hydroxide (65:25:5, v/v); solvent system II, chloroform/methanol/ammonium hydroxide (40:10:1, v/v), solvent system III, chloroform/methanol/water (60:40:8, v/v), or solvent system IV, chloroform/methanol/acetic acid/water (50:30:7:4, v/v). For determination of ceramides. an aliquot of the chloroform-soluble lipids is base-hydrolyzed in 0.1 N KOH in methanol for 1 h at 37° C.; lipids are re-extracted and separated using solvent system V hexane/diethyl ether/formic acid (60:40:1, v/v). Galactosyl- and glucosyl-ceramides are separated using solvent system VI, chloroform/methanol/water (60:25:4, v/v). This separation is performed on TLC plates that are pre-run in 2.5% borax in methanol/water (1:1) and heated at 110° C. prior to use.

Radiochromatograms are sprayed with EN³HANCE and exposed for 3-7 days for autoradiography. TLC areas, aligned with hands on the autoradiographs or with iodine-stained commercial lipid standards are scraped from the plate. Water (0.5 ml) is added to the plate scrapings, followed by 4.5 ml of EcoLume counting fluid, and the samples are quantitated by liquid scintillation spectrometry.

Purification of Glyosylceramides

The compounds, extracted with total lipids from MCF-7-AdrR cells, are resolved from other lipids on preparative TLC using silica gel H plates developed in solvent system II. The appropriate region of the TLC plate is then scraped into test tubes, and lipids are extracted with chloroform/methanol/acetic acid/water (50:25:1:2, v/v). The samples are centrifuged, and the solvent transferred to new glass tubes and evaporated to dryness under nitrogen.

Fast-Atom Bombardment/Mass Spectrometry of TLC-isolated Lipid-

FAB/MS spectra are acquired using a VG 70 SEQ tandem hybrid instrument of EBqQ geometry (VG analytical, Altrincham, UK.). The instrument is equipped with a standard unheated VG FAB ion source and a standard saddle-field gun (Ion Tech Ltd., Middlesex, UK) that produces a beam of xenon atoms at 8 kV and 1 mA. The mass spectrometer is adjusted to a resolving power of 1000, and spectra are obtained at 8 kV using a scan speed of 10 s/decade. 2-Hydroxyethyl disulfide is used as matrix in the positive FAB/MS, and triethanolamine is used as a matrix in the negative FAB/MS. Negative FAB and positive FAB give different values for the same compounds, due to charge (proton content) differences.

### Example 46

Incubation of Tumor Cells with Hydroxy Fatty Acids for Selective Synthesis of Galactosphingolipids and Lipid Analysis

Tumor cells on filters are incubated for 1 hr at 37° C. in the presence of labeled and unlabelled [³H]Cer(C6[D-20H]). After the incubation, lipids are extracted from the cells and the combined incubation media and analyzed Lipids are extracted from cells and media by a two-phase extraction. The upper phase contains 20 mM acetic acid and (for radiolabeled lipids) 120 mM KCl. After a chloroform wash. which is added to the lower phase, lipids remaining in the upper phase GalCer are collected on SepPak C18 cartridges (Waters, Milford, Mass.) from which lipids are eluted with chloroform/methanol/water 1:22:0.1) and methanol. The organic (lower) phase is dried under N2, and the lipids are applied to TLC plates that were dipped in 2.5% boric acid in methanol, dried, and activated by heating at 110° C. for 30 mm. They are developed in two dimensions:

I. chloroform/methanol/25%NH4OH/water (65:35:4:4. v/v); and

II. chloroforrm/acetone/methanol, acetic acid/water (50:20:10:10:5. v/v).

Fluorescent spots are detected under UV. scraped from the TLC plates and the fluorescent lipid analogs are extracted from the silica in 2 ml chloroform/methanol/20 mM acetic acid (1:2:2:1 v/v) for 30 mm. After pelleting the silica for 10 min at 1,500 rpm fluorescence in the supernatants is quantified in a fluorimeter (Kontron. Zorich, Switzerland). Radiolabeled spots are detected by fluorography after dipping the TLC plates in 0.4% PPO in 2-methylnaphthalene with 10% xylene. Preflashed film (Kodak X-Omat S) is exposed to the TLC plates for 3 d at −80° C. The radioactive spots are scraped from the plates, and the radioactivity is quantified by liquid scintillation counting in 0.3 ml Solulyte (J. T. Baker ChemicaL Deventer, The Netherlands) and 3 ml of Ultinsa Gold (Packard Instruments. Downers Grove. Ill.).

### Example 47

Conjugation of Proteins to Lipoproteins

The preferred method for coupling superantigens to lipoproteins is to use 10 mM solution of sodium periodate for oxidation of the carbohydrate in the lipoprotein. This will also cleave c-c bonds in the sugars with adjacent hydroxyls and oxidize them to reactive aldehydes. Superantigens form Schiff base linkages with the aldehyde modified sugar groups under alkaline conditions. the aldehyde modified sugar is then coupled to the amine containing superantigen peptide or polypeptide. The oxidation is followed by reductive amination using sodium cyanoborohydride to reduce the labile Schiff base between the aldehyde on the carbohydrate and the amine on the superantigen to form stable secondary amine covalent linkages.

An alternative procedure is to periodate oxidize the lipoprotein as above to create reactive aldehyde groups. Heterobifunctional cross-linking agent such as 4-(4-N-Maleimidophenyl)butryric acid hydrazide (MPBH) 4-(4-N-Maleimidophenyl)buryric acid hydrazide (MPBH), and 4-(N-Maleimidomethyl)cyclohexane-1-carboxyl-hydrazide (M2C2H) which contain a carbonyl-reactive hydrazide group on one end and a sulfhydryl-reactive maleimide on the other are preferred. The hydrazide reacts specifically with aldehyde functional groups to create a hydrazone linkage a type of Schiff base. To stabilize the bond between the hydrazide and aldehyde, the hydrazone is reacted with sodium cyanoborohydride to reduce the double bond and form a secure covalent linkage. The cross-bridge between the two functional ends provides a long, 17.9-A spacer. These agents couple to periodate-oxidized aldehydes on the lipoportein carbohydrate via the hydrazine and to sulfhydryl groups on the superantigen via sulfhydryl reactive maleimide group. Superantigens without reactive sulfhydryl groups are first thiolated with SATA or Trout's reagent before addition to the reactive maleide. A sulfhydryl-containing protein or molecule is is bound via the maleimide end of MPBH and the derivative purified by gel filtration to remove excess reactants, and then mixed with a lipoprotein (that had been previously oxidized to provide aldehyde residues) to effect the final conjugation. The opposite approach e.g., modification of the glycoprotein first, purification, and subsequent mixing with a sulfhydryl-containing molecule is also acceptable. With this second option, however, the purification step should be done quickly to prevent extensive hydrolysis of the maleimide group. (See Hermanson GT Bioconjugate Techniques Academic Press, San Diego Calif., 1996)

Protocol for Periodate Oxidation

1. Periodate-oxidize a liposome suspension containing glycolipid components according to Section 2. Adjust the concentration of total lipid to about 5 mg/ml.

2. Dissolve the protein to be coupled in 20 mM sodium borate, 0.15 M NaCl, pH 8.4,at a concentration of at least 10 mg/ml.

3. Add 0.5 ml of protein solution to each milliliter of lipoprotein suspension with stirring.

4. Incubate for 2 h at room temperature to form Schiff base interactions between the aldehydes on the lipoprotein and the amines on the protein molecules.

5. In a fume hood, dissolve 125 mg of sodium cyanoborohydride in 1 ml water (makes a 2 M solution). This solution may be allowed to sit for 30 mm to eliminate most of the hydrogen-bubble evolution that could affect the lipoprotein suspension.

6. Add 10 ul of the cyanoborohydride solution to each milliliter of the lipoprotein reaction.

7. React overnight at 4° C.

8. Remove unconjugated protein and excess cyanoborohydride by gel filtration using a column of Sephadex G-50 or G-75.

### Example 48

Isolation of Lipoproteins

Human LDL is isolated by sequential ultracentrifugation (d 1.019-1.063 g/ml) from freshly drawn, citrated normolipidemic human plasma to which EDTA 0.1 mmol/liter is added. Freshly obtained plasma is subjected to differential ultracentrifugation to isolate the desired lipoprotein fractions. Typically, the following density fractions were isolated: 1) d<1.02. to remove VLDL and IDL; 2) d=1.02-1.05, to obtain LDL; 3) d=1.05-1.08, to obtain Lp(a); and 4) d=1.08-1.21, to obtain Lp(a) and HDL. The Lp(a)-containing density fractions were subjected to gel filtration chromatography on a Bio-Gel A-15 m column (2.5×90 cm). This column was eluted with 1.0 M NaCl, 10 mM Tris, 10 mM NaN3, I mM EDTA, pH 7.4, and was continuously monitored at 280 nm. The LDL- and HDL-containing density fractions are also subjected to gel filtration chromatography to remove any contaminating species and for uniformity of sample preparation. They are further dialyzed against 0.01 M sodium phosphate pH 7.4, containing 0.15 M sodium chloride and 0.01% EDTA, sterilized on 0.2-um Millipore membrane, and stored at 4° C. under nitrogen (up to 3 weeks).

Lipoprotein (a) (Lp(a))

Lp(a) is prepared from fresh human plasma by flotation centrifugation followed by affinity chromatography on lysine-Sepharose and CsCI density gradient centrifugation as described. Lipoprotein preparations are dialyzed against 0.15 M sodium chloride containing 0.01% EDTA at 0.01% sodium azide, filter sterilized (0.45 pm) and stored at 4° C. in vials filled to allow no air space. No contamination of the preparations by plasminogen is detected by either Coomassie Blue staining of sodium dodecyl sulfate (SDS) gels or by treatment with streptokinase and measuring plasmin activity with a chromogenic substrate. S2251. The sensitivities of these assays excluded plasminogen contamination of>1% and>0.4% respectively. Lp (a)-free LDL, HDL and acetylated LDL are prepared as previously described. The LDL contained no apoA-1 and the HDL contained no detectable apoB-100. The apoprotein composition is verified by SDS polyacrylamide gel elecrophoresis.

Lysine-Sepharose Chromatography

Lipoprotein (a) has an affinity for lysine-Sepharose by virtue of lysine binding kringle 4 domain(s) located on apo(a). The most important domain appears to be kringle 4₃₇, which has the greatest homology to kringle 4 of plasminogen, although there may be other kringles with lesser affinity for lysine which also contribute to the interaction of Lp(a) with lysine-Sepharose. Plasminogen and Lp(a) have similar affinities for lysineSepharose; however, Lp(a) species with different apo(a) isoforms may have affinities that are significantly greater or weaker than that of plasminogen. The buffer of choice in the isolation of plasminogen from plasma by lysine-Sepharose affinity chromatography has been 0.1 M phosphate buffer, pH 7.4. When the same buffer system is used in the chromatography ofLp(a), not all the lipoprotein is found to bind to the lysine-Sepharose i.e., approximately 80% of Lp(a) contained in the plasma had the capacity to interact with lysine-Sepharose. The percentage of Lp(a) binding to lysine-Sepharose is increased by lowering the ionic strength of the buffer medium. Lipoprotein (a) species with large apo(a) isoforms tend to self-associate in the cold therefore it is best to perform the chromatographic isolation at room temperature.

Preparation of Lysine-Sepharose 4B

Packed Sepharose 4B (250 ml) is washed with 8 liters of water on a coarse sintered glass funnel and activated with 25 g CNBr dissolved in 50 ml acetonitrile. The reaction is carried out in a well-ventilated hood, on ice, and the pH is maintained with 6 N NaOH at pH 11. After approximately 15 to 30 mm, the activated Sepharose 4B is washed with 8 liters of 0.1 M NaHCO₃ pH 8.1. The agarose is then packed by filtration, diluted with 250 ml of 0.1 M NaHCO₃ pH 8.1, containing 50 g lysine, and stirred gently overnight at 4°. The freshly conjugated lysine-Sepharose is then washed with 6 to 10 liters of 1 mM HCl followed by 8 liters of 0.1 M NaHCO3, pH 8.1, and an aliquot is saved for determination of the concentration of immobilized lysine residues using the method of Wilkie and Landry. The concentration of coupled lysine varies from 15 to 25 umol per milliliter packed gel

Chromatography

Bio-Rad (Richmond, Calif.) Econo-Pac columns (I×12 cm) are packed with 5 ml lysine-Sepharose which is preequilibrated with column buffer (e.g., 0.1 M phosphate, 0.01% NaN₂, pH 7.4). A porous polymer filter is placed on top of the lysine-Sepharose gel bed to prevent the column from running dry. Plasma samples smaller than 3 ml are applied to the column and allowed to run through by gravity at room temperature. Larger volumes (up to 50 ml) should be applied with a pump or by gravity, but at flow rates that should not exceed 20 ml/cm²/hr. The samples are washed into the column with four 0.5-ml aliquots of column buffer to be followed with four 0.5 ml aliquots, before Lp(a) is eluted withe0.2<EACA in 10 mM phosphate, pH 7.4. One milliliter aliquots are applied at a time, and 1 ml fractions are collected in separate tubes. Liprotein(a)and plasminogen-containing fractions (tubes 4 through 10) are located by their absorbance at 280 nm. The volume of applied plasma depends on the Lp(a) content and on the sensitivity of the absorbance monitor that is part of the density gradient fractionating system.

Density Gradient Centrifugation of Lp(a)

Place 5 ml of 20% (w/w) NaBr into a SW-40 ultracentrifuge tube (ultraclear). Carefully layer the eluate from the lysine-Sepharose column (up to 8 ml) on top of the NaBr solution and, if necessary, top off the tube with 0.2 M EACA, 10 mM phosphate, pH 7.4. Place the tubes in the bucket of the swinging-bucket rotor and centrifuge 64 hr at 39,000 rpm and 20°. After centrifugation is completed, the tubes are carefully removed from the buckets and placed in the density gradient fractionating system. The tubes are pierced at the bottom, and the gradient is pushed out the top at a flow rate of 1 ml/min with a dense fluorocarbon oil, Fluorinert FC-40 (ISCO), that has a density of 1.85 g/ml. The chart speed is 1 cm/min, and the fraction collector is set to 0.5 ml/tube. The gradient is monitored at 280 nm, and the sensitivity of the chart recorder is adjusted according to the Lp(a) content of the eluate. Densities of the various fractions are measured with a density meter by established techniques.

Isolation of Apolipoproteins B-48 and B-100

The following density gradient ultracentrifugation procedure for isolating subfractions of triglyceride-rich lipoproteins is suitable for SDS-PAGE on both slab and rod gels. Plasma is recovered by low speed centrifugation (1750 g, 20 min, 10). To minimize proteolytic degradation of apo B, 1.0 ul/ml plasma phenylnethylsulfonyl fluoride (PMSF, Sigma, St. Louis, Mo.), 10 mM dissolved in 2-propanol, and 5 ul/ml plasma aprotinin (Trasylol, Bayer, Leverkusen, (Germany), 1400 ug/liter, are added. Subsequently 140.4 mg solid NaCl is added per 1.0 ml plasma to increase the density to 1.10 kg/liter. Normally, a total volume of 4.0 ml of the d 1.10 kg/liter plasma is put in the bottom of a 13.4-ml polyallomer ultracentrifuge tube (Ultra-Clear, Beckman Instruments, Palo Alto, Calif.). Alternatively, 3.0 ml plasma can be mixed with 1.5 mIt) 1.42 kg/liter NaUr, from which 4.0 ml is transferred to the ubracentrifuge tube. For the rod gel method, two such tubes are required to obtain enough material from each sample. For the slab gel method, 1.0 ml plasma is sufficient. In the latter case, a 1.0 ml portion of 1.10 kg/liter plasma can be mixed with 3.0 ml of 1.10 kg/liter NaCl in the tube. A density gradient consisting of 3.0 ml each of 1.065, 1.020, and 1.006 kg/liter NaCl solutions is then sequentially layered on top of the plasma.

Ultracentrifugation is performed in a SW4O Ti swinging bucket rotor (Beckman) at 40,000 rpm and 15° (Beckman L8-55 ultracentrifuge). Consecutive runs calculated to float Svedberg flotation rate (Sf)>400 (32 min), SI 60-400 (3 hr 28 mm), and Sf 20-60 (14-16 hr) particles are made. After each centrifugation, the top 0.5 ml of the gradient containing the respective lipoprotein subclasses is aspirated, and 0.5 ml of density 1.006 kg/liter salt solution is used to refill the tube before the next run. The Sf1 12-20 fraction is recovered after the last ultracentrifugal run by slicing the tube 29 mm from the top after the Sf 20-60 lipoproteins have been aspirated. All salt solutions should be adjusted to pH 7.4 and contain 0.02 % (w/v) NaN3 and 0.01% Na2EDTA. This method yields lipoprotein preparations almost completely devoid of plasma albumin.

### Example 49

Preparation & Isolation of Oxidized LDL (OxyLDL Oxidized LDL (OxyLDL)

Native LDL (200 ug protein/ml) is oxidized by exposure to 5 uM CuSO4 for 24 h at 25° C. and the degree of oxidation is assessed by the increase of mobility on 1% agarose gel (1.3-1.5 versus native LDL) and the formation of thiobarbituric acid-reactive substances (3.41+/−0.8 mmol/L). Oxidation is terminated by refrigeration. Different preparations of oxyLDL display similar electrophoretic mobilities. For comparison, commercially available preparations of native and copper-oxidized LDLs (Sigma Chemical Co., St. Louis, Mo. and Biomedical Technologies, Inc.. Stoughton, Mass., respectively) are used. The level of LDL oxidation is evaluated by monitoring the formation of lipid hydroperoxides, using the FOX-2 procedure and thiobarbituric acid-reactive substances (TBARS)). The relative electrophoretic mobility is evaluated on Hydragel (Sebia, Paris, France) and the level of trinitrobenzenesulfonic acid-reactive amino groups was determined

The formation of thiobarbituric acid-reactive substances is 17.8 nanomoles of malondialdehyde/mg protein using an oxyLDL preparation with relative electrophoretic mobility of 1.4.

Methods for Measurement of Low-Density Lipoprotein Oxidation

Oxidation of LDL in vitro is accompanied by characteristic changes of chemical, physicochemical, and biological properties, and a variety of methods may therefore be used for determining the extent and/or rate of oxidation of LDL. They include measurement of the increase of thiobarbituric acid-reactive substances (TBARS), total lipid hydroperoxides defined lipid hydroperoxides, hydroxy and hydroperoxy fatty acids, conjugated dienes, oxysterols, lysophosphatides, aldehydes and fluorescent chromophores as well as measurements of the disappearance of endogenous antioxidants and polyunsaturated fatty acids, and oxygen uptake. The apolipoprotein B (apoB) becomes progressively altered during oxidation; its loss of reactive amino groups and fragmentation to smaller peptides is determined and used as an index of oxidative modification. The net increase of the negative surface charge of the whole LDL particle is analyzed as relative electrophoretic mobility (REM) by agarose gel electrophoresis. The biological assays used most frequently for assessment of the extent of oxidative modification are the rate of uptake of LDL by cultured macrophages and its cytotoxicity toward cultured cells. Immunological assays such as enzyme-linked immunosorbent assay (ELISA) and radioimmnunoassay (RIA) employing polyclonal or monoclonal antibodies recognizing certain modifications in apoB characteristic for oxidative modification are employed. The epitopes produced by covalent binding of malonaldehyde or 4-hydroxynonenal are of particular interest. Nuclear magnetic resonance (NMR), electron spin resonance (ESR), circular dichrorism (CD), and fluorescence polarization have also been applied to study certain aspects of LDL oxidation. Simple methods, such as the measure of TBARS, conjugated dienes, or fluorescence are preferred. Most characterize oxidized LDL by at least two independent measurements, for example, TBARS or REM and macrophage uptake, antioxidants and conjugated dienes.

From kinetic experiments one can conclude that both cell-mediated oxidation of LDL and oxidation in the absence of cells catalyzed by Cu²⁺ ions proceed in three consecutive time phases: lag phase, propagation phase, and decomposition phase. 1)during the lag phase the LDL becomes depleted of antioxidants, and during this period only minimal lipid peroxidation occurs in LDL, as shown by measuring polyunsaturated fatty acids (PUFAs), TBARS, lipid hydroperoxides, fluorescence, and conjugated dienes. When LDL is depleted of its antioxidants, the rate of lipid peroxidation rapidly accelerates and a lipid peroxide maximum is reached after about 70-80% of the LDL, PUFAs are oxidized. Thereafter, the peroxide content of LDL, starts to decrease again because of decomposition reactions. During the lag and propagation phases the time profile for TBARS, fluorescence at 430 nm, lipid peroxides, dienes, and REM are very similar and only after the peroxide maximum do the different indices separate and follow different kinetics. This also indicates that all the methods will give equivalent results for the susceptibility of LDL to oxidation as measured by the duration of the lag time.

Preparation of Low-Density Lipoproteins for Oxidation Isolation of Low Density Lipoproteins

After overnight fasting blood samples are withdrawn by venipuncture and collected by free flow of blood into plastic tubes containing the appropriate volume of an aqueous solution of 10% EDTA (w/v) (disodium salt, pH 7.4) to obtain a final blood concentration of 0.1% EDTA (wlv). EDTA serves as anticoagulant and antioxidant. Blood is centrifuged at 1000 g for 10 mm; the supernatant is then centrifuged at 10° C. and 1000 g for 5 min, followed by centrifugation at 15,000 g for 10 min. This procedure removes all cellular debris, and a completely clear plasma is obtained. Generally plasma is not stored but is used the same day for LDL. isolation. The most common method for isolation of LDL is a two-step sequential ultracentrifugation with a total run duration of about 48 hr. LDL is prepared for oxidation experiments by a single 20-hr run with a discontinuous density gradient. Plasma (up to 4 ml) adjusted with solid KBr to a density of 1.22 g/liter is layered on the bottom of a centrifuge tube (Beckman polyallomer tubes, total volume 13.2 ml) and then overlaid by KBr density solutions of 1.08 (3 ml), 1.05 (3 ml), and 1.00 g/liter (to fill the tube) containing 1 g/liter EDTA (pH 7.4). All density solutions are purged with nitrogen before use. The tubes are centrifuged in a Beckman SW 41 Ti rotor at 40,000 rpm at 10° for 20 hr. After centrifugation the main lipoproteins very low-density lipoproteins (VLDL), LDL, and high-density lipoproteins (HDL) are well separated from each other, and the LDL band characterized by the yellow color due to the endogenous b-carotene, is collected by aspiration with a syringe and transferred into a polycarbonate tube.

Next, the cholesterol content of the isolated LDL sample is determined with the CHOD-PAP enzymatic test kit (Boehringer, Mannheim, Germany). When 4 ml normolipidemic plasma is centrifuged, the final LDL stock solution harvested from the ultracentrifugation has a concentration of total cholesterol of about 1.6 to 2.2 mg/ml. Based on the known composition of LDL the total cholesterol values can be converted to LDL mass per milliliter (multiply cholesterol by the factor 3.16) or LDL protein per milliliter (multiply total cholesterol by the factor 0.63). It is also possible to determine the LDL concentration by protein measurement. Next EDTA is from the LDL stock solution and the oxidation is conducted immediately after isolation of LDL. For storage the LDL stock solution is sterile filtered through a 0.3 um filter adapted to a syringe into a sterile, evacuated glass vial and subsequently purged with nitrogen (Techne Vial, Mallinckrodt-Diagnostica, Holland, or Behring, Marburg, Germany).

Removal of EDTA

Removal of EDTA and salt from the density gradient from the LDL stock solution is conducted with prepacked columns (Econo-Pac 10DG, Bio-Rad, Richmond, Calif.) filled with Bio-Gel P6 as desalting gel. The bed volume is 10 ml with a void volume of 3.3 ml, and the total column volume is 30 ml. The gel is preconditioned by passing 20 ml phosphate-buffered saline (PBS, 10 ml sodium phosphate buffer, pH 7.4, containing 0.15 M sodium chloride) through the column.

A volume of 0.5 ml of the LDL stock solution is then applied to the column. After the LDL solution has run into the gel, 2.5 ml PBS is applied. The first 3 ml of eluate are discharged. The column is then eluted with 1 ml PBS, and 1 ml EDTA-free LDL solution is collected in a 1.5-ml Eppendorf vial. The vial is immediately made oxygen-free by nitrogen gassing and transferred to a refrigerator. An aliquot is removed to determine again the concentration by the CHOD-PAP method. The LDL solution can be rather unstable at this stage, depending on the donor, and therefore the time elapsed between desalting and the final oxidation experiment should not exceed 60 mm.

Thiobarbituric Acid-Reactive Substances as Index of Low-Density Lipoprotein Oxidation

The preferred assay in LDL oxidation studies, both in presence and absence of cells, is the determination of thiobarbituric acid (TBA)-reactive substances (TBARS) by one of the TBA assays developed for lipid peroxidation studies. The basal value of TBARS in freshly prepared LDL samples is usually low (0.5 to 3 nm/mg LDL protein) or undetectable. In LDL oxidized for about 24 hr with cells or CU²⁺ ions, the TBARS are in the range of 30 to 100 nmol/mg protein. In copper-stimulated oxidation, formation of TBARS shows a lag phase of about 40-150 min depending on the LDL, temperature, medium, and Cu2+ concentration; during this lag phase TBARS do not increase. Thereafter, TBARS rapidly increase for about 1-2 hr to a plateau value. On prolonged incubation TBARS remain more or less constant or increase slightly. The reported time course studies for TBARS in cell-mediated oxidation suggest that oxidation proceeds similarly to Cu²⁺ oxidation, with a lag phase followed by a rapid increase to a plateau level. In this context, it should be noted that most researchers only determine TBARS as an end point after about 24 hr incubation, when LDL has reached a final stage of oxidation.

Assays Used for Measurement of TBARS

Specifically, 100 ul of an LDL preparation (50 ug LDL cholesterin or 150 ug protein) is added to 1 ml of 20% trichloroacetic acid (TCA). Following precipitation, 1 ml of 1% thiobarbituric acid (TBA) is added, and the mixture is heated 45 min at 95°, cooled on ice, and centrifuged (20 min at 1000 g). TBARS are then determined by measuring the absorbance at 532 nm or the emission fluorescence at 553 nm (excitation 515 nm). Calibration is done with a malonaldehyde standard prepared from tetramethoxypropane. The second assay is typically as follows: LDL (25 ug protein) is mixed with 1.5 ml of 20% TCA and 1.5 ml of 0.67% TBA. After heating at 100° for 30 mm, TBARS are determined fluorimetrically at an emission wavelength of 553 nm with excitation at 515 nm. The sensitivity was reported to be 0.1 nmol TBARS/assay. This is equivalent to 4 nmol TBARS/mg protein. Haberland a al. determined the malonaldehyde-LDL adduct using a TBA assay. The malondialdehyde (MDA-treated LDL was precipitated with heparin-manganese, the supernatant was discharged after centrifugation, and the precipitate was washed with heparin-manganese prior to the TBA test.

Minimally modified LDL (MM-LDL) is prepared by dialyzing native LDL against 9 uM FeSO₄ in PBS for 72 h at 4° C. The electrophoretic mobility increased 1.1 to 1.2 versus native LDL. Mildly oxidized LDL was also obtained by (UV+copper/EDTA)-mediated oxidation under mild conditions: LDL solution (2 mg of apo/B/mi containing 2 umol/liter CuSO4) was irradiated for 2 h. as a thin film (5 mm) in an open beaker placed 10 cm under the UV-C source (HNS 30W OFR Osram UV-C tube, 1ₘₐₓ254 nm, 0.5 milliwatt/cm² determined using a Scientech thermopile Model 360001), under the standard conditions. At the end of the irradiation, aliquots were taken up for analyses and oxidized LDL (200 ug of apoB/ml under standard conditions or at the indicated concentration) were immediately incorporated in the culture medium.

Acetylation of LDL is performed with excess acetic anhydride. Endotoxin contamination in oxyLDL is measured with the coagulation Limulas arnebocyte lysate assay using a commercially available kit (E-TOXATE, Sigma Chemical Co.).

Induction of Apoptosis by oxyLDL

Incubation of HUVEC with oxLDL for 18 hours induced DNA fragmentation in a concentration-dependent manner with maximal effects at 10 ug/mL. In contrast, native LDL did not induce apoptosis in the concentration range tested. The induction of apoptosis by oxyLDL is confirmed by demonstrating DNA fragmentation through agarose gel electrophoresis. LDH release did not increase ³10 ug/mL oxLDL (105±11% compared with control cells) excluding the induction of necrosis

Detection of Fas and FasL Expression on Endothelial Cells

90% confluent HAECs and HUVECs were incubated with oxyLDL (150 ug protein/ml) or L-a-palmitoyl lysophosphatidyleholine (LPC, 45 uM, Sigma Chemical Co.) at 37° C., 5% CO2 for 13 h, and detached from the culture plate with 0.5% EDTA. To determine FasL expression, endothelial cells are incubated with an anti-FasL antibody (C-20, Santa Cruz Biotechnology, Santa Cruz, Calif.) or with rabbit IgG followed by a FITC-conjugated antibody against rabbit Ig (Biosource, Camarillo, Calif.). To determine Fas expression, endothelial cells were incubated with an FITC-conjugated anti-Fas monoclonal antibody (clone UBZ. Immunotech. Wemtbrook. Me.) or an FITC.conjugated mouse IgG. Immunofluorescence staining was analyzed by FACS (fluorescence-activated cell sorter) (Becton Dickinson. Mountain View, Calif.).

Detection of DNA Fragmentation by Agarose Gel Electrophoresis

HUVECs (10⁶) wcre incubated in the presence or absence of native LDL (300 ug protein/ml). oxyLDL (300 ug protein/ml). LPS (100 endotoxin U/ml), or a neutralizing anti-FasL antibody (i0 ug/ml, 4H9, MBL, Nagoya. Japan) for 36 h. Attached cells and floating cells were combined and lysed in 0.33 ml of lysis buffer (10 mM Tris-HCi. pH 8.0, 1 mM EDTA, 0.2% Triton X-100) followed by incubation with 0.1 mg/ml RNAase A for 1 h at 37° C. and 0.2 mg/mi proteinase K for 3 h at 50° C. Ethanol-precipitated DNA was resuspended in TE buffer, fractionated on 1.5% agarose gel in IX TBE buffer, and stained with ethidium bromide.

Detection of DNA Fragmentation by TdT-Mediated dUTP Nick-End Labeling (TUNEL).

70% confluent HUVECs are incubated in the presence or absence of OxLDL (300 ug protein/ml), a neutralizing anti-FasL antibody (10 ug/mi. 4H9). or an agonistic anti-Fas antibody (0.5 ug/ml CH11, MBL) for 16 h at 37° C., 5% CO₂. Attached cells harvested by trypsinization and floating cells are combined, fixed in 4% paraformaidehyde, permeabilized in 0.1% Triton X-100, 0.1% sodium citrate, and incubated with TUNEL solution (Boehringer Mannheim. Indianapolis. Ind.) in the absence or in the presence of terminal deoxynucleotidyl transferase. After washing in PBS, fluorescence intensity was analyzed by FACS.

Cell Viability Assay

HAECs or HUVECs are cultured in a 96-well plate at 80% confluency and incubated in the presence or absence of oxyLDL (300 ug protein/ml), LPC-C16:0 (55 uM). a neutralizing anti-FasL antibody (10 ug/ml. 4H9). or an agonistic anti-Fas antibody (0.5 ug/mi. CH11) for 18 h. Cell viability is measured by means of MTT (dimethyithiazol-diphenyltetrazolium bromide) assay and percentage of cell death was calculated as 100×(1−viability of treated endothelial cells/viability of untreated endothelial cells).

Cell Viability Assay and Reagents—Human umbilical vein endothelial cells (HUVECs) are isolated and cultured in endotheial growth medium (SCM; Clonetics, San Diego, Calif.). HUVECs cultured in a 96-well plate at 80% confluency are incubated with oyxLDL or LPC at indicated doses for 16 h. Cell viability is measured by means of MTT (3-(4,5-dimethyl thiazol-2-yl)-2,5-diphenyl tetrazolium bromide) assay

### Example 50

Preparation of Vesicles Expressing Recombinant Membrane Bound Superantigens Using the Yeast Sec6 Mutant

The superantigen cDNA, or oxyLDL receptor, apoprotein verotoxin or other polypeptide given herein corresponding to the cDNA for protein expression in yeast is used. The length of the 5′-untranslated region is minimized. Expression of a cDNA in the sec6-4 yeast mutant is best controlled and may be maximized with an inducible promoter. The GALl promoter is preferred. The pYES2 expression vector (InVitrogen, San Diego, Calif.) contains the GALI promoter followed by a multiple cloning site. Other commonly used inducible promoters include the metallothionein CUPI promoter, which is tightly controlled by copper; promoters activated in response to heat shock, which are of particular interest for expression in the temperature-sensitive sec6-4 mutant and the PH05 promoter, which is derepressed at low phosphate concentrations. Introduction of the plasmid into yeast cells is accomplished either by electroporation or LiCi-mediated transformation. Isolation of transformants requires selection yeast that are ura3 auxotrophs are able to grow on media lacking uracil when they contain the pYES2 expression vector that contains the wild-type URA3 gene. Other selectable markers include enzymes in the adenine, histidine, leucine, lysine, and tryptophan biosynthetic pathways. The superantigen cDNAs are cloned into the pYES2 expression vector and selected for transformants on plates with synthetic complete (SC) medium lacking uracil but containing 2% raffinose as the carbon source (SC-Ura raff medium). Single colonies are isolated and grown overnight to saturation in 2 ml of SC-Ura raff medium at 25° with constant shaking in 2% raffinose instead of glucose. In a subsequent step the yeast are switched to medium containing galactose as the carbon source as the GALl promoter initiates gene expression only when galactose is the predominant carbon source. The 2-ml starter culture in SC-Ura raff medium is added to a 1-liter culture of the same growth medium and incubated at 25° with constant shaking. When these cultures reach an 0D600 (optical density at a wavelength of 600 nm) of about 1.0 (usually about 12 hr), the cultures are centrifuged at 4000 g at 4° for 5 min, resuspended in 4 liters of SC-Ura gal induction medium (containing 2% galactose instead of 2% raffinose as the carbon source), and shifted to 37° for 2-3 hr to induce protein expression in the sec6 vesicles.

Following growth at 37°, the cells are collected by centrifugation at 4000 g at 40 for 5 mm and washed once in ice-cold water. Pellets are resuspended in an absolute minimum volume of water and quick frozen in liquid nitrogen. Cultures may then be stored indefinitely at −70°. Thawed cultures are resuspended to a final concentration of 50 OD618˜units/ml (e.g., a 1-liter culture at 0D600=1.0 is resuspended in 20 ml) in 10 mM dithiothreitol (DTF) and 100 mM Tris-CI, pH 9.4. The resuspended culture is shaken gently at room temperature for 10 min. This step increases the efficiency of spheroplast lysis at a later step by reducing disulfide bonds in the yeast cell wall. We then collect the cells by centrifugation at 4000 g at 4° for 5 mm and resuspend them in spheroplast buffer to a final concentration of 50 0D600 units/ml. Spheroplast buffer consists of 1.4 M sorbitol, 50 mM K2HPO4, pH 7.5, 10 mM NaN3, and 40 mM 2-mercaptoethanol. Spheroplasts are generated by digesting the cell wall with lyticase (or zymolyase) for 45 min at 37°, The amount of bacterially expressed, recombinant lyticase needed to form spheroplasts is determined empirically; after 45 min the OD₆₀₀ of a 10-ul aliquot of the yeast suspension diluted into 1 ml of 0.1% sodium dodecyl sulfate (SDS) should be ˜20% of the OD₆₀₀of the initial dilution measured at 0 min. The spheroplasts are then harvested at 3000 g for 5 min at 4, and the cells are resuspended gently with a pipette or Teflon rod in spheroplast buffer containing 10 mM MnCl2 to a final concentration of 50 OD₆₀₀ units/ml. Concanavalin A (Sigma, St. Louis, Mo.) is then added to a final concentration of 0.78 to 1.25 mg/ml and incubated with rotation or gentle shaking at 4° for 15-30 mm. A concanavalin A stock solution (25 mg/ml) is prepared in spheroplast buffer containing 1 mM MnCl2 and 1 mM CaCl₂ and is frozen in 1-mi aliquots. Lectin-coated spheroplasts are harvested at 3000 g for 5 mm at 4° and then resuspended in lysis buffer to a final concentration of 60-70 OD₆₀₀ units/mi. The suspension is homogenized using the loose pestle of a Dounce homogenizer and 30-40 strokes of the pestle at 40 (or on ice). Lysis bufferconsists of 0.8 M sorbitol, 10 mM triethanolamine (TEA), and 1 mM EDTA. The pH is adjusted to 7.2 with acetic acid or TEA.

Unlysed cells, cell debris, mitochondria, and nuclei are pelleted at 20,000 g for 10 mm at 4° The supernatant is removed with a pipette and centrifuged at 144,000 g for 1 hr at 40 to pellet the secretory vesicles. The supernatant is decanted carefully and the pellet is resuspended in either lysis buffer or another buffer containing osmotic support.

### Example 51

Use of Anti-Sense Oligonucleotides to Inactivate ITIMS In Vitro & In Vivo

The antisense methodologies produce inhibition of specific gene products by exploiting hybridization of complementary nucleic acids, resulting in decreased mRNA stability, or through a block in mRNA processing, transport or translation. An RNA or single stranded DNA that is complementary to the mRNA of ITIM is introduced into immunocytes in vitro. The antisense molecule can form base-pairs with the mRNA, thus preventing translation of the mRNA into protein. Antisense inhibition can be produced either by the transfection of plasmids or by the addition of small single-stranded oligonucleotides. Modifications of these strategies, include inducible antisense vectors, antisense retroviral vectors, and a variety of oligonucleotide modifications to facilitate delivery and enhance efficacy.

Expression vectors are constructed to produce high levels of antisense RNA in transfected cells. Antisense nucleic acids can be introduced into immnunocytes using synthetic single-stranded DNA oligonucleotices. When short oligonucleotides complementary to the sequence around the translational initiation site (the AUG codon) of an RNA in the cells, they hybridize to the mRNA and prevent initiation of translation. Chemically modifying the oligonucleotides can greatly increase the efficiency with which they enter cells and their stability once inside.

Principle of Method

Genetically engineered plasmid-based antisense methods expression vectors which generate RNAs containing sequences complementary to key regions of specific genes are used. These antisense expression vectors prokaryotic and eukaryotic selectable markers allowing vector construction and identification of transfectants, a gene promoter which controls the expression of the antisense RNA, an antisense sequence which is complementary to a bindable region within the specific target gene sequence (5′ untranslated and/or translation initiation regions are often employed as target sites); and a RNA stabilizing sequences to assure stability of antisense RNAs. Optimal gene replacement protocols would include both inhibition of the endogenous gene and overexpression of the preferred (or mutant) gene. Oligonucleotide-based antisense methods use synthetic single-stranded oligonucleotides which may range from simple deoxyribonucleotides to more complex molecules containing base modifications and/or covalent modifications which enhance delivery, uptake, or antisense effect.

Materials and Reagents

Antisense expression vectors are obtained from the laboratories where they were developed or areconstructed by combining the key elements described earlier. These include neomycin as a selectable marker so transfectants are isolated employing Dulbecco's modified Eagle's medium with 10% calf serum and the appropriate concentration of the antibiotic G418 (Bethesda Research Laboratories. Bethesda, Md.). G418 is used for selection of stable transformants.

Pure oligonucleotide preparations and nuclease-free culture conditions are utilized. Antisense oligonucleotides are synthesized by solid state methods and purified by chromatography or gel purification (or obtained from commercial sources). Following purification, the oligonucleotides are lyophilized two to five times in sterile distilled water to remove volatile components. Both unmodified and modified oligonuclcotides are suspended in 10 mM HEPES (N-2-hydroxyethylpiperazine-N′-2-ethanesulfonic acid)-buffered saline at pH 7.4. The pH of these oligonucleotide solutions is checked because the addition of even small amounts of solutions with nonphysiologic pH can have profoundly toxic effects on cells. Nuclease-free culture conditions are employed.

Design and Construction of Antisense Expression Vectors

For inhibition of ITIM expression in mammalian cells a prototype antisense expression vector is used. This vector allows cloning of antisense and control “sense” gene fragments between the metal-inducible metaliothionein promoter and b-globin sequences which are added to provide stability to the antisense sequences. We have also constructed vectors which differ only in the promoter region because the promoter region in these vectors is readily excised by 5′ EcoRI and 3′ HindIII sites: one series of vectors is regulated by the steroid-inducible mouse mammary tumor virus (MMTV) promoter. and another series of vectors is regulated by the constitutive Rous sarcoma virus long terminal repeat (RSV-LTR). Antisense retroviral vectors are constructed which are compatible with our plasmid expression vectors: the promoter cartridge is flanked by unique 5′ XhoI and 3′ HindIII sites, and the antisense cartridge is flanked by unique 5′ HindIII and 3′ BamHI sites. Because the target sequence for the 84-bp antisense vector resides entirely within the 5′ -untranslated region, this antisense vector inhibits the endogenous ITIM gene. Antisense-resistant expression vectors are constructed by cloning the HaeII-EcoRI fragment of human ITIM cDNA into a Moloney LTR-regulated expression vector removing sequences complementary to the 84-bp antisense ITIM construct. In order to demonstrate which domains of the ITIM gene contribute to cellular inhibition, ITIM C-terminal deletion mutants are constructed by linker insertion of an in-frame termination codon. All of these mutant ITIM plasmids are subcloned into expression vectors for expression in cultured fibroblasts (Moloney-LTR promoter) or F9 embryonal carcinoma cells (Rous sarcoma virus LTR promoter).

Production of Stable Transformants Expressing Antisense RNA

Stable transformants expressing both the antisense-resistant vector and the anti-ITIM RNA construct are obtained by cotransfection of both plasmids using the following protocol which generally results in 20-50 transformants per 20-50 mm² tissue culture dish.

Day 1. 1.2 million cultured mouse fibroblasts (NIH 3T3, BALBc/3T3) are plated per 100-mm² tissue culture plates in 10 ml of Dulbecco's minimal Eagle's medium supplemented with 10% calf serum and allowed cells to attach overnight.

Day 2. Each plate is transfected by calcium-DNA coprecipitation with 20 mg of cesium-banded plasmid DNA. For cotransfection studies, use 2 mg of the antisense vector containing the Neo selectable marker and 18 mg of the antisense-resistant expression vector which lacks this selectable marker. This results in expression of both plasmids in most stable transformants. A DNA-calcium solution is prepared by mixing in the following order: 20 mg of plasmid DNA dissolved in TE (10 mM Tris, pH 7.5. 1 mM EDTA), 50 ml of 2.5 M CaCl₂ and TE to make 500 ml total. This solution is added slowly to 500 ml of 40 mM HEPES-buffered saline (pH 7.2) and is then mixed by bubble aeration to form an opalescent solution. After a 30-mm incubation at 25°, the entire 1-mi coprecipitate is added to the plate. After incubating the DNA coprecipitate with the cells for 4 hr. the media is aspirated, and the cells are treated with 5% glycerol in 20 mM HEPES buffer for 3 mm. After this glycerol shock, the cells are washed again with phosphate-buffered saline (PBS) and then refed with 10 ml of fresh medium containing serum.

Day 3. The media is aspirated and replaced with 10 ml of Dulbecco's minimal Eagle's medium supplemented with 10% calf serum and 1 mg/ml of the antibiotic G418.

Day 5. The medium is aspirated (which by this time contains dead cells and cellular debris from G418-sensitive cells) and refed with 10 ml of Dulbecco's minimal Eagle's medium supplemented with 10% calf serum and 1 mg/ml of the antibiotic G418.

Day 7. The medium is aspirated and refed with 10 ml of Dulbecco's minimal Eagle's medium supplemented with 10% calf serum and 1 mg/mi of the antibiotic G418.

Days 9-12. Once individual clones are clearly apparent they can be isolated with cloning rings and expanded in individual wells of microtiter plates. Multiple clones should be studied for each combination of antisense vector and antisense-resistant rescue plasmid.

Cotransfectant clones are analyzed to quantitate the extent of antisense inhibition of the ITIM gene, the level of expression of the antisense-resistant transfected gene, and the effect of target gene inhibition on cellular reactivity to lipid antigens or superantigens presented alone or in the context of cell bound or soluble MHC or CD1 molecules.

Assays to quantitiate reactivity and analyze antisense inhibition in the presence and absence of antisense RNA are carried out as follows: [³H]Thymidine incorporation of stable transfectants and controls is meaured after exposure to lipid antigens or superantigens in the context of MHC or CD1 either cell bound (or immobilized) or in soluble form. Control “sense” plasmids are similarly treated and tested. In addition, rescue with a wild-type antisense-resistant gene should overcome the antisense inhibition, provide additional assurance that the growth inhibition is actually due to specific inhibition of the ITIM gene.

Antisense inhibition can be quantitated by measuring levels of either target gene m RNA or protein. Nuclease protection assays provide an excellent method for quantitation of antisense effect. Cells from the stable transformant clones are placed in DMEM with 0.5% calf serum for 48 hr prior to steroid treatment and/or serum restimulation. RNA is isolated from cells by the euanidinium thiocyanate method and the total RNA from 2×10⁶ cells is hybridized with 3×10⁶ cpm of the labeled RNA probe for 16 hr at 45°.

Samples are then treated with 2 mg/ml of RNase A and 4 U/ml of RNase T1 for 30 mm at 25° and are deproteinized by sequential treatment with proteinase K and phenol-chloroform followed by ethanol precipitation. and electrophoresis on an 8% denaturing polyacrylamide gel.

Antisense Inhibition with Anti-ITIM Oligonucleotides

Oligonucicotides target regions of the ITIM mRNA which are available for hybridization i.e., a region that is unbound by protein and free of secondary structure. Sequences within the 5′-untranslated region or translation initiation region are employed for design of antisense oligonucleotides. The length of oligonucleotides employed for antisense inhibition varies between 12 and 20 nucleotides. A melting temperature (Tm) of 50-55° is optimal for specific inhibition of target genes. Tm for short oligonucleotides is- best determined by a formula in which G and C residues equal 40 and A and T residues equal 20. Optimal ITIM antisense oligonucleotides range from 14 to 19 nucleotides.

Determination of Oligonucleotide Stability

Oligonucleotides are 5′ end labeled with polynucleotide kinase and [³²P]ATP and are then added to culture media and serum (plus unlabeled oligonucleotide to achieve a final oligonucleotide concentration of 5 mM: for a 15-mer this is approximately 20 mg/ml). Aliquots are removed periodically and resolved on denaturing 20% acrylamide gels.

Detection of Intracellular Duplexes

To detect intracellular duplex, 20 mg of oligonucleotide is 5′ end labeled oligonucleotides with 20 mCi of [³²P]ATP to achieve a specific activity of 50 million cpm/mg. After incubation for 4 hr. unincorporated oligonucleotides are removed by washing the cells three times with HEPES-buffered saline prewarmed to 37° (to prevent the melting of duplexes). The cells are then lysed in 100 ml of Nonidet P-40 lysis buffer (10 mM Tris, pH 7.5. 10 mM NaCl. 3 mM MgCl2, 0.05% Nonidet P-4t)) containing if 0.5% sodium dodecyl sulfate, 100 mg of proteinase K per ml. and a 10,000-fold excess of unlabeled oligomer (as the carrier). Following phenol/chloroform extraction and ethanol precipitation. a S1 nuclease protection assay is performed at 37° and the products are analyzed on a 20% denaturing acrylamide gel (containing 42 g of urea of 100 ml). To demonstrate that the duplex is intracellular and not an artifact of RNA isolation, an “add-back” control is performed in which the measured amount of cell-associated radioactivity is added with carrier (excess unlabeled oligonucleotide) to a lysate of cells that were previously unexposed to oligonucleotide.

To confirm that antisense oligonucleotide effects are due entirely to target gene inhibition, and not merely toxicity due to unexpected effects of double-stranded RNA, reversal experiments are performed employing an excess of anticomplementary (“sense”) oligonucleotide. If an effect is due to hybridization of antisense oligonucleotide sequences to specific gene target sequences, then addition of an excess of sense oligonucleotide reverses the observed effects on cell growth or gene expression but expression of the antisense rescue gene restores the ITIM function. This provides a useful specificity control for the antisense RNA and suggests that the observed results are due to inhibition of endogenous gene expression.

Oligonucleotide preparations can have toxic effects on cells, thus anti-sense oligonucleotide these experiments employ multiple controls to assure that the observed results are not merely due to toxicity. First, the target gene is shown to be inhibited by analyzing levels of mRNA and/or protein. Secondly, the demonstration that oligonucleotides with sufficient sequence dyshomology are able to prohibit duplex formation and third that the addition of an anticomplementary oligonucleotide can reverse the antisense oligonucleotide effects by hybridization competition.

### Example 52

ITIM Gene Knockout Mice Using Homologous Recombination

Gene Targeting and Generation of Mutant Mice

Nucleic acids encoding the ITIM of the immunocyte inhibitory receptors for cell activation by receptors specific for lipid molecules and superantigens are deleted in vitro by use of homologous recombination using insertional mutagenesis. Mice are generated with gene mutations in any gene for which one has a cDNA or genomic clone. Stable cultured lines of totipotential cells from mouse or embryos embryonic stem (ES) cells are grown and manipulated in tissue culture and on introduction into blastocyst embryos and participate in the development of all tissues of the embryo, including the germ line. ES cells altered during their time in culture (e.g., by addition or alteration of genes) rise to lines of mice that inherit the same genetic alteration. Homologous recombination in mammalian cells is used as well as selection of cells which have undergone homologous recombination using positively selectable marker genes are transfected and inserted into any cloned piece of genomic DNA. Negatively selectable markers are applied to one end of the transfected piece of DNA, allowing selection against random insertions in parallel with the selection for the positively selectable marker. With these and other technical advances it is now feasible to mutate the ITIM cloned gene by site-specific mutagenesis in ES cells and thereby generate a mouse line with the desired mutation and functional deficiency.

Gene Targeting Vectors

Homologous recombination frequency is higher, when the gene targeting vector is isogenic with the ES cells and also increases markedly with increasing length of homology. With this in mind, genomic clones are isolated that contain 3-5 kilobases (kb) of sequence homology on each side of the intended ITIM mutation except when the polymerase chain reaction (PCR) is used to screen for homologous recombination. To make an ITIM knockout or null mutation, a deletion is made in the ITIM gene, followed by insertion of a positive selectable marker. Insertion alone is more likely to allow some expression of the gene by aberrant transcription and/or translation. The ITIM mutation is made near the 5′ end of the gene, including deletion of some mature protein sequence. An effective strategy is to delete the ATG, and the signal sequence, if they fall in a single exon. If the start site for transcription is known, this is also be deleted. Such deletion of transcription, translation, and secretion control elements increases the chance of completely ablating expression of the desired protein. The positive selection marker is then inserted in place of the deleted segment.

The positive selectable markers used confer resistance to G418 or to hygromycin B by replacing the bacterial control sequences of the neomycin resistance (neo) or hygromycin resistance (hyg) genes, respectively, with eukaryotic control sequences. Both markers are expressed at many loci in ES cells when controlled by the mouse phosphoglycerate kinase (PGK) promoter and polyadenylation signal. The engineered pMCI promoter the gene encoding hypoxanthine-guanine phosphoribosyltransferase (HG PRT) 21 are also used successfully in ES cells for gene targeting experiments

To enrich for targeted versus random integration of the targeting vector, a positive-negative selection procedure is used which involves addition of a negative selectable marker at one or both ends of the targeting vector. The negative marker is lost during homologous recombination but retained during random integration of the targeting vector. The negative selectable marker utilized is the herpes simplex virus thymidine kinase gene (HSV-TK), which renders cells sensitive to ganciclovir. The HSV-TK gene works well under the control of either the pMCI promoter or the PGK promoter. The same promoter are used for both selectable markers. To minimize random breaks between the positive and negative markers, the distance between the positive and negative markers is less than 5 kb. The targeting vector has a unique restriction site to linearize before electroporation. Several vectors which have eight-cutter sites (e.g., NotI) in their polylinkers are used for the constructions. Screening for targeted cells by genomic Southern blotting requires a probe that is outside the targeting vector. Screening for targeted cells by PCR is also feasible.

Specific Methodology

Embryonic Stem Cells (ESC)

The ES cell lines: CC1.2, CCE, D32 ABI, and JI are useful and are tested for germ line transmission by implanting them into blastocysts of a different coat color genotype. ES cell lines used are from the wild type (agouti) in color129/Sv strain. Injection into C57BL/6 recipient blastocysts produces chimeric mice with a mix of black and agouti (brown) coat color. If the ES cells contribute to the germ line, one expects agouti progeny because agouti is dominant over black.

Embryonic stem cells are used at low passage numbers to maintain totipotency and avoid differentiatiation and are maintained on embryonic fibroblast feeder cells and/or in the presence of a growth factor known as LIF (leukemia inhibitory factor) or both. These feeders are prepared from an appropriate transgenic mouse line to contain genes for resistance to the positive selection drug (e.g., G418/neomycin, hygromycin).

Reagents for Embryonic Stem Cell Culture

ES cell medium: One package of Dulbecco's modified Eagle's medium (DMEM) powder [with glucose (4500 mg/liter), L-glutamine, and sodium pyruvate (Cat. No. 56-499; JRH Biosciences, Lenexa, Kans.)], 134.8 g, is made up to 10 liters, by adding 12.0 g of NaHCO3 and 62.4 g of N-2-hydroxyethylpiperazine-N′-2-ethanesulfonic acid (HEPES). Adjust the pH to 7.5 with 10 N NaOH prior to filtration. This is stable at 40 for at least 3 months. Supplement with fresh glutamine (single-use aliquots of box stock from GIBCO-BRL, Gaithersburg, Md.) after 2 weeks. Once the following components have been added, use the medium within 2 weeks: 360 ml of medium, 65 ml of highest quality fetal bovine serum [FBS; final concentration, 15% (v/v)], 4.5 ml of nonessential amino acids (100× stock from GIBCO-BRL), 3 ml of 14.2M 2-mercaptoethanol, 45 ml (final concentration, 1000 U/mi) of LIF (ESGRO; GIBCO-BRL)

Feeder cell medium: ES cell medium without extra components plus 10% (v/v) fetal bovine serum (quality is less important)

Phosphate-buffered saline (PBS): 0.8% (w/v) NaCI, 0.02% (w/v) KCI, 0.02% (w/v) KH2PO4, 0.115% (w/v) Na,HPO4 (pH 7.4)

Ethylenediaminetetraacetic acid (EDTA): For 1 liter use 100 ml of 10×PBS, 0.2 g of EDTA-Na, 15 mg of phenol red (sodium salt), pH 7.2

Trypsin-EDTA: 40 ml of EDTA stock [0.02% (v/v) EDTA in PBS plus phenol red] plus 1 ml of 2.5% (w/v) trypsin (GIBCO); make fresh after 1 week

HBS: 25 mM HEPES, 134 mM NaCI, 5 mM KCI, 0.7 mM Na₂P0₄, pH 7.1

Preparation of Mouse Embryonic Fibroblast Feeder Layers

Preparation of Primary Mouse Fibroblasts. For G418-resistant feeders, a transgenic mouse line containing a neomycin resistance gene (i.e., any knockout mouse line) is used. Similarly, if hygromycin is used for selection, feeder cells with the hyg gene is prepared.

1. A 14-day pregnant mouse is killed by cervical dislocation, and 70% ethanol is liberally applied to the abdomen.

2. Using a pair of scissors, cut the skin and body wall from genital area to front paws. Move the guts aside to expose the two uterine horns. The embryos will appear as bead-like bulges along the length of each uterine horn. Dissect the uterine horns by cutting below the ovaries, along the mesometrium and at the cervix. Rinse in sterile PBS.

3. Cut the uterus between the embryos to separate and rinse each embryo in fresh PBS. Dissect one embryo at a time, using two pairs of watchmaker's forceps: remove fetal membranes and placenta, pinch off the head, remove the soft tissue (liver, heart, anything dark in color), and rinse the remaining embryo in fresh PBS.

4. Place each embryo in a separate petri dish and transfer, covered, to a tissue culture hood for trypsinization. Aspirate PBS and mince tissue with two scalpels or a fine curved pair of scissors. Add 2 ml of trypsin-EDTA (fresh) to each dish and incubate at 37° for 5 mm.

5. Add 8 ml of feeder cell medium to each dish and allow to settle in a conical tube for 2 mm.

6. Plate out each supernatant in one 10-cm tissue culture dish. Feed with fresh medium after 24 hr. Culture until confluent (about 1-2 days) in a standard tissue culture incubator.

7. Divide each dish into ten 10-cm dishes and culture until confluent (2 days).

8. Trypsinize and resuspend each dish in 1 ml of ice-cold freezing medium [feeder cell medium, 20% (v/v) fetal bovine serum (FBS), 10% (vlv) dimethyl sulfoxide (DMSO) 1.

9. Freeze 1 ml per vial and store at −80° for 24 hr. For long-term storage, store at −135° or in liquid nitrogen.

Feeder Layers for Embryonic Stem Cells

Mitotically inactivated (3,000 rads g irradiation) embryonic fibroblasts at 2.5×10⁶ cells/10-cm dish are used. To achieve a uniform monolayer of feeders, coat plates with 0.1% (w/v) gelatin (sterile) for at least 5 mm prior to plating and aspirate off. Feeders are either pre- or coplated with ES cells. If coplating, or if no medium change is planned before addition of ES cells, spin feeders out of DMSO, as the ES cells are sensitive to it.

Routine Culture of Mouse Embryonic Stem Cells

ES cells are cultured with a feeder layer. Mouse embryonic fibroblast feeders, at a density of 2.5×10⁶/10-cm plate, are suitable for up to 10 days in culture. ES cells are plated at relatively high density, 3×10⁶ to 1×10⁷/10-cm dish. They grow rapidly, and divide every 18-24 hr. ES cell culture are fed daily with ES cell medium. The cells need to be passaged about every third day, and require a second feeding on the last day before passage. Generally, higher viability is achieved if the cells are refed 2-3 hr prior to any trypsinization (for either passage or freezing).

Passaging Embryonic Stem Cells

1. The medium is aspirateand and each plate rinsed once with the same volume of prewarmed PBS.

2. For a 10-cm plate, 2 ml of prewarmed trypsin-EDTA is added and the plate is placed in an incubator for 5 min. until cells detach on swirling the plate.

3. Trypsin activity is stopped by adding 2 ml of ES medium.

4. Cells are dispersed thoroughly with a Pasteur pipette and transfered to a conical tube with an additional 6 ml of medium.

5. The cells are spun for 2 mm at 500 rpm. This spinout of trypsin is important, as ES cells do not grow well in the presence of trypsin.

6. The supernatant is removed by aspiration and replaced with 2 ml of fresh ES medium. Disperse the cells in this small volume first, by pipetting gently (to avoid bubbles, etc.) with a Pasteur pipette 20 times. Thorough dispersal of ES cells is important, as aggregates are more likely to differentiate.

7. The usual split is 1:6, but a more dense plate is often appropriate, for example, a low-density plate that has not been passaged in 3 days. The reason for the split at this time, even though the plate is not confluent, is that the colonies need to be dispersed to prevent differentiation.

Freezing and Thawing Embryonic Stem Cells

The ES are refed 2-3 hr ahead and trypsinized as usual until resuspension. They are frozen at high density; one confluent 10-cm dish can be frozen as six 1-mi vials. ES cells are especially sensitive to DMSO toxicity, therefore exposure is minimized by resuspending cells (with thorough dispersal) in ice-cold medium without DMSO first, and then add an equal volume of 2× freezing medium. For example, for one confluent 10-cm dish of ES cells:

1. The pellet is resuspended in 3 ml of ice-cold ES medium with 20% (v/v) FBS.

2. Ice-cold ES medium (3 ml) with 20% (v/v) FBS, 20% (v/v) DMSO [final concentration, 20% (v/v) FBS, 10% (v/v) DMSO] is added.

3 The samples are kept on ice, and aliquoted to six cryovials.

Vials are frozen slowly in a Styrofoam container for 24 hr at −80°, then store at either −135° or in liquid nitrogen for long-term storage. To use, ES cells are thawed rapidly in a 37° water bath and quickly transferred to ice. The cells are spun out of DMSO in about 10 ml of ES medium, resuspended with thorough dispersal as for passaging and plated on feeders.

Transfection of Embryonic Stem Cells by Electroporation

1. Linear DNA is prepared by phenol-chloroform extraction and ethanol precipitation. Pellet is washed twice in 70% (v/v) ethanol at room temperature and vacuum dried. DNA is resuspended in sterile HBS and considered sterile for use in tissue culture. An aliquot of the preparation ischecked for degradation by comparing restriction maps of linearized and intact plasmid DNA with multiple restriction enzymes.

2. Cells are split 1:6 as usual, 1 or 2 days prior to electroporation. Cells are fed daily and agains 2-3 hr prior to electroporation.

3. Cells are trypsintzed as usual but, on dispersal of cells, add about 10 ml of medium and return the plates to the incubator for 30 mm. This preplating allows about 90% of feeder cells to reattach and thus the cell suspension is further enriched for ES cells. Wash The cells are wasjed twice in HBS and an aliquot of cells is counted.

4. The cells are resuspended in ice-cold HEPES-buffered saline (HBS) at 2.5×10⁷ cells/ml. Linearized DNA is added from a concentrated stock in sterile HBS to a final concentration of 25 mg/ml. and placed on ice for 10 mnin. After mixing with a Pasteur pipette, cells plus DNA are transferred into an electroporation cuvette and kept on ice for 10 min. The 0.4-cm disposable cuvettes from Bio-Rad (Richmond, Va.) and the Bio-Rad Gene Pulser are used for electropotation. Each electroporation cuvette can hold 0.8 ml, equivalent to 2×10⁷ cells.

5. Electroporate at 240 V, 500 mF with the Gene Pulser (Bio-Rad) with a capacitance extender.

6. The cells are allowed to rest for 10 mm at room temperature. Plate The ES cells are plated with, neo-resistant feeders (but no selection drugs) at about 5-7×10⁶ ES cells/10-cm plate.

Selection of Targeted Clones

One to 1.5 days after electroporation, the cells are fed daily with selection drugs until selection is complete (usually 7-8 days). Cell death is observed by 3-4 days after drug addition.

Selection Medium: Dissolve (in 0.1 M HEPES, pH 7.2) an appropriate weight ofG418 powder (Geneticin; GIBCO-BRL) to yield 200 mg of active drug per milliliter if PGK-neo is in the targeting construct or to yield 150 mg of active drug per milliliter, if pMCI-neo is in the construct. Filter sterilize before addition to complete the ES cell medium. For double selection, add ganciclovir (Syntex Corp., Palo Alto, Calif.) to a 2 mM final concentration by making a fresh 1000×(2 mM) stock. Dissolve 5.1 mg of ganciclovir powder in 10 ml of 0.1 M HEPES, pH 7.2. Filter sterilize and add to complete G418 medium to give a final concentration of 2 mM. An alternative to gancyclovir is FIAU (Bristol Meyers Squibb, Wallingford, Conn.), which should be used at 0.2 mM. We routinely prepare the selection medium for a whole experiment and store at 40 for up to 2 weeks.

A Culture medium is changed to selection medium after 24-36 hr. One or two plates are grown in G418 medium only, and are used to calculate the number of neomycin-resistant colonies. Feed the plates every day. Cell death is visible at about 3-4 days of selection. The morphology of the colonies should be observed carefully. Some large colonies start flattening. Clones are picked routinely at day 7 or 8 of selection before they start differentiating (but flattened colonies may still contain ES cells and could be picked). . In the plates containing G418 only, the selection takes slightly longer. Plates are stained with Giemsa, and colonies counted on day 10 of selection.

Other Selection Measures

The procedures given above are suitable for the most commonly used schemes involving the neomycin resistance gene as the positive selection marker and the HSV thymidine kinase gene as the negative selection marker. If HGPRT or hygromycin or puromycin resistance genes are used, the feeders are modified accordingly. One can either obtain a line of mice with the appropriate transgene or use a feeder cell line just during the time of selection. A line of STO cells resistant to both neomycin (G418) and hygromycin can also be used. Embryonic stem cells can, and probably should, be returned to fibroblast feeders at the time of picking the clones. Selection for HGPRT is in standard medium plus HAT (0.1 mM hypoxanthine, 0.8 mM aminopterin, and 20 mM thymidine). Hygromycin selection ranges from 100 to 150 mg/ml and should be tested to determine the effective dose for the ES cells.

Picking Drug-Resistant Embryonic Stem Cell Colonies

Equipment Required:

Microscope in a dissecting hood

Sterile Pasteur pipettes that have been drawn out over flame

Mouthpiece, with tubing and a disposable disk-type filter (0.45-0.8 mm pore size)

Twelve-channel pipettor

Tissue culture plates (96 and 24 well)

Pipette tips (presterllized) in racks that fit 96-well plates

Pipette tips in racks for 24-well plates: These are assembled by placing tips in alternating columns of a 96-well rack

Clustered freezing tubes in 96-well plate format (Costar; Cambridge, Mass.)

Summary of Procedure

Clones are usually picked after 7 or 8 days of selection. Each clone is picked into an individual well of a 96-well plate that contains trypsin-EDTA. This is best performed in a dissecting hood. Each trypsinized colony is then split into a single well of duplicate 24-well plates, so that 1 plate can be used for freezing the colonies, and the other for DNA extraction for Southern blot or PCR analysis. The transfer of cells between 96-well plates and 24-well plates is accomplished with a 12-channel pipettor, which has pipette tips in alternating channels. This allows the transfer of clones 1, 3, 5, 7, 9, and 11 from a 96-well plate into wells 1, 2, 3,4, 5, and 6 of a 24-well plate.

Trypsinization of Selected Clones

1. Selection plates are fed 2-3 hr before picking colonies. The number of colonies to be picked is estimated, and 24-well plates with mouse embryonic feeder cells (2×10⁶ feeder cells/plate, i.e., about 10⁵/well with 0.5 ml of complete medium) are prepared. Each colony is split in half, into wells of duplicate 24-well plates

2. A selection plate is washed once with sterile PBS at 37° and replaced with 10 ml of the same.

3. 60 ml/well of trypsin-EDTA at 37° is placed in 1 row of a 96-well plate Colonies are picked in groups of 12 (one row), and the trypsin-EDTA is maintained at 37°. Colonies should be picked fairly rapidly, so that a group of 12 takes about 5 mm.

4. Individual clones are picked by tearing each away from the surrounding feeder cells, using a ripping/tearing motion, and drawing each gently into the pipette. Before approaching a colony, partially fill a Pasteur pipette with PBS from the plate to prevent sticking of the colony to the glass The volume of PBS aspirated should be small, but enough to prevent the colony from sticking.

5. Each clone is transferred to a well of the 96-well plate containing trypsin-EDTA.

6. Six pipette tips are connected to the multichannel pipettor (channels 1, 3, 5, 7, 9, and 11) and clones 1, 3, 5, 7,9, and 11 are dispersed by pipetting up and down several times with the multipipettor. Transfer 30 ml prepare of each cell suspension to the first row of one 24-well plate (prepared above) and the remaining 30 ml to the first row of the duplicate 24-well plate. Then disperse clones 2, 4, 6, 8, 10, and 12 in the same manner and split then into the second row of each 24-well plate.

7. Alternatively, the trypsin is stopped after picking 12 colonies, in the first row of the 96-well plate, by adding 60 ml of complete medium and dispersing the cells. One can then proceed to picking the next 12 colonies into the second row. This is most helpful when the selection plates contain more than 12 colonies.

8. The cells are fed as soon as possible (e.g.,the next morning is soon enough) as they do not grow as well with trypsin present. Cells are fed daily, with 0.5 ml of complete medium.

Freezing Expanded Colonies

1. One of the duplicate 24-well plates for freezing is used. Cells are frozen when many of the wells are subconfluent, even if some clones are still sparse. This is important to ensure that the majority of the colonies have not grown too large and begun to differentiate. Cells are frozen, at this stage only. in 10% (v/v) DMSO, fetal calf serum, and trypsin-EDTA. For this relatively short-term storage, the trypsin-EDTA does not appear to harm the cells.

2. The cells are fed 2-3 hr before freezing.

3. Place 120 ml of ice-cold 20% (v/v) DMSO80% (v/v) FCS in each Costar cluster tube and maintain on ice. [

4. The cells are washed with warm PBS and trypsinize with 60 ml of pre-warmed trypsin-EDTA for 5 mm at 37°

5. The plate are moved to ice and add 180 ml of ice-cold ECS to each well. The cells are dispersed by pipetting up and down with six tips on the multichannel pipettor. Make Two frozen vials of each clone are prepared by transferring 120 ml of the suspension (clones 1-6) into one cluster tube (tubes 1, 3, 5, 7, 9, 11) and the remaining 120 ml into the neighboring tube in the same row (tubes 2, 4, 6, 8, 10, and 12). Thus, each row of clustered tubes corresponds to each row of the 24-well plates.

6. To freeze The cells slowly, wrap the cluster tube rack in paper towels, place at −20° for about 1 hr, then transfer to −70°. The cluster tube racks are maintained at −70° until positive clones are identified.

DNA Isolation for Genomic Southern Blot Analysis

1. Clones are grown until most are confluent. High-quality DNA can be extracted even from overgrown clones.

2. Wells are washed with PBS, 200 ml of lysis buffer are added to each well, and incubated at 37° for 30 mm. Plates are stored frozen at this point.

Lysis buffer: 150 mM NaCl, 20 mM Tris (pH 7.5), 5 mM EDTA, 0.5% (w/v) sodium dodecyl sulfate (SDS), proteinase K (0.25 mg/ml) (stored frozen as single-use aliquots at 20 mg/ml; add to buffer just before use).

3. Lysates are transferred to 1.5-ml tubes. Racks are arranged to fit the format of a 24-well plate so that a 12-channel pipettor can be used to process the samples through all the steps.

4. Extract with an equal volume of phenol-chloroform. Vortex, spin, and take out the organic phase using a 12-channel pipettor.

5. Extract with chloroform-isoamyl alcohol.

6. Transfer the aqueous phase to clean tubes and precipitate with 20 ml of 3 M sodium acetate (pH 5.2) and 0.5 ml of ethanol, at −20° for 30 min.

7. The pellets are resuspended in 10 mM Tris-HCl (pH 8.0), 1 mM EDTA (TE). The precipitation is repeated as described above, the pellets are dried and resuspended in TE.

To dissolve DNA, leave at room temperature for several hours or at 65° for 15 min. A confluent well from a 24-well plate yields approximately 10 mg of genomic DNA. One-half or one-third of each sample is used to digest with restriction enzymes. To aid in complete digestion, a relatively large (100 ml) volume is used and allowed to proceed overnight. The digests can then be precipitated or reduced in volume in a Speed-Vac (Savant, Hicksville, N.Y.) before loading on a gel.

Polymerase Chain Reaction Screen of Embryonic Stem Cell Clones

An alternative to the Southern blotting screen described above is to use the PCR, which is done on pools of clones. Characterization of ES cell clones by PCR in pools of 12 (blot analysis of PCR products is usually required) is performed as folows. Clones are maintained in culture during this quick initial screen. Positive pools are rescreened as individual clones by PCR, and subsequently confirmed by genomic blot analysis. A PCR primer is characterized that is within the targeting vector (usually in the positive selection marker) and another in genomic DNA outside the targeting vector. These primers are tested for their lowest detection limit and extent of cross-homology. To ensure reliable amplification in small amounts of genomic DNA, the amplification distance is only about 1 kb. This limits the homology with the locus to be targeted that is included in the targeting vector and thus decreases targeting frequency.

1. Drug-resistant colonies are picked by the procedures described above, except allow colonies to settle overnight in 96-well dishes with feeders. Each colony is split into two 96-well dishes by trypsinization of the attached cells. One dish is for PCR analysis and the other is expanded. Each dish has a feeder cells and 0.2 ml of medium. Using a multi-channel pipettor, routine trypsinization is performed using no more than 50 ml of trypsin-EDTA. After thorough but gentle dispersal of cells, 25 ml is transferred to each 96-well dish with 0.2 ml of medium per well. The cells are still in dilute trypsin but can tolerate it overnight, which is enough time to settle. Refeed after 12 hr.

2. One to 2 days after the initial split, the cells are trysinized from 1 of the duplicate 96-well dishes as usual, but do not add medium with serum.

3. Samples are combined by row (12 wells/pool) by dispersing in trypsin-EDTA, followed by a rinse with PBS. Microfuge the pools for 3 min. to spin down the cells.

4. All but 5 ml of supernatant is removed and the pellet resuspended in 50 ml of water and frozen on dry ice.

5. Cells are thawed at 95° for 8 mm and cool. 10 mg of proteinase K (fresh aliquot) is added , incubated at 55° for 30 mm. and incubated at 95° for 8 min. and cooled.

6. Components for a standard 100-ml PCR reaction are added. 20 ml of each PCR reaction is run on an agarose gel and blot (various quick-blot procedures are adequate). Probe with subclone of partial amplification product.

7. The remainder of each clone is expanded by continued culturing until the results of pooled PCR analysis are complete and fed daily.

8. Once the PCR results are in, all clones are individually passed within each positive pool to a 24-well plate with feeders. One-half of each sample is saved for PCR analysis as an individual clone.

9. The cells are fed daily. After 3 days, once the PCR analysis of clones is complete, each positive clone is placed on a 35- or 60-mm plate with feeders to expand for freezing (see below). Feed daily.

Thawing and Expanding Targeted Clones

For each positive clone. thaw one of the cluster tubes rapidly in a 37° water bath, then cool on ice. Do not spin. Plate on a 35-mm plate with feeder cells (4×10⁵ feeder cells per plate) in 2 ml of complete medium. Feed after 12 hr. At this point, cells are cultured in normal medium without G418; it has been reported that cells maintained in G418 have a lower capacity to contribute to the germ line of injected embryos. Depending on the density of each clone, split up to 1:6. Freeze as soon as possible in small aliquots. Freeze enough aliquots for one aliquot per day of blastocyst injection. A subconfluent 60-mm plate can be frozen as eight 250 ml aliquots, in 90% (v/v) FCS and 10% (v/v) DMSO. Such an aliquot can be plated directly after thawing into one or two 35-mm plates with feeder cells, 2 days before injection.

Further Characterization of Targeted Clones

Positive clones detected by the above procedures are retested using several different restriction enzyme digestions to ensure appropriate insertion of the selectable marker into the locus; both flanks of the insertion are checked. To determine if targeted clones have any additional insertions of the targeting vector, DNA is tested with a probe containing the neo gene. Only a single band at the expected size is detected after long exposure. A further simple check of the condition of targeted cells is to count chromosomes:

1. A rapidly growing culture is treated with Colcemid (0.06 mg/ml) for 4 hr.

2. Cells are trypsinized, washed in PBS, and combined with washed cells from culture supernatant.

3. The cells are resuspended in 10 ml of 0.56% (w/v) KCl, incubated at room temperature for 8 min. and spun at 300 rpm for 2 min. at room temperature.

4. All but 100 ml of supernatant is removed and the cells resuspended. 10 ml of fixative [3:1 (v/v), methanol-glacial acetic acid] is added dropwise and incubated for 10 mm.

5. The cells are spun and resuspended in 1 ml of fixative.

6. The cells are dropped, from a height of 10 cm, onto glass slides (2-3 drops per slide, cleaned with ethanol and dried). After the liquid has spread to the edges of each slide, blow dry.

7.After they are completely air dried, stain the slides for 15 min. in 3% (w/v) Giemsa in PBS, wash in water and air dry.

8. To count chromosomes is to photograph the slide on print film so that counted chromosomes can be marked. At least 30 spreads/clone are counted. At least 50% of the spreads have 40 chromosomes. Watch out for spreads with too many chromosomes as well as those lacking chromosomes.

Generation and Breeding of Chimeric Mice

The animal and equipment requirements and procedures for generating chimeric mice by injection of ES cells into blastocysts is as follows. C57BL/6 mice are used as embryo donors and strain CD1 as foster mothers. Contribution of embryonic stem cells, which are derived from strain 129/Sv mice and have agouti coat color, is clearly evident in contrast to the black coat color of the donor embryo.

The following procedure yields healthy ES cells as a single-cell suspension. Two days prior to blastocyst injection, one of the frozen aliquots [90% (v/v) serum, 10% (v/v) DMSO] is thawed rapidly in a 37° water bath and plated directly into two 35-mm dishes with feeder cells (4×10⁵ feeders per plate). The medium is replaced the next morning, daily, and 2 hr before trypsinization. One plate is trypsinized at the beginning of an injection session and the second about 3 hr later, in freshly prepared 0.25% (v/v) trypsin (GIBCO-BRL) 0.02% (v/v) EDTA in PBS at 37° for 5 mm. Cells are gently dispersed after adding an equal volume of ES cell medium and pellet. Cells gently resuspended (by drawing them in and out of a Pasteur pipette 20 times) in 2 ml of ice-cold ES cell medium and kept on ice until transfer to the injection chamber.

General guidelines for blastocyst injection: The procedure requires an inverted microscope with interference-contrast or phase-contrast objectives. The magnification range required is ×40 to ×200. The injection microscope is equipped with a cooling stage (blastocysts are more resilient to the injection procedure when they are kept at 5-10°). A pair of micromanipulators is used one for the holding pipette and one for the injection pipette. The manipulators from Leitz Instruments are especially useful. Flow in each pipette is regulated by hand, using a micrometer to adjust a Hamilton syringe. To obtain donor embryos, a stereo dissecting microscope (total magnification, ×20) is required for flushing blastocysts from the uterus and collecting them for culture.

Blastocysts are cultured in a tissue culture dish, in microdrops of medium under light-weight paraffin oil, in 5% CO2 in air at 37° During an injection session, several small batches of blastocysts are transferred from culture to the injection chamber with trypsinized ES cells. The easiest blastocysts to inject are those that have expanded fully but have not yet hatched from the zona pellucida. Loading ES cells into the injection pipette is done with care so as not to damage the cells. The injection pipette is just large enough to accommodate the ES cells, but not much larger, because damage to the blastocyst would be more likely to occur. Each blastocyst is injected with 15-20 ES cells. After injection, the blastocysts are returned to culture for at least 1 hr before surgical transfer to a pseudopregnant female. The surgery is performed on the bench with general anesthesia, using a stereo dissecting microscope. Each female receives 10-12 blastocysts; uninjected blastocysts are transferred along with injected ones, to help prevent complications in the pregnancy due to small litter size.

Chimeric animals are monitored by coat color. The 129/Sv-derived ES cells give rise to an agouti (brown) coat whereas the recipient C57BL/6 embryos produce black coat color. Desirable chimeras have a high proportion of brown coat. To determine if targeted ES cells have contributed to the germ line of chimeric mice, male chimeras are bred with C57BL/6 females. Agouti pups are the result of ES cell-derived sperm. If the targeted mutation is heterozygous viable, then one-half of the agouti pups will carry the targeted allele. However, if none of the chimeras derived from a given ES cell clone has high coat color chimerism then germ line transmission is unlikely. Discard chimeras if none of the first 60 pups contains the targeted allele and, because most ES clones give chimeras of which around 50% give germ line transmission, we would discard a clone if none of the first 5 or 6 chimeras gives agouti pups.

It is necessary to obtain germ line transmission from at least two independently derived ES cell clones in order to ensure that any phenotype of the resulting mice arises from the targeted mutation and not from some other mutation that occurred during handling of the ES cells.

Genotyping Mice: Tail Blots and Polymerase Chain Reaction Analysis

Genomic DNA, isolated from tail biopsies, is analyzed by either Southern blot or PCR. Generally, mice must be at least 3 weeks old to tolerate the general anesthesia used in cutting the tail. With a fresh razor blade, cut a 1-cm length from the tip of the tail and cauterize the remaining tail with-a soldering iron. DNA is isolated by a simplified procedure that is amenable to large numbers of DNA samples. Transfer the tail biopsy to a 1.5-ml tube that contains 0.5 ml of tail lysis buffer: 100 mM Tris-HCl (pH 8.5), 5 mM EDTA, 0.2% (w/v) SDS. 200 mM NaCl, proteinase K (100 mg/ml) (Boehringer Mannheim, Indianapolis, Ind.). Continuously rotate the samples overnight at 55° and Vortex the tubes, and spin down hairs and tissue debris in a microfuge for 5-10 mm. Transfer the supernatant to a fresh tube containing 0.5 ml of 2-propanol and mix thoroughly. Recover the precipitate with a pipette tip and transfer to a fresh tube containing 100 ml of 10 mM Tris-HCl, 0.1 mM EDTA, pH 7.5. Make certain that DNA is dissolved by incubation at 37° with intermittent vortexing.

To analyze tail DNA by Southern blot, a single-copy probe that hybridizes with wild-type and targeted alleles is required. Generally such a probe has already been characterized for use during screening for targeted ES cells. Generally 15 ml of DNA prepared as above is sufficient for one lane of a Southern blot. Restriction digestion should be carried out in a final volume of at least 50 ml, with at least 40 units of enzyme and in the presence of bovine serum albumin (BSA; 0.1 mg/ml) and 4 mM spermicide. Digestions usually require several hours to overnight for completion. If the DNA does not digest well, a phenol-chloroform extraction followed by reprecipitation is likely to help. A variety of standard blotting procedures suitable for genomic DNA can be used. Capillary blotting to a nylon membrane, ultraviolet (UV) cross-linking, and hybridization in sodium phosphate and SDS at 65° work well.

As an alternative, PCR amplification of unpurified tail DNA using appropriate PCR primers can be used. Tail (5-10 mm) is added to 0.4 ml of PCR tail buffer 150 mM KCI, 10 mM Tris-HCl (pH 8.3), 2.5 mM MgCI2, gelatin (0.1 mg/mI), 0.45% (wlv) Nonidet P-40 (NP-40), 0.45% (w/v) Tween 20]. Incubate overnight at 55° with shaking and with addition of two 25 ml aliquots of proteinase K (10 mg/mI) added at an interval of several hours. Heat at 95° for 10 mm to denature residual proteins, cool to room temperature, and spin. Five microliters of each DNA sample should give definitive ethidium bromide signals after PCR amplification with primers for each allele.

To analyze DNA by PCR, primer pairs are designed that indicate the presence of the wild-type allele versus the targeted allele. This is accomplished as follows: First, both alleles are assayed by a set of three primers, one of which is within the neo gene, as described in Polymerase Chain Reaction Screen of Embryonic Stem Cell Clones, above. Alternatively, separate primer pairs are used to assay the alleles:one pair within the neo gene, and the other pair in the wild-type gene. In both these strategies, designing primers that yield amplification products of different sizes allows detection of both alleles in a single reaction tube and gel lane. Care is taken not to contaminate reagents with amplification products. Organization of reagents into single-use aliquots is highly recommended.

Generation of Homozygotes

To determine if homozygous mutant animals are viable, heterozygous crosses are performed and all pups genotyped at weaning. The expected 1:2:1 ratio of genotypes, or lack of it, becomes evident in three to four litters. However, genotype ratios are kept for all heterozygous crosses performed. Ideally, mice derived from two or three independent targeted clones are available. Heterozygous crosses is carried out for each targeted clone, as well as between clones. The interclonal crosses show that any phenotype is the result of the targeted mutation rather than some other mutation occurring in the ES cell clone.

For a homozygous viable mutation, the next job is to show that the targeted mutation is truly a null allele. Procedures will depend on the gene/protein of interest. This has been performed quite thoroughly for the tenascin-deficient mice and for P-selectin-deficient mice at both the mRNA and protein level.

### Example 53

Animal Models of Infectious Diseases: Testing Superantigen-NTLB Conjugates. Anti-Sense Oligonucleotides & Gene Knockout Mice & Cells Deleted of Inhibitory Receptors for Infectious Disease Associated Antigens (IRLAs) and/or ITIMs or ITAMs Abbreviations: IRLA=Inhibitory Receptor for Infectious Disease Associated Antigens, NTLB=Lipid-Based Non-Tumor (Infectious Disease) Associated Antigens, IRSAs=lnhibitory Receptor for Superantigens, IRLBT=Ihibitory Receptors for Lipid-based Tumor Associated Antigens.

Tuberculosis

Animal Species

C57 BL/6 mice are used. These mice are natural-killer-cell-deficient. Beige mice are infected with many of the nontuberculous mycobacteria: MAC, M. kansasii, M. simiac, M. malmoense and M. genavense. Same-sex mice 5-7 weeks old are allowed to acclimate for 1 week in the facility before being used. They are housed in microisolator units (lab products, Maywood, N.J.) and are randomly distributed six to a group.

Inoculum and Infection Process:

Primary cultures of MAC (M. kansasii or other mycobacteria) to be used for infection are obtained from clinical isolates of patients with disseminated MAC infection, or the American Type Culture Collection (ATCC). ATCC 49602 (serotype 1) strain LPR and MAC 101 (provided by Lowell Young, California Pacific Medical Center Research Institute, San Francisco, Calif.) are used. Organisms are grown in modified 7H10 broth (7H10 agar formulation with agar and malachite green omitted), pH 6.6, with 10% (vol./vol.) Middlebrook oleic-acid—albumin-dextrose-catalase (OADC) enrichment (Difco Laboratories, Detroit, Mich.) and 0.05% (vol./vol.) Tween 80 (Sigma, St Louis, Mo.). Broth cultures are started from one transparent, smooth, flat colony (SmT) grown on an agar plate. The culture tube is placed in an orbital shaker and incubated at 37° C. for 3-5 days. Culture suspensions are predorminately (>95%) of the smooth, transparent, and flat (SmT) phenotype which is more virulent and more resistant to antimycobacterial agents than the smooth, domed, opaque (SmD) or rough phenotypes. After incubation, the culture is diluted in 7H10 broth to a concentration of 10 Klett units/ml (Manostat colorimeter, Manostat, New York, N.Y.) or approximately 5×10⁷ cfu/ml. The inoculum is titrated in triplicate on 7H10 agar plates (Difco) supplemented with 5% (vol./vol.). Middlebrook OADC enrichment. Plates are taped with Blenderm® (3M, St Paul, Minn.), incubated for 2-3 weeks at 37° C., and then counted to determine the precise inoculum.

Treatment Schedule and Controls

The preferred model is to expose mice to a very low inoculum of bacilli using an aerosol generation chamber. After uptake of about 50 bacilli in the lungs the infection grows progressively at first and is then curtailed around 20 days. Laboratory strains such as Erdman attain 4-5 logs in the lungs by this time: more virulent strains such as CSU93 (Tennessee outbreak) and strabn W (New York) can grow to between 6 and 7 logs in this time.

The high-dose intravenous models are also employed. Most of the inoculum is taken up by phagocytes in the spleen and liver and only 1-2% can be detected in the lungs. This then grows progressively for 10-15 days in the spleen, and to a lesser extent in the liver, until acquired immunity comes into play, resulting in a “chronic” disease pattern. Thus, the construct under test is given soon after inoculation or after the disease has become chronic. which more closely resembles the human condition as the patient is probably at this stage before diagnosis is first made.

For iv use the inoculum is injected in a (1.2 ml volume using a 0.5 ml syringe with an attached 28 G 0.5 in. needle to deliver a total of approximately 10⁷ cfu/mouse. Each experiment consists of an early control (sacrificed 1 week postinfection at the initiation of therapy) and a late control (sacrificed at the end of therapy) group, neither of which receives any treatment. One treatment group consists of a drug known to have activity (e.g. azithromycin or clarirhromycin). Treatment is started 7 days postinfection and is generally continued for 10 days in succession. In extended therapy experiments, treatment is given daily (Mon-Fri) for 4 or more weeks.

Mice are weighed at the beginning and end of each experiment, and averaged by group. Mice are evaluated daily and changes in appearance or behaviour are noted. In general, infected mice appear outwardly well, and continue to gain weight with these infections. There have been some exceptions where the infected control animals have succumbed to the infection. Untreated mice develop splenomegaly, hepatomegaly (with visible lesions), and enlarged lungs. Enlargement of the spleen, although minimal, is evident at 1 week postinfection (average weight 0.12-0.14g) and continues to increase for the duration of the experiment (average weight 0.65-0.80 g at 18-20 days postinfection). After 1 week of infection, the usual organ cell counts in the lungs of early control mice (4.5 log) is significantly less than that in the spleen (7 log). The late control mice (19 days postinfection) average 6 log and 7.8 log cfu in their lungs and spleens respectively.

At the completion of the experiment (2 days after the end of the treatment phase), mice are euthanized using CO2 inhalation. Their spleens and right lungs are aseptically removed. Spleens are placed in preweighed tubes to determine their weight. Each organ is placed in a grinding assembly containing saline with 0.09% (vol/vol.) Tween 80 and each sample is ground and allowed to sit for 15 mm to allow for settling of aerosols. The tops of the grinding assemblies are removed and an aliquot is removed. Dilutions are made to the appropriate concentrations for plating using tubes containing double-distilled H,O (to promote red blood cell lysis) with 0.09% (vol./vol.) Tween 80.

Spleen weights are used to estimate dilutions for the plating of the homogenate. Large spleens, such as those belonging to members of the late control group, are heavily infected and need to be diluted. The lungs are less infected than the spleens in this model and usually require 10-100 times less dilution. Each organ is plated at three different dilutions on 7H10 agar (Difco) plates containing 5% OADC enrichment. Plates are incubated for 2-3 weeks and counted to determine viable cell counts, which are expressed as counts/organ.

Key Parameters to Monitor Infection and Response to Treatment

Mice show some or all of the following signs of infection: a hunched posture with or without difficulty ambulating, isolation from the rest of the group in their cage, labored breathing, and shivering, lack of eating and/or drinking and diarrhea.

Upon introduction of a new agent, mice are monitored initially and during the first hour for any indications of pain or discomfort, allergic reaction or swelling. During the first 3 days of therapy, the mice are monitored at least twice daily to note their general appearance. Any injection site is inspected for swelling or irritation. The general condition of the animals is carefully noted. Mice having an acute reaction are euthanized immediately and the therapy should be reevaluated.

Therapeutic Regimen and Treatment Schedule

Therapy is begun 7 days postinfection. Determination of the spleen and lung viable cell counts is done several days after the completion of therapy. Although daily treatment for 10 days allows for differentiation of relative activities, longer treatment periods (4-12 weeks) are useful to characterize efficacy.

The therapeutic constructs are started on day 20 (when the first becomes DTH-positive) and continued 2-3 time weekly for four weeks. Bacterial loads are determined at 35 and 50 days. Isoniazid (25 mg/kg/day) is given as the positive control in each assay.

Outcome

Treated animals show elimination and/or reduction of viable organisms in the spleens and lungs or spleen weights of therapy groups in comparison to those in the spleens and lungs of the control (infected, but untreated) groups. These differences are statistically significant using the Wilcoxin rank test or other statistical method known in the art. Additional comparison is made between the therapy groups and the group given a standard therapy such as clarithromycin or azithromycirl

Leishmaniasis

Animals:

Mice are preferred species and BALB/c is the preferred strain

Infection Procedure

In the mouse model, the intravenous route of infection is used to give the quickest and most reproducible infection in the liver, spleeen and bone marrow.. Prior to infection, mice are warmed in a cage by a light or warm water to raise the tail veins. The inoculum of parasites, either amastigote or promastigotes, is loaded into a 1 ml syringe fitted with a 23G 11/2 needle. For experimental infection, an inoculum of 10⁷ amastigotes in a volume of 0.1 to 0.2 ml is injected intravenously in mice and by the intracardiac route in hamsters. This will produce a microscopically detectable infection in the liver of mice and liver and spleen of hamster after 1 week of infection. This level is suitable for tests of the constructs given herein.

Administration of Therapeutic Constructs

Therapeutic constructs are administered to mice by a variety of routes (s.c., ip. and p.o.) and for some formulations i.v. administration by the tail vein is required. In the mouse model treatment is best evaluated against an established infection on days 7-11 post-infection. If a lower infection inoculum is used, tests can be carried out on days 14-18 post-infection. In the commonly used BALB/c mouse the infection in the liver increases linearly until days 21-28 following infection by 10⁷ amastigotes or promastigotes; after this point the liver infection becomes chronic and eventually cure. The spleen infection, although microscopically detectable from week 1, is fully established after the 4th week of infection. If the infection is left for several weeks prior to treatment, then chronic granulomatous infection is established which has been shown to be less sensitive to standard drugs.

In an alternative approach using the mouse model, treatment with constructs is started immediately after infection. A 5-day course of treatment is sufficient to determine relative potencies of the constructs.

Outcome

In the early stages of infection VL in mice presents no obvious external symptoms. Extra mice or hamsters are infected and sacrificed prior to administering the conjugates to check that the infection is established. Microscopical examination of stained slides prepared from the liver and/or spleen of rodents will indicate whether the inoculum was satisfactory and that infection has been established. The appearance of hamsters does change in the later stages of infection. The most noticeable features are loss in weight and dulling of the hair. Occasionally hamsters may develop ascites. These clinical changes in the untreated controls are contrasted with changes in the treated population. The treated groups show none of these changes in the course of therapy.

At the end of treatment the mice are weighed to give an estimation of drug toxicity. The livers and spleens are removed from freshly sacrificed animals and weighed. Smears are prepared from the livers and spleens on microscope slides, fixed in methanol for 1 minute and stained with Giemsa stain for 45 minutes. The number of parasites/500 liver and/or spleen cells is determined microscopically for each experimental animal. This figure is multiplied by total organ weight (mg) and this figure, the Leishman-Donovan unit (LDU) is used as the basis for calculating the difference in parasite load between treated and untreated animals. Treated animals show complete elimination or reduction in the number of parasites/500 liver and/or spleen cells. The difference in parasite loaded between the treated and control groups is statistically significant using the Wilcoxin rank test. The activity of novel compounds is compared with that of the standard antimonial drugs and expressed as a therapeutic ratio.

Trypanosoma Cruzi

Chagas infection has been observed in different mammal species. Several animal models have been used experimentally such as mice, hamsters, dogs, rats, rabbits and monkeys. The course of the T cruzi infection varies widely between those laboratory animals, depending upon the host and parasite strains used, the route of inoculation and the size of the inoculum.

Mouse Model

Most studies used the mouse model because it is cheaper, easy to work with and it can produce both the acute and chronic phase of the disease. Various mice strains differ markedly in their resistance to T cruzi. More resistant strains might providea good model for the chronic disease. At this stage, the murine model of Chagas' disease is used in experimental therapy. Several strains of mice have been used in this model: Swiss, weight 18-20 g, female; Balb/c, 8-10 weeks, female; albino, weight 18-20 g male.

Preliminary experiments are performed to determine the optimal parasite inocula to insure infection.

The parasites are maintained by serial passage through female C3H/He mice, which is a resistant strain. Mice with parasitemia are bled into heparinized (1000 UA) phosphate-buffered saline (50:50) and cryopreserved.

Hamster Model

Hamsters (non-isogenic Syrian hamsters, Mesocricetus auratus, male/female) are infected with T cruzi. During the acute phase an inflammatory reaction is observed characterized by mononuclear and polymorphous leukocyte infiltration of variable degree in the majority of tissues and organs. In the chronic phase the same kind of lesions can be observed, but the inflammatory process is less severe and characterized by mononuclear infiltration in the myocardium (Ramirezetat., 1994). The authors noted high levels of parasitemia in the beginning of the infection, which varied with the strain used.

Parasite Strain & Stages of Disease

Different parasitic strains behave quite differently in experimental Chagas' disease with regard to characteristics such as the course of infection, the degree of parasitemia, tissue tropisms, histopathological changes and mortality. Several strains of T cruzi have been * used in different animal model and include Y Ernane, Benedito and Vicentina Strains of T cruzi, from different geographical areas, had previously been characterized into various types according to their infectivity rate and tropism in mice. The classification includes the following:

1. Type I, characterized by a rapid course of infection in mice, high levels of parasitemia and mortality around the 9th and 10th day of infection, with predominance of slender forms and macrophage tropism during the acute phase of the infection.

2. Type II shows increasing parasitemia from the 12th to the 20th day of infection, low mortality rate,.predominance of broad forms of the parasite and myocardial tropism.

3. Type III shows a slow development of parasiternia that reaches a high level 20-30 days after inoculation, low mortality and predominance of parasitism in skeletal muscles.

Inoculation & Infection Process

The inoculum range is usually from 1×10³ to 1×10⁷ trypomastigotes (obtained from infected animals) or 2-4×10³ metacyclic trypomastigotes (obtained from triatomid bugs). Acceptable inoculation routes are intraperitoneal and conjunctival. Mice weighing 18-20 g are inoculated by the intraperitoneal route with 5×10⁴-1×10⁵ trypomastigotes which produces a homogeneous infection. Daily trypanosome counts provide the following pattern for the parasitemia: parasites appear from the 4th or 5th day after inoculation, their number decreases markedly on the 6th day, increases until the 7th or 8th day, and decreases again around the 9th day. From the 10th day onwards the pattern of parasitemia is irregular. Most infected animals die in the period from the 5th to the 20th day after inoculation; the highest mortality rates are observed around the 15th day. Mortality rates are about the same for both sexes and only a small number of infected animals will outlive 40 days.

The administration of constructs begins on the day after inoculation and doses corresponding to about one-fifth of the LD50 are given for 10 consecutive days. On the 5 th day after inoculation the number of parasites in 5 mm³ of blood is determined. On the 8th day, when the number of parasites in the inoculated animals is generally higher, a new count is performed.

Outcome

The best initial criteria for therapeutic activity in the experimental Chagas' disease is mortality and parasitemia. Parasitemia is usually high with little variation, depending on the strain used and follow-up is done by daily fresh blood examination. The blood is collected and the parasites counted in a Neubauer's chamber. The acute phase of the infection is followed by a chronic stage in which parasites are reduced to submicroscopic level, then indirect laboratory methods are used, such as subinoculation, xenodiagnosis and serological techniques. The polymerase chain reaction has been used as a complementary criterion for therapeutic activity in the chronic stage of experimental Chagas disease.

The following techniques are used to establish reliable criteria for cure in the mouse model of Chagas' disease:

1. Fresh blood examination: a drop of blood from the mouse's tail is carefully examined in a Neubauer's chamber daily or every other day

2. Blood subinoculation: mice are killed about 1 or 2 months after treatment and 0.4-0.6 ml of citrated blood, collected from the severed axiliary artery, was inoculated intraperitoneally into susceptible mice. From the 5th day of inoculation, fresh blood examinations were performed daily or every other day for a period of at least 6 weeks

3. Blood culture: blood from treated animals was inoculated into Noeller's culture medium and culture was frequently examined for at least 30 days after inoculation

4. Xenodiagnosis: 1 or 2 months after treatment, mice are anesthetized and 4 triatomine nymphae are allowed to feed on them. After 45-50 days, the bugs are carefully examined for trypanosomes

5. Histological examination: histological sections of the heart of treated animals were stained with hematoxylin and eosin and carefully examined.

6. Re-inoculation: some of the treated animals are reinoculated at different after treatment with about 4000 blood parasitic forms per gram of weight; daily counts of trypanosomes are performed so that a new acute phase of the disease might be detected.

Treated animals show 80-100% cures and prolonged survival associated with elimination or reduction in parasitemia compared to untreated control groups. The differences are statistically significant using the Wilcoxan rank test or other statisical methods known in the art.

Anti-Sense Oligonucleotides & IRIDA, ITIM or ITAM Gene Knockout to Delete/Inactivate IRIDAs and/or ITIMs or ITAMS

In a representative ex vivo protocol, using the TB, Leishmania and Trypanosomias models in mice as given above, antisense oligonucleotides 0.01-1 mg corresponding to the coding region of IRIDA ITIM are added to autologous T cells in tissue culture. The uptake or the oligonucleotides by the cells is confirmed using oligonucleotides labeled at the 5′end with fluorescein isothiocyanate (FITC). To check for inactivation of the the inhibitory receptor and ITIMs by the oligonucleotides, Western blot quantitation is carried out on the lysed T cells. The IRIDA genes and their respective ITIM genes are downregulated by >95%. In a parallel experiments, T cell are deleted of the gene encoding the IRIDA genes and their respective ITIM genes by homologous recombination with a mutant gene. The knockout T cells and the anti-sense treated T cells are expanded in IL-2 for 24-72 hours, harvested and reinfused into mice with established tuberculosis, leishmaniasis or trypansomiasis. Optionally the T cells are exposed to LBIDAs for 24 hours before the addition of IL-2. The animals are sacrificed at the end of the experiments and assayed for residual disease as given in the above models of tumreculosis. Results are statistically assessed as given above and show that the adoptively transferred knockout T cells induce a >95% reduction in the number of residual organisms.

In a representative in vivo experiment, mice are inoculated with organisms. The antisense phosphorothioate oligodeoxynucleotide corresponding to the codons for IRIDA genes and their respective ITIM genes and control anti-sense (the same base composition as the antisense with the sequence jumbled) are used. A single dose of ITIM antisense or control antisense (1 mg per 0.1 ml saline per mouse) or saline (0.1 ml per mouse) is injected s.c. into the right flank of mice once the disease is established as in the models given above. At each indicated time, the animals from the control and antisense-treated groups are killed and residual organisms assayed as given in the above models.

The uptake of ITIM antisense in the tissues is carried out by photoaffinity labelling followed by immunoprecipitation of ITIM as follows: The tissues are homogenized with a Teflon/glass homogenizer in ice-cold buffer 10 (Tris-HCI, pH 7.4, 20 mM; NaCl, 100 mM; NP-40, 1%; sodium deoxycholate, 0.5%; MgCl, 5 mM; pepstatin, 0.1 mM; antipain, 0.1 mM; chymostatin, 0.1 mM; leupeptin, 0.2 mM; aprotinin, 0.4 mg ml; and soybean trypsin inhibitor, 0.5 mg ml; filtered through a 0.45-pm pore size membrane), and centrifuged for 5 mm in an Eppendorf microfuge at 4° C. The supernatants are used as tumour extracts. The amount of ITIM in organs is determined by photoaffinity labelling with 8-N3-[³²P]cAMP followed by immunoprecipitation with the ITIM antibodies.

In a second representative in vivo experiment, IRIDA/ITIM knockout mice are prepared by above methods. The are then inoculated with organisms as given in the above models. On day three following injection, the mice are immunized with 0.1-1 mg of LBIDAs alone or conjugated to superantigens (Section 51 and 55) in CFA. If unconjugated LBIDA is given, SEB (0.01-0.1 mg) is administered i.p. 2-3 times per week for three weeks. Separate groups of mice are treated with LBIDAs or superantigens alone or with CFA or saline or without either NTLB or superantigen respectively. The mice are sacrificed on day 21 and the number of residual organisms assayed as given in the above models.

Alternatively, the antisense oligonucleotides complementatry to the IRIDA ITIM coding sequences (0.01-1 mg/mouse) are injected intravenously into mice with established disease as given above. LBIDA and superantigen or conjugates thereof (Sections 51 and 55) are given on the same schedule as immediately above. Infectious burden is assayed at the end of experiments in both treated and untreated groups as given above. Results are evaluated statistically and show >95% reduction in infectious burden in treated animals compared to controls.

### Example 54

Animal Tumor Models: Anti-Sense Oligonucleotides & Gene Knockout Mice and Cells Deleted of Inhibitory Receptors for Lipid-Based Tumor Associated Antigens (IRTAA) and/or Inhibitory Receptors for Superantigens (IRSAG) and/or their ITIMs and/or ITAMs Abbreviations: IRIDA=Inhibitory Receptor for Infectious Disease Associated Antigens, LBIDA=Lipid-Based Infectious Disease Associated Antigens, IRSAG=Inhibitory Receptor for Superantigens, IRTAA=Inhibitory Receptors for Lipid-based Tumor Associated Antigens, LBTAA=Lipid-Based Tumor Associated Antigens

In a representative ex vivo protocol, using the MCA 205/207 murine sarcoma model in C57BL/6 as given in Examples 15-16, antisense oligonucleotide 0.01-1 mg corresponding to the coding region of IRTAA/IRSAG ITIM are added to autologous T cells in tissue culture. The uptake or the oligonucleotides by the cells is confirmed using oligonucleotides labeled at the 5′end with fluorescein isothiocyanate (FITC). To check for inactivation of the the inhibitory receptor and ITIMs by the oligonucleotides, Western blot quantitation is carried out on the lysed T cells. The IRLBT and IRSA genes and their respective ITIM genes are downregulated by >95%. In a parallel experiments, T cell are deleted of the gene encoding their IRLBT or IRSA genes and their respective ITIM genes by homologous recombination with a mutant gene. The knockout T cells and the anti-sense treated T cells are expanded in IL-2 for 24-72 hours, harvested and reinfused into C57BL/6 mice with established pulmonary metastases as described in Example 16. Optionally the T cells are exposed to lipid-based TAAs or superantigen or superantgen transfected tumor cells or superantigen transfected tumor cell/dendritic cell fusion cells (Example 25-26) for 24 hours before the addition of IL-2. The animals are sacrificed 21 days later and the number of pulmonary metastases counted. Results are statistically assessed as given in Examples 15-16 and show that the adoptively transferred knockout T cells induce a >95% reduction in the number of pulmonary metastases.

In a representative in vivo experiment, MCA 205/207 tumor cells (1.5×10⁶) are inoculated s.c. into the left flank of C57/B1 mice. The antisense phosphorothioate oligodeoxynucleotide corresponding to the codons for IRLBT and IRSA genes and their respective ITIM genes and control anti-sense (the same base composition as the antisense with the sequence jumbled) are used. A single dose of ITIM antisense or control antisense (1 mg per 0.1 ml saline per mouse) or saline (0.1 ml per mouse) are injected s.c. into the right flank of mice when tumour size reached 80-100 mg, 1 week after cell inoculation. Tumor volumes are obtained from daily measurement of the longest and shortest diameters and calculation by the formula, 4/3Õr³ where r=(length+width)/4. At each indicated time, two animals from the control and antisense-treated groups are killed, and tumours removed, weighed, immediately frozen in liquid N, and kept frozen at −80° C. until used.

Photoaffinity labelling followed by immunoprecipitation of ITIM is carried out as follows: The tumors are homogenized with a Teflon/glass homogenizer in ice-cold buffer 10 (Tris-HCI, pH 7.4, 20 mM; NaCI, 100 mM; NP-40, 1%; sodium deoxycholate, 0.5%;

MgCl,, 5 mM; pepstatin, 0.1 mM; antipain, 0.1 mM; chymostatin, 0.1 mM; leupeptin, 0.2 mM; aprotinin, 0.4 mg ml; and soybean trypsin inhibitor, 0.5 mg ml; filtered through a 0.45-pm pore size membrane), and centrifuged for 5 mm in an Eppendorf microfuge at 4° C. The supernatants are used as tumour extracts.

The amount of ITIM in tumours is determined by photoaffinity labelling with 8-N3-[³² P]cAMP followed by immunoprecipitation with the ITIM antibodies.

In a representative in vivo experiment, IRTAA/ITIM Knockout C57BL/6 mice are prepared by above methods. The MCA 205/207 tumor cells (2-3×10⁵) are then injected intravenously to induce pulmonary metastases. On day three following injection, they are immunized with 0.1 -1 mg of LBTAAs alone or conjugated to superantigens (Section 51 and 55) in CFA. If unconjugated LBTAA is given, SEB (0.01-0.1 mg) is administered i.p. 2-3 times per week for three weeks. Separate groups of mice are treated with LBTAAs or superantigens alone or with CFA or saline or without either LBTAA or superantigen respectively. The mice are sacrificed on day 21 and the number of metastases in the lung are counted as given in Example 16.

Alternatively, the antisense oligonucleotides complementatry to the IRTAA or IRSAG or ITIM coding sequences (0.01-1 mg/mouse) are injected intravenously into C57BL/6 mice with MCA 205/207 tumors present subcutaneously ((at least 1 mm in diameter) or with pulmonary metastases established by injection of 2-3×10⁵ tumor cells intravenously 3 days before. Lipid-based TAA and superantigen or conjugates thereof (Sections 51 and 55) are given on the same schedule as immediately above. Tumor size is measured weekly in both treated and untreated groups. The number of pulmonary metastases in the treated and untreated groups is determined as given in Examples 16 and 21. Results are evaluated statistically as given in Examples 16 and 21 and show >95% reduction in tumor size and pulmonary metastases in treated animals compared to controls.

### Example 55

Preparation of Lipid-Based Tumor Associated Antigens (LBTAAs) & Lipid-Based Infectious Disease Associated Antigens (LBIDAs) of Bacterial, Fungal, Yeast, Parasitic, Mycobacterial, Invertebrate and Protozoan Origin

Isolation and Characterization of Glycosphingolipids

The following are procedures for obtaining purified glycosphingolipid antigens and lipid-based superantigen receptors (digalactosylceramides) from biological sources for use in treatment of cancer and infectious diseases. The major membrane bound glycosphinolipids useful for treatment of cancer include the alpha mono-, di- and tri- galacotsylceramides in mannalian cells , phytosphingosines present in yeast, vertebrate and plant cells as the well established array of tumor associated membrane antigen e.g., GD2 neuroblastoma associated antigens. It also includes the vast array of glycosphingolipid antigens associated with infectious diseases as given in Section 54.

The basis for extraction of glycosphingolipids from biological sources is their solubility in chloroform-methanol mixtures. Gangliosides (glycosphingolipids containing neuraminic acids) and glycosphingolipids with five or more carbohydrate residues are not only soluble in chloroform-methanol mixtures but also form molecular aggregates that are soluble in water. Glycosphingolipids with one to four residues, form emulsions in water which is the basis for the Folch partition (chloroform-methanol (2:1) with one-fifth volume of water or dilute KCl solution) in which gangliosides are partitioned into the upper water-methanol layer and neutral glycosphingolipids remain in the lower chloroform-methanol layer. Most glycosphingolipids are easily extracted from tissue or other material with chloroform-methanol (2:1) but quantitative extraction of gangliosides requires more polar extraction mixtures such as chloroform-methanol (1:1) or chloroform-methanol (1:2). Metal ions also affect the distribution of gangliosides in biphasic systems. Glycosphingolipids are separated from a total lipid extract by silicic acid column chromatography followed by thin-layer chromatography. Ion exchange cellulose (DEAE) column chromatography is used to separate acidic compounds, such as sulfatide and gangliosides, from less acidic or nonpolar compounds.

Extraction and Partition

All solvents used in the following procedures are redistilled from glass to remove nonvolatile compounds. Chloroform is stabilized by the addition of methanol (after distillation) to a final concentration 0.25% (by volume). Chloroform-methanol and other mixed solvents are given as volume/volume ratios. A weighed portion of the tissue to be extracted is vigorously homogenized with seven volumes of methanol (w/v) in a blender or homogenizer. Fourteen volumes of chloroform are added and the mixture homogenized again. The final solvent ratio is chloroforrm-methanol (2:1). The material is filtered with a Buchner funnel using an aspirator and a coarse-grade solvent-washed filter paper. The residue is reextracted with 10 volumes (based on weight of the original material) of chloroform-methanol (2:1). After filtration, the residue is extracted a third time with 5 volumes (v/v) of chloroform-methanol (1:1) or chloroform-methanol (1:2). The third extraction is only necessary for quantitative removal of gangliosides. The final combined extract is adjusted by addition of chloroform so that the final proportion is chloroform-methanol (2:1). A volume of 0.1 M KCl equivalent to one-fifth that of the final solvent extract is added, mixed vigorously, and allowed to stand at 4° overnight or until the layers are completely separated. If the volumes are small, the layers are separated by centrifugation. The upper and lower layers are washed three times with theoretical lower and upper phases, respectively, prepared by shaking a mixture of 1 volume of chloroform-methanol (2:1) and 0.2 volume of 0.1 M KCl in water and letting the phases separate. With large volumes of combined extracts, the solvents are evaporated in vacuo and the residue redissolved in a convenient volume of chloroform-methanol (2:1) for the partition and washing steps described above.

The combined lower phases (original and washes) are collected and reduced in vacuo to a small volume with gentle warming (<50°) on a rotary evaporator (fraction I). The combined upper aqueous phases are dialyzed against cold tap water for 24 hours and then lyophilyzed (fraction II). The lyophilyzed material, usually containing some insoluble protein, is extracted with chloroform-methanol-water (10:5:1), filtered and reduced to a small volume on a rotary evaporator. Fraction II generally contains only gangliosides and may be analyzed by thin-layer chromatography without further purification (see below).

Silicic Acid Column Chromatography

The lipids from the chloroform-methanol layer (fraction I) are fractionated into neutral lipids, glycolipids, and phospholipids on a column of Unisil silicic acid (Clarkson Chemical Co., Williamsport, Pa.). This procedure is useful for glycolipids containing from one to four glycosyl residues and for sulfatides. Inisil (20-40 g per gram of lipid) is activated at 80° for several hours and is slurried with chloroform as quickly as possible after removal from the oven and poured into a column. The adsorbent is washed with about three bed volumes of chloroform or until it is translucent. The column is not allowed to run dry. A 20 mg/ml solution of the sample is applied in chloroform. Neutral lipids are eluted with about five bed volumes of chloroform and then glycosphingolipids are eluted with 8 to 10 bed volumes of acetone-methanol (9:1). Phospholipids are eluted with 5 bed volumes of methanol.

A different procedure has been used to isolate a particular glycolipid on a preparative scale, as in the purification of trihexosyl ceramide, gal (1®4) gal (1®4) glc-ceramide, from a kidney of a patient with Fabry's disease (trihexosylceramidosis). A crude glycolipid and phospholipid mixture is obtained from fraction I by addition of 200 ml of ether and filtration of the resultant glycolipid-phospholipid precipitate at room temperature. The glycolipid mixture (3 g in one experiment) is then subjected to mild alkali-catalyzed methanolysis. A silicic acid column (400 g) is prepared in chloroform-methanol (19:1), and the sample applied in chloroform-methanol (19:1). The column is eluted successively with 1500 ml each of 12%, 14%, 16%, 20%, 30%, and 50% methanol in chloroform. Fabry trihexosylceramide (1.5 g) is eluted as a pure compound in the 20% fraction. The 16% and 30% fractions also contain some (1 g) of the Fabry lipid mixed with other glycolipids. A similar mixture of glycolipids is fractionated on a silicic acid column using a continuous gradient from 5% to 50% methanol in chloroform.

Mild Alkali-Catalyzed Methanolysis

The glycolipid fraction from the silicic acid column is treated with mild base to remove contaminating phospholipids. This treatment does not affect glycolipids or gangliosides unless they contain an O-acyl group. The following quantities are used for 1-10 mg of glycolipid fraction. Add 1 ml of chloroform and 1 ml of 0.6 N NaOH in methanol to the dry fraction and allow the mixture to react at room temperature for 1 hour. Then add 1.2 ml of 0.5 N HCI in methanol, 1.7 ml of water, and 3.4 ml of chloroform, mix well, centrifuge, and remove the lower layer containing the glycolipids. Wash the lower layer centrifuge, and remove the lower layer containing the glycolipids. Wash the lower layer a three times with methanol:water (1:1) and then evaporate it to dryness in vacuo. If a ganglioside fraction is to be methanolyzed, the sample is treated in the same way except that after neutralization with methanolic HCl the sample is dried in vacuo, emulsified in water and dialyzed against tap water at 4° for 24 hours. The nondialyzable material is lyophilyzed and applied to TLC plates as described below.

Thin-Layer Chromatography

Glycosphingolipids are separated on thin-layer plates of silica gel G, H, or HR. The plates are prepared and activated at 100° C. for 2-4 hours. Plates of 0.25 mm thickness are used for general work and plates of 0.75 mm thickness are used for preparation of large quantities of material. Thin-layer tanks are lined with paper and equilibrated with solvent for 4 or more hours before use. Commercial pre-prepared TLC plates (Quantum Industries, Fairfield, N.J., Brinkman Instruments Inc., Westbury, N.Y., and Analtek Inc., Wilmington, Del.) have been used successfully for qualitative analysis of glycosphingolipids. Separation on these plates, however, is not usually as great as on plates made in the laboratory and contaminants are often obtained when silica gel is removed from pre-prepared plates and eluted with solvents.

A glycolipid mixture obtained from a column is separated into various components on a silica gel H plate (0.25 mm) using a chloroform-methanol-water (100:42:6) solvent system. Some hematoside, NANA(2-3)gal(1-4)glc-ceramide, (FIG. 1, lane A) is usually partitioned into the lower phase of a Folch wash and is separated from human or porcine globoside, gaINAc (1-3) gal (1-4) gal (1-4) glc-ceramide in this system. Monohexosyl ceramide, glc- and gal-ceramide, and dihexosyl ceramide, gal (1-4) glc-ceramide and gal-(1-4) gal-ceramide, often appear as two spots because of the presence of a-hydroxy fatty acids in the ceramide. Otherwise the two forms of monohexosyl and dihexosylceramide are not separated on silica gel alone. Glucosylceramide and galactosylceramide, however, have been resolved on borate-impregnated thin-layer plates. Sulfatide (galactosylcerarnide sulfate) is not usually completely separated from dihexosylceramide, but these compounds can be completely separated by DEAE chromatography. Gangliosides larger than hematoside remain very near the origin in this system.

Gangliosides and neutral glycosphingolipids with more than four glycosyl residues are separated by more polar solvent systems such as chloroform-methanol-water (60:45:10) or chloroform-methanol-2s N NH4OH (65:45:9). In the latter case, when gangliosides are involved, the plate is developed two times with thorough drying (4 hours at room temperature) between developments. Hematoside is well separated from globoside on silica gel G plates with this system.

Glycosphingolipids are visualized with iodine vapor or by spraying with a 2% a-naphthol solution (in ethanol) followed by concentrated H2S04 spray and heating for 10 minutes at 100°. The a-naphthol spray gives deep red-purple spots with carbohydrate-containing compounds and brown spots with phospholipids or neutral lipids. As little as 1-10 mg of material is visualized in this way. Gangliosides are specifically visualized by spraying with the following solution: mix 10 ml of 3% resorcinol (stored in refrigerator) with 80 ml of concentrated HCl, 0.25 ml of 0.1 M CuSO4 and enough water to make 100 ml of solution. The sprayed plate is placed horizontally in a closed jar and heated in an oven at 125° for 20 minutes. Gangliosides appear as black or purple areas and other compounds appear as light brown areas.

Preparative thin-layer chromatography is carried out by streaking the sample on a 0.75 mm thick plate and developing as outlined above. Only the edges of the streak are visualized with I₂ or a-naphthol and areas containing neutral glycolipids are removed from the plate and the silica gel is eluted with chloroform-methanol-water (100:50:10). Gangliosides are eluted from silica gel with more polar solvents such as chloroform-methanol-water (50:50:15).

DEAE Column Chromatography

Water-soluble oligoglycosylceramides are separated from gangliosides from fraction II by the following procedure. Diethylaminoethyl cellulose (DEAE) in the acetate form is washed and columns are prepared. The sample is applied in chloroform-methanol (7:3) and neutral glycolipids are eluted with 8 bed volumes each of chloroform-methanol (7:3) and (1:1). Gangliosides are retained on the column and are eluted with 10 bed volumes of chloroform-methanol (2:1) saturated with aqueous 58% NH₄OH.

Dihexosylceramide and sulfatide isolated from a preparative TLC plate as described earlier is separated on a DEAE column. The sample is applied in chloroform-methanol (9:1) and neutral dihexosylceramide is eluted with 10 bed volumes each of chloroform-methanol (9:1) and chloroform-methanol (7:3). The sulfatide is eluted with chloroform-methanol (4:1) made 10 mM with respect to ammonium acetate, to which is added 20 ml of 28% aqueous anmmonia per liter. Sulfate analysis of lipid fractions is carried out.

Florisil Column Chromatography

An alternative method of isolation of glycosphingolipids is by Florisil column chromatography of the peracetylated glycolipids wherein all the glycophingolipids (except polysialylgangliosides) are isolated in one fraction. Briefly, the procedure consists of peracetylation of the total lipid extract with pyridine-acetic anhydride (3:2) (1 ml per 50 mg of dry total lipid). The pyridine and acetic anhydride are removed in vacuo with additions of toluene, and the products are applied to a Florisil column (40 g per gram of lipid), and neutral lipids and cholesterol are eluted with dichloroethane (8 bed volumes). Peracetylated glycosphingolipids are eluted with 8 bed volumes of dichloroethane-acetone (1:1), and phospholipids are eluted with 5 bed volumes of dichloroethane-methanol-water (2:8:1). Acetyl groups are removed from the glycolipids with 0.25% sodium methoxide in chloroform-methanol (1:1) (1 ml per 25 mg of lipid) at 25° for 30 minutes. The mixture is neutralized with acetic acid, emulsified in water and dialyzed overnight at 40 The glycolipid fraction, analyzed by TLC, is free of contaminating phospholipids.

Characterization of Glycosphingolipids

The first step in the characterization of glycosphingolipids is the complete cleavage of the lipid into its component parts which is carried out by methanolysis with 0.75 N methanolic HCl. The products of methanolysis of a glycosphingolipid are sphingolipid bases and their 0-methyl derivatives, fatty acid methyl esters, and methyl glycosides. These components are separated by solvent extraction and analyzed by gas-liquid chromatography.

Methanolysis

A solution of a glycosphingolipid (up to 1 mg), isolated from columns or thin-layer chromatography plates, is evaporated to dryness in an 8-ml culture tube fitted with a Teflon-lined cap. Three milliliters of 0.75 N methanolic HCl (prepared by bubbling gaseous HCl into methanol) is added to the sample, and the capped tube is heated at 80° for 12-20 hours. At the end of this period, 0.05-0.3 mmole of mannitol (in methanol) is added as an internal standard. The sample is extracted three times with 1 ml hexane to remove fatty acid methyl esters. The hexane solution of methyl esters is retained for GLC analysis. Approximately 100 mg of solid Ag₂CO₂ is added to each tube and carefully mixed until neutral. Methyl glycosides of amino sugars and neuraminyl methyl ester, and sphingosines- are N-acetylated by addition of 1 ml of acetic anhydride. The remaining Ag,C03 and AgGl act as catalyst for this reaction. The mixture is allowed to react for 6-16 hours at room temperature, after which the sample is centrifuged and the precipitate is washed with methanol several times. About 0.25 ml of H₂0 is added to decompose excess acetic anhydride, and the sample is evaporated under a stream of nitrogen. If N-acetylation is not performed, the neutralized sample is centrifuged, washed, and evaporated to dryness under nitrogen.

Trimethylsilylation and Gas-Liquid Chromatography of Methyl Glycosides

Dry samples of methyl glycosides are converted to trimethylsilyl (TMS) derivatives by addition of pyridine.-hexamethyldisilazane-tri-methyichlorosilane (8:2:1) (about 50 mM for 500 mg of lipid). The mixture is allowed to stand for 15 minutes at room temperature and an aliquot is injected into the gas-liquid chromatograph. The mixed silane solution is cloudy and is used without centrifugation, but exposure to water vapor is avoided. If very small amounts of sugars are present, the sample is evaporated under nitrogen and redissolved in a convenient solvent such as pyridine or CS₂.

An aliquot of the solution of TMS derivatives is injected into a gas-liquid chromatographic column (2 m by 3 mm) of 3% SE-30 or OV-1 on Supelcoport (80/100 mesh, Supelco Inc., Bellefonte, Pennsylvania) at 160° with nitrogen carrier gas (25 ml/minute). Programming from 1500 to 2500 at 30 per minute is useful when sialic acids are present. A. chromatogram of a methanolyzed sample of globoside, gaINAc(1-3)gal (1-4) gal (1-4) glc-ceramide shows three peaks for TMS methyl-D-galactoside (α, γ, and β forms); two peaks for methyl-D-glucoside (α and β forms); and two major peaks for methyl-2-acetamido-2-deoxy-D-galactoside. Other methods are useful for gas-liquid chromatography of methyl glycosides are suitable for identification of glycolipids containing fucose, glucose, galactose, galactosamine, glucosamine, and sialic acid. Mannose exhibits peaks overlapping with galactose and if these two sugars are present, the method employing alditol acetates is preferred.

The ratios of glucose and galactose are determined without conversion factors by simply comparing the ratio of the totat peak areas of each methyl glycoside. Since many glycosphingolipids contain only one glucose, ratios are usually expressed in relation to glucose and for globoside the ratio of galactose to glucose is 2. The ratio of galactosamine to glucose calculated in this way is usually about 0.65 for globoside. Methanolysis, N-acetylation, and trimethylsilylation is carried out to obtain reproducible ratios for hexosamines. The mass ratio obtained for N-acetylneuraminic acid to glucose is usually 1.0 to 1.2, and these values should be compared with those obtained from known gangliosides treated in the same way. The absolute quantity of galactose and glucose are determined by comparison to the internal standard mannitol with the use of the following equation.

mmoles glucose=area of glucose peaks

area of mannitol peak×1.25×mmoles of mannitol added

The mannitol peak falls between the second glucose peak and the first galactosamine peak and does not interfere with either compound. The area of peaks is calculated by triangulation.

Fatty Acids and Sphingosines

Normal fatty acids and a-hydroxy fatty acids are determined qualitatively and quantitatively by gas-liquid chromatography of the fatty acid methyl esters obtained from the hexane extract of the methanolyzate. .Sphingosines are determined by hydrolysis of the glycolipid with aqueous HCl followed by N-acetylation and GLC of the TMS derivatives. A colorimetric assayand a method involving GLC of aldehydes produced by NaIO4 cleavage of sphingosine are also available.

Enzymatic Degradation of Glycosphingolipids

Specific glycosidases are used for sequence determination and anomeric analysis of glycolipids. Glycosyl residues are released sequentially from globoside (cytolipin R reacts in the same way) by stepwise treatment with the following glycosidases; b-hexosaminidase from jack bean, a-galactosidase from fig ficin, and b-galactosidase from jack bean. Reactions are carried out with 100 mg of lipid in 0.1 ml of 0.1 M sodium citrate buffer at pH 5, containing 100 mg of crude ox bile sodium taurocholate. After 18 hours at 37°, reaction mixtures are frozen and lyophilized. One milliliter of chloroform-methanol (2:1) is added and the mixture is sonicated for 5 minutes. After centrifugation, the supernatant fraction is dried, taken up in a small amount of chloroform-methanol (2:1) and spotted on a silica gel HR plate. The plate is developed in chloroform-methanopwater (100:42:6) and visualized with 12 vapors or a-naphthol spray. Products are identified by cochromatography with standards and by elution, methanolysis and GLC analysis.

Mass Spectrometry of TMS Glycosphingolipids

Mass spectrometry of intact TMS derivatives of glycolipids gives information about the sugar groups, the fatty acid and the sphingosine portion of glycosphingolipids. Bis (trimethylsilyl)trifiuoroacetamide (100 ml) and pyridine (50 ml) are added to 20-200 mg of the purified glycosphingolipid in a small capped vial and heated at 60° for about 30 minutes. An aliquot containing 10-20 mg of the TMS glycolipid is evaporated to dryness under nitrogen in a mass spectrometer direct probe tube. The samples are volatilized in the mass spectrometer ion source at temperatures ranging from 10° to 180° depending on the size of the oligosaccharide unit.

The following information can be obtained by comparison of the resulting mass spectra with those of reference samples: (1) whether the terminal residue is a hexose or hexosamine; (2) the number of and nature of —N-acetylneuraminic acid groups (i.e., terminal or branched); (3) whether N-acetyl and/or N-glycolylneuraminate is present; (4) information regarding the number of glycosyl residues present and the fatty acid and sphingosine composition. Because of the the inability to distinguish between hexoses, this technique in conjunction with other techniques, such as permethylation analyses, and studies with specific glycosidases.

Ozonolysis of Glycosphingolipids

The carbohydrate portion .of glycosphingolipids is cleaved from the lipid portion. The glucose-sphingosine linkage is broken but there is no hydrolysis of other glycosidic linkages, including those of sialic acid residues. The glycolipid (100 mg) is ozonized in 50 ml of methanol at room temperature. Ozone consumption is monitored by bubbling the effluent gas through a KI-starch solution which blackens when excess ozone is present. The solution is dried in vacuo and the compound is hydrolyzed with 10 ml of 0.2 M Na₂CO₂, for 12 hours at 20°. Sodium ion is removed by stirring with Dowex 50 (H+) and the resin is filtered. After a Folch partition, the upper aqueous phase is lyophilized and the resultant oligosaccharides (about 80% yield) are stored in a desiccator. The procedure is changed for microscale operation (1 mg of lipid).

Permethylation of Glycosphingolipids

Permethylation, hydrolysis, and gas-liquid chromatography of glycosphingolipids is used to determine linkage of glycosyl residues. Permethylation is carried out using methyl sulfinyl carbanion. Sodium hydride (0.88 g of 57% in oil) is washed six times under nitrogen with dry hexane, drained thoroughly, and stirred with dimethyl sulfoxide (10 ml) under a stream of nitrogen at 70° for 3 hours or until bubbling ceases and the solution turns a dark clear green. Any dark precipitate is removed at this point by centrifugation. The carbanion solution (about 0.5 ml) is added under a stream of nitrogen to the glycolipid sample in 0.5 ml of dimethyl sulfoxide, and the mixture is sonicated briefly. After standing at room temperature for 2-6 hours, 1.5 ml of CH3I is added dropwise under nitrogen, and the mixture is allowed to react for 1 hour. After this step, it is not necessary to keep the reaction dry. The permethylated glycolipids are extracted into chloroform, the chloroform layer is washed once with 1% Na2S3O3 to remove I₂, and four times with water. The chloroform fraction is mixed with absolute ethanol and is evaporated under nitrogen. Two milliliters of 1 N H2S04 is added, and after heating at 105° for 12 hours the hydrolyzate is neutralized with BaCO₃ diluted, and filtered on Celite and filter paper, washing the Celite twice with water (5 ml). The sample is concentrated to 5 ml and percolated onto a small Dowex 50 H+ column. Neutral sugars are eluted with water and methanol-water (1:3) (10 ml each) and amino sugars are eluted with 0.3 N NH4OH. The resulting partially methylated sugars are reduced with NaBH4, the products are acetylated and gas-liquid chromatography is carried out.

Partial Hydrolysis of Glycosphingolipids

The presence of N-acetylneuraminic acid or N-glycolylneuraminic acid in a ganglioside sample is determined by mild acid hydrolysis of the neuraminic acid followed by TLC or GLO analysis. The N-acylneuraminic acids are released from gangliosides (0.1-0.2 mg) with 1 ml of 0.05 M HCl (aqueous HCl) at 80° for 1 hour. The solution is extracted with chloroform and the aqueous phase is percolated through about 2 g of Dowex 1 (acetate form) in a small column. The column is washed with 8 ml of water, and the neuraminic acids are eluted with 10 ml of 1 M formic acid. The sample is analyzed on silica gel C plates using n-propanol-water-concentrated ammonia (6:2:1). The Rf of N-acetylneuraminic acid is 0.41 and that of N-glycolylneuraminic acid is 0.28. This sample is also be derivatized with bis (trimethylsilyl) trifloroacetamide and analyzed by GLC. Neuraminic acids are usually analyzed by GLC as their TMS-methyl ketoside methyl ester derivatives. A ganglioside sample (0.1 mg) or mixture of glycolipids is methanolyzed with 2 ml of 0.05 M methanolic HCl. (1 part 12 N HCl and 240 parts methanol) at 80° for 1 hour. The cooled solution is extracted three times with 3 ml hexane and the methanolic layer is evaporated to dryness under nitrogen. Pyridine-hexamethyldisilazane-trimethylchlorosilane (8:4:2) (50 ml) is added and the TMS derivatives are analyzed on a 3% OV-1 column (2 m×2 mm) at 205°. The sulfate moiety of sulfatide (up to 2 mg) is released by reaction with 2 ml of 0.05 N methanolic HCI for 4 hours at room temperature. The reaction mixture is neutralized with aqueous NaOH and the glycolipid product is extracted by Folch partition with 4 ml of chloroform. The chloroform layer is washed with 2 ml of methanol-water (1:1) and is analyzed by TLC.

The carbohydrate sequence of glycosphingolipids is determined by partial degradation under mild acidic conditions. The glycolipid (1 mg) is hydrolyzed with 1 ml of 0.3 N HCl in chloroform-methanol (2:1) at 60° for various times up to 2 hours. After Folch partition (with addition of 0.2 volume of water) the lower phase is dried in vacuo and the upper phase is deionized with Amberlite CG-4B resin (OH form). The glycolipids (from lower phase) are purified by TLC and analyzed by GLC after complete hydrolysis. The upper phase is analyzed for sugars (and polysaccharides) by GLC. Trihexosyl ceramide, gal(1®4)gal (1®4)glc ceramide, hydrolyzed for 2 hours in this way did not yield ceramide but yielded cerebroside containing only glucose, and a dihexosyl ceramide containing glucose and galactose in a 1:1 molar ratio. The water-soluble fraction contained galactose and a disaccharide but no glucose. These data provide evidence for a linear arrangement of the hexose units as given above.

Characterization of the oligosaccharide moieties of glycolipids by partial degradation is also been carried out by treatment of glycolipids with 0.1 N aqueous HCl for 30 minutes to 3 hours, and by treatment of gangliosides with 0.01 N H₂S0₄ at 85° for 1 hour and 0.1 N H₂S0₄ at 100° for 1 hour.”

Mixed Molecular Species of Glycosphingolipids

The purification and characterization of glycosphingolipids is complicated by the fact that naturally occurring glycosphingolipids which are homogeneous in the carbohydrate portion are heterogeneous in the sphingosine and fatty acid portions. Complete hydrolysis and subsequent analyses of the fatty acids and long-chain bases provide information about the extent of this heterogeneity. Additional heterogeneity results when there are glycosphingolipids with identical composition but which differ in glycosidic linkage and/or anomeric configuration of one or more glycosidic bonds. Such mixtures probably cannot be separated by TLC alone. Whether or not products which are homogeneous by TLC actually contain such mixtures can only be determined with techniques such as permethylation and sequential enzymatic degradation.

Extraction of Inositolphosphorylceramides (InsPCers)of Fungi and Yeast

Extraction of lnsPCers from Saccharomyces cerevisiae and from the mycelial phase of Neurospora crassa is as follows: Cells uniformly labeled with [³H]inositol are used to gauge the extraction efficiency of a variety of procedures. Treat the cells with 5% trichloroacetic acid at 0° C. to destroy phospholipases, known to be activated by organic solvents during lipid extraction, followed by extraction with a slightly alkaline, warm, water-rich mixture of ethanol-diethylether-water-pyridine. This method approached 100% extraction of the labeled inositol; Some water-poor solvents completely failed to extract the InsPCers. The adopted procedure has been used for InsPCer extraction from other fungi such as Histoplasma capsulatum and from fresh tobacco leaves and has been used for the extraction of lipophosphoglycan from Leishmania donovani. In the absence of labeling of lnsPCer components, efficacy of InsPCer extraction could be judged by monitoring total long-chain base and/or very-long-chain fatty acids.

Purification of InsPCers

Purification of INsPCers is no different from any very polar, acidic lipid. First, InsPCers precipitate almost quantitatively at low temperature after adjusting to pH 5 the initial lipid extracts from S. cerevisiae,H. capsulatum, N. crassa, and tobacco leaves. Second, mild alkaline methanolysis is used to destroy the ester-containing lipids in crude or semipurified extracts; however, stronger alkaline conditions should be avoided owing to the lability of unsubstituted inositol attached by a phosphodiester bond. Finally, liquid chromatography on silica gel columns is used for isolating InsPCers; low levels of salt in the eluting solvents are required to chromatograph macroscopic quantities of the very polar InsPCers. A solvent polar enough to dissolve a practical amount of the very polar InsPCers yields insufficient retention. Inclusion of salt increases the retention presumably by providing a charged surface for the negatively charged InsPCers. In contrast, the salt had little influence on the retention of neutral lipids.

Extraction and Purification of Galactosylceramides

Cell culture

Human kidney cells (PT cells) are prepared as described previously (Chatteree et al., Proc. Natl. Acad. Sci. (1983)). Human cadaver kidneys (post mortem<12 hr) are used for the isolation of PT cells. Glucosylceramide was prepared from the spleen of a Gaucher's patient. Digalactosyklceramide is prepared from the kidney of a Fabry's patient. Other GSLs are purchased from Sigma. Normal rat kidney cells were purchased from the American Tissue Culture Collection (Rockville, Md.). Cells (×10⁴) are seeded in 24 well trays and grown for 6 days in medium containing 10% fetal calf serutn (Hyclone, UT).

Lipid Extraction and Fractionation of GSL from Human Kidney

Total lipids are extracted from freeze-dried cultured PT cells or human kidney cortex or both by vigorous homogenization and extraction with hot (50° C.) chloroform:methanol 2:1 (v/v) 10 ml/mg protein. The lipid extracts are filtered on a sintered disc tunnel and the non-lipid residue is extracted further with hot chloroform-methanol 1:2 (v/v) containing 5% H20. Lipid extraction is pursued at 50° C. for 30 mm with constant stirring. The lipid extract is filtered on a sintered glass funnel and the residue is extracted further, first with hot chloroform:methanol:water 1:2, 5%) and then with methanol. The lipid extracts are pooled and dried by flash evaporation. Water-soluble contaminants are removed from the lipid extracts.

Isolation and Purification of GSL

The lower phase lipid fraction obtained following Folch partitioning of human kidney total lipids is fractionated into neutral lipid, GSL and phospholipids by silicic acid chromatography as described previously (Chatterjee et al., 1982). The acetone:methanol fraction containing GSL is subjected to alkaline methanolysis, neutralized and dried under a nitrogen atmosphere. The dried residues are solubilized in chloroform:methanol 2:1 (vlv) and subjected to HPTLC on Silica gel HPTLC plates with chloroform:methanol:water (65:25:4, v/v) as the developing solvent. The individual GSLs are identified with aniline diphenylamine (OPA) reagent or iodine vapours. The chromatogram is calibrated with authentic GSL standards of known structure. The total GSL fraction was precipitated with ether and subjected to mild alkali-catalysed methanolysis, dialyzed against water and separated by silicic acid chromatography. The silicic acid column is equilibrated with chloroform:methanol (19:1) and a sample suspended in the same solvent is applied on the column bed. The column is eluted successively with 12, 14, 16, 20, 30 and 50% methanol in chloroform, and the fractions are dried in a nitrogen atmosphere. The various fractions are analysed for the composition of GSLs. Several of the fractions obtained when the column is eluted with 14% methanol in chloroform contained digalactosylceramide. Such preparations of digalactosylceramide arefurther characterized by high-performance liquid chromatography (HPLC) and are utilized for binding to SEB.

Quantitation of GSL

GSLs are quantified by HPLC, after perbenzoylation. An aliquot of perbenzoylated GSL sample is suspended in hexane and subjected to HPLC on a Spherisorb Si-5 column with detection at 230 nm. The amount of GSL is calculated by using a standard curve for the respective GSLs.

Characterization of Galactosylceramides

Acid hydrolysis of GSLs arecarried out followed by TLC of sugars on aluminium-backed silica gel 60 (without indicator) HPTLC plates with the use of 2-propanol-1% sodium borate (3:1, v/v) as the developing solvent. The sugars are localized by spraying the plate with 10% H₂S0₄ in 50% ethanol and heating at 150° C. for 10 min in an incubator. Anomeric linkage of the purified GSL receptor is determined using b-galactosidase, a-galactosidase and b-glucosidase An aliquot of the purtifed GSL receptor is incubated with or without b-galactosidase, b-galactosidase and b-lucosidase in 0.05 M citrate buffer (pH 5.4) containing taurodeoxycholate for 18 h at 37° C. The reaction is terminated with chloroforn:methanol (2:1 vlv) and the lower chloroform layer is subjected to HPTLC. The plate is developed with aniline DPA reagent

GC-MS of GSL

Suitable aliquots of the putative SEB receptor are subjected to acid-catalysed methanolysis (Essehran et at., 1972). The methyl sugars, methyl fatty acids and methyl sphingosines are purified by solvent extraction as described above. They were dried in a nitrogen atmosphere and derivatized by employing trimethylchlorosilane reagent. The derivatized samples (fatty acid methyl esters) suspended in hexane were injected into a Varian-3400 gas chromatograph (DB-wax capillary column, 30 m; J and W Company, California) that is attached to a mass spectrometer ITD-850, Finnigan Ion Trap detector. Helium is used as a carrier gas. Temperature programming from 160° C. to 250° C. at 1° C./min is employed to separate the various fatty acid methyl esters. The TMSi sugars are separated on a DB-5 column using temperature programming from 160° C. to 250° C. at 1° C./min. Data analysis of TMSi sugars is pursued by the use of a Compaq deskpro-2862 computer. Suitable aliquots of the GSL are subjected to microscale permethylation. The gas chromatography column (DB-5 capillary column.. 0.25 mm×30 m) is calibrated with authentic standards of mixtutes of partially methylated alditol acetates (Biocarb. Chemicals, Sweden). Temperature programming is from 160° C. to 250° C. at 1° C./min and from 250° C. to 350° C. at 2° C./min.

### Example 56

Transfection of Thmidine Kinase Gene into Activated Immunocytes

In order that the immunocytes with deleted or inactivated inhibitory receptors do not undergo unlimited proliferation in vivo it is necessary to provide a method for eliminating these cells after they have performed their tumoricidal function in vivo. To achieve this effect, a retrovirus-mediated transfer of a gene encoding a ‘prodrug’, a reagent that confers sensitivity to cell killing following subsequent administration of a suitable drug. is used. Thymidine kinase (Wigler M et al., Cell, 11: 223 (1977); Colbere-Garapin F et al., Proc. Natl. Acad. Sci, 76: 3755 (1979)) is encoded by the cellular, HSV, or vaccinia virus tk genes and the HSV-tk gene is used as a prodrug gene. The HSV-tk gene is transfected into immunocytes by methods given in Example 1. The HSV-tk confers sensitivity to the drug gancyclovir by phosphorylating it within the cell to form gancyclovir monophosphate which is subsequently converted by cellular kinases to gancyclovir triphosphate. This compound inhibits DNA polymerase and causes cell death. The immunocytes are administered to the host. Unopposed proliferation of immunocytes cells deleted of IRTLAs, IRSAs, IRIDLAs in response to tumor associated lipid-based antigens may lead to immunocyte excess. Therefore, after the immunocytes have performed their tumor killing in vivo, gancyclovir is administered in conventional pharmacologic doses which induces apoptosis of HSV-tk transfected immunocytes.

### Example 57

Methods of Preparation of Anti-Idiotype Antibodies

The methods given below are descibed in Schick, M R et al., Methods in Enzymology 178:36-48 (1989) and Kussie, P H et al., Methods in Enzymology 178: 49-63 (1989). The immunization protocol depends on the species from which the Ab1 was derived and the host animal to be immunized. Typically,mice and rabbits are used to produce anti-Id. To produce Ab2 in a syngeneic animal (e.g., the Ab1 is a mouse MAb and a mouse of the same strain is to be immunized), an Ab1 KLH (keyhole limpet hemocyanin) conjugate as an alum precipitate is used to increase the immunogenicity of the antibody. Four to eight biweekly injections of 50 ug of an Ig: KLH preparation results in maximum Ab2 titers when the mice are immunized intraperitoneally. When using an Ab1 preparation from a different species (e.g., human) to immunize mice, the antibody is not coupled to KLH but instead an alum precipitate of the Ab1 is used.. The time between injections remains the same. Regardless of the Ab1 used, serum is taken 7-14 days following each injection. For Ab2 production in rabbits, the KLH coupling is omitted and the antibody is mixed in Freund's complete adjuvant (CFA; Difco Laboratories, Detroit, Mich.). Rabbits are immunized intramuscularly with between 200 ug and 2 mg of Ab1 per injection. Additional immunizations are in Freund's incomplete adjuvant and are spaced approximately 1 month apart. Serum is taken 14-30 days following each immunization. Rabbits have received up to a total of nine immunizations before an anti-Id of the desired specificity and titer is obtained.

The disadvantage of a heterologous immunization protocol where the Ab1 and Ab2 are obtained from different species is that antibodies are produced that recognize isotypic and allotypic specificities, along with anti-Id. In the instance where a monoclonal Ab2 is desired, the initial screening process can select the anti-Id versus the anti-isotype- and/or -allotype-secreting clones of hybridoma cells. An anti-isotype response recognizes an irrelevant Ig preparation from the same species and the Ab 1, whereas the anti-Id recognizes only the Ab 1 and not the irrelevant Ig preparation. An antiallotype response recognizes both a preimmune Ig preparation obtained from the Ab1 source prior to immunization with the antigen and the Ab1 preparation, while the anti-Id will recognize only the Ab1 preparation but not the preimmune 1 g. Based on the distinction between an antiallotype versus anti-Id in the initial screening and characterization process, it is advantageous to obtain antibodies from the Ab1 source, preferably the donor, prior to immunization.

If one is not generating a monoclonal anti-Id but rather a polyclonal anti-Id and the Ab1 is from a different species, then the antiserum must be adsorbed to remove anti-isotypic and antiallotypic specificities and render the antiserum anti-Id specific. Each immunoadsorbent is prepared by covalently coupling nonimmune Ig at a concentration of 3-4 mg/ml of antibody per 1 ml of CNBr-activated Sepharose 4B or Affi-Gel 10. Antisera containing anti-Id as well as antibodies to iso- and allotypic determinants is repeatedly adsorbed on the imrnunosorbents until all detectable reactivity against nonidiotypic determinants is removed.

KLH Coupling and Alum Precipitation

Adsorption of immunoglobulins to alum particles, resulting in aggregation, increases the preparations and is tolerated very well in mice even after multiple injections. Purified antibodies to be coupled to a carrier protein, such as KLH, are diluted to 5 mg/ml in borate-buffered saline (BBS), pH 8.2, and cooled to 4°. The antibodies are then mixed with a 10,000: 1 molar ratio of 1-ethyl-3-(3-dimethylaminopropyl)carbodiimide (EDAC; Sigma Chemical Co., St. Louis, Mo.) to 1 g. The separation of Ig:Ig conjugates from Ig:KLH does not enhance the ability of this preparation to induce an Ab2 response.) The mixture is allowed to stir at 4° for 30 sec. KLH is added to the Ig-EDAC mixture and stirred for 2 hr at 25° and then overnight at 4° A molar ratio of 50:1 Ig to KLH is used. The Ig:KLH conjugate is dialyzed against BBS overnight at 4° and then adsorbed to alumina. Briefly, 10% aluminum potassium sulfate (w/v) is dissolved in 5 mM PBS (phosphate-buffered saline), pH 6.2, resulting in a 5.7 mg/ml alumina/ml solution. Eight milligrams of alumina is added slowly to 1 mg of protein. The pH is adjusted to 6.8-7.3 with 1 N NaOH. The mixture is allowed to stir for 2 hr at 25° and is then centrifuged at 1300 g for 10 mm. The supernatant is examined for unadsorbed protein at 280 nm. The pellet is washed three times in 0.85% NaCl and then resuspended to 500 ug/ml in BBS and stored at 4° with 0.01% thimerosal (Sigma).

Ab2 Detection and Characterization

Other types of determinants expressed on antibodies which have immunogenic capabilities are isotypic and allotypic as well as idiotypic specificities. When immunizing disparate species, it is necessary to take into account the anti-isotype and antiallotype antibodies which will be produced These antibodies are removed when present, by exhaustively adsorbing anti-Id-containing sera with normal immunoglobulin covalently coupled to Sepharose. These steps are taken only when using larger animal species to produce Ab2. When immunizing nice with Ab1 from another species, we do not adsorb the sera; instead, the mice are used to produce monoclonal anti-Id. The reactivity against nonspecific Ig is determined as well as specificity for the AbI. If desired, the mouse sera can be adsorbed to examine the anti-Id response prior to fusion.

Ab2 can be detected in a direct binding sandwich ELISA. Wells of an ELISA plate are coated with 100-500 ng of Ab1. Serum or hybridoma supernatant is then allowed to react with the adsorbed Ab1 for 1 hr at 37°. Because antibody molecules are bimodal and flexible, it is possible to add labeled Ab1 as a detecting antibody. The Ab2 can then bind to the solid-phase Ab1 with one arm while the other arm is available for binding labeled Ab1. This method is the least sensitive for detecting an anti-Id response. Alternatively, if the Ab1 and Ab2 are from different species, an anti-isotype reagent can also be used to detect Ab2 binding. As previously mentioned, care should be taken to distinguish anti-isotype and antiallotype activity from anti-Id activity.

If the Ab1 preparation is a murine 1 gm, we can utilize an indirect ELISA whereby anti-Id binding to the 1 gM Ab1 present on the solid phase is detected by a goat anti-mouse IgG Fc-HRP (horseradish peroxidase) or biotinylated reagent (Kirkegaard and Perry Laboratories, Gaithersburg, Md.). These assays demonstrate more sensitivity in detecting an anti-Id response compared with the direct binding sandwich ELISA. For murine lgG Ab1 preparations, papain or pepsin digestions can be performed to produce Fab and/or Fab fragments that are devoid of the Fc region. The Fab- or Fab₂′-derived Ab1 can be adsorbed to the solid phase, and anti-Id binding can be detected similarly by utilizing a goat anti-mouse IgG Fc second antibody reagent.

When the Ab1 and Ab2 are from different species and the appropriate adsorptions have been performed on polyclonal Ab2-containing antisera to render it anti-Id specific, assays utilizing a second antibody reagent, such as a goat anti-rabbit IgG, are useful for detecting a rabbit anti-Id binding to the Ab1.

A polyclonal Ab2 may contain multiple Ab2 specificities. It is also possible that the quantity as well as the specificity of the Ab2 populations produced may change with multiple immunizations. Thus, samples are tested especially prior to pooling sera from different samplings or indi-vidual animals. Only methods for determining Ab2□, Ab2□. and Ab2□ are discussed here.

Ab2□ and Ab2□ share the ability to bind to the antigen combining site of the Ab1. This feature can be detected using an inhibition assay. If purified antigen recognized by the Ab1 is available, it can be used as a solid-phase coat in an ELISA. For tumor antigens, 1-200 ng of purified antigen (Ag) is added to each well and allowed to bind for 18 hr at 4° in 10 mM carbonate buffer, pH 9.6. Other antigens will, of course, differ in their binding requirements, and optimum conditions for each will have to be determined. Sera or hybridoma supernatant containing Ab2 must be used in an attempt to inhibit the binding of Ab1 to the Ag coat. Inhibition of Ab1-Ag binding can be detected by using labeled Ab1 preparations such as biotinylated Ab1 or HRP-conjugated Ab1. By definition on Ab2□ will not inhibit binding in this ELISA whereas an Ab2□ and an Ab2□ should. An alternative method is to coat with the Ab1 first so that it inhibits the binding of an Ag preparation by the Ab2 in a competitive inhibition ELISA. Antigen binding can be detected by Ab1 or other antisera which recognize the Ag.

Distinguishing between Ab2□ and Ab2□, both of which block Ab1-Ag binding, is most easily accomplished using antisera to the Ag obtained from several different species. Depending on the titer of the antisera used, it may be necessary to affinity purify the xenogeneic Ab1 preparations. An Ab2□ should recognize the Id of antibodies from these other species if it represents the internal image of the antigen and exhibits serological minicry. Ab2□, however, should not bind to these Ab1 antibodies produced in other species. It must be kept in mind that an Ab2□ or an Ab2□ which has a higher affinity for the Ab1 than does the Ag for the Ab1 may appear as Ab2□ preparations.

Monoclonal Anti-Idiotypic Antibodies: Immunization Procedures

For the production of monoclonal antibodies to small ligands the following parameters are considered (1) conjugation of the ligand to a carrier molecule necessary to create the immunogen, (2) different strains of mice or hamsters as the immunized hosts, (3) use of adjuvants with the immunogen, and (4) route of immunogen presentation. For almost all protocols, small ligands are conjugated to whelk hemocyanin (Busycon canaliculatum, Marine Biological Labs, Woods Hole, Mass.) or bovine serum albumin. Careful consideration of the position for ligand conjugation must be made so as not to interfere with the pharmacophore. In addition, the type of chemical linkage or linker arm must also be considered.

Although BALB/c mice are almost always used for hybridomas, one should consider the use of F₁ hybrids which may offer an expanded major histocompatibility complex (MHC)-directed and immunoglobulin gene-controlled immune repertoire. F₁ hybrids produced from BALB/c with SJL/J, CBA/J, and NZB matings often provide this increased immune responsiveness. Hybridomas derived from F₁ hybrids can be fed in vitro using spleen cells from F₁ donor mice or conditioned media; growth of such hybridomas as ascites tumors requires F₁ pristane-primed mice. In some instances inbred strains of mice may not offer the necessary immune repertoire for a particular ligand, and we have turned to the use of interspecies hybridomas using Armenian hamsters (Ardago Farms, Brenham, Tex.). For the most part, the immunization protocols are identical to those used for mice. Such interspecies hybridomas cannot be easily grown as ascites tumors.

The immunization procedures are carried out as follows: Animals are immunized with 50-100 ug of ligand-conjugated hemocyanin or albumin emulsified in Freund's complete adjuvant; each leg is injected with 25 ul for a total volume of 100 ul. The Ribi Adjuvant system (Ribi Immunochem Research, Hamilton, Mont.), which utilizes a mixture of a monophosphoryl lipid A and trehalose dimycolate as a substitute for Freund's complete adjuvant is also useful. In most instances some killed Bordetella pertussis organisms are added (10⁸) as an additional adjuvant for B cells. Booster immunizations of 50-100 ug immunogen (2-4 subcutaneous or intramuscular sites) are given 4-5 weeks later, and the first test bleeds are taken 7-10 days later. Seropositive animals are allowed to ‘rest” for 20-40 days prior to use for hybridoma production. The preftision antigen boosters, which are given 4 days prior to the harvest of spleens for hybridomas, have traditionally been given intraperitoneally or intravenously; we have noted that much of the antigen does not reach the desired tissue site, namely, the spleen, and therefore prefer the intrasplenic injection procedure.

The procedure for intrasplenic injections requires only a few minutes. Animals are anesthetized under a “nose-cone” with Metafane brand of methoxyfiurane (Pitman Moore, Washington Crossing, N.J.), and a small incision on the left-hand side of the peritoneum is made using sterile scissors. The spleen is gently lifted out using small forceps, and the soluble immunogen (25-50 ug) in 100 ul volume of saline is injected using a 1-mi tuberculin syringe and a 26-gauge needle; the spleen is returned to the peritoneal vault, and two surgical clips are used to close the wound. The animals recover within a few minutes with no apparent ill affects. This procedure provides a direct bolus of antigen to the splenic immunocytes and, in most instances, produces more hybridomas than other booster procedures. Immunogen is also adsorbed on nitrocellulose, and subsequent injection of homogenized fragments of this material acts as a carrier for the intrasplenic retention of immunogen.

Affinity Purification of the SEB C-Terminal Region-Specific Abs

The method for obtaining purified antibodies to the dominant epitope in SEB reactive with circulating immunoglobulings is given below (Nishi, J I et at, J. Immunol. 158:247-254 (1997)). However, the same principles would apply to the dominant immunizing epitopes of all the other superantigens. To obtain Abs recognizing this assumed epitope region, SEB C-terminal region-specific lgG from iv. IgG is purified by means of affinity chromatography on CNBr-activated Sepharose 4B coupled with the recombinant fusion protein C. The bound IgG is eluted and dialyzed, and the reactivities of this IgG with the recombinant fusion proteins examined by immunoblotting. Purified IgG recognizes only fusion protein C. This finding showed that this purified IgG mainly recognizes the assumed dominant epitope region. The SEB is similar to the superantigen SECI and SPEA, and not to TSST-1, SEA, and SEE. Since the SEB C-terminal region, including that of the assumed epitope region, has homology with those of SECI and SPEA, the reactivity of each superantigen with the SEB C-terminal region-specific Ab by means of an ELISA is examined. The Ab recognized SEB and cross-reacted with SECl. It rarely reacted with SPEA, and never with TSST-1. SEA, and SEE.

Affinity adsorption of specific Abs against the full-length SEB and the mutant A 225-234 protein is carried out as follows: Pooled lgG (10 mg) is adsorbed by mixing with the CNBr-activated Sepharose 4B coupled with the full-length SEB protein (5 mg) or the mutant A 225-234 protein (5 mg) for 1 h at room temperature. Each adsorbed lgG is removed by centrifugation and used for lymphocyte proliferation assay,

Additional Documents Incorporated by Reference

This application incorporates by reference the following patents and currently pending patent applications that disclose inventions of the present inventor alone or with co-inventors.

1. Patent application WO91/US342, “Tumor Killing Effects of Enterotoxins and Related Compounds” filed Jan. 17, 1991, and published as WO 91/10680 on Jul. 25, 1991.

2. U.S. Ser. No. 07/891,718 “Tumor Killing Effects of Enterotoxins and Related Compounds,” filed Jun. 1, 1992.

3. U.S. Pat. No. 5,728,388, “Method of Cancer Treatment,” issued Mar. 17, 1998.

4. U.S. Ser. No. 08/491,746, “Method of Cancer Treatment,” filed Jun. 19, 1995.

5. U.S. Ser. No. 08/898,903 “Method of Cancer Treatment,” filed Jul. 23, 1997.

6. U.S. Ser. No. 08/896,933 “Tumor Killing Effects of Enterotoxins and Related Compounds,” filed Jul. 18, 1997.

7. U.S. Ser. No. 60/085,506, “Compositions and Methods for Treatment of Cancer,” filed May 5, 1998.

8. U.S. Ser. No. 60/094,952 “Compositions and Methods for Treatment of Cancer” filed Jul. 31, 1998.

9. U.S. Ser. No. 60/033,172 “Superantigen-Based Methods and Compositions for Treatment of Cancer,” filed Dec. 17, 1996.

10. U.S. Ser. No. 60/044,074 “Superantigen-Based Methods and Compositions for Treatment of Cancer,” filed Apr. 17, 1997.

11. U.S. Ser. No. 09/061,334 “Tumor Cells with Increased Immunogenicity and Uses Thereof,” filed Apr. 17, 1998.

11. U.S. Ser. No. 09/311,581 “Compositions and Methods for Treating Neoplastic Disease,” filed May 14, 1999.

12. U.S. Ser. No. 09/650.884 “Compostitions and Methods for Treating Neoplastic Disease,” filed Aug. 30, 2000.

Moreover, all references cited herein are incorporated by reference, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

What is claimed is:

1. A receptor in a mammalian cell useful in the treatment of cancer which inhibits cellular activation by receptors specific for lipid-based tumor associatiated antigens.

2 The receptor of claim 1 wherein the lipid antigen is a bacterial, fungal, protozoal or mycobacterial antigen.

3. The inhibitory receptor of claims 1 and 2 wherein said inhibitory receptor contains an inhibitory receptor tyrosine-based inhibitory motifs (ITIMs).

4. The inhibitory receptor of claim 1, 2 wherein said receptor is specific for lipid-based tumor associated antigen and/or self MHC or CD1 molecules.

5. A receptor in a mammalian cell wherein said receptor inhibits cellular activation by receptors specific for lipid-based infectious disease associated antigens derived from bacteria, fungi, mycobacterium, parasite, virus, eukaryote or prokaryote antigens in the context of MHC or CD1.

6. A mammalian cell useful in the treatment of cancer wherein the inhibitory receptor for lipid-based tumor associated antigens is deleted or functionally deactivated.

7. A mammalian cell useful in the treatment of cancer wherein inhibitory receptor tyrosine-based inhibitory motifs of the inhibitory receptor for lipid-based tumor associated antigens are deleted or functionally deactivated.

8. A mammalian cell useful in the treatment of cancer wherein the the inhibitory receptor for superantigens associated with self antigens are functionally deleted.

9. A mammalian cell useful in the treatment of cancer wherein the inhibitory receptor for tumor associated lipid antigens and superantigens are deleted or functionally deactivated.

10. The lipid-based tumor associated antigens of claims 1, 2, 4, 6, 9, wherein said lipid-based tumor associated antigen is selected from the group consisting of glycolipids, proteolipids, glycosphingolipids, sphingolipids, gangliosides, phytoglycolipids.

11. The lipid antigens derived from bacteria, mycobacteria, fungi and protozoa marine invertebrates of claim 2 wherein said lipid antigens are selected from the group consisting of glycosylceramides, glyco lipids, proteolipids, glyco sphingo lipids, gangliosides and sphingolipids with inositolphosphate-containing head groups, phytoglycolipids, mycoglycolipids, lipoarabinan and mycolic acid.

12 The sphingolipid antigens of claim 11 wherein the sphingolipid contains inositolphosphate-containing head groups with the general structure of ceramide-P-myoinositol-X with X referring to polar substituents consisting of ceramide-p-inositol-mannose, inositol-1-P-(6)mannose(a1,2inositol-1P-(1)ceramide, (inositol-P)2-ceramide, inositol-P-inositol-P-ceramide, inositol-P-inositol-P-ceramide.

13. The mammalian cell of claim 6-9 wherein said cell is an immunocyte selected from a group consisting of T cell, NK cell, NKT cells

14. A mammalian cell of claim 7 wherein the superantigen is selected from a group consisting of a staphylococcal enterotoxin, a streptococcal pyrogenic exotoxin, mycoplasma arthitides, rabies antigen, clostridial product.

15. A mammalian cell useful in the treatment of cancer wherein the inhibitory receptor for glycan-based tumor associated antigens is deleted or functionally deactivated.

16. The glycan antigens of claim 15 wherein said glycan antigen is selected from the group consisting of peptidoglycans or glycan phosphotidylinositol (GPI) structures.

17. A mammalian cell useful in the treatment of cancer wherein the the inhibitory receptor for superantigen-associated self antigens are functionally deleted or inactivated.

18. The self antigens of claims 17 wherein said self antigens consist of a MHC or CD1 molecule.

19. A mammalian cell useful in the treatment of cancer wherein the inhibitory receptors and/or immune receptor tyrosine based inhibitory motifs which inhibits cellular activation by receptors specific for lipid-based tumor associated antigens and superantigens are deleted or functionally deactivated.

20. The superantigen of claims 17 wherein said superantigen is selected from a group consisting of the staphylococcal enterotoxins SEA, SEB, SEC, SEC1, SEC2, SEC3, SED, SEE, TSST-1 or streptococcal pyrogenic exotoxins, mycoplasma arthritides, rabies virus, mammary tumor virus, clostridial antigen.

21. A mammalian cell in which the inhibitory receptor for lipid-based infectious disease associated antigens and/or immune receptor tyrosine based inhibitory motifs which inhibits cellular activation by receptors specific for lipid-based infectious disease associated antigens derived from bacteria, fungi, mycobacteria, parasite, virus, eukaryote or prokaryote antigens are deleted or functionally deactivated.

22. The mammalian cell of claims 13, 14, 15, 17, 18, 21 wherein said cell is an immunocyte selected from a group consisting of T cells, NK cells, NKT cells

23. The lipid antigens of claims 21 wherein said lipid-based infectious disease associated antigen or fatty acid is mycolic acid or lipoarabinan,

24 A method of treating cancer in a mammal, said method comprising inactivating or deleting inhibitory receptors or immune receptor tyrosine based inhibitory motifs in immunocytes which inhibit activating receptors specific for lipid-based tumor associated lipid antigens or superantigens.

25. A method of inactivation or deletion of receptors or ITIMs in immunocytes which inhibit cell activating receptors specific for lipid-based tumor associated antigens and superantigens comprising inactivation or deletion of nucleic acids encoding ITIMs.

26. A method for producing a tumoricidal immunocyte population in vivo said method comprising allowing a tumor associated lipid antigen and superantigen to contact immunocyte activation receptors specific for tumor associated lipid antigens and superantigens in which inhibitory receptors or ITIMs which inhibit said cell activation by receptors specific for lipid-based tumor associated antigens are inactivated or deleted.

27. A method for producing a tumoricical immunocyte population ex vivo, said method comprising:
a) allowing a lipid-based tumor associated antigen and superantigen to contact immunocyte activation receptors specific for lipid-based tumor associated antigens and superantigens in which inhibitory receptors or ITIMs which inhibit said cell activating receptors for lipid-based tumor associated antigens are deleted or inactivated.
b) administering said tumoricidally activated immunocytes to the host.

28. A method of producing a immunocyte population effective against infectious disease in a mammal in vivo said method comprising:
a) allowing a lipid-based infectious disaease associated antigen and superantigen to contact immunocyte activation receptors specific for and superantigens in which inhibitory receptors or ITIMs which inhibit said cell activation receptors specific for lipid-based infectious disaease associated antigen and superantigens are inactivated or deleted.

29. A method for producing an immunocyte population effective against infectious disease in a mammal ex vivo, said method comprising:
a) allowing a lipid-based infectious disease associated antigen and superantigen to contact immunocyte activation receptors specific for lipid-based infectious disease associated antigens and superantigens in which inhibitory receptors or inhibitory receptors with tyrosine-based inhibitory motifs which inhibit said cell activating receptors for lipid-based infectious disease associated antigens are deleted or inactivated.
b) administering said immunocyte population effective against infectious disease to the host.

30. The immunocytes of claims 26-29 wherein the said immunocytes comprise a group consisting of a T cell, NK cell or NKT cell

31. The immunocytes of claim 27, 29 wherein the said immunocytes are expanded in cytokines ex vivo prior to said administration

32. The method of claims 24-29 wherein said superantigen comprises a staphylococcal enterotoxin, a streptococcal pyrogenic exotoxin, mycoplasma arthritites, rabies virus, clostridial antigen, heat shock protein.

33. The staphylococcal enterotoxin of claim 32, wherein said enterotoxin is selected from the group consisting of SEA, SEB, SEC1, SEC2, SED, SEE, SEF, TSST-1, SPEA, SPEB, SPEC, Streptococcal pyogenic exotoxin.

34. The superantigen of any of the claims wherein said superantigen is expressed by a tumor cell or accessory cell which has been transfected with a nucleic acid encoding a superantigen.

35. The superantigen of claims 34 wherein said superantigen is expressed on the surface of a cell.

36. The cell of claim 35 wherein said cell is a tumor cell or an accessory cell.

37. The superantigen transfected tumor cell or accessory cell of claims 34-36 comprising transfecting said transfected cell with additional nucleic acids selected from a group comprising an adhesion molecule, an MHC molecule, a costimulatory molecule or a plurality thereof wherein said transfected cell expresses said encoded molecule(s) from said nucleic acid.

38. The transfected tumor cell or accessory cell of claims 34-37 wherein said transfected cell is transfected in vivo.

39. The transfected tumor cell or accessory cell of claims 34-37 wherein said transfected cell is transfected ex vivo.

40. A mammalian cell wherein inhibitory receptors or their ITIMs and Fas ligand receptors are deleted or functionally inactivated

41. The mammalian cell of claim 30, 31 wherein said cell is an immunocyte selected from a group consisting of T cell, NK cell, NKT cells

42. A method of treating cancer by wherein lipid-based tumor associated antigen or superantigen agonist motifs selectively contact immunocyte activating receptors and not immunocyte inhibitory receptors in vivo thereby producing an immunocyte population which is effective in the treatment of cancer.

43. A method of treating cancer by wherein lipid-based tumor associated antigen or superantigen agonist motifs selectively contact immunocyte activating receptors and not immunocyte inhibitory receptors ex vivo thereby producing an immunocyte population which is administered to the host and is effective in the treatment of cancer.

44. A method of treating infectious disease wherein lipid based infectious disease associated antigen agonist motifs selectively contact immunocyte activating receptors and not immunocyte inhibitory receptors in vivo thereby producing an immunocyte population effective in the treatment of infectious disease.

45. A method of treating infectious disease wherein lipid-based infectious disease associated antigens or superantigen agonist motifs selectively contact immunocyte activating receptors and not immunocyte inhibitory receptors ex vivo thereby producing an immunocyte population which is administered to the host and is effective in the treatment of infectious disease.

46. A method of treating infectious disease wherein lipid-based tumor associated antigens, lipid-based infectious disease associated antigens or superantigen antagonist motifs are deleted or blocked from contact with immunocyte inhibitory receptors thereby allowing agonist motifs to stimulate immunocyte activating receptors to produce an immunocyte population which is effective in the treatment of cancer or infectious disease.

47. A method of treating cancer and infectious disease according to claims wherein the immunocytes are transfected with HSV thymidine kinase gene which induces immunocyte death in vivo in response to exogenous administration of gancyclivir.

48. An mammalian antigen presenting cell wherein MHC class I molecules molecules of said cell are deleted or inactivated rendering said cell capable of presenting tumor associated lipid antigens and superantigens to immunocytes which are then capable of inducing a tumoricidal response,

49. An mammalian antigen presenting cell wherein MHC class I molecules molecules of said cell are deleted or inactivated rendering said cell capable of presenting infectious disease associated lipid antigens and superantigens to immunocytes which induce an effective response against infectious disease.

50. A mammalian cell comprising a fusion of a tumor cell with a mammalian cell whereby said fusion cell expresses glycosylceramides and tumor antigens.

51. A mammalian cell comprising a fusion of a tumor cell with a mammalian or invertebrate cell whereby said fusion cell expresses tumor antigens and phytosphingolipids.

52. The fusion cells of claims 50, 51 wherein the said fusion cells are transfected with superantigen genes whereby said fusion cell expresses a superantigen.

53. A pharmacuetical composition useful in treatment of cancer comprising a lipid-based tumor associated antigen conjugated to a superantigen.

54. The composition of claim 53 wherein the lipid-based tumor associated antigen is selected from a group consisting of a glycolipid, proteolipid, glycosphingolipid, ganglioside.

55. A pharmaceutical preparation useful in the treatment of infectious disease comprising a lipid-based infectious disease associated antigen conjugated to a superantigen

56. The composition of claim 55 wherein the infectious disease associated lipid antigen is selected from a group consisting of a glycolipid, proteolipid, glycosphingolipid, ganglioside , phytosphingolipid, mycosphingolipid, lioarabinan or mycolic acid

57. The composition of claim 56 wherein the sphingolipid contains inositolphosphate-containing head groups with the general structure of ceramide-P-myoinositol-X with X referring to polar substituents consisting of ceramide-p-inositol-mannose, inositol-1-P-(6)mannose(a1,2inositol-1P-(1)ceramide, (inositol-P)2-ceramide, inositol-P-inositol-P-ceramide, inositol-P-inositol-P-ceramide.

58. A pharmacuetical composition useful in the treatment of cancer comprising a tumor associated glycan antigen conjugated to a superantigen.

59. The composition of claim 58 wherein the glycan is selected from a group consisting of a peptidoglycan or glycan-phosphotidyinositol (GPI) structures.

60. The compositions of claims 53-59 wherein the conjugates are bound to an MHC or CD1 receptor.
